SEQUENCE LISTING (347 sequences)

SEQ ID NO: 1 - protein, 8 aa, synthetic construct
GYSITSGY

SEQ ID NO: 2 - protein, 5 aa, synthetic construct
SYDGY

SEQ ID NO: 3 - protein, 11 aa, synthetic construct
YYDYEDWYFGV

SEQ ID NO: 4 - protein, 15 aa, synthetic construct
RASESVSIIGTNSIH

SEQ ID NO: 5 - protein, 7 aa, synthetic construct
HASNLET

SEQ ID NO: 6 - protein, 9 aa, synthetic construct
LQSRKIPYT

SEQ ID NO: 7 - protein, 6 aa, synthetic construct
SGYYWN

SEQ ID NO: 8 - protein, 16 aa, synthetic construct
YISYDGYNNYNPSLKN

SEQ ID NO: 9 - protein, 120 aa, synthetic construct
DVQLQESGPGLVKPSQSLSLTCSVTGYSITSGYYWNWIRQFPGNKLEWMGYISYDGYNNYNPSLKNRISITRDTSKNQFFLKLNSVTTEDTATYYCANYYDYEDWYFGVWGTGTTVTVSS

SEQ ID NO: 10 - protein, 111 aa, synthetic construct
DIVLTQSPASLAMSLGKRATISCRASESVSIIGTNSIHWYQQKPGQPPKLLIYHASNLETGVPARFSGSGSRTDFTLTIDPVEEDDVAIYYCLQSRKIPYTFGGGTKLEIK

SEQ ID NO: 11 - protein, 7 aa, synthetic construct
GYTFTDY

SEQ ID NO: 12 - protein, 6 aa, synthetic construct
YPLRGS

SEQ ID NO: 13 - protein, 11 aa, synthetic construct
HGAYYSNAFDY

SEQ ID NO: 14 - protein, 15 aa, synthetic construct
RASESVDNDGIRFMH

SEQ ID NO: 15 - protein, 7 aa, synthetic construct
RASNLES

SEQ ID NO: 16 - protein, 9 aa, synthetic construct
QQSNKDPYT

SEQ ID NO: 17 - protein, 5 aa, synthetic construct
DYTIH

SEQ ID NO: 18 - protein, 17 aa, synthetic construct
WIYPLRGSINYNEKFKD

SEQ ID NO: 19 - protein, 120 aa, synthetic construct
QVQLQQSGAELVKPGASVRLSCEASGYTFTDYTIHWVKQRSGQGLEWIGWIYPLRGSINYNEKFKDKATLTADKSSSTVYLELGRLTSKDSAVYFCARHGAYYSNAFDYWGQGTTLTVSS

SEQ ID NO: 20 - protein, 111 aa, synthetic construct
NIVMTQSPASLAVSLGQRATISCRASESVDNDGIRFMHWYQQKPGQPPKLLIYRASNLESGIPARFSGSGSRTDFTLTINPVETDDVATYYCQQSNKDPYTFGGGTKLELK

SEQ ID NO: 21 - protein, 7 aa, synthetic construct
GYTFTSY

SEQ ID NO: 22 - protein, 6 aa, synthetic construct
YIGNGY

SEQ ID NO: 23 - protein, 7 aa, synthetic construct
YYPWFTY

SEQ ID NO: 24 - protein, 11 aa, synthetic construct
RASENIYSYLA

SEQ ID NO: 25 - protein, 7 aa, synthetic construct
NAKTLAE

SEQ ID NO: 26 - protein, 9 aa, synthetic construct
QHHYDTPFT

SEQ ID NO: 27 - protein, 5 aa, synthetic construct
SYGIN

SEQ ID NO: 28 - protein, 17 aa, synthetic construct
YIYIGNGYAEYNERFKG

SEQ ID NO: 29 - protein, 116 aa, synthetic construct
EVQLQQSGAELVRPGSSVKMSCKTSGYTFTSYGINWVKQRPGQGLEWIGYIYIGNGYAEYNERFKGKATLTSDTSSSTAYMQLSSLTSEDSAIYFCALYYPWFTYWGQGTLVTVSA

SEQ ID NO: 30 - protein, 107 aa, synthetic construct
DIQMTQSPASLSASVGDSVTITCRASENIYSYLAWYQQKQGKSPQLLVYNAKTLAEGVPSRFSGSGSGTQFSLKINSLQPEDFGNYYCQHHYDTPFTFGGGTKLEIK

SEQ ID NO: 31 - skipped

SEQ ID NO: 32 - protein, 6 aa, synthetic construct
YPRDSS

SEQ ID NO: 33 - protein, 12 aa, synthetic construct
EGYDYDKRGFDY

SEQ ID NO: 34 - protein, 15 aa, synthetic construct
KASQSVSFAGTNLMH

SEQ ID NO: 35 - protein, 7 aa, synthetic construct
RASNLEP

SEQ ID NO: 36 - protein, 9 aa, synthetic construct
QQSREYPWT

SEQ ID NO: 37 - protein, 5 aa, synthetic construct
SYDVF

SEQ ID NO: 38 - protein, 17 aa, synthetic construct
WIYPRDSSTKYNEKFKG

SEQ ID NO: 39 - protein, 121 aa, synthetic construct
QVQLHQSGPELVKPGASVKLSCKTSGYTFTSYDVFWVKQRPGQGLEWIGWIYPRDSSTKYNEKFKGKATLTVDTSSSTAYMELHSLTSEDSAVYFCAKEGYDYDKRGFDYWGQGTTLTVSS

SEQ ID NO: 40 - protein, 111 aa, synthetic construct
DIVLTQSPASLAVSLGQRAIISCKASQSVSFAGTNLMHWYQQRPGQQPKLLIYRASNLEPGVPTRFSGSGSRTDFTLNIHPVEEDDAATYYCQQSREYPWTFGGGTKLEIK

SEQ ID NO: 41 - skipped

SEQ ID NO: 42 - protein, 6 aa, synthetic construct
DPETGG

SEQ ID NO: 43 - protein, 6 aa, synthetic construct
WNDGDY

SEQ ID NO: 44 - protein, 16 aa, synthetic construct
KSSQSLLYSNGKTYLN

SEQ ID NO: 45 - protein, 7 aa, synthetic construct
QVSKLDP

SEQ ID NO: 46 - protein, 9 aa, synthetic construct
LQGTYYPYT

SEQ ID NO: 47 - protein, 5 aa, synthetic construct
DYEMH

SEQ ID NO: 48 - protein, 17 aa, synthetic construct
AIDPETGGTAYNQRFKG

SEQ ID NO: 49 - protein, 115 aa, synthetic construct
QVQLQQSGAELVRPGASVTLSCKASGYTFTDYEMHWVKQTPVHGLEWIGAIDPETGGTAYNQRFKGKAILTTDKSSITAYMELRSLTSEDSAVYYCTRWNDGDYWGQGTTLTVSS

SEQ ID NO: 50 - protein, 112 aa, synthetic construct
DVVMTQTPLSLSVTIGQPASISCKSSQSLLYSNGKTYLNWFQQRPGQSPKRLMYQVSKLDPGIPDRFSGSGSETDFTLKISRVEAEDLGLYYCLQGTYYPYTFGGGTKLEIK

SEQ ID NO: 51 - protein, 7 aa, synthetic construct
GYSFTGY

SEQ ID NO: 52 - protein, 6 aa, synthetic construct
NPYNGD

SEQ ID NO: 53 - protein, 11 aa, synthetic construct
EGDGYYWYFDV

SEQ ID NO: 54 - protein, 15 aa, synthetic construct
RASESVDNYGISFMN

SEQ ID NO: 55 - protein, 7 aa, synthetic construct
AASNQGS

SEQ ID NO: 56 - protein, 9 aa, synthetic construct
QQSKEVPRT

SEQ ID NO: 57 - protein, 5 aa, synthetic construct
GYFMN

SEQ ID NO: 58 - protein, 17 aa, synthetic construct
RINPYNGDTFYNQKFKG

SEQ ID NO: 59 - protein, 120 aa, synthetic construct
EVQLQQSGPELVKPGASVKMSCKASGYSFTGYFMNWVKQSHGKSLEWIGRINPYNGDTFYNQKFKGKATLTVDKSSSTAHMELRSLTSEDSALYYCASEGDGYYWYFDVWGAGTTVTVSS

SEQ ID NO: 60 - protein, 111 aa, synthetic construct
DIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPRTFGGGTKLEIK

SEQ ID NO: 61 - protein, 7 aa, synthetic construct
GYTFTDH

SEQ ID NO: 62 - protein, 6 aa, synthetic construct
DPDTGD

SEQ ID NO: 63 - protein, 6 aa, synthetic construct
WTGGDY

SEQ ID NO: 64 - protein, 5 aa, synthetic construct
DHEMH

SEQ ID NO: 65 - protein, 17 aa, synthetic construct
VIDPDTGDTTYNQKFKG

SEQ ID NO: 66 - protein, 115 aa, synthetic construct
QVQLQQSGAELVRPGASVKLSCKASGYTFTDHEMHWVRQTPVHGLEWIGVIDPDTGDTTYNQKFKGKATLTADKSSSTAYMDLRSLTSEDSAVFYCTRWTGGDYWGHGTTLTVSS

SEQ ID NO: 67 - protein, 16 aa, synthetic construct
KSSQSLLYSDGKTYLN

SEQ ID NO: 68-69 - skipped (2 entries)

SEQ ID NO: 70 - protein, 112 aa, synthetic construct
DAVMTQTPLSLSVTIGQPASISCKSSQSLLYSDGKTYLNWFQQRPGQSPKRLMYQVSKLDPGIPDRFSGSGSETDFTLKISRVEAEDLGVYYCLQGTYYPYTFGSGTKLEIK

SEQ ID NO: 71 - DNA, 360 bp, synthetic construct
  source 1..360: mol_type = other DNA
gatgtacagcttcaggagtcaggacctggcctcgtgaaaccttctcagtctctgtctctcacctgctctgtcactggctactccatcaccagtggttattactggaactggatccggcagtttccaggaaacaaactggaatggatgggctacataagctacgatggttacaataactacaacccatctctcaaaaatcgaatctccatcactcgtgacacatctaagaaccagtttttcctgaagttgaattctgtgactactgaggacacagccacatattactgtgcaaactactatgattacgaagactggtacttcggtgtctggggcacagggaccacggtcaccgtctcctca

SEQ ID NO: 72 - DNA, 333 bp, synthetic construct
  source 1..333: mol_type = other DNA
gacattgtgctgacccaatctccagcttctttggctatgtctctagggaagagggccaccatctcctgcagagccagcgaaagtgtcagtattattggtactaattcaatacactggtaccaacagaaaccaggacagccacccaaactcctcatctatcatgcatccaacctagaaactggagtccctgccaggttcagtggcagtgggtctagaacagacttcaccctcaccattgatcctgtggaggaagatgatgttgcaatctattactgtctgcaaagtaggaagattccgtacacgttcggaggggggaccaagctggaaataaaa

SEQ ID NO: 73 - DNA, 360 bp, synthetic construct
  source 1..360: mol_type = other DNA
caggtccagctgcagcagtctggagctgagctggtgaaacccggggcatcagtgaggctgtcctgcgaggcttctggctacaccttcacggactatactatacactgggtaaagcagaggtctggacagggtcttgagtggattggatggatttaccctctaagaggtagtataaactacaatgagaaattcaaggacaaggccacattgactgcggacaaatcctccagcacagtctatttggagcttggtagattgacatctaaggactctgcggtctatttctgtgcaagacacggagcctactatagtaacgcctttgactactggggccaaggcaccactctcacagtctcctca

SEQ ID NO: 74 - DNA, 333 bp, synthetic construct
  source 1..333: mol_type = other DNA
aacattgtaatgacccaatctccagcttcattggctgtgtctctaggtcagagggccaccatctcctgcagagccagcgagagtgttgataatgatggcattagatttatgcactggtaccagcagaaaccaggacagccacccaaactcctcatctatcgtgcatccaacctagaatctgggatccctgccaggttcagtggcagtgggtctaggacagacttcaccctcactattaatcctgtggagactgatgatgttgcaacctattactgtcagcaaagtaataaggatccgtacacgttcggaggggggaccaagctggagctgaaa

SEQ ID NO: 75 - DNA, 348 bp, synthetic construct
  source 1..348: mol_type = other DNA
gaggtccagcttcagcagtctggagctgagctggtgaggcctgggtcctcagtgaagatgtcctgcaagacttctggatatactttcacaagctacggtataaactgggtgaagcagaggcctggacagggcctggaatggattggatatatttatattggaaatggttatgctgagtacaatgagaggttcaagggcaaggccacactgacttcagacacatcctccagcacagcctacatgcagctcagcagcctgacatctgaggactctgcaatctatttctgtgcactatactatccctggtttacttactggggccaggggactctggtcactgtctctgca

SEQ ID NO: 76 - DNA, 321 bp, synthetic construct
  source 1..321: mol_type = other DNA
gacatccagatgactcagtctccagcctccctttctgcatctgtgggagattctgtcaccatcacatgtcgagcaagtgagaatatttacagttatttagcatggtatcagcagaaacagggaaaatctcctcagctcctggtctataatgcaaaaaccttagctgaaggtgtgccatcaaggttcagtggcagtggatcaggcacacagttttctctgaagatcaacagcctgcagcctgaagattttgggaattattactgtcaacatcattatgatactccgttcacgttcggaggggggaccaagctggaaataaaa

SEQ ID NO: 77 - DNA, 363 bp, synthetic construct
  source 1..363: mol_type = other DNA
caggttcagctgcaccagtctggacctgagctggtgaagcctggggcttcagtgaagttgtcctgcaagacttctggctacaccttcacaagctacgatgtcttctgggtgaagcagaggcctggacagggacttgagtggattggatggatttatcctagagatagtagtactaaatacaatgagaagttcaagggcaaggccacattgactgtagacacatcctccagcacagcatacatggagctccacagcctgacatctgaggactctgcggtctatttctgtgcaaaagaggggtatgattatgacaagaggggctttgactactggggccaaggcaccactctcacagtctcctca

SEQ ID NO: 78 - DNA, 333 bp, synthetic construct
  source 1..333: mol_type = other DNA
gacattgtgctgacccaatctccagcttctttggctgtgtctctagggcagagggccatcatctcctgcaaggccagccaaagtgtcagttttgctggtactaatttaatgcactggtaccaacagagaccagggcagcaacccaaactcctcatctatcgtgcatccaacctagaacctggggttcctaccaggtttagtggcagtgggtctaggacagacttcaccctcaatatccatcctgtggaggaagatgatgctgcaacctattactgtcagcaaagtagggaatatccgtggacgttcggtggaggcaccaagctggaaatcaaa

SEQ ID NO: 79 - DNA, 345 bp, synthetic construct
  source 1..345: mol_type = other DNA
caggttcaactgcagcagtctggggctgagctggtgaggcctggggcttcagtgacgctgtcctgcaaggcttcgggctacacttttactgactatgaaatgcactgggtgaagcagacacctgtgcatggcctggaatggattggagctattgatcctgaaactggtggtactgcctacaatcagaggttcaagggcaaggccatactgactacagacaaatcctccatcacagcctacatggagctccgcagcctgacatctgaggactctgccgtctattactgtacaagatggaatgatggcgactactggggccaaggcaccactctcacagtctcctca

SEQ ID NO: 80 - DNA, 336 bp, synthetic construct
  source 1..336: mol_type = other DNA
gatgttgtgatgacccagactccactgtctttgtcggttaccattggacaaccagcctccatttcttgcaagtcaagtcagagcctcttatacagtaatggaaagacatatttgaattggtttcaacagaggcctggccagtctccaaagcgcctaatgtatcaggtgtccaaactggaccctggcatccctgacaggttcagtggcagtggatcagaaacagattttacacttaaaatcagcagagtggaggctgaagatttgggactttattactgcttgcaaggtacatattatccgtacacgttcggaggggggaccaagctggaaataaaa

SEQ ID NO: 81 - DNA, 360 bp, synthetic construct
  source 1..360: mol_type = other DNA
gaggtccagctgcagcagtctggacctgagctggtgaagcctggggcttcagtgaagatgtcctgcaaggcttctggttactcctttactggctactttatgaactgggtgaagcagagccatggaaagagccttgagtggattggacgtattaatccttacaatggtgatactttctacaaccagaagttcaagggcaaggccacattgactgtagacaaatcctctagcacagcccacatggagctccggagcctgacatctgaggactctgcactctattattgtgcaagcgaaggtgatggttactactggtacttcgatgtctggggcgcagggaccacggtcaccgtctcctca

SEQ ID NO: 82 - DNA, 333 bp, synthetic construct
  source 1..333: mol_type = other DNA
gacattgtgctgacccaatctccagcttctttggctgtgtctctagggcagagggccaccatctcctgcagagccagcgaaagtgttgataattatggcattagttttatgaactggttccaacagaaaccaggacagccacccaaactcctcatctatgctgcatccaaccaaggatccggggtccctgccaggtttagtggcagtgggtctgggacagacttcagcctcaacatccatcctatggaggaggatgatactgcaatgtatttctgtcagcaaagtaaggaggttcctcggacgttcggtggaggcaccaagctggaaatcaaa

SEQ ID NO: 83 - DNA, 345 bp, synthetic construct
  source 1..345: mol_type = other DNA
caggttcaactgcagcagtctggggctgagctggtgaggcctggggcttcagtgaagctgtcctgcaaggcttcgggctacacatttactgaccatgaaatgcactgggtgagacagacacctgtgcatggcctggaatggattggagttattgatcctgacactggtgatactacctacaatcagaaattcaagggcaaggccacactgactgcagacaaatcctccagcacagcctacatggacctccgcagcctgacatctgaggactctgccgtcttttactgtacacggtggactgggggggactactggggccatggcaccactctcacagtctcctca

SEQ ID NO: 84 - DNA, 336 bp, synthetic construct
  source 1..336: mol_type = other DNA
gatgctgtgatgacccagactccactgtctttgtcggttaccattggacaaccagcctctatctcttgcaagtcgagtcagagcctcttatatagtgatggaaagacatatttgaattggttccaacagaggccaggccagtctccaaagcgcctaatgtatcaggtgtccaaactggaccctggcatccctgacaggttcagtggcagtggatcagagacagattttacacttaaaatcagcagagtggaggctgaggatttgggagtttattactgcttgcaaggtacatattatccgtatacgttcggatcggggaccaagctggaaataaaa

SEQ ID NO: 85 - protein, 250 aa, Homo sapiens [sequence omitted]

SEQ ID NO: 86 - protein, 234 aa, Homo sapiens
MPASSPFLLAPKGPPGNMGGPVREPALSVALWLSWGAALGAVACAMALLTQQTELQSLRREVSRLQGTGGPSQNGEGYPWQSLPEQSSDALEAWENGERSRKRRAVLTQKQKNDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 87 - protein, 247 aa, Homo sapiens
MPASSPFLLAPKGPPGNMGGPVREPALSVALWLSWGAALGAVACAMALLTQQTELQSLRREVSRLQGTGGPSQNGEGYPWQSLPEQSSDALEAWENGERSRKRRAVLTQKQKKQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGL

SEQ ID NO: 88 - protein, 222 aa, Homo sapiens
MPASSPFLLAPKGPPGNMGGPVREPALSVALWLSWGAALGAVACAMALLTQQTELQSLRREVSRLQGTGGPSQNGEGYPWQSLPEQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 89 - protein, 330 aa, Homo sapiens
MAARRSQRRRGRRGEPGTALLVPLALGLGLALACLGLLLAVVSLGSRASLSAQEPAQEELVAEEDQDPSELNPQTEESQDPAPFLNRLVRPRRSAPKGRKTRARRAIAAHYEVHPRPGQDGAQAGVDGTVSGWEEARINSSSPLRYNRQIGEFIVTRAGLYYLYCQSSDALEAWENGERSRKRRAVLTQKQKKQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 90 - protein, 205 aa, Homo sapiens
MGGPVREPALSVALWLSWGAALGAVACAMALLTQQTELQSLRREVSRLQGTGGPSQNGEGYPWQSLPEQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 91 - protein, 241 aa, Mus musculus
MPASSPGHMGGSVREPALSVALWLSWGAVLGAVTCAVALLIQQTELQSLRREVSRLQRSGGPSQKQGERPWQSLWEQSPDVLEAWKDGAKSRRRRAVLTQKHKKKHSVLHLVPVNITSKADSDVTEVMWQPVLRRGRGLEAQGDIVRVWDTGIYLLYSQVLFHDVTFTMGQVVSREGQGRRETLFRCIRSMPSDPDRAYNSCYSAGVFHLHQGDIITVKIPRANAKLSLSPHGTFLGFVKL

SEQ ID NO: 92 - protein, 240 aa, Mus musculus
MPASSPGHMGGSVREPALSVALWLSWGAVLGAVTCAVALLIQQTELQSLRREVSRLQRSGGPSQKQGERPWQSLWEQSPDVLEAWKDGAKSRRRRAVLTQKHKKKHSVLHLVPVNITSKDSDVTEVMWQPVLRRGRGLEAQGDIVRVWDTGIYLLYSQVLFHDVTFTMGQVVSREGQGRRETLFRCIRSMPSDPDRAYNSCYSAGVFHLHQGDIITVKIPRANAKLSLSPHGTFLGFVKL

SEQ ID NO: 93 - protein, 7 aa, synthetic construct
GFSLTIY

SEQ ID NO: 94 - protein, 5 aa, synthetic construct
WSDGS

SEQ ID NO: 95 - protein, 10 aa, synthetic construct
NWVDQAWFAY

SEQ ID NO: 96 - protein, 11 aa, synthetic construct
RASKNIYSYLA

SEQ ID NO: 97 - protein, 7 aa, synthetic construct
NAKTLPE

SEQ ID NO: 98 - protein, 9 aa, synthetic construct
QHHYGTPLT

SEQ ID NO: 99 - protein, 5 aa, synthetic construct
IYDVH

SEQ ID NO: 100 - protein, 16 aa, synthetic construct
VIWSDGSTDYNAAFIS

SEQ ID NO: 101 - protein, 118 aa, synthetic construct
QVQLKESGPGLVAPSQSLSITCTVSGFSLTIYDVHWVRQSPGKGLEWLGVIWSDGSTDYNAAFISRLSISKDNSKSQVFFKMNSLQADDTAIYYCARNWVDQAWFAYWGQGTLVTVSA

SEQ ID NO: 102 - protein, 107 aa, synthetic construct
DIQMTQSPASLSASVGETITITCRASKNIYSYLAWYQQKQGKSPQLLVYNAKTLPEGVPSRFSGSGSGTQFSLKINSLQPEDFGSYYCQHHYGTPLTFGAGTKLELK

SEQ ID NO: 103 - protein, 7 aa, synthetic construct
GYIFTDY

SEQ ID NO: 104 - protein, 6 aa, synthetic construct
YPGSGN

SEQ ID NO: 105 - protein, 12 aa, synthetic construct
ESNYVGYYAMDY

SEQ ID NO: 106 - protein, 16 aa, synthetic construct
RSSQSVVNSNGNTYLE

SEQ ID NO: 107 - protein, 7 aa, synthetic construct
KVSNRFS

SEQ ID NO: 108 - protein, 9 aa, synthetic construct
FQGSHVPWT

SEQ ID NO: 109 - protein, 5 aa, synthetic construct
DYTIN

SEQ ID NO: 110 - protein, 17 aa, synthetic construct
WIYPGSGNRKYNDKFKG

SEQ ID NO: 111 - protein, 121 aa, synthetic construct
QVQLQQSGPELVKPGASVKLSCKAAGYIFTDYTINWVKQSPGQGLEWIGWIYPGSGNRKYNDKFKGKATMTADKSSSTAYMQLSSLTSEDSAVYFCARESNYVGYYAMDYWGQGTSVTVSS

SEQ ID NO: 112 - protein, 112 aa, synthetic construct
DVLMTQTPLSLPVSLGDQASISCRSSQSVVNSNGNTYLEWYLQKPGQSPNLLIYKVSNRFSGVPDRFSGSGSGTDFTLKISRVEAEDLGVYYCFQGSHVPWTFGGGTKLEIK

SEQ ID NO: 113 - protein, 7 aa, synthetic construct
GYTFTNY

SEQ ID NO: 114 - protein, 9 aa, synthetic construct
YPGGIGGGY

SEQ ID NO: 115 - protein, 9 aa, synthetic construct
SETGRAMDY

SEQ ID NO: 116 - protein, 11 aa, synthetic construct
KASQDINKYIA

SEQ ID NO: 117 - protein, 7 aa, synthetic construct
YTSTLKP

SEQ ID NO: 118 - protein, 8 aa, synthetic construct
LQYDNLNT

SEQ ID NO: 119 - protein, 5 aa, synthetic construct
NYWIG

SEQ ID NO: 120 - protein, 20 aa, synthetic construct
DIYPGGIGGGYTKYNEKFKG

SEQ ID NO: 121 - protein, 121 aa, synthetic construct
QVQLQQSGAELVRPGTSVKMSCKAAGYTFTNYWIGWVKQRPGHGLEWIGDIYPGGIGGGYTKYNEKFKGKATLTADTSSSTAYMQLGSLTSEDSAIYFCSRSETGRAMDYWGQGTSVTVSS

SEQ ID NO: 122 - protein, 106 aa, synthetic construct
DIQMTQSPSSLSASLGGKVTITCKASQDINKYIAWYQHKPGKGPRLLIHYTSTLKPGIPSRFSGSGSGRDYSFSISDLEPEDIATYYCLQYDNLNTFGGGTKLEIK

SEQ ID NO: 123 - protein, 7 aa, synthetic construct
GYSFTDY

SEQ ID NO: 124 - protein, 6 aa, synthetic construct
DPSNGG

SEQ ID NO: 125 - protein, 11 aa, synthetic construct
RDNYGSGTMDY

SEQ ID NO: 126 - protein, 11 aa, synthetic construct
KASQNVGTDVS

SEQ ID NO: 127 - protein, 7 aa, synthetic construct
WASNRFT

SEQ ID NO: 128 - protein, 9 aa, synthetic construct
EQYSIYPLT

SEQ ID NO: 129 - protein, 5 aa, synthetic construct
DYNIY

SEQ ID NO: 130 - protein, 17 aa, synthetic construct
YIDPSNGGPGYNQKFRG

SEQ ID NO: 131 - protein, 120 aa, synthetic construct
EIQLQQSGPELVKPGASVKVSCKASGYSFTDYNIYWVKQSHGKSLEWIGYIDPSNGGPGYNQKFRGKATLTVDKSSSTAFLHLNSLTSEDSAVYYCARRDNYGSGTMDYWGQGTSVTVSS

SEQ ID NO: 132 - protein, 107 aa, synthetic construct
DIVMTQSQKFMSTSVGDRVSITCKASQNVGTDVSWYQQKPGKSPKPLIYWASNRFTGVPDRFIGSGSGTDFTLTISNVQSEDLADYFCEQYSIYPLTFGAGTKLELK

SEQ ID NO: 133 - protein, 7 aa, synthetic construct
GYSFTDD

SEQ ID NO: 134 - protein, 6 aa, synthetic construct
DPLNGG

SEQ ID NO: 135 - protein, 11 aa, synthetic construct
RDNYATGTMDY

SEQ ID NO: 136 - protein, 11 aa, synthetic construct
KASKNVGTDVS

SEQ ID NO: 137 - protein, 9 aa, synthetic construct
EQYSSYPLT

SEQ ID NO: 138 - protein, 5 aa, synthetic construct
DDNMY

SEQ ID NO: 139 - protein, 17 aa, synthetic construct
YIDPLNGGTGYNQKFKG

SEQ ID NO: 140 - protein, 120 aa, synthetic construct
EIQLQQSGPELVKPGASVKVSCKASGYSFTDDNMYWVKQSHGKSLEWIGYIDPLNGGTGYNQKFKGKATLTVDKSSSTAFLHLNSLTSEDSAVYYCARRDNYATGTMDYWGQGTSVTVSS

SEQ ID NO: 141 - protein, 107 aa, synthetic construct
DIVMTQSQKFMSTSVGDRVSITCKASKNVGTDVSWYQQKPGKSPKPLIYWASNRFTGVPDRFTGSGSGTDFTLTINNVQSEDLADYFCEQYSSYPLTFGAGTKLELK

SEQ ID NO: 142 - protein, 6 aa, synthetic construct
YPINGY

SEQ ID NO: 143 - protein, 11 aa, synthetic construct
DSNYVGWYFDV

SEQ ID NO: 144 - protein, 16 aa, synthetic construct
RSSQSLVHSNGNTYLH

SEQ ID NO: 145 - protein, 9 aa, synthetic construct
SQSTHVPRT

SEQ ID NO: 146 - protein, 15 aa, synthetic construct
RASKSVSTSGYSYMH

SEQ ID NO: 147 - protein, 7 aa, synthetic construct
FTSDLEP

SEQ ID NO: 148 - protein, 9 aa, synthetic construct
QHSRELPYP

SEQ ID NO: 149 - protein, 5 aa, synthetic construct
DYNMD

SEQ ID NO: 150 - protein, 17 aa, synthetic construct
NIYPINGYTGYNQRFKN

SEQ ID NO: 151 - protein, 120 aa, synthetic construct
EVQLQQSGPELVKPGASVKISCKASGYTFTDYNMDWVKQSHGKSLEWIGNIYPINGYTGYNQRFKNKATLTVDKSSSTAYMELHSLTSEDSAVYYCARDSNYVGWYFDVWGAGTTVTVSS

SEQ ID NO: 152 - protein, 112 aa, synthetic construct
DVVMTQTPLSLPVSLGDQASISCRSSQSLVHSNGNTYLHWYLQKPGQSPKLLIYKVSNRFSGVPDRFSGSGSGTDFTFKISRVEAEDLGVYFCSQSTHVPRTFGGGTKLEIK

SEQ ID NO: 153 - protein, 111 aa, synthetic construct
DIVLTQSPASLTVSLGQRATFSCRASKSVSTSGYSYMHWYQQKPGQPPKLLIYFTSDLEPGVPARFTGSGSGTDFTLNIHPVEEEDAATYYCQHSRELPYPFGGGTKLEIK

SEQ ID NO: 154 - protein, 7 aa, synthetic construct
GYTFADY

SEQ ID NO: 155 - protein, 6 aa, synthetic construct
FPGSGS

SEQ ID NO: 156 - protein, 9 aa, synthetic construct
GDSGRAMDY

SEQ ID NO: 157 - protein, 7 aa, synthetic construct
YTSTLQS

SEQ ID NO: 158 - protein, 8 aa, synthetic construct
LQYDNLLT

SEQ ID NO: 159 - protein, 5 aa, synthetic construct
DYYIN

SEQ ID NO: 160 - protein, 17 aa, synthetic construct
WIFPGSGSTYYNEKFKG

SEQ ID NO: 161 - protein, 118 aa, synthetic construct
QVQLQQSGPELVKPGASVKISCKASGYTFADYYINWVKQRPGQGLEWIGWIFPGSGSTYYNEKFKGKATLTVDKSSSTAYMLLSSLTSEDSAVYFCARGDSGRAMDYWGQGTSVTVSS

SEQ ID NO: 162 - protein, 106 aa, synthetic construct
DIQMTQSPSSLSASLGGKVTITCKASQDINKYIAWYQHKPGKGPRLLIHYTSTLQSGIPSRFSGSGSGRDYSFSISNLEPEDNATYYCLQYDNLLTFGAGTKLELK

SEQ ID NO: 163 - protein, 7 aa, synthetic construct
GFSLTDY

SEQ ID NO: 164 - protein, 5 aa, synthetic construct
WNDGS

SEQ ID NO: 165 - protein, 10 aa, synthetic construct
NWYGGYWFAY

SEQ ID NO: 166 - protein, 11 aa, synthetic construct
RSSENIYSYLA

SEQ ID NO: 167 - protein, 7 aa, synthetic construct
NANALAE

SEQ ID NO: 168 - protein, 9 aa, synthetic construct
QHHYGTPFT

SEQ ID NO: 169 - protein, 5 aa, synthetic construct
DYDVH

SEQ ID NO: 170 - protein, 16 aa, synthetic construct
VIWNDGSTDYNTAFIS

SEQ ID NO: 171 - protein, 118 aa, synthetic construct
QAHLKESGPGLVAPSQSLSITCTVSGFSLTDYDVHWVRQSPGKGLEWLGVIWNDGSTDYNTAFISRLTISKDNSKSQVFFKMNSLQADDTAIYYCARNWYGGYWFAYWGQGTLVTVSA

SEQ ID NO: 172 - protein, 107 aa, synthetic construct
DIQMTQSPASLSASAGETVTITCRSSENIYSYLAWYQQKQGKSPQLLVYNANALAEGVPSRFSGSGSVTQFSLKINSLQPEDFGSYYCQHHYGTPFTFGSGTKLEIK

SEQ ID NO: 173 - DNA, 354 bp, synthetic construct
  source 1..354: mol_type = other DNA
caggtgcagctgaaggagtcaggacctggcctggtggcgccctcacagagcctgtccatcacctgcacagtctctggtttctcattaaccatctatgatgtacactgggttcgccagtctccaggaaagggtctggagtggctgggagtgatatggagtgatggaagcacagactataatgcagctttcatatctagactgagcatcagcaaggacaactccaagagccaagttttctttaaaatgaacagtctgcaagctgatgacacagccatatactactgtgccagaaattgggtcgaccaggcctggtttgcttactggggccaagggactctggtcactgtctctgca

SEQ ID NO: 174 - DNA, 321 bp, synthetic construct
  source 1..321: mol_type = other DNA
gacatccagatgactcagtctccagcctccctatctgcatctgtgggagaaactatcaccatcacatgtcgagcaagtaagaatatttacagttatttagcatggtatcagcagaaacagggaaaatctcctcagctcctggtctataatgcaaaaaccttaccagaaggtgtgccatcaaggttcagtggcagtggatcaggcacacagttttctctgaagatcaacagcctgcagcctgaagattttgggagttattactgtcaacatcattatggtactccgctcacgttcggtgctgggaccaagctggagctgaaa

SEQ ID NO: 175 - DNA, 363 bp, synthetic construct
  source 1..363: mol_type = other DNA
caggtccaactgcagcagtctggacctgagctggtgaagcctggagcttcagtgaagctgtcctgcaaggctgctggctacatcttcactgactatactataaactgggtgaagcagagtcctggacagggacttgagtggattggatggatttatcctggaagtggtaatcgtaaatacaatgacaagttcaagggcaaggccacaatgactgcagacaaatcctccagcacagcctacatgcagctcagcagcctgacctctgaggattctgcggtctatttctgtgcaagagagagtaactacgtggggtactatgctatggactattggggtcaaggaacctcagtcaccgtctcctca

SEQ ID NO: 176 - DNA, 336 bp, synthetic construct
  source 1..336: mol_type = other DNA
gatgttttgatgacccaaactccactctccctgcctgtcagtcttggagatcaagcctccatctcttgcagatctagtcagagcgttgtaaatagtaatggaaacacctatttagaatggtacctgcagaaaccaggccagtctccaaatctcctgatctacaaagtttccaatcgattttctggggtcccagacaggttcagtggcagtggatcggggacagatttcacactcaagatcagcagagtggaggctgaggatctgggagtttattactgttttcaaggttcacatgttccgtggacgttcggtggaggcaccaagctggaaatcaaa

SEQ ID NO: 177 - DNA, 363 bp, synthetic construct
  source 1..363: mol_type = other DNA
caggtccagctgcagcagtctggagctgagctggtaaggcctgggacttcagtgaagatgtcctgcaaggctgctggatacaccttcacaaactactggataggttgggtaaagcagaggcctggacatggccttgagtggattggagatatttaccctggaggtataggaggtggttatactaagtacaatgagaagttcaagggcaaggccacactgactgcagacacatcctccagcacagcctacatgcagctcggcagcctgacatctgaggactctgccatctatttctgttcaagatcggaaactggacgggctatggactactggggtcaaggaacctcagtcaccgtctcctca

SEQ ID NO: 178 - DNA, 318 bp, synthetic construct
  source 1..318: mol_type = other DNA
gacatccagatgacacagtctccatcctcactgtctgcatctctgggaggcaaagtcaccatcacttgcaaggcaagccaagacattaataagtatatagcttggtaccaacacaagcctggaaaaggtcctaggctgctcatacattacacatctacattaaagccaggcatcccatcaaggttcagtggaagtgggtctgggagagattattccttcagcatcagtgacctggagcctgaagatattgcaacttattattgtctacagtatgataatctgaacacgttcggaggggggaccaagctggaaataaaa

SEQ ID NO: 179 - DNA, 360 bp, synthetic construct
  source 1..360: mol_type = other DNA
gagatccagctgcagcagtctggacctgagctggtgaagcctggggcttcagtgaaggtgtcctgcaaggcttctggttattcattcactgactacaacatctactgggtgaagcagagccatggaaagagccttgagtggattggatatattgatccttccaatggtggtcctggctacaaccagaagttcaggggcaaggccacattgactgttgacaagtcctccagcacagccttcctgcatctcaacagcctgacatctgaggactctgcagtctattactgtgcaagaagggacaactacggctcggggactatggactactggggtcaaggaacctcagtcaccgtctcctca

SEQ ID NO: 180 - DNA, 321 bp, synthetic construct
  source 1..321: mol_type = other DNA
gacattgtgatgacccagtctcaaaaattcatgtccacatcagtaggagacagggtcagcatcacctgtaaggccagtcagaatgtgggtactgatgtatcctggtatcaacagaaaccagggaaatctcctaaaccactgatttactgggcatcaaaccggttcactggagtccctgatcgcttcataggtagtggatctgggacagatttcactctcaccatcagcaatgtgcagtctgaagacttggcagattatttctgtgagcaatatagcatctatccgctcacgttcggtgctgggaccaagctggagctgaaa

SEQ ID NO: 181 - DNA, 360 bp, synthetic construct
  source 1..360: mol_type = other DNA
gagatccagctgcagcagtctggacctgagctggtgaagcctggggcgtcagtgaaggtatcctgcaaggcttctggttactcattcactgacgacaacatgtactgggtgaagcagagccatggaaagagccttgagtggattggatatattgatcctctcaatggtggtactggctacaaccagaaattcaagggcaaggccacactgactgttgacaagtcctccagcacagccttcctgcatctcaacagcctgacatctgaggactctgcagtctattactgtgcaagaagggacaactacgccacggggactatggactactggggtcaaggaacctcagtcaccgtctcctca

SEQ ID NO: 182 - DNA, 321 bp, synthetic construct
  source 1..321: mol_type = other DNA
gacattgtgatgacccagtctcaaaaattcatgtccacatcagtaggagacagggtcagcatcacctgcaaggccagtaagaatgtgggtactgatgtatcctggtatcaacagaaaccagggaaatctcctaaaccactgatttactgggcatcaaaccggttcactggagtccctgatcgcttcacaggcagtggatctgggacagatttcactctcaccatcaacaatgtgcagtctgaagacttggcagattatttctgtgagcaatatagcagctatccgctcacgttcggtgctgggaccaagctggagctgaaa

SEQ ID NO: 183 - DNA, 360 bp, synthetic construct
  source 1..360: mol_type = other DNA
gaggtccagctgcagcagtctggccctgagctggtgaagcctggggcttcagtgaagatatcctgcaaggcttctggatacacattcactgactacaacatggactgggtgaagcagagccatggaaagagccttgagtggattggaaatatttatcctatcaatggttatactggctacaaccagaggttcaagaacaaggccacattgactgtagacaagtcctccagcacagcctacatggaactccacagcctgacatctgaggactctgcggtctattactgcgcaagagatagtaactacgttggctggtacttcgatgtctggggcgcagggaccacggtcaccgtctcctca

SEQ ID NO: 184 - DNA, 336 bp, synthetic construct
  source 1..336: mol_type = other DNA
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacattcaagatcagcagagtggaggctgaggatctgggagtttatttctgctctcaaagtacacatgttcctcggacgttcggtggaggcaccaagctggaaatcaaa

SEQ ID NO: 185 - DNA, 333 bp, synthetic construct
  source 1..333: mol_type = other DNA
gacattgtgctgacacagtctcctgcttccttaactgtatctctggggcagagggccaccttctcatgcagggccagcaaaagtgtcagtacatctggctatagttatatgcactggtaccaacagaaaccaggacagccacccaaactcctcatctattttacatccgacctagaacctggggtccctgccaggttcactggcagtgggtctgggacagacttcaccctcaacatccatcctgtggaggaggaggatgctgcaacctattactgtcagcacagtagggagcttccgtaccccttcggaggggggaccaagttggaaataaaa

SEQ ID NO: 186 - DNA, 354 bp, synthetic construct
  source 1..354: mol_type = other DNA
caggtccagctacagcagtctggacctgagctggtgaagcctggggcttcagtgaagatatcctgcaaggcttctggctacaccttcgctgactactatataaactgggtgaagcagaggcctggacagggacttgagtggattggatggatttttcctggaagtggtagtacttactacaatgagaagttcaagggcaaggccacacttactgtagacaaatcctccagcacagcctacatgttgctcagcagcctgacctctgaggactctgcggtctatttctgtgcaagaggggactccggtagggctatggactactggggtcaaggaacctcagtcaccgtctcctca

SEQ ID NO: 187 - DNA, 318 bp, synthetic construct
  source 1..318: mol_type = other DNA
gacatccagatgacacagtctccatcctcactgtctgcatctctgggaggcaaagtcaccatcacttgcaaggcaagccaagacattaacaaatatatagcttggtaccaacacaagcctggaaaaggtcctaggctgctcatacattacacatctacattacagtcaggcatcccatcaaggttcagtggaagtgggtctgggagagattattccttcagcatcagcaacctggagcctgaagataatgcaacttattattgtctacagtatgataatcttctcacgttcggtgctgggaccaagctggagctgaaa

SEQ ID NO: 188 - DNA, 354 bp, synthetic construct
  source 1..354: mol_type = other DNA
caggcgcacctgaaggagtcaggacctggcctggtggcgccctcacagagcctgtccatcacctgcacagtctctggtttctcattaaccgactatgatgtacactgggttcgccagtctccaggaaagggtctggagtggctgggagtgatatggaatgatggaagcacagactataatacagctttcatatctagactgaccatcagcaaggacaactccaagagccaagttttctttaaaatgaacagtctgcaagctgatgacacagccatatactactgtgccagaaattggtatggtggctactggtttgcttactggggccaagggactctggtcactgtctctgca

SEQ ID NO: 189 - DNA, 321 bp, synthetic construct
  source 1..321: mol_type = other DNA
gacatccagatgactcagtctccagcctccctatctgcatctgcgggagaaactgtcaccatcacatgtcgatcaagtgagaatatttacagttatttagcatggtatcagcagaaacagggaaaatctcctcagctcctagtctataatgcaaatgccttagcagaaggtgtgccatcgaggttcagtggcagtggatcagtcacacagttttctctgaagatcaacagcctgcagcctgaagattttgggagttattactgtcaacatcattatggtactccattcacgttcggctcggggacaaagttggaaataaaa

SEQ ID NO: 190 - protein, 120 aa, synthetic construct
QVQLQESGPGLVKPSQTLSLTCTVSGYSITSGYYWNWIRQHPGKGLEWIGYISYDGYNNYNPSLKNRVTISVDTSKNQFSLKLSSVTAADTAVYYCARYYDYEDWYFGVWGQGTMVTVSS

SEQ ID NO: 191 - protein, 120 aa, synthetic construct
QVQLQESGPGLVKPSQTLSLTCTVSGYSITSGYYWSWIRQHPGKGLEWIGYISYDGYTYYNPSLKSRVTISVDTSKNQFSLKLSSVTAADTAVYYCARYYDYEDWYFGVWGQGTMVTVSS

SEQ ID NO: 192 - protein, 120 aa, synthetic construct
QVQLQESGPGLVKPSQTLSLTCTVSGYSITSGYYWNWIRQHPGKGLEWIGYISYDGYNNYNPSLKSRVTISRDTSKNQFSLKLSSVTAADTAVYYCARYYDYEDWYFGVWGQGTMVTVSS

SEQ ID NO: 193 - protein, 120 aa, synthetic construct
QVQLQQWGAGLLKPSETLSLTCAVYGYSITSGYYWNWIRQPPGKGLEWIGYISYDGYNNYNPSLKNRVTISVDTSKNQFSLKLSSVTAADTAVYYCANYYDYEDWYFGVWGQGTTVTVSS

SEQ ID NO: 194 - protein, 120 aa, synthetic construct
QVQLQQWGAGLVKPSETLSLTCAVYGYSITSGYYWNWIRQPPGKGLEWIGYISYDGYNNYNPSLKNRVTISRDTSKNQFSLKLSSVTAADTAVYYCANYYDYEDWYFGVWGQGTTVTVSS

SEQ ID NO: 195 - protein, 120 aa, synthetic construct
QVQLQESGPGLVKPSETLSLTCTVSGYSITSGYYWNWIRQPPGKGLEWIGYISYDGYNNYNPSLKNRVTISVDTSKNQFSLKLSSVTAADTAVYYCANYYDYEDWYFGVWGQGTTVTVSS

SEQ ID NO: 196 - protein, 120 aa, synthetic construct
QVQLQESGPGLVKPSETLSLTCTVSGYSITSGYYWNWIRQPPGKGLEWIGYISYDGYNNYNPSLKNRVTISRDTSKNQFSLKLSSVTAADTAVYYCANYYDYEDWYFGVWGQGTTVTVSS

SEQ ID NO: 197 - protein, 120 aa, synthetic construct
QVQLQESGPGLMKPSETLSLTCSVSGYSITSGYYWSWIRKPPGKGLEYIGYVSYDGSTYYNPSLKSRVTISVDTSKNRFSLKLNSVTAADTAVYYCANYYDYEDWYFGYWGQGILVTVSS

SEQ ID NO: 198 - protein, 120 aa, synthetic construct
QVQLQESGPGLMKPSETLSLTCSVSGYSITSGYYWNWIRKPPGKGLEWIGYISYDGYNNYNPSLKSRVTISRDTSKNRFSLKLNSVTAADTAVYYCANYYDYEDWYFGVWGQGILVTVSS

SEQ ID NO: 199 - protein, 120 aa, synthetic construct
EVQLVESGGGLVQPGGSLRLSCAVSGYSITSGYYWNWIRQAPGKGLEWVASISYDGYNNYNPSVKGRITISRDDSKNTFYLQMNSLRAEDTAVYYCANYYDYEDWYFGVWGQGTLVTVSS

SEQ ID NO: 200 - protein, 120 aa, synthetic construct
EVQLVESGGGLVQPGGSLRLSCAVSGYSITSGYYWNWIRQAPGKGLEWVAYISYDGYNNYNPSVKGRITISRDTSKNTFYLQMNSLRAEDTAVYYCANYYDYEDWYFGVWGQGTLVTVSS

SEQ ID NO: 201 - protein, 120 aa, synthetic construct
QVQLVESGGGVVQPGRSLRLSCAASGYSITSGYYWNWVRQAPGKGLEWVAYISYDGYNNYNPSVKGRFTISRDNSKNTLYLQMNSLRAEDTAVYYCANYYDYEDWYFGVWGQGTMVTVSS

SEQ ID NO: 202 - protein, 111 aa, synthetic construct
EIVLTQSPATLSLSPGERATLSCRASESVSIIGTNSIHWYQQKPGQAPRLLIYHASNLETGIPARFSGSGPGTDFTLTISSLEPEDFAVYYCLQSRKIPYTFGQGTKLEIK

SEQ ID NO: 203 - protein, 111 aa, synthetic construct
DIVLTQSPASLAVSPGQRATITCRASESVSIIGTNSIHWYQQKPGQPPKLLIYHASNLETGVPARFSGSGSGTDFTLTINPVEANDTANYYCLQSRKIPYTFGGGTKLEIK

SEQ ID NO: 204 - protein, 111 aa, synthetic construct
EIVMTQSPATLSVSPGERATLSCRASESVSIIGTNSLHWYQQKPGQAPRLLIYHASQSISGIPARFSGSGSGTEFTLTISSLQSEDFAVYYCQQSRKIPYTFGGGTKVEIK

SEQ ID NO: 205 - protein, 111 aa, synthetic construct
EIVMTQSPATLSVSPGERATLSCRASESVSIIGTNSIHWYQQKPGQAPRLLIYHASNLETGIPARFSGSGSRTEFTLTISSLQSEDFAVYYCLQSRKIPYTFGGGTKVEIK

SEQ ID NO: 206 - protein, 111 aa, synthetic construct
DIQLTQSPSSLSASVGDRVTITCRASESVSIIGTNSMNWYQQKPGKAPKLLIYHASYLESGVPSRFSGSGSGTDFTLTISSLQPEDFATYYCLQSRKIPYTFGQGTKVEIK

SEQ ID NO: 207 - protein, 111 aa, synthetic construct
DIQLTQSPSSLSASVGDRVTITCRASESVSIIGTNSMHWYQQKPGKAPKLLIYHASNLESGVPSRFSGSGSRTDFTLTISSLQPEDFATYYCLQSRKIPYTFGQGTKVEIK

SEQ ID NO: 208 - protein, 111 aa, synthetic construct
DIQMTQSPSSLSASVGDRVTITCRASESVSIIGTNSMHWYQQKPGKAPKLLIYHASNLESGVPSRFSGSGSGTDFTFTISSLQPEDIATYYCLQSRKIPYTFGQGTKVEIK

SEQ ID NO: 209 - protein, 120 aa, synthetic construct
QVQLVQSGAEVKKPGASVKVSCKASGYTFTDYTIHWVRQAPGQGLEWMGWIYPLRGSINYNEKFKDRVTSTRDTSISTAYMELSRLRSDDTVVYYCARHGAYYSNAFDYWGQGTLVTVSS

SEQ ID NO: 210 - protein, 120 aa, synthetic construct
QVQLVQSGAEVKKPGASVKVSCKASGYTFTDYYMHWVRQAPGQGLEWMGRIYPLRGSTNYAQKFQGRVTSTRDTSISTAYMELSRLRSDDTVVYYCARHGAYYSNAFDYWGQGTLVTVSS

SEQ ID NO: 211 - protein, 120 aa, synthetic construct
QVQLVQSGAEVKKPGASVKVSCKASGYTFTDYTIHWVRQAPGQGLEWMGWIYPLRGSINYNEKFKDRVTMTADTSSSTAYMELSRLRSDDTVVYYCARHGAYYSNAFDYWGQGTLVTVSS

SEQ ID NO: 212 - protein, 120 aa, synthetic construct
QVQLVQSGAEVKKPGASVKVSCEASGYTFTDYTIHWVRQAPGKGLEWMGWIYPLRGSINYNEKFKDRVTMTADTSTDTAYMELSSLRSKDTAVYYCARHGAYYSNAFDYWGQGTLVTVSS

SEQ ID NO: 213 - protein, 120 aa, synthetic construct
QVQLVQSGAEVVKPGASVKLSCKASGYTFTDYTMYWVKQAPGQGLEWIGEIYPLRGSINFNEKFKSKATLTVDKSASTAYMELSSLRSEDTAVYYCARHGAYYSNAFDYWGQGTLVTVSS

SEQ ID NO: 214 - protein, 120 aa, synthetic construct
QVQLVQSGAEVVKPGASVKLSCKASGYTFTDYTMHWVKQAPGQGLEWIGEIYPLRGSINFNEKFKSKATLTVDKSASTAYMELSSLRSEDTAVYYCARHGAYYSNAFDYWGQGTLVTVSS

SEQ ID NO: 215 - protein, 111 aa, synthetic construct
EIVLTQSPATLSLSPGERATLSCRASESVDNDGIRFMHWYQQKPGQAPRLLIYRASNLESGIPARFSGSGPGTDFTLTISSLEPEDFAVYYCQQSNKDPYTFGQGTKLEIK

SEQ ID NO: 216 - protein, 111 aa, synthetic construct
EIVLTQSPATLSLSPGERATLSCRASESVDNDGIRFMHWYQQKPGQAPRLLIYRASNLESGIPARFSGSGPGTDFTLTISSLEPEDVAVYYCQQSNKDPYTFGQGTKLEIK

SEQ ID NO: 217 - protein, 111 aa, synthetic construct
DIVMTQSPASLAVSLGERATINCRASESVDNDGIRFMHWYQQKPGQPPKLLIYRASNLESGVPDRFSGSGSRTDFTLTISSLQAEDVAVYYCQQSNKDPYTFGGGTKVEIK

SEQ ID NO: 218 - protein, 111 aa, synthetic construct
DIVMTQSPASLAVSLGERATINCRASESVDNDGIRFMHWYQQKPGQPPKLLIYRASNLESGVPDRFSGSGSRTDFTLTINSLQAEDVAVYYCQQSNKDPYTFGGGTKVELK

SEQ ID NO: 219 - protein, 111 aa, synthetic construct
DIVLTQSPATLSVSPGERATISCRASESVDNDGIRFMHWYQQKPGQPPKLLIYRASNLESGVPARFSGSGSRTDFTLTISSVEPEDFATYYCQHSWEIPPTFGGGTKLEIK

SEQ ID NO: 220 - protein, 118 aa, synthetic construct
QVQLVESGGGVVQPGRSLRLSCAASGFSLTIYDVHWVRQAPGKGLEWVAVIWSDGSTDYNAAFISRFTISRDNSKNTLYLQMNSLRAEDTAVYYCARNWVDQAWFAYWGQGTLVTVSS

SEQ ID NO: 221 - protein, 118 aa, synthetic construct
QVQLVESGGGVVQPGRSLRLSCAASGFSLTIYDVHWVRQAPGKGLEWVGVIWSDGSTDYNAAFISRFTISKDNSKNTLYLQMNSLRAEDTAVYYCARNWVDQAWFAYWGQGTLVTVSS

SEQ ID NO: 222 - protein, 118 aa, synthetic construct
QVQLVESGGGVVQPGRSLRLSCAASGFSLTIYDVHWVRQAPGKGLEWVAVIWSDGSTDYADSVKGRFTISKDNSKNTLYLQMNSLRAEDTAVYYCARNWVDQAWFAYWGQGTLVTVSS

SEQ ID NO: 223 - protein, 118 aa, synthetic construct
QVQLVESGGGVVQPGRSLRLSCAVSGFSLTIYDVHWVRQAPGKGLEWVGVIWSDGSTDYADSVKGRFTISKDNSKNTVYLQMNSLRAEDTAVYYCARNWVDQAWFAYWGQGTLVTVSS

SEQ ID NO: 224 - protein, 118 aa, synthetic construct
QVQLQESGPGLVKPSETLSLTCTVSGFSLTIYDVHWIRQPPGKGLEWIGVIWSDGSTDYNAAFISRVTISVDTSKNQFSLKLSSVTAADTAVYYCARNWVDQAWFAYWGQGTLVTVSS

SEQ ID NO: 225 - protein, 118 aa, synthetic construct
QVQLQESGPGLVKPSETLSLTCTVSGFSLTIYDVHWVRQPPGKGLEWIGVIWSDGSTDYNPSLKSRVTISKDTSKNQVSLKLSSVTAADTAVYYCARNWVDQAWFAYWGQGTLVTVSS

SEQ ID NO: 226 - protein, 118 aa, synthetic construct
QVQLQESGPGLMKPSETLSLTCSVSGDSITIYDWHWIRQPPGKGLEWIGVVWSDGSTDYNPSLKSRVTISVDTSKNRFSLKLSSVTAADTAVYYCARNWVDQAWFAYWGQGTLVTVSS

SEQ ID NO: 227 - protein, 118 aa, synthetic construct
QVQLQESGPGLVKPSETLSLTCTVSGFSLTIYDVHWIRQPPGKGLEWIGVIWSDGSTDYNPSLKSRVTISKDNSKNQFSLKLSSVTAADTAVYYCARNWVDQAWFAYWGQGTLVTVSS

SEQ ID NO: 228 - protein, 107 aa, synthetic construct
DIQMTQSPSSLSASVGDRVTITCRASKNIYSYLAWYQQKPGKAPKLLIYNAKTLPEGVPSRFSGSGSGTDFTLTISSLQPEDFATYYCQHHYGTPLTFGQGTKLEIK

SEQ ID NO: 229 - protein, 107 aa, synthetic construct
DIQMTQSPSSLSASVGDRVTITCRASKNIYSYLAWYQQKPGKAPKLLVYNAKTLPEGVPSRFSGSGSGTDFTLTISSLQPEDFATYYCQHHYGTPLTFGQGTKLEIK

SEQ ID NO: 230 - protein, 107 aa, synthetic construct
EIVLTQSPATLSLSPGERATLSCRASKNIYSYLAWYQQKPGQAPRLLIYNAKTRATGIPARFSGSGSGTDFTLTISSLEPEDFAVYYCQHHYGTPLTFGQGTKLEIK

SEQ ID NO: 231 - protein, 107 aa, synthetic construct
EIVLTQSPATLSLSPGERATLSCRASKNIYSYLAWYQQKPGQAPRLLVYNAKTLPEGIPARFSGSGSGTDFTLTISSLEPEDFAVYYCQHHYGTPLTFGQGTKLEIK

SEQ ID NO: 232 - protein, 107 aa, synthetic construct
EIVMTQSPATLSVSPGERATLSCRASKNIYSYLAWYQQKPGQAPRLLIYNAKTRATGIPARFSGSGSGTEFTLTISSLQSEDFAVYYCQHHYGTPLTFGGGTKVEIK

SEQ ID NO: 233 - protein, 107 aa, synthetic construct
DIQLTQSPSFLSASVGDRVTITCRASKNIYSYLAWYQQKPGKAPKLLIYNAKSLQSGVPSRFSGSGSGTEFTLTISSLQPEDFATYYCQHHYGTPLTFGGGTKLEIK

SEQ ID NO: 234 - protein, 107 aa, synthetic construct; identical to SEQ ID NO: 233

SEQ ID NO: 235 - protein, 118 aa, synthetic construct
QLQLQESGSGLVKPSQTLSLTCAVSGFSLTDYDVHWVRQPPGKGLEWIGVIWNDGSTDYNPSLISRVTISKDNSKNQVSLKLSSVTAADTAVYYCARNWYGGYWFAYWGQGTLVTVSS

SEQ ID NO: 236 - protein, 118 aa, synthetic construct
QLQLQESGSGLVKPSQTLSLTCAVSGGSITDYDWHWVRQPPGKGLEWIGVIWNDGSTDYNPSLISRVTISVDNSKNQFSLKLSSVTAADTAVYYCARNWYGGYWFAYWGQGTLVTVSS

SEQ ID NO: 237 - protein, 118 aa, synthetic construct
QVQLVESGGGVVQPGRSLRLSCAASGFSFTDYDMHWVRQAPGKGLEWVAVIWNDGSTDYATSVIGRFTISRDNSKNTLYLQMNSLRAEDTAVYYCARNWYGGYWFAYWGQGTLVTVSS

SEQ ID NO: 238 - protein, 118 aa, synthetic construct
QVQLVESGGGVVQPGRSLRLSCAASGFSFTDYDMHWVRQAPGKGLEWVGVIWNDGSTDYATSVIGRFTISRDNSKNTLYLQMNSLRAEDTAVYYCARNWYGGYWFAYWGQGTLVTVSS

SEQ ID NO: 239 - protein, 118 aa, synthetic construct
QVQLQESGPGLMKPSETLSLTCSVSGGSITDYDWHWIRQPPGKGLEWIGVVWNDGSTDYNPSLKSRVTISVDTSKNRFSLKLNSVTAADTAVYYCARNWYGGYWFAYWGQGILVTVSS

SEQ ID NO: 240 - protein, 118 aa, synthetic construct
QVTLKESGPALVKPTQTLTLTCTFSGFSLTDYDVHWIRQPPGKALEWLAVIWNDGSTDYSPSLKSRLTITKDTSKNQVVLTMTNMDPVDTATYYCARNWYGGYWFAYWGQGTLVTVSS

SEQ ID NO: 241 - protein, 107 aa, synthetic construct
DIQMTQSPSSLSASVGDRVTITCRSSENIYSYLAWYQQKPGKAPKLLVYNANALAEGVPSRFSGSGSVTDFTLTISSLQPEDFATYYCQHHYGTPFTFGQGTKLEIK

SEQ ID NO: 242 - protein, 107 aa, synthetic construct
DIQMTQSPSTLSASVGDRVTITCRSSENIYSYLAWYQQKPGKAPKLLVYNANALAEGVPSRFSGSGSVTEFTLTISSLQPDDFATYYCQHHYGTPFTFGQGTKLEIK

SEQ ID NO: 243 - protein, 107 aa, synthetic construct
EIVMTQSPATLSVSPGERATLSCRASENIYSYLAWYQQKPGQAPRLLIYNANASAEGIPARFSGSGSGTEFTLTISSLQSEDFAVYYCQQHYGTPFTFGGGTKVEIK

SEQ ID NO: 244 - protein, 107 aa, synthetic construct
DIQMTQSPSSLSASVGDRVTITCRASENIYSYLAWYQQKPGKAPKLLLYNANRLESGVPSRFSGSGSGTDFTLTISSLQPEDFASYYCQHHYGTPFTFGSGTKLEIK

SEQ ID NO: 245 - protein, 107 aa, synthetic construct
DIQMTQSPSSLSASVGDRVTITCRASENIYSYLAWYQQKPGKAPKLLLYNANRLESGVPSRFSGSGSGTDYTLTISSLQPEDFASYYCQHHYGTPFTFGSGTKLEIK

SEQ ID NO: 246 - protein, 121 aa, synthetic construct
QVQLVQSGAEVKKPGASVKVSCKASGYTFTNYWIGWVRQAPGQGLEWMGDIYPGGIGGGYTKYNEKFKGRVTMTADTSTSTAYMELRSLRSDDTAVYYCSRSETGRAMDYWGQGTLVTVSS

SEQ ID NO: 247 - protein, 121 aa, synthetic construct
QVQLVQSGAEVKKPGASVKVSCKASGYTFTNYWIGWVRQAPGQGLEWMGDIYPGGIGGGYTKYAQKLQGRVTMTADTSTSTAYMELRSLRSDDTAVYYCSRSETGRAMDYWGQGTLVTVSS

SEQ ID NO: 248 - protein, 121 aa, synthetic construct
QVQLVQSGAEVKKPGASVKVSCKASGYTFTNYWIGWVRQAPGQGLEWMGDIYPGGIGGGYTKYAQKFQGRVTMTADTSTSTAYMELSSLRSEDTAVYYCSRSETGRAMDYWGQGTLVTVSS

SEQ ID NO: 249 - protein, 121 aa, synthetic construct
QVQLVQSGAEVKKPGASVKVSCKASGYTFTNYWIGWVRQAPGQGLEWMGDIYPGGIGGGYTKYNEKFKGRVTMTADTSTSTAYMELSSLRSEDTAVYFCSRSETGRAMDYWGQGTLVTVSS

SEQ ID NO: 250 - protein, 121 aa, synthetic construct
QVQLVQSGAELKRPGASVKVSCKASGYTFTNYWMGWVKQAPGQGLEWMGDIYPGGIGGGYTNYAQKFKGKATMTADTSSSTAYMQLSRLRSEDTAVYYCSRSETGRAMDYWGQGTLVTVSS

SEQ ID NO: 251 - protein, 106 aa, synthetic construct
DIQMTQSPSSLSASVGDRVTITCQASQDINKYLAWYQQKPGKAPKLLIHYTSTLKPGVPSRFSGSGSGTDFTFTISSLQPEDIATYYCLQYDNLNTFGGGTKLEIK

SEQ ID NO: 252 - protein, 106 aa, synthetic construct
DIQMTQSPSSLSASVGDRVTITCKASQDINKYIAWYQQKPGKAPKLLIHYTSTLKPGVPSRFSGSGSGRDYTFTISSLQPEDIATYYCLQYDNLNTFGGGTKLEIK

SEQ ID NO: 253 - protein, 106 aa, synthetic construct
DIQMTQSPSSLSASVGDRVTITCQASQDINKYLAWYQQKPGKAPKLLIYYTSTLETGVPSRFSGSGSGTDFTFSISSLQPEDIATYYCLQYDNLNTFGGGTKLEIK

SEQ ID NO: 254 - protein, 118 aa, synthetic construct
QVQLVQSGAEVKKPGASVKVSCKASGYTFADYYINWVRQAPGKGLEWMGWIFPGSGSTYYNEKFKGRVTMTVDKSTSTAYMELSSLRSEDTAVYFCARGDSGRAMDYWGQGTLVTVSS

SEQ ID NO: 255 - protein, 118 aa, synthetic construct
QVQLVQSGAEVKKPGASVKVSCKASGYTFADYYMNWVRQAPGQGLEWMGWIFPGSGSTYYAEKFKGRVTSTRDTSISTAYMELSRLRSDDTVVYYCARGDSGRAMDYWGQGTLVTVSS

SEQ ID NO: 256 - protein, 118 aa, synthetic construct
QVQLVQSGAEVKKPGASVKVSCKASGYTFADYYINWVRQAPGQGLEWMGWIFPGSGSTYYAQKLQGRVTMTTDTSTSTAYMELRSLRSDDTAVYYCARGDSGRAMDYWGQGTLVTVSS

SEQ ID NO: 257 - protein, 118 aa, synthetic construct
QVQLVQSGAEVKKPGASVKVSCKASGYTFADYYINWVRQAPGQGLEWMGWIFPGSGSTYYAQKLQGRVTMTVDKSSSTAYMELRSLRSDDTAVYYCARGDSGRAMDYWGQGTLVTVSS

SEQ ID NO: 258 - protein, 118 aa, synthetic construct
QVQLVQSGAELKKPGASVKVSCKASGYTFADYYMNWVRQAPGQGLEWMGWIFPGSGSTYYNQKFQGRVTMTVDKSSSTAYMELSRLRSDDTAVYYCARGDSGRAMDYWGQGTLVTVSS

SEQ ID NO: 259 - protein, 106 aa, synthetic construct
DIQMTQSPSSLSASVGDRVTITCKASQDINKYIAWYQQKPGKAPKLLIHYTSTLQSGVPSRFSGSGSGTDFTFTISSLQPEDIATYYCLQYDNLLTFGQGTKLEIK

SEQ ID NO: 260 - protein, 106 aa, synthetic construct
DIQMTQSPSSLSASVGDRVTITCKASQDINKYIAWYQHKPGKAPKLLIHYTSTLQSGVPSRFSGSGSGRDYTFTISSLQPEDIATYYCLQYDNLLTFGQGTKLEIK

SEQ ID NO: 261 - protein, 106 aa, synthetic construct
DIQMTQSPSSLSASVGDRVTITCQASQDINKYLAWYQHKPGKAPKLLIHYTSTLETGVPSRFSGSGSGTDFTFTISSLQPEDIATYYCLQYDNLLTFGGGTKVEIK

SEQ ID NO: 262 - protein, 118 aa, synthetic construct
QVQLQESGPGLVKPSETLSLTCTVSGFSLTIYDVHWIRQPPGKGLEWIGVIWSDGSTDYNPSLKSRVTISKDTSKNQVSLKLSSVTAADTAVYYCARNWVDQAWFAYWGQGTLVTVSS

SEQ ID NO: 263 - protein, 118 aa, synthetic construct
QVQLQESGPGLVKPSETLSLTCTVSGGSITIYDWHWVRQPPGKGLEWIGVIWSDGSTDYNPSLKSRVTISKDTSKNQFSLKLSSVTAADTAVYYCARNWVDQAWFAYWGQGTLVTVSS

SEQ ID NO: 264 - protein, 118 aa, synthetic construct
QVQLQESGPGLVKPSETLSLTCTVSGGSITIYDWHWVRQPPGKGLEWIGVIWSDGSTDYNPSLKSRVTISVDTSKNQFSLKLSSVTAADTAVYYCARNWVDQAWFAYWGQGTLVTVSS

SEQ ID NO: 265 - protein, 118 aa, synthetic construct
QVQLQESGPGLVKPSETLSLTCTVSGGSITIYDWHWIRQPPGKGLEWIGVIWSDGSTDYNPSLKSRVTISVDTSKNQFSLKLSSVTAADTAVYYCARNWVDQAWFAYWGQGTLVTVSS

SEQ ID NO: 266 - protein, 7 aa, synthetic construct
DYSFTGY

SEQ ID NO: 267 - protein, 6 aa, synthetic construct
HPYYGG

SEQ ID NO: 268 - protein, 10 aa, synthetic construct
ERSNFHALDY

SEQ ID NO: 269 - protein, 5 aa, synthetic construct
GYNMN

SEQ ID NO: 270 - protein, 17 aa, synthetic construct
NIHPYYGGTSFNQKFMG

SEQ ID NO: 271 - protein, 119 aa, synthetic construct
EIQLQQSGAELVKPGASVKISCKASDYSFTGYNMNWVMQSHGKSLEWIGNIHPYYGGTSFNQKFMGKATLTADKSSSTAYMQLNSLTSEDSAVYYCARERSNFHALDYWGQGTSVTVSS

SEQ ID NO: 272 - protein, 111 aa, synthetic construct; identical to SEQ ID NO: 153

SEQ ID NO: 273 - protein, 16 aa, synthetic construct
VIWSDGSTDYNPSLKS

SEQ ID NO: 274 - protein, 11 aa, synthetic construct
QASQDINKYLA

SEQ ID NO: 275 - protein, 7 aa, synthetic construct
YTSTLET

SEQ ID NO: 276 - protein, 5 aa, synthetic construct
DYYMN

SEQ ID NO: 277 - protein, 17 aa, synthetic construct
WIFPGSGSTYYNQKFQG

SEQ ID NO: 278 - protein, 17 aa, synthetic construct
WIFPGSGSTYYAQKLQG

SEQ ID NO: 279 - skipped

SEQ ID NO: 280 - protein, 15 aa, synthetic construct
RASESVDNDGIRFLH

SEQ ID NO: 281 - protein, 7 aa, synthetic construct
RASNRET

SEQ ID NO: 282 - protein, 17 aa, synthetic construct
WIYPLRGSINYAQKFQG

SEQ ID NO: 283 - protein, 120 aa, synthetic construct
QVQLVQSGAEVKKPGASVKVSCKASGYTFTDYTIHWVRQATGQGLEWMGWIYPLRGSINYAQKFQGRVTMTANKSISTVYMELSSLRSEDTAVYFCARHGAYYSNAFDYWGQGTLVTVSS

SEQ ID NO: 284 - protein, 111 aa, synthetic construct
EIVMTQSPATLSVSPGERATLSCRASESVDNDGIRFLHWYQQKPGQAPRLLIYRASNRETGIPARFSGSGSGTEFTLTISSLQSEDFAVYYCQQSNKDPYTFGGGTKVEIK

SEQ ID NO: 285 - protein, 7 aa, synthetic construct
RASTRAT

SEQ ID NO: 286 - protein, 111 aa, synthetic construct
EIVMTQSPATLSVSPGERATLSCRASESVDNDGIRFLHWYQQKPGQAPRLLIYRASTRATGIPARFSGSGSRTEFTLTISSLQSEDFAVYYCQQSNKDPYTFGGGTKVEIK

SEQ ID NO: 287 - protein, 17 aa, synthetic construct
WIYPLRGSINYSPSFQG

SEQ ID NO: 288 - protein, 120 aa, synthetic construct
EVQLVQSGAEVKKPGESLKISCKASGYTFTDYTIHWVRQMPGKGLEWMGWIYPLRGSINYSPSFQGQVTISADKSISTVYLQWSSLKASDTAMYFCARHGAYYSNAFDYWGQGTLVTVSS

SEQ ID NO: 289 - protein, 120 aa, synthetic construct
QVQLVQSGAEVKKPGSSVKVSCKASGYTFTDYTIHWVRQAPGQGLEWMGWIYPLRGSINYAQKFQGRVTITADKSTSTAYMELSSLRSEDTAVYFCARHGAYYSNAFDYWGQGTLVTVSS

SEQ ID NO: 290 - protein, 17 aa, synthetic construct
WIYPLRGSINYAEKFKG

SEQ ID NO: 291 - protein, 120 aa, synthetic construct
QVQLVQSGAEVKKPGSSVKVSCKASGYTFTDYTIHWVRQAPGQGLEWMGWIYPLRGSINYAEKFKGRVTLTADKSTSTVYMELSSLRSEDTAVYFCARHGAYYSNAFDYWGQGTLVTVSS

SEQ ID NO: 292 - protein, 120 aa, synthetic construct
QVQLVQSGAEVKKPGASVKVSCKASGYTFTDYTIHWVRQAPGQGLEWMGWIYPLRGSINYAQKFQGRVTMTADKSISTVYMELSRLRSDDTAVYYCARHGAYYSNAFDYWGQGTLVTVSS

SEQ ID NO: 293 - protein, 7 aa, synthetic construct
RASTLET

SEQ ID NO: 294 - protein, 120 aa, synthetic construct
QVQLVQSGAEVKKPGASVKVSCKASGYTFTDYTIHWVRQATGQGLEWMGWIYPLRGSINYAQKFQGRVTMTANKSSSTVYMELSSLRSEDTAVYFCARHGAYYSNAFDYWGQGTLVTVSS

SEQ ID NO: 295 - protein, 111 aa, synthetic construct
EIVMTQSPATLSVSPGERATLSCRASESVDNDGIRFLHWYQQKPGQAPRLLIYRASTLETGIPARFSGSGSGTEFTLTISSLQSEDFAVYYCQQSNKDPYTFGGGTKVEIK

SEQ ID NO: 296 - protein, 120 aa, synthetic construct
QVQLVQSGAEVKKPGASVKVSCKASGYTFTDYTIHWVRQATGQGLEWMGWIYPLRGSINYAQKFQGRVTMTADKSISTVYMELSSLRSEDTAVYFCARHGAYYSNAFDYWGQGTLVTVSS

SEQ ID NO: 297 - DNA, 357 bp, synthetic construct
  source 1..357: mol_type = other DNA
gagatccagctgcagcagtctggagctgaactggtgaagcctggggcttcagtgaagatatcctgcaaggcttctgattactcattcactggctacaacatgaactgggtgatgcagagccatggaaagagccttgagtggattggaaatattcatccttactatggtggtactagcttcaatcagaagttcatgggcaaggccacattgactgcagacaaatcttccagcacagcctacatgcagctcaacagcctgacatctgaagactctgcagtctattactgtgcaagagagagaagtaacttccatgctctggactactggggtcagggaacctcagtcaccgtctcctca

SEQ ID NO: 298 - DNA, 333 bp, synthetic construct; identical to SEQ ID NO: 185
  source 1..333: mol_type = other DNA

SEQ ID NO: 299 - DNA, 354 bp, synthetic construct
  source 1..354: mol_type = other DNA
caggtacaactccaggaatccgggcctgggctcgtcaaaccaagcgaaacactctctctcacctgcaccgtttctgggttttctcttactatctatgacgtacattgggtaaggcaaccacccgggaaggggctggagtggatcggtgtaatctggtcagatggatctacagactacaacccatcccttaaaagcagggtgaccatttctaaggacacttccaagaaccaagtatcccttaaattgtcctctgtaaccgcagcagacaccgcagtttactactgcgcacgaaattgggttgaccaagcatggtttgcatattggggacagggaactcttgtcactgtgtcttca

SEQ ID NO: 300 - DNA, 321 bp, synthetic construct
  source 1..321: mol_type = other DNA
gatattcaaatgacccaatccccctcatcactttcagcatctgtcggtgatcgggtcaccattacttgcagagccagtaagaatatctacagctacctggcttggtatcagcaaaaacctggtaaggcccctaaacttctcgtttacaatgctaagacccttcccgagggagttccttccaggttttccggtagcgggagtggaacagatttcaccttgactatttctagcttgcagcccgaggatttcgctacatactactgccagcatcactatggaacccccctgaccttcggtcagggaaccaagctcgagatcaaa

SEQ ID NO: 301 - DNA, 354 bp, synthetic construct
  source 1..354: mol_type = other DNA
caagtccagctcgtacagagcggggcagagctgaagaagcctggggcctccgtcaaggtctcctgtaaggcttctggttacacatttgccgactactacatgaactgggtacggcaagccccaggtcaagggctggaatggatgggatggatttttccagggagcggcagcacttactacaaccagaaatttcaaggtcgtgtgacaatgaccgtggataaaagcagctctacagcttacatggagctttcccgcttgaggtccgatgatactgccgtatattattgtgcccgtggtgactcaggtagggccatggactattggggacagggcaccctcgtgaccgtgtccagc

SEQ ID NO: 302 - DNA, 318 bp, synthetic construct
  source 1..318: mol_type = other DNA
gatatccagatgacacaatccccttcatccttgagcgcatcagttggcgacagggtcaccataacttgtcaggctagtcaggatattaacaagtacctggcttggtatcaacacaagcctggaaaggcccccaaattgctgattcactacacctctacattggaaactggcgtacccagtcgcttttctgggagtggaagcggaactgatttcactttcactatatccagtcttcagccagaagatatcgcaacttactattgtcttcagtatgataacttgcttactttcggaggagggaccaaagttgaaatcaag

SEQ ID NO: 303 - DNA, 354 bp, synthetic construct
  source 1..354: mol_type = other DNA
caggtgcagttggtccaatccggggctgaggtgaagaagcctggggcctctgttaaagttagttgcaaggcatcaggctacaccttcgctgactactacatcaactgggttagacaggcccccgggcaggggttggagtggatgggttggatttttccaggatcaggttcaacatattacgcacaaaaactgcaaggtagagtaaccatgacaactgatactagcacctccacagcctatatggaactccgctctctcaggagtgacgatacagccgtttattactgcgcccgtggggattcaggccgtgcaatggattactggggtcaagggaccctcgtgaccgtaagttca

SEQ ID NO: 304 - DNA, 360 bp, synthetic construct
  source 1..360: mol_type = other DNA
caagttcagttggtgcaaagcggggcagaagtgaagaaacctggtgcttctgtgaaagtttcctgcaaggccagcggctacacctttactgattacacaatacactgggtacggcaggcaactgggcaaggattggaatggatggggtggatatacccattgcgagggtctataaactacgcacagaaatttcaaggtcgagtaacaatgacagccaacaaatcaataagcaccgtttatatggaactctcatctctcaggagtgaggataccgccgtgtatttctgcgcacgacacggtgcatattactcaaacgctttcgactattggggccagggcacccttgtgactgttagtagc

SEQ ID NO: 305 - DNA, 333 bp, synthetic construct
  source 1..333: mol_type = other DNA
gagatagtaatgactcagtctcccgctacacttagtgtaagcccaggggagcgagcaaccctcagttgcagagcatctgagagtgttgataatgatggaatacgttttctccattggtatcaacaaaaaccagggcaggcccccagattgctgatctaccgtgcttccaatcgcgagactggcattcctgcacgtttcagcggcagcggctccggaaccgagtttacacttactattagctcactccagtctgaagacttcgctgtgtattactgtcagcaatccaacaaggacccatacactttcggaggcggcactaaggttgagatcaaa

SEQ ID NO: 306 - DNA, 333 bp, synthetic construct
  source 1..333: mol_type = other DNA
gagatagttatgactcagtctcccgccacactttcagtaagtcccggtgaacgcgccaccctgtcctgccgtgcttccgaatcagtggataatgacggcattaggtttttgcactggtaccaacaaaagcccggacaggccccccgcctgctgatatatcgtgcatcaacacgagcaacagggatccccgctcgatttagtggatccggaagcaggaccgaatttacacttaccatttcctcacttcagtcagaagatttcgccgtttactactgtcagcagtcaaataaggatccttacacatttgggggcggtacaaaagtcgagatcaaa

SEQ ID NO: 307 - DNA, 360 bp, synthetic construct
  source 1..360: mol_type = other DNA
gaggtccagttggtccagtcaggagccgaagtcaagaagcctggggaaagcctgaaaataagttgcaaagctagtggatatacatttacagattataccattcattgggtccggcaaatgccaggaaaaggcttggagtggatggggtggatttatcccctccgaggctcaataaattatagtcctagttttcaggggcaggtaactattagcgctgataaaagtatttctacagtttatttgcagtggagttcattgaaggctagtgacaccgctatgtatttctgcgctagacatggtgcatattattcaaatgccttcgactattggggccagggcaccctcgtcactgtgagttcc

SEQ ID NO: 308 - DNA, 360 bp, synthetic construct
  source 1..360: mol_type = other DNA
caggtgcaacttgttcagtcaggggctgaagtaaagaagccaggctcatcagtcaaggtatcatgcaaagcatctggctatacatttacagattacaccattcactgggtgaggcaagctcccggtcaaggtctcgagtggatggggtggatataccctctcagaggctctataaattacgctcagaaatttcaagggagagttacaattactgctgataaaagtaccagcactgcttatatggagctttcctcacttcgttcagaggacaccgccgtttacttttgtgcccggcatggtgcctattattcaaatgccttcgattattgggggcagggaactttggtcacagtttcatct

SEQ ID NO: 309 - DNA, 360 bp, synthetic construct
  source 1..360: mol_type = other DNA
caagttcaacttgtccaaagtggggctgaagttaaaaaacctggatcatcagtcaaggtttcatgcaaagccagcggttacacatttacagactatacaatacattgggttcgacaggctcccgggcaagggctcgaatggatgggatggatttatcccctcaggggctcaattaactatgctgagaaatttaagggtcgtgtaacactcaccgccgataaatccacctcaaccgtatatatggagctttcttctcttcgctctgaagataccgccgtctatttctgcgcacgacacggggcatactattctaatgcttttgactactggggacaagggacacttgtgaccgttagtagc

SEQ ID NO: 310 - DNA, 360 bp, synthetic construct
  source 1..360: mol_type = other DNA
caagtgcagttggtccagagtggagcagaggtgaagaagcctggtgcttccgtcaaggtgagttgcaaggcatctggttatactttcactgactacacaattcattgggtcaggcaggcccctggacagggactggaatggatgggatggatctatccacttagaggatcaatcaactatgctcaaaagttccagggtcgtgtaacaatgaccgcagacaaaagtatctcaactgtatacatggaattgtcccgattgaggagcgacgacacagccgtatattattgtgccaggcacggagcctactacagtaatgccttcgactactgggggcagggcacccttgttaccgtgtccagc

SEQ ID NO: 311 - DNA, 360 bp, synthetic construct
  source 1..360: mol_type = other DNA
caagtgcagctcgttcagtctggcgcagaagtgaagaagccaggagcttccgttaaagtgtcctgtaaagcctctggatatacattcacagattatacaattcactgggtgagacaagcaaccggtcaaggtctcgaatggatgggctggatataccccctccgaggttccatcaactacgctcaaaaattccaaggacgagtcactatgacagcaaacaagagttcctccactgtatatatggaactctctagtttgcgctctgaagacaccgccgtgtacttctgtgccaggcacggcgcatactattctaatgcatttgactattgggggcagggcacattggtaacagttagttcc

SEQ ID NO: 312 - DNA, 333 bp, synthetic construct
  source 1..333: mol_type = other DNA
gaaattgtaatgacccagagccccgccacccttagtgtgtccccaggcgagagggccactctttcttgccgcgcaagcgaatccgtagacaacgatggtataagatttttgcattggtatcagcaaaagccaggccaggcaccccggcttctcatctacagagctagcaccctcgaaactggaatccccgctcgtttttcaggatctggtagcggaacagaatttactttgacaattagtagtttgcagtcagaggactttgctgtctattattgccagcagtctaataaagatccatacaccttcggcggagggaccaaagtagagattaaa

SEQ ID NO: 313 - DNA, 360 bp, synthetic construct
  source 1..360: mol_type = other DNA
caagttcagttggtgcaaagcggggcagaagtgaagaaacctggtgcttctgtgaaagtttcctgcaaggccagcggctacacctttactgattacacaatacactgggtacggcaggcaactgggcaaggattggaatggatggggtggatatacccattgcgagggtctataaactacgcacagaaatttcaaggtcgagtaacaatgacagccgacaaatcaataagcaccgtttatatggaactctcatctctcaggagtgaggataccgccgtgtatttctgcgcacgacacggtgcatattactcaaacgctttcgactattggggccagggcacccttgtgactgttagtagc

SEQ ID NO: 314 - protein, 15 aa, synthetic construct
KSSQSVDNDGIRFLH

SEQ ID NO: 315 - protein, 7 aa, synthetic construct
RASTRES

SEQ ID NO: 316 - protein, 111 aa, synthetic construct
DIVMTQSPDSLAVSLGERATINCKSSQSVDNDGIRFLHWYQQKPGQPPKLLIYRASTRESGVPDRFSGSGSGTDFTLTISSLQAEDVAVYYCQQSNKDPYTFGGGTKVEIK

SEQ ID NO: 317 - protein, 120 aa, synthetic construct
QVQLVQSGAEVKKPGASVKVSCKASGYTFTDYTIHWVRQAPGQGLEWMGWIYPLRGSINYAQKFQGRVTLTADKSTSTVYMELSSLRSEDTAVYFCARHGAYYSNAFDYWGQGTLVTVSS

SEQ ID NO: 318 - DNA, 333 bp, synthetic construct
  source 1..333: mol_type = other DNA
gacattgtaatgacccagtctcccgatagcctcgctgtctcactcggagaacgcgcaaccatcaactgcaagtcctcccaaagcgttgacaatgacggcattaggtttttgcactggtaccagcagaaacccggtcaacctcctaagttgctcatttaccgagcatctacccgcgagtcaggagtacctgatcgcttttccggtagcggtagtggaacagattttactctgaccattagttcactccaggcagaagatgtggctgtctactactgccaacagtcaaataaagacccttataccttcggtgggggtaccaaagtagagatcaaa

SEQ ID NO: 319 - DNA, 360 bp, synthetic construct
  source 1..360: mol_type = other DNA
caggtgcagttggtccagagcggggcagaggttaagaagcctggggcctcagtaaaggtatcctgcaaggcttctgggtacaccttcacagattacactattcattgggtgcgccaagcacctggtcaaggccttgaatggatgggatggatttaccccttgcgagggagtattaattatgcacagaagttccagggaagggttactcttaccgccgacaagtccacatcaaccgtttacatggagctttcctctctcaggtccgaagacactgctgtatatttctgcgctcggcatggggcttattacagcaacgccttcgattactggggtcagggtacattggtcacagtgtccagt

SEQ ID NO: 320 - protein, 327 aa, synthetic construct
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 321 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 322 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSP

SEQ ID NO: 323 - protein, 323 aa, synthetic construct
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSP

SEQ ID NO: 324 - protein, 323 aa, synthetic construct
STKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSP

SEQ ID NO: 325 - protein, 323 aa, synthetic construct
STKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSP

SEQ ID NO: 326 - protein, 323 aa, synthetic construct
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSP

SEQ ID NO: 327 - protein, 15 aa, synthetic construct
  VARIANT 1: note = R or K
  VARIANT 2: note = A or S
  VARIANT 4: note = E or Q
  VARIANT 14: note = M or L
XXSXSVDNDGIRFXH

SEQ ID NO: 328 - skipped

SEQ ID NO: 329 - protein, 17 aa, synthetic construct
  VARIANT 12: note = N, S, or A
  VARIANT 13: note = E, P, or Q
  VARIANT 14: note = K or S
  VARIANT 16: note = K or Q
  VARIANT 17: note = D or G
WIYPLRGSINYXXXFXX

SEQ ID NO: 330 - protein, 8 aa, synthetic construct
  VARIANT 3: note = S or T
  VARIANT 4: note = I or F
  VARIANT 6: note = S or absent
  VARIANT 7: note = G, D or S
GYXXTXXY

SEQ ID NO: 331-332 - skipped (2 entries)

SEQ ID NO: 333 - protein, 15 aa, synthetic construct
  VARIANT 1: note = R or K
  VARIANT 4: note = E or Q
  VARIANT 7: note = D or S
  VARIANT 8: note = N, F, I or N
  VARIANT 9: note = Y, A, I or D
  VARIANT 11: note = I or T
  VARIANT 12: note = S, N or R
  VARIANT 13: note = F, L or S
  VARIANT 14: note = M or I
  VARIANT 15: note = N or H
XASXSVXXXGXXXXX

SEQ ID NO: 334 - skipped

SEQ ID NO: 335 - protein, 9 aa, synthetic construct
  VARIANT 1: note = Q or L
  VARIANT 4: note = K, R or N
  VARIANT 5: note = E or K
  VARIANT 6: note = V, Y, I or D
  VARIANT 8: note = R, W or Y
XQSXXXPXT

SEQ ID NO: 336 - skipped

SEQ ID NO: 337 - protein, 17 aa, synthetic construct
  VARIANT 1: note = Y, R or W
  VARIANT 3: note = N, Y or absent
  VARIANT 4: note = S or P
  VARIANT 5: note = Y, L or R
  VARIANT 6: note = D, N or R
  VARIANT 7: note = G or S
  VARIANT 8: note = Y, D or S
  VARIANT 9: note = N, T or I
  VARIANT 10: note = N, F or K
  VARIANT 13: note = P, Q or E
  VARIANT 14: note = S or K
  VARIANT 15: note = L or F
  VARIANT 17: note = N, G or D
XIXXXXXXXXYNXXXKX

SEQ ID NO: 338 - protein, 4 aa, synthetic construct
DDSD

SEQ ID NO: 339 - protein, 8 aa, synthetic construct
VTFTMGQV

SEQ ID NO: 340 - protein, 4 aa, synthetic construct
MPSH

SEQ ID NO: 341 - protein, 4 aa, synthetic construct
PRAR

SEQ ID NO: 342 - protein, 16 aa, synthetic construct
  VARIANT 12: note = A or P
  VARIANT 13: note = A or S
  VARIANT 14: note = F or L
  VARIANT 15: note = I or K
VIWSDGSTDYNXXXXS

SEQ ID NO: 343 - protein, 5 aa, synthetic construct
  VARIANT 4: note = I or M
DYYXN

SEQ ID NO: 344 - protein, 17 aa, synthetic construct
  VARIANT 12: note = N or A
  VARIANT 13: note = E or Q
  VARIANT 15: note = F or L
  VARIANT 16: note = K or Q
WIFPGSGSTYYXXKXXG

SEQ ID NO: 345 - protein, 11 aa, synthetic construct
  VARIANT 1: note = K or Q
XASQDINKYIA

SEQ ID NO: 346 - protein, 7 aa, synthetic construct
  VARIANT 6: note = Q or E
  VARIANT 7: note = S or T
YTSTLXX

SEQ ID NO: 347 - protein, 17 aa, Homo sapiens
KQHSVLHLVPINATSKD

(19) United States
(12) Patent Application Publication
(10) Pub. No.: US 2024/0092921 A1
(43) Pub. Date: Mar. 21, 2024

(54) ANTIBODY MOLECULES TO APRIL AND USES THEREOF

(71) Applicant: VISTERRA, INC., Waltham, MA (US)

(72) Inventors: David William Oldach, Jamaica Plain, MA (US); James R. Myette, Waltham, MA (US); Zachary Shriver, Winchester, MA (US); Karthik Viswanathan, Acton, MA (US); Andrew M. Wollacott, Milton, MA (US); Hedy Adari-Hall, Sudbury, MA (US); Boopathy Ramakrishnan, Braintree, MA (US); Gregory Babcock, Marlborough, MA (US); Jill Yarbrough, Pittsboro, NC (US); Asher Schachter, Needham, MA (US); Mohit Mathur, Lexington, MA (US)

(21) Appl. No.: 18/305,667

(22) Filed: Apr. 24, 2023

Related U.S. Application Data
(60) Provisional application No. 63/374,646, filed on Sep. 6, 2022.
(60) Provisional application No. 63/334,381, filed on Apr. 25, 2022.

(51) Int. Cl. C07K 16/28; A61K 38/17; A61K 39/395; A61P 13/12

(52) U.S. Cl. CPC C07K 16/2875; A61K 38/1793; A61K 39/3955; A61P 13/12; C07K 16/2887; A61K 2039/505; C07K 2317/24

(57) ABSTRACT

Antibody molecules that specifically bind to APRIL are disclosed. The antibody molecules can be used to treat, prevent, and/or diagnose disorders, such as IgA nephropathy.

### CROSS-REFERENCE TO RELATED APPLICATIONS

This application claims the benefit of U.S. Provisional Application No. 63/374,646, filed Sep. 6, 2022, and U.S. Provisional Application No. 63/334,381, filed Apr. 25, 2022. The contents of the aforementioned applications are hereby incorporated by reference in their entirety.

### SEQUENCE LISTING

The instant application contains a Sequence Listing which has been submitted electronically in XML format and is hereby incorporated by reference in its entirety. Said XML copy, created on Apr. 20, 2023, is named P2029-704710_SL.xml and is 348,201 bytes in size.

### BACKGROUND

IgA nephropathy is one of the most prevalent, chronic glomerular diseases worldwide. Conservative epidemiological estimates cite a global incidence of approximately 5-50 cases/million (children) and 10-40 cases/million (adults). This incidence of disease presents a regional bias with a higher prevalence in Asia and the Americas, with a particularly higher disease burden in Japan and regions of China. Biopsy confirmed cases of IgA nephropathy in Japan are projected at approximately 350,000. In the US, this projection is approximately 100,000-as such, it is the most frequently diagnosed 1° glomerular disease in adults. While a relatively indolent disease, IgA nephropathy leads to end stage renal disease (ESRD), i.e., renal failure in 20-50% of patients within a 20-30 year span. These numbers are likely grossly underreported given the need to confirm the disease by kidney biopsy, a protocol that is variably practiced in various clinical settings. The disease has a complex pathogenesis with genetic, epidemiological, and potentially environmental components to disease etiology, pathology, and progression. It likewise has a variable clinical presentation ranging from asymptomatic to end-stage renal failure (ESRD). IgA nephropathy is caused by the deposition of IgA, typically in the form of immune complexes in the mesangium of the kidney. There are currently no disease-specific treatments to address primary disease or progression.

There is a need for developing new approaches for treating, preventing and diagnosing IgA nephropathy and other disorders that share similar disease mechanisms.

### SUMMARY

Accordingly, in certain aspects, this disclosure provides a method of improving kidney function, the method comprising administering to a subject in need thereof an anti-APRIL antibody molecule (e.g., an anti-APRIL antibody molecule as described herein), thereby improving kidney function. In some embodiments, the method reverses or prevents progression of decreased kidney function in the subject. In some embodiments, the improved kidney function comprises kidney regeneration. In some embodiments, the improved kidney function comprises increased estimated glomerular filtrate rate (eGFR) in the kidney of the subject. In some embodiments, the improved kidney function comprises reduced proteinuria in the kidney of the subject.

In an aspect, the disclosure provides a method of preserving (e.g., maintaining or increasing) eGFR in the kidney of a subject in need thereof, the method comprising administering to the subject an anti-APRIL antibody molecule (e.g., an anti-APRIL antibody molecule as described herein), thereby preserving (e.g., maintaining or increasing) eGFR in the kidney of the subject.

In some embodiments, the average eGFR over a period of at least twelve months after administration of the antibody molecule is greater than or equal to the subject's baseline eGFR. In some embodiments, the administration maintains eGFR in the kidney of the subject, e.g., the average eGFR over a period of at least twelve months after administration of the antibody molecule is equal to, or substantially equal to, the subject's baseline eGFR. In some embodiments, the administration increases eGFR in the kidney of the subject, e.g., the average eGFR over a period of at least twelve months after administration of the antibody molecule is greater than the subject's baseline eGFR. In some embodiments, the subject's baseline eGFR is the eGFR prior to administration of the antibody molecule.

In an aspect, the disclosure provides a method of reducing proteinuria in the kidney of a subject in need thereof, the method comprising administering to the subject an anti-APRIL antibody molecule (e.g., an anti-APRIL antibody molecule as described herein), thereby reducing proteinuria in the kidney of the subject. In some embodiments, the reduction in proteinuria is determined by measuring urine protein/creatinine ratio (uPCR), e.g., as described herein.

In an aspect, the disclosure provides a method of inducing kidney recovery in a subject in need thereof, the method comprising administering to the subject an anti-APRIL antibody molecule (e.g., an anti-APRIL antibody molecule as described herein), thereby inducing kidney recovery in the kidney of the subject.

In an aspect, the disclosure provides a method of inducing kidney regeneration in a subject in need thereof, the method comprising administering to the subject an anti-APRIL antibody molecule (e.g., an anti-APRIL antibody molecule as described herein), thereby inducing kidney regeneration in the kidney of the subject.

In an aspect, the disclosure provides a method of reducing autoantibody response in a subject in need thereof, the method comprising administering to the subject an anti-APRIL antibody molecule (e.g., an anti-APRIL antibody molecule as described herein), thereby reducing autoantibody response in the subject.

In some embodiments of any of the aspects disclosed herein, the anti-APRIL antibody molecule comprises a heavy chain variable region (VH) comprising three heavy chain complementarity determining regions (HCDR1, HCDR2, and HCDR3) and/or a light chain variable region (VL) comprising three light chain complementarity determining regions (LCDR1, LCDR2, and LCDR3), wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and/or the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and/or the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16.

In an aspect, the disclosure also provides a method for treating a disorder, the method comprising administering to a subject in need thereof an anti-APRIL antibody molecule described herein, wherein the antibody molecule is administered at a dose that reduces, or is likely to reduce, the level of aberrantly glycosylated IgA (a-g IgA), e.g. aberrantly glycosylated IgA1 (a-g IgA1), by at least 40% in the subject, thereby treating the disorder. In an embodiment, the level of a-g IgA comprises, or is, the level of a-g IgA1.

In an embodiment, the level of a-g IgA is reduced by at least 40% about 4 weeks after the antibody molecule is administered. In an embodiment, the level of a-g IgA is reduced by at least 40% about 8 weeks after the antibody molecule is administered. In an embodiment, the level of a-g IgA is reduced by at least 40% about 12 weeks after the antibody molecule is administered. In an embodiment, the level of a-g IgA is reduced by at least 40% about 16 weeks after the antibody molecule is administered. In an embodiment, the level of a-g IgA is reduced by at least 40% for a predetermined period, e.g., at least one, two, three, or four weeks, or at least one, two, or three months. In an embodiment, the level of a-g IgA is reduced by at least 50%. In an embodiment, the level of a-g IgA is reduced by at least 55%, 60%, 65%, 70%, 75%, 80%, 85%, 90%, or 95%. In an embodiment, the antibody molecule is administered as a single dose. In an embodiment, the antibody molecule is administered as a repeated dose. In an embodiment, the antibody molecule is administered subcutaneously. In an embodiment, the antibody molecule is administered intravenously.

In an embodiment, the disorder is an APRIL-associated disorder. In an embodiment, the disorder is associated with an aberrant level of total IgA. In an embodiment, the disorder is a disorder associated with a-g IgA (e.g., a-g IgA1).

In an embodiment, the disorder is IgA nephropathy (IgAN). In an embodiment, the IgAN is a familial IgAN. In an embodiment, the IgA is an adult IgAN. In an embodiment, the IgAN is a post-transplant IgAN, a pediatric IgAN, or a crescentic IgAN.

In an embodiment, the disorder is a chronic kidney disease (CKD) or a disorder associated with CKD. In an embodiment, the CKD is an advanced CKD, e.g., with an estimated glomerular filtration rate (eGFR) equal to or greater than about 30 or about 45.

In an embodiment, the disorder is Henoch-Schonlein purpura (HSP). In an embodiment, the disorder is cutaneous vasculitis or IgA vasculitis. In an embodiment, the disorder is IgA dermatitis, e.g., IgA bullous dermatosis. In an embodiment, the disorder is Waldenstrom macroglobulinemia (WM). In an embodiment, the disorder is lupus nephritis.

In an embodiment, the subject is a human. In an embodiment, the subject has, or is identified as having, a level of a-g IgA that is at least 1, 1.5, 2, 2.5, 3.5, 4, 4.5, or 5-fold higher than the level of a-g IgA in a reference subject, e.g., a subject who does not have the disorder, e.g., a healthy or normal subject. In an embodiment, the subject has, or is identified as having, a level of total IgA that is at least 1, 1.5, 2, 2.5, 3.5, 4, 4.5, or 5-fold higher than the level of total IgA in a reference subject, e.g., a subject who does not have the disorder, e.g., a healthy or normal subject. In an embodiment, the subject has received, or is receiving, a different therapeutic agent or modality for treating the disorder. In an embodiment, the subject has not received, or is not receiving, a different therapeutic agent or modality for treating the disorder. In an embodiment, the subject has, or is identified as having, a genomic susceptible locus of the disorder, e.g., IgA nephropathy. In an embodiment, the method further comprises determining whether the subject has a genomic susceptible locus of the disorder, e.g., IgA nephropathy.

In an embodiment, the anti-APRIL antibody molecule is an anti-APRIL antibody molecule described herein. In an embodiment, the anti-APRIL antibody molecule comprises the HCDR1, HCDR2, HCDR3, LCDR1, LCDR2, and LCDR3 of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237. In an embodiment, the anti-APRIL antibody molecule comprises the VH and VL of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237.

In an embodiment, the level of a-g IgA is determined in a sample from the subject. In an embodiment, the method described herein further comprises determining the level of a-g IgA in a sample from the subject. In an embodiment, the method further comprises determining the level of total IgA in the sample. In an embodiment, the method further comprises determining the level of IgM and/or IgG in the sample. In an embodiment, the method further comprises obtaining a sample from the subject. In an embodiment, the sample is a blood or serum sample.

In an embodiment, the method further comprises administering a second therapeutic agent or modality to the subject. In an embodiment, the second therapeutic agent or modality is a small molecule.

In an embodiment, the second therapeutic agent or modality is an antibody molecule.

In an embodiment, the subject has received, is receiving, or is going to receive, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject is, or is identified as being, in need of receiving, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject receives the vaccine before, concurrent with, or after administration of the anti-APRIL antibody molecule.

In an embodiment, administration of the anti-APRIL antibody molecule reduces the subject's ability to have an effective antigen-specific serum IgG and/or IgA response to the vaccine by no more than 5%, 10%, 15%, 20%, 25%, 30%, 35%, 40%, 45%, or 50%. In an embodiment, administration of the anti-APRIL antibody molecule does not reduce, or does not substantially reduce, the subject's ability to have an effective antigen-specific serum IgG and/or IgA response to the vaccine. In an embodiment, the subject has or maintains an effective (e.g., protective) antigen-specific serum IgG and/or IgA response to the vaccine after administration of the anti-APRIL antibody molecule.

In an embodiment, the vaccine comprises tetanus toxoid, diphtheria toxoid, or both (e.g., TENIVAC®). In an embodiment, the subject has or maintains an effective (e.g., protective) level of tetanus and/or diphtheria anti-toxoid IgG (e.g., equal to or above 0.1 IU/mL in the blood), e.g., 1, 2, 3, 4, 5, 6, 7, 8, 9, 10, 11, 12, 13, 14, 15, 16, or more weeks after administration of the anti-APRIL antibody molecule.

In an aspect, the disclosure features a method of treating a disorder, the method comprising administering to a subject in need thereof an anti-APRIL antibody molecule, wherein the administration reduces the level of a-g IgA (e.g., a-g IgA1) by at least 40% in the subject, thereby treating the disorder.

In an embodiment, the level of a-g IgA comprises, or is, the level of a-g IgA1.

In an embodiment, the level of a-g IgA is reduced by at least 40% about 4 weeks after the antibody molecule is administered. In an embodiment, the level of a-g IgA is reduced by at least 40% about 8 weeks after the antibody molecule is administered. In an embodiment, the level of a-g IgA is reduced by at least 40% about 12 weeks after the antibody molecule is administered. In an embodiment, the level of a-g IgA is reduced by at least 40% about 16 weeks after the antibody molecule is administered. In an embodiment, the level of a-g IgA is reduced by at least 40% for a predetermined period, e.g., at least one, two, three, or four weeks, or at least one, two, or three months. In an embodiment, the level of a-g IgA is reduced by at least 50%. In an embodiment, the level of a-g IgA is reduced by at least 55%, 60%, 65%, 70%, 75%, 80%, 85%, 90%, or 95%. In an embodiment, the antibody molecule is administered as a single dose. In an embodiment, the antibody molecule is administered as a repeated dose. In an embodiment, the antibody molecule is administered subcutaneously. In an embodiment, the antibody molecule is administered intravenously.

In an embodiment, the disorder is an APRIL-associated disorder. In an embodiment, the disorder is associated with an aberrant level of total IgA. In an embodiment, the disorder is a disorder associated with a-g IgA (e.g., a-g IgA1).

In an embodiment, the disorder is IgA nephropathy (IgAN). In an embodiment, the IgAN is a familial IgAN. In an embodiment, the IgA is an adult IgAN. In an embodiment, the IgAN is a post-transplant IgAN, a pediatric IgAN, or a crescentic IgAN.

In an embodiment, the disorder is a chronic kidney disease (CKD) or a disorder associated with CKD. In an embodiment, the CKD is an advanced CKD, e.g., with an estimated glomerular filtration rate (eGFR) equal to or greater than about 30 or about 45.

In an embodiment, the disorder is Henoch-Schonlein purpura (HSP). In an embodiment, the disorder is cutaneous vasculitis or IgA vasculitis. In an embodiment, the disorder is IgA dermatitis, e.g., IgA bullous dermatosis. In an embodiment, the disorder is Waldenstrom macroglobulinemia (WM). In an embodiment, the disorder is lupus nephritis.

In an embodiment, the subject is a human. In an embodiment, the subject has, or is identified as having, a level of a-g IgA that is at least 1, 1.5, 2, 2.5, 3.5, 4, 4.5, or 5-fold higher than the level of a-g IgA in a reference subject, e.g., a subject who does not have the disorder, e.g., a healthy or normal subject. In an embodiment, the subject has, or is identified as having, a level of total IgA that is at least 1, 1.5, 2, 2.5, 3.5, 4, 4.5, or 5-fold higher than the level of total IgA in a reference subject, e.g., a subject who does not have the disorder, e.g., a healthy or normal subject. In an embodiment, the subject has received, or is receiving, a different therapeutic agent or modality for treating the disorder. In an embodiment, the subject has not received, or is not receiving, a different therapeutic agent or modality for treating the disorder. In an embodiment, the subject has, or is identified as having, a genomic susceptible locus of the disorder, e.g., IgA nephropathy. In an embodiment, the method described herein further comprises determining whether the subject has a genomic susceptible locus of the disorder, e.g., IgA nephropathy.

In an embodiment, the anti-APRIL antibody molecule is an anti-APRIL antibody molecule described herein. In an embodiment, the anti-APRIL antibody molecule comprises the HCDR1, HCDR2, HCDR3, LCDR1, LCDR2, and LCDR3 of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237. In an embodiment, the anti-APRIL antibody molecule comprises the VH and VL of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237.

In an embodiment, the level of a-g IgA is determined in a sample from the subject. In an embodiment, the method further comprises determining the level of a-g IgA in a sample from the subject.

In an embodiment, the method further comprises determining the level of total IgA in the sample. In an embodiment, the method further comprising determining the level of IgM and/or IgG in the sample. In an embodiment, the method further comprises obtaining a sample from the subject. In an embodiment, the sample is a blood or serum sample.

In an embodiment, the method further comprises administering a second therapeutic agent or modality to the subject. In an embodiment, the second therapeutic agent or modality is a small molecule.

In an embodiment, the second therapeutic agent or modality is an antibody molecule.

In an embodiment, the subject has received, is receiving, or is going to receive, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject is, or is identified as being, in need of receiving, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject receives the vaccine before, concurrent with, or after administration of the anti-APRIL antibody molecule.

In an embodiment, administration of the anti-APRIL antibody molecule reduces the subject's ability to have an effective antigen-specific serum IgG and/or IgA response to the vaccine by no more than 5%, 10%, 15%, 20%, 25%, 30%, 35%, 40%, 45%, or 50%. In an embodiment, administration of the anti-APRIL antibody molecule does not reduce, or does not substantially reduce, the subject's ability to have an effective antigen-specific serum IgG and/or IgA response to the vaccine. In an embodiment, the subject has or maintains an effective (e.g., protective) antigen-specific serum IgG and/or IgA response to the vaccine after administration of the anti-APRIL antibody molecule.

In an embodiment, the vaccine comprises tetanus toxoid, diphtheria toxoid, or both (e.g., TENIVAC®). In an embodiment, the subject has or maintains an effective (e.g., protective) level of tetanus and/or diphtheria anti-toxoid IgG (e.g., equal to or above 0.1 IU/mL in the blood), e.g., 1, 2, 3, 4, 5, 6, 7, 8, 9, 10, 11, 12, 13, 14, 15, 16, or more weeks after administration of the anti-APRIL antibody molecule.

In yet another aspect, the disclosure features a method of treating a disorder, the method comprising administering to a subject in need thereof an anti-APRIL antibody molecule, wherein the antibody molecule is administered at a dosage (e.g., dose and frequency) that reduces, or is likely to reduce, the level of a-g IgA (e.g., a-g IgA1) by at least 40% in the subject, thereby treating the disorder.

In an embodiment, the level of a-g IgA comprises, or is, the level of a-g IgA1.

In an embodiment, the level of a-g IgA is reduced by at least 40% about 4 weeks after the antibody molecule is administered. In an embodiment, the level of a-g IgA is reduced by at least 40% about 8 weeks after the antibody molecule is administered. In an embodiment, the level of a-g IgA is reduced by at least 40% about 12 weeks after the antibody molecule is administered. In an embodiment, the level of a-g IgA is reduced by at least 40% about 16 weeks after the antibody molecule is administered. In an embodiment, the level of a-g IgA is reduced by at least 40% for a predetermined period, e.g., at least one, two, three, or four weeks, or at least one, two, or three months. In an embodiment, the level of a-g IgA is reduced by at least 50%. In an embodiment, the level of a-g IgA is reduced by at least 55%, 60%, 65%, 70%, 75%, 80%, 85%, 90%, or 95%. In an embodiment, the antibody molecule is administered as a single dose. In an embodiment, the antibody molecule is administered as a repeated dose. In an embodiment, the antibody molecule is administered subcutaneously. In an embodiment, the antibody molecule is administered intravenously.

In an embodiment, the disorder is an APRIL-associated disorder. In an embodiment, the disorder is associated with an aberrant level of total IgA. In an embodiment, the disorder is a disorder associated with a-g IgA (e.g., a-g IgA1).

In an embodiment, the disorder is IgA nephropathy (IgAN). In an embodiment, the IgAN is a familial IgAN. In an embodiment, the IgA is an adult IgAN. In an embodiment, the IgAN is a post-transplant IgAN, a pediatric IgAN, or a crescentic IgAN.

In an embodiment, the disorder is a chronic kidney disease (CKD) or a disorder associated with CKD. In an embodiment, the CKD is an advanced CKD, e.g., with an estimated glomerular filtration rate (eGFR) equal to or greater than about 30 or about 45.

In an embodiment, the disorder is Henoch-Schonlein purpura (HSP). In an embodiment, the disorder is cutaneous vasculitis or IgA vasculitis. In an embodiment, the disorder is IgA dermatitis, e.g., IgA bullous dermatosis. In an embodiment, the disorder is Waldenstrom macroglobulinemia (WM). In an embodiment, the disorder is lupus nephritis.

In an embodiment, the subject is a human. In an embodiment, the subject has, or is identified as having, a level of a-g IgA that is at least 1, 1.5, 2, 2.5, 3.5, 4, 4.5, or 5-fold higher than the level of a-g IgA in a reference subject, e.g., a subject who does not have the disorder, e.g., a healthy or normal subject. In an embodiment, the subject has, or is identified as having, a level of total IgA that is at least 1, 1.5, 2, 2.5, 3.5, 4, 4.5, or 5-fold higher than the level of total IgA in a reference subject, e.g., a subject who does not have the disorder, e.g., a healthy or normal subject. In an embodiment, the subject has received, or is receiving, a different therapeutic agent or modality for treating the disorder. In an embodiment, the subject has not received, or is not receiving, a different therapeutic agent or modality for treating the disorder. In an embodiment, the subject has, or is identified as having, a genomic susceptible locus of the disorder, e.g., IgA nephropathy. In an embodiment, the method further comprises determining whether the subject has a genomic susceptible locus of the disorder, e.g., IgA nephropathy.

In an embodiment, the anti-APRIL antibody molecule is an anti-APRIL antibody molecule described herein. In an embodiment, the anti-APRIL antibody molecule comprises the HCDR1, HCDR2, HCDR3, LCDR1, LCDR2, and LCDR3 of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237. In an embodiment, the anti-APRIL antibody molecule comprises the VH and VL of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237.

In an embodiment, the level of a-g IgA is determined in a sample from the subject. In an embodiment, the method further comprises determining the level of a-g IgA in a sample from the subject.

In an embodiment, the method further comprises determining the level of total IgA in the sample. In an embodiment, the method further comprising determining the level of IgM and/or IgG in the sample. In an embodiment, the method further comprises obtaining a sample from the subject. In an embodiment, the sample is a blood or serum sample.

In an embodiment, the method further comprises administering a second therapeutic agent or modality to the subject. In an embodiment, the second therapeutic agent or modality is a small molecule.

In an embodiment, the second therapeutic agent or modality is an antibody molecule.

In an embodiment, the subject has received, is receiving, or is going to receive, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject is, or is identified as being, in need of receiving, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject receives the vaccine before, concurrent with, or after administration of the anti-APRIL antibody molecule.

In an embodiment, administration of the anti-APRIL antibody molecule reduces the subject's ability to have an effective antigen-specific serum IgG and/or IgA response to the vaccine by no more than 5%, 10%, 15%, 20%, 25%, 30%, 35%, 40%, 45%, or 50%. In an embodiment, administration of the anti-APRIL antibody molecule does not reduce, or does not substantially reduce, the subject's ability to have an effective antigen-specific serum IgG and/or IgA response to the vaccine. In an embodiment, the subject has or maintains an effective (e.g., protective) antigen-specific serum IgG and/or IgA response to the vaccine after administration of the anti-APRIL antibody molecule.

In an embodiment, the vaccine comprises tetanus toxoid, diphtheria toxoid, or both (e.g., TENIVAC®). In an embodiment, the subject has or maintains an effective (e.g., protective) level of tetanus and/or diphtheria anti-toxoid IgG (e.g., equal to or above 0.1 IU/mL in the blood), e.g., 1, 2, 3, 4, 5, 6, 7, 8, 9, 10, 11, 12, 13, 14, 15, 16, or more weeks after administration of the anti-APRIL antibody molecule.

In another aspect, the disclosure features a method of treating a disorder, the method comprising selecting a dose or dosage (e.g., dose and frequency) for an anti-APRIL antibody molecule, wherein administration of the antibody molecule at the dose or dosage reduces, or is likely to reduce, the level of a-g IgA (e.g., a-g IgA1) by at least 40% in a subject in need thereof, and administering the antibody molecule to the subject at the selected dose or dosage, thereby treating the disorder. In an embodiment, the level of a-g IgA comprises, or is, the level of a-g IgA1.

In an embodiment, the anti-APRIL antibody molecule reduces, or is likely to reduce, the level of a-g IgA by at least 40% about 4 weeks after the antibody molecule is administered. In an embodiment, the anti-APRIL antibody molecule reduces, or is likely to reduce, the level of a-g IgA by at least 40% about 8 weeks after the antibody molecule is administered. In an embodiment, the anti-APRIL antibody molecule reduces, or is likely to reduce, the level of a-g IgA by at least 40% about 12 weeks after the antibody molecule is administered. In an embodiment, the anti-APRIL antibody molecule reduces, or is likely to reduce, the level of a-g IgA by at least 40% about 16 weeks after the antibody molecule is administered. In an embodiment, the anti-APRIL antibody molecule reduces, or is likely to reduce, the level of a-g IgA by at least 40% for a predetermined period, e.g., at least one, two, three, or four weeks, or at least one, two, or three months. In an embodiment, the anti-APRIL antibody molecule reduces, or is likely to reduce, the level of a-g IgA by at least 50%. In an embodiment, the anti-APRIL antibody molecule reduces, or is likely to reduce, the level of a-g IgA by at least 55%, 60%, 65%, 70%, 75%, 80%, 85%, 90%, or 95%. In an embodiment, the antibody molecule is administered as a single dose. In an embodiment, the antibody molecule is administered as a repeated dose. In an embodiment, the antibody molecule is administered subcutaneously. In an embodiment, the antibody molecule is administered intravenously.

In an embodiment, the disorder is an APRIL-associated disorder. In an embodiment, the disorder is associated with an aberrant level of total IgA. In an embodiment, the disorder is a disorder associated with a-g IgA (e.g., a-g IgA1).

In an embodiment, the disorder is IgA nephropathy (IgAN). In an embodiment, the IgAN is a familial IgAN. In an embodiment, the IgA is an adult IgAN. In an embodiment, the IgAN is a post-transplant IgAN, a pediatric IgAN, or a crescentic IgAN.

In an embodiment, the disorder is a chronic kidney disease (CKD) or a disorder associated with CKD. In an embodiment, the CKD is an advanced CKD, e.g., with an estimated glomerular filtration rate (eGFR) equal to or greater than about 30 or about 45.

In an embodiment, the disorder is Henoch-Schonlein purpura (HSP). In an embodiment, the disorder is cutaneous vasculitis or IgA vasculitis. In an embodiment, the disorder is IgA dermatitis, e.g., IgA bullous dermatosis. In an embodiment, the disorder is Waldenstrom macroglobulinemia (WM). In an embodiment, the disorder is lupus nephritis.

In an embodiment, the subject is a human. In an embodiment, the subject has, or is identified as having, a level of a-g IgA that is at least 1, 1.5, 2, 2.5, 3.5, 4, 4.5, or 5-fold higher than the level of a-g IgA in a reference subject, e.g., a subject who does not have the disorder, e.g., a healthy or normal subject. In an embodiment, the subject has, or is identified as having, a level of total IgA that is at least 1, 1.5, 2, 2.5, 3.5, 4, 4.5, or 5-fold higher than the level of total IgA in a reference subject, e.g., a subject who does not have the disorder, e.g., a healthy or normal subject. In an embodiment, the subject has received, or is receiving, a different therapeutic agent or modality for treating the disorder. In an embodiment, the subject has not received, or is not receiving, a different therapeutic agent or modality for treating the disorder. In an embodiment, the subject has, or is identified as having, a genomic susceptible locus of the disorder, e.g., IgA nephropathy. In an embodiment, the method further comprises determining whether the subject has a genomic susceptible locus of the disorder, e.g., IgA nephropathy.

In an embodiment, the anti-APRIL antibody molecule is an anti-APRIL antibody molecule described herein. In an embodiment, the anti-APRIL antibody molecule comprises the HCDR1, HCDR2, HCDR3, LCDR1, LCDR2, and LCDR3 of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237. In an embodiment, the anti-APRIL antibody molecule comprises the VH and VL of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237.

In an embodiment, the level of a-g IgA is determined in a sample from the subject. In an embodiment, the method further comprises determining the level of a-g IgA in a sample from the subject.

In an embodiment, the method further comprises determining the level of total IgA in the sample. In an embodiment, the method further comprising determining the level of IgM and/or IgG in the sample. In an embodiment, the method further comprises obtaining a sample from the subject. In an embodiment, the sample is a blood or serum sample.

In an embodiment, the method further comprises administering a second therapeutic agent or modality to the subject. In an embodiment, the second therapeutic agent or modality is a small molecule.

In an embodiment, the second therapeutic agent or modality is an antibody molecule.

In an embodiment, the subject has received, is receiving, or is going to receive, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject is, or is identified as being, in need of receiving, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject receives the vaccine before, concurrent with, or after administration of the anti-APRIL antibody molecule.

In an embodiment, administration of the anti-APRIL antibody molecule reduces the subject's ability to have an effective antigen-specific serum IgG and/or IgA response to the vaccine by no more than 5%, 10%, 15%, 20%, 25%, 30%, 35%, 40%, 45%, or 50%. In an embodiment, administration of the anti-APRIL antibody molecule does not reduce, or does not substantially reduce, the subject's ability to have an effective antigen-specific serum IgG and/or IgA response to the vaccine. In an embodiment, the subject has or maintains an effective (e.g., protective) antigen-specific serum IgG and/or IgA response to the vaccine after administration of the anti-APRIL antibody molecule.

In an embodiment, the vaccine comprises tetanus toxoid, diphtheria toxoid, or both (e.g., TENIVAC®). In an embodiment, the subject has or maintains an effective (e.g., protective) level of tetanus and/or diphtheria anti-toxoid IgG (e.g., equal to or above 0.1 IU/mL in the blood), e.g., 1, 2, 3, 4, 5, 6, 7, 8, 9, 10, 11, 12, 13, 14, 15, 16, or more weeks after administration of the anti-APRIL antibody molecule.

In an aspect, the disclosure features a method of treating a disorder, the method comprising responsive to a determination that administration of an anti-APRIL antibody molecule reduces, or is likely to reduce, the level of a-g IgA (e.g., a-g IgA1) by at least 40% in a subject in need thereof, administering to the subject an anti-APRIL antibody molecule, thereby treating the disorder. In an embodiment, the level of a-g IgA comprises, or is, the level of a-g IgA1.

In an embodiment, the anti-APRIL antibody molecule reduces, or is likely to reduce, the level of a-g IgA by at least 40% about 4 weeks after the antibody molecule is administered. In an embodiment, the anti-APRIL antibody molecule reduces, or is likely to reduce, the level of a-g IgA by at least 40% about 8 weeks after the antibody molecule is administered. In an embodiment, the anti-APRIL antibody molecule reduces, or is likely to reduce, the level of a-g IgA by at least 40% about 12 weeks after the antibody molecule is administered. In an embodiment, the anti-APRIL antibody molecule reduces, or is likely to reduce, the level of a-g IgA by at least 40% about 16 weeks after the antibody molecule is administered. In an embodiment, the anti-APRIL antibody molecule reduces, or is likely to reduce, the level of a-g IgA by at least 40% for a predetermined period, e.g., at least one, two, three, or four weeks, or at least one, two, or three months. In an embodiment, the anti-APRIL antibody molecule reduces, or is likely to reduce, the level of a-g IgA by at least 50%. In an embodiment, the anti-APRIL antibody molecule reduces, or is likely to reduce, the level of a-g IgA by at least 55%, 60%, 65%, 70%, 75%, 80%, 85%, 90%, or 95%. In an embodiment, the antibody molecule is administered as a single dose. In an embodiment, the antibody molecule is administered as a repeated dose. In an embodiment, the antibody molecule is administered subcutaneously. In an embodiment, the antibody molecule is administered intravenously.

In an embodiment, the disorder is an APRIL-associated disorder. In an embodiment, the disorder is associated with an aberrant level of total IgA. In an embodiment, the disorder is a disorder associated with a-g IgA (e.g., a-g IgA1).

In an embodiment, the disorder is IgA nephropathy (IgAN). In an embodiment, the IgAN is a familial IgAN. In an embodiment, the IgA is an adult IgAN. In an embodiment, the IgAN is a post-transplant IgAN, a pediatric IgAN, or a crescentic IgAN.

In an embodiment, the disorder is a chronic kidney disease (CKD) or a disorder associated with CKD. In an embodiment, the CKD is an advanced CKD, e.g., with an estimated glomerular filtration rate (eGFR) equal to or greater than about 30 or about 45.

In an embodiment, the disorder is Henoch-Schonlein purpura (HSP). In an embodiment, the disorder is cutaneous vasculitis or IgA vasculitis. In an embodiment, the disorder is IgA dermatitis, e.g., IgA bullous dermatosis. In an embodiment, the disorder is Waldenstrom macroglobulinemia (WM). In an embodiment, the disorder is lupus nephritis.

In an embodiment, the subject is a human. In an embodiment, the subject has, or is identified as having, a level of a-g IgA that is at least 1, 1.5, 2, 2.5, 3.5, 4, 4.5, or 5-fold higher than the level of a-g IgA in a reference subject, e.g., a subject who does not have the disorder, e.g., a healthy or normal subject. In an embodiment, the subject has, or is identified as having, a level of total IgA that is at least 1, 1.5, 2, 2.5, 3.5, 4, 4.5, or 5-fold higher than the level of total IgA in a reference subject, e.g., a subject who does not have the disorder, e.g., a healthy or normal subject. In an embodiment, the subject has received, or is receiving, a different therapeutic agent or modality for treating the disorder. In an embodiment, the subject has not received, or is not receiving, a different therapeutic agent or modality for treating the disorder. In an embodiment, the subject has, or is identified as having, a genomic susceptible locus of the disorder, e.g., IgA nephropathy. In an embodiment, the method further comprises determining whether the subject has a genomic susceptible locus of the disorder, e.g., IgA nephropathy.

In an embodiment, the anti-APRIL antibody molecule is an anti-APRIL antibody molecule described herein. In an embodiment, the anti-APRIL antibody molecule comprises the HCDR1, HCDR2, HCDR3, LCDR1, LCDR2, and LCDR3 of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237. In an embodiment, the anti-APRIL antibody molecule comprises the VH and VL of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237.

In an embodiment, the level of a-g IgA is determined in a sample from the subject. In an embodiment, the method further comprises determining the level of a-g IgA in a sample from the subject.

In an embodiment, the method further comprises determining the level of total IgA in the sample. In an embodiment, the method further comprising determining the level of IgM and/or IgG in the sample. In an embodiment, the method further comprises obtaining a sample from the subject. In an embodiment, the sample is a blood or serum sample.

In an embodiment, the method further comprises administering a second therapeutic agent or modality to the subject. In an embodiment, the second therapeutic agent or modality is a small molecule.

In an embodiment, the second therapeutic agent or modality is an antibody molecule.

In an embodiment, the subject has received, is receiving, or is going to receive, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject is, or is identified as being, in need of receiving, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject receives the vaccine before, concurrent with, or after administration of the anti-APRIL antibody molecule.

In an embodiment, administration of the anti-APRIL antibody molecule reduces the subject's ability to have an effective antigen-specific serum IgG and/or IgA response to the vaccine by no more than 5%, 10%, 15%, 20%, 25%, 30%, 35%, 40%, 45%, or 50%. In an embodiment, administration of the anti-APRIL antibody molecule does not reduce, or does not substantially reduce, the subject's ability to have an effective antigen-specific serum IgG and/or IgA response to the vaccine. In an embodiment, the subject has or maintains an effective (e.g., protective) antigen-specific serum IgG and/or IgA response to the vaccine after administration of the anti-APRIL antibody molecule.

In an embodiment, the vaccine comprises tetanus toxoid, diphtheria toxoid, or both (e.g., TENIVAC®). In an embodiment, the subject has or maintains an effective (e.g., protective) level of tetanus and/or diphtheria anti-toxoid IgG (e.g., equal to or above 0.1 IU/mL in the blood), e.g., 1, 2, 3, 4, 5, 6, 7, 8, 9, 10, 11, 12, 13, 14, 15, 16, or more weeks after administration of the anti-APRIL antibody molecule.

In another aspect, the disclosure features a method of treating a disorder, the method comprising determining whether administration of an anti-APRIL antibody molecule reduces, or is likely to reduce, the level of a-g IgA (e.g., a-g IgA1) by at least 40% in a subject in need thereof, if the antibody molecule reduces, or is likely to reduce, the level of a-g IgA by at least 40%, administration of the antibody molecule is initiated, continued, or maintained. In an embodiment, the level of a-g IgA comprises, or is, the level of a-g IgA1.

In an embodiment, if the antibody molecule does not reduce, or is not likely to reduce, the level of a-g IgA by at least 40%, administration of the antibody molecule is terminated, discontinued, or altered. In an embodiment, if the antibody molecule does not reduce, or is not likely to reduce, the level of a-g IgA by at least 40%, a different therapeutic agent or modality is administered.

In an embodiment, the level of a-g IgA is reduced by at least 40% about 4 weeks after the antibody molecule is administered. In an embodiment, the level of a-g IgA is reduced by at least 40% about 8 weeks after the antibody molecule is administered. In an embodiment, the level of a-g IgA is reduced by at least 40% about 12 weeks after the antibody molecule is administered. In an embodiment, the level of a-g IgA is reduced by at least 40% about 16 weeks after the antibody molecule is administered. In an embodiment, the level of a-g IgA is reduced by at least 40% for a predetermined period, e.g., at least one, two, three, or four weeks, or at least one, two, or three months. In an embodiment, the level of a-g IgA is reduced by at least 50%. In an embodiment, the level of a-g IgA is reduced by at least 55%, 60%, 65%, 70%, 75%, 80%, 85%, 90%, or 95%. In an embodiment, the antibody molecule is administered as a single dose. In an embodiment, the antibody molecule is administered as a repeated dose. In an embodiment, the antibody molecule is administered subcutaneously. In an embodiment, the antibody molecule is administered intravenously.

In an embodiment, the disorder is an APRIL-associated disorder. In an embodiment, the disorder is associated with an aberrant level of total IgA. In an embodiment, the disorder is a disorder associated with a-g IgA (e.g., a-g IgA1).

In an embodiment, the disorder is IgA nephropathy (IgAN). In an embodiment, the IgAN is a familial IgAN. In an embodiment, the IgA is an adult IgAN. In an embodiment, the IgAN is a post-transplant IgAN, a pediatric IgAN, or a crescentic IgAN.

In an embodiment, the disorder is a chronic kidney disease (CKD) or a disorder associated with CKD. In an embodiment, the CKD is an advanced CKD, e.g., with an estimated glomerular filtration rate (eGFR) equal to or greater than about 30 or about 45.

In an embodiment, the disorder is Henoch-Schonlein purpura (HSP). In an embodiment, the disorder is cutaneous vasculitis or IgA vasculitis. In an embodiment, the disorder is IgA dermatitis, e.g., IgA bullous dermatosis. In an embodiment, the disorder is Waldenstrom macroglobulinemia (WM). In an embodiment, the disorder is lupus nephritis.

In an embodiment, the subject is a human. In an embodiment, the subject has, or is identified as having, a level of a-g IgA that is at least 1, 1.5, 2, 2.5, 3.5, 4, 4.5, or 5-fold higher than the level of a-g IgA in a reference subject, e.g., a subject who does not have the disorder, e.g., a healthy or normal subject. In an embodiment, the subject has, or is identified as having, a level of total IgA that is at least 1, 1.5, 2, 2.5, 3.5, 4, 4.5, or 5-fold higher than the level of total IgA in a reference subject, e.g., a subject who does not have the disorder, e.g., a healthy or normal subject. In an embodiment, the subject has received, or is receiving, a different therapeutic agent or modality for treating the disorder. In an embodiment, the subject has not received, or is not receiving, a different therapeutic agent or modality for treating the disorder. In an embodiment, the subject has, or is identified as having, a genomic susceptible locus of the disorder, e.g., IgA nephropathy. In an embodiment, the method further comprises determining whether the subject has a genomic susceptible locus of the disorder, e.g., IgA nephropathy.

In an embodiment, the anti-APRIL antibody molecule is an anti-APRIL antibody molecule described herein. In an embodiment, the anti-APRIL antibody molecule comprises the HCDR1, HCDR2, HCDR3, LCDR1, LCDR2, and LCDR3 of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237. In an embodiment, the anti-APRIL antibody molecule comprises the VH and VL of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237.

In an embodiment, the level of a-g IgA is determined in a sample from the subject. In an embodiment, the method further comprises determining the level of a-g IgA in a sample from the subject.

In an embodiment, the method further comprises determining the level of total IgA in the sample. In an embodiment, the method further comprising determining the level of IgM and/or IgG in the sample. In an embodiment, the method further comprises obtaining a sample from the subject. In an embodiment, the sample is a blood or serum sample.

In an embodiment, the method further comprises administering a second therapeutic agent or modality to the subject. In an embodiment, the second therapeutic agent or modality is a small molecule.

In an embodiment, the second therapeutic agent or modality is an antibody molecule.

In an embodiment, the subject has received, is receiving, or is going to receive, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject is, or is identified as being, in need of receiving, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject receives the vaccine before, concurrent with, or after administration of the anti-APRIL antibody molecule.

In an embodiment, administration of the anti-APRIL antibody molecule reduces the subject's ability to have an effective antigen-specific serum IgG and/or IgA response to the vaccine by no more than 5%, 10%, 15%, 20%, 25%, 30%, 35%, 40%, 45%, or 50%. In an embodiment, administration of the anti-APRIL antibody molecule does not reduce, or does not substantially reduce, the subject's ability to have an effective antigen-specific serum IgG and/or IgA response to the vaccine. In an embodiment, the subject has or maintains an effective (e.g., protective) antigen-specific serum IgG and/or IgA response to the vaccine after administration of the anti-APRIL antibody molecule.

In an embodiment, the vaccine comprises tetanus toxoid, diphtheria toxoid, or both (e.g., TENIVAC®). In an embodiment, the subject has or maintains an effective (e.g., protective) level of tetanus and/or diphtheria anti-toxoid IgG (e.g., equal to or above 0.1 IU/mL in the blood), e.g., 1, 2, 3, 4, 5, 6, 7, 8, 9, 10, 11, 12, 13, 14, 15, 16, or more weeks after administration of the anti-APRIL antibody molecule.

In yet another aspect, the disclosure features a method of treating a disorder, the method comprising determining whether administration of an anti-APRIL antibody molecule at a dose or dosage reduces, or is likely to reduce, the level of a-g IgA1 (e.g., a-g IgA1) by at least 40% in a subject in need thereof, if the antibody molecule reduces, or is likely to reduce, the level of a-g IgA by at least 40% at the dose or dosage, administration of the antibody molecule at the dose or dosage is initiated, continued, or maintained. In an embodiment, the level of a-g IgA comprises, or is, the level of a-g IgA1.

In an embodiment, if the antibody molecule does not reduce, or is not likely to reduce, the level of a-g IgA by at least 40% at the dose or dosage, administration of the antibody molecule at the dose or dosage is terminated, discontinued, or altered.

In an embodiment, the level of a-g IgA is reduced by at least 40% about 4 weeks after the antibody molecule is administered. In an embodiment, the level of a-g IgA is reduced by at least 40% about 8 weeks after the antibody molecule is administered. In an embodiment, the level of a-g IgA is reduced by at least 40% about 12 weeks after the antibody molecule is administered. In an embodiment, the level of a-g IgA is reduced by at least 40% about 16 weeks after the antibody molecule is administered. In an embodiment, the level of a-g IgA is reduced by at least 40% for a predetermined period, e.g., at least one, two, three, or four weeks, or at least one, two, or three months. In an embodiment, the level of a-g IgA is reduced by at least 50%. In an embodiment, the level of a-g IgA is reduced by at least 55%, 60%, 65%, 70%, 75%, 80%, 85%, 90%, or 95%. In an embodiment, the antibody molecule is administered as a single dose. In an embodiment, the antibody molecule is administered as a repeated dose. In an embodiment, the antibody molecule is administered subcutaneously. In an embodiment, the antibody molecule is administered intravenously.

In an embodiment, the disorder is an APRIL-associated disorder. In an embodiment, the disorder is associated with an aberrant level of total IgA. In an embodiment, the disorder is a disorder associated with a-g IgA (e.g., a-g IgA1).

In an embodiment, the disorder is IgA nephropathy (IgAN). In an embodiment, the IgAN is a familial IgAN. In an embodiment, the IgA is an adult IgAN. In an embodiment, the IgAN is a post-transplant IgAN, a pediatric IgAN, or a crescentic IgAN.

In an embodiment, the disorder is a chronic kidney disease (CKD) or a disorder associated with CKD. In an embodiment, the CKD is an advanced CKD, e.g., with an estimated glomerular filtration rate (eGFR) equal to or greater than about 30 or about 45.

In an embodiment, the disorder is Henoch-Schonlein purpura (HSP). In an embodiment, the disorder is cutaneous vasculitis or IgA vasculitis. In an embodiment, the disorder is IgA dermatitis, e.g., IgA bullous dermatosis. In an embodiment, the disorder is Waldenstrom macroglobulinemia (WM). In an embodiment, the disorder is lupus nephritis.

In an embodiment, the subject is a human. In an embodiment, the subject has, or is identified as having, a level of a-g IgA that is at least 1, 1.5, 2, 2.5, 3.5, 4, 4.5, or 5-fold higher than the level of a-g IgA in a reference subject, e.g., a subject who does not have the disorder, e.g., a healthy or normal subject. In an embodiment, the subject has, or is identified as having, a level of total IgA that is at least 1, 1.5, 2, 2.5, 3.5, 4, 4.5, or 5-fold higher than the level of total IgA in a reference subject, e.g., a subject who does not have the disorder, e.g., a healthy or normal subject. In an embodiment, the subject has received, or is receiving, a different therapeutic agent or modality for treating the disorder. In an embodiment, the subject has not received, or is not receiving, a different therapeutic agent or modality for treating the disorder. In an embodiment, the subject has, or is identified as having, a genomic susceptible locus of the disorder, e.g., IgA nephropathy. In an embodiment, the method further comprises determining whether the subject has a genomic susceptible locus of the disorder, e.g., IgA nephropathy.

In an embodiment, the anti-APRIL antibody molecule is an anti-APRIL antibody molecule described herein. In an embodiment, the anti-APRIL antibody molecule comprises the HCDR1, HCDR2, HCDR3, LCDR1, LCDR2, and LCDR3 of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237. In an embodiment, the anti-APRIL antibody molecule comprises the VH and VL of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237.

In an embodiment, the level of a-g IgA is determined in a sample from the subject. In an embodiment, the method further comprises determining the level of a-g IgA in a sample from the subject.

In an embodiment, the method further comprises determining the level of total IgA in the sample. In an embodiment, the method further comprising determining the level of IgM and/or IgG in the sample. In an embodiment, the method further comprises obtaining a sample from the subject. In an embodiment, the sample is a blood or serum sample.

In an embodiment, the method further comprises administering a second therapeutic agent or modality to the subject. In an embodiment, the second therapeutic agent or modality is a small molecule.

In an embodiment, the second therapeutic agent or modality is an antibody molecule.

In an embodiment, the subject has received, is receiving, or is going to receive, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject is, or is identified as being, in need of receiving, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject receives the vaccine before, concurrent with, or after administration of the anti-APRIL antibody molecule.

In an embodiment, administration of the anti-APRIL antibody molecule reduces the subject's ability to have an effective antigen-specific serum IgG and/or IgA response to the vaccine by no more than 5%, 10%, 15%, 20%, 25%, 30%, 35%, 40%, 45%, or 50%. In an embodiment, administration of the anti-APRIL antibody molecule does not reduce, or does not substantially reduce, the subject's ability to have an effective antigen-specific serum IgG and/or IgA response to the vaccine. In an embodiment, the subject has or maintains an effective (e.g., protective) antigen-specific serum IgG and/or IgA response to the vaccine after administration of the anti-APRIL antibody molecule.

In an embodiment, the vaccine comprises tetanus toxoid, diphtheria toxoid, or both (e.g., TENIVAC®). In an embodiment, the subject has or maintains an effective (e.g., protective) level of tetanus and/or diphtheria anti-toxoid IgG (e.g., equal to or above 0.1 IU/mL in the blood), e.g., 1, 2, 3, 4, 5, 6, 7, 8, 9, 10, 11, 12, 13, 14, 15, 16, or more weeks after administration of the anti-APRIL antibody molecule.

In an aspect, the disclosure features a method of treating a disorder, the method comprising determining whether administration of a therapeutic agent or modality other than an anti-APRIL antibody molecule described herein reduces, or is likely to reduce, the level of a-g IgA by at least 40% in a subject in need thereof, if the therapeutic agent or modality does not reduce, or is not likely to reduce, the level of a-g IgA by at least 40%, administering an anti-APRIL antibody molecule described herein to the subject.

In an embodiment, the antibody molecule is administered at a dose or dosage that reduces, or is likely to reduce, the level of a-g IgA by at least 40% in the subject. In an embodiment, the level of a-g IgA comprises, or is, the level of a-g IgA1.

In an embodiment, the therapeutic agent or modality reduces, or is likely to reduce, the level of a-g IgA by at least 40% about 4 weeks after the antibody molecule is administered. In an embodiment, the therapeutic agent or modality reduces, or is likely to reduce, the level of a-g IgA by at least 40% about 8 weeks after the antibody molecule is administered. In an embodiment, the therapeutic agent or modality reduces, or is likely to reduce, the level of a-g IgA by at least 40% about 12 weeks after the antibody molecule is administered. In an embodiment, the therapeutic agent or modality reduces, or is likely to reduce, the level of a-g IgA by at least 40% about 16 weeks after the antibody molecule is administered.

In an embodiment, the therapeutic agent or modality reduces, or is likely to reduce, the level of a-g IgA by at least 40% for a predetermined period, e.g., at least one, two, three, or four weeks, or at least one, two, or three months. In an embodiment, the therapeutic agent or modality reduces, or is likely to reduce, the level of a-g IgA by at least 50%. In an embodiment, the therapeutic agent or modality reduces, or is likely to reduce, the level of a-g IgA by at least 55%, 60%, 65%, 70%, 75%, 80%, 85%, 90%, or 95%. In an embodiment, the therapeutic agent or modality is administered as a single dose. In an embodiment, the therapeutic agent or modality is administered as a repeated dose. In an embodiment, the antibody molecule is administered subcutaneously. In an embodiment, the antibody molecule is administered intravenously.

In an embodiment, the disorder is associated with an aberrant level of total IgA. In an embodiment, the disorder is a disorder associated with a-g IgA (e.g., a-g IgA1).

In an embodiment, the disorder is IgA nephropathy (IgAN). In an embodiment, the IgAN is a familial IgAN. In an embodiment, the IgA is an adult IgAN. In an embodiment, the IgAN is a post-transplant IgAN, a pediatric IgAN, or a crescentic IgAN.

In an embodiment, the disorder is a chronic kidney disease (CKD) or a disorder associated with CKD. In an embodiment, the CKD is an advanced CKD, e.g., with an estimated glomerular filtration rate (eGFR) equal to or greater than about 30 or about 45.

In an embodiment, the disorder is Henoch-Schonlein purpura (HSP). In an embodiment, the disorder is cutaneous vasculitis or IgA vasculitis. In an embodiment, the disorder is IgA dermatitis, e.g., IgA bullous dermatosis. In an embodiment, the disorder is Waldenstrom macroglobulinemia (WM). In an embodiment, the disorder is lupus nephritis.

In an embodiment, the subject is a human. In an embodiment, the subject has, or is identified as having, a level of a-g IgA that is at least 1, 1.5, 2, 2.5, 3.5, 4, 4.5, or 5-fold higher than the level of a-g IgA in a reference subject, e.g., a subject who does not have the disorder, e.g., a healthy or normal subject. In an embodiment, the subject has, or is identified as having, a level of total IgA that is at least 1, 1.5, 2, 2.5, 3.5, 4, 4.5, or 5-fold higher than the level of total IgA in a reference subject, e.g., a subject who does not have the disorder, e.g., a healthy or normal subject. In an embodiment, the subject has received, or is receiving, a different therapeutic agent or modality for treating the disorder. In an embodiment, the subject has not received, or is not receiving, a different therapeutic agent or modality for treating the disorder. In an embodiment, the subject has, or is identified as having, a genomic susceptible locus of the disorder, e.g., IgA nephropathy. In an embodiment, the method further comprises determining whether the subject has a genomic susceptible locus of the disorder, e.g., IgA nephropathy.

In an embodiment, the anti-APRIL antibody molecule comprises the HCDR1, HCDR2, HCDR3, LCDR1, LCDR2, and LCDR3 of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237. In an embodiment, the anti-APRIL antibody molecule comprises the VH and VL of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237.

In an embodiment, the level of a-g IgA is determined in a sample from the subject. In an embodiment, the method further comprises determining the level of a-g IgA in a sample from the subject.

In an embodiment, the method further comprises determining the level of total IgA in the sample. In an embodiment, the method further comprising determining the level of IgM and/or IgG in the sample. In an embodiment, the method further comprises obtaining a sample from the subject. In an embodiment, the sample is a blood or serum sample.

In an embodiment, the method further comprises administering a second therapeutic agent or modality to the subject. In an embodiment, the second therapeutic agent or modality is a small molecule.

In an embodiment, the second therapeutic agent or modality is an antibody molecule.

In an embodiment, the subject has received, is receiving, or is going to receive, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject is, or is identified as being, in need of receiving, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject receives the vaccine before, concurrent with, or after administration of the anti-APRIL antibody molecule.

In an embodiment, administration of the anti-APRIL antibody molecule reduces the subject's ability to have an effective antigen-specific serum IgG and/or IgA response to the vaccine by no more than 5%, 10%, 15%, 20%, 25%, 30%, 35%, 40%, 45%, or 50%. In an embodiment, administration of the anti-APRIL antibody molecule does not reduce, or does not substantially reduce, the subject's ability to have an effective antigen-specific serum IgG and/or IgA response to the vaccine. In an embodiment, the subject has or maintains an effective (e.g., protective) antigen-specific serum IgG and/or IgA response to the vaccine after administration of the anti-APRIL antibody molecule.

In an embodiment, the vaccine comprises tetanus toxoid, diphtheria toxoid, or both (e.g., TENIVAC®). In an embodiment, the subject has or maintains an effective (e.g., protective) level of tetanus and/or diphtheria anti-toxoid IgG (e.g., equal to or above 0.1 IU/mL in the blood), e.g., 1, 2, 3, 4, 5, 6, 7, 8, 9, 10, 11, 12, 13, 14, 15, 16, or more weeks after administration of the anti-APRIL antibody molecule.

In another aspect the disclosure features a method of reducing the level of a-g IgA (e.g., a-g IgA1) in subject, the method comprising administering an anti-APRIL antibody molecule to a subject in a need thereof, e.g., at a dose or dosage that reduces, or is likely to reduce, the level of a-g IgA by at least 40% in the subject, thereby reducing the level of a-g IgA. In an embodiment, the level of a-g IgA comprises, or is, the level of a-g IgA1.

In an embodiment, the level of a-g IgA is reduced by at least 40% about 4 weeks after the antibody molecule is administered. In an embodiment, the level of a-g IgA is reduced by at least 40% about 8 weeks after the antibody molecule is administered. In an embodiment, the level of a-g IgA is reduced by at least 40% about 12 weeks after the antibody molecule is administered. In an embodiment, the level of a-g IgA is reduced by at least 40% about 16 weeks after the antibody molecule is administered. In an embodiment, the level of a-g IgA is reduced by at least 40% for a predetermined period, e.g., at least one, two, three, or four weeks, or at least one, two, or three months. In an embodiment, the level of a-g IgA is reduced by at least 50%. In an embodiment, the level of a-g IgA is reduced by at least 55%, 60%, 65%, 70%, 75%, 80%, 85%, 90%, or 95%. In an embodiment, the antibody molecule is administered as a single dose. In an embodiment, the antibody molecule is administered as a repeated dose. In an embodiment, the antibody molecule is administered subcutaneously. In an embodiment, the antibody molecule is administered intravenously.

In an embodiment, the subject is a human. In an embodiment, the subject has, or is identified as having, a level of a-g IgA that is at least 1, 1.5, 2, 2.5, 3.5, 4, 4.5, or 5-fold higher than the level of a-g IgA in a reference subject, e.g., a subject who does not have the disorder, e.g., a healthy or normal subject. In an embodiment, the subject has, or is identified as having, a level of total IgA that is at least 1, 1.5, 2, 2.5, 3.5, 4, 4.5, or 5-fold higher than the level of total IgA in a reference subject, e.g., a subject who does not have the disorder, e.g., a healthy or normal subject. In an embodiment, the subject has received, or is receiving, a different therapeutic agent or modality for treating the disorder. In an embodiment, the subject has not received, or is not receiving, a different therapeutic agent or modality for treating the disorder. In an embodiment, the subject has, or is identified as having, a genomic susceptible locus of the disorder, e.g., IgA nephropathy. In an embodiment, the method further comprises determining whether the subject has a genomic susceptible locus of the disorder, e.g., IgA nephropathy.

In an embodiment, the subject has or is identified as having an APRIL-associated disorder. In an embodiment, the subject has or is identified as having a disorder associated with an aberrant level of total IgA. In an embodiment, the subject has or is identified as having a disorder associated with a-g IgA (e.g., a-g IgA1).

In an embodiment, the subject has or is identified as having an IgA nephropathy (IgAN). In an embodiment, the IgAN is a familial IgAN. In an embodiment, the IgA is an adult IgAN. In an embodiment, the IgAN is a post-transplant IgAN, a pediatric IgAN, or a crescentic IgAN.

In an embodiment, the subject has or is identified as having a chronic kidney disease (CKD) or a disorder associated with CKD. In an embodiment, the CKD is an advanced CKD, e.g., with an estimated glomerular filtration rate (eGFR) equal to or greater than about 30 or about 45.

In an embodiment, the subject has or is identified as having a Henoch-Schonlein purpura (HSP).

In an embodiment, the subject has or is identified as having a cutaneous vasculitis or IgA vasculitis. In an embodiment, the subject has or is identified as having an IgA dermatitis, e.g., IgA bullous dermatosis. In an embodiment, the subject has or is identified as having a Waldenstrom macroglobulinemia (WM). In an embodiment, the subject has or is identified as having a lupus nephritis.

In an embodiment, the anti-APRIL antibody molecule is an anti-APRIL antibody molecule described herein. In an embodiment, the anti-APRIL antibody molecule comprises the HCDR1, HCDR2, HCDR3, LCDR1, LCDR2, and LCDR3 of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237. In an embodiment, the anti-APRIL antibody molecule comprises the VH and VL of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237.

In an embodiment, the level of a-g IgA is determined in a sample from the subject. In an embodiment, the method further comprises determining the level of a-g IgA in a sample from the subject.

In an embodiment, the method further comprises determining the level of total IgA in the sample. In an embodiment, the method further comprising determining the level of IgM and/or IgG in the sample. In an embodiment, the method further comprises obtaining a sample from the subject. In an embodiment, the sample is a blood or serum sample.

In an embodiment, the method further comprises administering a second therapeutic agent or modality to the subject. In an embodiment, the second therapeutic agent or modality is a small molecule.

In an embodiment, the second therapeutic agent or modality is an antibody molecule.

In an embodiment, the subject has received, is receiving, or is going to receive, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject is, or is identified as being, in need of receiving, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject receives the vaccine before, concurrent with, or after administration of the anti-APRIL antibody molecule.

In an embodiment, administration of the anti-APRIL antibody molecule reduces the subject's ability to have an effective antigen-specific serum IgG and/or IgA response to the vaccine by no more than 5%, 10%, 15%, 20%, 25%, 30%, 35%, 40%, 45%, or 50%. In an embodiment, administration of the anti-APRIL antibody molecule does not reduce, or does not substantially reduce, the subject's ability to have an effective antigen-specific serum IgG and/or IgA response to the vaccine. In an embodiment, the subject has or maintains an effective (e.g., protective) antigen-specific serum IgG and/or IgA response to the vaccine after administration of the anti-APRIL antibody molecule.

In an embodiment, the vaccine comprises tetanus toxoid, diphtheria toxoid, or both (e.g., TENIVAC®). In an embodiment, the subject has or maintains an effective (e.g., protective) level of tetanus and/or diphtheria anti-toxoid IgG (e.g., equal to or above 0.1 IU/mL in the blood), e.g., 1, 2, 3, 4, 5, 6, 7, 8, 9, 10, 11, 12, 13, 14, 15, 16, or more weeks after administration of the anti-APRIL antibody molecule.

In yet another aspect, the disclosure features a method of selecting an anti-APRIL antibody molecule for treating a disorder, the method comprising determining whether administration of the anti-APRIL antibody molecule reduces, or is likely to reduce, the level of a-g IgA (e.g., a-g IgA1) by at least 40% in a subject in need thereof, thereby selecting the anti-APRIL antibody molecule. In an embodiment, the level of a-g IgA comprises, or is, the level of a-g IgA1.

In an embodiment, the anti-APRIL antibody molecule is an anti-APRIL antibody molecule described herein. In an embodiment, the anti-APRIL antibody molecule comprises the HCDR1, HCDR2, HCDR3, LCDR1, LCDR2, and LCDR3 of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237. In an embodiment, the anti-APRIL antibody molecule comprises the VH and VL of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237.

In an embodiment, the anti-APRIL antibody molecule reduces, or is likely to reduce, the level of a-g IgA by at least 40% about 4 weeks after the antibody molecule is administered. In an embodiment, the anti-APRIL antibody molecule reduces, or is likely to reduce, the level of a-g IgA by at least 40% about 8 weeks after the antibody molecule is administered. In an embodiment, the anti-APRIL antibody molecule reduces, or is likely to reduce, the level of a-g IgA by at least 40% about 12 weeks after the antibody molecule is administered. In an embodiment, the anti-APRIL antibody molecule reduces, or is likely to reduce, the level of a-g IgA by at least 40% about 16 weeks after the antibody molecule is administered. In an embodiment, the anti-APRIL antibody molecule reduces, or is likely to reduce, the level of a-g IgA by at least 40% for a predetermined period, e.g., at least one, two, three, or four weeks, or at least one, two, or three months. In an embodiment, the anti-APRIL antibody molecule reduces, or is likely to reduce, the level of a-g IgA by at least 50%. In an embodiment, the anti-APRIL antibody molecule reduces, or is likely to reduce, the level of a-g IgA by at least 55%, 60%, 65%, 70%, 75%, 80%, 85%, 90%, or 95%. In an embodiment, the antibody molecule is administered as a single dose. In an embodiment, the antibody molecule is administered as a repeated dose. In an embodiment, the antibody molecule is administered subcutaneously. In an embodiment, the antibody molecule is administered intravenously.

In an embodiment, the subject is a human. In an embodiment, the subject has, or is identified as having, a level of a-g IgA that is at least 1, 1.5, 2, 2.5, 3.5, 4, 4.5, or 5-fold higher than the level of a-g IgA in a reference subject, e.g., a subject who does not have the disorder, e.g., a healthy or normal subject. In an embodiment, the subject has, or is identified as having, a level of total IgA that is at least 1, 1.5, 2, 2.5, 3.5, 4, 4.5, or 5-fold higher than the level of total IgA in a reference subject, e.g., a subject who does not have the disorder, e.g., a healthy or normal subject. In an embodiment, the subject has received, or is receiving, a different therapeutic agent or modality for treating the disorder. In an embodiment, the subject has not received, or is not receiving, a different therapeutic agent or modality for treating the disorder. In an embodiment, the subject has, or is identified as having, a genomic susceptible locus of the disorder, e.g., IgA nephropathy. In an embodiment, the method further comprises determining whether the subject has a genomic susceptible locus of the disorder, e.g., IgA nephropathy.

In an embodiment, the subject has or is identified as having an APRIL-associated disorder. In an embodiment, the subject has or is identified as having a disorder associated with an aberrant level of total IgA. In an embodiment, the subject has or is identified as having a disorder associated with a-g IgA (e.g., a-g IgA1).

In an embodiment, the subject has or is identified as having an IgA nephropathy (IgAN). In an embodiment, the IgAN is a familial IgAN. In an embodiment, the IgA is an adult IgAN. In an embodiment, the IgAN is a post-transplant IgAN, a pediatric IgAN, or a crescentic IgAN.

In an embodiment, the subject has or is identified as having a chronic kidney disease (CKD) or a disorder associated with CKD. In an embodiment, the CKD is an advanced CKD, e.g., with an estimated glomerular filtration rate (eGFR) equal to or greater than about 30 or about 45.

In an embodiment, the subject has or is identified as having a Henoch-Schonlein purpura (HSP).

In an embodiment, the subject has or is identified as having a cutaneous vasculitis or IgA vasculitis. In an embodiment, the subject has or is identified as having a IgA dermatitis, e.g., IgA bullous dermatosis. In an embodiment, the subject has or is identified as having a Waldenstrom macroglobulinemia (WM). In an embodiment, the subject has or is identified as having a lupus nephritis.

In an embodiment, the level of a-g IgA is determined in a sample from the subject. In an embodiment, the method further comprises determining the level of a-g IgA in a sample from the subject.

In an embodiment, the method further comprises determining the level of total IgA in the sample. In an embodiment, the method further comprising determining the level of IgM and/or IgG in the sample. In an embodiment, the method further comprises obtaining a sample from the subject. In an embodiment, the sample is a blood or serum sample.

In an embodiment, the subject has received, is receiving, or is going to receive, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject is, or is identified as being, in need of receiving, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject receives the vaccine before, concurrent with, or after administration of the anti-APRIL antibody molecule.

In an embodiment, administration of the anti-APRIL antibody molecule reduces the subject's ability to have an effective antigen-specific serum IgG and/or IgA response to the vaccine by no more than 5%, 10%, 15%, 20%, 25%, 30%, 35%, 40%, 45%, or 50%. In an embodiment, administration of the anti-APRIL antibody molecule does not reduce, or does not substantially reduce, the subject's ability to have an effective antigen-specific serum IgG and/or IgA response to the vaccine. In an embodiment, the subject has or maintains an effective (e.g., protective) antigen-specific serum IgG and/or IgA response to the vaccine after administration of the anti-APRIL antibody molecule.

In an embodiment, the vaccine comprises tetanus toxoid, diphtheria toxoid, or both (e.g., TENIVAC®). In an embodiment, the subject has or maintains an effective (e.g., protective) level of tetanus and/or diphtheria anti-toxoid IgG (e.g., equal to or above 0.1 IU/mL in the blood), e.g., 1, 2, 3, 4, 5, 6, 7, 8, 9, 10, 11, 12, 13, 14, 15, 16, or more weeks after administration of the anti-APRIL antibody molecule.

In another aspect, the disclosure features a method of selecting a dose or dosage (e.g., dose and frequency) for an anti-APRIL antibody molecule for treating a disorder, the method comprising determining whether administration of the anti-APRIL antibody molecule at a dose or dosage reduces, or is likely to reduce, the level of a-g IgA (e.g., a-g IgA1) by at least 40% in a subject in need thereof, thereby selecting the dose or dosage. In an embodiment, the level of a-g IgA comprises, or is, the level of a-g IgA1.

In an embodiment, the anti-APRIL antibody molecule is an anti-APRIL antibody molecule described herein. In an embodiment, the anti-APRIL antibody molecule comprises the HCDR1, HCDR2, HCDR3, LCDR1, LCDR2, and LCDR3 of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237. In an embodiment, the anti-APRIL antibody molecule comprises the VH and VL of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237.

In an embodiment, the anti-APRIL antibody molecule reduces, or is likely to reduce, the level of a-g IgA by at least 40% about 4 weeks after the antibody molecule is administered. In an embodiment, the anti-APRIL antibody molecule reduces, or is likely to reduce, the level of a-g IgA by at least 40% about 8 weeks after the antibody molecule is administered. In an embodiment, the anti-APRIL antibody molecule reduces, or is likely to reduce, the level of a-g IgA by at least 40% about 12 weeks after the antibody molecule is administered. In an embodiment, the anti-APRIL antibody molecule reduces, or is likely to reduce, the level of a-g IgA by at least 40% about 16 weeks after the antibody molecule is administered. In an embodiment, the anti-APRIL antibody molecule reduces, or is likely to reduce, the level of a-g IgA by at least 40% for a predetermined period, e.g., at least one, two, three, or four weeks, or at least one, two, or three months. In an embodiment, the anti-APRIL antibody molecule reduces, or is likely to reduce, the level of a-g IgA by at least 50%. In an embodiment, the anti-APRIL antibody molecule reduces, or is likely to reduce, the level of a-g IgA by at least 55%, 60%, 65%, 70%, 75%, 80%, 85%, 90%, or 95%. In an embodiment, the antibody molecule is administered as a single dose. In an embodiment, the antibody molecule is administered as a repeated dose. In an embodiment, the antibody molecule is administered subcutaneously. In an embodiment, the antibody molecule is administered intravenously.

In an embodiment, the subject is a human. In an embodiment, the subject has, or is identified as having, a level of a-g IgA that is at least 1, 1.5, 2, 2.5, 3.5, 4, 4.5, or 5-fold higher than the level of a-g IgA in a reference subject, e.g., a subject who does not have the disorder, e.g., a healthy or normal subject. In an embodiment, the subject has, or is identified as having, a level of total IgA that is at least 1, 1.5, 2, 2.5, 3.5, 4, 4.5, or 5-fold higher than the level of total IgA in a reference subject, e.g., a subject who does not have the disorder, e.g., a healthy or normal subject. In an embodiment, the subject has received, or is receiving, a different therapeutic agent or modality for treating the disorder. In an embodiment, the subject has not received, or is not receiving, a different therapeutic agent or modality for treating the disorder. In an embodiment, the subject has, or is identified as having, a genomic susceptible locus of the disorder, e.g., IgA nephropathy. In an embodiment, the method further comprises determining whether the subject has a genomic susceptible locus of the disorder, e.g., IgA nephropathy.

In an embodiment, the subject has or is identified as having an APRIL-associated disorder. In an embodiment, the subject has or is identified as having a disorder associated with an aberrant level of total IgA. In an embodiment, the subject has or is identified as having a disorder associated with a-g IgA (e.g., a-g IgA1).

In an embodiment, the subject has or is identified as having an IgA nephropathy (IgAN). In an embodiment, the IgAN is a familial IgAN. In an embodiment, the IgA is an adult IgAN. In an embodiment, the IgAN is a post-transplant IgAN, a pediatric IgAN, or a crescentic IgAN.

In an embodiment, the subject has or is identified as having a chronic kidney disease (CKD) or a disorder associated with CKD. In an embodiment, the CKD is an advanced CKD, e.g., with an estimated glomerular filtration rate (eGFR) equal to or greater than about 30 or about 45.

In an embodiment, the subject has or is identified as having a Henoch-Schonlein purpura (HSP).

In an embodiment, the subject has or is identified as having a cutaneous vasculitis or IgA vasculitis. In an embodiment, the subject has or is identified as having a IgA dermatitis, e.g., IgA bullous dermatosis. In an embodiment, the subject has or is identified as having a Waldenstrom macroglobulinemia (WM). In an embodiment, the subject has or is identified as having a lupus nephritis.

In an embodiment, the level of a-g IgA is determined in a sample from the subject. In an embodiment, the method further comprises determining the level of a-g IgA in a sample from the subject.

In an embodiment, the method further comprises determining the level of total IgA in the sample. In an embodiment, the method further comprising determining the level of IgM and/or IgG in the sample. In an embodiment, the method further comprises obtaining a sample from the subject. In an embodiment, the sample is a blood or serum sample.

In an embodiment, the subject has received, is receiving, or is going to receive, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject is, or is identified as being, in need of receiving, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject receives the vaccine before, concurrent with, or after administration of the anti-APRIL antibody molecule.

In an embodiment, administration of the anti-APRIL antibody molecule reduces the subject's ability to have an effective antigen-specific serum IgG and/or IgA response to the vaccine by no more than 5%, 10%, 15%, 20%, 25%, 30%, 35%, 40%, 45%, or 50%. In an embodiment, administration of the anti-APRIL antibody molecule does not reduce, or does not substantially reduce, the subject's ability to have an effective antigen-specific serum IgG and/or IgA response to the vaccine. In an embodiment, the subject has or maintains an effective (e.g., protective) antigen-specific serum IgG and/or IgA response to the vaccine after administration of the anti-APRIL antibody molecule.

In an embodiment, the vaccine comprises tetanus toxoid, diphtheria toxoid, or both (e.g., TENIVAC®). In an embodiment, the subject has or maintains an effective (e.g., protective) level of tetanus and/or diphtheria anti-toxoid IgG (e.g., equal to or above 0.1 IU/mL in the blood), e.g., 1, 2, 3, 4, 5, 6, 7, 8, 9, 10, 11, 12, 13, 14, 15, 16, or more weeks after administration of the anti-APRIL antibody molecule.

In another aspect, the disclosure features a method of selecting a subject for treating a disorder, the method comprising determining whether administration of an anti-APRIL antibody molecule reduces, or is likely to reduce, the level of a-g IgA (e.g., a-g IgA1) by at least 40% in a subject in need thereof, thereby selecting the subject. In an embodiment, the level of a-g IgA comprises, or is, the level of a-g IgA1.

In an embodiment, the anti-APRIL antibody molecule is an anti-APRIL antibody molecule described herein. In an embodiment, the anti-APRIL antibody molecule comprises the HCDR1, HCDR2, HCDR3, LCDR1, LCDR2, and LCDR3 of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237. In an embodiment, the anti-APRIL antibody molecule comprises the VH and VL of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237.

In an embodiment, the anti-APRIL antibody molecule reduces, or is likely to reduce, the level of a-g IgA by at least 40% about 4 weeks after the antibody molecule is administered. In an embodiment, the anti-APRIL antibody molecule reduces, or is likely to reduce, the level of a-g IgA by at least 40% about 8 weeks after the antibody molecule is administered. In an embodiment, the anti-APRIL antibody molecule reduces, or is likely to reduce, the level of a-g IgA by at least 40% about 12 weeks after the antibody molecule is administered. In an embodiment, the anti-APRIL antibody molecule reduces, or is likely to reduce, the level of a-g IgA by at least 40% about 16 weeks after the antibody molecule is administered. In an embodiment, the anti-APRIL antibody molecule reduces, or is likely to reduce, the level of a-g IgA by at least 40% for a predetermined period, e.g., at least one, two, three, or four weeks, or at least one, two, or three months. In an embodiment, the anti-APRIL antibody molecule reduces, or is likely to reduce, the level of a-g IgA by at least 50%. In an embodiment, the anti-APRIL antibody molecule reduces, or is likely to reduce, the level of a-g IgA by at least 55%, 60%, 65%, 70%, 75%, 80%, 85%, 90%, or 95%. In an embodiment, the antibody molecule is administered as a single dose. In an embodiment, the antibody molecule is administered as a repeated dose. In an embodiment, the antibody molecule is administered subcutaneously. In an embodiment, the antibody molecule is administered intravenously.

In an embodiment, the subject is a human. In an embodiment, the subject has, or is identified as having, a level of a-g IgA that is at least 1, 1.5, 2, 2.5, 3.5, 4, 4.5, or 5-fold higher than the level of a-g IgA in a reference subject, e.g., a subject who does not have the disorder, e.g., a healthy or normal subject. In an embodiment, the subject has, or is identified as having, a level of total IgA that is at least 1, 1.5, 2, 2.5, 3.5, 4, 4.5, or 5-fold higher than the level of total IgA in a reference subject, e.g., a subject who does not have the disorder, e.g., a healthy or normal subject. In an embodiment, the subject has received, or is receiving, a different therapeutic agent or modality for treating the disorder. In an embodiment, the subject has not received, or is not receiving, a different therapeutic agent or modality for treating the disorder. In an embodiment, the subject has, or is identified as having, a genomic susceptible locus of the disorder, e.g., IgA nephropathy. In an embodiment, the method further comprises determining whether the subject has a genomic susceptible locus of the disorder, e.g., IgA nephropathy.

In an embodiment, the subject has or is identified as having an APRIL-associated disorder. In an embodiment, the subject has or is identified as having a disorder associated with an aberrant level of total IgA. In an embodiment, the subject has or is identified as having a disorder associated with a-g IgA (e.g., a-g IgA1).

In an embodiment, the subject has or is identified as having an IgA nephropathy (IgAN). In an embodiment, the IgAN is a familial IgAN. In an embodiment, the IgA is an adult IgAN. In an embodiment, the IgAN is a post-transplant IgAN, a pediatric IgAN, or a crescentic IgAN.

In an embodiment, the subject has or is identified as having a chronic kidney disease (CKD) or a disorder associated with CKD. In an embodiment, the CKD is an advanced CKD, e.g., with an estimated glomerular filtration rate (eGFR) equal to or greater than about 30 or about 45.

In an embodiment, the subject has or is identified as having a Henoch-Schonlein purpura (HSP).

In an embodiment, the subject has or is identified as having a cutaneous vasculitis or IgA vasculitis. In an embodiment, the subject has or is identified as having an IgA dermatitis, e.g., IgA bullous dermatosis. In an embodiment, the subject has or is identified as having a Waldenstrom macroglobulinemia (WM). In an embodiment, the subject has or is identified as having a lupus nephritis.

In an embodiment, the level of a-g IgA is determined in a sample from the subject. In an embodiment, the method further comprises determining the level of a-g IgA in a sample from the subject.

In an embodiment, the method further comprises determining the level of total IgA in the sample. In an embodiment, the method further comprising determining the level of IgM and/or IgG in the sample. In an embodiment, the method further comprises obtaining a sample from the subject. In an embodiment, the sample is a blood or serum sample.

In an embodiment, the subject has received, is receiving, or is going to receive, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject is, or is identified as being, in need of receiving, a vaccine, e.g., within 1, 2, 3, 4, 5, or 6 days, or 1, 2, 3, 4, 5, 6, 7, 8, 9, or 10 weeks, of administration of the anti-APRIL antibody molecule. In an embodiment, the subject receives the vaccine before, concurrent with, or after administration of the anti-APRIL antibody molecule.

In an embodiment, administration of the anti-APRIL antibody molecule reduces the subject's ability to have an effective antigen-specific serum IgG and/or IgA response to the vaccine by no more than 5%, 10%, 15%, 20%, 25%, 30%, 35%, 40%, 45%, or 50%. In an embodiment, administration of the anti-APRIL antibody molecule does not reduce, or does not substantially reduce, the subject's ability to have an effective antigen-specific serum IgG and/or IgA response to the vaccine. In an embodiment, the subject has or maintains an effective (e.g., protective) antigen-specific serum IgG and/or IgA response to the vaccine after administration of the anti-APRIL antibody molecule.

In an embodiment, the vaccine comprises tetanus toxoid, diphtheria toxoid, or both (e.g., TENIVAC®). In an embodiment, the subject has or maintains an effective (e.g., protective) level of tetanus and/or diphtheria anti-toxoid IgG (e.g., equal to or above 0.1 IU/mL in the blood), e.g., 1, 2, 3, 4, 5, 6, 7, 8, 9, 10, 11, 12, 13, 14, 15, 16, or more weeks after administration of the anti-APRIL antibody molecule.

In another aspect, the disclosure features a method of treating IgA nephropathy, the method comprising administering to a subject in need thereof an effective amount of an anti-APRIL antibody molecule (e.g., an anti-APRIL antibody molecule described herein), wherein the subject has received, or is going to receive, a vaccine (e.g., a vaccine described herein) within 1, 2, 3, 4, 5, 6, 7, 8, 9, 10, 11, 12, 13, 14, or 15 weeks of administration of the anti-APRIL antibody molecule, thereby treating IgA nephropathy.

In an embodiment, the method further comprising administering the vaccine to the subject before, concurrently with, or after administration of the anti-APRIL antibody molecule.

In another aspect, the disclosure features a method of vaccinating a subject, the method comprising administering to the subject an effective amount of a vaccine (e.g., a vaccine described herein), wherein the subject has received, or is going to receive, an anti-APRIL antibody molecule (e.g., an anti-APRIL antibody molecule described herein) within 1, 2, 3, 4, 5, 6, 7, 8, 9, 10, 11, 12, 13, 14, or 15 weeks of administration of the vaccine, thereby vaccinating the subject.

In an embodiment, the method further comprising administering the anti-APRIL antibody molecule to the subject before, concurrent with, or after administration of the vaccine.

In yet another aspect, the disclosure features a method of treating a disorder, the method comprising administering an anti-APRIL antibody molecule to a subject in a need thereof, e.g., at a dose or dosage that reduces, or is likely to reduce, the level of IgM by at least a predetermined percentage in the subject, thereby treating the disorder.

In an embodiment, the anti-APRIL antibody molecule is an anti-APRIL antibody molecule described herein. In an embodiment, the anti-APRIL antibody molecule comprises the HCDR1, HCDR2, HCDR3, LCDR1, LCDR2, and LCDR3 of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237. In an embodiment, the anti-APRIL antibody molecule comprises the VH and VL of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237.

In an embodiment, the anti-APRIL antibody molecule reduces, or is likely to reduce, the level of IgM by at least 20%, 25%, 30%, 35%, 40%, 50%, 55%, 60%, 65%, 70%, 75%, 80%, 85%, 90%, or 95%.

In an embodiment, the antibody molecule is administered as a single dose. In an embodiment, the antibody molecule is administered as a repeated dose. In an embodiment, the antibody molecule is administered subcutaneously. In an embodiment, the antibody molecule is administered intravenously.

In an embodiment, the subject is a human. In an embodiment, the subject has, or is identified as having, a level of IgM that is at least 1, 1.5, 2, 2.5, 3.5, 4, 4.5, or 5-fold higher than the level of IgM in a reference subject, e.g., a subject who does not have the disorder, e.g., a healthy or normal subject. In an embodiment, the subject has received, or is receiving, a different therapeutic agent or modality for treating the disorder. In an embodiment, the subject has not received, or is not receiving, a different therapeutic agent or modality for treating the disorder.

In an embodiment, the disorder is associated an aberrant level of IgM. In an embodiment, the disorder is a chronic kidney disease (CKD) or kidney injury. In an embodiment, the disorder is a fibrosis.

In an embodiment, the disorder is an IgM mediated neuropathy, e.g., anti-MAG neuropathy or a neuropathy associated with anti-GM1. In an embodiment, the disorder is systemic lupus erythematosus (SLE). In an embodiment, the administration does not reduce, or does not substantially reduce, the level of IgG in the subject. In an embodiment, the administration reduces the level of IgG by no more than a predetermined percentage in the subject. In an embodiment, the administration reduces the level of IgG by at least a predetermined percentage in the subject.

In an embodiment, the level of IgM is determined in a sample from the subject. In an embodiment, the method further comprises determining the level of IgM in a sample from the subject. In an embodiment, the method further comprises determining the level of total IgM in the sample. In an embodiment, the method further comprising determining the level of IgA (e.g., total IgA and/or a-g IgA) and/or IgG in the sample. In an embodiment, the level of a-g IgA comprises, or is, the level of a-g IgA1.

In an embodiment, the method further comprises obtaining a sample from the subject. In an embodiment, the sample is a blood or serum sample.

In an embodiment, the method further comprises administering a second therapeutic agent or modality to the subject. In an embodiment, the second therapeutic agent or modality is a small molecule.

In an embodiment, the second therapeutic agent or modality is an antibody molecule.

In an embodiment, the subject has received, is receiving, or is going to receive, a vaccine, e.g., within 1, 2, 3, 4, 5, or 6 days, or 1, 2, 3, 4, 5, 6, 7, 8, 9, or 10 weeks, of administration of the antibody molecule. In an embodiment, the subject is, or is identified as being, in need of receiving, a vaccine, e.g., within 1, 2, 3, 4, 5, or 6 days, or 1, 2, 3, 4, 5, 6, 7, 8, 9, or 10 weeks, of administration of the antibody molecule. In an embodiment, the subject receives the vaccine before, concurrent with, or after administration of the antibody molecule.

In an embodiment, administration of the antibody molecule reduces the subject's ability to have an effective antigen-specific serum IgG and/or IgA response to the vaccine by no more than 5%, 10%, 15%, 20%, 25%, 30%, 35%, 40%, 45%, or 50%. In an embodiment, administration of the antibody molecule does not reduce, or does not substantially reduce, the subject's ability to have an effective antigen-specific serum IgG and/or IgA response to the vaccine. In an embodiment, the subject has or maintains an effective (e.g., protective) antigen-specific serum IgG and/or IgA response to the vaccine after the antibody molecule is administered.

In an embodiment, the vaccine comprises tetanus toxoid, diphtheria toxoid, or both (e.g., TENIVAC®). In an embodiment, the subject has or maintains an effective (e.g., protective) level of tetanus and/or diphtheria anti-toxoid IgG (e.g., equal to or above 0.1 IU/mL in the blood), e.g., 1, 2, 3, 4, 5, 6, 7, 8, 9, 10, 11, 12, 13, 14, 15, 16, or more weeks after the antibody molecule is administered.

In another aspect, the disclosure features a method of reducing the level of IgM in a subject, the method comprising administering an anti-APRIL antibody molecule to a subject in a need thereof, e.g., at a dose or dosage that reduces, or is likely to reduce, the level of IgM by at least a predetermined percentage in the subject, thereby reducing the level of IgM.

In an embodiment, the anti-APRIL antibody molecule is an anti-APRIL antibody molecule described herein. In an embodiment, the anti-APRIL antibody molecule comprises the HCDR1, HCDR2, HCDR3, LCDR1, LCDR2, and LCDR3 of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237. In an embodiment, the anti-APRIL antibody molecule comprises the VH and VL of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237.

In an embodiment, the anti-APRIL antibody molecule reduces, or is likely to reduce, the level of IgM by at least 20%, 25%, 30%, 35%, 40%, 50%, 55%, 60%, 65%, 70%, 75%, 80%, 85%, 90%, or 95%, e.g., over a predetermined period. In an embodiment, the antibody molecule is administered as a single dose. In an embodiment, the antibody molecule is administered as a repeated dose. In an embodiment, the antibody molecule is administered subcutaneously. In an embodiment, the antibody molecule is administered intravenously.

In an embodiment, the subject is a human. In an embodiment, the subject has, or is identified as having, a level of IgM that is at least 1, 1.5, 2, 2.5, 3.5, 4, 4.5, or 5-fold higher than the level of IgM in a reference subject, e.g., a subject who does not have the disorder, e.g., a healthy or normal subject. In an embodiment, the subject has received, or is receiving, a different therapeutic agent or modality for treating the disorder. In an embodiment, the subject has not received, or is not receiving, a different therapeutic agent or modality for treating the disorder.

In an embodiment, the disorder is associated an aberrant level of IgM. In an embodiment, the disorder is a chronic kidney disease (CKD) or kidney injury. In an embodiment, the disorder is a fibrosis.

In an embodiment, the disorder is an IgM mediated neuropathy, e.g., anti-MAG neuropathy or a neuropathy associated with anti-GM1. In an embodiment, the disorder is systemic lupus erythematosus (SLE). In an embodiment, the administration does not reduce, or does not substantially reduce, the level of IgG in the subject. In an embodiment, the administration reduces the level of IgG by no more than a predetermined percentage in the subject. In an embodiment, the administration reduces the level of IgG by at least a predetermined percentage in the subject.

In an embodiment, the level of IgM is determined in a sample from the subject. In an embodiment, the method further comprises determining the level of IgM in a sample from the subject. In an embodiment, the method further comprises determining the level of total IgM in the sample. In an embodiment, the method further comprising determining the level of IgA (e.g., total IgA and/or a-g IgA) and/or IgG in the sample. In an embodiment, the level of a-g IgA comprises, or is, the level of a-g IgA1. In an embodiment, the method further comprises obtaining a sample from the subject. In an embodiment, the sample is a blood or serum sample.

In an embodiment, the subject has received, is receiving, or is going to receive, a vaccine, e.g., within 1, 2, 3, 4, 5, or 6 days, or 1, 2, 3, 4, 5, 6, 7, 8, 9, or 10 weeks, of administration of the antibody molecule. In an embodiment, the subject is, or is identified as being, in need of receiving, a vaccine, e.g., within 1, 2, 3, 4, 5, or 6 days, or 1, 2, 3, 4, 5, 6, 7, 8, 9, or 10 weeks, of administration of the antibody molecule. In an embodiment, the subject receives the vaccine before, concurrent with, or after administration of the antibody molecule.

In an embodiment, administration of the antibody molecule reduces the subject's ability to have an effective antigen-specific serum IgG and/or IgA response to the vaccine by no more than 5%, 10%, 15%, 20%, 25%, 30%, 35%, 40%, 45%, or 50%. In an embodiment, administration of the antibody molecule does not reduce, or does not substantially reduce, the subject's ability to have an effective antigen-specific serum IgG and/or IgA response to the vaccine. In an embodiment, the subject has or maintains an effective (e.g., protective) antigen-specific serum IgG and/or IgA response to the vaccine after the antibody molecule is administered.

In an embodiment, the vaccine comprises tetanus toxoid, diphtheria toxoid, or both (e.g., TENIVAC®). In an embodiment, the subject has or maintains an effective (e.g., protective) level of tetanus and/or diphtheria anti-toxoid IgG (e.g., equal to or above 0.1 IU/mL in the blood), e.g., 1, 2, 3, 4, 5, 6, 7, 8, 9, 10, 11, 12, 13, 14, 15, 16, or more weeks after the antibody molecule is administered.

In another aspect, the disclosure features a method of treating a disorder, the method comprising administering an anti-APRIL antibody molecule to a subject in a need thereof, e.g., at a dose or dosage that reduces, or is likely to reduce, the levels of IgA and IgM by at least predetermined percentages in the subject, thereby treating the disorder.

In an embodiment, the level of IgA comprises, or is, the level of total IgA and/or a-g IgA. In an embodiment, the level of a-g IgA comprises, or is, the level of a-g IgA1.

In an embodiment, the anti-APRIL antibody molecule is an anti-APRIL antibody molecule described herein. In an embodiment, the anti-APRIL antibody molecule comprises the HCDR1, HCDR2, HCDR3, LCDR1, LCDR2, and LCDR3 of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237. In an embodiment, the anti-APRIL antibody molecule comprises the VH and VL of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237.

In an embodiment, the administration does not reduce or does not substantially reduce, the level of IgG in the subject. In an embodiment, the administration reduces the level of IgG by no more than a predetermined percentage in the subject. In an embodiment, the administration reduces the level of IgG by at least a predetermined percentage in the subject.

In an embodiment, the anti-APRIL antibody molecule reduces, or is likely to reduce, the level of a-g IgA by at least 20%, 25%, 30%, 35%, 40%, 45%, 50%, 55%, 60%, 65%, 70%, 75%, 80%, 85%, 90%, or 95%, e.g., over a predetermined period. In an embodiment, the anti-APRIL antibody molecule reduces, or is likely to reduce, the level of total IgA by at least 20%, 25%, 30%, 35%, 40%, 45%, 50%, 55%, 60%, 65%, 70%, 75%, 80%, 85%, 90%, or 95%, e.g., over a predetermined period. In an embodiment, the anti-APRIL antibody molecule reduces, or is likely to reduce, the level of IgM by at least 20%, 25%, 30%, 35%, 40%, 45%, 50%, 55%, 60%, 65%, 70%, 75%, 80%, 85%, 90%, or 95%, e.g., over a predetermined period. In an embodiment, the anti-APRIL antibody molecule reduces, or is likely to reduce, the level of IgA (e.g., total and/or a-g IgA) by at least 20%, 25%, 30%, 35%, 40%, 45%, 50%, 55%, 60%, 65%, 70%, 75%, 80%, 85%, 90%, or 95% and the level of IgM by at least 20%, 25%, 30%, 35%, 40%, 45%, 50%, 55%, 60%, 65%, 70%, 75%, 80%, 85%, 90%, or 95%, e.g., over a predetermined period. In an embodiment, the antibody molecule is administered as a single dose. In an embodiment, the antibody molecule is administered as a repeated dose. In an embodiment, the antibody molecule is administered subcutaneously. In an embodiment, the antibody molecule is administered intravenously.

In an embodiment, the subject is a human. In an embodiment, the subject has, or is identified as having, a level of a-g IgA that is at least 1, 1.5, 2, 2.5, 3.5, 4, 4.5, or 5-fold higher than the level of a-g IgA in a reference subject, e.g., a subject who does not have the disorder, e.g., a healthy or normal subject. In an embodiment, the subject has, or is identified as having, a level of total IgA that is at least 1, 1.5, 2, 2.5, 3.5, 4, 4.5, or 5-fold higher than the level of total IgA in a reference subject, e.g., a subject who does not have the disorder, e.g., a healthy or normal subject. In an embodiment, the subject has, or is identified as having, a level of IgM that is at least 1, 1.5, 2, 2.5, 3.5, 4, 4.5, or 5-fold higher than the level of a-g IgM in a reference subject, e.g., a subject who does not have the disorder, e.g., a healthy or normal subject. In an embodiment, the subject has received, or is receiving, a different therapeutic agent or modality for treating the disorder. In an embodiment, the subject has not received, or is not receiving, a different therapeutic agent or modality for treating the disorder.

In an embodiment, the disorder is an APRIL-associated disorder. In an embodiment, the disorder is associated an aberrant level of IgA (e.g., total IgA and/or a-g IgA) and/or IgM, e.g., a disorder described herein. In an embodiment, the disorder is systemic lupus erythematosus (SLE). In an embodiment, the administration does not reduce, or does not substantially reduce, the level of IgG in the subject. In an embodiment, the administration reduces the level of IgG by no more than a predetermined percentage in the subject. In an embodiment, the administration reduces the level of IgG by at least a predetermined percentage in the subject. In an embodiment, the level of IgA and/or IgM (and optionally IgG) is determined in a sample from the subject. In an embodiment, the method further comprises determining the level of a-g IgA in a sample from the subject. In an embodiment, the method further comprises determining the level of total IgA in the sample. In an embodiment, the method further comprising determining the level of IgM in the sample. In an embodiment, the method further comprising determining the level of IgG in the sample. In an embodiment, the method further comprises obtaining a sample from the subject. In an embodiment, the sample is a blood or serum sample.

In an embodiment, the method further comprises administering a second therapeutic agent or modality to the subject. In an embodiment, the second therapeutic agent or modality is a small molecule.

In an embodiment, the second therapeutic agent or modality is an antibody molecule.

In an embodiment, the subject has received, is receiving, or is going to receive, a vaccine, e.g., within 1, 2, 3, 4, 5, or 6 days, or 1, 2, 3, 4, 5, 6, 7, 8, 9, or 10 weeks, of administration of the antibody molecule. In an embodiment, the subject is, or is identified as being, in need of receiving, a vaccine, e.g., within 1, 2, 3, 4, 5, or 6 days, or 1, 2, 3, 4, 5, 6, 7, 8, 9, or 10 weeks, of administration of the antibody molecule. In an embodiment, the subject receives the vaccine before, concurrent with, or after administration of the antibody molecule.

In an embodiment, administration of the antibody molecule reduces the subject's ability to have an effective antigen-specific serum IgG and/or IgA response to the vaccine by no more than 5%, 10%, 15%, 20%, 25%, 30%, 35%, 40%, 45%, or 50%. In an embodiment, administration of the antibody molecule does not reduce, or does not substantially reduce, the subject's ability to have an effective antigen-specific serum IgG and/or IgA response to the vaccine. In an embodiment, the subject has or maintains an effective (e.g., protective) antigen-specific serum IgG and/or IgA response to the vaccine after the antibody molecule is administered.

In an embodiment, the vaccine comprises tetanus toxoid, diphtheria toxoid, or both (e.g., TENIVAC®). In an embodiment, the subject has or maintains an effective (e.g., protective) level of tetanus and/or diphtheria anti-toxoid IgG (e.g., equal to or above 0.1 IU/mL in the blood), e.g., 1, 2, 3, 4, 5, 6, 7, 8, 9, 10, 11, 12, 13, 14, 15, 16, or more weeks after the antibody molecule is administered.

In yet another aspect, the disclosure features a method of reducing the levels of IgA and IgM in subject, the method comprising administering an anti-APRIL antibody molecule to a subject in a need thereof, e.g., at a dose or dosage that reduces, or is likely to reduce, the levels of IgA and IgM by at least predetermined percentages in the subject, thereby reducing the levels of IgA and IgM.

In an embodiment, the level of IgA comprises, or is, the level of total IgA and/or a-g IgA. In an embodiment, the level of a-g IgA comprises, or is, the level of a-g IgA1.

In an embodiment, the anti-APRIL antibody molecule is an anti-APRIL antibody molecule described herein. In an embodiment, the anti-APRIL antibody molecule comprises the HCDR1, HCDR2, HCDR3, LCDR1, LCDR2, and LCDR3 of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237. In an embodiment, the anti-APRIL antibody molecule comprises the VH and VL of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237.

In an embodiment, the administration does not reduce or does not substantially reduce, the level of IgG in the subject. In an embodiment, the administration reduces the level of IgG by no more than a predetermined percentage in the subject. In an embodiment, the administration reduces the level of IgG by at least a predetermined percentage in the subject.

In an embodiment, the anti-APRIL antibody molecule reduces, or is likely to reduce, the level of a-g IgA by at least 20%, 25%, 30%, 35%, 40%, 50%, 55%, 60%, 65%, 70%, 75%, 80%, 85%, 90%, or 95%, e.g., over a predetermined period. In an embodiment, the anti-APRIL antibody molecule reduces, or is likely to reduce the level of total IgA by at least 20%, 25%, 30%, 35%, 40%, 50%, 55%, 60%, 65%, 70%, 75%, 80%, 85%, 90%, or 95%, e.g., over a predetermined period. In an embodiment, the anti-APRIL antibody molecule reduces, or is likely to reduce, the level of IgM by at least 20%, 25%, 30%, 35%, 40%, 50%, 55%, 60%, 65%, 70%, 75%, 80%, 85%, 90%, or 95%, e.g., over a predetermined period. In an embodiment, the anti-APRIL antibody molecule reduces, or is likely to reduce, the level of IgA (e.g., total and/or a-g IgA) by at least 20%, 25%, 30%, 35%, 40%, 45%, 50%, 55%, 60%, 65%, 70%, 75%, 80%, 85%, 90%, or 95% and the level of IgM by at least 20%, 25%, 30%, 35%, 40%, 45%, 50%, 55%, 60%, 65%, 70%, 75%, 80%, 85%, 90%, or 95%, e.g., over a predetermined period. In an embodiment, the antibody molecule is administered as a single dose. In an embodiment, the antibody molecule is administered as a repeated dose. In an embodiment, the antibody molecule is administered subcutaneously. In an embodiment, the antibody molecule is administered intravenously.

In an embodiment, the subject is a human. In an embodiment, the subject has, or is identified as having, a level of a-g IgA that is at least 1, 1.5, 2, 2.5, 3.5, 4, 4.5, or 5-fold higher than the level of a-g IgA in a reference subject, e.g., a subject who does not have the disorder, e.g., a healthy or normal subject. In an embodiment, the subject has, or is identified as having, a level of total IgA that is at least 1, 1.5, 2, 2.5, 3.5, 4, 4.5, or 5-fold higher than the level of total IgA in a reference subject, e.g., a subject who does not have the disorder, e.g., a healthy or normal subject. In an embodiment, the subject has, or is identified as having, a level of IgM that is at least 1, 1.5, 2, 2.5, 3.5, 4, 4.5, or 5-fold higher than the level of a-g IgM in a reference subject, e.g., a subject who does not have the disorder, e.g., a healthy or normal subject. In an embodiment, the subject has received, or is receiving, a different therapeutic agent or modality for treating the disorder. In an embodiment, the subject has not received, or is not receiving, a different therapeutic agent or modality for treating the disorder.

In an embodiment, the disorder is an APRIL-associated disorder. In an embodiment, the disorder is associated an aberrant level of IgA (e.g., total IgA and/or a-g IgA) and/or IgM, e.g., a disorder described herein. In an embodiment, the disorder is systemic lupus erythematosus (SLE). In an embodiment, the administration does not reduce, or does not substantially reduce, the level of IgG in the subject. In an embodiment, the administration reduces the level of IgG by no more than a predetermined percentage in the subject. In an embodiment, the administration reduces the level of IgG by at least a predetermined percentage in the subject. In an embodiment, the level of IgA and/or IgM (and optionally IgG) is determined in a sample from the subject. In an embodiment, the method further comprises determining the level of a-g IgA in a sample from the subject. In an embodiment, the method further comprises determining the level of total IgA in the sample. In an embodiment, the method further comprising determining the level of IgM in the sample. In an embodiment, the method further comprising determining the level of IgG in the sample. In an embodiment, the method further comprises obtaining a sample from the subject. In an embodiment, the sample is a blood or serum sample.

In an embodiment, the method further comprises administering a second therapeutic agent or modality to the subject. In an embodiment, the second therapeutic agent or modality is a small molecule.

In an embodiment, the second therapeutic agent or modality is an antibody molecule.

In an embodiment, the subject has received, is receiving, or is going to receive, a vaccine, e.g., within 1, 2, 3, 4, 5, or 6 days, or 1, 2, 3, 4, 5, 6, 7, 8, 9, or 10 weeks, of administration of the antibody molecule. In an embodiment, the subject is, or is identified as being, in need of receiving, a vaccine, e.g., within 1, 2, 3, 4, 5, or 6 days, or 1, 2, 3, 4, 5, 6, 7, 8, 9, or 10 weeks, of administration of the antibody molecule. In an embodiment, the subject receives the vaccine before, concurrent with, or after administration of the antibody molecule.

In an embodiment, administration of the antibody molecule reduces the subject's ability to have an effective antigen-specific serum IgG and/or IgA response to the vaccine by no more than 5%, 10%, 15%, 20%, 25%, 30%, 35%, 40%, 45%, or 50%. In an embodiment, administration of the antibody molecule does not reduce, or does not substantially reduce, the subject's ability to have an effective antigen-specific serum IgG and/or IgA response to the vaccine. In an embodiment, the subject has or maintains an effective (e.g., protective) antigen-specific serum IgG and/or IgA response to the vaccine after the antibody molecule is administered.

In an embodiment, the vaccine comprises tetanus toxoid, diphtheria toxoid, or both (e.g., TENIVAC®). In an embodiment, the subject has or maintains an effective (e.g., protective) level of tetanus and/or diphtheria anti-toxoid IgG (e.g., equal to or above 0.1 IU/mL in the blood), e.g., 1, 2, 3, 4, 5, 6, 7, 8, 9, 10, 11, 12, 13, 14, 15, 16, or more weeks after the antibody molecule is administered.

In another aspect, the disclosure features a method of treating a disorder, the method comprising administering to a subject in need thereof an effective amount of an anti-APRIL antibody molecule, wherein the disorder is:
(a) an advanced chronic kidney disease (CKD) (e.g., with an eGFR equal to or greater than about 30 or 45);
(b) a post-transplant IgAN;
(c) a pediatric IgAN;
(d) Henoch-Schonlein purpura (HSP) or cutaneous vasculitis;
(e) IgAN with crescentic glomerulonephritis (GN);
(f) IgA vasculitis;
(g) IgA dermatitis;
(h) IgM mediated neuropathy (anti-MAG or anti-GM1);
(i) Waldenstrom macroglobulinemia (WM); or
(j) lupus nephritis.

In an embodiment, the anti-APRIL antibody molecule is an anti-APRIL antibody molecule described herein. In an embodiment, the anti-APRIL antibody molecule comprises the HCDR1, HCDR2, HCDR3, LCDR1, LCDR2, and LCDR3 of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237. In an embodiment, the anti-APRIL antibody molecule comprises the VH and VL of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237.

In an embodiment, the administration reduces, or is likely to reduce, the IgA in the subject. In an embodiment, the administration reduces, or is likely to reduce, the IgM in the subject. In an embodiment, the level of IgA comprises, or is, the level of total IgA and/or a-g IgA. In an embodiment, the level of a-g IgA comprises, or is, the level of a-g IgA1.

In an embodiment, the administration does not reduce or does not substantially reduce, the level of IgG in the subject. In an embodiment, the administration reduces the level of IgG by no more than a predetermined percentage in the subject. In an embodiment, the administration reduces the level of IgG by at least a predetermined percentage in the subject.

In an embodiment, the anti-APRIL antibody molecule reduces, or is likely to reduce, the level of a-g IgA by at least 20%, 25%, 30%, 35%, 40%, 50%, 55%, 60%, 65%, 70%, 75%, 80%, 85%, 90%, or 95%, e.g., over a predetermined period. In an embodiment, the anti-APRIL antibody molecule reduces, or is likely to reduce the level of total IgA by at least 20%, 25%, 30%, 35%, 40%, 50%, 55%, 60%, 65%, 70%, 75%, 80%, 85%, 90%, or 95%, e.g., over a predetermined period. In an embodiment, the anti-APRIL antibody molecule reduces, or is likely to reduce, the level of IgM by at least 20%, 25%, 30%, 35%, 40%, 50%, 55%, 60%, 65%, 70%, 75%, 80%, 85%, 90%, or 95%, e.g., over a predetermined period. In an embodiment, the anti-APRIL antibody molecule reduces, or is likely to reduce, the level of IgA (e.g., total and/or a-g IgA) by at least 20%, 25%, 30%, 35%, 40%, 45%, 50%, 55%, 60%, 65%, 70%, 75%, 80%, 85%, 90%, or 95% and the level of IgM by at least 20%, 25%, 30%, 35%, 40%, 45%, 50%, 55%, 60%, 65%, 70%, 75%, 80%, 85%, 90%, or 95%, e.g., over a predetermined period. In an embodiment, the antibody molecule is administered as a single dose. In an embodiment, the antibody molecule is administered as a repeated dose. In an embodiment, the antibody molecule is administered subcutaneously. In an embodiment, the antibody molecule is administered intravenously.

In an embodiment, the subject is a human. In an embodiment, the subject has, or is identified as having, a level of a-g IgA that is at least 1, 1.5, 2, 2.5, 3.5, 4, 4.5, or 5-fold higher than the level of a-g IgA in a reference subject, e.g., a subject who does not have the disorder, e.g., a healthy or normal subject. In an embodiment, the subject has, or is identified as having, a level of total IgA that is at least 1, 1.5, 2, 2.5, 3.5, 4, 4.5, or 5-fold higher than the level of total IgA in a reference subject, e.g., a subject who does not have the disorder, e.g., a healthy or normal subject. In an embodiment, the subject has, or is identified as having, a level of IgM that is at least 1, 1.5, 2, 2.5, 3.5, 4, 4.5, or 5-fold higher than the level of a-g IgM in a reference subject, e.g., a subject who does not have the disorder, e.g., a healthy or normal subject. In an embodiment, the subject has received, or is receiving, a different therapeutic agent or modality for treating the disorder. In an embodiment, the subject has not received, or is not receiving, a different therapeutic agent or modality for treating the disorder.

In an embodiment, the disorder is an advanced chronic kidney disease (CKD) (e.g., with an eGFR equal to or greater than about 30 or 45). In an embodiment, the disorder is a post-transplant IgAN. In an embodiment, the disorder is a pediatric IgAN. In an embodiment, the disorder is Henoch-Schonlein purpura (HSP) or cutaneous vasculitis. In an embodiment, the disorder is IgAN with crescentic glomerulonephritis (GN). In an embodiment, the disorder is IgA vasculitis. In an embodiment, the disorder is IgA dermatitis. In an embodiment, the disorder is IgM mediated neuropathy (anti-MAG or anti-GM1). In an embodiment, the disorder is Waldenstrom macroglobulinemia (WM). In an embodiment, the disorder is lupus nephritis.

In an embodiment, the method further comprises administering a second therapeutic agent or modality to the subject. In an embodiment, the second therapeutic agent or modality is a small molecule.

In an embodiment, the second therapeutic agent or modality is an antibody molecule.

In an embodiment, the subject has received, is receiving, or is going to receive, a vaccine, e.g., within 1, 2, 3, 4, 5, or 6 days, or 1, 2, 3, 4, 5, 6, 7, 8, 9, or 10 weeks, of administration of the antibody molecule. In an embodiment, the subject is, or is identified as being, in need of receiving, a vaccine, e.g., within 1, 2, 3, 4, 5, or 6 days, or 1, 2, 3, 4, 5, 6, 7, 8, 9, or 10 weeks, of administration of the antibody molecule. In an embodiment, the subject receives the vaccine before, concurrent with, or after administration of the antibody molecule.

In an embodiment, administration of the antibody molecule reduces the subject's ability to have an effective antigen-specific serum IgG and/or IgA response to the vaccine by no more than 5%, 10%, 15%, 20%, 25%, 30%, 35%, 40%, 45%, or 50%. In an embodiment, administration of the antibody molecule does not reduce, or does not substantially reduce, the subject's ability to have an effective antigen-specific serum IgG and/or IgA response to the vaccine. In an embodiment, the subject has or maintains an effective (e.g., protective) antigen-specific serum IgG and/or IgA response to the vaccine after the antibody molecule is administered.

In an embodiment, the vaccine comprises tetanus toxoid, diphtheria toxoid, or both (e.g., TENIVAC®). In an embodiment, the subject has or maintains an effective (e.g., protective) level of tetanus and/or diphtheria anti-toxoid IgG (e.g., equal to or above 0.1 IU/mL in the blood), e.g., 1, 2, 3, 4, 5, 6, 7, 8, 9, 10, 11, 12, 13, 14, 15, 16, or more weeks after the antibody molecule is administered.

In another aspect, the disclosure features a method of treating a disorder associated with an autoantigen, the method comprising administering to a subject in need thereof an effective amount of a therapeutic agent or modality, wherein the administration reduces, or is likely to reduce, the level of autoantigen by at least a predetermined percentage in the subject.

In an embodiment, the subject is a human. In an embodiment, the subject has or is identified as having an APRIL-associated disorder.

In an embodiment, the level of the autoantigen is determined in a sample from the subject. embodiment, the method further comprises obtaining a sample from the subject. In an embodiment, the sample is a blood or serum sample.

In an embodiment, the method further comprises administering a second therapeutic agent or modality to the subject. In an embodiment, the second therapeutic agent or modality is a small molecule.

In an embodiment, the second therapeutic agent or modality is an antibody molecule.

In an aspect, the disclosure features a method of treating IgA nephropathy, the method comprising:

responsive to an identification of a subject who will benefit from administration of an anti-APRIL antibody molecule, administering to the subject an effective amount of the anti-APRIL antibody molecule,

wherein the anti-APRIL antibody molecule is administered no more than once a month,

wherein the benefit comprises one or more (e.g., two, three, or all) of the following:
(i) reduction of the level of APRIL by 90% or more, within one-month administration of the anti-APRIL antibody molecule, compared to the subject's baseline level of APRIL;
(ii) reduction of the level of galactose-deficient IgA1 (Gd-IgA1) by 60% or more, within nine-month administration of the anti-APRIL antibody molecule, compared to the subject's baseline level of Gd-IgA;
(iii) reduction of 24-hour urine protein creatinine ratio (uPCR) by 30% or more, within nine-month administration of the anti-APRIL antibody molecule, compared to the subject's baseline uPCR; or
(iv) preserving (e.g., maintaining or increasing) average eGFR over a period of at least 12 months after administration of the anti-APRIL antibody molecule, compared to the subject's baseline eGFR,

thereby treating IgA nephropathy.

In an embodiment, the anti-APRIL antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3), and

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16.

In an embodiment, the subject's baseline level of APRIL is the level of APRIL prior to administration (e.g., prior to the first administration) of the anti-APRIL antibody molecule. In an embodiment, the subject's baseline level of Gd-IgA is the level of Gd-IgA prior to administration (e.g., prior to the first administration) of the anti-APRIL antibody molecule. In an embodiment, the subject's baseline uPCR is the uPCR prior to administration (e.g., prior to the first administration) of the anti-APRIL antibody molecule. In an embodiment, the subject's baseline eGFR is the eGFR prior to administration (e.g., prior to the first administration) of the anti-APRIL antibody molecule.

In an embodiment, the method further comprises identifying a subject who will benefit from administration of the anti-APRIL antibody molecule.

In an embodiment, the benefit comprises: (ii) reduction of the level of galactose-deficient IgA1 (Gd-IgA1) by 60% or more, within nine-month administration of the anti-APRIL antibody molecule, compared to the subject's baseline level of Gd-IgA; and (iv) preserving (e.g., maintaining or increasing) average eGFR over a period of at least 12 months after administration of the anti-APRIL antibody molecule, compared to the subject's baseline eGFR.

In an embodiment, the level of APRIL is reduced by 91%, 92%, 93%, 94%, 95%, 96%, 97%, 98%, 99%, or more, within one-month administration of the anti-APRIL antibody molecule, compared to the subject's baseline level of APRIL. In an embodiment, the level of Gd-IgA1 is reduced by 65%, 70%, 75%, 85%, 90%, 95%, or more, within nine-month administration of the anti-APRIL antibody molecule, compared to the subject's baseline level of Gd-IgA. In an embodiment, the 24-hour uPCR is reduced by 35%, 40%, 45%, 50%, 55%, 60%, or more, within nine-month administration of the anti-APRIL antibody molecule, compared to the subject's baseline uPCR. In an embodiment, the average eGFR is preserved (e.g., maintained or increased) over a period of at least 13, 14, 15, 16, 17, 18, 19, 20, 21, 22, 23, or 24 months, compared to the subject's baseline eGFR.

In an embodiment, the benefit further comprises (v) reduction of the level of IgG, compared to the subject's baseline level of IgG, optionally wherein the level of IgG is the level of anti-gd-IgA1 IgG. In an embodiment, the benefit further comprises (vi) reduction of the level of IgA, compared to the subject's baseline level of IgA. In an embodiment, the benefit further comprises (vii) the ratio of average eGFR over a period of 12 months after administration of the anti-APRIL antibody molecule, relative to the expected average eGFR over a period of 12 months if the subject had not been administered the anti-APRIL antibody molecule, is at least 2, 3, 4, 5, 6, 7, 8, 9, 10, 11, 12, 13, 14, or 15. In an embodiment, the benefit further comprises (vii) not having a decrease in eGFR by 5%, 10%, 15%, or more, within one-month administration of the anti-APRIL antibody molecule, compared to the subject's baseline eGFR.

In an embodiment, the subject has one or more (e.g., two or all) of the following, prior to administration of the anti-APRIL antibody molecule, (a) received an angiotensin-converting enzyme (ACE) inhibitor or angiotensin receptor blocker (ARB) for at least three month; (b) a uPCR greater than 0.75 g/g or a level of 24-hour urine protein (UP) greater than 1.0 g/d; or (c) an eGFR greater than 30 mL/min/1.73 m². In an embodiment, the subject has one or both of the following, prior to administration of the anti-APRIL antibody molecule, (a) a proteinuria level greater than 2.0 g/day; or (b) an eGFR equal to or less than 60 mL/min/1.73 m².

In an embodiment, the anti-APRIL antibody molecule comprises a VH comprising the amino acid sequence of SEQ ID NO: 296, an amino acid sequence having at least 85%, 90%, 95%, 96%, 97%, 98%, or 99% identity thereto, or an amino acid sequence differing by no more than 1, 2, 3, 4, 5, 6, 7, 8, 9, or 10 amino acids therefrom. In an embodiment, the anti-APRIL antibody molecule comprises a VL comprising the amino acid sequence of SEQ ID NO: 286, or an amino acid sequence having at least 85%, 90%, 95%, 96%, 97%, 98%, or 99% identity thereto, or an amino acid sequence differing by no more than 1, 2, 3, 4, 5, 6, 7, 8, 9, or 10 amino acids therefrom. In an embodiment, the anti-APRIL antibody molecule comprises a VH comprising the amino acid sequence of SEQ ID NO: 296, and a VL comprising the amino acid sequence of SEQ ID NO: 286. In an embodiment, the anti-APRIL antibody molecule comprises a heavy chain constant region of IgG2 and a light chain constant region of kappa. In an embodiment, the anti-APRIL antibody molecule is sibeprenlimab.

In an embodiment, the anti-APRIL antibody molecule is administered at a dose of 2 mg/kg, 4 mg/kg, or 8 mg/kg. In an embodiment, the anti-APRIL antibody molecule is administered at a dose of 2 mg/kg. In an embodiment, the anti-APRIL antibody molecule is administered at a dose of 4 mg/kg. In an embodiment, the anti-APRIL antibody molecule is administered at a dose of 8 mg/kg. In an embodiment, the anti-APRIL antibody molecule is administered once a month, once every two months, once every three months, or once every six months. In an embodiment, the anti-APRIL antibody molecule is administered repeatedly, e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 times. In an embodiment, the anti-APRIL antibody molecule is administered once a month for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In an embodiment, the anti-APRIL antibody molecule is administered intravenously or subcutaneously. In an embodiment, the anti-APRIL antibody molecule is administered intravenously. In an embodiment, the anti-APRIL antibody molecule is administered subcutaneously.

In an aspect, the disclosure features a method of treating IgA nephropathy, the method comprising administering to a subject in need thereof an effective amount of an anti-APRIL antibody molecule, wherein the anti-APRIL antibody molecule is administered no more than once a month, wherein the administration results in one or more (e.g., two, three, or all) of the following:
(i) reduction of the level of APRIL by 90% or more, within one-month administration of the anti-APRIL antibody molecule, compared to the subject's baseline level of APRIL;
(ii) reduction of the level of galactose-deficient IgA1 (Gd-IgA1) by 60% or more, within nine-month administration of the anti-APRIL antibody molecule, compared to the subject's baseline level of Gd-IgA;
(iii) reduction of 24-hour urine protein creatinine ratio (uPCR) by 30% or more, within nine-month administration of the anti-APRIL antibody molecule, compared to the subject's baseline uPCR; or
(iv) preserving (e.g., maintaining or increasing) average eGFR over a period of at least 12 months after administration of the anti-APRIL antibody molecule, compared to the subject's baseline eGFR, thereby treating IgA nephropathy.

In an embodiment, the anti-APRIL antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3), and

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16.

In an embodiment, the subject's baseline level of APRIL is the level of APRIL prior to administration (e.g., prior to the first administration) of the anti-APRIL antibody molecule. In an embodiment, the subject's baseline level of Gd-IgA is the level of Gd-IgA prior to administration (e.g., prior to the first administration) of the anti-APRIL antibody molecule. In an embodiment, the subject's baseline uPCR is the uPCR prior to administration (e.g., prior to the first administration) of the anti-APRIL antibody molecule. In an embodiment, the subject's baseline eGFR is the eGFR prior to administration (e.g., prior to the first administration) of the anti-APRIL antibody molecule.

In an embodiment, the benefit comprises, or the administration results in: (ii) reduction of the level of galactose-deficient IgA1 (Gd-IgA1) by 60% or more, within nine-month administration of the anti-APRIL antibody molecule, compared to the subject's baseline level of Gd-IgA; and (iv) preserving (e.g., maintaining or increasing) average eGFR over a period of at least 12 months after administration of the anti-APRIL antibody molecule, compared to the subject's baseline eGFR.

In an embodiment, the level of APRIL is reduced by 91%, 92%, 93%, 94%, 95%, 96%, 97%, 98%, 99%, or more, within one-month administration of the anti-APRIL antibody molecule, compared to the subject's baseline level of APRIL. In an embodiment, the level of Gd-IgA1 is reduced by 65%, 70%, 75%, 85%, 90%, 95%, or more, within nine-month administration of the anti-APRIL antibody molecule, compared to the subject's baseline level of Gd-IgA. In an embodiment, the 24-hour uPCR is reduced by 35%, 40%, 45%, 50%, 55%, 60%, or more, within nine-month administration of the anti-APRIL antibody molecule, compared to the subject's baseline uPCR. In an embodiment, the average eGFR is preserved (e.g., maintained or increased) over a period of at least 13, 14, 15, 16, 17, 18, 19, 20, 21, 22, 23, or 24 months, compared to the subject's baseline eGFR.

In an embodiment, the administration further results in (v) reduction of the level of IgG, compared to the subject's baseline level of IgG, optionally wherein the level of IgG is the level of anti-gd-IgA1 IgG. In an embodiment, the administration further results in (vi) reduction of the level of IgA, compared to the subject's baseline level of IgA. In an embodiment, the administration further results in (vii) the ratio of average eGFR over a period of 12 months after administration of the anti-APRIL antibody molecule, relative to the expected average eGFR over a period of 12 months if the subject had not been administered the anti-APRIL antibody molecule, is at least 2, 3, 4, 5, 6, 7, 8, 9, 10, 11, 12, 13, 14, or 15. In an embodiment, the administration further results in, (vii) not having a decrease in eGFR by 5%, 10%, 15%, or more, within one-month administration of the anti-APRIL antibody molecule, compared to the subject's baseline eGFR.

In an embodiment, the subject has one or more (e.g., two or all) of the following, prior to administration of the anti-APRIL antibody molecule, (a) received an angiotensin-converting enzyme (ACE) inhibitor or angiotensin receptor blocker (ARB) for at least three month; (b) a uPCR greater than 0.75 g/g or a level of 24-hour urine protein (UP) greater than 1.0 g/d; or (c) an eGFR greater than 30 mL/min/1.73 m². In an embodiment, the subject has one or both of the following, prior to administration of the anti-APRIL antibody molecule, (a) a proteinuria level greater than 2.0 g/day; or (b) an eGFR equal to or less than 60 mL/min/1.73 m².

In an embodiment, the anti-APRIL antibody molecule comprises a VH comprising the amino acid sequence of SEQ ID NO: 296, an amino acid sequence having at least 85%, 90%, 95%, 96%, 97%, 98%, or 99% identity thereto, or an amino acid sequence differing by no more than 1, 2, 3, 4, 5, 6, 7, 8, 9, or 10 amino acids therefrom. In an embodiment, the anti-APRIL antibody molecule comprises a VL comprising the amino acid sequence of SEQ ID NO: 286, or an amino acid sequence having at least 85%, 90%, 95%, 96%, 97%, 98%, or 99% identity thereto, or an amino acid sequence differing by no more than 1, 2, 3, 4, 5, 6, 7, 8, 9, or 10 amino acids therefrom. In an embodiment, the anti-APRIL antibody molecule comprises a VH comprising the amino acid sequence of SEQ ID NO: 296, and a VL comprising the amino acid sequence of SEQ ID NO: 286. In an embodiment, the anti-APRIL antibody molecule comprises a heavy chain constant region of IgG2 and a light chain constant region of kappa. In an embodiment, the anti-APRIL antibody molecule is sibeprenlimab.

In an embodiment, the anti-APRIL antibody molecule is administered at a dose of 2 mg/kg, 4 mg/kg, or 8 mg/kg. In an embodiment, the anti-APRIL antibody molecule is administered at a dose of 2 mg/kg. In an embodiment, the anti-APRIL antibody molecule is administered at a dose of 4 mg/kg. In an embodiment, the anti-APRIL antibody molecule is administered at a dose of 8 mg/kg. In an embodiment, the anti-APRIL antibody molecule is administered once a month, once every two months, once every three months, or once every six months. In an embodiment, the anti-APRIL antibody molecule is administered repeatedly, e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 times. In an embodiment, the anti-APRIL antibody molecule is administered once a month for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In an embodiment, the anti-APRIL antibody molecule is administered intravenously or subcutaneously. In an embodiment, the anti-APRIL antibody molecule is administered intravenously. In an embodiment, the anti-APRIL antibody molecule is administered subcutaneously.

In an aspect, the disclosure features a method of treating IgA nephropathy, the method comprising administering to a subject in need thereof an effective amount of an anti-APRIL antibody molecule, wherein the subject has, or is identified to have, (a) a proteinuria level greater than 2.0 g/day, (b) an estimated glomerular filtration rate (eGFR) less than 60 mL/min/1.73 m², or (c) both (a) and (b), optionally, wherein the anti-APRIL antibody molecule is administered no more than once a month, thereby treating IgA nephropathy.

In an embodiment, the anti-APRIL antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3), and

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16.

In an embodiment, the anti-APRIL antibody molecule is administered responsive to an identification of subject who has (a) a proteinuria level greater than 2.0 g/day, (b) an estimated glomerular filtration rate (eGFR) less than 60 mL/min/1.73 m², or (c) both (a) and (b).

In an embodiment, the method further comprises identifying a subject who has (a) a proteinuria level greater than 2.0 g/day, (b) an estimated glomerular filtration rate (eGFR) less than 60 mL/min/1.73 m², or (c) both (a) and (b).

In an embodiment, the anti-APRIL antibody molecule comprises a VH comprising the amino acid sequence of SEQ ID NO: 296, an amino acid sequence having at least 85%, 90%, 95%, 96%, 97%, 98%, or 99% identity thereto, or an amino acid sequence differing by no more than 1, 2, 3, 4, 5, 6, 7, 8, 9, or 10 amino acids therefrom. In an embodiment, the anti-APRIL antibody molecule comprises a VL comprising the amino acid sequence of SEQ ID NO: 286, or an amino acid sequence having at least 85%, 90%, 95%, 96%, 97%, 98%, or 99% identity thereto, or an amino acid sequence differing by no more than 1, 2, 3, 4, 5, 6, 7, 8, 9, or 10 amino acids therefrom. In an embodiment, the anti-APRIL antibody molecule comprises a VH comprising the amino acid sequence of SEQ ID NO: 296, and a VL comprising the amino acid sequence of SEQ ID NO: 286. In an embodiment, the anti-APRIL antibody molecule comprises a heavy chain constant region of IgG2 and a light chain constant region of kappa. In an embodiment, the anti-APRIL antibody molecule is sibeprenlimab.

In an embodiment, the anti-APRIL antibody molecule is administered at a dose of 2 mg/kg, 4 mg/kg, or 8 mg/kg. In an embodiment, the anti-APRIL antibody molecule is administered at a dose of 2 mg/kg. In an embodiment, the anti-APRIL antibody molecule is administered at a dose of 4 mg/kg. In an embodiment, the anti-APRIL antibody molecule is administered at a dose of 8 mg/kg. In an embodiment, the anti-APRIL antibody molecule is administered once a month, once every two months, once every three months, or once every six months. In an embodiment, the anti-APRIL antibody molecule is administered repeatedly, e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 times. In an embodiment, the anti-APRIL antibody molecule is administered once a month for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In an embodiment, the anti-APRIL antibody molecule is administered intravenously or subcutaneously. In an embodiment, the anti-APRIL antibody molecule is administered intravenously. In an embodiment, the anti-APRIL antibody molecule is administered subcutaneously.

In an aspect, the disclosure features a method of treating IgA nephropathy, comprising administering to a subject in need thereof an effective amount of a second anti-APRIL antibody molecule,

wherein the subject has been administered with a first anti-APRIL antibody molecule at a dose of at least 600 mg once every two weeks, and the administration of the first anti-APRIL antibody is discontinued,

wherein the second anti-APRIL antibody molecule is administered no more than once a month,

thereby treating IgA nephropathy.

In some embodiments, the second anti-APRIL antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3), and

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16.

In an embodiment, the administration of the first anti-APRIL antibody molecule is discontinued and the second anti-APRIL antibody molecule is administered, responsive to an identification of a subject who has been administered with a first anti-APRIL antibody molecule at a dose of at least 600 mg once every two weeks.

In an embodiment, the method further comprises: (a) identifying a subject who has been administered with a first anti-APRIL antibody molecule at a dose of at least 600 mg once every two weeks; and (b) discontinuing the administration of the first anti-APRIL antibody molecule.

In an embodiment, the second anti-APRIL antibody molecule comprises a VH comprising the amino acid sequence of SEQ ID NO: 296, an amino acid sequence having at least 85%, 90%, 95%, 96%, 97%, 98%, or 99% identity thereto, or an amino acid sequence differing by no more than 1, 2, 3, 4, 5, 6, 7, 8, 9, or 10 amino acids therefrom. In an embodiment, the second anti-APRIL antibody molecule comprises a VL comprising the amino acid sequence of SEQ ID NO: 286, or an amino acid sequence having at least 85%, 90%, 95%, 96%, 97%, 98%, or 99% identity thereto, or an amino acid sequence differing by no more than 1, 2, 3, 4, 5, 6, 7, 8, 9, or 10 amino acids therefrom. In an embodiment, the second anti-APRIL antibody molecule comprises a VH comprising the amino acid sequence of SEQ ID NO: 296, and a VL comprising the amino acid sequence of SEQ ID NO: 286. In an embodiment, the second anti-APRIL antibody molecule comprises a heavy chain constant region of IgG2 and a light chain constant region of kappa. In an embodiment, the second anti-APRIL antibody molecule is sibeprenlimab.

In an embodiment, the second anti-APRIL antibody molecule is administered at a dose of 2 mg/kg, 4 mg/kg, or 8 mg/kg. In an embodiment, the second anti-APRIL antibody molecule is administered at a dose of 2 mg/kg. In an embodiment, the second anti-APRIL antibody molecule is administered at a dose of 4 mg/kg. In an embodiment, the second anti-APRIL antibody molecule is administered at a dose of 8 mg/kg. In an embodiment, the second anti-APRIL antibody molecule is administered once a month, once every two months, once every three months, or once every six months. In an embodiment, the second anti-APRIL antibody molecule is administered repeatedly, e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 times. In an embodiment, the second anti-APRIL antibody molecule is administered once a month for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In an embodiment, the second anti-APRIL antibody molecule is administered intravenously or subcutaneously. In an embodiment, the second anti-APRIL antibody molecule is administered intravenously. In an embodiment, the second anti-APRIL antibody molecule is administered subcutaneously.

In an embodiment, the first anti-APRIL antibody molecule is BION-1301 or any one of the anti-APRIL antibody molecules disclosed in WO2010/100056, WO2015/034364, WO2016/110587 and WO2021/243298.

In an aspect, the disclosure features a method of treating IgA nephropathy, the method comprising administering to a subject in need thereof a second anti-APRIL antibody molecule at a dose of at least 600 mg once every two weeks,

wherein the subject has been administered with a first anti-APRIL antibody molecule, and the administration of the first anti-APRIL antibody molecule is discontinued,

thereby treating IgA nephropathy.

In some embodiments, the first anti-APRIL antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3), and

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16.

In an embodiment, the second anti-APRIL antibody molecule is administered, responsive to an identification of a subject who has been administered with a first anti-APRIL antibody molecule.

In an embodiment, the method further comprises: (a) identifying a subject who has been administered with a first anti-APRIL antibody molecule; and (b) discontinuing the administration of the first anti-APRIL antibody molecule.

In an embodiment, the first anti-APRIL antibody molecule comprises a VH comprising the amino acid sequence of SEQ ID NO: 296, an amino acid sequence having at least 85%, 90%, 95%, 96%, 97%, 98%, or 99% identity thereto, or an amino acid sequence differing by no more than 1, 2, 3, 4, 5, 6, 7, 8, 9, or 10 amino acids therefrom. In an embodiment, the first anti-APRIL antibody molecule comprises a VL comprising the amino acid sequence of SEQ ID NO: 286, or an amino acid sequence having at least 85%, 90%, 95%, 96%, 97%, 98%, or 99% identity thereto, or an amino acid sequence differing by no more than 1, 2, 3, 4, 5, 6, 7, 8, 9, or 10 amino acids therefrom. In an embodiment, the first anti-APRIL antibody molecule comprises a VH comprising the amino acid sequence of SEQ ID NO: 296, and a VL comprising the amino acid sequence of SEQ ID NO: 286. In an embodiment, the first anti-APRIL antibody molecule comprises a heavy chain constant region of IgG2 and a light chain constant region of kappa. In an embodiment, the first anti-APRIL antibody molecule is sibeprenlimab.

In an embodiment, the first anti-APRIL antibody molecule is administered at a dose of 2 mg/kg, 4 mg/kg, or 8 mg/kg. In an embodiment, the first anti-APRIL antibody molecule is administered at a dose of 2 mg/kg. In an embodiment, the first anti-APRIL antibody molecule is administered at a dose of 4 mg/kg. In an embodiment, the first anti-APRIL antibody molecule is administered at a dose of 8 mg/kg. In an embodiment, the first anti-APRIL antibody molecule is administered once a month, once every two months, once every three months, or once every six months. In an embodiment, the first anti-APRIL antibody molecule is administered repeatedly, e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 times. In an embodiment, the first anti-APRIL antibody molecule is administered once a month for a period of at least 1, 2, 3, 4, 5, 6,7, 8,9, 10, 11, 12, 13, 14, 15, 16, 17, 18, 19, 20, 21, 22, 23, 24, 25, 26, 27, 28, 29, 30, 31, 32, 33, 34, 35, or 36 months. In an embodiment, the first anti-APRIL antibody molecule is administered intravenously or subcutaneously. In an embodiment, the first anti-APRIL antibody molecule is administered intravenously. In an embodiment, the first anti-APRIL antibody molecule is administered subcutaneously.

In an embodiment, the second anti-APRIL antibody molecule is BION-1301 or any one of the anti-APRIL antibody molecule disclosed in WO2010/100056, WO2015/034364, WO2016/110587 and WO2021/243298.

In an aspect, the disclosure features a method of treating IgA nephropathy, comprising administering to a subject in need thereof an effective amount of an anti-APRIL antibody molecule,

wherein the subject has been administered with (i) budesonide (e.g., at a dose of 16 mg once daily) (ii) atrasentan (e.g., at a dose of 0.75 mg once daily), (iii) dapagliflozin (e.g., at a dose of 5 mg or 10 mg once daily); or (iv) methylprednisolone (e.g., once daily), and the administration of (i) budesonide, (ii) atrasentan, (iii) dapagliflozin, or (iv) methylprednisolone, is discontinued,

wherein the anti-APRIL antibody molecule is administered no more than once a month,

thereby treating IgA nephropathy.

In some embodiments, the anti-APRIL antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3), and

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16.

In an embodiment, the anti-APRIL antibody molecule is administered, responsive to an identification of a subject who has been administered with (i) budesonide (e.g., at a dose of 16 mg once daily), (ii) atrasentan (e.g., at a dose of 0.75 mg once daily), (iii) dapagliflozin (e.g., at a dose of 5 mg or 10 mg once daily); or (iv) methylprednisolone (e.g., once daily).

In an embodiment, the method further comprises: (a) identifying a subject who has been administered with (i) budesonide (e.g., at a dose of 16 mg once daily), (ii) atrasentan (e.g., at a dose of 0.75 mg once daily), (iii) dapagliflozin (e.g., at a dose of 5 mg or 10 mg once daily); or (iv) methylprednisolone (e.g., once daily); and (b) discontinuing the administration of (i) budesonide, (ii) atrasentan, (iii) dapagliflozin, or (iv) methylprednisolone.

In an embodiment, the anti-APRIL antibody molecule comprises a VH comprising the amino acid sequence of SEQ ID NO: 296, an amino acid sequence having at least 85%, 90%, 95%, 96%, 97%, 98%, or 99% identity thereto, or an amino acid sequence differing by no more than 1, 2, 3, 4, 5, 6, 7, 8, 9, or 10 amino acids therefrom. In an embodiment, the anti-APRIL antibody molecule comprises a VL comprising the amino acid sequence of SEQ ID NO: 286, or an amino acid sequence having at least 85%, 90%, 95%, 96%, 97%, 98%, or 99% identity thereto, or an amino acid sequence differing by no more than 1, 2, 3, 4, 5, 6, 7, 8, 9, or 10 amino acids therefrom. In an embodiment, the anti-APRIL antibody molecule comprises a VH comprising the amino acid sequence of SEQ ID NO: 296, and a VL comprising the amino acid sequence of SEQ ID NO: 286. In an embodiment, the anti-APRIL antibody molecule comprises a heavy chain constant region of IgG2 and a light chain constant region of kappa. In an embodiment, the anti-APRIL antibody molecule is sibeprenlimab.

In an embodiment, the anti-APRIL antibody molecule is administered at a dose of 2 mg/kg, 4 mg/kg, or 8 mg/kg. In an embodiment, the anti-APRIL antibody molecule is administered at a dose of 2 mg/kg. In an embodiment, the anti-APRIL antibody molecule is administered at a dose of 4 mg/kg. In an embodiment, the anti-APRIL antibody molecule is administered at a dose of 8 mg/kg. In an embodiment, the anti-APRIL antibody molecule is administered once a month, once every two months, once every three months, or once every six months. In an embodiment, the anti-APRIL antibody molecule is administered repeatedly, e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 times. In an embodiment, the anti-APRIL antibody molecule is administered once a month for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In an embodiment, the anti-APRIL antibody molecule is administered intravenously or subcutaneously. In an embodiment, the anti-APRIL antibody molecule is administered intravenously. In an embodiment, the anti-APRIL antibody molecule is administered subcutaneously.

In an aspect, the disclosure features a method of treating IgA nephropathy, the method comprising administering to a subject in need thereof (i) budesonide (e.g., at a dose of 16 mg once daily) (ii) atrasentan (e.g., at a dose of 0.75 mg once daily), (iii) dapagliflozin (e.g., at a dose of 5 mg or 10 mg once daily); or (iv) methylprednisolone (e.g., once daily),

wherein the subject has been administered with an anti-APRIL antibody molecule, and the administration of the anti-APRIL antibody molecule is discontinued,

thereby treating IgA nephropathy.

In some embodiments, the first anti-APRIL antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3), and

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16.

In an embodiment, (i) budesonide, (ii) atrasentan, (iii) dapagliflozin, or (iv) methylprednisolone is administered, responsive to an identification of a subject who has been administered with an anti-APRIL antibody molecule.

In an embodiment, the method further comprises: (a) identifying a subject who has been administered with an anti-APRIL antibody molecule; (b) discontinuing the administration of the anti-APRIL antibody molecule.

In an embodiment, the anti-APRIL antibody molecule comprises a VH comprising the amino acid sequence of SEQ ID NO: 296, an amino acid sequence having at least 85%, 90%, 95%, 96%, 97%, 98%, or 99% identity thereto, or an amino acid sequence differing by no more than 1, 2, 3, 4, 5, 6, 7, 8, 9, or 10 amino acids therefrom. In an embodiment, the anti-APRIL antibody molecule comprises a VL comprising the amino acid sequence of SEQ ID NO: 286, or an amino acid sequence having at least 85%, 90%, 95%, 96%, 97%, 98%, or 99% identity thereto, or an amino acid sequence differing by no more than 1, 2, 3, 4, 5, 6, 7, 8, 9, or 10 amino acids therefrom. In an embodiment, the anti-APRIL antibody molecule comprises a VH comprising the amino acid sequence of SEQ ID NO: 296, and a VL comprising the amino acid sequence of SEQ ID NO: 286. In an embodiment, the anti-APRIL antibody molecule comprises a heavy chain constant region of IgG2 and a light chain constant region of kappa. In an embodiment, the anti-APRIL antibody molecule is sibeprenlimab.

In an embodiment, the anti-APRIL antibody molecule is administered at a dose of 2 mg/kg, 4 mg/kg, or 8 mg/kg. In an embodiment, the anti-APRIL antibody molecule is administered at a dose of 2 mg/kg. In an embodiment, the anti-APRIL antibody molecule is administered at a dose of 4 mg/kg. In an embodiment, the anti-APRIL antibody molecule is administered at a dose of 8 mg/kg. In an embodiment, the anti-APRIL antibody molecule is administered once a month, once every two months, once every three months, or once every six months. In an embodiment, the anti-APRIL antibody molecule is administered repeatedly, e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 times. In an embodiment, the anti-APRIL antibody molecule is administered once a month for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In an embodiment, the anti-APRIL antibody molecule is administered intravenously or subcutaneously. In an embodiment, the anti-APRIL antibody molecule is administered intravenously. In an embodiment, the anti-APRIL antibody molecule is administered subcutaneously.

In an aspect, the disclosure features a method of selecting a subject for a therapy comprising an anti-APRIL antibody molecule, the method comprising:
(a) determining whether a subject will have one or more (e.g., two, three, or all) of the following after administration of the anti-APRIL antibody molecule:
   (i) reduction of the level of APRIL by 90% or more, within one-month administration of the anti-APRIL antibody molecule, compared to the subject's baseline level of APRIL;
   (ii) reduction of the level of galactose-deficient IgA1 (Gd-IgA1) by 60% or more, within nine-month administration of the anti-APRIL antibody molecule, compared to the subject's baseline level of Gd-IgA;
   (iii) reduction of 24-hour urine protein creatinine ratio (uPCR) by 30% or more, within nine-month administration of the anti-APRIL antibody molecule, compared to the subject's baseline uPCR; or
   (iv) preserving (e.g., maintaining or increasing) average eGFR over a period of at least 12 months after administration of the anti-APRIL antibody molecule, compared to the subject's baseline eGFR;
(b) selecting the subject based on the determination that the subject will have one or more (e.g., two, three, or all) of (i), (ii), (iii), or (iv) after administration of the anti-APRIL antibody molecule, wherein the anti-APRIL antibody molecule is to be administered no more than once a month, wherein the subject has IgA nephropathy, or is at risk of having IgA nephropathy, thereby selecting the subject.

In an embodiment, the anti-APRIL antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3), and

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16.

In an embodiment, the subject's baseline level of APRIL is the level of APRIL prior to administration (e.g., prior to the first administration) of the anti-APRIL antibody molecule. In an embodiment, the subject's baseline level of Gd-IgA is the level of Gd-IgA prior to administration (e.g., prior to the first administration) of the anti-APRIL antibody molecule. In an embodiment, the subject's baseline uPCR is the uPCR prior to administration (e.g., prior to the first administration) of the anti-APRIL antibody molecule. In an embodiment, the subject's baseline eGFR is the eGFR prior to administration (e.g., prior to the first administration) of the anti-APRIL antibody molecule.

In an embodiment, determining whether a subject will have one or more (e.g., two, three, or all) of (i), (ii), (iii), or (iv) after administration of the anti-APRIL antibody molecule is based, at least in part, on a study described in Example 10.

In an embodiment, the anti-APRIL antibody molecule comprises a VH comprising the amino acid sequence of SEQ ID NO: 296, an amino acid sequence having at least 85%, 90%, 95%, 96%, 97%, 98%, or 99% identity thereto, or an amino acid sequence differing by no more than 1, 2, 3, 4, 5, 6, 7, 8, 9, or 10 amino acids therefrom. In an embodiment, the anti-APRIL antibody molecule comprises a VL comprising the amino acid sequence of SEQ ID NO: 286, or an amino acid sequence having at least 85%, 90%, 95%, 96%, 97%, 98%, or 99% identity thereto, or an amino acid sequence differing by no more than 1, 2, 3, 4, 5, 6, 7, 8, 9, or 10 amino acids therefrom. In an embodiment, the anti-APRIL antibody molecule comprises a VH comprising the amino acid sequence of SEQ ID NO: 296, and a VL comprising the amino acid sequence of SEQ ID NO: 286. In an embodiment, the anti-APRIL antibody molecule comprises a heavy chain constant region of IgG2 and a light chain constant region of kappa. In an embodiment, the anti-APRIL antibody molecule is sibeprenlimab.

In an embodiment, the anti-APRIL antibody molecule is to be administered at a dose of 2 mg/kg, 4 mg/kg, or 8 mg/kg. In an embodiment, the anti-APRIL antibody molecule is to be administered at a dose of 2 mg/kg. In an embodiment, the anti-APRIL antibody molecule is to be administered at a dose of 4 mg/kg. In an embodiment, the anti-APRIL antibody molecule is to be administered at a dose of 8 mg/kg. In an embodiment, the anti-APRIL antibody molecule is to be administered once a month, once every two months, once every three months, or once every six months. In an embodiment, the anti-APRIL antibody molecule is to be administered repeatedly, e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 times. In an embodiment, the anti-APRIL antibody molecule is to be administered once a month for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In an embodiment, the anti-APRIL antibody molecule is to be administered intravenously or subcutaneously. In an embodiment, the anti-APRIL antibody molecule is to be administered intravenously. In an embodiment, the anti-APRIL antibody molecule is to be administered subcutaneously. In an embodiment, the method further comprises administering the anti-APRIL antibody molecule to the subject.

In an aspect, the disclosure features a method of selecting a therapy comprising an anti-APRIL antibody molecule for a subject, the method comprising:
(a) determining whether a subject will have one or more (e.g., two, three, or all) of the following after administration of the anti-APRIL antibody molecule:
   (i) reduction of the level of APRIL by 90% or more, within one-month administration of the anti-APRIL antibody molecule, compared to the subject's baseline level of APRIL;
   (ii) reduction of the level of galactose-deficient IgA1 (Gd-IgA1) by 60% or more, within nine-month administration of the anti-APRIL antibody molecule, compared to the subject's baseline level of Gd-IgA;
   (iii) reduction of 24-hour urine protein creatinine ratio (uPCR) by 30% or more, within nine-month administration of the anti-APRIL antibody molecule, compared to the subject's baseline uPCR; or
   (iv) preserving (e.g., maintaining or increasing) average eGFR over a period of at least 12 months after administration of the anti-APRIL antibody molecule, compared to the subject's baseline eGFR;
(b) selecting the therapy comprising the anti-APRIL antibody molecule based on the determination that the subject will have one or more (e.g., two, three, or all) of (i), (ii), (iii), or (iv) after administration of the anti-APRIL antibody molecule,

wherein the anti-APRIL antibody molecule is administered no more than once a month,

wherein the subject has IgA nephropathy, or is at risk of having IgA nephropathy,

thereby selecting the therapy.

In an embodiment, the anti-APRIL antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3), and

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16.

In an embodiment, the subject's baseline level of APRIL is the level of APRIL prior to administration (e.g., prior to the first administration) of the anti-APRIL antibody molecule. In an embodiment, the subject's baseline level of Gd-IgA is the level of Gd-IgA prior to administration (e.g., prior to the first administration) of the anti-APRIL antibody molecule. In an embodiment, the subject's baseline uPCR is the uPCR prior to administration (e.g., prior to the first administration) of the anti-APRIL antibody molecule. In an embodiment, the subject's baseline eGFR is the eGFR prior to administration (e.g., prior to the first administration) of the anti-APRIL antibody molecule.

In an embodiment, determining whether a subject will have one or more (e.g., two, three, or all) of (i), (ii), (iii), or (iv) after administration of the anti-APRIL antibody molecule is based, at least in part, on a study described in Example 10.

In an embodiment, the anti-APRIL antibody molecule comprises a VH comprising the amino acid sequence of SEQ ID NO: 296, an amino acid sequence having at least 85%, 90%, 95%, 96%, 97%, 98%, or 99% identity thereto, or an amino acid sequence differing by no more than 1, 2, 3, 4, 5, 6, 7, 8, 9, or 10 amino acids therefrom. In an embodiment, the anti-APRIL antibody molecule comprises a VL comprising the amino acid sequence of SEQ ID NO: 286, or an amino acid sequence having at least 85%, 90%, 95%, 96%, 97%, 98%, or 99% identity thereto, or an amino acid sequence differing by no more than 1, 2, 3, 4, 5, 6, 7, 8, 9, or 10 amino acids therefrom. In an embodiment, the anti-APRIL antibody molecule comprises a VH comprising the amino acid sequence of SEQ ID NO: 296, and a VL comprising the amino acid sequence of SEQ ID NO: 286. In an embodiment, the anti-APRIL antibody molecule comprises a heavy chain constant region of IgG2 and a light chain constant region of kappa. In an embodiment, the anti-APRIL antibody molecule is sibeprenlimab.

In an embodiment, the anti-APRIL antibody molecule is to be administered at a dose of 2 mg/kg, 4 mg/kg, or 8 mg/kg. In an embodiment, the anti-APRIL antibody molecule is to be administered at a dose of 2 mg/kg. In an embodiment, the anti-APRIL antibody molecule is to be administered at a dose of 4 mg/kg. In an embodiment, the anti-APRIL antibody molecule is to be administered at a dose of 8 mg/kg. In an embodiment, the anti-APRIL antibody molecule is to be administered once a month, once every two months, once every three months, or once every six months. In an embodiment, the anti-APRIL antibody molecule is to be administered repeatedly, e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 times. In an embodiment, the anti-APRIL antibody molecule is to be administered once a month for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In an embodiment, the anti-APRIL antibody molecule is to be administered intravenously or subcutaneously. In an embodiment, the anti-APRIL antibody molecule is to be administered intravenously. In an embodiment, the anti-APRIL antibody molecule is to be administered subcutaneously. In an embodiment, the method further comprises administering the anti-APRIL antibody molecule to the subject.

### Enumerated Embodiments

1. A method of treating a disorder, comprising:

administering to a subject in need thereof an anti-APRIL antibody molecule described herein,

wherein the antibody molecule is administered at a dose that reduces, or is likely to reduce, the level of aberrantly glycosylated IgA (a-g IgA) by at least 40% (e.g., by at least 40%, 50%, 60%, 70%, 75%, 80%, 85%, 90%, 95%, 96%, 97%, 98%, 99%, or 100%) in the subject,

thereby treating the disorder.

2. A method of treating a disorder, comprising:

administering to a subject in need thereof an anti-APRIL antibody molecule described herein,

wherein the administration reduces the level of a-g IgA by at least 40% (e.g., by at least 40%, 50%, 60%, 70%, 75%, 80%, 85%, 90%, 95%, 96%, 97%, 98%, 99%, or 100%) in the subject,

thereby treating the disorder.

3. A method of treating a disorder, comprising:

administering to a subject in need thereof an anti-APRIL antibody molecule described herein,

wherein the antibody molecule is administered at a dosage (e.g., dose and frequency) that reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in the subject,

thereby treating the disorder.

4. A method of treating a disorder, comprising:

selecting a dose or dosage (e.g., dose and frequency) for an anti-APRIL antibody molecule described herein, wherein administration of the antibody molecule at the dose or dosage reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in a subject in need thereof, and

administering the antibody molecule to the subject at the selected dose or dosage,

thereby treating the disorder.

5. A method of treating a disorder, comprising:

responsive to a determination that administration of an anti-APRIL antibody molecule described herein reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in a subject in need thereof, administering to the subject an anti-APRIL antibody molecule,

thereby treating the disorder.

6. A method of treating a disorder, comprising:

determining whether administration of an anti-APRIL antibody molecule described herein reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in a subject in need thereof,

if the antibody molecule reduces, or is likely to reduce, the level of a-g IgA by at least 40%, administration of the antibody molecule is initiated, continued, or maintained,

optionally wherein if the antibody molecule does not reduce, or is not likely to reduce, the level of a-g IgA by at least 40%, administration of the antibody molecule is terminated, discontinued, or altered, and/or a different therapeutic agent or modality is administered.

7. A method of treating a disorder, comprising:

determining whether administration of an anti-APRIL antibody molecule described herein at a dose or dosage reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in a subject in need thereof,

if the antibody molecule reduces, or is likely to reduce, the level of a-g IgA by at least 40% at the dose or dosage, administration of the antibody molecule at the dose or dosage is initiated, continued, or maintained,

optionally wherein if the antibody molecule does not reduce, or is not likely to reduce, the level of a-g IgA by at least 40% at the dose or dosage, administration of the antibody molecule at the dose or dosage is terminated, discontinued, or altered.

8. A method of treating a disorder, comprising:

determining whether administration of a therapeutic agent or modality other than an anti-APRIL antibody molecule described herein reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in a subject in need thereof,

if the therapeutic agent or modality does not reduce, or is not likely to reduce, the level of a-g IgA by at least 40%, administering an anti-APRIL antibody molecule described herein to the subject.

9. A method of reducing the level of a-g IgA in subject, comprising:

administering an anti-APRIL antibody molecule described herein to a subject in a need thereof, e.g., at a dose or dosage that reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in the subject,

thereby reducing the level of a-g IgA.

10. A method of selecting an anti-APRIL antibody molecule for treating a disorder, comprising:

determining whether administration of an anti-APRIL antibody molecule described herein reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in a subject in need thereof,

thereby selecting the anti-APRIL antibody molecule.

11. A method of selecting a dose or dosage (e.g., dose and frequency) for an anti-APRIL antibody molecule for treating a disorder, comprising:

determining whether administration of an anti-APRIL antibody molecule described herein at a dose or dosage reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in a subject in need thereof,

thereby selecting the dose or dosage.

12. A method of selecting a subject for treating a disorder, comprising:

determining whether administration of an anti-APRIL antibody molecule described herein reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in a subject in need thereof,

thereby selecting the subject,

optionally wherein if the antibody molecule does not reduce, or is not likely to reduce, the level of a-g IgA by at least 40%, administration of the antibody molecule is terminated, discontinued, or altered, or a different therapeutic agent or modality is administered.

13. The method of any of embodiments 1-12, wherein the a-g IgA comprises or is a-g IgA1.

14. The method of any of embodiments 1-13, wherein the level of a-g IgA is reduced by at least 40% (e.g., by at least 40%, 50%, 60%, 70%, 75%, 80%, 85%, 90%, 95%, 96%, 97%, 98%, 99%, or 100%) for a predetermined period, e.g., at least one, two, three, or four weeks, or at least one, two, or three months.

15. The method of any of embodiments 1-14, wherein the level of a-g IgA is reduced by at least 40% (e.g., by at least 40%, 50%, 60%, 70%, 75%, 80%, 85%, 90%, 95%, 96%, 97%, 98%, 99%, or 100%) about 4 weeks after the antibody molecule is administered.

16. The method of any of embodiments 1-15, wherein the level of a-g IgA is reduced by at least 40% (e.g., by at least 40%, 50%, 60%, 70%, 75%, 80%, 85%, 90%, 95%, 96%, 97%, 98%, 99%, or 100%) about 8 weeks after the antibody molecule is administered.

17. The method of any of embodiments 1-16, wherein the level of a-g IgA is reduced by at least 40% (e.g., by at least 40%, 50%, 60%, 70%, 75%, 80%, 85%, 90%, 95%, 96%, 97%, 98%, 99%, or 100%) about 12 weeks after the antibody molecule is administered.

18. The method of any of embodiments 1-17, wherein the level of a-g IgA is reduced by at least 40% (e.g., by at least 40%, 50%, 60%, 70%, 75%, 80%, 85%, 90%, 95%, 96%, 97%, 98%, 99%, or 100%) about 16 weeks after the antibody molecule is administered.

19. The method of any of embodiments 1-18, wherein the level of a-g IgA is reduced by at least 50%.

20. The method of any of embodiments 1-19, wherein the level of a-g IgA is reduced by at least 55%, 60%, 65%, 70%, 75%, 80%, 85%, 90%, or 95%.

21. The method of any of embodiments 1-20, wherein the antibody molecule is administered as a single dose.

22. The method of any of embodiments 1-20, wherein the antibody molecule is administered as a repeated dose.

23. The method of any of embodiments 1-22, wherein the antibody molecule is administered subcutaneously.

24. The method of any of embodiments 1-22, wherein the antibody molecule is administered intravenously.

25. The method of any of embodiments 1-24, wherein the disorder is an APRIL-associated disorder.

26. The method of any of embodiments 1-25, wherein the disorder is associated with an aberrant level of total IgA.

27. The method of any of embodiments 1-26, wherein the disorder is a disorder associated with a-g IgA.

28. The method of any of embodiments 1-27, wherein the disorder is IgA nephropathy (IgAN).

29. The method of embodiment 28, wherein the IgAN is a familial IgAN.

30. The method of embodiment 28, wherein the IgAN is an adult IgAN.

31. The method of embodiment 28, wherein the IgAN is a post-transplant IgAN, a pediatric IgAN, or a crescentic IgAN.

32. The method of any of embodiments 1-27, wherein the disorder is a chronic kidney disease (CKD) or a disorder associated with CKD.

33. The method of embodiment 32, wherein the CKD is an advanced CKD, e.g., with an estimated glomerular filtration rate (eGFR) equal to or greater than about 30 or about 45.

34. The method of any of embodiments 1-27, wherein the disorder is Henoch-Schonlein purpura (HSP).

35. The method of any of embodiments 1-27, wherein the disorder is cutaneous vasculitis or IgA vasculitis.

36. The method of any of embodiments 1-27, wherein the disorder is IgA dermatitis, e.g., IgA bullous dermatosis.

37. The method of any of embodiments 1-27, wherein the disorder is Waldenstrom macroglobulinemia (WM).

38. The method of any of embodiments 1-27, wherein the disorder is lupus nephritis.

39. The method of any of embodiments 1-38, wherein the subject is a human.

40. The method of any of embodiments 1-39, wherein the subject has, or is identified as having, a level of a-g IgA that is at least 1, 1.5, 2, 2.5, 3.5, 4, 4.5, or 5-fold higher than the level of a-g IgA in a reference subject, e.g., a subject who does not have the disorder, e.g., a healthy or normal subject.

41. The method of any of embodiments 1-40, wherein the subject has, or is identified as having, a level of total IgA that is at least 1, 1.5, 2, 2.5, 3.5, 4, 4.5, or 5-fold higher than the level of total IgA in a reference subject, e.g., a subject who does not have the disorder, e.g., a healthy or normal subject.

42. The method of any of embodiments 1-41, wherein the subject has received, or is receiving, a different therapeutic agent or modality for treating the disorder.

43. The method of any of embodiments 1-41, wherein the subject has not received, or is not receiving, a different therapeutic agent or modality for treating the disorder.

44. The method of any of embodiments 1-43, wherein the subject has received, is receiving, or is going to receive, a vaccine, e.g., within 1, 2, 3, 4, 5, or 6 days, or 1, 2, 3, 4, 5, 6, 7, 8, 9, or 10 weeks, of administration of the antibody molecule.

45. The method of any of embodiments 1-43, wherein the subject is, or is identified as being, in need of receiving, a vaccine, e.g., within 1, 2, 3, 4, 5, or 6 days, or 1, 2, 3, 4, 5, 6, 7, 8, 9, or 10 weeks, of administration of the antibody molecule.

46. The method of embodiment 44 or 45, wherein the subject receives the vaccine before, concurrent with, or after administration of the antibody molecule.

47. The method of any of embodiments 44-46, wherein administration of the antibody molecule reduces the subject's ability to have an effective antigen-specific serum IgG and/or IgA response to the vaccine by no more than 5%, 10%, 15%, 20%, 25%, 30%, 35%, 40%, 45%, or 50%.

48. The method of any of embodiments 44-47, wherein administration of the antibody molecule does not reduce, or does not substantially reduce, the subject's ability to have an effective antigen-specific serum IgG and/or IgA response to the vaccine.

49. The method of any of embodiments 44-48, wherein the subject has or maintains an effective (e.g., protective) antigen-specific serum IgG and/or IgA response to the vaccine after administration of the antibody molecule.

50. The method of any of embodiments 44-49, wherein the vaccine comprises tetanus toxoid, diphtheria toxoid, or both (e.g., TENIVAC®).

51. The method of embodiment 50, wherein the subject has or maintains an effective (e.g., protective) level of tetanus and/or diphtheria anti-toxoid IgG (e.g., equal to or above 0.1 IU/mL in the blood), e.g., 1, 2, 3, 4, 5, 6, 7, 8, 9, 10, 11, 12, 13, 14, 15, 16, or more weeks after administration of the antibody molecule.

52. The method of any of embodiments 1-51, wherein the subject has, or is identified as having, a genomic susceptible locus of the disorder, e.g., IgA nephropathy.

53. The method of any of embodiments 1-52, further comprising determining whether the subject has a genomic susceptible locus of the disorder, e.g., IgA nephropathy.

54. The method of any of embodiments 1-53, wherein the antibody molecule comprises the HCDR1, HCDR2, HCDR3, LCDR1, LCDR2, and LCDR3 of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237.

55. The method of any of embodiments 1-54, wherein the antibody molecule comprises the VH and VL of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237.

56. The method of any of embodiments 1-55, wherein the level of a-g IgA is determined in a sample from the subject.

57. The method of any of embodiments 1-56, further comprising determining the level of a-g IgA in a sample from the subject.

58. The method of any of embodiments 1-57, further comprising determining the level of total IgA in the sample.

59. The method of any of embodiments 1-58, further comprising determining the level of IgM and/or IgG in the sample.

60. The method of any of embodiments 1-59, further comprising obtaining a sample from the subject.

61. The method of embodiment 60, wherein the sample is a blood or serum sample.

62. The method of any of embodiments 1-61, further comprising administering a second therapeutic agent or modality to the subject.

63. The method of embodiment 62, wherein the second therapeutic agent or modality is a small molecule.

64. The method of embodiment 62, wherein the second therapeutic agent or modality is an antibody molecule.

65. A method of treating IgA nephropathy, comprising:

administering to a subject in need thereof an effective amount of an anti-APRIL antibody molecule (e.g., an anti-APRIL antibody molecule described herein),

wherein the subject has received, or is going to receive, a vaccine (e.g., a vaccine described herein) within 1, 2, 3, 4, 5, 6, 7, 8, 9, 10, 11, 12, 13, 14, or 15 weeks of administration of the antibody molecule,

thereby treating IgA nephropathy.

66. The method of embodiment 65, further comprising administering the vaccine to the subject before, concurrent with, or after administration of the antibody molecule.

67. A method of vaccinating a subject, comprising:

administering to the subject an effective amount of a vaccine (e.g., a vaccine described herein),

wherein the subject has received, or is going to receive, an anti-APRIL antibody molecule (e.g., an anti-APRIL antibody molecule described herein) within 1, 2, 3, 4, 5, 6, 7, 8, 9, 10, 11, 12, 13, 14, or 15 weeks of administration of the vaccine,

thereby vaccinating the subject.

68. The method of embodiment 67, further comprising administering the antibody molecule to the subject before, concurrent with, or after administration of the vaccine.

69. The method of any of embodiments 44-68, wherein the vaccine is administered intramuscularly.

70. A composition for use in treating IgA nephropathy in a subject, wherein the composition comprises an anti-APRIL antibody molecule (e.g., an anti-APRIL antibody molecule described herein) at a dosage of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg,

wherein the subject has received, or is going to receive, a vaccine (e.g., a vaccine described herein) within 1, 2, 3, 4, 5, 6, 7, 8, 9, 10, 11, 12, 13, 14, or 15 weeks of administration of the antibody molecule.

71. The composition for use of embodiment 70, further wherein the subject has been administered the vaccine before, concurrent with, or after administration of the antibody molecule.

72. A composition for use in vaccinating a subject, the composition comprising an effective amount of a vaccine (e.g., a vaccine described herein),

wherein the subject has received, or is going to receive, an anti-APRIL antibody molecule (e.g., an anti-APRIL antibody molecule described herein) within 1, 2, 3, 4, 5, 6, 7, 8, 9, 10, 11, 12, 13, 14, or 15 weeks of administration of the vaccine, wherein the subject received, or is going to receive, the anti-APRIL antibody molecule at a dosage of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg.

73. The composition for use of embodiment 72, wherein the subject is administered the antibody molecule before, concurrent with, or after administration of the vaccine.

74. A composition for use in treating a disorder in a subject, the composition comprising:

an anti-APRIL antibody molecule described herein at a dosage of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg; and

wherein the dosage reduces, or is likely to reduce, the level of aberrantly glycosylated IgA (a-g IgA) by at least 40% (e.g., by at least 40%, 50%, 60%, 70%, 75%, 80%, 85%, 90%, 95%, 96%, 97%, 98%, 99%, or 100%) in the subject.

75. A composition for use in treating a disorder in a subject, the composition comprising an anti-APRIL antibody molecule described herein at a dosage of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg,

wherein the dosage reduces the level of a-g IgA by at least 40% (e.g., by at least 40%, 50%, 60%, 70%, 75%, 80%, 85%, 90%, 95%, 96%, 97%, 98%, 99%, or 100%) in the subject.

76. A composition for use in treating a disorder in a subject, the composition comprising an anti-APRIL antibody molecule described herein at a dosage (e.g., dose and frequency) that reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in the subject,

wherein the dosage is about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg.

77. A composition for use in treating a disorder in a subject, the composition comprising an anti-APRIL antibody molecule described herein to the subject at a dosage of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg;

wherein the composition is formulated administered if administration of a therapeutic agent or modality other than an anti-APRIL antibody molecule described herein reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in the subject.

78. A composition for use in reducing the level of a-g IgA in a subject, the composition comprising an anti-APRIL antibody molecule described herein to a subject in a need thereof at a dose or dosage that reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in the subject,

wherein the dose or dosage is about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg.

79. A method of treating IgA nephropathy, comprising:

administering to a subject in need thereof an anti-APRIL antibody molecule (e.g., an anti-APRIL antibody molecule described herein) at a dosage of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg,

wherein the subject has received, or is going to receive, a vaccine (e.g., a vaccine described herein) within 1, 2, 3, 4, 5, 6, 7, 8, 9, 10, 11, 12, 13, 14, or 15 weeks of administration of the antibody molecule,

thereby treating IgA nephropathy.

80. The method of embodiment 79, further comprising administering the vaccine to the subject before, concurrent with, or after administration of the antibody molecule.

81. A method of vaccinating a subject, comprising:

administering to the subject an effective amount of a vaccine (e.g., a vaccine described herein),

wherein the subject has received, or is going to receive, an anti-APRIL antibody molecule (e.g., an anti-APRIL antibody molecule described herein) within 1, 2, 3, 4, 5, 6, 7, 8, 9, 10, 11, 12, 13, 14, or 15 weeks of administration of the vaccine, wherein the subject received, or is going to receive, the anti-APRIL antibody molecule at a dosage of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg;

thereby vaccinating the subject.

82. The method of embodiment 81, further comprising administering the antibody molecule to the subject before, concurrent with, or after administration of the vaccine.

83. A method of treating a disorder, comprising:

administering to a subject in need thereof an anti-APRIL antibody molecule described herein,

wherein the antibody molecule is administered at a dosage of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg; and

wherein the dosage administered to the subject reduces, or is likely to reduce, the level of aberrantly glycosylated IgA (a-g IgA) by at least 40% (e.g., by at least 40%, 50%, 60%, 70%, 75%, 80%, 85%, 90%, 95%, 96%, 97%, 98%, 99%, or 100%) in the subject,

thereby treating the disorder.

84. A method of treating a disorder, comprising:

administering to a subject in need thereof an anti-APRIL antibody molecule described herein at a dosage of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg,

wherein the administration reduces the level of a-g IgA by at least 40% (e.g., by at least 40%, 50%, 60%, 70%, 75%, 80%, 85%, 90%, 95%, 96%, 97%, 98%, 99%, or 100%) in the subject,

thereby treating the disorder.

85. A method of treating a disorder, comprising:

administering to a subject in need thereof an anti-APRIL antibody molecule described herein,

wherein the antibody molecule is administered at a dosage (e.g., dose and frequency) that reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in the subject, and

wherein the dosage is about 0.5 mg/kg, 2.0 mg/kg, 6 mg/kg, 9 mg/kg, 9.1 mg/kg, 12 mg/kg, or at

a fixed dose of about 200 mg, 400 mg, 600 mg, or 800 mg;

thereby treating the disorder.

86. A method of treating a disorder, comprising:

selecting a dose or dosage (e.g., dose and frequency) for an anti-APRIL antibody molecule described herein,
wherein the dose or dosage is about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg; and
wherein administration of the antibody molecule at the dose or dosage reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in a subject in need thereof, and administering the antibody molecule to the subject at the selected dose or dosage, thereby treating the disorder.

87. A method of treating a disorder, comprising:

responsive to a determination that administration of an anti-APRIL antibody molecule described herein reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in a subject in need thereof, administering to the subject an anti-APRIL antibody molecule at a dosage of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg,

thereby treating the disorder.

88. A method of treating a disorder, comprising:

determining whether administration of an anti-APRIL antibody molecule described herein reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in a subject in need thereof,

if the antibody molecule reduces, or is likely to reduce, the level of a-g IgA by at least 40%, administration of the antibody molecule at a dosage of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg is initiated, continued, or maintained,

optionally wherein if the antibody molecule does not reduce, or is not likely to reduce, the level of a-g IgA by at least 40%, administration of the antibody molecule is terminated, discontinued, or altered, and/or a different therapeutic agent or modality is administered.

89. A method of treating a disorder, comprising:

determining whether administration of an anti-APRIL antibody molecule described herein at a dose or dosage reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in a subject in need thereof,

if the antibody molecule reduces, or is likely to reduce, the level of a-g IgA by at least 40% at the dose or dosage, administration of the antibody molecule at the dose or dosage is initiated, continued, or maintained,

wherein the dose or dosage is about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg;

optionally wherein if the antibody molecule does not reduce, or is not likely to reduce, the level of a-g IgA by at least 40% at the dose or dosage, administration of the antibody molecule at the dose or dosage is terminated, discontinued, or altered.

90. A method of treating a disorder, comprising:

determining whether administration of a therapeutic agent or modality other than an anti-APRIL antibody molecule described herein reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in a subject in need thereof,

if the therapeutic agent or modality does not reduce, or is not likely to reduce, the level of a-g IgA by at least 40%, administering an anti-APRIL antibody molecule described herein to the subject at a dosage of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg.

91. A method of reducing the level of a-g IgA in subject, comprising:

administering an anti-APRIL antibody molecule described herein to a subject in a need thereof at a dose or dosage that reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in the subject,

wherein the dose or dosage is about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg;

thereby reducing the level of a-g IgA.

92. A method of selecting an anti-APRIL antibody molecule for treating a disorder, comprising:

determining whether administration of an anti-APRIL antibody molecule described herein at a dosage of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in a subject in need thereof,

thereby selecting the anti-APRIL antibody molecule.

93. A method of selecting a dose or dosage (e.g., dose and frequency) for an anti-APRIL antibody molecule for treating a disorder, comprising:

determining whether administration of an anti-APRIL antibody molecule described herein at a dose or dosage reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in a subject in need thereof,

wherein the dose or dosage is about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg;

thereby selecting the dose or dosage.

94. A method of selecting a subject for treating a disorder, comprising:

determining whether administration of an anti-APRIL antibody molecule described herein at a dosage of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg reduces, or is likely to reduce, the level of a-g IgA by at least 40% (e.g., by at least 40%, 50%, 60%, 70%, 75%, 80%, 85%, 90%, 95%, 96%, 97%, 98%, 99%, or 100%) in a subject in need thereof,

thereby selecting the subject,

optionally wherein if the antibody molecule does not reduce, or is not likely to reduce, the level of a-g IgA by at least 40%, administration of the antibody molecule is terminated, discontinued, or altered, or a different therapeutic agent or modality is administered.

95. The method of any of embodiments 79-94, wherein the a-g IgA comprises or is a-g IgA1.

96. The method of any of embodiments 79-95, wherein the level of a-g IgA is reduced by at least 40% (e.g., by at least 40%, 50%, 60%, 70%, 75%, 80%, 85%, 90%, 95%, 96%, 97%, 98%, 99%, or 100%) for a predetermined period, e.g., at least one, two, three, or four weeks, or at least one, two, or three months.

97. The method of any of embodiments 79-96, wherein the level of a-g IgA is reduced by at least 40% (e.g., by at least 40%, 50%, 60%, 70%, 75%, 80%, 85%, 90%, 95%, 96%, 97%, 98%, 99%, or 100%) about 4 weeks after the antibody molecule is administered.

98. The method of any of embodiments 79-97, wherein the level of a-g IgA is reduced by at least 40% (e.g., by at least 40%, 50%, 60%, 70%, 75%, 80%, 85%, 90%, 95%, 96%, 97%, 98%, 99%, or 100%) about 8 weeks after the antibody molecule is administered.

99. The method of any of embodiments 79-98, wherein the level of a-g IgA is reduced by at least 40% (e.g., by at least 40%, 50%, 60%, 70%, 75%, 80%, 85%, 90%, 95%, 96%, 97%, 98%, 99%, or

100%) about 12 weeks after the antibody molecule is administered. 100. The method of any of embodiments 79-99, wherein the level of a-g IgA is reduced by at least 40% (e.g., by at least 40%, 50%, 60%, 70%, 75%, 80%, 85%, 90%, 95%, 96%, 97%, 98%, 99%, or 100%) about 16 weeks after the antibody molecule is administered.

101. The method of any of embodiments 79-100, wherein the level of a-g IgA is reduced by at least 50%.

102. The method of any of embodiments 79-101, wherein the level of a-g IgA is reduced by at least 55%, 60%, 65%, 70%, 75%, 80%, 85%, 90%, or 95%.

103. The method of any of embodiments 79-102, wherein the antibody molecule is administered as a single dose, e.g., in a period of at least 1, 2, 3, 4, 5, 6, 7, 8, 9. 10, 11, 12, 13, 14, 15, 16, 17, or 18 months.

104. The method of any of embodiments 79-102, wherein the antibody molecule is administered as a repeated dose.

105. The method of any of embodiments 79-104, wherein the antibody molecule is administered subcutaneously.

106. The method of any of embodiments 79-104, wherein the antibody molecule is administered intravenously.

107. The method of any of embodiments 79-106, wherein the disorder is an APRIL-associated disorder.

108. The method of any of embodiments 79-107, wherein the disorder is associated with an aberrant level of total IgA.

109. The method of any of embodiments 79-108, wherein the disorder is a disorder associated with a-g IgA.

110. The method of any of embodiments 79-109, wherein the disorder is IgA nephropathy (IgAN).

111. The method of embodiment 110, wherein the IgAN is a familial IgAN.

112. The method of embodiment 110, wherein the IgAN is an adult IgAN.

113. The method of embodiment 110, wherein the IgAN is a post-transplant IgAN, a pediatric IgAN, or a crescentic IgAN.

114. The method of any of embodiments 79-108, wherein the disorder is a chronic kidney disease (CKD) or a disorder associated with CKD.

115. The method of embodiment 114, wherein the CKD is an advanced CKD, e.g., with an estimated glomerular filtration rate (eGFR) equal to or greater than about 30 or about 45.

116. The method of any of embodiments 79-108, wherein the disorder is Henoch-Schonlein purpura (HSP).

117. The method of any of embodiments 79-108, wherein the disorder is cutaneous vasculitis or IgA vasculitis.

118. The method of any of embodiments 79-108, wherein the disorder is IgA dermatitis, e.g., IgA bullous dermatosis.

119. The method of any of embodiments 79-108, wherein the disorder is Waldenstrom macroglobulinemia (WM).

120. The method of any of embodiments 79-108, wherein the disorder is lupus nephritis.

121. The method of any of embodiments 79-120, wherein the subject is a human.

122. The method of any of embodiments 79-121, wherein the subject has, or is identified as having, a level of a-g IgA that is at least 1, 1.5, 2, 2.5, 3.5, 4, 4.5, or 5-fold higher than the level of a-g IgA in a reference subject, e.g., a subject who does not have the disorder, e.g., a healthy or normal subject.

123. The method of any of embodiments 79-122, wherein the subject has, or is identified as having, a level of total IgA that is at least 1, 1.5, 2, 2.5, 3.5, 4, 4.5, or 5-fold higher than the level of total IgA in a reference subject, e.g., a subject who does not have the disorder, e.g., a healthy or normal subject.

124. The method of any of embodiments 79-123, wherein the subject has received, or is receiving, a different therapeutic agent or modality for treating the disorder.

125. The method of any of embodiments 79-123, wherein the subject has not received, or is not receiving, a different therapeutic agent or modality for treating the disorder.

126. The method of any of embodiments 79-125, wherein the subject has received, is receiving, or is going to receive, a vaccine, e.g., within 1, 2, 3, 4, 5, or 6 days, or 1, 2, 3, 4, 5, 6, 7, 8, 9, or 10 weeks, of administration of the antibody molecule.

127. The method of any of embodiments 79-125, wherein the subject is, or is identified as being, in need of receiving, a vaccine, e.g., within 1, 2, 3, 4, 5, or 6 days, or 1, 2, 3, 4, 5, 6, 7, 8, 9, or 10 weeks, of administration of the antibody molecule.

128. The method of embodiment 126 or 127, wherein the subject receives the vaccine before, concurrent with, or after administration of the antibody molecule.

129. The method of any of embodiments 126-128, wherein administration of the antibody molecule reduces the subject's ability to have an effective antigen-specific serum IgG and/or IgA response to the vaccine by no more than 5%, 10%, 15%, 20%, 25%, 30%, 35%, 40%, 45%, or 50%.

130. The method of any of embodiments 126-129, wherein administration of the antibody molecule does not reduce, or does not substantially reduce, the subject's ability to have an effective antigen-specific serum IgG and/or IgA response to the vaccine.

131. The method of any of embodiments 126-130, wherein the subject has or maintains an effective (e.g., protective) antigen-specific serum IgG and/or IgA response to the vaccine after administration of the antibody molecule.

132. The method of any of embodiments 126-131, wherein the vaccine comprises tetanus toxoid, diphtheria toxoid, or both (e.g., TENIVAC®).

133. The method of embodiment 132, wherein the subject has or maintains an effective (e.g., protective) level of tetanus and/or diphtheria anti-toxoid IgG (e.g., equal to or above 0.1 IU/mL in the blood), e.g., 1, 2, 3, 4, 5, 6, 7, 8, 9, 10, 11, 12, 13, 14, 15, 16, or more weeks after administration of the antibody molecule.

134. The method of any of embodiments 79-133, wherein the subject has, or is identified as having, a genomic susceptible locus of the disorder, e.g., IgA nephropathy.

135. The method of any of embodiments 79-134, further comprising determining whether the subject has a genomic susceptible locus of the disorder, e.g., IgA nephropathy.

136. The method of any of embodiments 79-135, wherein the antibody molecule comprises the HCDR1, HCDR2, HCDR3, LCDR1, LCDR2, and LCDR3 of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237.

137. The method of any of embodiments 79-136, wherein the antibody molecule comprises the VH and VL of any of antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237.

138. The method of any of embodiments 79-137, wherein the level of a-g IgA is determined in a sample from the subject.

139. The method of any of embodiments 79-138, further comprising determining the level of a-g IgA in a sample from the subject.

140. The method of any of embodiments 79-139, further comprising determining the level of total IgA in the sample.

141. The method of any of embodiments 79-140, further comprising determining the level of IgM and/or IgG in the sample.

142. The method of any of embodiments 79-141, further comprising obtaining a sample from the subject.

143. The method of embodiment 142, wherein the sample is a blood or serum sample.

144. The method of any of embodiments 79-143, further comprising administering a second therapeutic agent or modality to the subject.

145. The method of embodiment 144, wherein the second therapeutic agent or modality is a small molecule.

146. The method of embodiment 144, wherein the second therapeutic agent or modality is an antibody molecule.

147. The method or composition for use of any of the preceding embodiments, wherein the subject is administered the anti-APRIL antibody molecule at a concentration of about 100, 150, 175, 180, 190, 200, 210, 220, 225, 230, 240, 250, or 300 mg/mL.

148. The method or composition for use of any of the preceding embodiments, wherein the subject is administered the anti-APRIL antibody molecule at a concentration of about 200 mg/mL.

149. The method or composition for use of any of the preceding embodiments, wherein the subject is administered the anti-APRIL antibody molecule at a fixed dosage of about 200, 250, 300, 450, 400, 450, 500, 550, 600, 650, 700, 750, or 800 mg. 150. The method or composition for use of any of the preceding embodiments, wherein the subject is administered the anti-APRIL antibody molecule at a fixed dosage of about 200 mg (e.g., at a volume of about 1 mL).

151. The method or composition for use of any of the preceding embodiments, wherein the subject is administered the anti-APRIL antibody molecule at a fixed dosage of about 400 mg (e.g., at a total volume of about 2 mL, e.g., as two administrations of 1 mL volumes or as one administration of a 2 mL volume).

152. The method or composition for use of any of the preceding embodiments, wherein the subject is administered the anti-APRIL antibody molecule at a fixed dosage of at least 200 mg.

153. The method or composition for use of any of the preceding embodiments, wherein the subject is administered the anti-APRIL antibody molecule at a fixed dosage of 800 mg or less.

154. The method or composition for use of any of the preceding embodiments, wherein the subject is administered the anti-APRIL antibody molecule at a fixed dosage of about 600 mg (e.g., at a total volume of about 3 mL, e.g., as one administration of a 2 mL volume and one administration of a 1 mL volume).

155. The method or composition for use of any of the preceding embodiments, wherein the subject is administered a single dose of the anti-APRIL antibody molecule.

156. The method or composition for use of any of the preceding embodiments, wherein the subject is administered one or more additional dosages of the anti-APRIL antibody molecules (e.g., 24 hours, 48 hours, 72 hours, 4 days, 5 days, 6 days, 1 week, 2 weeks, 3 weeks, 4 weeks, 1 month, 2 months, 3 months, 4 months, 5 months, or 6 months after the first administration).

157. The method or composition for use of any of the preceding embodiments, wherein the subject is administered the anti-APRIL antibody molecule subcutaneously.

158. The method or composition for use of any of the preceding embodiments, wherein the subject is administered the anti-APRIL antibody molecule intravenously.

159. The method or composition for use of any of the preceding embodiments, wherein the anti-APRIL antibody molecule is administered as a liquid.

160. An anti-APRIL antibody molecule, or a pharmaceutical composition comprising the anti-APRIL antibody molecule, for use in a method of treating a disorder in a human subject,

wherein the antibody molecule is administered at a dose of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg; wherein the administration reduces the level of aberrantly glycosylated IgA (a-g IgA) by at least 40%, 50%, 60%, 70%, 75%, 80%, 85%, 90%, 95%, 96%, 97%, 98%, 99%, or 100% in the subject; and

optionally wherein the antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3),

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16,

optionally wherein the disorder is IgA nephropathy.

161. An anti-APRIL antibody molecule, or a pharmaceutical composition comprising the anti-APRIL antibody molecule, for use in a method of reducing the level of a-g IgA in a human subject,

wherein the antibody molecule is administered at a dose of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg;

wherein the administration reduces the level of a-g IgA by at least 40%, 50%, 60%, 70%, 75%, 80%, 85%, 90%, 95%, 96%, 97%, 98%, 99%, or 100% in the subject; and

optionally wherein the antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3),

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16,

optionally wherein the subject has, or is at risk of having, a disorder, e.g., IgA nephropathy.

162. An anti-APRIL antibody molecule, or a pharmaceutical composition comprising the anti-APRIL antibody molecule, for use in a method of treating a disorder in a human subject,

wherein the method comprises selecting a dose or dosage for the antibody molecule;

wherein administration of the antibody molecule at the selected dose or dosage reduces, or is likely to reduce, the level of a-g IgA by at least 40%, 50%, 60%, 70%, 75%, 80%, 85%, 90%, 95%, 96%, 97%, 98%, 99%, or 100% in the subject; and

optionally wherein the antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3),

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16,

optionally wherein the antibody molecule is administered at a dose of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg,

optionally wherein the disorder is IgA nephropathy.

163. An anti-APRIL antibody molecule, or a pharmaceutical composition comprising the anti-APRIL antibody molecule, for use in a method of treating a disorder in a human subject,

wherein the method comprises responsive to a determination that administration of the antibody molecule reduces, or is likely to reduce, the level of a-g IgA by at least 40%, 50%, 60%, 70%, 75%, 80%, 85%, 90%, 95%, 96%, 97%, 98%, 99%, or 100% in the subject, administering to the subject the antibody molecule at a dose of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg; and

optionally wherein the antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3),

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16,

optionally wherein the disorder is IgA nephropathy.

164. An anti-APRIL antibody molecule, or a pharmaceutical composition comprising the anti-APRIL antibody molecule, for use in a method of treating a disorder in a human subject,

wherein the method comprises determining whether administration of an anti-APRIL antibody molecule reduces, or is likely to reduce, the level of a-g IgA by at least 40%, 50%, 60%, 70%, 75%, 80%, 85%, 90%, 95%, 96%, 97%, 98%, 99%, or 100% in the subject,

if the antibody molecule reduces, or is likely to reduce, the level of a-g IgA by at least 40%, 50%, 60%, 70%, 75%, 80%, 85%, 90%, 95%, 96%, 97%, 98%, 99%, or 100%, administration of the antibody molecule at a dosage of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg is initiated, continued, or maintained; and

optionally wherein the antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3),

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16,

optionally wherein the antibody molecule is administered at a dose of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg,

optionally wherein the disorder is IgA nephropathy,

optionally wherein if the antibody molecule does not reduce, or is not likely to reduce, the level of a-g IgA by at least 40%, 50%, 60%, 70%, 75%, 80%, 85%, 90%, 95%, 96%, 97%, 98%, 99%, or 100%, administration of the antibody molecule is terminated, discontinued, or altered, and/or a different therapeutic agent or modality is administered.

165. An anti-APRIL antibody molecule, or a pharmaceutical composition comprising the anti-APRIL antibody molecule, for use in a method of treating a disorder in a human subject,

wherein the method comprises determining whether administration of a therapeutic agent or modality other than the antibody molecule reduces, or is likely to reduce, the level of a-g IgA by at least 40%, 50%, 60%, 70%, 75%, 80%, 85%, 90%, 95%, 96%, 97%, 98%, 99%, or 100% in a subject in need thereof,

if the therapeutic agent or modality does not reduce, or is not likely to reduce, the level of a-g IgA by at least 40%, 50%, 60%, 70%, 75%, 80%, 85%, 90%, 95%, 96%, 97%, 98%, 99%, or 100%, administering the antibody molecule to the subject at a dose of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg; and

optionally wherein the antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3),

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16,

optionally wherein the disorder is IgA nephropathy.

166. An anti-APRIL antibody molecule, or a pharmaceutical composition comprising the anti-APRIL antibody molecule, for use in a method of treating a disorder in a human subject,

wherein the antibody molecule is administered at a dose of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg; and

wherein the subject has received, or is going to receive, a vaccine within 1, 2, 3, 4, 5, 6, 7, 8, 9, 10, 11, 12, 13, 14, or 15 weeks of administration of the antibody molecule, optionally wherein the vaccine comprises tetanus toxoid, diphtheria toxoid, or both (e.g., TENIVAC®),

optionally wherein the antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3),

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16,

optionally wherein the disorder is IgA nephropathy,

optionally wherein administration of the antibody molecule at the selected dose or dosage reduces, or is likely to reduce, the level of a-g IgA by at least 40%, 50%, 60%, 70%, 75%, 80%, 85%, 90%, 95%, 96%, 97%, 98%, 99%, or 100% in the subject.

167. A method of treating a disorder, comprising:

administering an anti-APRIL antibody molecule to a human subject in need thereof at a dose of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg;

wherein the administration reduces the level of aberrantly glycosylated IgA (a-g IgA) by at least 40%, 50%, 60%, 70%, 75%, 80%, 85%, 90%, 95%, 96%, 97%, 98%, 99%, or 100% in the subject; and wherein the antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3),

optionally wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16,

optionally wherein the disorder is IgA nephropathy,

thereby treating the disorder.

168. A method of reducing the level of a-g IgA, comprising:

administering an anti-APRIL antibody molecule to a human subject in need thereof,

wherein the antibody molecule is administered at a dose of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg;

wherein the administration reduces the level of a-g IgA by at least 40%, 50%, 60%, 70%, 75%, 80%, 85%, 90%, 95%, 96%, 97%, 98%, 99%, or 100% in the subject; and

optionally wherein the antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3),

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16,

optionally wherein the subject has, or is at risk of having, a disorder, e.g., IgA nephropathy,

thereby reducing the level of a-g IgA.

169. A method of treating a disorder, comprising:

selecting a dose or dosage for an anti-APRIL antibody molecule;

wherein administration of the antibody molecule at the selected dose or dosage reduces, or is likely to reduce, the level of a-g IgA by at least 40%, 50%, 60%, 70%, 75%, 80%, 85%, 90%, 95%, 96%, 97%, 98%, 99%, or 100% in the subject; and

optionally wherein the antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3),

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16,

optionally wherein the antibody molecule is administered at a dose of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg,

optionally wherein the subject has, or is at risk of having, IgA nephropathy,

thereby threating the disorder.

170. A method of treating a disorder, comprising:

responsive to a determination that administration of the antibody molecule reduces, or is likely to reduce, the level of a-g IgA by at least 40%, 50%, 60%, 70%, 75%, 80%, 85%, 90%, 95%, 96%, 97%, 98%, 99%, or 100% in the subject, administering to a human subject in need thereof an anti-APRIL antibody molecule at a dose of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg; and

optionally wherein the antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3),

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16,

optionally wherein the disorder is IgA nephropathy,

thereby treating the disorder.

171. A method of treating a disorder, comprising:

determining whether administration of an anti-APRIL antibody molecule reduces, or is likely to reduce, the level of a-g IgA by at least 40%, 50%, 60%, 70%, 75%, 80%, 85%, 90%, 95%, 96%, 97%, 98%, 99%, or 100% in the subject,

if the antibody molecule reduces, or is likely to reduce, the level of a-g IgA by at least 40%, 50%, 60%, 70%, 75%, 80%, 85%, 90%, 95%, 96%, 97%, 98%, 99%, or 100%, administration of the antibody molecule at a dosage of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg is initiated, continued, or maintained; and

optionally wherein the antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3),

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16,

optionally wherein the antibody molecule is administered at a dose of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg,

optionally wherein if the antibody molecule does not reduce, or is not likely to reduce, the level of a-g IgA by at least 40%, 50%, 60%, 70%, 75%, 80%, 85%, 90%, 95%, 96%, 97%, 98%, 99%, or 100%, administration of the antibody molecule is terminated, discontinued, or altered, and/or a different therapeutic agent or modality is administered,

optionally wherein the disorder is IgA nephropathy,

thereby treating the disorder.

172. A method of treating a disorder, comprising:

determining whether administration of a therapeutic agent or modality other than an anti-APRIL antibody molecule reduces, or is likely to reduce, the level of a-g IgA by at least 40%, 50%, 60%, 70%, 75%, 80%, 85%, 90%, 95%, 96%, 97%, 98%, 99%, or 100% in a subject in need thereof,

if the therapeutic agent or modality does not reduce, or is not likely to reduce, the level of a-g IgA by at least 40%, 50%, 60%, 70%, 75%, 80%, 85%, 90%, 95%, 96%, 97%, 98%, 99%, or 100%, administering the antibody molecule to a human subject at a dose of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg; and

optionally wherein the antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3),

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16,

optionally wherein the disorder is IgA nephropathy,

thereby treating the disorder.

173. A method of treating a disorder, comprising:

administering an anti-APRIL antibody molecule to a human subject in need thereof at a dose of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg; and

wherein the subject has received, or is going to receive, a vaccine within 1, 2, 3, 4, 5, 6, 7, 8, 9, 10, 11, 12, 13, 14, or 15 weeks of administration of the antibody molecule, optionally wherein the vaccine comprises tetanus toxoid, diphtheria toxoid, or both (e.g., TENIVAC®),

optionally wherein the antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3),

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16,

optionally wherein the disorder is IgA nephropathy,

optionally wherein administration of the antibody molecule at the selected dose or dosage reduces, or is likely to reduce, the level of a-g IgA by at least 40%, 50%, 60%, 70%, 75%, 80%, 85%, 90%, 95%, 96%, 97%, 98%, 99%, or 100% in the subject,

thereby treating the disorder.

174. A method of selecting an anti-APRIL antibody molecule for treating a disorder, comprising: determining whether administration of the antibody molecule at a dose or dosage reduces, or is likely to reduce, the level of a-g IgA by at least 40%, 50%, 60%, 70%, 75%, 80%, 85%, 90%, 95%, 96%, 97%, 98%, 99%, or 100% in a human subject in need thereof,

wherein the dose is about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg,

optionally wherein the antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3),

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16,

optionally wherein the disorder is IgA nephropathy,

thereby selecting the antibody molecule.

175. A method of selecting a dose or dosage for an anti-APRIL antibody molecule for treating a disorder, comprising:

determining whether administration of the antibody molecule at a dose or dosage reduces, or is likely to reduce, the level of a-g IgA by at least 40%, 50%, 60%, 70%, 75%, 80%, 85%, 90%, 95%, 96%, 97%, 98%, 99%, or 100% in a human subject in need thereof,

optionally wherein the dose is about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg,

optionally wherein the antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3),

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16,

optionally wherein the disorder is IgA nephropathy,

thereby selecting the dose or dosage.

176. A method of selecting a human subject for treating a disorder, comprising:

determining whether administration of an anti-APRIL antibody molecule at a dosage of about 0.5 mg/kg, 2.0 mg/kg, 6 mg/kg, 9 mg/kg, 9.1 mg/kg, 12 mg/kg, or at a fixed dose of about 200 mg, 400 mg, 600 mg, or 800 mg reduces, or is likely to reduce, the level of a-g IgA by at least 40%, 50%, 60%, 70%, 75%, 80%, 85%, 90%, 95%, 96%, 97%, 98%, 99%, or 100% in the subject,

optionally wherein the antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3),

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16,

optionally wherein the disorder is IgA nephropathy,

thereby selecting the subject,

177. The antibody molecule, pharmaceutical composition, method for use of any of embodiments 160-176, wherein the a-g IgA comprises or is a-g IgA1.

178. The antibody molecule, pharmaceutical composition, method for use of any of embodiments 160-177, wherein the level of a-g IgA is reduced by at least 40%, 50%, 60%, 70%, 75%, 80%, 85%, 90%, 95%, 96%, 97%, 98%, 99%, or 100% for a predetermined period, e.g., at least one, two, three, or four weeks, or at least one, two, three, four, five, six, seven, eight, nine, ten, eleven, or twelve months.

179. The antibody molecule, pharmaceutical composition, method for use of any of embodiments 160-178, wherein the level of a-g IgA is reduced by at least 40%, 50%, 60%, 70%, 75%, 80%, 85%, 90%, 95%, 96%, 97%, 98%, 99%, or 100% about 4 weeks after the antibody molecule is administered.

180. The antibody molecule, pharmaceutical composition, method for use of any of embodiments 160-179, wherein the level of a-g IgA is reduced by at least 40%, 50%, 60%, 70%, 75%, 80%, 85%, 90%, 95%, 96%, 97%, 98%, 99%, or 100% about 8 weeks after the antibody molecule is administered.

181. The antibody molecule, pharmaceutical composition, method for use of any of embodiments 160-180, wherein the level of a-g IgA is reduced by at least 40%, 50%, 60%, 70%, 75%, 80%, 85%, 90%, 95%, 96%, 97%, 98%, 99%, or 100% about 12 weeks after the antibody molecule is administered.

182. The antibody molecule, pharmaceutical composition, method for use of any of embodiments 160-181, wherein the level of a-g IgA is reduced by at least 40%, 50%, 60%, 70%, 75%, 80%, 85%, 90%, 95%, 96%, 97%, 98%, 99%, or 100% about 16 weeks after the antibody molecule is administered.

183. The antibody molecule, pharmaceutical composition, method for use of any of embodiments 160-182, wherein the level of a-g IgA is reduced by at least 50%.

184. The antibody molecule, pharmaceutical composition, method for use of any of embodiments 160-183, wherein the level of a-g IgA is reduced by at least 55%, 60%, 65%, 70%, 75%, 80%, 85%, 90%, or 95%.

185. The antibody molecule, pharmaceutical composition, method for use of any of embodiments 160-184, e.g., in a period of at least 1, 2, 3, 4, 5, 6, 7, 8, 9. 10, 11, 12, 13, 14, 15, 16, 17, or 18 months.

186. The antibody molecule, pharmaceutical composition, method for use of any of embodiments 160-185, wherein the antibody molecule is administered as a repeated dose, e.g., in a period of at least 3, 6, 9. 12, 15, 18, 24, 30, or 36 months, optionally wherein the subject is administered one or more additional dosages of the anti-APRIL antibody molecules (e.g., 24 hours, 48 hours, 72 hours, 4 days, 5 days, 6 days, 1 week, 2 weeks, 3 weeks, 4 weeks, 1 month, 2 months, 3 months, 4 months, 5 months, or 6 months after the first administration).

187. The antibody molecule, pharmaceutical composition, method for use of any of embodiments 160-186, wherein the antibody molecule is administered subcutaneously.

188. The antibody molecule, pharmaceutical composition, method for use of any of embodiments 160-186, wherein the antibody molecule is administered intravenously.

189. The antibody molecule, pharmaceutical composition, method for use of any of embodiments 160-188, wherein the disorder an APRIL-associated disorder.

190. The antibody molecule, pharmaceutical composition, method for use of any of embodiments 160-189, wherein the disorder is associated with an aberrant level of total IgA.

191. The antibody molecule, pharmaceutical composition, method for use of any of embodiments 160-190, wherein the disorder is a disorder associated with a-g IgA.

192. The antibody molecule, pharmaceutical composition, method for use of any of embodiments 160-191, wherein the disorder is IgA nephropathy (IgAN).

193. The antibody molecule, pharmaceutical composition, method for use of embodiment 192, wherein the IgAN is a familial IgAN.

194. The antibody molecule, pharmaceutical composition, method for use of embodiment 192, wherein the IgAN is an adult IgAN.

195. The antibody molecule, pharmaceutical composition, method for use of embodiment 192, wherein the IgAN is a post-transplant IgAN, a pediatric IgAN, or a crescentic IgAN.

196. The antibody molecule, pharmaceutical composition, method for use of any of embodiments 160-191, wherein the disorder is a chronic kidney disease (CKD) or a disorder associated with CKD.

197. The antibody molecule, pharmaceutical composition, method for use of embodiment 196, wherein the CKD is an advanced CKD, e.g., with an estimated glomerular filtration rate (eGFR) equal to or greater than about 30 or about 45.

198. The antibody molecule, pharmaceutical composition, method for use of any of embodiments 160-191, wherein the disorder is Henoch-Schonlein purpura (HSP).

199. The antibody molecule, pharmaceutical composition, method for use of any of embodiments 160-191, wherein the disorder is cutaneous vasculitis or IgA vasculitis.

200. The antibody molecule, pharmaceutical composition, method for use of any of embodiments 160-191, wherein the disorder is IgA dermatitis, e.g., IgA bullous dermatosis.

201. The antibody molecule, pharmaceutical composition, method for use of any of embodiments 160-191, wherein the disorder is Waldenstrom macroglobulinemia (WM).

202. The antibody molecule, pharmaceutical composition, method for use of any of embodiments 160-191, wherein the disorder is lupus nephritis.

203. The antibody molecule, pharmaceutical composition, method for use of any of embodiments 160-202, wherein the subject is a human patient.

204. The antibody molecule, pharmaceutical composition, method for use of any of embodiments 160-203, wherein the subject has, or is identified as having, a level of a-g IgA that is at least 1, 1.5, 2, 2.5, 3.5, 4, 4.5, or 5-fold higher than the level of a-g IgA in a reference subject, e.g., a subject who does not have the disorder, e.g., a healthy or normal subject.

205. The antibody molecule, pharmaceutical composition, method for use of any of embodiments 160-204, wherein the subject has, or is identified as having, a level of total IgA that is at least 1, 1.5, 2, 2.5, 3.5, 4, 4.5, or 5-fold higher than the level of total IgA in a reference subject, e.g., a subject who does not have the disorder, e.g., a healthy or normal subject.

206. The antibody molecule, pharmaceutical composition, method for use of any of embodiments 160-205, wherein the subject has received, or is receiving, a different therapeutic agent or modality for treating the disorder.

207. The antibody molecule, pharmaceutical composition, method for use of any of embodiments 160-206, wherein the subject has not received, or is not receiving, a different therapeutic agent or modality for treating the disorder.

208. The antibody molecule, pharmaceutical composition, method for use of any of embodiments 160-207, wherein the subject has received, is receiving, or is going to receive, a vaccine, e.g., within 1, 2, 3, 4, 5, or 6 days, or 1, 2, 3, 4, 5, 6, 7, 8, 9, or 10 weeks, of administration of the antibody molecule.

209. The antibody molecule, pharmaceutical composition, method for use of any of embodiments 160-208, wherein the subject is, or is identified as being, in need of receiving, a vaccine, e.g., within 1, 2, 3, 4, 5, or 6 days, or 1, 2, 3, 4, 5, 6, 7, 8, 9, or 10 weeks, of administration of the antibody molecule.

210. The antibody molecule, pharmaceutical composition, method for use of any of embodiments 208 or 209, wherein the subject receives the vaccine before, concurrent with, or after administration of the antibody molecule.

211. The antibody molecule, pharmaceutical composition, method for use of any of embodiments 160-210, wherein administration of the antibody molecule reduces the subject's ability to have an effective antigen-specific serum IgG and/or IgA response to the vaccine by no more than 5%, 10%, 15%, 20%, 25%, 30%, 35%, 40%, 45%, or 50%.

212. The antibody molecule, pharmaceutical composition, method for use of any of embodiments 160-211, wherein administration of the antibody molecule does not reduce, or does not substantially reduce, the subject's ability to have an effective antigen-specific serum IgG and/or IgA response to the vaccine.

213. The antibody molecule, pharmaceutical composition, method for use of any of embodiments 160-212, wherein the subject has or maintains an effective (e.g., protective) antigen-specific serum IgG and/or IgA response to the vaccine after administration of the antibody molecule.

214. The antibody molecule, pharmaceutical composition, method for use of any of embodiments 160-213, wherein the vaccine comprises tetanus toxoid, diphtheria toxoid, or both (e.g., TENIVAC®).

215. The antibody molecule, pharmaceutical composition, method for use of embodiment 214, wherein the subject has or maintains an effective (e.g., protective) level of tetanus and/or diphtheria anti-toxoid IgG (e.g., equal to or above 0.1 IU/mL in the blood), e.g., 1, 2, 3, 4, 5, 6, 7, 8, 9, 10, 11, 12, 13, 14, 15, 16, or more weeks after administration of the antibody molecule.

216. The antibody molecule, pharmaceutical composition, method for use of any of embodiments 160-215, wherein the subject has, or is identified as having, a genomic susceptible locus of the disorder, e.g., IgA nephropathy.

217. The antibody molecule, pharmaceutical composition, method for use of any of embodiments 160-216, further comprising determining whether the subject has a genomic susceptible locus of the disorder, e.g., IgA nephropathy.

218. The antibody molecule, pharmaceutical composition, method for use of any of embodiments 160-217, wherein the antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3),

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16.

219. The antibody molecule, pharmaceutical composition, method for use of any of embodiments 160-218, wherein the antibody molecule comprises a VH comprising the amino acid sequence of SEQ ID NO: 296, and a VL comprising the amino acid sequence of SEQ ID NO: 286,

optionally wherein the antibody molecule is an IgG2.

220. The antibody molecule, pharmaceutical composition, method for use of any of embodiments 160-219, wherein the level of a-g IgA is determined in a sample from the subject.

221. The antibody molecule, pharmaceutical composition, method for use of any of embodiments 160-220, further comprising determining the level of a-g IgA in a sample from the subject.

222. The antibody molecule, pharmaceutical composition, method for use of any of embodiments 160-221, further comprising determining the level of total IgA in the sample.

223. The antibody molecule, pharmaceutical composition, method for use of any of embodiments 160-222, further comprising determining the level of IgM and/or IgG in the sample.

224. The antibody molecule, pharmaceutical composition, method for use of any of embodiments 160-223, further comprising obtaining a sample from the subject.

225. The antibody molecule, pharmaceutical composition, method for use of embodiment 224, wherein the sample is a blood or serum sample.

226. The antibody molecule, pharmaceutical composition, method for use of any of embodiments 160-225, further comprising administering a second therapeutic agent or modality to the subject.

227. The antibody molecule, pharmaceutical composition, method for use of embodiment 226, wherein the second therapeutic agent or modality is a small molecule.

228. The antibody molecule, pharmaceutical composition, method for use of embodiment 227, wherein the second therapeutic agent or modality is an antibody molecule.

229. The antibody molecule, pharmaceutical composition, method for use of any of embodiments 160-228, wherein the subject is administered the anti-APRIL antibody molecule at a concentration of about 100, 150, 175, 180, 190, 200, 210, 220, 225, 230, 240, 250, or 300 mg/mL.

230. The antibody molecule, pharmaceutical composition, method for use of any of embodiments 160-229, wherein the subject is administered the anti-APRIL antibody molecule at a concentration of about 200 mg/mL.

231. The antibody molecule, pharmaceutical composition, method for use of any of embodiments 160-230, wherein the subject is administered the anti-APRIL antibody molecule at a fixed dose of about 200, 250, 300, 450, 400, 450, 500, 550, 600, 650, 700, 750, or 800 mg.

232. The antibody molecule, pharmaceutical composition, method for use of any of embodiments 160-231, wherein the subject is administered the anti-APRIL antibody molecule at a fixed dose of about 200 mg (e.g., at a volume of about 1 mL).

233. The antibody molecule, pharmaceutical composition, method for use of any of embodiments 160-232, wherein the subject is administered the anti-APRIL antibody molecule at a fixed dose of about 400 mg (e.g., at a total volume of about 2 mL, e.g., as two administrations of 1 mL volumes or as one administration of a 2 mL volume).

234. The antibody molecule, pharmaceutical composition, method for use of any of embodiments 160-233, wherein the subject is administered the anti-APRIL antibody molecule at a fixed dose of about 600 mg (e.g., at a total volume of about 3 mL, e.g., as one administration of a 2 mL volume and one administration of a 1 mL volume).

235. The antibody molecule, pharmaceutical composition for use, or method of any of the preceding embodiments, wherein the subject is administered the anti-APRIL antibody molecule at a fixed dosage of at least 200 mg.

236. The antibody molecule, pharmaceutical composition for use, or method of any of the preceding embodiments, wherein the subject is administered the anti-APRIL antibody molecule at a fixed dosage of 800 mg or less.

237. The antibody molecule, pharmaceutical composition for use, or method of any of the preceding embodiments, wherein the anti-APRIL antibody molecule is administered as a liquid composition.

238. A method of improving kidney function, the method comprising administering to a subject in need thereof an anti-APRIL antibody molecule, thereby improving kidney function.

239. The method of embodiment 238, wherein the method reverses or prevents progression of decreased kidney function in the subject.

240. The method of any of embodiments 238-239, wherein the improved kidney function comprises kidney regeneration in the subject.

241. The method of any of embodiments 238-240, wherein the improved kidney function comprises increased estimated glomerular filtrate rate (eGFR) in the kidney of the subject.

242. The method of any of embodiments 238-241, wherein the improved kidney function comprises reduced proteinuria in the kidney of the subject.

243. The method of any of embodiments 238-242, wherein the method improves kidney function in the subject within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after administration (e.g., after first, second, or third administration) of the antibody molecule.

244. The method of any of embodiments 238-243, wherein the method improves kidney function in the subject after 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 administrations of the antibody molecule.

245. The method of any of embodiments 238-244, wherein the method improves kidney function for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months.

246. A method of preserving (e.g., maintaining or increasing) estimated glomerular filtrate rate (eGFR) in the kidney, the method comprising administering to a subject in need thereof an anti-APRIL antibody molecule comprising the HCDR1, HCDR2, HCDR3, LCDR1, LCDR2, and LCDR3 of antibody 2419-1406 or the VH and VL of antibody 2419-1406, thereby preserving (e.g., maintaining or increasing) eGFR in the kidney.

247. A method of reducing proteinuria in the kidney, the method comprising administering to a subject in need thereof an anti-APRIL antibody molecule, thereby reducing proteinuria.

248. The method of embodiment 247, wherein the reduction in proteinuria is determined by measuring urine protein/creatinine ratio (uPCR), e.g., as described in Example 8.

249. The method of any of embodiments 247-248, wherein the method reduces proteinuria in the subject within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after administration (e.g., after first, second, or third administration) of the antibody molecule.

250. The method of embodiment 249, wherein the method reduces proteinuria in the subject within about 3 months after administration.

251. The method of embodiment 249, wherein the method reduces proteinuria in the subject within about 6 months after administration.

252. The method of embodiment 249, wherein the method reduces proteinuria in the subject within about 1 year after administration.

253. The method of embodiment 249, wherein the method reduces proteinuria in the subject within about 2 years after administration.

254. The method of embodiment 249, wherein the method reduces proteinuria in the subject within about 3 years after administration.

255. The method of embodiment 249, wherein the method reduces proteinuria in the subject within about 4 years after administration.

256. The method of any of embodiments 247-255, wherein the method reduces proteinuria in the subject after 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 administrations of the antibody molecule.

257. The method of any of embodiments 247-256, wherein the method reduces proteinuria for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months.

258. A method of inducing kidney recovery, the method comprising administering to a subject in need thereof an anti-APRIL antibody molecule,

thereby inducing kidney recovery.

259. A method of inducing kidney regeneration, the method comprising administering to a subject in need thereof an anti-APRIL antibody molecule,

thereby inducing kidney regeneration.

260. A method of reducing autoantibody response, the method comprising administering to a subject in need thereof an anti-APRIL antibody molecule,

thereby reducing autoantibody response.

261. The method of any of the preceding embodiments, wherein the antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3),

wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or

wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16.

262. The method of any of the preceding embodiments, wherein the antibody molecule comprises a VH comprising the amino acid sequence of SEQ ID NO: 296, an amino acid sequence having at least 85%, 90%, 95%, 96%, 97%, 98%, or 99% identity thereto, or an amino acid sequence differing by no more than 1, 2, 3, 4, 5, 6, 7, 8, 9, or 10 amino acids therefrom.

263. The method of any of the preceding embodiments, wherein the antibody molecule comprises a VL comprising the amino acid sequence of SEQ ID NO: 286, or an amino acid sequence having at least 85%, 90%, 95%, 96%, 97%, 98%, or 99% identity thereto, or an amino acid sequence differing by no more than 1, 2, 3, 4, 5, 6, 7, 8, 9, or 10 amino acids therefrom.

264. The method of any of the preceding embodiments, wherein the antibody molecule comprises a VH comprising the amino acid sequence of SEQ ID NO: 296, and a VL comprising the amino acid sequence of SEQ ID NO: 286.

265. The method of any of the preceding embodiments, wherein the antibody molecule comprises a heavy chain constant region of IgG2 and a light chain constant region of kappa.

266. The method of any of the preceding embodiments, wherein the antibody molecule is sibeprenlimab.

267. The method of any of the preceding embodiments, wherein the antibody molecule is administered no more than once a month.

268. The method of any of the preceding embodiments, wherein the antibody molecule is administered at a dose of 2 mg/kg, 4 mg/kg, or 8 mg/kg.

269. The method of any of the preceding embodiments, wherein the antibody molecule is administered once a month, once every two months, or once every three months.

270. The method of any of the preceding embodiments, wherein the antibody molecule is administered to repeatedly, e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 times.

271. The method of any of the preceding embodiments, wherein the antibody molecule is administered once a month for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months.

272. The method of any of the preceding embodiments, wherein the antibody molecule is administered intravenously or subcutaneously.

273. The method of any of the preceding embodiments, wherein the antibody molecule is administered at a dose of 4 mg/kg, e.g., once a month, and the administration results in an increase in eGFR within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after the administration (e.g., after the first, second, or third administration).

274. The method of any of the preceding embodiments, wherein the antibody molecule is administered at a dose of 8 mg/kg, e.g., once a month, and the administration results in an increase in eGFR within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after the administration (e.g., after the first, second, or third administration).

275. The method of embodiment 273 or 274, wherein eGFR is increased by at least 10%, 20%, 30%, 40%, 50%, 60%, 70%, 75%, 80%, 85%, 90%, 95%, 96%, 97%, 98%, or 99%, e.g., within about 1, 2,3,4,5,6,7, 8,9, 10, 11, 12, 13, 14, 15, 16, 17, 18, 19, 20, 21, 22, 23, 24, 25, 26, 27, 28, 29, 30, 31, 32, 33, 34, 35, or 36 months after the administration (e.g., after the first, second, or third administration).

276. The method of any of the preceding embodiments, wherein administration of the antibody molecule results in a decrease in serum IgA levels (e.g., compared to prior to the administration).

277. The method of embodiment 276, wherein the decrease in serum IgA levels is maintained for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months.

278. The method of embodiment 276 or 277, wherein the serum IgA levels are decreased by at least 40%, 50%, 60%, 70%, 75%, 80%, 85%, 90%, 95%, 96%, 97%, 98%, or 99%, e.g., within 1, 2, 3, 4, 5,6,7, 8,9, 10, 11, 12, 13, 14, 15, 16, 17, 18, 19, 20, 21, 22, 23, 24, 25, 26, 27, 28, 29, 30, 31, 32, 33, 34, 35, or 36 months after the administration (e.g., after the first, second, or third administration).

279. The method of any of embodiments 276-278, wherein the serum IgA levels are decreased by at least 50, 100, 110, 120, 130, 140, 150, 160, 170, 180, 190, 200, 210, 220, 230, 240, or 250 mg/dL, e.g., within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after the administration (e.g., after the first, second, or third administration).

280. The method of any of the preceding embodiments, wherein administration of the anti-APRIL antibody molecule results in a decrease in serum a-g-IgA levels (e.g., compared to prior to the administration).

281. The method of embodiment 280, wherein the decrease in serum a-g-IgA levels is maintained for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months.

282. The method of embodiment 280 or 281, wherein the serum a-g-IgA levels are decreased by at least 40%, 50%, 60%, 70%, 75%, 80%, 85%, 90%, 95%, 96%, 97%, 98%, or 99%, e.g., within 1, 2, 3, 4,5,6,7, 8,9, 10, 11, 12, 13, 14, 15, 16, 17, 18, 19, 20, 21, 22, 23, 24, 25, 26, 27, 28, 29, 30, 31, 32, 33, 34, 35, or 36 months after the administration (e.g., after the first, second, or third administration).

283. The method of any of embodiments 280-282, wherein the a-g-IgA comprises or is a-g-IgA1.

284. The method of any of the preceding embodiments, wherein administration of the antibody molecule results in a decrease in serum IgG levels (e.g., compared to prior to the administration).

285. The method of embodiment 284, wherein the decrease in serum IgG levels is maintained for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months.

286. The method of embodiment 284 or 285, wherein the serum IgG levels are decreased by at least 20%, 25%, 30%, 35%, 40%, 45%, 50%, 55%, 60%, 70%, 75%, 80%, 85%, or 90%, e.g., within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after the administration (e.g., after the first, second, or third administration).

287. The method of any of embodiments 284-286, wherein the serum IgG levels are decreased by at least 100, 150, 200, 250, 300, 350, 400, 450, 500, 550, 600, 650, 700, 750, or 800 mg/dL, e.g., within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after the administration (e.g., after the first, second, or third administration).

288. The method of any of the preceding embodiments, wherein administration of the antibody molecule results in a decrease in serum IgM levels (e.g., compared to prior to the administration).

289. The method of embodiment 288, wherein the decrease in serum IgM levels is maintained for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months.

290. The method of embodiment 288 or 289, wherein the serum IgM levels are decreased by at least 40%, 50%, 60%, 70%, 75%, 80%, 85%, 90%, 95%, 96%, 97%, 98%, or 99%, e.g., within 1, 2, 3, 4, 5,6,7, 8,9, 10, 11, 12, 13, 14, 15, 16, 17, 18, 19, 20, 21, 22, 23, 24, 25, 26, 27, 28, 29, 30, 31, 32, 33, 34, 35, or 36 months after the administration (e.g., after the first, second, or third administration).

291. The method of any of embodiments 288-290, wherein the serum IgM levels are decreased by at least 10, 20, 25, 30, 35, 40, 45, 50, 55, 60, 65, 70, 75, 80, 85, 90, or 95 mg/dL, e.g., within 1, 2, 3, 4, 5,6,7, 8,9, 10, 11, 12, 13, 14, 15, 16, 17, 18, 19, 20, 21, 22, 23, 24, 25, 26, 27, 28, 29, 30, 31, 32, 33, 34, 35, or 36 months after the administration (e.g., after the first, second, or third administration).

292. The method of any of the preceding embodiments, wherein administration of the anti-APRIL antibody molecule results in a decrease in uPCR (e.g., compared to prior to the administration).

293. The method of embodiment 288, wherein the decrease in uPCR is maintained for at least 1, 2,3,4,5,6,7, 8,9, 10, 11, 12, 13, 14, 15, 16, 17, 18, 19, 20, 21, 22, 23, 24, 25, 26, 27, 28, 29, 30, 31, 32, 33, 34, 35, or 36 months.

294. The method of embodiment 288 or 289, wherein the uPCR are decreased by at least 5%, 10%, 15%, 20%, 25%, 30%, 35%, 40%, 45%, 50%, 55%, 60%, 65%, 70%, 75%, 80%, 85%, or 90%, e.g., within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after the administration (e.g., after the first, second, or third administration).

295. The method of any of the preceding embodiments, wherein the subject has a disorder, or is at risk of having a disorder.

296. The method of 295, wherein the disorder is associated with decreased kidney function.

297. The method of 295 or 296, wherein the disorder is treatable by improving kidney function.

298. The method of any of embodiments 295-297, wherein the subject is at risk of kidney failure.

299. The method of any of embodiments 295-298, wherein the disorder is a kidney disorder, e.g., a chronic kidney disease (CKD).

300. The method of any of embodiments 295-299, wherein the disorder is an autoimmune disorder, e.g., an autoantibody-related disorder (e.g., an IgM autoantibody-related disorder).

301. The method of embodiment 300, wherein the autoantibody-related disorder is primary membranous nephropathy, Goodpasture's disease, or cold agglutinin disease.

302. The method of any of embodiments 295-301, wherein the disorder is an IgM-mediated disorder (e.g., an IgM neuropathy).

303. The method of any of embodiments 295-302, wherein the disorder is a glomerulonephritis.

304. The method of any of embodiments 295-303, wherein the disease or disorder is IgA nephropathy (IgAN), lupus nephritis, Henoch-Schonlein purpura (HSP, also referred to as IgA Vasculitis (IgAV), e.g., with or without nephritis), vasculitis (e.g., ANCA-associated vasculitis or renal vasculitis), lupus, including systemic lupus erythematosus (SLE) and lupus nephritis, atypical hemolytic uremic syndrome (aHUS), membranoproliferative glomerulonephritis (MPGN), primary membranous nephropathy, Goodpasture's disease, cold agglutinin disease, anti-MAG neuropathy, anti-GM1 neuropathy (multifocal motor neuropathy), Sjogren's syndrome, post transplant IgA nephropathy, post transplant recurrence of glomerular disease, or post transplant IgA vasculitis.

305. The method of embodiment 304, wherein the disease or disorder is IgAN, e.g., primary IgAN or secondary IgAN.

306. The method of embodiment 304, wherein the disease or disorder is Henoch-Schonlein purpura.

307. The method of any of embodiments 304-306, wherein the subject exhibits symptoms of arthritis, symptoms of arthralgia, gastrointestinal symptoms, and/or dermatological symptoms.

308. The method of any of the preceding embodiments, wherein the subject is a human subject, e.g., a human patient.

309. The method of any of the preceding embodiments, wherein the subject is not immunocompromised.

310. The method of embodiment 308 or 309, wherein the subject does not have decreased serum IgG levels relative to an average healthy subject (e.g., wherein the serum IgG levels of the subject are at least 50%, 60%, 70%, 75%, 80%, 85%, 90%, 95%, 96%, 97%, 98%, 99%, or 100% relative to an average healthy subject).

311. The method of any of the preceding embodiments, wherein the subject has proteinuria greater than or equal to a uPCR of 0.75 g/g or urine protein 1 g/day prior to the administration.

312. The method of embodiment 311, wherein the subject has proteinuria lower than 0.1, 0.2, 0.3, 0.4, 0.5, 0.6, or 0.7 g/day after the administration (e.g., within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months of the administration).

313. The method of any of the preceding embodiments, wherein the subject has been treated with a different anti-APRIL antibody molecule.

314. The method of any of the preceding embodiments, further comprising administering a second therapeutic agent or modality to the subject.

315. The method of embodiment 314, wherein the second therapeutic agent comprises a TACI-IgG fusion protein (e.g., telitacicept).

316. The method of embodiment 314, wherein the second therapeutic agent comprises a C1 inhibitor (e.g., a CIs inhibitor, e.g., sutimlimab).

317. The method of embodiment 314, wherein the second therapeutic agent is an anti-CD20 antibody (e.g., rituximab).

The disclosure contemplates all combinations of any one or more of the foregoing aspects and/or embodiments, as well as combinations with any one or more of the embodiments set forth in the detailed description and examples.

Other features, objects, and advantages of the compositions and methods herein will be apparent from the description and drawings, and from the claims.

### BRIEF DESCRIPTION OF THE DRAWINGS

FIG. 1 depicts the mean percent change (±standard deviation) from baseline of aberrantly glycosylated immunoglobulin concentration over time by the indicated treatments (pooled placebo, antibody 2419-1406 at 0.5 mg/kg, antibody 2419-1406 at 2.0 mg/kg, antibody 2419-1406 at 6.0 mg/kg, antibody 2419-1406 at 12.0 mg/kg, placebo+vaccine, and antibody 2419-1406 at 6.0 mg/kg+vaccine) for all patients of any ethnicity (pharmacodynamic population).

FIG. 2 is a series of graphs showing mean percentage change from baseline in aberrantly glycosylated immunoglobulin A (a-g-IgA1; left panel) and immunoglobulin A (IgA; right panel), by treatment.

FIG. 3 is a graph showing tetanus immunoglobulin G (IgG) titer levels in the safety population.

FIG. 4 is a graph showing diphtheria immunoglobulin G (IgG) titer levels in the safety population.

FIG. 5 is a series of graphs showing IgA suppression by mAb 2419-1406 in healthy volunteers. Results are shown for subcutaneous (SC) administration (left panel) and for intravenous (IV) administration (right panel), as indicated.

FIG. 6 depicts participant disposition. PD, pharmacodynamics; PK, pharmacokinetics. ^{a}1 participant lost to follow-up, 1 participant withdrew. ^{b}1 participant lost to follow-up.

FIG. 7 depicts mean serum mAb 2419-1406 concentration over time following a single intravenous dose (pharmacokinetics sample). Values below the lower limit of quantification (LLQ; 0.1 μg/ml) were imputed as the LLQ. SD, standard deviation.

FIG. 8 depicts mean percentage change from baseline and absolute serum concentration for (a) IgA, (b) IgG, (c) IgM, and (d) Gd-IgA₁, by treatment (pharmacodynamics sample). Normal ranges: IgA, 66-433 mg/dl; IgG, 635-1741 mg/dl; IgM, 45-281 mg/dl. Lower limit of quantification for Gd-IgA₁, 0.5 μg/ml. Gd, galactose-deficient; Ig, immunoglobulin.

FIGS. 9A-9B depicts median (IQR) percentage change from baseline in serum (a) APRIL concentration and (b) BAFF concentration, by treatment (pharmacodynamics sample). IQR, interquartile range.

FIGS. 10A-10B depicts (a) tetanus and (b) diphtheria IgG titer levels (vaccinated safety sample). Vaccine administered at the Week 4 visit (Week 4 titers were pre-vaccination). Lower limit of quantification for diphtheria IgG, 0.1 IU/mL. Upper limit of quantification (ULQ) for tetanus IgG, 16.0 IU/mL; for diphtheria IgG, 2.00 IU/mL. Ig, immunoglobulin; IU, international unity; SD, standard deviation.

FIG. 11 depicts (a) tetanus and (b) diphtheria IgM titer levels (vaccinated safety sample). Vaccine administered at the Week 4 visit (Week 4 titers were pre-vaccination). Following this experiment, serum samples from Day 1 were tested post hoc to determine if the differences between groups at Week 4 (pre-vaccination) were related to mAb 2419-1406 treatment. At Day 1, placebo and mAb 2419-1406 6.0 mg/kg groups demonstrated comparable mean anti-tetanus IgM titers (346 EU/mL and 397 EU/mL, respectively) and mean anti-diphtheria IgM titers (670 EU/mL vs 611 EU/mL, respectively), suggesting that the differences at Week 4 were related to mAb 2419-1406 treatment. EU, endotoxin unit; Ig, immunoglobulin; SD, standard deviation.

FIG. 12 depicts (a) tetanus and (b) diphtheria IgA titer levels (vaccinated safety sample). Vaccine administered at the Week 4 visit (Week 4 titers were pre-vaccination). The high mean anti-tetanus IgA titers in the placebo arm were largely driven by one participant. EU, endotoxin unit; Ig, immunoglobulin; SD, standard deviation.

FIGS. 13A-13B are a series of graphs showing the concentrations (μg/mL) of mAb 2419-1406 in the serum of patients over time following a single administration of mAb 2419-1406 at the indicated doses (2 mg/kg, 4 mg/kg, or 8 mg/kg). Results are presented as mean±standard deviation (SD) on a linear (FIG. 13A) or logarithmic (FIG. 13B) scale.

FIGS. 14A-14B are a series of graphs showing the levels of IgA in serum overtime following once monthly administration of placebo or mAb 2419-1406 at the indicated doses (2 mg/kg, 4 mg/kg, or 8 mg/kg). FIG. 14A shows the concentrations (mg/dL) of IgA, and FIG. 14B shows the levels of IgA as a percentage of baseline levels (measured prior to administration of mAb 2419-1406). Baseline levels are set to 100% within each dose group. The dashed line in FIG. 14B represents 40% percent of baseline IgA levels. Results are presented as mean±standard deviation (SD).

FIGS. 15A-15B are a series of graphs showing the levels of IgG in serum overtime following once monthly administration of placebo or mAb 2419-1406 at the indicated doses (2 mg/kg, 4 mg/kg, or 8 mg/kg). FIG. 15A shows the concentrations (mg/dL) of IgG, and FIG. 15B shows the levels of IgG as a percentage of baseline levels (measured prior to administration of mAb 2419-1406). Baseline levels are set to 100% within each dose group. Results are presented as mean±standard deviation (SD).

FIGS. 16A-16B are a series of graphs showing the levels of IgM in serum over time following once monthly administration of placebo or mAb 2419-1406 at the indicated doses (2 mg/kg, 4 mg/kg, or 8 mg/kg). FIG. 16A shows the concentrations (mg/dL) of IgM, and FIG. 16B shows the levels of IgM as a percentage of baseline levels (measured prior to administration of mAb 2419-1406). Baseline levels are set to 100% within each dose group. Results are presented as mean±standard deviation (SD).

FIGS. 17A-17C are a series of graphs showing the change in urine protein:creatinine ratios (uPCR) over time for IgA neuropathy patients administered with placebo or mAb 2419-1406 at the indicated doses (2 mg/kg, 4 mg/kg, or 8 mg/kg). Results are shown as the mean and 95% confidence interval (CI) of the percentage change in uPCR as compared to the baseline time point (t=0, at which the percent change is set to 0). Results for each dose of mAb 2419-1406 are shown separately by dose group (FIG. 17A) or pooled together (FIG. λ5B). FIG. 17C presents the same curves as FIG. 17B overlaid. The dashed lines represent 30% reduction from baseline. The error bar for the month 8 time point in the 2 mg/kg dose group was not shown due to high variability.

FIGS. 18A-18B are a series of graphs showing the change in spot urine protein:creatinine ratios (spot uPCR) from baseline (t=0) by time and dose in patients with baseline spot uPCR≥1.5 g/g (FIG. 18A) or baseline spot uPCR<1.5 g/g (FIG. 18B) prior to administration with placebo or mAb 2419-1406 at the indicated doses (2 mg/kg, 4 mg/kg, and 8 mg/kg). Results are presented as mean and 95% confidence interval (CI) of uPCR change from baseline. The dashed line represents 30% reduction from baseline. In FIG. 18B, the error bars for the month 8 time point in the placebo group, month 7 time point in the 4 mg/kg group, and month 6 and 8 time points in the 8 mg/kg group were not shown due to high variability.

FIG. 19 is a graph showing the estimated glomerular filtration rate (eGFR) by time and dose in patients administered with placebo or mAb 2419-1406 at the indicated doses (2 mg/kg, 4 mg/kg, 8 mg/kg). Results are shown as means+standard deviation (SD) of eGFR, which is expressed as ml/min per body surface area of 1.73 m².

FIG. 20 is a graph showing APRIL levels (μg/mL) in patients administered with placebo or mAb 2419-1406 at the indicated doses (2 mg/kg, 4 mg/kg, or 8 mg/kg) over time. Results are shown as mean+standard deviation (SD).

FIG. 21 is a graph showing levels of galactose-deficient IgA1 (Gd-IgA1) in subjects administered with placebo or mAb 2419-1406 at the indicated doses (2 mg/kg, 4 mg/kg, or 8 mg/kg) over time. Results are shown as mean+standard deviation (SD) of percent baseline.

FIG. 22 is a graph showing percent change from baseline in 24-hour uPCR at Month 9 in pooled mAb 2419-1406 recipients (n=50) versus placebo cohorts (n=17).

FIG. 23 is a graph showing percent change from baseline in 24-hour uPCR at Month 9 in subjects receiving 2 mg/kg (n=17), 4 mg/kg (n=16), or 8 mg/kg (n=17) mAb 2419-1406 or placebo (n=17).

FIG. 24 is a graph showing the change from baseline eGFR (mL/min/1.73 m²) over time in pooled mAb 2419-1406 recipients versus placebo cohorts. Results are shown as mean+standard deviation (SD).

FIG. 25 is a graph showing the mean change from baseline eGFR (mL/min/1.73 m²) over time in subjects administered with placebo or mAb 2419-1406 at the indicated doses (2 mg/kg, 4 mg/kg, or 8 mg/kg).

FIG. 26 is a graph showing the mean change from baseline eGFR (mL/min/1.73 m²) over time in subjects with baseline eGFR≤60 mL/min/1.73 m² and administered with placebo or mAb 2419-1406 at the indicated doses (2 mg/kg, 4 mg/kg, or 8 mg/kg).

FIG. 27 is a graph showing the mean change from baseline eGFR (mL/min/1.73 m²) over time in subjects with baseline proteinuria>2.0 g/day (in which 24-hour urine protein>2.0 g/day or 24-hour uPCR>1.5 g/g) and administered with placebo or mAb 2419-1406 at the indicated doses (2 mg/kg, 4 mg/kg, or 8 mg/kg).

FIG. 28 is a graph showing the mean change from baseline eGFR (mL/min/1.73 m²) over time in subjects with baseline proteinuria>2.0 g/day (in which 24-hour urine protein>2.0 g/day or 24-hour uPCR>1.5 g/g) and baseline eGFR≤60 mL/min/1.73 m², and administered with placebo or mAb 2419-1406 at the indicated doses (2 mg/kg, 4 mg/kg, or 8 mg/kg).

FIG. 29 is a series of graphs showing the levels of total IgA over time following once monthly administration of placebo or mAb 2419-1406 at the indicated doses (2 mg/kg, 4 mg/kg, or 8 mg/kg). Results are expressed as mean+standard deviation (SD) percent baseline IgA. Later time point data were masked to avoid risk of unblinding. The vertical dashed line denoted by “(1)” indicates the last dose (12th dose). The vertical dashed line denoted by “(2)” indicates 30 days after the last dose.

### DETAILED DESCRIPTION

Disclosed herein are antibody molecules that bind to APRIL, e.g., human APRIL, mouse APRIL, or both, with high affinity and specificity. Advantageously, several of the antibody molecules describe herein have improved ability to reduce (e.g., inhibit, block, or neutralize) one or more biological activities of APRIL. Nucleic acid molecules encoding the antibody molecules, expression vectors, host cells, compositions (e.g., pharmaceutical compositions), kits, and methods for making the antibody molecules, are also provided. The antibody molecules and pharmaceutical compositions disclosed herein can be used to improve kidney function, for example, by reversing or preventing progression of decreased kidney function in a subject or by inducing kidney regeneration in a subject. The antibody molecules and pharmaceutical compositions disclosed herein can be used to increase estimated glomerular filtrate rate (eGFR) and/or reduce proteinuria in the kidney of a subject. The antibody molecules and pharmaceutical compositions disclosed herein can be used (alone or in combination with other agents or therapeutic modalities) to treat, prevent and/or diagnose disorders and conditions (e.g., by improving kidney function in a subject having a disorder or condition), e.g., disorders and conditions associated with APRIL, e.g., IgA nephropathy (IgAN) or disorders associated with IgAN (e.g., an advanced chronic kidney disease (CKD), post-transplant IgAN, pediatric IgAN, Henoch-Schonlein purpura (HSP) or cutaneous vasculitis, IgAN with crescentic glomerulonephritis (GN)), IgA vasculitis, IgA dermatitis (e.g., IgA dermatitis herpetiformis, IgA bullous dermatosis), IgM mediated neuropathy (e.g., anti-MAG peripheral neuropathy or IgM mediated neuropathy associated with anti-GM1 antibodies), Waldenstrom's macroglobulinemia (WM, also known as Waldenstrom macroglobulinemia), or lupus nephritis).

IgA nephropathy is one of the most prevalent, chronic glomerular diseases, with a global incidence of approximately 5-50 cases/million (children) and 10-40 cases/million (adults). While typically a relatively indolent disease, IgAN can progress to end-stage renal disease (e.g., kidney failure in 20%-50% of patients within 20 to 30 years). IgA nephropathy patients with minor urine abnormalities, normal blood pressure and normal glomerular filtration rate (GFR) typically need periodic monitoring. For those with more advanced disease, the therapeutic options can include nonspecific treatment to reduce blood pressure and proteinuria by RAS blockade, as well as other general measures, such as lipid lowering, dietary restriction of sodium, smoking cessation and avoidance of NSAIDs and other nephrotoxins.

Without wishing to be bound by theory, it is believed that in some embodiments, the etiology of IgA nephropathy represents a two-hit phenomenon, wherein the first hit occurs in response to a mucosal infection, as production of polymeric IgA1, containing an aberrantly galactosylated hinge region (aberrantly glycosylated IgA1 or a-g IgA1), presents as an autoantigen; and the second hit is the subsequent induction of autoantibodies that results in immune complex formation. These circulating immune complexes are then deposited in the kidney, where complement activation occurs, resulting in promotion of inflammatory pathways, mesangial hyperproliferation, glomerular damage, proteinuria, and progression of kidney disease leading to end-stage renal disease. Without wishing to be bound by theory, it is believed that in some embodiments, reduction of the autoantigen and/or autoantibody, and removal of the resulting immune complexes, and/or mitigation of complement activation can have a beneficial effect on progression of IgA nephropathy and other related diseases and disorders (e.g., an advanced chronic kidney disease (CKD), post-transplant IgAN, pediatric IgAN, Henoch-Schonlein purpura (HSP) or cutaneous vasculitis, IgAN with crescentic glomerulonephritis (GN), IgA vasculitis, IgA dermatitis (e.g., IgA dermatitis herpetiformis, IgA bullous dermatosis), IgM mediated neuropathy (e.g., anti-MAG peripheral neuropathy or IgM mediated neuropathy associated with anti-GM1 antibodies), Waldenstrom's Macroglobulinemia (WM), or lupus nephritis).

Without wishing to be bound by theory, it is believed that in some embodiments, the aberrant biosynthesis of polymeric IgA and antigenic a-g IgA1 is correlated to both disease pathogenesis and progression. In an embodiment, the serum levels of a-g IgA1 are correlated as a heritable trait with substantial heritability in a significant number of adult and pediatric familial IgA nephropathy cases. In an embodiment, a-g IgA1 plays a role in disease pathogenesis, which can be determined, e.g., by ex vivo analysis of peripheral blood mononuclear cells (PBMCs) derived from patients. For example, a-g IgA1 can be secreted in immortalized B cells from IgA nephropathy patients and IgA1 production from patient lymphocytes can be correlated to serum levels of a-g IgA1. As another example, immune complexes derived in part from IgA1-producing cells that were then reconstituted in vitro using sera from IgA nephropathy patients can be pathogenic in mice following passive transfer. Without wishing to be bound by theory, it is believed that in some embodiments, serum levels of a-g IgA1 can be predictive of disease outcomes and provide diagnostic utility as a biomarker for clinical evaluation of disease progression, treatment, and also stratification of patient populations. For example, a targeted reduction in IgA can be therapeutically advantageous and can effectively reduce immune deposits and kidney damage. In an embodiment, treatment with an antibody molecule described herein results in clinically relevant reduction of autoantigen levels, e.g., a-g IgA levels.

Without wishing to be bound by theory, it is believed that in some embodiments, the anti-APRIL antibody molecules described herein (e.g., single doses up to 12.0 mg/kg) are safe and well tolerated in healthy adults. In an embodiment, a single dose of the anti-APRIL antibody molecule can suppress free serum APRIL to the lower level of quantification. In an embodiment, serum a-g IgA1 decreases in parallel with total serum IgA and recovers in a dose-dependent manner following detection of free APRIL in serum.

Without wishing to be bound by theory, it is believed that in some embodiments, the anti-APRIL antibody molecules described herein do not interfere with subjects' ability to mount an antigen-specific serum IgG or IgA boost response to vaccination (e.g., tetanus and diphtheria toxoid vaccination), indicating that qualitative T-cell dependent antibody responses are preserved during APRIL suppression.

### Definitions

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The compositions and methods disclosed herein encompass polypeptides and nucleic acids having the sequences specified, or sequences substantially identical or similar thereto, e.g., sequences at least 85%, 90%, 95% identical or higher to the sequence specified.

In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%, 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a typical embodiment, the length of a reference sequence aligned for comparison purposes is at least 30%, e.g., at least 40%, 50%, 60%, e.g., at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman and Wunsch ((1970) J. Mol. Biol. 48:444-453) algorithm which has been incorporated into the GAP program in the GCG software package (available at gcg.com), using either a Blossum 62 matrix or a PAM250 matrix, and a gap weight of 16, 14, 12, 10, 8, 6, or 4 and a length weight of 1, 2, 3, 4, 5, or 6. In certain embodiments, the percent identity between two nucleotide sequences is determined using the GAP program in the GCG software package (available at gcg.com), using a NWSgapdna.CMP matrix and a gap weight of 40, 50, 60, 70, or 80 and a length weight of 1, 2, 3, 4, 5, or 6. One suitable set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J Mol. Biol. 215:403-10. BLAST nucleotide searches can be performed with the NBLAST program, score=100, wordlength=12 to obtain nucleotide sequences homologous to a nucleic acid as described herein. BLAST protein searches can be performed with the XBLAST program, score=50, wordlength=3 to obtain amino acid sequences homologous to protein molecules described herein.

To obtain gapped alignments for comparison purposes, Gapped BLAST can be utilized as described in Altschul et al., (1997) NucleicAcids Res. 25:3389-3402. When utilizing BLAST and gapped BLAST programs, the default parameters of the respective programs (e.g., XBLAST and NBLAST) can be used.

See ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions 4) are suitable conditions and the ones that should be used unless otherwise specified.

It is understood that the molecules described herein may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide,” “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, “improving kidney function” refers to increase in a metric for kidney functionality relative to a reference subject (e.g., an untreated patient for a disease or disorder as described herein, or the same subject at an earlier time point). An increase in a metric for kidney function may, in some instances, include reduced decrease in the metric for kidney function relative to the reference subject. In some instances, an increase in a metric for kidney function includes stabilization (e.g., cessation of change over time in the metric for kidney function) relative to an earlier measurement of the metric for kidney function in the same subject. In some instances, an increase in a metric for kidney function includes an increase in the metric for kidney function relative to an earlier measurement of the metric for kidney function in the same subject. In some instances, an increase in a metric for kidney function includes reversal of a decline in the metric for kidney function in the same subject. In some instances, a metric for kidney functionality includes estimated glomerular filtration rate (eGFR). In some instances, a metric for kidney functionality includes level of proteinuria in the subject. In some instances, a metric for kidney functionality includes urine protein:creatinine ratio (uPCR), e.g., spot uPCR. In some instances, a metric for kidney functionality includes serum IgA levels. In some instances, a metric for kidney functionality includes serum IgG levels. In some instances, a metric for kidney functionality includes serum IgM levels.

As used herein, the term “treat,” e.g., IgA nephropathy, means that a subject (e.g., a human) who has a disorder, e.g., IgA nephropathy, and/or experiences a symptom of a disorder, e.g., IgA nephropathy, will, in an embodiment, suffer less a severe symptom and/or recover faster when an antibody molecule is administered than if the antibody molecule were never administered. In an embodiment, when IgA nephropathy is treated, a kidney biopsy will show less or no IgA deposits, e.g., in the form of immune complexes in the mesangium of the kidney, after effective treatment for IgA nephropathy. For example, a diagnostic assay using immunofluorescence or electron microscopy will detect less no IgA deposits in a biological sample of a subject after administration of an antibody molecule described herein for the effective treatment of IgA nephropathy. Other assays, urine tests, blood tests, iothalamate clearance tests, or kidney imaging (e.g., ultrasound, X-rays, or cystoscopy), can also be used to monitor treatment in a patient, or to detect the presence, e.g., decreased presence (or absence), of a symptom of IgA nephropathy, after treatment of IgA nephropathy in the subject. Treatment can, e.g., partially or completely, alleviate, ameliorate, relieve, inhibit, or reduce the severity of, and/or reduce incidence, and optionally, delay onset of, one or more manifestations of the effects or symptoms, features, and/or causes of a disorder, e.g., IgA nephropathy. In an embodiment, treatment is of a subject who does not exhibit certain signs of a disorder, e.g., IgA nephropathy, and/or of a subject who exhibits only early signs of a disorder, e.g., nephropathy. In an embodiment, treatment is of a subject who exhibits one or more established signs of a disorder, e.g., IgA nephropathy. In an embodiment, treatment is of a subject diagnosed as suffering from a disorder, e.g., IgA nephropathy.

As used herein, the term “prevent,” a disorder, e.g., IgA nephropathy, means that a subject (e.g., a human) is less likely to have the disorder, e.g., IgA nephropathy, if the subject receives the antibody molecule.

Various aspects of the compositions and methods herein are described in further detail below. Additional definitions are set out throughout the specification.

### April

APRIL (A PRoliferation Inducing Ligand), also known as CD256, TNF- and APOL-related Leukocyte Expressed Ligand 2 (TALL-2), or TNF-related Death Ligand 1 (TRDL-1), is a TNF family cytokine encoded by the Tumor Necrosis Factor Ligand Superfamily Member 13 (TNFSF13) gene (also known as APRIL, TALL2, or ZTNF2). APRIL plays a role in a number of biological processes such as signal transduction, regulation of cell proliferation, and IgA class switching (Hahne et al. (1998) J. Exp. Med. 188:1185-1190 (1998); Castigli et al. Proc. Natd. Acad. Sci. U.S.A. 101:3903-3908 (2004)).

APRIL is both functionally and structurally related to BAFF (B Cell Activating Factor F13B) also known as BLyS (B lymphocyte stimulator). Both cytokines are involved in regulating keys aspects of innate and adaptive immune functions. Both APRIL and BAFF bind the lymphocyte receptors TACI (transmembrane activator and CAML interactor) and BCMA (B cell maturation antigen). APRIL and BAFF appear to heterologously interact with each other through protein-protein interactions. While both APRIL and BAFF share biochemical (receptor binding), immunological and even some structural overlap (e.g., as it relates to the three-dimensional topology of their respective receptor binding domains), the two cytokines, nevertheless, are both structurally and functionally distinct. APRIL binds to biologically relevant heparan sulfate (present in the extracellular matrices of cells as heparan sulfate proteoglycans); BAFF does not. This interaction plays a critical biological function with respect to promoting the oligomerization state of APRIL in concert with its localized interaction with TACI, which likewise requires HSPGS for full activity. Unlike BAFF which acts as a potent activator of B cells inclusive of both proliferation and differentiation, APRIL would appear to function more particularly with respect to the modulation of B cell phenotype, e.g., as it relates to IgA production and the differentiation/survival of IgA positive plasma cells. As such, a targeted disruption in APRIL-receptor signaling is expected to have less perturbative effects on B cell homeostasis and overall immune function in comparison to other immune related therapeutics that target BAFF (e.g., belimumab) or anti CD20 therapies (e.g., rituximab) that largely target pre and early B cells. APRIL has also been shown to be expressed at high levels on other myeloid related cells and lymphoid tissues, as well as hematological cancers (e.g., myeloma, chronic lymphocytic leukemia (CLL)) and solid tumors (e.g., colon, thyroid, and breast).

Exemplary amino acid and nucleotide sequences of human APRIL are described, e.g., in Hahne et al. J. Exp. Med. 188:1185-1190 (1998); Shu et al. J. Leukoc. Biol. 65:680-683 (1999); Kelly et al. Cancer Res. 60:1021-1027(2000); and Pradet-Balade et al. EMBO J 21:5711-5720 (2002).

The amino acid sequence of human APRIL (isoform alpha, also referred to as the “canonical” sequence (SEQ ID NO: 85)) is provided as follows.

>huAPRIL
MPASSPFLLAPKGPPGNMGGPVREPALSVALWLSWGAALGAVACAMALLT
QQTELQSLRREVSRLQGTGGPSQNGEGYPWQSLPEQSSDALEAWENGERS
RKRRAVLTQKOKKQHSVLHLVPINATSKDDSDVTEVMWQPALRRGRGLQA
QGYGVRIQDAGVYLLYSQVLFQDVTFTMGQVVSREGQGRQETLFRCIRSM
PSHPDRAYNSCYSAGVFHLHQGDILSVIIPRARAKLNLSPHGTFLGFVKL

There are several isoforms of human APRIL produced by alternative splicing.

Isoform beta has the following amino acid sequence (SEQ ID NO: 86):

>sp|O75888-2|TNF13_HUMAN Isoform Beta of Tumor
necrosis factor ligand superfamily member 13
OS = Homo sapiens GN = TNFSF13
MPASSPFLLAPKGPPGNMGGPVREPALSVALWLSWGAALGAVACAMALLT
QQTELQSLRREVSRLQGTGGPSQNGEGYPWQSLPEQSSDALEAWENGERS
RKRRAVLTQKQKNDSDVTEVMWQPALRRGRGLQAQGYGVRIQDAGVYLLY
SQVLFQDVTFTMGQVVSREGQGRQETLFRCIRSMPSHPDRAYNSCYSAGV
FHLHQGDILSVIIPRARAKLNLSPHGTFLGFVKL

The sequence of isoform beta differs from the canonical sequence as follows: amino acids 113-129 of SEQ ID NO: 85: KQHSVLHLVPINATSKD (SEQ ID NO: 347)—N Isoform gamma has the following amino acid sequence (SEQ ID NO: 87):

>sp|O75888-3|TNF13_HUMAN Isoform Gamma of Tumor
necrosis factor ligand superfamily member 13
OS = Homo sapiens GN = TNFSF13
MPASSPFLLAPKGPPGNMGGPVREPALSVALWLSWGAALGAVACAMALLT
QQTELQSLRREVSRLQGTGGPSQNGEGYPWQSLPEQSSDALEAWENGERS
RKRRAVLTQKQKKQHSVLHLVPINATSKDDSDVTEVMWQPALRRGRGLQA
QGYGVRIQDAGVYLLYSQVLFQDVTFTMGQVVSREGQGRQETLFRCIRSM
PSHPDRAYNSCYSAGVFHLHQGDILSVIIPRARAKLNLSPHGTFLGL

The sequence of isoform gamma differs from the canonical sequence as follows: amino acids 247-249: Missing.

Isoform 4 has the following amino acid sequence (SEQ ID NO: 88):

>sp|O75888-4|TNF13_HUMAN Isoform 4 of Tumor
necrosis factor ligand superfamily member 13
OS = Homo sapiens GN = TNFSF13
MPASSPFLLAPKGPPGNMGGPVREPALSVALWLSWGAALGAVACAMALLT
QQTELQSLRREVSRLQGTGGPSQNGEGYPWQSLPEQHSVLHLVPINATSK
DDSDVTEVMWQPALRRGRGLQAQGYGVRIQDAGVYLLYSQVLFQDVTFTM
GQVVSREGQGRQETLFRCIRSMPSHPDRAYNSCYSAGVFHLHQGDILSVI
IPRARAKLNLSPHGTFLGFVKL

The sequence of isoform 4 differs from the canonical sequence as follows: amino acids 86-113: Missing.

Isoform TWE-PRIL has the following amino acid sequence (SEQ ID NO: 89):

>sp|O43508-2|TNF12_HUMAN Isoform TWE-PRIL of
Tumor necrosis factor ligand superfamily
member 12 OS = Homo sapiens GN = TNFSF12
MAARRSQRRRGRRGEPGTALLVPLALGLGLALACLGLLLAVVSLGSRASL
SAQEPAQEELVAEEDQDPSELNPQTEESQDPAPFLNRLVRPRRSAPKGRK
TRARRAIAAHYEVHPRPGQDGAQAGVDGTVSGWEEARINSSSPLRYNRQI
GEFIVTRAGLYYLYCOSSDALEAWENGERSRKRRAVLTQKQKKQHSVLHL
VPINATSKDDSDVTEVMWQPALRRGRGLQAQGYGVRIQDAGVYLLYSQVL
FQDVTFTMGQVVSREGQGRQETLFRCIRSMPSHPDRAYNSCYSAGVFHLH
QGDILSVIIPRARAKLNLSPHGTFLGFVKL

Isoform 5 has the following amino acid sequence (SEQ ID NO: 90):

>sp|O75888-5|TNF13_HUMAN Isoform 5 of Tumor
necrosis factor ligand superfamily member
13 OS = Homo sapiens GN = TNFSF13
MGGPVREPALSVALWLSWGAALGAVACAMALLTQQTELQSLRREVSRLQG
TGGPSQNGEGYPWQSLPEQHSVLHLVPINATSKDDSDVTEVMWQPALRRG
RGLQAQGYGVRIQDAGVYLLYSQVLFQDVTFTMGQVVSREGQGRQETLFR
CIRSMPSHPDRAYNSCYSAGVFHLHQGDILSVIIPRARAKLNLSPHGTFL
GFVKL

The sequence of isoform 5 differs from the canonical sequence as follows: amino acids 1-17: Missing; amino acids 87-114: Missing.

Other variant and alternative sequences of human APRIL are described, e.g., in The MGC Project Team, Genome Res. 14:2121-2127 (2004); Ota et al. Nat. Genet. 36:40-45 (2004); and Kelly et al. Cancer Res. 60:1021-1027 (2000).

As used herein, when an anti-APRIL antibody molecule binds, or substantially binds, to human APRIL, it binds, or substantially binds, to one or more isoforms of human APRIL, e.g., one or more isoforms of human APRIL described herein. In an embodiment, the antibody molecule binds or substantially binds to human APRIL having the amino acid sequence of SEQ ID NO: 85.

Exemplary amino acid and nucleotide sequences of mouse APRIL are described, e.g., in Yu et al. Nat. Immunol. 1:252-256 (2000); Carninci et al. Science 309:1559-1563 (2005); The MGC Project Team, Genome Res. 14:2121-2127 (2004); and Bossen et al. J. Biol Chem. 281: 13964-13971 (2006).

The amino acid sequence of mouse APRIL isoform 1 (SEQ ID NO: 91) is provided as follows.

>muAPRIL
MPASSPGHMGGSVREPALSVALWLSWGAVLGAVTCAVALLIQQTELQSLR
REVSRLQRSGGPSQKQGERPWQSLWEQSPDVLEAWKDGAKSRRRRAVLTQ
KHKKKHSVLHLVPVNITSKADSDVTEVMWQPVLRRGRGLEAQGDIVRVWD
TGIYLLYSQVLFHDVTFTMGQVVSREGQGRRETLFRCIRSMPSDPDRAYN
SCYSAGVFHLHQGDIITVKIPRANAKLSLSPHGTFLGFVKL

The amino acid sequence of mouse APRIL isoform 2 (SEQ ID NO: 92) is provided as follows.

MPASSPGHMGGSVREPALSVALWLSWGAVLGAVTCAVALLIQQTELQSLR
REVSRLQRSGGPSQKQGERPWQSLWEQSPDVLEAWKDGAKSRRRRAVLTQ
KHKKKHSVLHLVPVNITSKDSDVTEVMWQPVLRRGRGLEAQGDIVRVWDT
GIYLLYSQVLFHDVTFTMGQVVSREGQGRRETLFRCIRSMPSDPDRAYNS
CYSAGVFHLHQGDIITVKIPRANAKLSLSPHGTFLGFVKL

As used herein, when an anti-APRIL antibody molecule binds, or substantially binds, to mouse APRIL, it binds, or substantially binds, to one or more isoforms of mouse APRIL, e.g., one or more isoforms of mouse APRIL described herein. In an embodiment, the antibody molecule binds or substantially binds to mouse APRIL having the amino acid sequence of SEQ ID NO: 91, SEQ ID NO: 92, or both.

As used herein, when an anti-APRIL antibody molecule does not bind, or does not substantially bind, to mouse APRIL, it does not bind, or does not substantially bind, to one or more isoforms of mouse APRIL, e.g., one or more isoforms of mouse APRIL described herein. In an embodiment, the antibody molecule does not bind, or does not substantially bind, to mouse APRIL having the amino acid sequence of SEQ ID NO: 91 or 92. In a typical embodiment, the antibody molecule does not bind, or does not substantially bind, to mouse APRIL having the amino acid sequence of SEQ ID NO: 91 and mouse APRIL having the amino acid sequence of SEQ ID NO: 92.

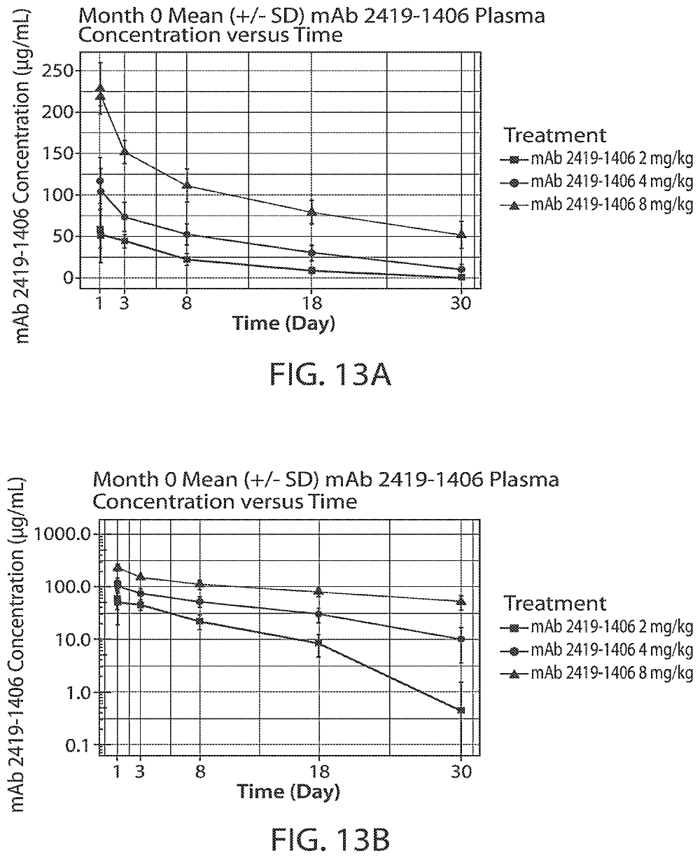

Sequence alignment of exemplary human and mouse APRIL proteins (SEQ ID NOS: 85 and 91, respectively) is shown in FIG. 13 of International Application Publication No. WO2017/091683, the contents of which are incorporated herein by reference in its entirety.

### Antibody Molecules

Disclosed herein are antibody molecules that bind to APRIL, e.g., an APRIL molecule described herein.

As used herein, the term “antibody molecule” refers to a protein, e.g., an immunoglobulin chain or a fragment thereof, comprising at least one immunoglobulin variable domain sequence. The term “antibody molecule” includes, for example, full-length, mature antibodies and antigen-binding fragments of an antibody. For example, an antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The antibody molecules can be monoclonal or polyclonal. The antibody molecule can also be a human, humanized, CDR-grafted, or in vitro generated antibody. The antibody molecule can have a heavy chain constant region chosen from, e.g., IgG1, IgG2, IgG3, or IgG4. The antibody molecule can also have a light chain chosen from, e.g., kappa or lambda. The term “immunoglobulin” (Ig) is used interchangeably with the term “antibody” herein.

Examples of antigen-binding fragments include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments may be obtained using any suitable method, including several conventional techniques known to those with skill in the art, and the fragments can be screened for utility in the same manner as are intact antibodies.

The term “antibody” includes intact molecules as well as functional fragments thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

The antibody molecule can be a single chain antibody. A single-chain antibody (scFv) may be engineered (see, for example, Colcher, D. et al. (1999) Ann NY Acad Sci 880:263-80; and Reiter, Y. (1996) Clin Cancer Res 2:245-52). The single chain antibody can be dimerized or multimerized to generate multivalent antibodies having specificities for different epitopes of the same target protein.

The antibody molecules disclosed herein can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to some aspects, a single domain antibody is a naturally occurring single domain antibody known as heavy chain antibody devoid of light chains. Such single domain antibodies are disclosed in WO 94/04678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are also contemplated.

The VH and VL regions can be subdivided into regions of hypervariability, termed “complementarity determining regions” (CDR), interspersed with regions that are more conserved, termed “framework regions” (FR or FW). The terms “complementarity determining region,” and “CDR,” as used herein refer to the sequences of amino acids within antibody variable regions which confer antigen specificity and binding affinity. As used herein, the terms “framework,” “FW” and “FR” are used interchangeably.

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains.

In: Antibody Engineering Lab Manual (Ed.: Duebel, S. and Kontermann, R., Springer-Verlag, Heidelberg). In an embodiment, the following definitions are used: AbM definition of CDR1 of the heavy chain variable domain and Kabat definitions for the other CDRs. In an embodiment, Kabat definitions are used for all CDRs. In addition, embodiments described with respect to Kabat or AbM CDRs may also be implemented using Chothia hypervariable loops. Each VH and VL typically includes three CDRs and four FRs, arranged from amino-terminus to carboxy-terminus in the following order: FRI, CDR1, FR2, CDR2, FR3, CDR3, and FR4.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding region” refers to the part of an antibody molecule that comprises determinants that form an interface that binds to an antigen, e.g., APRIL, or an epitope thereof. With respect to proteins (or protein mimetics), the antigen-binding region typically includes one or more loops (of at least, e.g., four amino acids or amino acid mimics) that form an interface that binds to the antigen, e.g., APRIL. Typically, the antigen-binding region of an antibody molecule includes at least one or two CDRs and/or hypervariable loops, or more typically at least three, four, five or six CDRs and/or hypervariable loops.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

The antibody molecule can be a polyclonal or a monoclonal antibody. In some embodiments, the antibody can be recombinantly produced, e.g., produced by any suitable phage display or combinatorial methods.

Various phage display and combinatorial methods for generating antibodies are known in the art (as described in, e.g., Ladner et al. U.S. Pat. No. 5,223,409; Kang et al. International Publication No.

WO 92/18619; Dower et al. International Publication No. WO 91/17271; Winter et al. International Publication WO 92/20791; Markland et al. International Publication No. WO 92/15679; Breitling et al.

International Publication WO 93/01288; McCafferty et al. International Publication No. WO 92/01047; Garrard et al. International Publication No. WO 92/09690; Ladner et al. International Publication No.

WO 90/02809; Fuchs et al. (1991) Bio Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio Technology 9:1373-1377; Hoogenboom et al. (1991) Nuc Acid Res 19:4133-4137; and Barbas et al. (1991) PNAS 88:7978-7982, the contents of all of which are incorporated by reference herein).

In an embodiment, the antibody molecule is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. In an embodiment, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by any suitable recombinant DNA technique. Several are known in the art (see Robinson et al., International Patent Application Publication No. WO1987/002671; Akira, et al., European Patent Application Publication No. 184,187; Taniguchi, M., European Patent Application Publication No. 171,496; Morrison et al., European Patent Application Publication No. 173,494; Neuberger et al., International Patent Application Publication No. WO86/01533; Cabilly et al.

U.S. Pat. No. 4,816,567; Cabilly et al., European Patent Application Publication No. 125,023; Better et al. (1988 Science 240:1041-1043); Liu et al. (1987) PNAS 84:3439-3443; Liu et al., 1987, J Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J Natl Cancer Inst. 80:1553-1559).

A humanized or CDR-grafted antibody will have at least one or two but generally all three recipient CDRs (of heavy and or light immunoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to lipopolysaccharide. In an embodiment, the donor will be a rodent antibody, e.g., a rat or mouse antibody, and the recipient will be a human framework or a human consensus framework.

Typically, the immunoglobulin providing the CDRs is called the “donor” and the immunoglobulin providing the framework is called the “acceptor.” In some embodiments, the donor immunoglobulin is a non-human (e.g., rodent). The acceptor framework is typically a naturally-occurring (e.g., a human) framework or a consensus framework, or a sequence about 85% or higher, e.g., 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by any suitable method, and several such methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare humanized antibodies (UK Patent Application GB 2188638A, filed on Mar. 26, 1987; Winter U.S. Pat. No. 5,225,539), the contents of which is expressly incorporated by reference.

Also provided are humanized antibodies in which specific amino acids have been substituted, deleted or added. Criteria for selecting amino acids from the donor are described in, e.g., U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In an embodiment, the antibody molecule has a heavy chain constant region chosen from, e.g., the heavy chain constant regions of IgG1, IgG2 (e.g., IgG2a), IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody molecule (e.g., to increase or decrease one or more of: Fc receptor binding, antibody glycosylation, the number of cysteine residues, effector cell function, and/or complement function). In an embodiment, the antibody molecule has effector function and can fix complement. In another embodiment, the antibody molecule does not recruit effector cells or fix complement. In certain embodiments, the antibody molecule has reduced or no ability to bind an Fc receptor. For example, it may be an isotype or subtype, fragment or other mutant, which does not support binding to an Fc receptor, e.g., it has a mutagenized or deleted Fc receptor binding region.

In an embodiment, a constant region of the antibody molecule is altered. Methods for altering an antibody constant region are known in the art. Antibody molecules s with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Amino acid mutations which stabilize antibody structure, such as S228P (EU nomenclature, S241P in Kabat nomenclature) in human IgG4 are also contemplated. Similar type of alterations could be described which if applied to the murine, or other species immunoglobulin would reduce or eliminate these functions.

In an embodiment, the antibody molecule comprises an Fc region that comprise one or more (e.g., 2, 3,4, 5, 6,7, 8, 9, 10, 11, 12, 13, 14, 15, 16, 17, 18, 19, 20, 21, 22, 23, 24, 25, or more) of mutations or combinations of mutations described in Table 6.

TABLE 6
Exemplary Fc mutations

| Name | Mutation (according to EU numbering) |
|---|---|
| FcMut001 | I253M |
| FcMut002 | L309H_D312A_N315D |
| FcMut003 | L309N |
| FcMut004 | M252E_S254R |
| FcMut005 | M252E_S254R_R255Y |
| FcMut006 | S254H |
| FcMut007 | S254M |
| FcMut008 | T256D_T307R |
| FcMut009 | T256L_N286I_T307I |
| FcMut010 | T256I_N286I_T307I |
| FcMut011 | K248S_D376Q |
| FcMut012 | K248S_D376N |
| FcMut013 | D376Q_E380A |
| FcMut014 | D376N_E380A |
| FcMut015 | D376Q_M428L |
| FcMut016 | K248S_A378I |
| FcMut017 | L314K |
| FcMut018 | T250Q_M428L |
| FcMut019 | M428L_N434A |
| FcMut020 | N434A |
| FcMut021 | T307A_E380A_N434A |
| FcMut022 | M252W |
| FcMut023 | V308F |
| FcMut024 | V308F_N434Y |
| FcMut026 | T256D_T307R_D376N |
| FcMut027 | L309R_D312E |
| FcMut028 | L309R_Q311P_D312E |
| FcMut029 | K246N_P247A |
| FcMut030 | K246N_P247A_D376N |
| FcMut031 | T256E_T307R |
| FcMut032 | T256R_T307D |
| FcMut033 | T256R_T307E |
| FcMut034 | Q311P |
| FcMut035 | D376Q |
| FcMut036 | L234A_L235A |
| FcMut037 | L235V_G236A |
| FcMut038 | L234P_L235P |
| FcMut039 | L235P |
| FcMut040 | P329G |
| FcMut041 | P329E |
| FcMut042 | E233K |
| FcMut043 | T256D_N286D_A287S_T307R |
| FcMut044 | T256D_P257L_T307R |
| FcMut045 | T256D_T307R_Q311V |
| FcMut046 | P247D_T256D_T307R |
| FcMut047 | P247D_N286D_A287S_Q311V |
| FcMut048 | P257M_V308N |
| FcMut049 | V279I_Q311L_N315T |
| FcMut050 | M428L_N434S |
| FcMut051 | N434S |
| FcMut052 | H433G_N434P |
| FcMut053 | V259I_V308F_M428L |
| FcMut067 | T256D_N286D_T307R |
| FcMut068 | T256D_N286E_T307R |
| FcMut069 | T256D_N286Q_T307R |
| FcMut070 | T256D_P257T_T307R |
| FcMut071 | T256D_P257V_T307R |
| FcMut072 | T256D_T307R_Q311I |
| FcMut073 | T256D_T307R_Q311L |
| FcMut074 | T256D_T307R_Q311M |
| FcMut075 | T256D_P257L_N286D_T307R_Q311V |
| FcMut076 | T256D_T307R_M428L |
| FcMut077 | M428L |
| FcMut078 | M252Y_S254T_T256Q |
| FcMut079 | M252Y_S254T_T256E_K288E |
| FcMut080 | T256K_K288E |
| FcMut081 | T256D_E258T |
| FcMut082 | E283Q_H285E |
| FcMut083 | R344D_D401R |
| FcMut084 | K248E_E380K |
| FcMut085 | K248E_E380R |
| FcMut086 | K246H |
| FcMut087 | K248H |
| FcMut088 | T250I |
| FcMut089 | T250V |
| FcMut090 | L251F |
| FcMut091 | L251M |
| FcMut093 | P257V |
| FcMut094 | N276D |
| FcMut095 | H285N |
| FcMut096 | H285D |
| FcMut097 | K288H |
| FcMut098 | K288Q |
| FcMut099 | K288E |
| FcMut100 | T307E |
| FcMut101 | T307Q |
| FcMut102 | V308P |
| FcMut103 | V308I |
| FcMut104 | V308L |
| FcMut105 | L309H |
| FcMut106 | L309M |
| FcMut107 | Q311H |
| FcMut108 | L314F |
| FcMut109 | Y319H |
| FcMut110 | I336T |
| FcMut111 | P343D |
| FcMut112 | P343V |
| FcMut113 | E345Q |
| FcMut114 | P346V |
| FcMut115 | P374T |
| FcMut116 | D376N |
| FcMut117 | A378S |
| FcMut118 | A431T |
| FcMut119 | A431P |
| FcMut120 | A431G |
| FcMut121 | L432V |
| FcMut122 | L432I |
| FcMut123 | L432Q |
| FcMut124 | N434T |
| FcMut125 | H435N |
| FcMut126 | Y436H |
| FcMut127 | K439Q |
| FcMut128 | T256D |
| FcMut129 | T307R |
| FcMut130 | A378T |
| FcMut131 | A378D |
| FcMut132 | A378H |
| FcMut133 | A378Y |
| FcMut134 | A378V |
| FcMut135 | D376R |
| FcMut136 | D376F |
| FcMut137 | D376W |
| FcMut138 | L314H |
| FcMut139 | L432E_T437Q |
| FcMut140 | D376Q_A378T |
| FcMut141 | D376Q_I377M_A378T |
| FcMut142 | P244Q_D376Q |
| FcMut143 | P247T_A378T |
| FcMut144 | P247N_A378T |
| FcMut145 | T256D_T307R_L309T |
| FcMut146 | A339T_S375E_F404Y |
| FcMut147 | L235V_G236A_T256D_T307R |
| FcMut148 | L235V_G236A_D376Q_M428L |
| FcMut149 | L314N |
| FcMut150 | N315D |
| FcMut151 | A378T |
| FcMut152 | T437Q |
| FcMut153 | L432E |
| FcMut154 | Y436R |
| FcMut155 | L314M |
| FcMut156 | L234A_L235A_T256D_T307R_Q311V |
| FcMut157 | L234A_L235A_T256D_P257V_T307R |
| FcMut158 | L234A_L235A_T256D_P257L_N286D_T307R_Q311V |
| FcMut159 | L235V_G236A_T256D_T307R_Q311V |
| FcMut160 | L235V_G236A_T256D_P257V_T307R |
| FcMut161 | L235V_G236A_T256D_P257L_N286D_T307R_Q311V |
| FcMut162 | S267T_A327N_A330M |
| FcMut163 | S267T_A327N |
| FcMut164 | L235V_G236A_S267T_A327N_A330M |
| FcMut165 | L235V_G236A_S267T_A327N |
| FcMut166 | M252Y_S254T |
| FcMut167 | T256E |
| FcMut168 | G236A_1332E |
| FcMut169 | S239D_I332E |
| FcMut170 | G236A_S239D_I332E |
| FcMut171 | T256D_N286D_T307R_Q311V |
| FcMut172 | T256D_E258T_T307R |
| FcMut173 | T256D_E258T_T307R_Q311V |
| FcMut174 | T256D_P257V_E258T_T307R |
| FcMut175 | T256D_P257L_E258T_N286D_T307R_Q311V |
| FcMut176 | T256D_E258T_N286D_T307R_Q311V |
| FcMut177 | A378V_M428L |
| FcMut178 | A378V_M428I |
| FcMut179 | A378V_M428V |
| FcMut180 | T256D_N286D |
| FcMut181 | T256D_A378V |
| FcMut182 | T256D_Q311V |
| FcMut183 | T256D_Q311V_A378V |
| FcMut184 | T256D_T307R_A378V |
| FcMut185 | T256D_N286D_T307R_A378V |
| FcMut186 | T256D_T307R_Q311V_A378V |
| FcMut187 | H285D_A378V |
| FcMut188 | H285D_Q311V |
| FcMut189 | T256D_H285D |
| FcMut190 | T256D_H285D_Q311V |
| FcMut191 | T256D_H285D_T307R |
| FcMut192 | T256D_H285D_T307R_A378V |
| FcMut193 | H285D_L314M_A378V |
| FcMut194 | T256D_E258T_H285D_Q311H |
| FcMut195 | T256D_E258T_H285D |
| FcMut196 | H285D_N315D |
| FcMut197 | H285N_T307Q_N315D |
| FcMut198 | H285D_L432E_T437Q |
| FcMut199 | T256D_E258T_N315D |
| FcMut200 | P257V_H285N |
| FcMut201 | H285N_L432F |
| FcMut202 | H285N_T437I |
| FcMut203 | T256D_E258T_L314M |
| FcMut204 | T256D_E258T_T307Q |
| FcMut205 | T256D_E258T_A378V |
| FcMut206 | V308P_A378V |
| FcMut207 | P257V_A378T |
| FcMut208 | P257V_V308P_A378V |
| FcMut209 | N315D_A378T |
| FcMut210 | H285N_L314M |
| FcMut211 | L314M_L432E_T437Q |
| FcMut212 | T307Q_N315D |
| FcMut213 | H285D_T307Q_A378V |
| FcMut214 | L314M_N315D |
| FcMut215 | T307Q_Q311V_A378V |
| FcMut216 | H285D_Q311V_A378V |
| FcMut217 | Q311V_N315D_A378V |
| FcMut218 | T256D_E258T_Q311V |
| FcMut219 | T256D_N315D_A378V |
| FcMut220 | T256D_Q311V_N315D |
| FcMut221 | T256D_T307Q_A378V |
| FcMut222 | T256D_T307Q_Q311V |
| FcMut223 | T256D_H285D_A378V |
| FcMut224 | T256D_H285D_T307R_Q311V |
| FcMut225 | T256D_H285D_N286D_T307R |
| FcMut226 | T256D_H285D_N286D_T307R_Q311V |
| FcMut227 | T256D_H285D_N286D_T307R_A378V |
| FcMut228 | T256D_N286D_T307R_Q311V_A378V |
| FcMut229 | T256D_H285D_T307R_Q311V_A378V |
| FcMut230 | V308P_Q311V_A378V |
| FcMut231 | T256D_V308P_A378V |
| FcMut232 | T256D_V308P_Q311V |
| FcMut233 | T256D_E258T_V308P |
| FcMut234 | H285D_V308P_Q311V |
| FcMut242 | E258T |
| FcMut243 | N286D |
| FcMut244 | Q311V |
| YTE | M252Y_S254T_T256E |

In an embodiment, the Fe region comprises FcMut001. In an embodiment, the Fe region comprises FcMut002. In an embodiment, the Fe region comprises FcMut003. In an embodiment, the Fe region comprises FcMut004. In an embodiment, the Fe region comprises FcMut005. In an embodiment, the Fe region comprises FcMut006. In an embodiment, the Fe region comprises FcMut007. In an embodiment, the Fe region comprises FcMut008. In an embodiment, the Fe region comprises FcMut009. In an embodiment, the Fe region comprises FcMut010. In an embodiment, the Fe region comprises FcMut011. In an embodiment, the Fe region comprises FcMut012. In an embodiment, the Fe region comprises FcMut013. In an embodiment, the Fe region comprises FcMut014. In an embodiment, the Fe region comprises FcMut015. In an embodiment, the Fe region comprises FcMut016. In an embodiment, the Fe region comprises FcMut017. In an embodiment, the Fe region comprises FcMut018. In an embodiment, the Fe region comprises FcMut019. In an embodiment, the Fe region comprises FcMut020. In an embodiment, the Fe region comprises FcMut021. In an embodiment, the Fe region comprises FcMut022. In an embodiment, the Fe region comprises FcMut023. In an embodiment, the Fe region comprises FcMut024. In an embodiment, the Fe region comprises FcMut026. In an embodiment, the Fe region comprises FcMut027. In an embodiment, the Fe region comprises FcMut028. In an embodiment, the Fe region comprises FcMut029. In an embodiment, the Fe region comprises FcMut030. In an embodiment, the Fe region comprises FcMut031. In an embodiment, the Fe region comprises FcMut032. In an embodiment, the Fe region comprises FcMut033. In an embodiment, the Fe region comprises FcMut034. In an embodiment, the Fe region comprises FcMut035. In an embodiment, the Fe region comprises FcMut036. In an embodiment, the Fe region comprises FcMut037. In an embodiment, the Fe region comprises FcMut038. In an embodiment, the Fe region comprises FcMut039. In an embodiment, the Fe region comprises FcMut040. In an embodiment, the Fe region comprises FcMut041. In an embodiment, the Fe region comprises FcMut042. In an embodiment, the Fe region comprises FcMut043. In an embodiment, the Fe region comprises FcMut044. In an embodiment, the Fe region comprises FcMut045. In an embodiment, the Fe region comprises FcMut046. In an embodiment, the Fe region comprises FcMut047. In an embodiment, the Fe region comprises FcMut048. In an embodiment, the Fe region comprises FcMut049. In an embodiment, the Fe region comprises FcMut050. In an embodiment, the Fe region comprises FcMut051. In an embodiment, the Fe region comprises FcMut052. In an embodiment, the Fe region comprises FcMut053. In an embodiment, the Fe region comprises FcMut067. In an embodiment, the Fe region comprises FcMut068. In an embodiment, the Fe region comprises FcMut069. In an embodiment, the Fe region comprises FcMut070. In an embodiment, the Fe region comprises FcMut071. In an embodiment, the Fe region comprises FcMut072. In an embodiment, the Fe region comprises FcMut073. In an embodiment, the Fe region comprises FcMut074. In an embodiment, the Fe region comprises FcMut075. In an embodiment, the Fe region comprises FcMut076. In an embodiment, the Fe region comprises FcMut077. In an embodiment, the Fe region comprises FcMut078. In an embodiment, the Fe region comprises FcMut079. In an embodiment, the Fe region comprises FcMut080. In an embodiment, the Fe region comprises FcMut081. In an embodiment, the Fe region comprises FcMut082. In an embodiment, the Fe region comprises FcMut083. In an embodiment, the Fe region comprises FcMut084. In an embodiment, the Fe region comprises FcMut085. In an embodiment, the Fe region comprises FcMut086. In an embodiment, the Fe region comprises FcMut087. In an embodiment, the Fe region comprises FcMut088. In an embodiment, the Fe region comprises FcMut089. In an embodiment, the Fe region comprises FcMut090. In an embodiment, the Fe region comprises FcMut091. In an embodiment, the Fe region comprises FcMut093. In an embodiment, the Fe region comprises FcMut094. In an embodiment, the Fe region comprises FcMut095. In an embodiment, the Fe region comprises FcMut096. In an embodiment, the Fe region comprises FcMut097. In an embodiment, the Fe region comprises FcMut098. In an embodiment, the Fe region comprises FcMut099. In an embodiment, the Fe region comprises FcMut100. In an embodiment, the Fe region comprises FcMut101. In an embodiment, the Fe region comprises FcMut102. In an embodiment, the Fe region comprises FcMut103. In an embodiment, the Fe region comprises FcMut104. In an embodiment, the Fe region comprises FcMut105. In an embodiment, the Fe region comprises FcMut106. In an embodiment, the Fe region comprises FcMut107. In an embodiment, the Fe region comprises FcMut108. In an embodiment, the Fe region comprises FcMut109. In an embodiment, the Fe region comprises FcMut110. In an embodiment, the Fe region comprises FcMut1 11. In an embodiment, the Fe region comprises FcMut112. In an embodiment, the Fe region comprises FcMut113. In an embodiment, the Fe region comprises FcMut114. In an embodiment, the Fe region comprises FcMut115. In an embodiment, the Fe region comprises FcMut116. In an embodiment, the Fe region comprises FcMut117. In an embodiment, the Fe region comprises FcMut118. In an embodiment, the Fe region comprises FcMut119. In an embodiment, the Fe region comprises FcMut120. In an embodiment, the Fe region comprises FcMut121. In an embodiment, the Fe region comprises FcMut122. In an embodiment, the Fe region comprises FcMut123. In an embodiment, the Fe region comprises FcMut124. In an embodiment, the Fe region comprises FcMut125. In an embodiment, the Fe region comprises FcMut126. In an embodiment, the Fe region comprises FcMut127. In an embodiment, the Fe region comprises FcMut128. In an embodiment, the Fe region comprises FcMut129. In an embodiment, the Fe region comprises FcMut130. In an embodiment, the Fe region comprises FcMut131. In an embodiment, the Fe region comprises FcMut132. In an embodiment, the Fe region comprises FcMut133. In an embodiment, the Fe region comprises FcMut134. In an embodiment, the Fe region comprises FcMut135. In an embodiment, the Fe region comprises FcMut136. In an embodiment, the Fe region comprises FcMut137. In an embodiment, the Fe region comprises FcMut138. In an embodiment, the Fe region comprises FcMut139. In an embodiment, the Fc region comprises FcMut140. In an embodiment, the Fe region comprises FcMut141. In an embodiment, the Fe region comprises FcMut142. In an embodiment, the Fe region comprises FcMut143. In an embodiment, the Fe region comprises FcMut144. In an embodiment, the Fe region comprises FcMut145. In an embodiment, the Fe region comprises FcMut146. In an embodiment, the Fe region comprises FcMut147. In an embodiment, the Fe region comprises FcMut148. In an embodiment, the Fe region comprises FcMut149. In an embodiment, the Fe region comprises FcMut150. In an embodiment, the Fe region comprises FcMut151. In an embodiment, the Fe region comprises FcMut152. In an embodiment, the Fe region comprises FcMut153. In an embodiment, the Fe region comprises FcMut154. In an embodiment, the Fe region comprises FcMut155. In an embodiment, the Fe region comprises FcMut156. In an embodiment, the Fe region comprises FcMut157. In an embodiment, the Fe region comprises FcMut158. In an embodiment, the Fe region comprises FcMut159. In an embodiment, the Fe region comprises FcMut160. In an embodiment, the Fe region comprises FcMut161. In an embodiment, the Fe region comprises FcMut162. In an embodiment, the Fe region comprises FcMut163. In an embodiment, the Fe region comprises FcMut164. In an embodiment, the Fe region comprises FcMut165. In an embodiment, the Fe region comprises FcMut166. In an embodiment, the Fe region comprises FcMut167. In an embodiment, the Fe region comprises FcMut168. In an embodiment, the Fe region comprises FcMut169. In an embodiment, the Fe region comprises FcMut170. In an embodiment, the Fe region comprises FcMut171. In an embodiment, the Fe region comprises FcMut172. In an embodiment, the Fe region comprises FcMut173. In an embodiment, the Fe region comprises FcMut174. In an embodiment, the Fe region comprises FcMut175. In an embodiment, the Fe region comprises FcMut176. In an embodiment, the Fe region comprises FcMut177. In an embodiment, the Fe region comprises FcMut178. In an embodiment, the Fe region comprises FcMut179. In an embodiment, the Fe region comprises FcMut180. In an embodiment, the Fe region comprises FcMut181. In an embodiment, the Fe region comprises FcMut182. In an embodiment, the Fe region comprises FcMut183. In an embodiment, the Fe region comprises FcMut184. In an embodiment, the Fe region comprises FcMut185. In an embodiment, the Fe region comprises FcMut186. In an embodiment, the Fe region comprises FcMut187. In an embodiment, the Fe region comprises FcMut188. In an embodiment, the Fe region comprises FcMut189. In an embodiment, the Fe region comprises FcMut190. In an embodiment, the Fe region comprises FcMut191. In an embodiment, the Fe region comprises FcMut192. In an embodiment, the Fe region comprises FcMut193. In an embodiment, the Fe region comprises FcMut194. In an embodiment, the Fe region comprises FcMut195. In an embodiment, the Fe region comprises FcMut196. In an embodiment, the Fe region comprises FcMut197. In an embodiment, the Fe region comprises FcMut198. In an embodiment, the Fe region comprises FcMut199. In an embodiment, the Fe region comprises FcMut200. In an embodiment, the Fe region comprises FcMut201. In an embodiment, the Fe region comprises FcMut202. In an embodiment, the Fe region comprises FcMut203. In an embodiment, the Fe region comprises FcMut204. In an embodiment, the Fe region comprises FcMut205. In an embodiment, the Fe region comprises FcMut206. In an embodiment, the Fe region comprises FcMut207. In an embodiment, the Fe region comprises FcMut208. In an embodiment, the Fe region comprises FcMut209. In an embodiment, the Fe region comprises FcMut210. In an embodiment, the Fe region comprises FcMut2l 1. In an embodiment, the Fe region comprises FcMut212. In an embodiment, the Fe region comprises FcMut213. In an embodiment, the Fe region comprises FcMut214. In an embodiment, the Fe region comprises FcMut215. In an embodiment, the Fe region comprises FcMut216. In an embodiment, the Fe region comprises FcMut217. In an embodiment, the Fe region comprises FcMut218. In an embodiment, the Fe region comprises FcMut219. In an embodiment, the Fe region comprises FcMut220. In an embodiment, the Fe region comprises FcMut221. In an embodiment, the Fe region comprises FcMut222. In an embodiment, the Fe region comprises FcMut223. In an embodiment, the Fe region comprises FcMut224. In an embodiment, the Fe region comprises FcMut225. In an embodiment, the Fe region comprises FcMut226. In an embodiment, the Fe region comprises FcMut227. In an embodiment, the Fe region comprises FcMut228. In an embodiment, the Fe region comprises FcMut229. In an embodiment, the Fe region comprises FcMut230. In an embodiment, the Fe region comprises FcMut231. In an embodiment, the Fe region comprises FcMut232. In an embodiment, the Fe region comprises FcMut233. In an embodiment, the Fe region comprises FcMut234. In an embodiment, the Fe region comprises FcMut242. In an embodiment, the Fe region comprises FcMut243. In an embodiment, the Fe region comprises FcMut244.

Other exemplary Fe mutations are described, e.g., in International Application Publication No. WO2018/052556, US Patent Application Publication No. US2018/0037634, and Booth et al., MAbs. 2018; 10(7):1098-1110, the contents of which are incorporated by reference in their entirety.

In an embodiment, the Fe region is altered to extend half-life. For example, the Fe region can contain one or more of: FcMut183 (T256D-Q311V-A378V), FcMut197 (H285N-T307Q-N315D), FcMut213 (H285D-T307Q-A378V), FcMut215 (T307Q-Q31 1V-A378V), or FcMut228 (T256D-N286D-T307R-Q311V-A378V) (all according to EU numbering).

In an embodiment, the Fe region is altered to enhance ADCC. For example, the Fe region can contain one or more of: A330L-I332E-S239D, F243L-R292P-Y300L-V305I-P396L, or S298A-E333A-K334A. In an embodiment, afucosylation can be achieved by expression in a cell line such as CHO in which fucosyltransferase (FucT8) is knocked out.

In an embodiment, the Fe region is altered to enhance CDC. For example, the Fe region contains S267E-H268F-S324T.

In an embodiment, the Fe region is altered to enhance antibody-dependent cellular phagocytosis (ADCP). For example, the Fc region contains S239D-1332E-A330L.

In an embodiment, the only amino acids in the antibody molecule are canonical amino acids. In an embodiment, the antibody molecule comprises naturally-occurring amino acids; analogs, derivatives and congeners thereof; amino acid analogs having variant side chains; and/or all stereoisomers of any of any of the foregoing. The antibody molecule may comprise the D- or L-optical isomers of amino acids and peptidomimetics.

A polypeptide of an antibody molecule described herein may be linear or branched, it may comprise modified amino acids, and it may be interrupted by non-amino acids. The antibody molecule may also be modified; for example, by disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The antibody molecule described herein can be used alone in unconjugated form, or can be bound to a substance, e.g., a toxin or moiety (e.g., a therapeutic drug; a compound emitting radiation; molecules of plant, fungal, or bacterial origin; or a biological protein (e.g., a protein toxin) or particle (e.g., a recombinant viral particle, e.g., via a viral coat protein). For example, the anti-APRIL antibody can be coupled to a radioactive isotope such as an a-, 3-, or y-emitter, or a R-and y-emitter.

An antibody molecule can be derivatized or linked to another functional molecule (e.g., another peptide or protein). As used herein, a “derivatized” antibody molecule is one that has been modified.

Methods of derivatization include but are not limited to the addition of a fluorescent moiety, a radionucleotide, a toxin, an enzyme or an affinity ligand such as biotin. Accordingly, the antibody molecules are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a toxin, a pharmaceutical agent, and/or a protein or peptide that can mediate association of the antibody or antibody portion with another molecule (such as a streptavidin core region or a polyhistidine tag).

Some types of derivatized antibody molecule are produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an anti-dengue antibody molecule may be derivatized (or labeled) to include fluorescent compounds, various enzymes, prosthetic groups, luminescent materials, bioluminescent materials, fluorescent emitting metal atoms, e.g., europium (Eu), and other anthanides, and radioactive materials (described below). Exemplary fluorescent detectable agents include fluorescein, fluorescein isothiocyanate, rhodamine, 5dimethylamine-1-napthalenesulfonyl chloride, phycoerythrin and the like. An antibody may also be derivatized with detectable enzymes, such as alkaline phosphatase, horseradish peroxidase, P-galactosidase, acetylcholinesterase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody molecule may also be derivatized with a prosthetic group (e.g., streptavidin/biotin and avidin/biotin). For example, an antibody may be derivatized with biotin, and detected through indirect measurement of avidin or streptavidin binding.

Examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of bioluminescent materials include luciferase, luciferin, and aequorin.

Labeled antibody molecules can be used, for example, diagnostically and/or experimentally in a number of contexts, including (i) to isolate a predetermined antigen by standard techniques, such as affinity chromatography or immunoprecipitation; (ii) to detect a predetermined antigen (e.g., in a cellular lysate or cell supernatant) in order to evaluate the abundance and pattern of expression of the protein; (iii) to monitor protein levels in tissue as part of a clinical testing procedure, e.g., to determine the efficacy of a given treatment regimen.

An antibody molecule may be conjugated to another molecular entity, typically a label or a therapeutic (e.g., antimicrobial (e.g., antibacterial or bactericidal), immunomodulatory, immunostimularoty, cytotoxic, or cytostatic) agent or moiety. Radioactive isotopes can be used in diagnostic or therapeutic applications. Radioactive isotopes that can be coupled to the antibody molecules include, but are not limited to α-, β-, or γ-emitters, or β-and γ-emitters. Such radioactive isotopes include, but are not limited to iodine (¹³¹I or ¹²⁵ yttrum (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹ mTc), phosphorus (³²P), rhodium (¹⁸⁸Rh), sulfur (³⁵S), carbon (⁴C), tritium (3H), chromium (⁵¹Cr), chlorine (³⁶C1), cobalt (⁵⁷Co or ⁵⁸Co), iron (⁵⁹Fe), selenium (⁷⁵Se), or gallium (⁶⁷Ga).

Radioisotopes useful as therapeutic agents include yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), and rhodium (¹⁸⁸Rh). Radioisotopes useful as labels, e.g., for use in diagnostics, include iodine (¹³¹I or ¹²⁵1), indium (¹¹¹In), technetium (⁹⁹mTc), phosphorus (³²P), carbon (⁴C), and tritium (3H), or one or more of the therapeutic isotopes listed above.

The present disclosure provides radiolabeled antibody molecules and methods of labeling the same. In an embodiment, a method of labeling an antibody molecule is disclosed. The method includes contacting an antibody molecule, with a chelating agent, to thereby produce a conjugated antibody. The conjugated antibody is radiolabeled with a radioisotope, e.g., ¹¹¹Indium, ⁹⁰Yttrium and ¹⁷⁷Lutetium, to thereby produce a labeled antibody molecule.

In some aspects, this disclosure provides a method of making an antibody molecule disclosed herein. The method includes: providing an antigen, e.g., APRIL or a fragment thereof, obtaining an antibody molecule that specifically binds to the antigen; evaluating efficacy of the antibody molecule in modulating activity of the antigen and/or organism expressing the antigen, e.g., APRIL. The method can further include administering the antibody molecule, including a derivative thereof (e.g., a humanized antibody molecule) to a subject, e.g., a human.

This disclosure provides an isolated nucleic acid molecule encoding the above antibody molecule, vectors and host cells thereof. The nucleic acid molecule includes, but is not limited to, RNA, genomic DNA and cDNA.

Amino acid and nucleotide sequences of exemplary antibody molecules are described in Tables 1 and 2, respectively. Amino acid sequences of additional exemplary humanized antibody molecules are described in Table 5.

TABLE 1
The amino acid sequences of the heavy chain variable region (VH) and light chain
variable region (VL) of the exemplary anti-APRIL antibodies are provided as
follows. CDRs, defined according to the Kabat system, are underlined and bold,
while CDRs defined according to the Chothia system are italicized.

| | | | SEQ | | | SEQ | | | SEQ |
|---|---|---|---|---|---|---|---|---|---|
| | | | ID | Chothia | | ID | Kabat | | ID |
| Antibody | Chain | Amino Acid Sequence | NO | CDR | | NO | CDR | | NO |
| 2218 | VH | DVQLQESGPGLVKPSQSLSLTCSVTGYSITSGYYW | 9 | HCDR1 | GYSITSGY | 1 | HCDR1 | SGYYWN | 7 |
| | | NWIRQFPGNKLEWMGYISYDGYNNYNPSLKNRISI | | HCDR2 | SYDGY | 2 | HCDR2 | YISYDGYNNYN | 8 |
| | | TRDTSKNQFFLKLNSVTTEDTATYYCANYYDYEDW | | | | | | PSLKN | |
| | | YFGVWGTGTTVTVSS | | HCDR3 | YYDYEDWYFGV | 3 | HCDR3 | YYDYEDWYFGV | 3 |
| | VL | DIVLTQSPASLAMSLGKRATISCRASESVSIIGTN | 10 | LCDR1 | RASESVSIIGTNSIH | 4 | LCDR1 | RASESVSIIGT | 4 |
| | | SIHWYQQKPGQPPKLLIYHASNLETGVPARFSGSG | | | | | | NSIH | |
| | | SRTDFTLTIDPVEEDDVAIYYCLQSRKIPYTFGGG | | LCDR2 | HASNLET | 5 | LCDR2 | HASNLET | 5 |
| | | TKLEIK | | LCDR3 | LQSRKIPYT | 6 | LCDR3 | LQSRKIPYT | 6 |
| 2419 | VH | QVQLQQSGAELVKPGASVRLSCEASGYTFTDYTIH | 19 | HCDR1 | GYTFTDY | 11 | HCDR1 | DYTIH | 17 |
| | | WVKQRSGQGLEWIGWIYPLRGSINYNEKFKDKATL | | HCDR2 | YPLRGS | 12 | HCDR2 | WIYPLRGSINY | 18 |
| | | TADKSSSTVYLELGRLTSKDSAVYFCARHGAYYSN | | | | | | NEKFKD | |
| | | AFDYWGQGTTLTVSS | | HCDR3 | HGAYYSNAFDY | 13 | HCDR3 | HGAYYSNAFDY | 13 |
| | VL | NIVMTQSPASLAVSLGQRATISCRASESVDNDGIR | 20 | LCDR1 | RASESVDNDGIRFMH | 14 | LCDR1 | RASESVDNDGI | 14 |
| | | FMHWYQQKPGQPPKLLIYRASNLESGIPARFSGSG | | | | | | RFMH | |
| | | SRTDFTLTINPVETDDVATYYCQQSNKDPYTFGGG | | LCDR2 | RASNLES | 15 | LCDR2 | RASNLES | 15 |
| | | TKLELK | | LCDR3 | QQSNKDPYT | 16 | LCDR3 | QQSNKDPYT | 16 |
| 2419-1305 | VH | QVQLVQSGAEVKKPGASVKVSCKASGYTFTDYTIH | 283 | HCDR1 | GYTFTDY | 11 | HCDR1 | DYTIH | 17 |
| | | WVRQATGQGLEWMGWIYPLRGSINYAQKFQGRVTM | | HCDR2 | YPLRGS | 12 | HCDR2 | WIYPLRGSINY | 282 |
| | | TANKSISTVYMELSSLRSEDTAVYFCARHGAYYSN | | | | | | AQKFQG | |
| | | AFDYWGQGTLVTVSS | | HCDR3 | HGAYYSNAFDY | 13 | HCDR3 | HGAYYSNAFDY | 13 |
| | VL | EIVMTQSPATLSVSPGERATLSCRASESVDNDGIR | 284 | LCDR1 | RASESVDNDGIRFLH | 280 | LCDR1 | RASESVDNDGI | 280 |
| | | FLHWYQQKPGQAPRLLIYRASNRETGIPARFSGSG | | | | | | RFLH | |
| | | SGTEFTLTISSLQSEDFAVYYCQQSNKDPYTFGGG | | LCDR2 | RASNRET | 281 | LCDR2 | RASNRET | 281 |
| | | TKVEIK | | LCDR3 | QQSNKDPYT | 16 | LCDR3 | QQSNKDPYT | 16 |
| 2419-1306 | VH | QVQLVQSGAEVKKPGASVKVSCKASGYTFTDYTIH | 283 | HCDR1 | GYTFTDY | 11 | HCDR1 | DYTIH | 17 |
| | | WVRQATGQGLEWMGWIYPLRGSINYAQKFQGRVTM | | HCDR2 | YPLRGS | 12 | HCDR2 | WIYPLRGSINY | 282 |
| | | TANKSISTVYMELSSLRSEDTAVYFCARHGAYYSN | | | | | | AQKFQG | |
| | | AFDYWGQGTLVTVSS | | HCDR3 | HGAYYSNAFDY | 13 | HCDR3 | HGAYYSNAFDY | 13 |
| | VL | EIVMTQSPATLSVSPGERATLSCRASESVDNDGIR | 286 | LCDR1 | RASESVDNDGIRFLH | 280 | LCDR1 | RASESVDNDGI | 280 |
| | | FLHWYQQKPGQAPRLLIYRASTRATGIPARFSGSG | | | | | | RFLH | |
| | | SRTEFTLTISSLQSEDFAVYYCQQSNKDPYTFGGG | | LCDR2 | RASTRAT | 285 | LCDR2 | RASTRAT | 285 |
| | | TKVEIK | | LCDR3 | QQSNKDPYT | 16 | LCDR3 | QQSNKDPYT | 16 |
| 2419-1310 | VH | QVQLVQSGAEVKKPGASVKVSCKASGYTFTDYTIH | 283 | HCDR1 | GYTFTDY | 11 | HCDR1 | DYTIH | 17 |
| | | WVRQATGQGLEWMGWIYPLRGSINYAQKFQGRVTM | | HCDR2 | YPLRGS | 12 | HCDR2 | WIYPLRGSINY | 282 |
| | | TANKSISTVYMELSSLRSEDTAVYFCARHGAYYSN | | | | | | AQKFQG | |
| | | AFDYWGQGTLVTVSS | | HCDR3 | HGAYYSNAFDY | 13 | HCDR3 | HGAYYSNAFDY | 13 |
| | VL | DIVMTQSPDSLAVSLGERATINCKSSQSVDNDGIR | 316 | LCDR1 | KSSQSVDNDGIRFLH | 314 | LCDR1 | KSSQSVDNDGI | 314 |
| | | FLHWYQQKPGQPPKLLIYRASTRESGVPDRFSGSG | | | | | | RFLH | |
| | | SGTDFTLTISSLQAEDVAVYYCQQSNKDPYTFGGG | | LCDR2 | RASTRES | 315 | LCDR2 | RASTRES | 315 |
| | | TKVEIK | | LCDR3 | QQSNKDPYT | 16 | LCDR3 | QQSNKDPYT | 16 |
| 2419-0806 | VH | EVQLVQSGAEVKKPGESLKISCKASGYTFTDYTIH | 288 | HCDR1 | GYTFTDY | 11 | HCDR1 | DYTIH | 17 |
| | | WVRQMPGKGLEWMGWIYPLRGSINYSPSFQGQVTI | | HCDR2 | YPLRGS | 12 | HCDR2 | WIYPLRGSINY | 287 |
| | | SADKSISTVYLOWSSLKASDTAMYFCARHGAYYSN | | | | | | SPSFQG | |
| | | AFDYWGQGTLVTVSS | | HCDR3 | HGAYYSNAFDY | 13 | HCDR3 | HGAYYSNAFDY | 13 |
| | VL | EIVMTQSPATLSVSPGERATLSCRASESVDNDGIR | 286 | LCDR1 | RASESVDNDGIRFLH | 280 | LCDR1 | RASESVDNDGI | 280 |
| | | FLHWYQQKPGQAPRLLIYRASTRATGIPARFSGSG | | | | | | RFLH | |
| | | SRTEFTLTISSLQSEDFAVYYCQQSNKDPYTFGGG | | LCDR2 | RASTRAT | 285 | LCDR2 | RASTRAT | 285 |
| | | TKVEIK | | LCDR3 | QQSNKDPYT | 16 | LCDR3 | QQSNKDPYT | 16 |
| 2419-0205 | VH | QVQLVQSGAEVKKPGSSVKVSCKASGYTFTDYTIH | 289 | HCDR1 | GYTFTDY | 11 | HCDR1 | DYTIH | 17 |
| | | WVRQAPGQGLEWMGWIYPLRGSINYAQKFQGRVTI | | HCDR2 | YPLRGS | 12 | HCDR2 | WIYPLRGSINY | 282 |
| | | TADKSTSTAYMELSSLRSEDTAVYFCARHGAYYSN | | | | | | AQKFQG | |
| | | AFDYWGQGTLVTVSS | | HCDR3 | HGAYYSNAFDY | 13 | HCDR3 | HGAYYSNAFDY | 13 |
| | VL | EIVMTQSPATLSVSPGERATLSCRASESVDNDGIR | 284 | LCDR1 | RASESVDNDGIRFLH | 280 | LCDR1 | RASESVDNDGI | 280 |
| | | FLHWYQQKPGQAPRLLIYRASNRETGIPARFSGSG | | | | | | RFLH | |
| | | SGTEFTLTISSLQSEDFAVYYCQQSNKDPYTFGGG | | LCDR2 | RASNRET | 281 | LCDR2 | RASNRET | 281 |
| | | TKVEIK | | LCDR3 | QQSNKDPYT | 16 | LCDR3 | QQSNKDPYT | 16 |
| 2419-0406 | VH | QVQLVQSGAEVKKPGSSVKVSCKASGYTFTDYTIH | 291 | HCDR1 | GYTFTDY | 11 | HCDR1 | DYTIH | 17 |
| | | WVRQAPGQGLEWMGWIYPLRGSINYAEKFKGRVTL | | HCDR2 | YPLRGS | 12 | HCDR2 | WIYPLRGSINY | 290 |
| | | TADKSTSTVYMELSSLRSEDTAVYFCARHGAYYSN | | | | | | AEKFKG | |
| | | AFDYWGQGTLVTVSS | | HCDR3 | HGAYYSNAFDY | 13 | HCDR3 | HGAYYSNAFDY | 13 |
| | VL | EIVMTQSPATLSVSPGERATLSCRASESVDNDGIR | 286 | LCDR1 | RASESVDNDGIRFLH | 280 | LCDR1 | RASESVDNDGI | 280 |
| | | FLHWYQQKPGQAPRLLIYRASTRATGIPARFSGSG | | | | | | RFLH | |
| | | SRTEFTLTISSLQSEDFAVYYCQQSNKDPYTFGGG | | LCDR2 | RASTRAT | 285 | LCDR2 | RASTRAT | 285 |
| | | TKVEIK | | LCDR3 | QQSNKDPYT | 16 | LCDR3 | QQSNKDPYT | 16 |
| 2419-0605 | VH | QVQLVQSGAEVKKPGASVKVSCKASGYTFTDYTIH | 317 | HCDR1 | GYTFTDY | 11 | HCDR1 | DYTIH | 17 |
| | | WVRQAPGQGLEWMGWIYPLRGSINYAQKFQGRVTL | | HCDR2 | YPLRGS | 12 | HCDR2 | WIYPLRGSINY | 282 |
| | | TADKSTSTVYMELSSLRSEDTAVYFCARHGAYYSN | | | | | | AQKFQG | |
| | | AFDYWGQGTLVTVSS | | HCDR3 | HGAYYSNAFDY | 13 | HCDR3 | HGAYYSNAFDY | 13 |
| | VL | EIVMTQSPATLSVSPGERATLSCRASESVDNDGIR | 284 | LCDR1 | RASESVDNDGIRFLH | 280 | LCDR1 | RASESVDNDGI | 280 |
| | | FLHWYQQKPGQAPRLLIYRASNRETGIPARFSGSG | | | | | | RFLH | |
| | | SGTEFTLTISSLQSEDFAVYYCQQSNKDPYTFGGG | | LCDR2 | RASNRET | 281 | LCDR2 | RASNRET | 281 |
| | | TKVEIK | | LCDR3 | QQSNKDPYT | 16 | LCDR3 | QQSNKDPYT | 16 |
| 2419-0805 | VH | EVQLVQSGAEVKKPGESLKISCKASGYTFTDYTIH | 288 | HCDR1 | GYTFTDY | 11 | HCDR1 | DYTIH | 17 |
| | | WVRQMPGKGLEWMGWIYPLRGSINYSPSFQGQVTI | | HCDR2 | YPLRGS | 12 | HCDR2 | WIYPLRGSINY | 287 |
| | | SADKSISTVYLQWSSLKASDTAMYFCARHGAYYSN | | | | | | SPSFQG | |
| | | AFDYWGQGTLVTVSS | | HCDR3 | HGAYYSNAFDY | 13 | HCDR3 | HGAYYSNAFDY | 13 |
| | VL | EIVMTQSPATLSVSPGERATLSCRASESVDNDGIR | 284 | LCDR1 | RASESVDNDGIRFLH | 280 | LCDR1 | RASESVDNDGI | 280 |
| | | FLHWYQQKPGQAPRLLIYRASNRETGIPARFSGSG | | | | | | RFLH | |
| | | SGTEFTLTISSLQSEDFAVYYCQQSNKDPYTFGGG | | LCDR2 | RASNRET | 281 | LCDR2 | RASNRET | 281 |
| | | TKVEIK | | LCDR3 | QQSNKDPYT | 16 | LCDR3 | QQSNKDPYT | 16 |
| 2419-0105 | VH | QVQLVQSGAEVKKPGASVKVSCKASGYTFTDYTIH | 292 | HCDR1 | GYTFTDY | 11 | HCDR1 | DYTIH | 17 |
| | | WVRQAPGQGLEWMGWIYPLRGSINYAQKFQGRVTM | | HCDR2 | YPLRGS | 12 | HCDR2 | WIYPLRGSINY | 282 |
| | | TADKSISTVYMELSRLRSDDTAVYYCARHGAYYSN | | | | | | AQKFQG | |
| | | AFDYWGQGTLVTVSS | | HCDR3 | HGAYYSNAFDY | 13 | HCDR3 | HGAYYSNAFDY | 13 |
| | VL | EIVMTQSPATLSVSPGERATLSCRASESVDNDGIR | 284 | LCDR1 | RASESVDNDGIRFLH | 280 | LCDR1 | RASESVDNDGI | 280 |
| | | FLHWYQQKPGQAPRLLIYRASNRETGIPARFSGSG | | | | | | RFLH | |
| | | SGTEFTLTISSLQSEDFAVYYCQQSNKDPYTFGGG | | LCDR2 | RASNRET | 281 | LCDR2 | RASNRET | 281 |
| | | TKVEIK | | LCDR3 | QQSNKDPYT | 16 | LCDR3 | QQSNKDPYT | 16 |
| 2419-1204 | VH | QVQLVQSGAEVKKPGASVKVSCKASGYTFTDYTIH | 294 | HCDR1 | GYTFTDY | 11 | HCDR1 | DYTIH | 17 |
| | | WVRQATGQGLEWMGWIYPLRGSINYAQKFQGRVTM | | HCDR2 | YPLRGS | 12 | HCDR2 | WIYPLRGSINY | 282 |
| | | TANKSSSTVYMELSSLRSEDTAVYFCARHGAYYSN | | | | | | AQKFQG | |
| | | AFDYWGQGTLVTVSS | | HCDR3 | HGAYYSNAFDY | 13 | HCDR3 | HGAYYSNAFDY | 13 |
| | VL | EIVMTQSPATLSVSPGERATLSCRASESVDNDGIR | 295 | LCDR1 | RASESVDNDGIRFLH | 280 | LCDR1 | RASESVDNDGI | 280 |
| | | FLHWYQQKPGQAPRLLIYRASTLETGIPARFSGSG | | | | | | RFLH | |
| | | SGTEFTLTISSLQSEDFAVYYCQQSNKDPYTFGGG | | LCDR2 | RASTLET | 293 | LCDR2 | RASTLET | 293 |
| | | TKVEIK | | LCDR3 | QQSNKDPYT | 16 | LCDR3 | QQSNKDPYT | 16 |
| 2419-1205 | VH | QVQLVQSGAEVKKPGASVKVSCKASGYTFTDYTIH | 294 | HCDR1 | GYTFTDY | 11 | HCDR1 | DYTIH | 17 |
| | | WVRQATGQGLEWMGWIYPLRGSINYAQKFQGRVTM | | HCDR2 | YPLRGS | 12 | HCDR2 | WIYPLRGSINY | 282 |
| | | TANKSSSTVYMELSSLRSEDTAVYFCARHGAYYSN | | | | | | AQKFQG | |
| | | AFDYWGQGTLVTVSS | | HCDR3 | HGAYYSNAFDY | 13 | HCDR3 | HGAYYSNAFDY | 13 |
| | VL | EIVMTQSPATLSVSPGERATLSCRASESVDNDGIR | 284 | LCDR1 | RASESVDNDGIRFLH | 280 | LCDR1 | RASESVDNDGI | 280 |
| | | FLHWYQQKPGQAPRLLIYRASNRETGIPARFSGSG | | | | | | RFLH | |
| | | SGTEFTLTISSLQSEDFAVYYCQQSNKDPYTFGGG | | LCDR2 | RASNRET | 281 | LCDR2 | RASNRET | 281 |
| | | TKVEIK | | LCDR3 | QQSNKDPYT | 16 | LCDR3 | QQSNKDPYT | 16 |
| 2419-1210 | VH | QVQLVQSGAEVKKPGASVKVSCKASGYTFTDYTIH | 294 | HCDR1 | GYTFTDY | 11 | HCDR1 | DYTIH | 17 |
| | | WVRQATGQGLEWMGWIYPLRGSINYAQKFQGRVTM | | HCDR2 | YPLRGS | 12 | HCDR2 | WIYPLRGSINY | 282 |
| | | TANKSSSTVYMELSSLRSEDTAVYFCARHGAYYSN | | | | | | AQKFQG | |
| | | AFDYWGQGTLVTVSS | | HCDR3 | HGAYYSNAFDY | 13 | HCDR3 | HGAYYSNAFDY | 13 |
| | VL | DIVMTQSPDSLAVSLGERATINCKSSQSVDNDGIR | 316 | LCDR1 | KSSQSVDNDGIRFLH | 314 | LCDR1 | KSSQSVDNDGI | 314 |
| | | FLHWYQQKPGQPPKLLIYRASTRESGVPDRFSGSG | | | | | | RFLH | |
| | | SGTDFTLTISSLQAEDVAVYYCQQSNKDPYTFGGG | | LCDR2 | RASTRES | 315 | LCDR2 | RASTRES | 315 |
| | | TKVEIK | | LCDR3 | QQSNKDPYT | 16 | LCDR3 | QQSNKDPYT | 16 |
| 2419-1406 | VH | QVQLVQSGAEVKKPGASVKVSCKASGYTFTDYTIH | 296 | HCDR1 | GYTFTDY | 11 | HCDR1 | DYTIH | 17 |
| | | WVRQATGQGLEWMGWIYPLRGSINYAQKFQGRVTM | | HCDR2 | YPLRGS | 12 | HCDR2 | WIYPLRGSINY | 282 |
| | | TADKSISTVYMELSSLRSEDTAVYFCARHGAYYSN | | | | | | AQKFQG | |
| | | AFDYWGQGTLVTVSS | | HCDR3 | HGAYYSNAFDY | 13 | HCDR3 | HGAYYSNAFDY | 13 |
| | VL | EIVMTQSPATLSVSPGERATLSCRASESVDNDGIR | 286 | LCDR1 | RASESVDNDGIRFLH | 280 | LCDR1 | RASESVDNDGI | 280 |
| | | FLHWYQQKPGQAPRLLIYRASTRATGIPARFSGSG | | | | | | RFLH | |
| | | SRTEFTLTISSLQSEDFAVYYCQQSNKDPYTFGGG | | LCDR2 | RASTRAT | 285 | LCDR2 | RASTRAT | 285 |
| | | TKVEIK | | LCDR3 | QQSNKDPYT | 16 | LCDR3 | QQSNKDPYT | 16 |
| 2419-0206 | VH | QVQLVQSGAEVKKPGSSVKVSCKASGYTFTDYTIH | 289 | HCDR1 | GYTFTDY | 11 | HCDR1 | DYTIH | 17 |
| | | WVRQAPGQGLEWMGWIYPLRGSINYAQKFQGRVTI | | HCDR2 | YPLRGS | 12 | HCDR2 | WIYPLRGSINY | 282 |
| | | TADKSTSTAYMELSSLRSEDTAVYFCARHGAYYSN | | | | | | AQKFQG | |
| | | AFDYWGQGTLVTVSS | | HCDR3 | HGAYYSNAFDY | 13 | HCDR3 | HGAYYSNAFDY | 13 |
| | VL | EIVMTQSPATLSVSPGERATLSCRASESVDNDGIR | 286 | LCDR1 | RASESVDNDGIRFLH | 280 | LCDR1 | RASESVDNDGI | 280 |
| | | FLHWYQQKPGQAPRLLIYRASTRATGIPARFSGSG | | | | | | RFLH | |
| | | SRTEFTLTISSLQSEDFAVYYCQQSNKDPYTFGGG | | LCDR2 | RASTRAT | 285 | LCDR2 | RASTRAT | 285 |
| | | TKVEIK | | LCDR3 | QQSNKDPYT | 16 | LCDR3 | QQSNKDPYT | 16 |
| 2621 | VH | EVQLQQSGAELVRPGSSVKMSCKTSGYTFTSYGIN | 29 | HCDR1 | GYTFTSY | 21 | HCDR1 | SYGIN | 27 |
| | | WVKQRPGQGLEWIGYIYIGNGYAEYNERFKGKATL | | HCDR2 | YIGNGY | 22 | HCDR2 | YIYIGNGYAEY | 28 |
| | | TSDTSSSTAYMQLSSLTSEDSAIYFCALYYPWFTY | | | | | | NERFKG | |
| | | WGQGTLVTVSA | | HCDR3 | YYPWFTY | 23 | HCDR3 | YYPWFTY | 23 |
| | VL | DIQMTQSPASLSASVGDSVTITCRASENIYSYLAW | 30 | LCDR1 | RASENIYSYLA | 24 | LCDR1 | RASENIYSYLA | 24 |
| | | YQQKQGKSPQLLVYNAKTLAEGVPSRFSGSGSGTQ | | LCDR2 | NAKTLAE | 25 | LCDR2 | NAKTLAE | 25 |
| | | FSLKINSLQPEDFGNYYCQHHYDTPFTFGGGTKLE | | LCDR3 | QHHYDTPFT | 26 | LCDR3 | QHHYDTPFT | 26 |
| | | IK | | | | | | | |
| 2922 | VH | QVQLHQSGPELVKPGASVKLSCKTSGYTFTSYDVF | 39 | HCDR1 | GYTFTSY | 21 | HCDR1 | SYDVF | 37 |
| | | WVKQRPGQGLEWIGWIYPRDSSTKYNEKFKGKATL | | HCDR2 | YPRDSS | 32 | HCDR2 | WIYPRDSSTKY | 38 |
| | | TVDTSSSTAYMELHSLTSEDSAVYFCAKEGYDYDK | | | | | | NEKFKG | |
| | | RGFDYWGQGTTLTVSS | | HCDR3 | EGYDYDKRGFDY | 33 | HCDR3 | EGYDYDKRGFD | 33 |
| | | | | | | | | Y | |
| | VL | DIVLTQSPASLAVSLGQRAIISCKASQSVSFAGTN | 40 | LCDR1 | KASQSVSFAGTNLMH | 34 | LCDR1 | KASQSVSFAGT | 34 |
| | | LMHWYQQRPGQQPKLLIYRASNLEPGVPTRFSGSG | | | | | | NLMH | |
| | | SRTDFTLNIHPVEEDDAATYYCQQSREYPWTFGGG | | LCDR2 | RASNLEP | 35 | LCDR2 | RASNLEP | 35 |
| | | TKLEIK | | LCDR3 | QQSREYPWT | 36 | LCDR3 | QQSREYPWT | 36 |
| 3125 | VH | QVQLQQSGAELVRPGASVTLSCKASGYTFTDYEMH | 49 | HCDR1 | GYTFTDY | 11 | HCDR1 | DYEMH | 47 |
| | | WVKQTPVHGLEWIGAIDPETGGTAYNQRFKGKAIL | | HCDR2 | DPETGG | 42 | HCDR2 | AIDPETGGTAY | 48 |
| | | TTDKSSITAYMELRSLTSEDSAVYYCTRWNDGDYW | | | | | | NQRFKG | |
| | | GQGTTLTVSS | | HCDR3 | WNDGDY | 43 | HCDR3 | WNDGDY | 43 |
| | VL | DVVMTQTPLSLSVTIGQPASISCKSSQSLLYSNGK | 50 | LCDR1 | KSSQSLLYSNGKTYLN | 44 | LCDR1 | KSSQSLLYSNG | 44 |
| | | TYLNWFQQRPGQSPKRLMYQVSKLDPGIPDRFSGS | | | | | | KTYLN | |
| | | GSETDFTLKISRVEAEDLGLYYCLQGTYYPYTFGG | | LCDR2 | QVSKLDP | 45 | LCDR2 | QVSKLDP | 45 |
| | | GTKLEIK | | LCDR3 | LOGTYYPYT | 46 | LCDR3 | LOGTYYPYT | 46 |
| 3327 | VH | EVQLQQSGPELVKPGASVKMSCKASGYSFTGYFMN | 59 | HCDR1 | GYSFTGY | 51 | HCDR1 | GYFMN | 57 |
| | | WVKQSHGKSLEWIGRINPYNGDTFYNQKFKGKATL | | HCDR2 | NPYNGD | 52 | HCDR2 | RINPYNGDTFY | 58 |
| | | TVDKSSSTAHMELRSLTSEDSALYYCASEGDGYYW | | | | | | NQKFKG | |
| | | YFDVWGAGTTVTVSS | | HCDR3 | EGDGYYWYFDV | 53 | HCDR3 | EGDGYYWYFDV | 53 |
| | VL | DIVLTQSPASLAVSLGORATISCRASESVDNYGIS | 60 | LCDR1 | RASESVDNYGISFMN | 54 | LCDR1 | RASESVDNYGI | 54 |
| | | FMNWFQQKPGQPPKLLIYAASNQGSGVPARFSGSG | | | | | | SFMN | |
| | | SGTDFSLNIHPMEEDDTAMYFCQQSKEVPRTFGGG | | LCDR2 | AASNQGS | 55 | LCDR2 | AASNOGS | 55 |
| | | TKLEIK | | LCDR3 | QQSKEVPRT | 56 | LCDR3 | QQSKEVPRT | 56 |
| 3525 | VH | QVQLQQSGAELVRPGASVKLSCKASGYTFTDHEMH | 66 | HCDR1 | GYTFTDH | 61 | HCDR1 | DHEMH | 64 |
| | | WVRQTPVHGLEWIGVIDPDTGDTTYNQKFKGKATL | | HCDR2 | DPDTGD | 62 | HCDR2 | VIDPDTGDTTY | 65 |
| | | TADKSSSTAYMDLRSLTSEDSAVFYCTRWTGGDYW | | | | | | NQKFKG | |
| | | GHGTTLTVSS | | HCDR3 | WTGGDY | 63 | HCDR3 | WTGGDY | 63 |
| | VL | DVVMTQTPLSLSVTIGQPASISCKSSQSLLYSNGK | 50 | LCDR1 | KSSQSLLYSNGKTYLN | 44 | LCDR1 | KSSQSLLYSNG | 44 |
| | | TYLNWFQQRPGQSPKRLMYQVSKLDPGIPDRFSGS | | | | | | KTYLN | |
| | | GSETDFTLKISRVEAEDLGLYYCLQGTYYPYTFGG | | LCDR2 | QVSKLDP | 45 | LCDR2 | QVSKLDP | 45 |
| | | GTKLEIK | | LCDR3 | LQGTYYPYT | 46 | LCDR3 | LQGTYYPYT | 46 |
| 3530 | VH | QVQLQQSGAELVRPGASVKLSCKASGYTFTDHEMH | 66 | HCDR1 | GYTFTDH | 61 | HCDR1 | DHEMH | 64 |
| | | WVRQTPVHGLEWIGVIDPDTGDTTYNQKFKGKATL | | HCDR2 | DPDTGD | 62 | HCDR2 | VIDPDTGDTTY | 65 |
| | | TADKSSSTAYMDLRSLTSEDSAVFYCTRWTGGDYW | | | | | | NQKFKG | |
| | | GHGTTLTVSS | | HCDR3 | WTGGDY | 63 | HCDR3 | WTGGDY | 63 |
| | VL | DAVMTQTPLSLSVTIGQPASISCKSSQSLLYSDGK | 70 | LCDR1 | KSSQSLLYSDGKTYLN | 67 | LCDR1 | KSSQSLLYSDG | 67 |
| | | TYLNWFQQRPGQSPKRLMYQVSKLDPGIPDRFSGS | | | | | | KTYLN | |
| | | GSETDFTLKISRVEAEDLGVYYCLQGTYYPYTFGS | | LCDR2 | QVSKLDP | 45 | LCDR2 | QVSKLDP | 45 |
| | | GTKLEIK | | LCDR3 | LOGTYYPYT | 46 | LCDR3 | LOGTYYPYT | 46 |
| 4035 | VH | QVQLKESGPGLVAPSQSLSITCTVSGFSLTIYDVH | 101 | HCDR1 | GFSLTIY | 93 | HCDR1 | IYDVH | 99 |
| | | WVRQSPGKGLEWLGVIWSDGSTDYNAAFISRLSIS | | HCDR2 | WSDGS | 94 | HCDR2 | VIWSDGSTDYN | 100 |
| | | KDNSKSQVFFKMNSLQADDTAIYYCARNWVDQAWF | | | | | | AAFIS | |
| | | AYWGQGTLVTVSA | | HCDR3 | NWVDQAWFAY | 95 | HCDR3 | NWVDQAWFAY | 95 |
| | VL | DIQMTQSPASLSASVGETITITCRASKNIYSYLAW | 102 | LCDR1 | RASKNIYSYLA | 96 | LCDR1 | RASKNIYSYLA | 96 |
| | | YQQKQGKSPQLLVYNAKTLPEGVPSRFSGSGSGTQ | | LCDR2 | NAKTLPE | 97 | LCDR2 | NAKTLPE | 97 |
| | | FSLKINSLQPEDFGSYYCQHHYGTPLTFGAGTKLE | | LCDR3 | QHHYGTPLT | 98 | LCDR3 | QHHYGTPLT | 98 |
| | | LK | | | | | | | |
| 4035-062 | VH | QVQLQESGPGLVKPSETLSLTCTVSGFSLTIYDVH | 225 | HCDR1 | GFSLTIY | 93 | HCDR1 | IYDVH | 99 |
| | | WVRQPPGKGLEWIGVIWSDGSTDYNPSLKSRVTIS | | HCDR2 | WSDGS | 94 | HCDR2 | VIWSDGSTDYN | 273 |
| | | KDTSKNQVSLKLSSVTAADTAVYYCARNWVDQAWF | | | | | | PSLKS | |
| | | AYWGQGTLVTVSS | | HCDR3 | NWVDQAWFAY | 95 | HCDR3 | NWVDQAWFAY | 95 |
| | VL | DIQMTQSPSSLSASVGDRVTITCRASKNIYSYLAW | 229 | LCDR1 | RASKNIYSYLA | 96 | LCDR1 | RASKNIYSYLA | 96 |
| | | YQQKPGKAPKLLVYNAKTLPEGVPSRFSGSGSGTD | | LCDR2 | NAKTLPE | 97 | LCDR2 | NAKTLPE | 97 |
| | | FTLTISSLQPEDFATYYCQHHYGTPLTFGQGTKLE | | LCDR3 | QHHYGTPLT | 98 | LCDR3 | QHHYGTPLT | 98 |
| | | IK | | | | | | | |
| 3934 | VH | QVQLQQSGPELVKPGASVKLSCKAAGYIFTDYTIN | 111 | HCDR1 | GYIFTDY | 103 | HCDR1 | DYTIN | 109 |
| | | WVKQSPGQGLEWIGWIYPGSGNRKYNDKFKGKATM | | HCDR2 | YPGSGN | 104 | HCDR2 | WIYPGSGNRKY | 110 |
| | | TADKSSSTAYMQLSSLTSEDSAVYFCARESNYVGY | | | | | | NDKFKG | |
| | | YAMDYWGQGTSVTVSS | | HCDR3 | ESNYVGYYAMDY | 105 | HCDR3 | ESNYVGYYAMD | 105 |
| | | | | | | | | Y | |
| | VL | DVLMTQTPLSLPVSLGDQASISCRSSQSVVNSNGN | 112 | LCDR1 | RSSQSVVNSNGNTYLE | 106 | LCDR1 | RSSQSVVNSNG | 106 |
| | | TYLEWYLQKPGQSPNLLIYKVSNRFSGVPDRFSGS | | | | | | NTYLE | |
| | | GSGTDFTLKISRVEAEDLGVYYCFQGSHVPWTFGG | | LCDR2 | KVSNRFS | 107 | LCDR2 | KVSNRFS | 107 |
| | | GTKLEIK | | LCDR3 | FQGSHVPWT | 108 | LCDR3 | FQGSHVPWT | 108 |
| 3833 | VH | QVQLQQSGAELVRPGTSVKMSCKAAGYTFTNYWIG | 121 | HCDR1 | GYTFTNY | 113 | HCDR1 | NYWIG | 119 |
| | | WVKQRPGHGLEWIGDIYPGGIGGGYTKYNEKFKGK | | HCDR2 | YPGGIGGGY | 114 | HCDR2 | DIYPGGIGGGY | 120 |
| | | ATLTADTSSSTAYMQLGSLTSEDSAIYFCSRSETG | | | | | | TKYNEKFKG | |
| | | RAMDYWGQGTSVTVSS | | HCDR3 | SETGRAMDY | 115 | HCDR3 | SETGRAMDY | 115 |
| | VL | DIQMTQSPSSLSASLGGKVTITCKASQDINKYIAW | 122 | LCDR1 | KASQDINKYIA | 116 | LCDR1 | KASQDINKYIA | 116 |
| | | YQHKPGKGPRLLIHYTSTLKPGIPSRFSGSGSGRD | | LCDR2 | YTSTLKP | 117 | LCDR2 | YTSTLKP | 117 |
| | | YSFSISDLEPEDIATYYCLQYDNLNTFGGGTKLEI | | LCDR3 | LOYDNLNT | 118 | LCDR3 | LQYDNLNT | 118 |
| | | K | | | | | | | |
| 3631 | VH | EIQLQQSGPELVKPGASVKVSCKASGYSFTDYNIY | 131 | HCDR1 | GYSFTDY | 123 | HCDR1 | DYNIY | 129 |
| | | WVKQSHGKSLEWIGYIDPSNGGPGYNQKFRGKATL | | HCDR2 | DPSNGG | 124 | HCDR2 | YIDPSNGGPGY | 130 |
| | | TVDKSSSTAFLHLNSLTSEDSAVYYCARRDNYGSG | | | | | | NQKFRG | |
| | | TMDYWGQGTSVTVSS | | HCDR3 | RDNYGSGTMDY | 125 | HCDR3 | RDNYGSGTMDY | 125 |
| | VL | DIVMTQSQKFMSTSVGDRVSITCKASQNVGTDVSW | 132 | LCDR1 | KASQNVGTDVS | 126 | LCDR1 | KASQNVGTDVS | 126 |
| | | YQQKPGKSPKPLIYWASNRFTGVPDRFIGSGSGTD | | LCDR2 | WASNRFT | 127 | LCDR2 | WASNRFT | 127 |
| | | FTLTISNVQSEDLADYFCEQYSIYPLTFGAGTKLE | | LCDR3 | EQYSIYPLT | 128 | LCDR3 | EQYSIYPLT | 128 |
| | | LK | | | | | | | |
| 3732 | VH | EIQLQQSGPELVKPGASVKVSCKASGYSFTDDNMY | 140 | HCDR1 | GYSFTDD | 133 | HCDR1 | DDNMY | 138 |
| | | WVKQSHGKSLEWIGYIDPLNGGTGYNQKFKGKATL | | HCDR2 | DPLNGG | 134 | HCDR2 | YIDPLNGGTGY | 139 |
| | | TVDKSSSTAFLHLNSLTSEDSAVYYCARRDNYATG | | | | | | NQKFKG | |
| | | TMDYWGQGTSVTVSS | | HCDR3 | RDNYATGTMDY | 135 | HCDR3 | RDNYATGTMDY | 135 |
| | VL | DIVMTQSQKFMSTSVGDRVSITCKASKNVGTDVSW | 141 | LCDR1 | KASKNVGTDVS | 136 | LCDR1 | KASKNVGTDVS | 136 |
| | | YQQKPGKSPKPLIYWASNRFTGVPDRFTGSGSGTD | | LCDR2 | WASNRFT | 127 | LCDR2 | WASNRFT | 127 |
| | | FTLTINNVQSEDLADYFCEQYSSYPLTFGAGTKLE | | LCDR3 | EQYSSYPLT | 137 | LCDR3 | EQYSSYPLT | 137 |
| | | LK | | | | | | | |
| 4338 | VH | EVQLQQSGPELVKPGASVKISCKASGYTFTDYNMD | 151 | HCDR1 | GYTFTDY | 11 | HCDR1 | DYNMD | 149 |
| | | WVKQSHGKSLEWIGNIYPINGYTGYNQRFKNKATL | | HCDR2 | YPINGY | 142 | HCDR2 | NIYPINGYTGY | 150 |
| | | TVDKSSSTAYMELHSLTSEDSAVYYCARDSNYVGW | | | | | | NQRFKN | |
| | | YFDVWGAGTTVTVSS | | HCDR3 | DSNYVGWYFDV | 143 | HCDR3 | DSNYVGWYFDV | 143 |
| | VL | DVVMTQTPLSLPVSLGDQASISCRSSQSLVHSNGN | 152 | LCDR1 | RSSQSLVHSNGNTYLH | 144 | LCDR1 | RSSQSLVHSNG | 144 |
| | | TYLHWYLQKPGQSPKLLIYKVSNRFSGVPDRFSGS | | | | | | NTYLH | |
| | | GSGTDFTFKISRVEAEDLGVYFCSQSTHVPRTFGG | | LCDR2 | KVSNRFS | 107 | LCDR2 | KVSNRFS | 107 |
| | | GTKLEIK | | LCDR3 | SQSTHVPRT | 145 | LCDR3 | SQSTHVPRT | 145 |
| | VL | DIVLTQSPASLTVSLGQRATFSCRASKSVSTSGYS | 153 | LCDR1 | RASKSVSTSGYSYMH | 146 | LCDR1 | RASKSVSTSGY | 146 |
| | | YMHWYQQKPGQPPKLLIYFTSDLEPGVPARFTGSG | | | | | | SYMH | |
| | | SGTDFTLNIHPVEEEDAATYYCQHSRELPYPFGGG | | LCDR2 | FTSDLEP | 147 | LCDR2 | FTSDLEP | 147 |
| | | TKLEIK | | LCDR3 | QHSRELPYP | 148 | LCDR3 | QHSRELPYP | 148 |
| 4540 | VH | QVQLQQSGPELVKPGASVKISCKASGYTFADYYIN | 161 | HCDR1 | GYTFADY | 154 | HCDR1 | DYYIN | 159 |
| | | WVKQRPGQGLEWIGWIFPGSGSTYYNEKFKGKATL | | HCDR2 | FPGSGS | 155 | HCDR2 | WIFPGSGSTYY | 160 |
| | | TVDKSSSTAYMLLSSLTSEDSAVYFCARGDSGRAM | | | | | | NEKFKG | |
| | | DYWGQGTSVTVSS | | HCDR3 | GDSGRAMDY | 156 | HCDR3 | GDSGRAMDY | 156 |
| | VL | DIQMTQSPSSLSASLGGKVTITCKASQDINKYIAW | 162 | LCDR1 | KASQDINKYIA | 116 | LCDR1 | KASQDINKYIA | 116 |
| | | YQHKPGKGPRLLIHYTSTLQSGIPSRFSGSGSGRD | | LCDR2 | YTSTLQS | 157 | LCDR2 | YTSTLQS | 157 |
| | | YSFSISNLEPEDNATYYCLQYDNLLTFGAGTKLEL | | LCDR3 | LQYDNLLT | 158 | LCDR3 | LQYDNLLT | 158 |
| | | K | | | | | | | |
| 4540-063 | VH | QVQLVQSGAELKKPGASVKVSCKASGYTFADYYMN | 258 | HCDR1 | GYTFADY | 154 | HCDR1 | DYYMN | 276 |
| | | WVRQAPGQGLEWMGWIFPGSGSTYYNQKFQGRVTM | | HCDR2 | FPGSGS | 155 | HCDR2 | WIFPGSGSTYY | 277 |
| | | TVDKSSSTAYMELSRLRSDDTAVYYCARGDSGRAM | | | | | | NQKFQG | |
| | | DYWGQGTLVTVSS | | HCDR3 | GDSGRAMDY | 156 | HCDR3 | GDSGRAMDY | 156 |
| | VL | DIQMTQSPSSLSASVGDRVTITCQASQDINKYLAW | 261 | LCDR1 | QASQDINKYLA | 274 | LCDR1 | QASQDINKYLA | 274 |
| | | YQHKPGKAPKLLIHYTSTLETGVPSRFSGSGSGTD | | LCDR2 | YTSTLET | 275 | LCDR2 | YTSTLET | 275 |
| | | FTFTISSLQPEDIATYYCLQYDNLLTFGGGTKVEI | | LCDR3 | LQYDNLLT | 158 | LCDR3 | LQYDNLLT | 158 |
| | | K | | | | | | | |
| 4540-033 | VH | QVQLVQSGAEVKKPGASVKVSCKASGYTFADYYIN | 256 | HCDR1 | GYTFADY | 154 | HCDR1 | DYYIN | 159 |
| | | WVRQAPGQGLEWMGWIFPGSGSTYYAQKLQGRVTM | | HCDR2 | FPGSGS | 155 | HCDR2 | WIFPGSGSTYY | 278 |
| | | TTDTSTSTAYMELRSLRSDDTAVYYCARGDSGRAM | | | | | | AQKLQG | |
| | | DYWGQGTLVTVSS | | HCDR3 | GDSGRAMDY | 156 | HCDR3 | GDSGRAMDY | 156 |
| | VL | DIQMTQSPSSLSASVGDRVTITCQASQDINKYLAW | 261 | LCDR1 | QASQDINKYLA | 274 | LCDR1 | QASQDINKYLA | 274 |
| | | YQHKPGKAPKLLIHYTSTLETGVPSRFSGSGSGTD | | LCDR2 | YTSTLET | 275 | LCDR2 | YTSTLET | 275 |
| | | FTFTISSLQPEDIATYYCLQYDNLLTFGGGTKVEI | | LCDR3 | LQYDNLLT | 158 | LCDR3 | LOYDNLLT | 158 |
| | | K | | | | | | | |
| 4237 | VH | QAHLKESGPGLVAPSQSLSITCTVSGFSLTDYDVH | 171 | HCDR1 | GFSLTDY | 163 | HCDR1 | DYDVH | 169 |
| | | WVRQSPGKGLEWLGVIWNDGSTDYNTAFISRLTIS | | HCDR2 | WNDGS | 164 | HCDR2 | VIWNDGSTDYN | 170 |
| | | KDNSKSQVFFKMNSLQADDTAIYYCARNWYGGYWF | | | | | | TAFIS | |
| | | AYWGQGTLVTVSA | | HCDR3 | NWYGGYWFAY | 165 | HCDR3 | NWYGGYWFAY | 165 |
| | VL | DIQMTQSPASLSASAGETVTITCRSSENIYSYLAW | 172 | LCDR1 | RSSENIYSYLA | 166 | LCDR1 | RSSENIYSYLA | 166 |
| | | YQQKQGKSPQLLVYNANALAEGVPSRFSGSGSVTQ | | LCDR2 | NANALAE | 167 | LCDR2 | NANALAE | 167 |
| | | FSLKINSLQPEDFGSYYCQHHYGTPFTFGSGTKLE | | LCDR3 | QHHYGTPFT | 168 | LCDR3 | QHHYGTPFT | 168 |
| | | IK | | | | | | | |
| 4439 | VH | EIQLQQSGAELVKPGASVKISCKASDYSFTGYNMN | 271 | HCDR1 | DYSFTGY | 266 | HCDR1 | GYNMN | 269 |
| | | WVMQSHGKSLEWIGNIHPYYGGTSFNQKFMGKATL | | HCDR2 | HPYYGG | 267 | HCDR2 | NIHPYYGGTSF | 270 |
| | | TADKSSSTAYMQLNSLTSEDSAVYYCARERSNFHA | | | | | | NQKFMG | |
| | | LDYWGQGTSVTVSS | | HCDR3 | ERSNFHALDY | 268 | HCDR3 | ERSNFHALDY | 268 |
| | VL | DIVLTQSPASLTVSLGQRATFSCRASKSVSTSGYS | 272 | LCDR1 | RASKSVSTSGYSYMH | 146 | LCDR1 | RASKSVSTSGY | 146 |
| | | YMHWYQQKPGQPPKLLIYFTSDLEPGVPARFTGSG | | | | | | SYMH | |
| | | SGTDFTLNIHPVEEEDAATYYCQHSRELPYPFGGG | | LCDR2 | FTSDLEP | 147 | LCDR2 | FTSDLEP | 147 |
| | | TKLEIK | | LCDR3 | QHSRELPYP | 148 | LCDR3 | QHSRELPYP | 148 |

TABLE 2
Nucleotide sequences of heavy chain variable regions (VHs) and light chain variable regions (VLs)
of exemplary antibody molecules

| | | | SEQ ID |
|---|---|---|---|
| Antibody | Chain | Nucleotide Sequence | NO |
| 2218 | VH | GATGTACAGCTTCAGGAGTCAGGACCTGGCCTCGTGAAACCTTCTCAGTCTCTGTCTCTCACCTGCTCTGTCACTGGC | 71 |
| | | TACTCCATCACCAGTGGTTATTACTGGAACTGGATCCGGCAGTTTCCAGGAAACAAACTGGAATGGATGGGCTACATA | |
| | | AGCTACGATGGTTACAATAACTACAACCCATCTCTCAAAAATCGAATCTCCATCACTCGTGACACATCTAAGAACCAG | |
| | | TTTTTCCTGAAGTTGAATTCTGTGACTACTGAGGACACAGCCACATATTACTGTGCAAACTACTATGATTACGAAGAC | |
| | | TGGTACTTCGGTGTCTGGGGCACAGGGACCACGGTCACCGTCTCCTCA | |
| | VL | GACATTGTGCTGACCCAATCTCCAGCTTCTTTGGCTATGTCTCTAGGGAAGAGGGCCACCATCTCCTGCAGAGCCAGC | 72 |
| | | GAAAGTGTCAGTATTATTGGTACTAATTCAATACACTGGTACCAACAGAAACCAGGACAGCCACCCAAACTCCTCATC | |
| | | TATCATGCATCCAACCTAGAAACTGGAGTCCCTGCCAGGTTCAGTGGCAGTGGGTCTAGAACAGACTTCACCCTCACC | |
| | | ATTGATCCTGTGGAGGAAGATGATGTTGCAATCTATTACTGTCTGCAAAGTAGGAAGATTCCGTACACGTTCGGAGGG | |
| | | GGGACCAAGCTGGAAATAAAA | |
| 2419 | VH | CAGGTCCAGCTGCAGCAGTCTGGAGCTGAGCTGGTGAAACCCGGGGCATCAGTGAGGCTGTCCTGCGAGGCTTCTGGC | 73 |
| | | TACACCTTCACGGACTATACTATACACTGGGTAAAGCAGAGGTCTGGACAGGGTCTTGAGTGGATTGGATGGATTTAC | |
| | | CCTCTAAGAGGTAGTATAAACTACAATGAGAAATTCAAGGACAAGGCCACATTGACTGCGGACAAATCCTCCAGCACA | |
| | | GTCTATTTGGAGCTTGGTAGATTGACATCTAAGGACTCTGCGGTCTATTTCTGTGCAAGACACGGAGCCTACTATAGT | |
| | | AACGCCTTTGACTACTGGGGCCAAGGCACCACTCTCACAGTCTCCTCA | |
| | VL | AACATTGTAATGACCCAATCTCCAGCTTCATTGGCTGTGTCTCTAGGTCAGAGGGCCACCATCTCCTGCAGAGCCAGC | 74 |
| | | GAGAGTGTTGATAATGATGGCATTAGATTTATGCACTGGTACCAGCAGAAACCAGGACAGCCACCCAAACTCCTCATC | |
| | | TATCGTGCATCCAACCTAGAATCTGGGATCCCTGCCAGGTTCAGTGGCAGTGGGTCTAGGACAGACTTCACCCTCACT | |
| | | ATTAATCCTGTGGAGACTGATGATGTTGCAACCTATTACTGTCAGCAAAGTAATAAGGATCCGTACACGTTCGGAGGG | |
| | | GGGACCAAGCTGGAGCTGAAA | |
| 2419-1305 | VH | CAAGTTCAGTTGGTGCAAAGCGGGGCAGAAGTGAAGAAACCTGGTGCTTCTGTGAAAGTTTCCTGCAAGGCCAGCGGC | 304 |
| | | TACACCTTTACTGATTACACAATACACTGGGTACGGCAGGCAACTGGGCAAGGATTGGAATGGATGGGGTGGATATAC | |
| | | CCATTGCGAGGGTCTATAAACTACGCACAGAAATTTCAAGGTCGAGTAACAATGACAGCCAACAAATCAATAAGCACC | |
| | | GTTTATATGGAACTCTCATCTCTCAGGAGTGAGGATACCGCCGTGTATTTCTGCGCACGACACGGTGCATATTACTCA | |
| | | AACGCTTTCGACTATTGGGGCCAGGGCACCCTTGTGACTGTTAGTAGC | |
| | VL | GAGATAGTAATGACTCAGTCTCCCGCTACACTTAGTGTAAGCCCAGGGGAGCGAGCAACCCTCAGTTGCAGAGCATCT | 305 |
| | | GAGAGTGTTGATAATGATGGAATACGTTTTCTCCATTGGTATCAACAAAAACCAGGGCAGGCCCCCAGATTGCTGATC | |
| | | TACCGTGCTTCCAATCGCGAGACTGGCATTCCTGCACGTTTCAGCGGCAGCGGCTCCGGAACCGAGTTTACACTTACT | |
| | | ATTAGCTCACTCCAGTCTGAAGACTTCGCTGTGTATTACTGTCAGCAATCCAACAAGGACCCATACACTTTCGGAGGC | |
| | | GGCACTAAGGTTGAGATCAAA | |
| 2419-1306 | VH | CAAGTTCAGTTGGTGCAAAGCGGGGCAGAAGTGAAGAAACCTGGTGCTTCTGTGAAAGTTTCCTGCAAGGCCAGCGGC | 304 |
| | | TACACCTTTACTGATTACACAATACACTGGGTACGGCAGGCAACTGGGCAAGGATTGGAATGGATGGGGTGGATATAC | |
| | | CCATTGCGAGGGTCTATAAACTACGCACAGAAATTTCAAGGTCGAGTAACAATGACAGCCAACAAATCAATAAGCACC | |
| | | GTTTATATGGAACTCTCATCTCTCAGGAGTGAGGATACCGCCGTGTATTTCTGCGCACGACACGGTGCATATTACTCA | |
| | | AACGCTTTCGACTATTGGGGCCAGGGCACCCTTGTGACTGTTAGTAGC | |
| | VL | GAGATAGTTATGACTCAGTCTCCCGCCACACTTTCAGTAAGTCCCGGTGAACGCGCCACCCTGTCCTGCCGTGCTTCC | 306 |
| | | GAATCAGTGGATAATGACGGCATTAGGTTTTTGCACTGGTACCAACAAAAGCCCGGACAGGCCCCCCGCCTGCTGATA | |
| | | TATCGTGCATCAACACGAGCAACAGGGATCCCCGCTCGATTTAGTGGATCCGGAAGCAGGACCGAATTTACACTTACC | |
| | | ATTTCCTCACTTCAGTCAGAAGATTTCGCCGTTTACTACTGTCAGCAGTCAAATAAGGATCCTTACACATTTGGGGGC | |
| | | GGTACAAAAGTCGAGATCAAA | |
| 2419-1310 | VH | CAAGTTCAGTTGGTGCAAAGCGGGGCAGAAGTGAAGAAACCTGGTGCTTCTGTGAAAGTTTCCTGCAAGGCCAGCGGC | 304 |
| | | TACACCTTTACTGATTACACAATACACTGGGTACGGCAGGCAACTGGGCAAGGATTGGAATGGATGGGGTGGATATAC | |
| | | CCATTGCGAGGGTCTATAAACTACGCACAGAAATTTCAAGGTCGAGTAACAATGACAGCCAACAAATCAATAAGCACC | |
| | | GTTTATATGGAACTCTCATCTCTCAGGAGTGAGGATACCGCCGTGTATTTCTGCGCACGACACGGTGCATATTACTCA | |
| | | AACGCTTTCGACTATTGGGGCCAGGGCACCCTTGTGACTGTTAGTAGC | |
| | VL | GACATTGTAATGACCCAGTCTCCCGATAGCCTCGCTGTCTCACTCGGAGAACGCGCAACCATCAACTGCAAGTCCTCC | 318 |
| | | CAAAGCGTTGACAATGACGGCATTAGGTTTTTGCACTGGTACCAGCAGAAACCCGGTCAACCTCCTAAGTTGCTCATT | |
| | | TACCGAGCATCTACCCGCGAGTCAGGAGTACCTGATCGCTTTTCCGGTAGCGGTAGTGGAACAGATTTTACTCTGACC | |
| | | ATTAGTTCACTCCAGGCAGAAGATGTGGCTGTCTACTACTGCCAACAGTCAAATAAAGACCCTTATACCTTCGGTGGG | |
| | | GGTACCAAAGTAGAGATCAAA | |
| 2419-0806 | VH | GAGGTCCAGTTGGTCCAGTCAGGAGCCGAAGTCAAGAAGCCTGGGGAAAGCCTGAAAATAAGTTGCAAAGCTAGTGGA | 307 |
| | | TATACATTTACAGATTATACCATTCATTGGGTCCGGCAAATGCCAGGAAAAGGCTTGGAGTGGATGGGGTGGATTTAT | |
| | | CCCCTCCGAGGCTCAATAAATTATAGTCCTAGTTTTCAGGGGCAGGTAACTATTAGCGCTGATAAAAGTATTTCTACA | |
| | | GTTTATTTGCAGTGGAGTTCATTGAAGGCTAGTGACACCGCTATGTATTTCTGCGCTAGACATGGTGCATATTATTCA | |
| | | AATGCCTTCGACTATTGGGGCCAGGGCACCCTCGTCACTGTGAGTTCC | |
| | VL | GAGATAGTTATGACTCAGTCTCCCGCCACACTTTCAGTAAGTCCCGGTGAACGCGCCACCCTGTCCTGCCGTGCTTCC | 306 |
| | | GAATCAGTGGATAATGACGGCATTAGGTTTTTGCACTGGTACCAACAAAAGCCCGGACAGGCCCCCCGCCTGCTGATA | |
| | | TATCGTGCATCAACACGAGCAACAGGGATCCCCGCTCGATTTAGTGGATCCGGAAGCAGGACCGAATTTACACTTACC | |
| | | ATTTCCTCACTTCAGTCAGAAGATTTCGCCGTTTACTACTGTCAGCAGTCAAATAAGGATCCTTACACATTTGGGGGC | |
| | | GGTACAAAAGTCGAGATCAAA | |
| 2419-0205 | VH | CAGGTGCAACTTGTTCAGTCAGGGGCTGAAGTAAAGAAGCCAGGCTCATCAGTCAAGGTATCATGCAAAGCATCTGGC | 308 |
| | | TATACATTTACAGATTACACCATTCACTGGGTGAGGCAAGCTCCCGGTCAAGGTCTCGAGTGGATGGGGTGGATATAC | |
| | | CCTCTCAGAGGCTCTATAAATTACGCTCAGAAATTTCAAGGGAGAGTTACAATTACTGCTGATAAAAGTACCAGCACT | |
| | | GCTTATATGGAGCTTTCCTCACTTCGTTCAGAGGACACCGCCGTTTACTTTTGTGCCCGGCATGGTGCCTATTATTCA | |
| | | AATGCCTTCGATTATTGGGGGCAGGGAACTTTGGTCACAGTTTCATCT | |
| | VL | GAGATAGTAATGACTCAGTCTCCCGCTACACTTAGTGTAAGCCCAGGGGAGCGAGCAACCCTCAGTTGCAGAGCATCT | 305 |
| | | GAGAGTGTTGATAATGATGGAATACGTTTTCTCCATTGGTATCAACAAAAACCAGGGCAGGCCCCCAGATTGCTGATC | |
| | | TACCGTGCTTCCAATCGCGAGACTGGCATTCCTGCACGTTTCAGCGGCAGCGGCTCCGGAACCGAGTTTACACTTACT | |
| | | ATTAGCTCACTCCAGTCTGAAGACTTCGCTGTGTATTACTGTCAGCAATCCAACAAGGACCCATACACTTTCGGAGGC | |
| | | GGCACTAAGGTTGAGATCAAA | |
| 2419-0406 | VH | CAAGTTCAACTTGTCCAAAGTGGGGCTGAAGTTAAAAAACCTGGATCATCAGTCAAGGTTTCATGCAAAGCCAGCGGT | 309 |
| | | TACACATTTACAGACTATACAATACATTGGGTTCGACAGGCTCCCGGGCAAGGGCTCGAATGGATGGGATGGATTTAT | |
| | | CCCCTCAGGGGCTCAATTAACTATGCTGAGAAATTTAAGGGTCGTGTAACACTCACCGCCGATAAATCCACCTCAACC | |
| | | GTATATATGGAGCTTTCTTCTCTTCGCTCTGAAGATACCGCCGTCTATTTCTGCGCACGACACGGGGCATACTATTCT | |
| | | AATGCTTTTGACTACTGGGGACAAGGGACACTTGTGACCGTTAGTAGC | |
| | VL | GAGATAGTTATGACTCAGTCTCCCGCCACACTTTCAGTAAGTCCCGGTGAACGCGCCACCCTGTCCTGCCGTGCTTCC | 306 |
| | | GAATCAGTGGATAATGACGGCATTAGGTTTTTGCACTGGTACCAACAAAAGCCCGGACAGGCCCCCCGCCTGCTGATA | |
| | | TATCGTGCATCAACACGAGCAACAGGGATCCCCGCTCGATTTAGTGGATCCGGAAGCAGGACCGAATTTACACTTACC | |
| | | ATTTCCTCACTTCAGTCAGAAGATTTCGCCGTTTACTACTGTCAGCAGTCAAATAAGGATCCTTACACATTTGGGGGC | |
| | | GGTACAAAAGTCGAGATCAAA | |
| 2419-0605 | VH | CAGGTGCAGTTGGTCCAGAGCGGGGCAGAGGTTAAGAAGCCTGGGGCCTCAGTAAAGGTATCCTGCAAGGCTTCTGGG | 319 |
| | | TACACCTTCACAGATTACACTATTCATTGGGTGCGCCAAGCACCTGGTCAAGGCCTTGAATGGATGGGATGGATTTAC | |
| | | CCCTTGCGAGGGAGTATTAATTATGCACAGAAGTTCCAGGGAAGGGTTACTCTTACCGCCGACAAGTCCACATCAACC | |
| | | GTTTACATGGAGCTTTCCTCTCTCAGGTCCGAAGACACTGCTGTATATTTCTGCGCTCGGCATGGGGCTTATTACAGC | |
| | | AACGCCTTCGATTACTGGGGTCAGGGTACATTGGTCACAGTGTCCAGT | |
| | VL | GAGATAGTAATGACTCAGTCTCCCGCTACACTTAGTGTAAGCCCAGGGGAGCGAGCAACCCTCAGTTGCAGAGCATCT | 305 |
| | | GAGAGTGTTGATAATGATGGAATACGTTTTCTCCATTGGTATCAACAAAAACCAGGGCAGGCCCCCAGATTGCTGATC | |
| | | TACCGTGCTTCCAATCGCGAGACTGGCATTCCTGCACGTTTCAGCGGCAGCGGCTCCGGAACCGAGTTTACACTTACT | |
| | | ATTAGCTCACTCCAGTCTGAAGACTTCGCTGTGTATTACTGTCAGCAATCCAACAAGGACCCATACACTTTCGGAGGC | |
| | | GGCACTAAGGTTGAGATCAAA | |
| 2419-0805 | VH | GAGGTCCAGTTGGTCCAGTCAGGAGCCGAAGTCAAGAAGCCTGGGGAAAGCCTGAAAATAAGTTGCAAAGCTAGTGGA | 307 |
| | | TATACATTTACAGATTATACCATTCATTGGGTCCGGCAAATGCCAGGAAAAGGCTTGGAGTGGATGGGGTGGATTTAT | |
| | | CCCCTCCGAGGCTCAATAAATTATAGTCCTAGTTTTCAGGGGCAGGTAACTATTAGCGCTGATAAAAGTATTTCTACA | |
| | | GTTTATTTGCAGTGGAGTTCATTGAAGGCTAGTGACACCGCTATGTATTTCTGCGCTAGACATGGTGCATATTATTCA | |
| | | AATGCCTTCGACTATTGGGGCCAGGGCACCCTCGTCACTGTGAGTTCC | |
| | VL | GAGATAGTAATGACTCAGTCTCCCGCTACACTTAGTGTAAGCCCAGGGGAGCGAGCAACCCTCAGTTGCAGAGCATCT | 305 |
| | | GAGAGTGTTGATAATGATGGAATACGTTTTCTCCATTGGTATCAACAAAAACCAGGGCAGGCCCCCAGATTGCTGATC | |
| | | TACCGTGCTTCCAATCGCGAGACTGGCATTCCTGCACGTTTCAGCGGCAGCGGCTCCGGAACCGAGTTTACACTTACT | |
| | | ATTAGCTCACTCCAGTCTGAAGACTTCGCTGTGTATTACTGTCAGCAATCCAACAAGGACCCATACACTTTCGGAGGC | |
| | | GGCACTAAGGTTGAGATCAAA | |
| 2419-0105 | VH | CAAGTGCAGTTGGTCCAGAGTGGAGCAGAGGTGAAGAAGCCTGGTGCTTCCGTCAAGGTGAGTTGCAAGGCATCTGGT | 310 |
| | | TATACTTTCACTGACTACACAATTCATTGGGTCAGGCAGGCCCCTGGACAGGGACTGGAATGGATGGGATGGATCTAT | |
| | | CCACTTAGAGGATCAATCAACTATGCTCAAAAGTTCCAGGGTCGTGTAACAATGACCGCAGACAAAAGTATCTCAACT | |
| | | GTATACATGGAATTGTCCCGATTGAGGAGCGACGACACAGCCGTATATTATTGTGCCAGGCACGGAGCCTACTACAGT | |
| | | AATGCCTTCGACTACTGGGGGCAGGGCACCCTTGTTACCGTGTCCAGC | |
| | VL | GAGATAGTAATGACTCAGTCTCCCGCTACACTTAGTGTAAGCCCAGGGGAGCGAGCAACCCTCAGTTGCAGAGCATCT | 305 |
| | | GAGAGTGTTGATAATGATGGAATACGTTTTCTCCATTGGTATCAACAAAAACCAGGGCAGGCCCCCAGATTGCTGATC | |
| | | TACCGTGCTTCCAATCGCGAGACTGGCATTCCTGCACGTTTCAGCGGCAGCGGCTCCGGAACCGAGTTTACACTTACT | |
| | | ATTAGCTCACTCCAGTCTGAAGACTTCGCTGTGTATTACTGTCAGCAATCCAACAAGGACCCATACACTTTCGGAGGC | |
| | | GGCACTAAGGTTGAGATCAAA | |
| 2419-1204 | VH | CAAGTGCAGCTCGTTCAGTCTGGCGCAGAAGTGAAGAAGCCAGGAGCTTCCGTTAAAGTGTCCTGTAAAGCCTCTGGA | 311 |
| | | TATACATTCACAGATTATACAATTCACTGGGTGAGACAAGCAACCGGTCAAGGTCTCGAATGGATGGGCTGGATATAC | |
| | | CCCCTCCGAGGTTCCATCAACTACGCTCAAAAATTCCAAGGACGAGTCACTATGACAGCAAACAAGAGTTCCTCCACT | |
| | | GTATATATGGAACTCTCTAGTTTGCGCTCTGAAGACACCGCCGTGTACTTCTGTGCCAGGCACGGCGCATACTATTCT | |
| | | AATGCATTTGACTATTGGGGGCAGGGCACATTGGTAACAGTTAGTTCC | |
| | VL | GAAATTGTAATGACCCAGAGCCCCGCCACCCTTAGTGTGTCCCCAGGCGAGAGGGCCACTCTTTCTTGCCGCGCAAGC | 312 |
| | | GAATCCGTAGACAACGATGGTATAAGATTTTTGCATTGGTATCAGCAAAAGCCAGGCCAGGCACCCCGGCTTCTCATC | |
| | | TACAGAGCTAGCACCCTCGAAACTGGAATCCCCGCTCGTTTTTCAGGATCTGGTAGCGGAACAGAATTTACTTTGACA | |
| | | ATTAGTAGTTTGCAGTCAGAGGACTTTGCTGTCTATTATTGCCAGCAGTCTAATAAAGATCCATACACCTTCGGCGGA | |
| | | GGGACCAAAGTAGAGATTAAA | |
| 2419-1210 | VH | CAAGTGCAGCTCGTTCAGTCTGGCGCAGAAGTGAAGAAGCCAGGAGCTTCCGTTAAAGTGTCCTGTAAAGCCTCTGGA | 311 |
| | | TATACATTCACAGATTATACAATTCACTGGGTGAGACAAGCAACCGGTCAAGGTCTCGAATGGATGGGCTGGATATAC | |
| | | CCCCTCCGAGGTTCCATCAACTACGCTCAAAAATTCCAAGGACGAGTCACTATGACAGCAAACAAGAGTTCCTCCACT | |
| | | GTATATATGGAACTCTCTAGTTTGCGCTCTGAAGACACCGCCGTGTACTTCTGTGCCAGGCACGGCGCATACTATTCT | |
| | | AATGCATTTGACTATTGGGGGCAGGGCACATTGGTAACAGTTAGTTCC | |
| | VL | GACATTGTAATGACCCAGTCTCCCGATAGCCTCGCTGTCTCACTCGGAGAACGCGCAACCATCAACTGCAAGTCCTCC | 318 |
| | | CAAAGCGTTGACAATGACGGCATTAGGTTTTTGCACTGGTACCAGCAGAAACCCGGTCAACCTCCTAAGTTGCTCATT | |
| | | TACCGAGCATCTACCCGCGAGTCAGGAGTACCTGATCGCTTTTCCGGTAGCGGTAGTGGAACAGATTTTACTCTGACC | |
| | | ATTAGTTCACTCCAGGCAGAAGATGTGGCTGTCTACTACTGCCAACAGTCAAATAAAGACCCTTATACCTTCGGTGGG | |
| | | GGTACCAAAGTAGAGATCAAA | |
| 2419-1406 | VH | CAAGTTCAGTTGGTGCAAAGCGGGGCAGAAGTGAAGAAACCTGGTGCTTCTGTGAAAGTTTCCTGCAAGGCCAGCGGC | 313 |
| | | TACACCTTTACTGATTACACAATACACTGGGTACGGCAGGCAACTGGGCAAGGATTGGAATGGATGGGGTGGATATAC | |
| | | CCATTGCGAGGGTCTATAAACTACGCACAGAAATTTCAAGGTCGAGTAACAATGACAGCCGACAAATCAATAAGCACC | |
| | | GTTTATATGGAACTCTCATCTCTCAGGAGTGAGGATACCGCCGTGTATTTCTGCGCACGACACGGTGCATATTACTCA | |
| | | AACGCTTTCGACTATTGGGGCCAGGGCACCCTTGTGACTGTTAGTAGC | |
| | VL | GAGATAGTTATGACTCAGTCTCCCGCCACACTTTCAGTAAGTCCCGGTGAACGCGCCACCCTGTCCTGCCGTGCTTCC | 306 |
| | | GAATCAGTGGATAATGACGGCATTAGGTTTTTGCACTGGTACCAACAAAAGCCCGGACAGGCCCCCCGCCTGCTGATA | |
| | | TATCGTGCATCAACACGAGCAACAGGGATCCCCGCTCGATTTAGTGGATCCGGAAGCAGGACCGAATTTACACTTACC | |
| | | ATTTCCTCACTTCAGTCAGAAGATTTCGCCGTTTACTACTGTCAGCAGTCAAATAAGGATCCTTACACATTTGGGGGC | |
| | | GGTACAAAAGTCGAGATCAAA | |
| 2419-1205 | VH | CAAGTGCAGCTCGTTCAGTCTGGCGCAGAAGTGAAGAAGCCAGGAGCTTCCGTTAAAGTGTCCTGTAAAGCCTCTGGA | 311 |
| | | TATACATTCACAGATTATACAATTCACTGGGTGAGACAAGCAACCGGTCAAGGTCTCGAATGGATGGGCTGGATATAC | |
| | | CCCCTCCGAGGTTCCATCAACTACGCTCAAAAATTCCAAGGACGAGTCACTATGACAGCAAACAAGAGTTCCTCCACT | |
| | | GTATATATGGAACTCTCTAGTTTGCGCTCTGAAGACACCGCCGTGTACTTCTGTGCCAGGCACGGCGCATACTATTCT | |
| | | AATGCATTTGACTATTGGGGGCAGGGCACATTGGTAACAGTTAGTTCC | |
| | VL | GAGATAGTAATGACTCAGTCTCCCGCTACACTTAGTGTAAGCCCAGGGGAGCGAGCAACCCTCAGTTGCAGAGCATCT | 305 |
| | | GAGAGTGTTGATAATGATGGAATACGTTTTCTCCATTGGTATCAACAAAAACCAGGGCAGGCCCCCAGATTGCTGATC | |
| | | TACCGTGCTTCCAATCGCGAGACTGGCATTCCTGCACGTTTCAGCGGCAGCGGCTCCGGAACCGAGTTTACACTTACT | |
| | | ATTAGCTCACTCCAGTCTGAAGACTTCGCTGTGTATTACTGTCAGCAATCCAACAAGGACCCATACACTTTCGGAGGC | |
| | | GGCACTAAGGTTGAGATCAAA | |
| 2419-0206 | VH | CAGGTGCAACTTGTTCAGTCAGGGGCTGAAGTAAAGAAGCCAGGCTCATCAGTCAAGGTATCATGCAAAGCATCTGGC | 308 |
| | | TATACATTTACAGATTACACCATTCACTGGGTGAGGCAAGCTCCCGGTCAAGGTCTCGAGTGGATGGGGTGGATATAC | |
| | | CCTCTCAGAGGCTCTATAAATTACGCTCAGAAATTTCAAGGGAGAGTTACAATTACTGCTGATAAAAGTACCAGCACT | |
| | | GCTTATATGGAGCTTTCCTCACTTCGTTCAGAGGACACCGCCGTTTACTTTTGTGCCCGGCATGGTGCCTATTATTCA | |
| | | AATGCCTTCGATTATTGGGGGCAGGGAACTTTGGTCACAGTTTCATCT | |
| | VL | GAGATAGTTATGACTCAGTCTCCCGCCACACTTTCAGTAAGTCCCGGTGAACGCGCCACCCTGTCCTGCCGTGCTTCC | 306 |
| | | GAATCAGTGGATAATGACGGCATTAGGTTTTTGCACTGGTACCAACAAAAGCCCGGACAGGCCCCCCGCCTGCTGATA | |
| | | TATCGTGCATCAACACGAGCAACAGGGATCCCCGCTCGATTTAGTGGATCCGGAAGCAGGACCGAATTTACACTTACC | |
| | | ATTTCCTCACTTCAGTCAGAAGATTTCGCCGTTTACTACTGTCAGCAGTCAAATAAGGATCCTTACACATTTGGGGGC | |
| | | GGTACAAAAGTCGAGATCAAA | |
| 2621 | VH | GAGGTCCAGCTTCAGCAGTCTGGAGCTGAGCTGGTGAGGCCTGGGTCCTCAGTGAAGATGTCCTGCAAGACTTCTGGA | 75 |
| | | TATACTTTCACAAGCTACGGTATAAACTGGGTGAAGCAGAGGCCTGGACAGGGCCTGGAATGGATTGGATATATTTAT | |
| | | ATTGGAAATGGTTATGCTGAGTACAATGAGAGGTTCAAGGGCAAGGCCACACTGACTTCAGACACATCCTCCAGCACA | |
| | | GCCTACATGCAGCTCAGCAGCCTGACATCTGAGGACTCTGCAATCTATTTCTGTGCACTATACTATCCCTGGTTTACT | |
| | | TACTGGGGCCAGGGGACTCTGGTCACTGTCTCTGCA | |
| | VL | GACATCCAGATGACTCAGTCTCCAGCCTCCCTTTCTGCATCTGTGGGAGATTCTGTCACCATCACATGTCGAGCAAGT | 76 |
| | | GAGAATATTTACAGTTATTTAGCATGGTATCAGCAGAAACAGGGAAAATCTCCTCAGCTCCTGGTCTATAATGCAAAA | |
| | | ACCTTAGCTGAAGGTGTGCCATCAAGGTTCAGTGGCAGTGGATCAGGCACACAGTTTTCTCTGAAGATCAACAGCCTG | |
| | | CAGCCTGAAGATTTTGGGAATTATTACTGTCAACATCATTATGATACTCCGTTCACGTTCGGAGGGGGGACCAAGCTG | |
| | | GAAATAAAA | |
| 2922 | VH | CAGGTTCAGCTGCACCAGTCTGGACCTGAGCTGGTGAAGCCTGGGGCTTCAGTGAAGTTGTCCTGCAAGACTTCTGGC | 77 |
| | | TACACCTTCACAAGCTACGATGTCTTCTGGGTGAAGCAGAGGCCTGGACAGGGACTTGAGTGGATTGGATGGATTTAT | |
| | | CCTAGAGATAGTAGTACTAAATACAATGAGAAGTTCAAGGGCAAGGCCACATTGACTGTAGACACATCCTCCAGCACA | |
| | | GCATACATGGAGCTCCACAGCCTGACATCTGAGGACTCTGCGGTCTATTTCTGTGCAAAAGAGGGGTATGATTATGAC | |
| | | AAGAGGGGCTTTGACTACTGGGGCCAAGGCACCACTCTCACAGTCTCCTCA | |
| | VL | GACATTGTGCTGACCCAATCTCCAGCTTCTTTGGCTGTGTCTCTAGGGCAGAGGGCCATCATCTCCTGCAAGGCCAGC | 78 |
| | | CAAAGTGTCAGTTTTGCTGGTACTAATTTAATGCACTGGTACCAACAGAGACCAGGGCAGCAACCCAAACTCCTCATC | |
| | | TATCGTGCATCCAACCTAGAACCTGGGGTTCCTACCAGGTTTAGTGGCAGTGGGTCTAGGACAGACTTCACCCTCAAT | |
| | | ATCCATCCTGTGGAGGAAGATGATGCTGCAACCTATTACTGTCAGCAAAGTAGGGAATATCCGTGGACGTTCGGTGGA | |
| | | GGCACCAAGCTGGAAATCAAA | |
| 3125 | VH | CAGGTTCAACTGCAGCAGTCTGGGGCTGAGCTGGTGAGGCCTGGGGCTTCAGTGACGCTGTCCTGCAAGGCTTCGGGC | 79 |
| | | TACACTTTTACTGACTATGAAATGCACTGGGTGAAGCAGACACCTGTGCATGGCCTGGAATGGATTGGAGCTATTGAT | |
| | | CCTGAAACTGGTGGTACTGCCTACAATCAGAGGTTCAAGGGCAAGGCCATACTGACTACAGACAAATCCTCCATCACA | |
| | | GCCTACATGGAGCTCCGCAGCCTGACATCTGAGGACTCTGCCGTCTATTACTGTACAAGATGGAATGATGGCGACTAC | |
| | | TGGGGCCAAGGCACCACTCTCACAGTCTCCTCA | |
| | VL | GATGTTGTGATGACCCAGACTCCACTGTCTTTGTCGGTTACCATTGGACAACCAGCCTCCATTTCTTGCAAGTCAAGT | 80 |
| | | CAGAGCCTCTTATACAGTAATGGAAAGACATATTTGAATTGGTTTCAACAGAGGCCTGGCCAGTCTCCAAAGCGCCTA | |
| | | ATGTATCAGGTGTCCAAACTGGACCCTGGCATCCCTGACAGGTTCAGTGGCAGTGGATCAGAAACAGATTTTACACTT | |
| | | AAAATCAGCAGAGTGGAGGCTGAAGATTTGGGACTTTATTACTGCTTGCAAGGTACATATTATCCGTACACGTTCGGA | |
| | | GGGGGGACCAAGCTGGAAATAAAA | |
| 3327 | VH | GAGGTCCAGCTGCAGCAGTCTGGACCTGAGCTGGTGAAGCCTGGGGCTTCAGTGAAGATGTCCTGCAAGGCTTCTGGT | 81 |
| | | TACTCCTTTACTGGCTACTTTATGAACTGGGTGAAGCAGAGCCATGGAAAGAGCCTTGAGTGGATTGGACGTATTAAT | |
| | | CCTTACAATGGTGATACTTTCTACAACCAGAAGTTCAAGGGCAAGGCCACATTGACTGTAGACAAATCCTCTAGCACA | |
| | | GCCCACATGGAGCTCCGGAGCCTGACATCTGAGGACTCTGCACTCTATTATTGTGCAAGCGAAGGTGATGGTTACTAC | |
| | | TGGTACTTCGATGTCTGGGGCGCAGGGACCACGGTCACCGTCTCCTCA | |
| | VL | GACATTGTGCTGACCCAATCTCCAGCTTCTTTGGCTGTGTCTCTAGGGCAGAGGGCCACCATCTCCTGCAGAGCCAGC | 82 |
| | | GAAAGTGTTGATAATTATGGCATTAGTTTTATGAACTGGTTCCAACAGAAACCAGGACAGCCACCCAAACTCCTCATC | |
| | | TATGCTGCATCCAACCAAGGATCCGGGGTCCCTGCCAGGTTTAGTGGCAGTGGGTCTGGGACAGACTTCAGCCTCAAC | |
| | | ATCCATCCTATGGAGGAGGATGATACTGCAATGTATTTCTGTCAGCAAAGTAAGGAGGTTCCTCGGACGTTCGGTGGA | |
| | | GGCACCAAGCTGGAAATCAAA | |
| 3525 | VH | CAGGTTCAACTGCAGCAGTCTGGGGCTGAGCTGGTGAGGCCTGGGGCTTCAGTGAAGCTGTCCTGCAAGGCTTCGGGC | 83 |
| | | TACACATTTACTGACCATGAAATGCACTGGGTGAGACAGACACCTGTGCATGGCCTGGAATGGATTGGAGTTATTGAT | |
| | | CCTGACACTGGTGATACTACCTACAATCAGAAATTCAAGGGCAAGGCCACACTGACTGCAGACAAATCCTCCAGCACA | |
| | | GCCTACATGGACCTCCGCAGCCTGACATCTGAGGACTCTGCCGTCTTTTACTGTACACGGTGGACTGGGGGGGACTAC | |
| | | TGGGGCCATGGCACCACTCTCACAGTCTCCTCA | |
| | VL | GATGTTGTGATGACCCAGACTCCACTGTCTTTGTCGGTTACCATTGGACAACCAGCCTCCATTTCTTGCAAGTCAAGT | 80 |
| | | CAGAGCCTCTTATACAGTAATGGAAAGACATATTTGAATTGGTTTCAACAGAGGCCTGGCCAGTCTCCAAAGCGCCTA | |
| | | ATGTATCAGGTGTCCAAACTGGACCCTGGCATCCCTGACAGGTTCAGTGGCAGTGGATCAGAAACAGATTTTACACTT | |
| | | AAAATCAGCAGAGTGGAGGCTGAAGATTTGGGACTTTATTACTGCTTGCAAGGTACATATTATCCGTACACGTTCGGA | |
| | | GGGGGGACCAAGCTGGAAATAAAA | |
| 3530 | VH | CAGGTTCAACTGCAGCAGTCTGGGGCTGAGCTGGTGAGGCCTGGGGCTTCAGTGAAGCTGTCCTGCAAGGCTTCGGGC | 83 |
| | | TACACATTTACTGACCATGAAATGCACTGGGTGAGACAGACACCTGTGCATGGCCTGGAATGGATTGGAGTTATTGAT | |
| | | CCTGACACTGGTGATACTACCTACAATCAGAAATTCAAGGGCAAGGCCACACTGACTGCAGACAAATCCTCCAGCACA | |
| | | GCCTACATGGACCTCCGCAGCCTGACATCTGAGGACTCTGCCGTCTTTTACTGTACACGGTGGACTGGGGGGGACTAC | |
| | | TGGGGCCATGGCACCACTCTCACAGTCTCCTCA | |
| | VL | GATGCTGTGATGACCCAGACTCCACTGTCTTTGTCGGTTACCATTGGACAACCAGCCTCTATCTCTTGCAAGTCGAGT | 84 |
| | | CAGAGCCTCTTATATAGTGATGGAAAGACATATTTGAATTGGTTCCAACAGAGGCCAGGCCAGTCTCCAAAGCGCCTA | |
| | | ATGTATCAGGTGTCCAAACTGGACCCTGGCATCCCTGACAGGTTCAGTGGCAGTGGATCAGAGACAGATTTTACACTT | |
| | | AAAATCAGCAGAGTGGAGGCTGAGGATTTGGGAGTTTATTACTGCTTGCAAGGTACATATTATCCGTATACGTTCGGA | |
| | | TCGGGGACCAAGCTGGAAATAAAA | |
| 4035 | VH | CAGGTGCAGCTGAAGGAGTCAGGACCTGGCCTGGTGGCGCCCTCACAGAGCCTGTCCATCACCTGCACAGTCTCTGGT | 173 |
| | | TTCTCATTAACCATCTATGATGTACACTGGGTTCGCCAGTCTCCAGGAAAGGGTCTGGAGTGGCTGGGAGTGATATGG | |
| | | AGTGATGGAAGCACAGACTATAATGCAGCTTTCATATCTAGACTGAGCATCAGCAAGGACAACTCCAAGAGCCAAGTT | |
| | | TTCTTTAAAATGAACAGTCTGCAAGCTGATGACACAGCCATATACTACTGTGCCAGAAATTGGGTCGACCAGGCCTGG | |
| | | TTTGCTTACTGGGGCCAAGGGACTCTGGTCACTGTCTCTGCA | |
| | VL | GACATCCAGATGACTCAGTCTCCAGCCTCCCTATCTGCATCTGTGGGAGAAACTATCACCATCACATGTCGAGCAAGT | 174 |
| | | AAGAATATTTACAGTTATTTAGCATGGTATCAGCAGAAACAGGGAAAATCTCCTCAGCTCCTGGTCTATAATGCAAAA | |
| | | ACCTTACCAGAAGGTGTGCCATCAAGGTTCAGTGGCAGTGGATCAGGCACACAGTTTTCTCTGAAGATCAACAGCCTG | |
| | | CAGCCTGAAGATTTTGGGAGTTATTACTGTCAACATCATTATGGTACTCCGCTCACGTTCGGTGCTGGGACCAAGCTG | |
| | | GAGCTGAAA | |
| 4035-062 | VH | CAGGTACAACTCCAGGAATCCGGGCCTGGGCTCGTCAAACCAAGCGAAACACTCTCTCTCACCTGCACCGTTTCTGGG | 299 |
| | | TTTTCTCTTACTATCTATGACGTACATTGGGTAAGGCAACCACCCGGGAAGGGGCTGGAGTGGATCGGTGTAATCTGG | |
| | | TCAGATGGATCTACAGACTACAACCCATCCCTTAAAAGCAGGGTGACCATTTCTAAGGACACTTCCAAGAACCAAGTA | |
| | | TCCCTTAAATTGTCCTCTGTAACCGCAGCAGACACCGCAGTTTACTACTGCGCACGAAATTGGGTTGACCAAGCATGG | |
| | | TTTGCATATTGGGGACAGGGAACTCTTGTCACTGTGTCTTCA | |
| | VL | GATATTCAAATGACCCAATCCCCCTCATCACTTTCAGCATCTGTCGGTGATCGGGTCACCATTACTTGCAGAGCCAGT | 300 |
| | | AAGAATATCTACAGCTACCTGGCTTGGTATCAGCAAAAACCTGGTAAGGCCCCTAAACTTCTCGTTTACAATGCTAAG | |
| | | ACCCTTCCCGAGGGAGTTCCTTCCAGGTTTTCCGGTAGCGGGAGTGGAACAGATTTCACCTTGACTATTTCTAGCTTG | |
| | | CAGCCCGAGGATTTCGCTACATACTACTGCCAGCATCACTATGGAACCCCCCTGACCTTCGGTCAGGGAACCAAGCTC | |
| | | GAGATCAAA | |
| 3934 | VH | CAGGTCCAACTGCAGCAGTCTGGACCTGAGCTGGTGAAGCCTGGAGCTTCAGTGAAGCTGTCCTGCAAGGCTGCTGGC | 175 |
| | | TACATCTTCACTGACTATACTATAAACTGGGTGAAGCAGAGTCCTGGACAGGGACTTGAGTGGATTGGATGGATTTAT | |
| | | CCTGGAAGTGGTAATCGTAAATACAATGACAAGTTCAAGGGCAAGGCCACAATGACTGCAGACAAATCCTCCAGCACA | |
| | | GCCTACATGCAGCTCAGCAGCCTGACCTCTGAGGATTCTGCGGTCTATTTCTGTGCAAGAGAGAGTAACTACGTGGGG | |
| | | TACTATGCTATGGACTATTGGGGTCAAGGAACCTCAGTCACCGTCTCCTCA | |
| | VL | GATGTTTTGATGACCCAAACTCCACTCTCCCTGCCTGTCAGTCTTGGAGATCAAGCCTCCATCTCTTGCAGATCTAGT | 176 |
| | | CAGAGCGTTGTAAATAGTAATGGAAACACCTATTTAGAATGGTACCTGCAGAAACCAGGCCAGTCTCCAAATCTCCTG | |
| | | ATCTACAAAGTTTCCAATCGATTTTCTGGGGTCCCAGACAGGTTCAGTGGCAGTGGATCGGGGACAGATTTCACACTC | |
| | | AAGATCAGCAGAGTGGAGGCTGAGGATCTGGGAGTTTATTACTGTTTTCAAGGTTCACATGTTCCGTGGACGTTCGGT | |
| | | GGAGGCACCAAGCTGGAAATCAAA | |
| 3833 | VH | CAGGTCCAGCTGCAGCAGTCTGGAGCTGAGCTGGTAAGGCCTGGGACTTCAGTGAAGATGTCCTGCAAGGCTGCTGGA | 177 |
| | | TACACCTTCACAAACTACTGGATAGGTTGGGTAAAGCAGAGGCCTGGACATGGCCTTGAGTGGATTGGAGATATTTAC | |
| | | CCTGGAGGTATAGGAGGTGGTTATACTAAGTACAATGAGAAGTTCAAGGGCAAGGCCACACTGACTGCAGACACATCC | |
| | | TCCAGCACAGCCTACATGCAGCTCGGCAGCCTGACATCTGAGGACTCTGCCATCTATTTCTGTTCAAGATCGGAAACT | |
| | | GGACGGGCTATGGACTACTGGGGTCAAGGAACCTCAGTCACCGTCTCCTCA | |
| | VL | GACATCCAGATGACACAGTCTCCATCCTCACTGTCTGCATCTCTGGGAGGCAAAGTCACCATCACTTGCAAGGCAAGC | 178 |
| | | CAAGACATTAATAAGTATATAGCTTGGTACCAACACAAGCCTGGAAAAGGTCCTAGGCTGCTCATACATTACACATCT | |
| | | ACATTAAAGCCAGGCATCCCATCAAGGTTCAGTGGAAGTGGGTCTGGGAGAGATTATTCCTTCAGCATCAGTGACCTG | |
| | | GAGCCTGAAGATATTGCAACTTATTATTGTCTACAGTATGATAATCTGAACACGTTCGGAGGGGGGACCAAGCTGGAA | |
| | | ATAAAA | |
| 3631 | VH | GAGATCCAGCTGCAGCAGTCTGGACCTGAGCTGGTGAAGCCTGGGGCTTCAGTGAAGGTGTCCTGCAAGGCTTCTGGT | 179 |
| | | TATTCATTCACTGACTACAACATCTACTGGGTGAAGCAGAGCCATGGAAAGAGCCTTGAGTGGATTGGATATATTGAT | |
| | | CCTTCCAATGGTGGTCCTGGCTACAACCAGAAGTTCAGGGGCAAGGCCACATTGACTGTTGACAAGTCCTCCAGCACA | |
| | | GCCTTCCTGCATCTCAACAGCCTGACATCTGAGGACTCTGCAGTCTATTACTGTGCAAGAAGGGACAACTACGGCTCG | |
| | | GGGACTATGGACTACTGGGGTCAAGGAACCTCAGTCACCGTCTCCTCA | |
| | VL | GACATTGTGATGACCCAGTCTCAAAAATTCATGTCCACATCAGTAGGAGACAGGGTCAGCATCACCTGTAAGGCCAGT | 180 |
| | | CAGAATGTGGGTACTGATGTATCCTGGTATCAACAGAAACCAGGGAAATCTCCTAAACCACTGATTTACTGGGCATCA | |
| | | AACCGGTTCACTGGAGTCCCTGATCGCTTCATAGGTAGTGGATCTGGGACAGATTTCACTCTCACCATCAGCAATGTG | |
| | | CAGTCTGAAGACTTGGCAGATTATTTCTGTGAGCAATATAGCATCTATCCGCTCACGTTCGGTGCTGGGACCAAGCTG | |
| | | GAGCTGAAA | |
| 3732 | VH | GAGATCCAGCTGCAGCAGTCTGGACCTGAGCTGGTGAAGCCTGGGGCGTCAGTGAAGGTATCCTGCAAGGCTTCTGGT | 181 |
| | | TACTCATTCACTGACGACAACATGTACTGGGTGAAGCAGAGCCATGGAAAGAGCCTTGAGTGGATTGGATATATTGAT | |
| | | CCTCTCAATGGTGGTACTGGCTACAACCAGAAATTCAAGGGCAAGGCCACACTGACTGTTGACAAGTCCTCCAGCACA | |
| | | GCCTTCCTGCATCTCAACAGCCTGACATCTGAGGACTCTGCAGTCTATTACTGTGCAAGAAGGGACAACTACGCCACG | |
| | | GGGACTATGGACTACTGGGGTCAAGGAACCTCAGTCACCGTCTCCTCA | |
| | VL | GACATTGTGATGACCCAGTCTCAAAAATTCATGTCCACATCAGTAGGAGACAGGGTCAGCATCACCTGCAAGGCCAGT | 182 |
| | | AAGAATGTGGGTACTGATGTATCCTGGTATCAACAGAAACCAGGGAAATCTCCTAAACCACTGATTTACTGGGCATCA | |
| | | AACCGGTTCACTGGAGTCCCTGATCGCTTCACAGGCAGTGGATCTGGGACAGATTTCACTCTCACCATCAACAATGTG | |
| | | CAGTCTGAAGACTTGGCAGATTATTTCTGTGAGCAATATAGCAGCTATCCGCTCACGTTCGGTGCTGGGACCAAGCTG | |
| | | GAGCTGAAA | |
| 4338 | VH | GAGGTCCAGCTGCAGCAGTCTGGCCCTGAGCTGGTGAAGCCTGGGGCTTCAGTGAAGATATCCTGCAAGGCTTCTGGA | 183 |
| | | TACACATTCACTGACTACAACATGGACTGGGTGAAGCAGAGCCATGGAAAGAGCCTTGAGTGGATTGGAAATATTTAT | |
| | | CCTATCAATGGTTATACTGGCTACAACCAGAGGTTCAAGAACAAGGCCACATTGACTGTAGACAAGTCCTCCAGCACA | |
| | | GCCTACATGGAACTCCACAGCCTGACATCTGAGGACTCTGCGGTCTATTACTGCGCAAGAGATAGTAACTACGTTGGC | |
| | | TGGTACTTCGATGTCTGGGGCGCAGGGACCACGGTCACCGTCTCCTCA | |
| | VL | GATGTTGTGATGACCCAAACTCCACTCTCCCTGCCTGTCAGTCTTGGAGATCAAGCCTCCATCTCTTGCAGATCTAGT | 184 |
| | | CAGAGCCTTGTACACAGTAATGGAAACACCTATTTACATTGGTACCTGCAGAAGCCAGGCCAGTCTCCAAAGCTCCTG | |
| | | ATCTACAAAGTTTCCAACCGATTTTCTGGGGTCCCAGACAGGTTCAGTGGCAGTGGATCAGGGACAGATTTCACATTC | |
| | | AAGATCAGCAGAGTGGAGGCTGAGGATCTGGGAGTTTATTTCTGCTCTCAAAGTACACATGTTCCTCGGACGTTCGGT | |
| | | GGAGGCACCAAGCTGGAAATCAAA | |
| | VL | GACATTGTGCTGACACAGTCTCCTGCTTCCTTAACTGTATCTCTGGGGCAGAGGGCCACCTTCTCATGCAGGGCCAGC | 185 |
| | | AAAAGTGTCAGTACATCTGGCTATAGTTATATGCACTGGTACCAACAGAAACCAGGACAGCCACCCAAACTCCTCATC | |
| | | TATTTTACATCCGACCTAGAACCTGGGGTCCCTGCCAGGTTCACTGGCAGTGGGTCTGGGACAGACTTCACCCTCAAC | |
| | | ATCCATCCTGTGGAGGAGGAGGATGCTGCAACCTATTACTGTCAGCACAGTAGGGAGCTTCCGTACCCCTTCGGAGGG | |
| | | GGGACCAAGTTGGAAATAAAA | |
| 4540 | VH | caggtccagctacagcagtctggacctgagctggtgaagcctggggcttcagtgaagatatcctgcaaggcttctggc | 186 |
| | | tacaccttcgctgactactatataaactgggtgaagcagaggcctggacagggacttgagtggattggatggattttt | |
| | | cctggaagtggtagtacttactacaatgagaagttcaagggcaaggccacacttactgtagacaaatcctccagcaca | |
| | | gcctacatgttgctcagcagcctgacctctgaggactctgcggtctatttctgtgcaagaggggactccggtagggct | |
| | | atggactactggggtcaaggaacctcagtcaccgtctcctca | |
| | VL | gacatccagatgacacagtctccatcctcactgtctgcatctctgggaggcaaagtcaccatcacttgcaaggcaagc | 187 |
| | | caagacattaacaaatatatagcttggtaccaacacaagcctggaaaaggtcctaggctgctcatacattacacatct | |
| | | acattacagtcaggcatcccatcaaggttcagtggaagtgggtctgggagagattattccttcagcatcagcaacctg | |
| | | gagcctgaagataatgcaacttattattgtctacagtatgataatcttctcacgttcggtgctgggaccaagctggag | |
| | | ctgaaa | |
| 4540-063 | VH | CAAGTCCAGCTCGTACAGAGCGGGGCAGAGCTGAAGAAGCCTGGGGCCTCCGTCAAGGTCTCCTGTAAGGCTTCTGGT | 301 |
| | | TACACATTTGCCGACTACTACATGAACTGGGTACGGCAAGCCCCAGGTCAAGGGCTGGAATGGATGGGATGGATTTTT | |
| | | CCAGGGAGCGGCAGCACTTACTACAACCAGAAATTTCAAGGTCGTGTGACAATGACCGTGGATAAAAGCAGCTCTACA | |
| | | GCTTACATGGAGCTTTCCCGCTTGAGGTCCGATGATACTGCCGTATATTATTGTGCCCGTGGTGACTCAGGTAGGGCC | |
| | | ATGGACTATTGGGGACAGGGCACCCTCGTGACCGTGTCCAGC | |
| | VL | GATATCCAGATGACACAATCCCCTTCATCCTTGAGCGCATCAGTTGGCGACAGGGTCACCATAACTTGTCAGGCTAGT | 302 |
| | | CAGGATATTAACAAGTACCTGGCTTGGTATCAACACAAGCCTGGAAAGGCCCCCAAATTGCTGATTCACTACACCTCT | |
| | | ACATTGGAAACTGGCGTACCCAGTCGCTTTTCTGGGAGTGGAAGCGGAACTGATTTCACTTTCACTATATCCAGTCTT | |
| | | CAGCCAGAAGATATCGCAACTTACTATTGTCTTCAGTATGATAACTTGCTTACTTTCGGAGGAGGGACCAAAGTTGAA | |
| | | ATCAAG | |
| 4540-033 | VH | CAGGTGCAGTTGGTCCAATCCGGGGCTGAGGTGAAGAAGCCTGGGGCCTCTGTTAAAGTTAGTTGCAAGGCATCAGGC | 303 |
| | | TACACCTTCGCTGACTACTACATCAACTGGGTTAGACAGGCCCCCGGGCAGGGGTTGGAGTGGATGGGTTGGATTTTT | |
| | | CCAGGATCAGGTTCAACATATTACGCACAAAAACTGCAAGGTAGAGTAACCATGACAACTGATACTAGCACCTCCACA | |
| | | GCCTATATGGAACTCCGCTCTCTCAGGAGTGACGATACAGCCGTTTATTACTGCGCCCGTGGGGATTCAGGCCGTGCA | |
| | | ATGGATTACTGGGGTCAAGGGACCCTCGTGACCGTAAGTTCA | |
| | VL | GATATCCAGATGACACAATCCCCTTCATCCTTGAGCGCATCAGTTGGCGACAGGGTCACCATAACTTGTCAGGCTAGT | 302 |
| | | CAGGATATTAACAAGTACCTGGCTTGGTATCAACACAAGCCTGGAAAGGCCCCCAAATTGCTGATTCACTACACCTCT | |
| | | ACATTGGAAACTGGCGTACCCAGTCGCTTTTCTGGGAGTGGAAGCGGAACTGATTTCACTTTCACTATATCCAGTCTT | |
| | | CAGCCAGAAGATATCGCAACTTACTATTGTCTTCAGTATGATAACTTGCTTACTTTCGGAGGAGGGACCAAAGTTGAA | |
| | | ATCAAG | |
| 4237 | VH | CAGGCGCACCTGAAGGAGTCAGGACCTGGCCTGGTGGCGCCCTCACAGAGCCTGTCCATCACCTGCACAGTCTCTGGT | 188 |
| | | TTCTCATTAACCGACTATGATGTACACTGGGTTCGCCAGTCTCCAGGAAAGGGTCTGGAGTGGCTGGGAGTGATATGG | |
| | | AATGATGGAAGCACAGACTATAATACAGCTTTCATATCTAGACTGACCATCAGCAAGGACAACTCCAAGAGCCAAGTT | |
| | | TTCTTTAAAATGAACAGTCTGCAAGCTGATGACACAGCCATATACTACTGTGCCAGAAATTGGTATGGTGGCTACTGG | |
| | | TTTGCTTACTGGGGCCAAGGGACTCTGGTCACTGTCTCTGCA | |
| | VL | GACATCCAGATGACTCAGTCTCCAGCCTCCCTATCTGCATCTGCGGGAGAAACTGTCACCATCACATGTCGATCAAGT | 189 |
| | | GAGAATATTTACAGTTATTTAGCATGGTATCAGCAGAAACAGGGAAAATCTCCTCAGCTCCTAGTCTATAATGCAAAT | |
| | | GCCTTAGCAGAAGGTGTGCCATCGAGGTTCAGTGGCAGTGGATCAGTCACACAGTTTTCTCTGAAGATCAACAGCCTG | |
| | | CAGCCTGAAGATTTTGGGAGTTATTACTGTCAACATCATTATGGTACTCCATTCACGTTCGGCTCGGGGACAAAGTTG | |
| | | GAAATAAAA | |
| 4439 | VH | GAGATCCAGCTGCAGCAGTCTGGAGCTGAACTGGTGAAGCCTGGGGCTTCAGTGAAGATATCCTGCAAGGCTTCTGAT | 297 |
| | | TACTCATTCACTGGCTACAACATGAACTGGGTGATGCAGAGCCATGGAAAGAGCCTTGAGTGGATTGGAAATATTCAT | |
| | | CCTTACTATGGTGGTACTAGCTTCAATCAGAAGTTCATGGGCAAGGCCACATTGACTGCAGACAAATCTTCCAGCACA | |
| | | GCCTACATGCAGCTCAACAGCCTGACATCTGAAGACTCTGCAGTCTATTACTGTGCAAGAGAGAGAAGTAACTTCCAT | |
| | | GCTCTGGACTACTGGGGTCAGGGAACCTCAGTCACCGTCTCCTCA | |
| | VL | GACATTGTGCTGACACAGTCTCCTGCTTCCTTAACTGTATCTCTGGGGCAGAGGGCCACCTTCTCATGCAGGGCCAGC | 298 |
| | | AAAAGTGTCAGTACATCTGGCTATAGTTATATGCACTGGTACCAACAGAAACCAGGACAGCCACCCAAACTCCTCATC | |
| | | TATTTTACATCCGACCTAGAACCTGGGGTCCCTGCCAGGTTCACTGGCAGTGGGTCTGGGACAGACTTCACCCTCAAC | |
| | | ATCCATCCTGTGGAGGAGGAGGATGCTGCAACCTATTACTGTCAGCACAGTAGGGAGCTTCCGTACCCCTTCGGAGGG | |
| | | GGGACCAAGTTGGAAATAAAA | |

TABLE 5
The Amino Acid Sequences of the Heavy Chain Variable Region (VH) and Light Chain Variable
Region (VL) of the Exemplary Humanized Anti-APRIL Antibodies Are Provided As Follows.

| | | SEQ |
|---|---|---|
| Antibody | | ID |
| Chain | Amino Acid Sequence | NO |
| >Hu_2218_VH01 | QVQLQESGPGLVKPSQTLSLTCTVSGYSITSGYYWNWIRQHPGKGLEWIGYISYDGYNNYNPSLKNRVTISVDTSKNQF | 190 |
| | SLKLSSVTAADTAVYYCARYYDYEDWYFGVWGQGTMVTVSS | |
| >Hu_2218_VH02 | QVQLQESGPGLVKPSQTLSLTCTVSGYSITSGYYWSWIRQHPGKGLEWIGYISYDGYTYYNPSLKSRVTISVDTSKNQF | 191 |
| | SLKLSSVTAADTAVYYCARYYDYEDWYFGVWGQGTMVTVSS | |
| >Hu_2218_VH03 | QVQLQESGPGLVKPSQTLSLTCTVSGYSITSGYYWNWIRQHPGKGLEWIGYISYDGYNNYNPSLKSRVTISRDTSKNQF | 192 |
| | SLKLSSVTAADTAVYYCARYYDYEDWYFGVWGQGTMVTVSS | |
| >Hu_2218_VH04 | QVQLQQWGAGLLKPSETLSLTCAVYGYSITSGYYWNWIRQPPGKGLEWIGYISYDGYNNYNPSLKNRVTISVDTSKNQF | 193 |
| | SLKLSSVTAADTAVYYCANYYDYEDWYFGVWGQGTTVTVSS | |
| >Hu_2218_VH05 | QVQLQQWGAGLVKPSETLSLTCAVYGYSITSGYYWNWIRQPPGKGLEWIGYISYDGYNNYNPSLKNRVTISRDTSKNQF | 194 |
| | SLKLSSVTAADTAVYYCANYYDYEDWYFGVWGQGTTVTVSS | |
| >Hu_2218_VH06 | QVQLQESGPGLVKPSETLSLTCTVSGYSITSGYYWNWIRQPPGKGLEWIGYISYDGYNNYNPSLKNRVTISVDTSKNQF | 195 |
| | SLKLSSVTAADTAVYYCANYYDYEDWYFGVWGQGTTVTVSS | |
| >Hu_2218_VH07 | QVQLQESGPGLVKPSETLSLTCTVSGYSITSGYYWNWIRQPPGKGLEWIGYISYDGYNNYNPSLKNRVTISRDTSKNQF | 196 |
| | SLKLSSVTAADTAVYYCANYYDYEDWYFGVWGQGTTVTVSS | |
| >Hu_2218_VH08 | QVQLQESGPGLMKPSETLSLTCSVSGYSITSGYYWSWIRKPPGKGLEYIGYVSYDGSTYYNPSLKSRVTISVDTSKNRF | 197 |
| | SLKLNSVTAADTAVYYCANYYDYEDWYFGYWGQGILVTVSS | |
| >Hu_2218_VH09 | QVQLQESGPGLMKPSETLSLTCSVSGYSITSGYYWNWIRKPPGKGLEWIGYISYDGYNNYNPSLKSRVTISRDTSKNRF | 198 |
| | SLKLNSVTAADTAVYYCANYYDYEDWYFGVWGQGILVTVSS | |
| >Hu_2218_VH10 | EVQLVESGGGLVQPGGSLRLSCAVSGYSITSGYYWNWIRQAPGKGLEWVASISYDGYNNYNPSVKGRITISRDDSKNTF | 199 |
| | YLQMNSLRAEDTAVYYCANYYDYEDWYFGVWGQGTLVTVSS | |
| >Hu_2218_VH11 | EVQLVESGGGLVQPGGSLRLSCAVSGYSITSGYYWNWIRQAPGKGLEWVAYISYDGYNNYNPSVKGRITISRDTSKNTF | 200 |
| | YLQMNSLRAEDTAVYYCANYYDYEDWYFGVWGQGTLVTVSS | |
| >Hu_2218_VH12 | QVQLVESGGGVVQPGRSLRLSCAASGYSITSGYYWNWVRQAPGKGLEWVAYISYDGYNNYNPSVKGRFTISRDNSKNTL | 201 |
| | YLQMNSLRAEDTAVYYCANYYDYEDWYFGVWGQGTMVTVSS | |
| >Hu_2218_VL01 | EIVLTQSPATLSLSPGERATLSCRASESVSIIGTNSIHWYQQKPGQAPRLLIYHASNLETGIPARFSGSGPGTDFTLTI | 202 |
| | SSLEPEDFAVYYCLQSRKIPYTFGQGTKLEIK | |
| >Hu_2218_VL02 | DIVLTQSPASLAVSPGQRATITCRASESVSIIGTNSIHWYQQKPGQPPKLLIYHASNLETGVPARFSGSGSGTDFTLTI | 203 |
| | NPVEANDTANYYCLQSRKIPYTFGGGTKLEIK | |
| >Hu_2218_VL03 | EIVMTQSPATLSVSPGERATLSCRASESVSIIGTNSLHWYQQKPGQAPRLLIYHASQSISGIPARFSGSGSGTEFTLTI | 204 |
| | SSLQSEDFAVYYCQQSRKIPYTFGGGTKVEIK | |
| >Hu_2218_VL04 | EIVMTQSPATLSVSPGERATLSCRASESVSIIGTNSIHWYQQKPGQAPRLLIYHASNLETGIPARFSGSGSRTEFTLTI | 205 |
| | SSLQSEDFAVYYCLQSRKIPYTFGGGTKVEIK | |
| >Hu_2218_VL05 | DIQLTQSPSSLSASVGDRVTITCRASESVSIIGTNSMNWYQQKPGKAPKLLIYHASYLESGVPSRFSGSGSGTDFTLTI | 206 |
| | SSLQPEDFATYYCLQSRKIPYTFGQGTKVEIK | |
| >Hu_2218_VL06 | DIQLTQSPSSLSASVGDRVTITCRASESVSIIGTNSMHWYQQKPGKAPKLLIYHASNLESGVPSRFSGSGSRTDFTLTI | 207 |
| | SSLQPEDFATYYCLQSRKIPYTFGQGTKVEIK | |
| >Hu_2218_VL07 | DIQMTQSPSSLSASVGDRVTITCRASESVSIIGTNSMHWYQQKPGKAPKLLIYHASNLESGVPSRFSGSGSGTDFTFTI | 208 |
| | SSLQPEDIATYYCLQSRKIPYTFGQGTKVEIK | |
| >Hu_2419_VH01 | QVQLVQSGAEVKKPGASVKVSCKASGYTFTDYTIHWVRQAPGQGLEWMGWIYPLRGSINYNEKFKDRVTSTRDTSISTA | 209 |
| | YMELSRLRSDDTVVYYCARHGAYYSNAFDYWGQGTLVTVSS | |
| >Hu_2419_VH02 | QVQLVQSGAEVKKPGASVKVSCKASGYTFTDYYMHWVRQAPGQGLEWMGRIYPLRGSTNYAQKFQGRVTSTRDTSISTA | 210 |
| | YMELSRLRSDDTVVYYCARHGAYYSNAFDYWGQGTLVTVSS | |
| >Hu_2419_VH03 | QVQLVQSGAEVKKPGASVKVSCKASGYTFTDYTIHWVRQAPGQGLEWMGWIYPLRGSINYNEKFKDRVTMTADTSSSTA | 211 |
| | YMELSRLRSDDTVVYYCARHGAYYSNAFDYWGQGTLVTVSS | |
| >Hu_2419_VH04 | QVQLVQSGAEVKKPGASVKVSCEASGYTFTDYTIHWVRQAPGKGLEWMGWIYPLRGSINYNEKFKDRVTMTADTSTDTA | 212 |
| | YMELSSLRSKDTAVYYCARHGAYYSNAFDYWGQGTLVTVSS | |
| >Hu_2419_VH05 | QVQLVQSGAEVVKPGASVKLSCKASGYTFTDYTMYWVKQAPGQGLEWIGEIYPLRGSINFNEKFKSKATLTVDKSASTA | 213 |
| | YMELSSLRSEDTAVYYCARHGAYYSNAFDYWGQGTLVTVSS | |
| >Hu_2419_VH06 | QVQLVQSGAEVVKPGASVKLSCKASGYTFTDYTMHWVKQAPGQGLEWIGEIYPLRGSINFNEKFKSKATLTVDKSASTA | 214 |
| | YMELSSLRSEDTAVYYCARHGAYYSNAFDYWGQGTLVTVSS | |
| >Hu 2419_VL01 | EIVLTQSPATLSLSPGERATLSCRASESVDNDGIRFMHWYQQKPGQAPRLLIYRASNLESGIPARFSGSGPGTDFTLTI | 215 |
| | SSLEPEDFAVYYCQQSNKDPYTFGQGTKLEIK | |
| >Hu_2419_VL02 | EIVLTQSPATLSLSPGERATLSCRASESVDNDGIRFMHWYQQKPGQAPRLLIYRASNLESGIPARFSGSGPGTDFTLTI | 216 |
| | SSLEPEDVAVYYCQQSNKDPYTFGQGTKLEIK | |
| >Hu_2419_VL03 | DIVMTQSPASLAVSLGERATINCRASESVDNDGIRFMHWYQQKPGQPPKLLIYRASNLESGVPDRFSGSGSRTDFTLTI | 217 |
| | SSLQAEDVAVYYCQQSNKDPYTFGGGTKVEIK | |
| >Hu_2419_VL04 | DIVMTQSPASLAVSLGERATINCRASESVDNDGIRFMHWYQQKPGQPPKLLIYRASNLESGVPDRFSGSGSRTDFTLTI | 218 |
| | NSLQAEDVAVYYCQQSNKDPYTFGGGTKVELK | |
| >Hu_2419_VL05 | DIVLTQSPATLSVSPGERATISCRASESVDNDGIRFMHWYQQKPGQPPKLLIYRASNLESGVPARFSGSGSRTDFTLTI | 219 |
| | SSVEPEDFATYYCQHSWEIPPTFGGGTKLEIK | |
| >hu_4035_VH01 | QVQLVESGGGVVQPGRSLRLSCAASGFSLTIYDVHWVRQAPGKGLEWVAVIWSDGSTDYNAAFISRFTISRDNSKNTLY | 220 |
| | LQMNSLRAEDTAVYYCARNWVDQAWFAYWGQGTLVTVSS | |
| >hu_4035_VH02 | QVQLVESGGGVVQPGRSLRLSCAASGFSLTIYDVHWVRQAPGKGLEWVGVIWSDGSTDYNAAFISRFTISKDNSKNTLY | 221 |
| | LQMNSLRAEDTAVYYCARNWVDQAWFAYWGQGTLVTVSS | |
| >hu_4035_VH03 | QVQLVESGGGVVQPGRSLRLSCAASGFSLTIYDVHWVRQAPGKGLEWVAVIWSDGSTDYADSVKGRFTISKDNSKNTLY | 222 |
| | LQMNSLRAEDTAVYYCARNWVDQAWFAYWGQGTLVTVSS | |
| >hu_4035_VH04 | QVQLVESGGGVVQPGRSLRLSCAVSGFSLTIYDVHWVRQAPGKGLEWVGVIWSDGSTDYADSVKGRFTISKDNSKNTVY | 223 |
| | LQMNSLRAEDTAVYYCARNWVDQAWFAYWGQGTLVTVSS | |
| >hu_4035_VH05 | QVQLQESGPGLVKPSETLSLTCTVSGFSLTIYDVHWIRQPPGKGLEWIGVIWSDGSTDYNAAFISRVTISVDTSKNQFS | 224 |
| | LKLSSVTAADTAVYYCARNWVDQAWFAYWGQGTLVTVSS | |
| >hu_4035_VH06 | QVQLQESGPGLVKPSETLSLTCTVSGFSLTIYDVHWVRQPPGKGLEWIGVIWSDGSTDYNPSLKSRVTISKDTSKNQVS | 225 |
| | LKLSSVTAADTAVYYCARNWVDQAWFAYWGQGTLVTVSS | |
| >hu_4035_VH07 | QVQLQESGPGLMKPSETLSLTCSVSGDSITIYDWHWIRQPPGKGLEWIGVVWSDGSTDYNPSLKSRVTISVDTSKNRFS | 226 |
| | LKLSSVTAADTAVYYCARNWVDQAWFAYWGQGTLVTVSS | |
| >hu_4035_VH07 | QVQLQESGPGLVKPSETLSLTCTVSGFSLTIYDVHWIRQPPGKGLEWIGVIWSDGSTDYNPSLKSRVTISKDNSKNQFS | 227 |
| | LKLSSVTAADTAVYYCARNWVDQAWFAYWGQGTLVTVSS | |
| >hu_4035_VH09 | QVQLQESGPGLVKPSETLSLTCTVSGFSLTIYDVHWIRQPPGKGLEWIGVIWSDGSTDYNPSLKSRVTISKDTSKNQVS | 262 |
| | LKLSSVTAADTAVYYCARNWVDQAWFAYWGQGTLVTVSS | |
| >hu_4035_VH10 | QVQLQESGPGLVKPSETLSLTCTVSGGSITIYDWHWVRQPPGKGLEWIGVIWSDGSTDYNPSLKSRVTISKDTSKNQFS | 263 |
| | LKLSSVTAADTAVYYCARNWVDQAWFAYWGQGTLVTVSS | |
| >hu_4035_VH11 | QVQLQESGPGLVKPSETLSLTCTVSGGSITIYDWHWVRQPPGKGLEWIGVIWSDGSTDYNPSLKSRVTISVDTSKNQFS | 264 |
| | LKLSSVTAADTAVYYCARNWVDQAWFAYWGQGTLVTVSS | |
| >hu_4035_VH12 | QVQLQESGPGLVKPSETLSLTCTVSGGSITIYDWHWIRQPPGKGLEWIGVIWSDGSTDYNPSLKSRVTISVDTSKNQFS | 265 |
| | LKLSSVTAADTAVYYCARNWVDQAWFAYWGQGTLVTVSS | |
| >hu_4035_VL01 | DIQMTQSPSSLSASVGDRVTITCRASKNIYSYLAWYQQKPGKAPKLLIYNAKTLPEGVPSRFSGSGSGTDFTLTISSLQ | 228 |
| | PEDFATYYCQHHYGTPLTFGQGTKLEIK | |
| >hu_4035_VL02 | DIQMTQSPSSLSASVGDRVTITCRASKNIYSYLAWYQQKPGKAPKLLVYNAKTLPEGVPSRFSGSGSGTDFTLTISSLQ | 229 |
| | PEDFATYYCQHHYGTPLTFGQGTKLEIK | |
| >hu_4035_VL03 | EIVLTQSPATLSLSPGERATLSCRASKNIYSYLAWYQQKPGQAPRLLIYNAKTRATGIPARFSGSGSGTDFTLTISSLE | 230 |
| | PEDFAVYYCQHHYGTPLTFGQGTKLEIK | |
| >hu_4035_VL04 | EIVLTQSPATLSLSPGERATLSCRASKNIYSYLAWYQQKPGQAPRLLVYNAKTLPEGIPARFSGSGSGTDFTLTISSLE | 231 |
| | PEDFAVYYCQHHYGTPLTFGQGTKLEIK | |
| >hu_4035_VL05 | EIVMTQSPATLSVSPGERATLSCRASKNIYSYLAWYQQKPGQAPRLLIYNAKTRATGIPARFSGSGSGTEFTLTISSLQ | 232 |
| | SEDFAVYYCQHHYGTPLTFGGGTKVEIK | |
| >hu_4035_VL06 | DIQLTQSPSFLSASVGDRVTITCRASKNIYSYLAWYQQKPGKAPKLLIYNAKSLQSGVPSRFSGSGSGTEFTLTISSLQ | 233 |
| | PEDFATYYCQHHYGTPLTFGGGTKLEIK | |
| >hu_4035_VL07 | DIQLTQSPSFLSASVGDRVTITCRASKNIYSYLAWYQQKPGKAPKLLIYNAKSLQSGVPSRFSGSGSGTEFTLTISSLQ | 234 |
| | PEDFATYYCQHHYGTPLTFGGGTKLEIK | |
| >hu_4237_VH01 | QLQLQESGSGLVKPSQTLSLTCAVSGFSLTDYDVHWVRQPPGKGLEWIGVIWNDGSTDYNPSLISRVTISKDNSKNQVS | 235 |
| | LKLSSVTAADTAVYYCARNWYGGYWFAYWGQGTLVTVSS | |
| >hu_4237_VH02 | QLQLQESGSGLVKPSQTLSLTCAVSGGSITDYDWHWVRQPPGKGLEWIGVIWNDGSTDYNPSLISRVTISVDNSKNQFS | 236 |
| | LKLSSVTAADTAVYYCARNWYGGYWFAYWGQGTLVTVSS | |
| >hu_4237_VH03 | QVQLVESGGGVVQPGRSLRLSCAASGFSFTDYDMHWVRQAPGKGLEWVAVIWNDGSTDYATSVIGRFTISRDNSKNTLY | 237 |
| | LQMNSLRAEDTAVYYCARNWYGGYWFAYWGQGTLVTVSS | |
| >hu_4237_VH04 | QVQLVESGGGVVQPGRSLRLSCAASGFSFTDYDMHWVRQAPGKGLEWVGVIWNDGSTDYATSVIGRFTISRDNSKNTLY | 238 |
| | LQMNSLRAEDTAVYYCARNWYGGYWFAYWGQGTLVTVSS | |
| >hu_4237_VH05 | QVQLQESGPGLMKPSETLSLTCSVSGGSITDYDWHWIRQPPGKGLEWIGVVWNDGSTDYNPSLKSRVTISVDTSKNRFS | 239 |
| | LKLNSVTAADTAVYYCARNWYGGYWFAYWGQGILVTVSS | |
| >hu_4237_VH06 | QVTLKESGPALVKPTQTLTLTCTFSGFSLTDYDVHWIRQPPGKALEWLAVIWNDGSTDYSPSLKSRLTITKDTSKNQVV | 240 |
| | LTMTNMDPVDTATYYCARNWYGGYWFAYWGQGTLVTVSS | |
| >hu_4237_VL01 | DIQMTQSPSSLSASVGDRVTITCRSSENIYSYLAWYQQKPGKAPKLLVYNANALAEGVPSRFSGSGSVTDFTLTISSLQ | 241 |
| | PEDFATYYCQHHYGTPFTFGQGTKLEIK | |
| >hu_4237_VL02 | DIQMTQSPSTLSASVGDRVTITCRSSENIYSYLAWYQQKPGKAPKLLVYNANALAEGVPSRFSGSGSVTEFTLTISSLQ | 242 |
| | PDDFATYYCQHHYGTPFTFGQGTKLEIK | |
| >hu_4237_VL03 | EIVMTQSPATLSVSPGERATLSCRASENIYSYLAWYQQKPGQAPRLLIYNANASAEGIPARFSGSGSGTEFTLTISSLQ | 243 |
| | SEDFAVYYCQQHYGTPFTFGGGTKVEIK | |
| >hu_4237_VL04 | DIQMTQSPSSLSASVGDRVTITCRASENIYSYLAWYQQKPGKAPKLLLYNANRLESGVPSRFSGSGSGTDFTLTISSLQ | 244 |
| | PEDFASYYCQHHYGTPFTFGSGTKLEIK | |
| >hu_4237_VL05 | DIQMTQSPSSLSASVGDRVTITCRASENIYSYLAWYQQKPGKAPKLLLYNANRLESGVPSRFSGSGSGTDYTLTISSLQ | 245 |
| | PEDFASYYCQHHYGTPFTFGSGTKLEIK | |
| >hu_3833_VH01 | QVQLVQSGAEVKKPGASVKVSCKASGYTFTNYWIGWVRQAPGQGLEWMGDIYPGGIGGGYTKYNEKFKGRVTMTADTST | 246 |
| | STAYMELRSLRSDDTAVYYCSRSETGRAMDYWGQGTLVTVSS | |
| >hu_3833_VH02 | QVQLVQSGAEVKKPGASVKVSCKASGYTFTNYWIGWVRQAPGQGLEWMGDIYPGGIGGGYTKYAQKLQGRVTMTADTST | 247 |
| | STAYMELRSLRSDDTAVYYCSRSETGRAMDYWGQGTLVTVSS | |
| >hu_3833_VH03 | QVQLVQSGAEVKKPGASVKVSCKASGYTFTNYWIGWVRQAPGQGLEWMGDIYPGGIGGGYTKYAQKFQGRVTMTADTST | 248 |
| | STAYMELSSLRSEDTAVYYCSRSETGRAMDYWGQGTLVTVSS | |
| >hu_3833_VH04 | QVQLVQSGAEVKKPGASVKVSCKASGYTFTNYWIGWVRQAPGQGLEWMGDIYPGGIGGGYTKYNEKFKGRVTMTADTST | 249 |
| | STAYMELSSLRSEDTAVYFCSRSETGRAMDYWGQGTLVTVSS | |
| >hu_3833_VH05 | QVQLVQSGAELKRPGASVKVSCKASGYTFTNYWMGWVKQAPGQGLEWMGDIYPGGIGGGYTNYAQKFKGKATMTADTSS | 250 |
| | STAYMQLSRLRSEDTAVYYCSRSETGRAMDYWGQGTLVTVSS | |
| >hu_3833_VL01 | DIQMTQSPSSLSASVGDRVTITCQASQDINKYLAWYQQKPGKAPKLLIHYTSTLKPGVPSRFSGSGSGTDFTFTISSLQ | 251 |
| | PEDIATYYCLQYDNLNTFGGGTKLEIK | |
| >hu_3833_VL02 | DIQMTQSPSSLSASVGDRVTITCKASQDINKYIAWYQQKPGKAPKLLIHYTSTLKPGVPSRFSGSGSGRDYTFTISSLQ | 252 |
| | PEDIATYYCLQYDNLNTFGGGTKLEIK | |
| >hu_3833_VL03 | DIQMTQSPSSLSASVGDRVTITCQASQDINKYLAWYQQKPGKAPKLLIYYTSTLETGVPSRFSGSGSGTDFTFSISSLQ | 253 |
| | PEDIATYYCLQYDNLNTFGGGTKLEIK | |
| >hu_4540_VH01 | QVQLVQSGAEVKKPGASVKVSCKASGYTFADYYINWVRQAPGKGLEWMGWIFPGSGSTYYNEKFKGRVTMTVDKSTSTA | 254 |
| | YMELSSLRSEDTAVYFCARGDSGRAMDYWGQGTLVTVSS | |
| >hu_4540_VH02 | QVQLVQSGAEVKKPGASVKVSCKASGYTFADYYMNWVRQAPGQGLEWMGWIFPGSGSTYYAEKFKGRVTSTRDTSISTA | 255 |
| | YMELSRLRSDDTVVYYCARGDSGRAMDYWGQGTLVTVSS | |
| >hu_4540_VH03 | QVQLVQSGAEVKKPGASVKVSCKASGYTFADYYINWVRQAPGQGLEWMGWIFPGSGSTYYAQKLQGRVTMTTDTSTSTA | 256 |
| | YMELRSLRSDDTAVYYCARGDSGRAMDYWGQGTLVTVSS | |
| >hu_4540_VH04 | QVQLVQSGAEVKKPGASVKVSCKASGYTFADYYINWVRQAPGQGLEWMGWIFPGSGSTYYAQKLQGRVTMTVDKSSSTA | 257 |
| | YMELRSLRSDDTAVYYCARGDSGRAMDYWGQGTLVTVSS | |
| >hu_4540_VH05 | QVQLVQSGAELKKPGASVKVSCKASGYTFADYYMNWVRQAPGQGLEWMGWIFPGSGSTYYNQKFQGRVTMTVDKSSSTA | 258 |
| | YMELSRLRSDDTAVYYCARGDSGRAMDYWGQGTLVTVSS | |
| >hu_4540_VL01 | DIQMTQSPSSLSASVGDRVTITCKASQDINKYIAWYQQKPGKAPKLLIHYTSTLQSGVPSRFSGSGSGTDFTFTISSLQ | 259 |
| | PEDIATYYCLQYDNLLTFGQGTKLEIK | |
| >hu_4540_VL02 | DIQMTQSPSSLSASVGDRVTITCKASQDINKYIAWYQHKPGKAPKLLIHYTSTLQSGVPSRFSGSGSGRDYTFTISSLQ | 260 |
| | PEDIATYYCLQYDNLLTFGQGTKLEIK | |
| >hu_4540_VL03 | DIQMTQSPSSLSASVGDRVTITCQASQDINKYLAWYQHKPGKAPKLLIHYTSTLETGVPSRFSGSGSGTDFTFTISSLQ | 261 |
| | PEDIATYYCLQYDNLLTFGGGTKVEIK | |

In an embodiment, the antibody molecule comprises one, two, or three CDRs of the VH region of an antibody molecule described herein, e.g., in Table 1 or 5 (e.g., any of monoclonal antibodies 2218, 2419, 2419-0105, 2419-0205, 2419-0206, 2419-0406, 2419-0605, 2419-0805, 2419-0806, 2419-1204, 2419-1205,2419-1210,2419-1305,2419-1306,2419-1310,2419-1406,2922,3327,3530,3525,3125, 2621, 4035, 4035-062, 3934, 3833, 3631, 3732, 4338, 4540, 4540-063, 4540-033, 4439, 4439, or 4237), using the Kabat or Chothia definitions of CDRs. In an embodiment, the antibody molecule comprises one, two, or three CDRs of the VL region of an antibody molecule described herein, e.g., in Table 1 or 5 (e.g., any of monoclonal antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, 4439, or 4237), using the Kabat or Chothia definitions of CDRs. In an embodiment, the antibody molecule comprises one or more (e.g., two or three) CDRs of the VH region and/or one or more (e.g., two or three) CDRs of the VL region of an antibody molecule described herein, e.g., in Table 1 or 5 (e.g., any of monoclonal antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, 4439, or 4237), using the Kabat or Chothia definitions of CDRs.

In an embodiment, the antibody molecule comprises one, two, or three VH CDRs described in Table 1 or 5. In an embodiment, the antibody molecule comprises one, two, or three VL CDRs described in Table 1 or 5. In an embodiment, the antibody molecule comprises one or more (e.g., two or three) VH CDRs and/or one or more (e.g., two or three) VL CDRs described in Table 1 or 5.

In an embodiment, the antibody molecule comprises one, two, three, or four frameworks of the VH region of an antibody molecule described in Table 1 or 5 (e.g., any of monoclonal antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, 4439, or 4237). In an embodiment, the antibody molecule comprises one, two, three, or four frameworks of the VL region of an antibody molecule described in Table 1 or 5 (e.g., any of monoclonal antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, 4439, or 4237). In an embodiment, the antibody molecule comprises one or more (e.g., two, three, or four) frameworks of the VH region and/or one or more (e.g., two, three, or four) frameworks of the VL region of an antibody molecule described in Table 1 or 5 (e.g., any of monoclonal antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237).

In an embodiment, the antibody molecule comprises a heavy chain variable region of an antibody molecule described herein, e.g., in Table 1 or 5 (e.g., any of monoclonal antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237). In an embodiment, the antibody molecule comprises a light chain variable region of an antibody molecule described herein, e.g., in Table 1 or 5 (e.g., any of monoclonal antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237). In an embodiment, the antibody molecule comprises a heavy chain variable region and a light chain variable region of an antibody molecule described herein, e.g., in Table 1 or 5 (e.g., any of monoclonal antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237).

In an embodiment, the antibody molecule comprises a heavy chain variable region having an amino acid sequence described in Table 1 or 5, or an amino acid sequence substantially identical thereof. In an embodiment, the antibody molecule comprises a light chain variable region having an amino acid sequence described in Table 1 or 5, or an amino acid sequence substantially identical thereof. In an embodiment, the antibody molecule comprises a heavy chain variable region having an amino acid sequence described in Table 1 or 5 (or an amino acid sequence substantially identical thereof) and a light chain variable region having an amino acid sequences described in Table 1 or 5 (or an amino acid sequence substantially identical thereof).

In an embodiment, the antibody molecule comprises a heavy chain variable region encoded by a nucleotide sequence described in Table 2, or a nucleotide sequence substantially identical thereof. In an embodiment, the antibody molecule comprises a light chain variable region encoded by a nucleotide sequence described in Table 2, or a nucleotide sequence substantially identical thereof. In an embodiment, the antibody molecule comprises a heavy chain variable region encoded by a nucleotide sequence described in Table 2 (or a nucleotide sequence substantially identical thereof) and a light chain variable region encoded by a nucleotide sequence described in Table 2 (or a nucleotide sequence substantially identical thereof).

In an embodiment, the antibody molecule further comprises a heavy chain constant region. In an embodiment, the heavy chain constant region is an IgG1 constant region, e.g., any of SEQ ID NOS: 320-322, or a functional portion thereof. In another embodiment, the heavy chain constant region is an IgG2 constant region, e.g., any of SEQ ID NOS: 323-326, or a functional portion thereof. In an embodiment, the antibody molecule further comprises a light chain constant region. In an embodiment, the antibody molecule further comprises a heavy chain constant region and a light chain constant region. In an embodiment, the antibody molecule comprises a heavy chain constant region, a light chain constant region, and heavy and light chain variable regions of an antibody molecule described in Table 1 or 5. In certain embodiments, the antibody molecule comprises a heavy chain constant region, a light chain constant region, and variable regions that comprise one, two, three, four, five, or six CDRs of an antibody molecule described in Table 1 or 5.

Exemplary heavy chain constant regions are described below.

Exemplary IgG1 constant regions
>IGHG1*01
(SEQ ID NO: 320)
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSP
>IGHG1*03
(SEQ ID NO: 321)
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKRVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSP
>IGHG1*04
(SEQ ID NO: 322)
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWESNGQPENNYKTTPPV
LDSDGSFFLYSKLTVDKSRWQQGNIFSCSVMHEALHNHYTQKSLSLSP
Exemplary IgG2 constant regions
>IGHG2*01
(SEQ ID NO: 323)
STKGPSVFPLAPCSRSTSESTAALGCLVKDYFPEPVTVSWNSGALTSGVHTFPAVLQSSGLYSLSSVVTV
PSSNFGTQTYTCNVDHKPSNTKVDKTVERKCCVECPPCPAPPVAGPSVFLFPPKPKDTLMISRTPEVTCV
VVDVSHEDPEVQFNWYVDGVEVHNAKTKPREEQFNSTFRVVSVLTVVHQDWLNGKEYKCKVSNKGLPAPI
EKTISKTKGQPREPQVYTLPPSREEMTKNQVSLTCLVKGFYPSDIAVEWESNGQPENNYKTTPPMLDSDG
SFFLYSKLTVDKSRWQQGNVFSCSVMHEALHNHYTQKSLSLSP
>IGHG2*02
(SEQ ID NO: 324)
STKGPSVFPLAPCSRSTSESTAALGCLVKDYFPEPVTVSWNSGALTSGVHTFPAVLQSSGLYSLSSVVTV
TSSNFGTQTYTCNVDHKPSNTKVDKTVERKCCVECPPCPAPPVAGPSVFLFPPKPKDTLMISRTPEVTCV
VVDVSHEDPEVQFNWYVDGMEVHNAKTKPREEQFNSTFRVVSVLTVVHQDWLNGKEYKCKVSNKGLPAPI
EKTISKTKGQPREPQVYTLPPSREEMTKNQVSLTCLVKGFYPSDIAVEWESNGQPENNYKTTPPMLDSDG
SFFLYSKLTVDKSRWQQGNVFSCSVMHEALHNHYTQKSLSLSP
>IGHG2*04
(SEQ ID NO: 325)
STKGPSVFPLAPCSRSTSESTAALGCLVKDYFPEPVTVSWNSGALTSGVHTFPAVLQSSGLYSLSSVVTV
PSSSLGTQTYTCNVDHKPSNTKVDKTVERKCCVECPPCPAPPVAGPSVFLFPPKPKDTLMISRTPEVTCV
VVDVSHEDPEVQFNWYVDGVEVHNAKTKPREEQFNSTFRVVSVLTVVHQDWLNGKEYKCKVSNKGLPAPI
EKTISKTKGQPREPQVYTLPPSREEMTKNQVSLTCLVKGFYPSDIAVEWESNGQPENNYKTTPPMLDSDG
SFFLYSKLTVDKSRWQQGNVFSCSVMHEALHNHYTQKSLSLSP
>IGHG2*06
(SEQ ID NO: 326)
STKGPSVFPLAPCSRSTSESTAALGCLVKDYFPEPVTVSWNSGALTSGVHTFPAVLQSSGLYSLSSVVTV
PSSNFGTQTYTCNVDHKPSNTKVDKTVERKCCVECPPCPAPPVAGPSVFLFPPKPKDTLMISRTPEVTCV
VVDVSHEDPEVQFNWYVDGVEVHNAKTKPREEQFNSTFRVVSVLTVVHQDWLNGKEYKCKVSNKGLPAPI
EKTISKTKGQPREPQVYTLPPSREEMTKNQVSLTCLVKGFYPSDISVEWESNGQPENNYKTTPPMLDSDG
SFFLYSKLTVDKSRWQQGNVFSCSVMHEALHNHYTQKSLSLSP

In an embodiment, the antibody molecule comprises one or both of:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 11; an HCDR2 comprising an amino acid sequence that differs by no more than 1, 2, or 3 amino acid residues from, or has at least 85, 90, 95, 99 or 100% homology with, the amino acid sequence of SEQ ID NO: 12; or an HCDR3 comprising an amino acid sequence that differs by no more than 1, 2, or 3 amino acid residues from, or has at least 85, 90, 95, 99 or 100% homology with, the amino acid sequence of SEQ ID NO: 13, or
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of SEQ ID NO: 280; an LCDR2 comprising an amino acid sequence that differs by no more than 1, 2, or 3 amino acid residues from, or has at least 85, 90, 95, 99 or 100% homology with, the amino acid sequence of the SEQ ID NO: 285; or an LCDR3 comprising an amino acid sequence that differs by no more than 1, 2, or 3 amino acid residues from, or has at least 85, 90, 95, 99 or 100% homology with, the amino acid sequence of SEQ ID NO: 16.

In an embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 11; an HCDR2 comprising the amino acid sequence of SEQ ID NO: 12; and an HCDR3 comprising the amino acid sequence of SEQ ID NO: 13, and
(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 280; an LCDR2 comprising the amino acid sequence of SEQ ID NO: 285; or an LCDR3 comprising the amino acid sequence of SEQ ID NO: 16.

In an embodiment, the antibody molecule comprises one or both of:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 17; an HCDR2 comprising an amino acid sequence that differs by no more than 1, 2, or 3 amino acid residues from, or has at least 85, 90, 95, 99 or 100% homology with, the amino acid sequence of SEQ ID NO: 282; or an HCDR3 comprising an amino acid sequence that differs by no more than 1, 2, or 3 amino acid residues from, or has at least 85, 90, 95, 99 or 100% homology with, the amino acid sequence of SEQ ID NO: 13, or
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 280; an LCDR2 comprising an amino acid sequence that differs by no more than 1, 2, or 3 amino acid residues from, or has at least 85, 90, 95, 99 or 100% homology with, the amino acid sequence of the SEQ ID NO: 285; or an LCDR3 comprising an amino acid sequence that differs by no more than 1, 2, or 3 amino acid residues from, or has at least 85, 90, 95, 99 or 100% homology with, the amino acid sequence of SEQ ID NO: 16.

In an embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 17; an HCDR2 comprising the amino acid sequence of SEQ ID NO: 282; and an HCDR3 comprising the amino acid sequence of SEQ ID NO: 13, and
(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 280; an LCDR2 comprising the amino acid sequence of SEQ ID NO: 285; or an LCDR3 comprising the amino acid sequence of SEQ ID NO: 16.

In an embodiment, the antibody molecule comprises a VH comprising the amino acid sequence of SEQ ID NO: 296. In an embodiment, the antibody molecule comprises a VL comprising the amino acid sequence of SEQ ID NO: 286. In an embodiment, the antibody molecule comprises a VH comprising the amino acid sequence of SEQ ID NO: 296 and a VL comprising the amino acid sequence of SEQ ID NO: 286.

In an embodiment, the antibody molecule comprises a VH encoded by a nucleic acid comprising the nucleotide sequence of SEQ ID NO: 313. In an embodiment, the antibody molecule comprises a VL encoded by a nucleic acid comprising the nucleotide sequence of SEQ ID NO: 306. In an embodiment, the antibody molecule comprises a VH encoded by a nucleic acid comprising the nucleotide sequence of SEQ ID NO: 313 and a VL encoded by a nucleic acid comprising the nucleotide sequence of SEQ ID NO: 306.

In an embodiment, the antibody molecule further comprises a heavy constant region of IgG2, e.g., any of SEQ ID NOS: 323-326.

In an embodiment, the antibody molecule comprises one or both of:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 11; an HCDR2 comprising an amino acid sequence that differs by no more than 1, 2, or 3 amino acid residues from, or has at least 85, 90, 95, 99 or 100% homology with, the amino acid sequence of SEQ ID NO: 12; or an HCDR3 comprising an amino acid sequence that differs by no more than 1, 2, or 3 amino acid residues from, or has at least 85, 90, 95, 99 or 100% homology with, the amino acid sequence of SEQ ID NO: 13, or
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of SEQ ID NO: 280; an LCDR2 comprising an amino acid sequence that differs by no more than 1, 2, or 3 amino acid residues from, or has at least 85, 90, 95, 99 or 100% homology with, the amino acid sequence of the SEQ ID NO: 285; or an LCDR3 comprising an amino acid sequence that differs by no more than 1, 2, or 3 amino acid residues from, or has at least 85, 90, 95, 99 or 100% homology with, the amino acid sequence of SEQ ID NO: 16.

In an embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 11; an HCDR2 comprising the amino acid sequence of SEQ ID NO: 12; and an HCDR3 comprising the amino acid sequence of SEQ ID NO: 13, and
(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 280; an LCDR2 comprising the amino acid sequence of SEQ ID NO: 285; or an LCDR3 comprising the amino acid sequence of SEQ ID NO: 16.

In an embodiment, the antibody molecule comprises one or both of:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 17; an HCDR2 comprising an amino acid sequence that differs by no more than 1, 2, or 3 amino acid residues from, or has at least 85, 90, 95, 99 or 100% homology with, the amino acid sequence of SEQ ID NO: 282; or an HCDR3 comprising an amino acid sequence that differs by no more than 1, 2, or 3 amino acid residues from, or has at least 85, 90, 95, 99 or 100% homology with, the amino acid sequence of SEQ ID NO: 13, or
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 280; an LCDR2 comprising an amino acid sequence that differs by no more than 1, 2, or 3 amino acid residues from, or has at least 85, 90, 95, 99 or 100% homology with, the amino acid sequence of the SEQ ID NO: 285; or an LCDR3 comprising an amino acid sequence that differs by no more than 1, 2, or 3 amino acid residues from, or has at least 85, 90, 95, 99 or 100% homology with, the amino acid sequence of SEQ ID NO: 16.

In an embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 17; an HCDR2 comprising the amino acid sequence of SEQ ID NO: 282; and an HCDR3 comprising the amino acid sequence of SEQ ID NO: 13, and
(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 280; an LCDR2 comprising the amino acid sequence of SEQ ID NO: 285; or an LCDR3 comprising the amino acid sequence of SEQ ID NO: 16.

In an embodiment, the antibody molecule comprises a VH comprising the amino acid sequence of SEQ ID NO: 289. In an embodiment, the antibody molecule comprises a VL comprising the amino acid sequence of SEQ ID NO: 286. In an embodiment, the antibody molecule comprises a VH comprising the amino acid sequence of SEQ ID NO: 289 and a VL comprising the amino acid sequence of SEQ ID NO: 286.

In an embodiment, the antibody molecule comprises a VH encoded by a nucleic acid comprising the nucleotide sequence of SEQ ID NO: 308. In an embodiment, the antibody molecule comprises a VL encoded by a nucleic acid comprising the nucleotide sequence of SEQ ID NO: 305. In an embodiment, the antibody molecule comprises a VH encoded by a nucleic acid comprising the nucleotide sequence of SEQ ID NO: 308 and a VL encoded by a nucleic acid comprising the nucleotide sequence of SEQ ID NO: 306.

In an embodiment, the antibody molecule further comprises a heavy constant region of IgG2, e.g., any of SEQ ID NOS: 323-326.

In an embodiment, the antibody molecule comprises one or both of:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 11; an HCDR2 comprising an amino acid sequence that differs by no more than 1, 2, or 3 amino acid residues from, or has at least 85, 90, 95, 99 or 100% homology with, the amino acid sequence of SEQ ID NO: 12; or an HCDR3 comprising an amino acid sequence that differs by no more than 1, 2, or 3 amino acid residues from, or has at least 85, 90, 95, 99 or 100% homology with, the amino acid sequence of SEQ ID NO: 13, or
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of SEQ ID NO: 280; an LCDR2 comprising an amino acid sequence that differs by no more than 1, 2, or 3 amino acid residues from, or has at least 85, 90, 95, 99 or 100% homology with, the amino acid sequence of the SEQ ID NO: 281; or an LCDR3 comprising an amino acid sequence that differs by no more than 1, 2, or 3 amino acid residues from, or has at least 85, 90, 95, 99 or 100% homology with, the amino acid sequence of SEQ ID NO: 16.

In an embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 11; an HCDR2 comprising the amino acid sequence of SEQ ID NO: 12; and an HCDR3 comprising the amino acid sequence of SEQ ID NO: 13, and
(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 280; an LCDR2 comprising the amino acid sequence of SEQ ID NO: 281; or an LCDR3 comprising the amino acid sequence of SEQ ID NO: 16.

In an embodiment, the antibody molecule comprises one or both of:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 17; an HCDR2 comprising an amino acid sequence that differs by no more than 1, 2, or 3 amino acid residues from, or has at least 85, 90, 95, 99 or 100% homology with, the amino acid sequence of SEQ ID NO: 282; or an HCDR3 comprising an amino acid sequence that differs by no more than 1, 2, or 3 amino acid residues from, or has at least 85, 90, 95, 99 or 100% homology with, the amino acid sequence of SEQ ID NO: 13, or
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 280; an LCDR2 comprising an amino acid sequence that differs by no more than 1, 2, or 3 amino acid residues from, or has at least 85, 90, 95, 99 or 100% homology with, the amino acid sequence of the SEQ ID NO: 281; or an LCDR3 comprising an amino acid sequence that differs by no more than 1, 2, or 3 amino acid residues from, or has at least 85, 90, 95, 99 or 100% homology with, the amino acid sequence of SEQ ID NO: 16.

In an embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 17; an HCDR2 comprising the amino acid sequence of SEQ ID NO: 282; and an HCDR3 comprising the amino acid sequence of SEQ ID NO: 13, and
(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 280; an LCDR2 comprising the amino acid sequence of SEQ ID NO: 281; or an LCDR3 comprising the amino acid sequence of SEQ ID NO: 16.

In an embodiment, the antibody molecule comprises a VH comprising the amino acid sequence of SEQ ID NO: 289. In an embodiment, the antibody molecule comprises a VL comprising the amino acid sequence of SEQ ID NO: 284. In an embodiment, the antibody molecule comprises a VH comprising the amino acid sequence of SEQ ID NO: 289 and a VL comprising the amino acid sequence of SEQ ID NO: 284.

In an embodiment, the antibody molecule comprises a VH encoded by a nucleic acid comprising the nucleotide sequence of SEQ ID NO: 308. In an embodiment, the antibody molecule comprises a VL encoded by a nucleic acid comprising the nucleotide sequence of SEQ ID NO: 305. In an embodiment, the antibody molecule comprises a VH encoded by a nucleic acid comprising the nucleotide sequence of SEQ ID NO: 308 and a VL encoded by a nucleic acid comprising the nucleotide sequence of SEQ ID NO: 305.

In an embodiment, the antibody molecule further comprises a heavy constant region of IgG2, e.g., any of SEQ ID NOS: 323-326.

In an embodiment, the antibody molecule comprises one or both of:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 93; an HCDR2 comprising an amino acid sequence that differs by no more than 1, 2, or 3 amino acid residues from, or has at least 85, 90, 95, 99 or 100% homology with, the amino acid sequence of SEQ ID NO: 94; or an HCDR3 comprising an amino acid sequence that differs by no more than 1, 2, or 3 amino acid residues from, or has at least 85, 90, 95, 99 or 100% homology with, the amino acid sequence of SEQ ID NO: 95, or
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of SEQ ID NO: 96; an LCDR2 comprising an amino acid sequence that differs by no more than 1, 2, or 3 amino acid residues from, or has at least 85, 90, 95, 99 or 100% homology with, the amino acid sequence of the SEQ ID NO: 97; or an LCDR3 comprising an amino acid sequence that differs by no more than 1, 2, or 3 amino acid residues from, or has at least 85, 90, 95, 99 or 100% homology with, the amino acid sequence of SEQ ID NO: 98.

In an embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 93; an HCDR2 comprising the amino acid sequence of SEQ ID NO: 94; and an HCDR3 comprising the amino acid sequence of SEQ ID NO: 95, and
(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 96; an LCDR2 comprising the amino acid sequence of SEQ ID NO: 97; or an LCDR3 comprising the amino acid sequence of SEQ ID NO: 98.

In an embodiment, the antibody molecule comprises one or both of:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 99; an HCDR2 comprising an amino acid sequence that differs by no more than 1, 2, or 3 amino acid residues from, or has at least 85, 90, 95, 99 or 100% homology with, the amino acid sequence of SEQ ID NO: 273; or an HCDR3 comprising an amino acid sequence that differs by no more than 1, 2, or 3 amino acid residues from, or has at least 85, 90, 95, 99 or 100% homology with, the amino acid sequence of SEQ ID NO: 95, or
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 96; an LCDR2 comprising an amino acid sequence that differs by no more than 1, 2, or 3 amino acid residues from, or has at least 85, 90, 95, 99 or 100% homology with, the amino acid sequence of the SEQ ID NO: 97; or an LCDR3 comprising an amino acid sequence that differs by no more than 1, 2, or 3 amino acid residues from, or has at least 85, 90, 95, 99 or 100% homology with, the amino acid sequence of SEQ ID NO: 98.

In an embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 99; an HCDR2 comprising the amino acid sequence of SEQ ID NO: 273; and an HCDR3 comprising the amino acid sequence of SEQ ID NO: 95, and
(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of SEQ ID NO: 96; an LCDR2 comprising the amino acid sequence of SEQ ID NO: 97; or an LCDR3 comprising the amino acid sequence of SEQ ID NO: 98.

In an embodiment, the antibody molecule comprises a VH comprising the amino acid sequence of SEQ ID NO: 225. In an embodiment, the antibody molecule comprises a VL comprising the amino acid sequence of SEQ ID NO: 229. In an embodiment, the antibody molecule comprises a VH comprising the amino acid sequence of SEQ ID NO: 225 and a VL comprising the amino acid sequence of SEQ ID NO: 229.

In an embodiment, the antibody molecule comprises a VH encoded by a nucleic acid comprising the nucleotide sequence of SEQ ID NO: 299. In an embodiment, the antibody molecule comprises a VL encoded by a nucleic acid comprising the nucleotide sequence of SEQ ID NO: 300. In an embodiment, the antibody molecule comprises a VH encoded by a nucleic acid comprising the nucleotide sequence of SEQ ID NO: 299 and a VL encoded by a nucleic acid comprising the nucleotide sequence of SEQ ID NO: 300.

In an embodiment, the antibody molecule further comprises a heavy chain constant region of IgG1, e.g., any of SEQ ID NOS: 320-322.

In an embodiment, the antibody molecule described herein has one or more (e.g., 2, 3, 4, 5, or all) of the following properties: (a) is a humanized antibody molecule; (b) binds to human APRIL at an EC₅₀ of 60 pM or less, as determined by ELISA; (c) inhibits binding of human APRIL to TACI, e.g., in vitro, at an IC₅₀ of 0.5 nM or less; (d) inhibits binding of human APRIL to BCMI, e.g., in vitro, at an IC₅₀ of 0.6 nM or less; (e) is an IgG2κ; or (f) has an Fc region engineered to reduce complement activation. In an embodiment, the antibody molecule comprises one or more (e.g., 2, 3, 4, 5, or all) CDRs, one or both of heavy chain variable region or light chain variable regions, or one or both of heavy chain or light chain, of any of antibody molecules 2419-1406, 2419-0205, or 2419-0206. In an embodiment, the antibody molecule is suitable for use in treating a disorder in kidney, e.g., IgA nephropathy. In another embodiment, the antibody molecule is suitable for use in treating a caner, e.g., a multiple myeloma.

In an embodiment, the antibody molecule described herein has one or more (e.g., 2, 3, 4, 5, or all) of the following properties: (a) is a humanized antibody molecule; (b) binds to human APRIL at an EC₅₀ of 50 pM or less, as determined by ELISA; (c) inhibits binding of human APRIL to TACI, e.g., in vitro, at an IC₅₀ of 0.3 nM or less; (d) inhibits binding of human APRIL to BCMA, e.g., in vitro, at an IC₅₀ of 0.2 nM or less; (e) is an IgG1κ; or (f) has higher BCMA neutralization activity, e.g., has an IC₅₀ of 0.1 nM or less. In an embodiment, the antibody molecule comprises one or more (e.g., 2, 3, 4, 5, or all) CDRs, one or both of heavy chain variable region or light chain variable regions, or one or both of heavy chain or light chain, of antibody molecule 4035-062. In an embodiment, the antibody molecule is suitable for use in treating a cancer or an autoimmune disorder.

The antibody molecules described herein can have several advantageous properties. For example, the antibody molecules can be used to effectively treat, prevent or diagnose a disorder associated with APRIL, e.g., a disorder described herein, e.g., IgA nephropathy.

In an embodiment, the antibody molecule is capable of binding, or substantially binding, to human APRIL and mouse APRIL. In an embodiment, the antibody molecule is capable of binding, or substantially binding, to human APRIL, but is not capable of binding, or substantially binding to mouse APRIL. In an embodiment, the antibody molecule binds to APRIL with high affinity, e.g., with a dissociation constant (K_{D}) of less than about 100 nM, typically about 10 nM, and more typically, about 10-0.001 nM, about 10-0.01 nM, about 10-0.01 nM, about 5-0.01 nM, about 3-0.05 nM, about 1-0.1 nM, or stronger, e.g., less than about 80, 70, 60, 50, 40, 30, 20, 10, 8, 6, 4, 3, 2, 1, 0.5, 0.2, 0.1, 0.05, 0.01, 0.005, or 0.001 nM. In an embodiment, the antibody molecule binds to APRIL with a K_{off} slower than 1×10⁻⁴, 5×10⁻⁵, or 1×10⁻⁵ s⁻¹. In an embodiment, the antibody molecule binds to APRIL with a Kₒₙ faster than 1×10⁴, 5×10⁴, 1×10⁵, or 5×10⁵ M⁻¹s⁻¹.

In an embodiment, the antibody molecule is capable of inhibiting, or substantially inhibiting, binding of human APRIL to TACI. In an embodiment, the antibody molecule is capable of inhibiting, or substantially inhibiting, binding of human APRIL to TACI. In an embodiment, the antibody molecule is capable of inhibiting, or substantially inhibiting, binding of human APRIL to BCMA. In an embodiment, the antibody molecule is capable of inhibiting, or substantially inhibiting, binding of human APRIL to TACI and BCMA. In an embodiment, the antibody molecule is capable of inhibiting, or substantially inhibiting, binding of human APRIL to TACI, but is not capable of inhibiting, or substantially inhibiting, binding of human APRIL to BCMA. In an embodiment, the antibody molecule is capable of inhibiting, or substantially inhibiting, binding of human APRIL to BCMA, but is not capable of inhibiting, or substantially inhibiting, binding of human APRIL to TACI.

In an embodiment, the antibody molecule inhibits binding of human APRIL to human TACI by 50% or more, e.g., 60% or more, 70% or more, 80% or more, 85% or more, 90% or more, 95% or more, 96% or more, 97% or more, 98% or more, 99% or more, or 100%, as determined by a method described herein (e.g., normalized to the no antibody control).

In an embodiment, the antibody molecule inhibits binding of human APRIL to human BCMA by 30% or more, e.g., 40% or more, 50% or more, 60% or more, 70% or more, 80% or more, 85% or more, 90% or more, 95% or more, 96% or more, 97% or more, 98% or more, 99% or more, or 100%, as determined by a method described herein (e.g., normalized to the no antibody control).

In an embodiment, the antibody molecule does not substantially inhibit binding of human APRIL to human BCMA, e.g., inhibits binding of human APRIL to human BCMA by less than 10%, as determined by a method described herein (e.g., normalized to the no antibody control).

In an embodiment, binding of the antibody molecule to APRIL (e.g., human APRIL) inhibits, or substantially inhibits, the binding of the CRD2 domain of TACI (e.g., human TACI) to APRIL (e.g., human APRIL). In another embodiment, binding of the antibody molecule to human APRIL, inhibits, or substantially inhibits, the binding of human TACI, to one or more, e.g., 2, 3, 4, 5, 6, 7, 8, 9, 10, 11, 12, 13, 14, 15, 16, 17, 18, 19, 20, 21, 22, 23, 24, 25, or all of the APRIL residues from Table 3. In yet another embodiment, binding of the antibody molecule to human APRIL, inhibits, or substantially inhibits, the binding of human TACI, to one or more, e.g., 2, 3, 4, 5, 6, 7, 8, 9, 10, or all of the human APRIL residues from Table 4. In still another embodiment, binding of the antibody molecule to human APRIL, inhibits, or substantially inhibits, the binding of human TACI, to one or more, e.g., 2, 3, 4, 5, 6, 7, 8, 9, 10, 11, 12, 13, 14, 15, 16, 17, or all of the human APRIL residues from Table 7. In still another embodiment, binding of the antibody molecule to human APRIL, inhibits, or substantially inhibits, the binding of human TACI, to one or more, e.g., 2, 3, 4, 5, 6, 7, 8, 9, 10, 11, 12, 13, 14, 15, 16, 17, 18, 19, 20, 21, 22, 23, 24, 25, or all of the human APRIL residues from Table 8 of International Application Publication No. WO2017/091683.

### Exemplary Anti-APRIL Antibody Molecules

This disclosure provides, at least in part, antibody molecules that bind to APRIL, e.g., human and/or mouse APRIL, and that comprise one or more functional and structural properties disclosed herein. In an embodiment, the antibody molecule binds to and/or reduces (e.g., inhibits, blocks or neutralizes) one or more activities of APRIL. In an embodiment, the antibody molecule binds to a region in APRIL that interacts with TACI (e.g., the CRD2 domain of TACI). In an embodiment, the antibody molecule is selected from Table 1 or 5. In an embodiment, the antibody molecule comprises one or more heavy chain variable regions and/or one or more light chain variable regions described in Table 1 or 5. In an embodiment, the antibody molecule comprises one or more heavy chain CDRs and/or one or more light chain CDRs described in Table 1 or 5. In an embodiment, nucleic acid molecules encoding the antibody molecules, expression vectors, host cells, compositions (e.g., pharmaceutical compositions), kits, containers, and methods for making the antibody molecules, are also provided. The antibody molecules disclosed herein can be used (alone or in combination with other agents or therapeutic modalities) to treat, prevent and/or diagnose disorders associated with APRIL, such as IgA nephropathy.

In an embodiment, the antibody molecule has one or more (e.g., 1, 2, 3, 4, 5, 6, 7, 8, 9, 10, 11, 12, 13, 14, 15, 16, 17, 18, 19, 20, 21, 22, or 23) of the following properties:
a) Binds to human APRIL with high affinity, e.g., with a dissociation constant (K_{D}) of less than about 100 nM, typically about 10 nM, and more typically, about 10-0.001 nM, about 10-0.01 nM, about 5-0.01 nM, about 3-0.05 nM, about 1-0.1 nM, or stronger, e.g., less than about 80, 70, 60, 50, 40, 30, 20, 10, 8, 6, 4, 3, 2, 1, 0.5, 0.2, 0.1, 0.05, 0.01, 0.005, or 0.001 nM,
b) Binds to mouse APRIL with high affinity, e.g., with a dissociation constant (K_{D}) of less than about 100 nM, typically about 10 nM, and more typically, about 10-0.001 nM, about 10-0.01 nM, about 5-0.01 nM, about 3-0.05 nM, about 1-0.1 nM, or stronger, e.g., less than about 80, 70, 60, 50, 40, 30, 20, 10, 8, 6, 4, 3, 2, 1, 0.5, 0.2, 0.1, 0.05, 0.01, 0.005, or 0.001 nM,
c) Does not bind to mouse APRIL, or binds mouse APRIL with low affinity, e.g., with a dissociation constant (K_{D}) of greater than about 500 nM, e.g., greater than about 1000 nM,
d) Does not bind, or binds with low affinity, e.g., with a dissociation constant (K_{D}) of greater than about 500 nM, e.g., greater than about 1000 nM, to one or more (e.g., 2, 3, 4, 5, 6, 7, 8, or more) cytokines from the TNF superfamily (TNFSF) other than APRIL (e.g., TNFα, CD40 (TNFSF4), FasL (TNFSF6), TRAIL (TNFSF10), RANKL (TNFSF11), Tweak (TNFSF12), BAFF (TNFSF13B), or LIGHT (TNFSF14)),
e) Reduces (e.g., inhibits, blocks, or neutralizes) one or more biological activities of APRIL (e.g., human APRIL, mouse APRIL, or both), in vitro, ex vivo, or in vivo,
f) Reduces (e.g., inhibits, blocks, or neutralizes) binding of human APRIL to TACI, e.g., at an IC₅₀ of about 50 nM or less, typically about 0.01-50 nM, 0.1-25 nM, 0.1-10 nM, 0.5-5 nM, or 1-5 nM, e.g., less than about 40, 30, 20, 10, 5, 1, 0.5, 0.2, 0.1, 0.05, or 0.01 nM, e.g., as determined by a method described herein,
g) Reduces (e.g., inhibits, blocks, or neutralizes) binding of mouse APRIL to TACI, e.g., at an IC₅₀ of about 100 nM or less, typically about 0.01-75 nM, 0.1-50 nM, 0.1-25 nM, 0.1-10 nM, 0.5-5 nM, or 1-5 nM, e.g., less than about 80, 60, 40, 20, 10, 5, 1, 0.5, 0.2, 0.1, 0.05, or 0.01 nM, e.g., as determined by a method described herein,
h) Reduces (e.g., inhibits, blocks, or neutralizes) binding of human APRIL to BMCA, e.g., at an IC₅₀ of about 50 nM or less, typically about 0.01-50 nM, 0.1-25 nM, 0.1-10 nM, 0.5-5 nM, or 1-5 nM, e.g., less than about 40, 30, 20, 10, 5, 1, 0.5, 0.2, 0.1, 0.05, or 0.01 nM, e.g., as determined by a method described herein,
i) Reduces (e.g., inhibits, blocks, or neutralizes) binding of mouse APRIL to BMCA, e.g., at an IC₅₀ of about 200 nM or less, typically about 0.01-200 nM, 0.1-150 nM, 0.1-100 nM, 0.1-50 nM, 0.1-25 nM, 0.1-10 nM, 0.5-5 nM, or 1-5 nM, e.g., less than about 150, 100, 50, 40, 30, 20, 10, 5, 1, 0.5, 0.2, 0.1, 0.05, or 0.01 nM, e.g., as determined by a method described herein,
j) Shows the same or similar binding affinity or specificity, or both, as a monoclonal antibody described in Table 1 or 5, e.g., any of monoclonal antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237,
k) Shows the same or similar binding affinity or specificity, or both, as an antibody molecule comprising a heavy chain variable region and/or light chain variable region described in Table 1 or 5, e.g., a heavy chain variable region and/or light chain variable region of any of monoclonal antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237,
l) Shows the same or similar binding affinity or specificity, or both, as an antibody molecule comprising one or more (e.g., two or three) heavy chain CDRs and/or one or more (e.g., two or three) light chain CDRs described in Table 1 or 5, e.g., one or more (e.g., two or three) heavy chain CDRs and/or one or more (two or three) light chain CDRs of any of monoclonal antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237,
m) Shows the same or similar binding affinity or specificity, or both, as an antibody molecule comprising an amino acid sequence shown in Table 1 or 5,
n) Shows the same or similar binding affinity or specificity, or both, as an antibody molecule comprising an amino acid sequence encoded by a nucleotide sequence shown in Table 2,
o) Has one or more structural properties of a monoclonal antibody chosen from Table 1 or 5, e.g., any of monoclonal antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237, or
p) Has one or more pharmacokinetic properties of a monoclonal antibody chosen from Table 1 or 5, e.g., any of monoclonal antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237.

In an embodiment, the anti-APRIL antibody molecule:
(i) binds, or substantially binds, to human APRIL;
(ii) binds, or substantially binds, to mouse APRIL;
(iii) inhibits, or substantially inhibits, binding of APRIL (e.g., human APRIL, mouse APRIL, or both) to TACI (e.g., human TACI, mouse TACI, or both); and
(iv) inhibits, or substantially inhibits, binding of APRIL (e.g., human APRIL, mouse APRIL, or both) to BCMA (e.g., human BCMA, mouse BCMA, or both).

In an embodiment, the antibody molecule is a synthetic antibody molecule. In an embodiment, the antibody molecule is an isolated antibody molecule.

In an embodiment, the antibody molecule binds, or substantially binds, to human APRIL at an EC₅₀ of 20 nM or less, e.g., 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 20 nM, e.g., between 0.01 nM and 20 nM, between 0.1 nM and 20 nM, between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein.

In an embodiment, the antibody molecule binds, or substantially binds, to mouse APRIL at an EC₅₀ of 100 nM or less, e.g., 80 nM or less, 60 nM or less, 40 nM or less, 20 nM or less, 10 nM or less, 9 nM or less 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 100 nM, e.g., between 0.001 nM and 50 nM, between 0.01 nM and 20 nM, between 0.1 nM and 10 nM, between 0.5 nM and 5 nM or between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein.

In an embodiment, the antibody molecule inhibits, or substantially inhibits, binding of APRIL (e.g., human APRIL, mouse APRIL, or both) to TACI (e.g., human TACI, mouse TACI, or both), at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein.

In an embodiment, the antibody molecule inhibits, or substantially inhibits, binding of APRIL (e.g., human APRIL, mouse APRIL, or both) to BCMA (e.g., human BCMA, mouse BCMA, or both), e.g., at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein.

In an embodiment, the antibody molecule is an IgG antibody molecule, e.g., comprising a heavy chain constant region of IgG, e.g., chosen from IgG1, IgG2 (e.g., IgG2a), IgG3, or IgG4, e.g., IgG2 or IgG4. In an embodiment, the antibody molecule is an IgG1 antibody molecule, e.g., having an IgG1 constant region described herein. In another embodiment, the antibody molecule is an IgG2 antibody molecule e.g., having an IgG2 constant region described herein. In an embodiment, the antibody molecule comprises a light chain constant region of kappa or lambda light chain.

In an embodiment, the antibody molecule comprises an Fc region. In an embodiment, the Fc region comprises one or more mutations located at the interface between the CH2 and CH3 domains (e.g., to increase the binding affinity to neonatal receptor FcRn and/or the half-life of the antibody molecule). In an embodiment, the Fc region comprises one or more mutations, e.g., one or more (e.g., 2, 3, 4, 5, 6 or all) mutations chosen from T250Q, M252Y, S254T, T256E, M428L, H433K, N434F, or any combination thereof, of IgG1. In an embodiment, the Fc region comprises one or more mutations at positions 233-236 or 322 of human IgG1 or IgG2, or one or more substitutions at positions 327, 330 or 331 of human IgG4 (e.g., to reduce complement-dependent cytotoxicity (CDC)). In an embodiment, the Fc region comprises one or more (e.g., 2, 3, 4, 5, 6, 7 or all) mutations chosen from E233P, L234V, L235A, G236, K322A, A327G, A330S, P331S, or any combination thereof.

In an embodiment, the antibody molecule is a humanized antibody molecule, e.g., comprising one or more framework regions derived from human framework germline sequence. In an embodiment, the antibody molecule comprises a heavy chain variable region (VH) described in Table 1 or 5. In an embodiment, the antibody molecule comprises a light chain variable region (VL) described in Table 1 or 5. In an embodiment, the antibody molecule comprises a heavy chain variable region (VH) and a light chain variable region (VL) described in Table 1 or 5. In an embodiment, the antibody molecule comprises one, two, or three CDRs of a heavy chain variable region (VH) described in Table 1 or 5. In an embodiment, the antibody molecule comprises one, two, or three CDRs of a light chain variable region (VL) described in Table 1 or 5. In an embodiment, the antibody molecule comprises one, two, or three CDRs of a heavy chain variable region (VH) described in Table 1 or 5, and one, two, or three CDRs of a light chain variable region (VL) described in Table 1 or 5. In an embodiment, the antibody molecule comprises two heavy chain variable regions and two light chain variable regions. In an embodiment, the antibody molecule is a Fab, F(ab′)2, Fv, Fd, or a single chain Fv fragment (scFv).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3530 (e.g., SEQ ID NO: 61); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3530 (e.g., SEQ ID NO: 62); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3530 (e.g., SEQ ID NO: 63).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3530 (e.g., SEQ ID NO: 67); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3530 (e.g., SEQ ID NO: 45); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3530 (e.g., SEQ ID NO: 46).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3530 (e.g., SEQ ID NO: 61); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3530 (e.g., SEQ ID NO: 62); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3530 (e.g., SEQ ID NO: 63), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3530 (e.g., SEQ ID NO: 67); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3530 (e.g., SEQ ID NO: 45); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3530 (e.g., SEQ ID NO: 46).

In an embodiment, the antibody molecule comprises: (i) a VH comprising: an HCDR1 comprising the amino acid sequence of the HCDR1 of monoclonal antibody 3530 (e.g., SEQ ID NO: 61); an HCDR2 comprising the amino acid sequence of the HCDR2 of monoclonal antibody 3530 (e.g., SEQ ID NO: 62); and an HCDR3 comprising the amino acid sequence of the HCDR3 of monoclonal antibody 3530 (e.g., SEQ ID NO: 63), and (ii) a VL comprising: an LCDR1 comprising the amino acid sequence of the LCDR1 of monoclonal antibody 3530 (e.g., SEQ ID NO: 67); an LCDR2 comprising the amino acid sequence of the LCDR2 of monoclonal antibody 3530 (e.g., SEQ ID NO: 45); and an LCDR3 comprising the amino acid sequence of the LCDR3 of monoclonal antibody 3530 (e.g., SEQ ID NO: 46).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3530 (e.g., SEQ ID NO: 64); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3530 (e.g., SEQ ID NO: 65); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3530 (e.g., SEQ ID NO: 63).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3530 (e.g., SEQ ID NO: 67); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3530 (e.g., SEQ ID NO: 45); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3530 (e.g., SEQ ID NO: 46).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3530 (e.g., SEQ ID NO: 64); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3530 (e.g., SEQ ID NO: 65); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3530 (e.g., SEQ ID NO: 63), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3530 (e.g., SEQ ID NO: 67); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3530 (e.g., SEQ ID NO: 45); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3530 (e.g., SEQ ID NO: 46).

In an embodiment, the antibody molecule comprises: (i) a VH comprising: an HCDR1 comprising the amino acid sequence of the HCDR1 of monoclonal antibody 3530 (e.g., SEQ ID NO: 64); an HCDR2 comprising the amino acid sequence of the HCDR2 of monoclonal antibody 3530 (e.g., SEQ ID NO: 65); and an HCDR3 comprising the amino acid sequence of the HCDR3 of monoclonal antibody 3530 (e.g., SEQ ID NO: 63), and (ii) a VL comprising: an LCDR1 comprising the amino acid sequence of the LCDR1 of monoclonal antibody 3530 (e.g., SEQ ID NO: 67); an LCDR2 comprising the amino acid sequence of the LCDR2 of monoclonal antibody 3530 (e.g., SEQ ID NO: 45); and an LCDR3 comprising the amino acid sequence of the LCDR3 of monoclonal antibody 3530 (e.g., SEQ ID NO: 46).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3530 (e.g., SEQ ID NO: 66). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3530 (e.g., SEQ ID NO: 70).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3530 (e.g., SEQ ID NO: 66); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3530 (e.g., SEQ ID NO: 70). In an embodiment, the antibody molecule comprises: (i) a VH comprising the amino acid sequence of the VH of monoclonal antibody 3530 (e.g., SEQ ID NO: 66); and (ii) a VL comprising the amino acid sequence of the VL of monoclonal antibody 3530 (e.g., SEQ ID NO: 70).

In an embodiment the antibody molecule is monoclonal antibody 3530. In an embodiment, the antibody molecule is a humanized monoclonal antibody 3530.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3525 (e.g., SEQ ID NO: 61); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3525 (e.g., SEQ ID NO: 62); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3525 (e.g., SEQ ID NO: 63).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3525 (e.g., SEQ ID NO: 44); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3525 (e.g., SEQ ID NO: 45); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3525 (e.g., SEQ ID NO: 46).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3525 (e.g., SEQ ID NO: 61); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3525 (e.g., SEQ ID NO: 62); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3525 (e.g., SEQ ID NO: 63), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3525 (e.g., SEQ ID NO: 44); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3525 (e.g., SEQ ID NO: 45); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3525 (e.g., SEQ ID NO: 46).

In an embodiment, the antibody molecule comprises: (i) a VH comprising three heavy chain complementarity determining regions (HCDR1, HCDR2, and HCDR3), wherein the heavy chain variable region comprises: an HCDR1 comprises the amino acid sequence of the HCDR1 of monoclonal antibody 3525 (e.g., SEQ ID NO: 61); an HCDR2 comprising the amino acid sequence of the HCDR2 of monoclonal antibody 3525 (e.g., SEQ ID NO: 62); and an HCDR3 comprising the amino acid sequence of the HCDR3 of monoclonal antibody 3525 (e.g., SEQ ID NO: 63), and (ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of monoclonal antibody 3525 (e.g., SEQ ID NO: 44); an LCDR2 comprising the amino acid sequence of the LCDR2 of monoclonal antibody 3525 (e.g., SEQ ID NO: 45); and an LCDR3 comprising the amino acid sequence of the LCDR3 of monoclonal antibody 3525 (e.g., SEQ ID NO: 46).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3525 (e.g., SEQ ID NO: 64); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3525 (e.g., SEQ ID NO: 65); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3525 (e.g., SEQ ID NO: 63).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3525 (e.g., SEQ ID NO: 44); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3525 (e.g., SEQ ID NO: 45); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3525 (e.g., SEQ ID NO: 46).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3525 (e.g., SEQ ID NO: 64); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3525 (e.g., SEQ ID NO: 65); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3525 (e.g., SEQ ID NO: 63), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3525 (e.g., SEQ ID NO: 44); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3525 (e.g., SEQ ID NO: 45); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3525 (e.g., SEQ ID NO: 46).

In an embodiment, the antibody molecule comprises: (i) a VH comprising: an HCDR1 comprises the amino acid sequence of the HCDR1 of monoclonal antibody 3525 (e.g., SEQ ID NO: 64); an HCDR2 comprising the amino acid sequence of the HCDR2 of monoclonal antibody 3525 (e.g., SEQ ID NO: 65); and an HCDR3 comprising the amino acid sequence of the HCDR3 of monoclonal antibody 3525 (e.g., SEQ ID NO: 63), and (ii) a VL comprising: an LCDR1 comprising the amino acid sequence of the LCDR1 of monoclonal antibody 3525 (e.g., SEQ ID NO: 44); an LCDR2 comprising the amino acid sequence of the LCDR2 of monoclonal antibody 3525 (e.g., SEQ ID NO: 45); and an LCDR3 comprising the amino acid sequence of the LCDR3 of monoclonal antibody 3525 (e.g., SEQ ID NO: 46).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3525 (e.g., SEQ ID NO: 66). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3525 (e.g., SEQ ID NO: 50).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3525 (e.g., SEQ ID NO: 66); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3525 (e.g., SEQ ID NO: 50). In an embodiment, the antibody molecule comprises: (i) a VH comprising the amino acid sequence of the VH of monoclonal antibody 3525 (e.g., SEQ ID NO: 66); and (ii) a VL comprising the amino acid sequence of the VL of monoclonal antibody 3525 (e.g., SEQ ID NO: 50).

In an embodiment the antibody molecule is monoclonal antibody 3525. In an embodiment, the antibody molecule is a humanized monoclonal antibody 3525.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3833 (e.g., SEQ ID NO: 113); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3833 (e.g., SEQ ID NO: 114); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3833 (e.g., SEQ ID NO: 115).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3833 (e.g., SEQ ID NO: 116); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3833 (e.g., SEQ ID NO: 117); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3833 (e.g., SEQ ID NO: 118).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3833 (e.g., SEQ ID NO: 113); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3833 (e.g., SEQ ID NO: 114); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3833 (e.g., SEQ ID NO: 115), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3833 (e.g., SEQ ID NO: 116); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3833 (e.g., SEQ ID NO: 117); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3833 (e.g., SEQ ID NO: 118).

In an embodiment, the antibody molecule comprises: (i) a VH comprising three heavy chain complementarity determining regions (HCDR1, HCDR2, and HCDR3), wherein the heavy chain variable region comprises: an HCDR1 comprises the amino acid sequence of the HCDR1 of monoclonal antibody 3833 (e.g., SEQ ID NO: 113); an HCDR2 comprising the amino acid sequence of the HCDR2 of monoclonal antibody 3833 (e.g., SEQ ID NO: 114); and an HCDR3 comprising the amino acid sequence of the HCDR3 of monoclonal antibody 3833 (e.g., SEQ ID NO: 115), and (ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of monoclonal antibody 3833 (e.g., SEQ ID NO: 116); an LCDR2 comprising the amino acid sequence of the LCDR2 of monoclonal antibody 3833 (e.g., SEQ ID NO: 117); and an LCDR3 comprising the amino acid sequence of the LCDR3 of monoclonal antibody 3833 (e.g., SEQ ID NO: 118).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3833 (e.g., SEQ ID NO: 119); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3833 (e.g., SEQ ID NO: 120); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3833 (e.g., SEQ ID NO: 115).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3833 (e.g., SEQ ID NO: 116); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3833 (e.g., SEQ ID NO: 117); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3833 (e.g., SEQ ID NO: 118).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3833 (e.g., SEQ ID NO: 119); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3833 (e.g., SEQ ID NO: 120); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3833 (e.g., SEQ ID NO: 115), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3833 (e.g., SEQ ID NO: 116); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3833 (e.g., SEQ ID NO: 117); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3833 (e.g., SEQ ID NO: 118).

In an embodiment, the antibody molecule comprises: (i) a VH comprising: an HCDR1 comprises the amino acid sequence of the HCDR1 of monoclonal antibody 3833 (e.g., SEQ ID NO: 119); an HCDR2 comprising the amino acid sequence of the HCDR2 of monoclonal antibody 3833 (e.g., SEQ ID NO: 120); and an HCDR3 comprising the amino acid sequence of the HCDR3 of monoclonal antibody 3833 (e.g., SEQ ID NO: 115), and (ii) a VL comprising: an LCDR1 comprising the amino acid sequence of the LCDR1 of monoclonal antibody 3833 (e.g., SEQ ID NO: 116); an LCDR2 comprising the amino acid sequence of the LCDR2 of monoclonal antibody 3833 (e.g., SEQ ID NO: 117); and an LCDR3 comprising the amino acid sequence of the LCDR3 of monoclonal antibody 3833 (e.g., SEQ ID NO: 118).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3833 (e.g., SEQ ID NO: 121). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3833 (e.g., SEQ ID NO: 122).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3833 (e.g., SEQ ID NO: 121); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3833 (e.g., SEQ ID NO: 122). In an embodiment, the antibody molecule comprises: (i) a VH comprising the amino acid sequence of the VH of monoclonal antibody 3833 (e.g., SEQ ID NO: 121); and (ii) a VL comprising the amino acid sequence of the VL of monoclonal antibody 3833 (e.g., SEQ ID NO: 122).

In an embodiment the antibody molecule is monoclonal antibody 3833. In an embodiment, monoclonal antibody 3833 is a humanized monoclonal antibody 3833. In an embodiment, the antibody molecule comprises a VH comprising the amino acid sequence of any of SEQ ID NO: 246-250, a VL comprising the amino acid sequence of any of SEQ ID NO: 251-253, or both.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3631 (e.g., SEQ ID NO: 123); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3631 (e.g., SEQ ID NO: 124); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3631 (e.g., SEQ ID NO: 125).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3631 (e.g., SEQ ID NO: 126); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3631 (e.g., SEQ ID NO: 127); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3631 (e.g., SEQ ID NO: 128).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3631 (e.g., SEQ ID NO: 123); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3631 (e.g., SEQ ID NO: 124); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3631 (e.g., SEQ ID NO: 125), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3631 (e.g., SEQ ID NO: 126); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3631 (e.g., SEQ ID NO: 127); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3631 (e.g., SEQ ID NO: 128).

In an embodiment, the antibody molecule comprises: (i) a VH comprising three heavy chain complementarity determining regions (HCDR1, HCDR2, and HCDR3), wherein the heavy chain variable region comprises: an HCDR1 comprises the amino acid sequence of the HCDR1 of monoclonal antibody 3631 (e.g., SEQ ID NO: 123); an HCDR2 comprising the amino acid sequence of the HCDR2 of monoclonal antibody 3631 (e.g., SEQ ID NO: 124); and an HCDR3 comprising the amino acid sequence of the HCDR3 of monoclonal antibody 3631 (e.g., SEQ ID NO: 125), and (ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of monoclonal antibody 3631 (e.g., SEQ ID NO: 126); an LCDR2 comprising the amino acid sequence of the LCDR2 of monoclonal antibody 3631 (e.g., SEQ ID NO: 127); and an LCDR3 comprising the amino acid sequence of the LCDR3 of monoclonal antibody 3631 (e.g., SEQ ID NO: 128).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3631 (e.g., SEQ ID NO: 129); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3631 (e.g., SEQ ID NO: 130); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3631 (e.g., SEQ ID NO: 125).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3631 (e.g., SEQ ID NO: 126); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3631 (e.g., SEQ ID NO: 127); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3631 (e.g., SEQ ID NO: 128).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3631 (e.g., SEQ ID NO: 129); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3631 (e.g., SEQ ID NO: 130); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3631 (e.g., SEQ ID NO: 125), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3631 (e.g., SEQ ID NO: 126); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3631 (e.g., SEQ ID NO: 127); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3631 (e.g., SEQ ID NO: 128).

In an embodiment, the antibody molecule comprises: (i) a VH comprising: an HCDR1 comprises the amino acid sequence of the HCDR1 of monoclonal antibody 3631 (e.g., SEQ ID NO: 129); an HCDR2 comprising the amino acid sequence of the HCDR2 of monoclonal antibody 3631 (e.g., SEQ ID NO: 130); and an HCDR3 comprising the amino acid sequence of the HCDR3 of monoclonal antibody 3631 (e.g., SEQ ID NO: 125), and (ii) a VL comprising: an LCDR1 comprising the amino acid sequence of the LCDR1 of monoclonal antibody 3631 (e.g., SEQ ID NO: 126); an LCDR2 comprising the amino acid sequence of the LCDR2 of monoclonal antibody 3631 (e.g., SEQ ID NO: 127); and an LCDR3 comprising the amino acid sequence of the LCDR3 of monoclonal antibody 3631 (e.g., SEQ ID NO: 128).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3631 (e.g., SEQ ID NO: 131). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3631 (e.g., SEQ ID NO: 132).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3631 (e.g., SEQ ID NO: 131); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3631 (e.g., SEQ ID NO: 132). In an embodiment, the antibody molecule comprises: (i) a VH comprising the amino acid sequence of the VH of monoclonal antibody 3631 (e.g., SEQ ID NO: 131); and (ii) a VL comprising the amino acid sequence of the VL of monoclonal antibody 3631 (e.g., SEQ ID NO: 132).

In an embodiment the antibody molecule is monoclonal antibody 3631. In an embodiment, the antibody molecule is a humanized monoclonal antibody 3631.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3732 (e.g., SEQ ID NO: 133); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3732 (e.g., SEQ ID NO: 134); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3732 (e.g., SEQ ID NO: 135).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3732 (e.g., SEQ ID NO: 136); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3732 (e.g., SEQ ID NO: 127); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3732 (e.g., SEQ ID NO: 137).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3732 (e.g., SEQ ID NO: 133); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3732 (e.g., SEQ ID NO: 134); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3732 (e.g., SEQ ID NO: 135), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3732 (e.g., SEQ ID NO: 136); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3732 (e.g., SEQ ID NO: 127); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3732 (e.g., SEQ ID NO: 137).

In an embodiment, the antibody molecule comprises: (i) a VH comprising three heavy chain complementarity determining regions (HCDR1, HCDR2, and HCDR3), wherein the heavy chain variable region comprises: an HCDR1 comprises the amino acid sequence of the HCDR1 of monoclonal antibody 3732 (e.g., SEQ ID NO: 133); an HCDR2 comprising the amino acid sequence of the HCDR2 of monoclonal antibody 3732 (e.g., SEQ ID NO: 134); and an HCDR3 comprising the amino acid sequence of the HCDR3 of monoclonal antibody 3732 (e.g., SEQ ID NO: 135), and (ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of monoclonal antibody 3732 (e.g., SEQ ID NO: 136); an LCDR2 comprising the amino acid sequence of the LCDR2 of monoclonal antibody 3732 (e.g., SEQ ID NO: 127); and an LCDR3 comprising the amino acid sequence of the LCDR3 of monoclonal antibody 3732 (e.g., SEQ ID NO: 137).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3732 (e.g., SEQ ID NO: 138); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3732 (e.g., SEQ ID NO: 139); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3732 (e.g., SEQ ID NO: 135).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3732 (e.g., SEQ ID NO: 136); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3732 (e.g., SEQ ID NO: 127); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3732 (e.g., SEQ ID NO: 137).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3732 (e.g., SEQ ID NO: 138); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3732 (e.g., SEQ ID NO: 139); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3732 (e.g., SEQ ID NO: 135), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3732 (e.g., SEQ ID NO: 136); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3732 (e.g., SEQ ID NO: 127); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3732 (e.g., SEQ ID NO: 137).

In an embodiment, the antibody molecule comprises: (i) a VH comprising: an HCDR1 comprises the amino acid sequence of the HCDR1 of monoclonal antibody 3732 (e.g., SEQ ID NO: 138); an HCDR2 comprising the amino acid sequence of the HCDR2 of monoclonal antibody 3732 (e.g., SEQ ID NO: 139); and an HCDR3 comprising the amino acid sequence of the HCDR3 of monoclonal antibody 3732 (e.g., SEQ ID NO: 135), and (ii) a VL comprising: an LCDR1 comprising the amino acid sequence of the LCDR1 of monoclonal antibody 3732 (e.g., SEQ ID NO: 136); an LCDR2 comprising the amino acid sequence of the LCDR2 of monoclonal antibody 3732 (e.g., SEQ ID NO: 127); and an LCDR3 comprising the amino acid sequence of the LCDR3 of monoclonal antibody 3732 (e.g., SEQ ID NO: 137).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3732 (e.g., SEQ ID NO: 140). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3732 (e.g., SEQ ID NO: 141).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3732 (e.g., SEQ ID NO: 140); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3732 (e.g., SEQ ID NO: 141). In an embodiment, the antibody molecule comprises: (i) a VH comprising the amino acid sequence of the VH of monoclonal antibody 3732 (e.g., SEQ ID NO: 140); and (ii) a VL comprising the amino acid sequence of the VL of monoclonal antibody 3732 (e.g., SEQ ID NO: 141).

In an embodiment the antibody molecule is monoclonal antibody 3732. In an embodiment, monoclonal antibody 3732 is a humanized monoclonal antibody 3732.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4540, 4540-063, or 4540-033 (e.g., SEQ ID NO: 154); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4540, 4540-063, or 4540-033 (e.g., SEQ ID NO: 155); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4540, 4540-063, or 4540-033 (e.g., SEQ ID NO: 156).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4540 (e.g., SEQ ID NO: 116), 4540-063 (e.g., SEQ ID NO: 274), or 4540-033 (e.g., SEQ ID NO: 274); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4540 (e.g., SEQ ID NO: 157), 4540-063 (e.g., SEQ ID NO: 275), or 4540-033 (e.g., SEQ ID NO: 275); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4540, 4540-063, or 4540-033 (e.g., SEQ ID NO: 158).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4540, 4540-063, or 4540-033 (e.g., SEQ ID NO: 154); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4540, 4540-063, or 4540-033 (e.g., SEQ ID NO: 155); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4540, 4540-063, or 4540-033 (e.g., SEQ ID NO: 156), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4540 (e.g., SEQ ID NO: 116), 4540-063 (e.g., SEQ ID NO: 274), or 4540-033 (e.g., SEQ ID NO: 274); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4540 (e.g., SEQ ID NO: 157), 4540-063 (e.g., SEQ ID NO: 275), or 4540-033 (e.g., SEQ ID NO: 275); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4540, 4540-063, or 4540-033 (e.g., SEQ ID NO: 158).

In an embodiment, the antibody molecule comprises: (i) a VH comprising three heavy chain complementarity determining regions (HCDR1, HCDR2, and HCDR3), wherein the heavy chain variable region comprises: an HCDR1 comprises the amino acid sequence of the HCDR1 of monoclonal antibody 4540, 4540-063, or 4540-033 (e.g., SEQ ID NO: 154); an HCDR2 comprising the amino acid sequence of the HCDR2 of monoclonal antibody 4540, 4540-063, or 4540-033 (e.g., SEQ ID NO: 155); and an HCDR3 comprising the amino acid sequence of the HCDR3 of monoclonal antibody 4540, 4540-063, or 4540-033 (e.g., SEQ ID NO: 156), and (ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of monoclonal antibody 4540 (e.g., SEQ ID NO: 116), 4540-063 (e.g., SEQ ID NO: 274), or 4540-033 (e.g., SEQ ID NO: 274); an LCDR2 comprising the amino acid sequence of the LCDR2 of monoclonal antibody 4540 (e.g., SEQ ID NO: 157), 4540-063 (e.g., SEQ ID NO: 275), or 4540-033 (e.g., SEQ ID NO: 275); and an LCDR3 comprising the amino acid sequence of the LCDR3 of monoclonal antibody 4540, 4540-063, or 4540-033 (e.g., SEQ ID NO: 158).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4540 (e.g., SEQ ID NO: 159), 4540-063 (e.g., SEQ ID NO: 276), or 4540-033 (e.g., SEQ ID NO: 159); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4540 (e.g., SEQ ID NO: 160), 4540-063 (e.g., SEQ ID NO: 277), or 4540-033 (e.g., SEQ ID NO: 278); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4540, 4540-063, or 4540-033 (e.g., SEQ ID NO: 156).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4540 (e.g., SEQ ID NO: 116), 4540-063 (e.g., SEQ ID NO: 274), or 4540-033 (e.g., SEQ ID NO: 274); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4540 (e.g., SEQ ID NO: 157), 4540-063 (e.g., SEQ ID NO: 275), or 4540-033 (e.g., SEQ ID NO: 275); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4540, 4540-063, or 4540-033 (e.g., SEQ ID NO: 158).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4540 (e.g., SEQ ID NO: 159), 4540-063 (e.g., SEQ ID NO: 276), or 4540-033 (e.g., SEQ ID NO: 159); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4540 (e.g., SEQ ID NO: 160), 4540-063 (e.g., SEQ ID NO: 277), or 4540-033 (e.g., SEQ ID NO: 278); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4540, 4540-063, or 4540-033 (e.g., SEQ ID NO: 156), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4540 (e.g., SEQ ID NO: 116), 4540-063 (e.g., SEQ ID NO: 274), or 4540-033 (e.g., SEQ ID NO: 274); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4540 (e.g., SEQ ID NO: 157), 4540-063 (e.g., SEQ ID NO: 275), or 4540-033 (e.g., SEQ ID NO: 275); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4540, 4540-063, or 4540-033 (e.g., SEQ ID NO: 158).

In an embodiment, the antibody molecule comprises: (i) a VH comprising one, two, or all of the following: an HCDR1 comprising the amino acid sequence of the HCDR1 of monoclonal antibody 4540 (e.g., SEQ ID NO: 159), 4540-063 (e.g., SEQ ID NO: 276), or 4540-033 (e.g., SEQ ID NO: 159); an HCDR2 comprising the amino acid sequence of the HCDR2 of monoclonal antibody 4540 (e.g., SEQ ID NO: 160), 4540-063 (e.g., SEQ ID NO: 277), or 4540-033 (e.g., SEQ ID NO: 278); or an HCDR3 comprising the amino acid sequence of the HCDR3 of monoclonal antibody 4540, 4540-063, or 4540-033 (e.g., SEQ ID NO: 156), and (ii) a VL comprising one, two, or all of the following: an LCDR1 comprising the amino acid sequence of the LCDR1 of monoclonal antibody 4540 (e.g., SEQ ID NO: 116), 4540-063 (e.g., SEQ ID NO: 274), or 4540-033 (e.g., SEQ ID NO: 274); an LCDR2 comprising the amino acid sequence of the LCDR2 of monoclonal antibody 4540 (e.g., SEQ ID NO: 157), 4540-063 (e.g., SEQ ID NO: 275), or 4540-033 (e.g., SEQ ID NO: 275); or an LCDR3 comprising the amino acid sequence of the LCDR3 of monoclonal antibody 4540, 4540-063, or 4540-033 (e.g., SEQ ID NO: 158).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 4540 (e.g., SEQ ID NO: 161), 4540-063 (e.g., SEQ ID NO: 258), or 4540-033 (e.g., SEQ ID NO: 256). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 4540 (e.g., SEQ ID NO: 162), 4540-063 (e.g., SEQ ID NO: 261), or 4540-033 (e.g., SEQ ID NO: 261).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 4540 (e.g., SEQ ID NO: 161), 4540-063 (e.g., SEQ ID NO: 258), or 4540-033 (e.g., SEQ ID NO: 256); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 4540 (e.g., SEQ ID NO: 162), 4540-063 (e.g., SEQ ID NO: 261), or 4540-033 (e.g., SEQ ID NO: 261). In an embodiment, the antibody molecule comprises: (i) a VH comprising the amino acid sequence of the VH of monoclonal antibody 4540 (e.g., SEQ ID NO: 161), 4540-063 (e.g., SEQ ID NO: 258), or 4540-033 (e.g., SEQ ID NO: 256); and (ii) a VL comprising the amino acid sequence of the VL of monoclonal antibody 4540 (e.g., SEQ ID NO: 162), 4540-063 (e.g., SEQ ID NO: 261), or 4540-033 (e.g., SEQ ID NO: 261).

In an embodiment, the antibody molecule is monoclonal antibody 4540, 4540-063, or 4540-033. In an embodiment, monoclonal antibody 4540 is a humanized monoclonal antibody 4540 (e.g., antibodies 4540-063 or 4540-033). In an embodiment, the antibody molecule comprises a VH comprising the amino acid sequence of any of SEQ ID NO: 254-258, a VL comprising the amino acid sequence of any of SEQ ID NO: 259-261, or both.

In an embodiment, the antibody molecule binds, or substantially binds, to human APRIL. In an embodiment, the antibody molecule binds, or substantially binds, to human APRIL and mouse APRIL. In an embodiment, the antibody molecule binds, or substantially binds to, human APRIL, but does not bind to mouse APRIL, or binds to mouse APRIL with low affinity.

In an embodiment, the antibody molecule binds, or substantially binds, to human APRIL at an EC₅₀ of 20 nM or less, e.g., 10 nM or less, 9 nM or less or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 20 nM, e.g., between 0.01 nM and 20 nM, between 0.1 nM and 20 nM, e.g., between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein.

In an embodiment, the antibody molecule binds, or substantially binds, to mouse APRIL at an EC₅₀ of 100 nM or less, e.g., 80 nM or less, 60 nM or less, 40 nM or less, 20 nM or less, e.g., 10 nM or less, e.g., 9 nM or less 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 100 nM, e.g., between 0.001 nM and 50 nM, between 0.01 nM and 20 nM, between 0.1 nM and 20 nM, e.g., between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein.

In an embodiment, the antibody molecule does not bind to mouse APRIL, or binds to mouse APRIL with low affinity, e.g., at an EC₅₀ of 1000 nM or more, e.g., 2000 nM or more, e.g., as determined by a method described herein.

In an embodiment, the antibody molecule inhibits, or substantially inhibits, binding of APRIL (e.g., human APRIL, mouse APRIL, or both) to TACI (e.g., human TACI, mouse TACI, or both). In an embodiment, the antibody molecule inhibits, or substantially inhibits, binding of APRIL (e.g., human APRIL, mouse APRIL, or both) to TACI (e.g., human TACI, mouse TACI, or both), at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein.

In an embodiment, binding of the antibody molecule to APRIL (e.g., human APRIL) inhibits, or substantially inhibits, the binding of the CRD2 domain of TACI (e.g., human TACI) to APRIL (e.g., human APRIL). In an embodiment, binding of the antibody molecule to human APRIL inhibits, or substantially inhibits, the binding of human TACI, to one or more, e.g., 2, 3, 4, 5, 6, 7, 8, 9, 10, 11, 12, 13, 14, 15, 16, 17, 18, 19, 20, 21, 22, 23, 24, 25, or all, of the human APRIL residues from Table 3. In an embodiment, binding of the antibody molecule to human APRIL, inhibits, or substantially inhibits, the binding of human TACI to one or more, e.g., 2, 3, 4, 5, 6, 7, 8, 9, 10, or all, of the human APRIL residues from Table 4. In an embodiment, binding of the antibody molecule to human APRIL inhibits, or substantially inhibits, the binding of human TACI, to one or more, e.g., 2, 3, 4, 5, 6, 7, 8, 9, 10, 11, 12, 13, 14, 15, 16, 17, or all, of the human APRIL residues from Table 7. In an embodiment, binding of the antibody molecule to human APRIL inhibits, or substantially inhibits, the binding of human TACI, to one or more, e.g., 2, 3, 4, 5, 6, 7, 8, 9, 10, 11, 12, 13, 14, 15, 16, 17, 18, 19, 20, 21, 22, 23, 24, 25, or all, of the human APRIL residues from Table 8 of International Application Publication No. WO2017/091683. In another embodiment, binding of the antibody molecule to human APRIL inhibits, or substantially inhibits, the binding of human BCMA, to one or more, e.g., 2, 3, 4, 5, 6, 7, 8, 9, 10, 11, 12, 13, 14, 15, 16, 17, 18, 19, 20, 21, 22, 23, 24, 25, or all, of the human APRIL residues from Table 8 of International Application Publication No. WO2017/091683.

In an embodiment, the antibody molecule inhibits, or substantially inhibits, binding of APRIL (e.g., human APRIL, mouse APRIL, or both) to BCMA (e.g., human BCMA, mouse BCMA, or both). In an embodiment, the antibody molecule inhibits, or substantially inhibits, binding of APRIL (e.g., human APRIL, mouse APRIL, or both) to BCMA (e.g., human BCMA, mouse BCMA, or both), e.g., at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein.

In an embodiment, the antibody molecule is a synthetic antibody molecule. In an embodiment, the antibody molecule is an isolated antibody molecule. In an embodiment, the antibody molecule is an IgG antibody molecule, e.g., comprising a heavy chain constant region of IgG, e.g., chosen from IgG1, IgG2 (e.g., IgG2a), IgG3, or IgG4, e.g., IgG2 or IgG4. In an embodiment, the antibody molecule is an IgG1 antibody molecule. In an embodiment, the antibody molecule is an IgG2 antibody molecule. In an embodiment, the antibody molecule comprises a light chain constant region of kappa or lambda light chain.

In an embodiment, the antibody molecule comprises an Fc region. In an embodiment, the Fc region comprises one or more mutations located at the interface between the CH2 and CH3 domains (e.g., to increase the binding affinity to neonatal receptor FcRn and/or the half-life of the antibody molecule).

In an embodiment, the Fc region comprises one or more mutations, e.g., one or more (e.g., 2, 3, 4, 6 or all) mutations chosen from T250Q, M252Y, S254T, T256E, M428L, H433K, N434F, or any combination thereof, of IgG1. In an embodiment, the Fc region comprises one or more mutations at positions 233-236 or 322 of human IgG1 or IgG2, or one or more substitutions at positions 327, 330 or 331 of human IgG4 (e.g., to reduce complement-dependent cytotoxicity (CDC)). In an embodiment, the Fc region comprises one or more (e.g., 2, 3, 4, 6 7 or all) mutations chosen from E233P, L234V, L235A, G236, K322A, A327G, A330S, P331S, or any combination thereof.

In an embodiment, the antibody molecule is a humanized antibody molecule, e.g., as described in Table 1 or 5, e.g., comprising one or more framework regions derived from human framework germline sequence.

In an embodiment, the antibody molecule comprises two heavy chain variable regions and two light chain variable regions. In an embodiment, the antibody molecule is a Fab, F(ab′)2, Fv, Fd, or a single chain Fv fragment (scFv).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 2218 (e.g., SEQ ID NO: 1); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 2218 (e.g., SEQ ID NO: 2); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 2218 (e.g., SEQ ID NO: 3).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 2218 (e.g., SEQ ID NO: 4); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 2218 (e.g., SEQ ID NO: 5); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 2218 (e.g., SEQ ID NO: 6).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 2218 (e.g., SEQ ID NO: 1); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 2218 (e.g., SEQ ID NO: 2); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 2218 (e.g., SEQ ID NO: 3), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 2218 (e.g., SEQ ID NO: 4); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 2218 (e.g., SEQ ID NO: 5); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 2218 (e.g., SEQ ID NO: 6).

In an embodiment, the antibody molecule comprises: (i) a VH comprising: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 2218 (e.g., SEQ ID NO: 1); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 2218 (e.g., SEQ ID NO: 2); and an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 2218 (e.g., SEQ ID NO: 3), and (ii) a VL comprising: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 2218 (e.g., SEQ ID NO: 4); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 2218 (e.g., SEQ ID NO: 5); and an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 2218 (e.g., SEQ ID NO: 6).

In an embodiment, the antibody molecule comprises a VH comprising one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 2218 (e.g., SEQ ID NO: 7); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 2218 (e.g., SEQ ID NO: 8); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 2218 (e.g., SEQ ID NO: 3).

In an embodiment, the antibody molecule comprises a VL comprising one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 2218 (e.g., SEQ ID NO: 4); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 2218 (e.g., SEQ ID NO: 5); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 2218 (e.g., SEQ ID NO: 6).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 2218 (e.g., SEQ ID NO: 7); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 2218 (e.g., SEQ ID NO: 8); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 2218 (e.g., SEQ ID NO: 3), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 2218 (e.g., SEQ ID NO: 4); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 2218 (e.g., SEQ ID NO: 5); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 2218 (e.g., SEQ ID NO: 6).

In an embodiment, the antibody molecule comprises: (i) a VH comprising: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 2218 (e.g., SEQ ID NO: 7); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 2218 (e.g., SEQ ID NO: 8); and an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 2218 (e.g., SEQ ID NO: 3), and (ii) a VL comprising: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 2218 (e.g., SEQ ID NO: 4); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 2218 (e.g., SEQ ID NO: 5); and an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 2218 (e.g., SEQ ID NO: 6).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 2218 (e.g., SEQ ID NO: 9). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 2218 (e.g., SEQ ID NO: 10).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 2218 (e.g., SEQ ID NO: 9); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 2218 (e.g., SEQ ID NO: 10). In an embodiment, the antibody molecule comprises: (i) a VH comprising the amino acid sequence of the VH of monoclonal antibody 2218 (e.g., SEQ ID NO: 9); and (ii) a VL comprising the amino acid sequence of the VL of monoclonal antibody 2218 (e.g., SEQ ID NO: 10).

In an embodiment the antibody molecule is monoclonal antibody 2218. In an embodiment, monoclonal antibody 2218 is a humanized monoclonal antibody 2218. In an embodiment, the antibody molecule comprises a VH comprising the amino acid sequence of any of SEQ ID NO: 190-201, a VL comprising the amino acid sequence of any of SEQ ID NO: 202-208, or both.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 2419 (e.g., SEQ ID NO: 11) or a 2419-related antibody; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 2419 (e.g., SEQ ID NO: 12) or a 2419-related antibody; or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 2419 (e.g., SEQ ID NO: 13) or a 2419-related antibody.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 2419 (e.g., SEQ ID NO: 14) or a 2419-related antibody; (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 2419 (e.g., SEQ ID NO: 15) or a 2419-related antibody; or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 2419 (e.g., SEQ ID NO: 16) or a 2419-related antibody.

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 2419 (e.g., SEQ ID NO: 11) or a 2419-related antibody; an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 2419 (e.g., SEQ ID NO: 12) or a 2419-related antibody; or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 2419 (e.g., SEQ ID NO: 13) or a 2419-related antibody, and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 2419 (e.g., SEQ ID NO: 14) or a 2419-related antibody; an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 2419 (e.g., SEQ ID NO: 15) or a 2419-related antibody; or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 2419 (e.g., SEQ ID NO: 16) or a 2419-related antibody.

In an embodiment, the antibody molecule comprises: (i) a VH comprising: an HCDR1 comprising the amino acid sequence of the HCDR1 of monoclonal antibody 2419 (e.g., SEQ ID NO: 11) or a 2419-related antibody; an HCDR2 comprising the amino acid sequence of the HCDR2 of monoclonal antibody 2419 (e.g., SEQ ID NO: 12) or a 2419-related antibody; or an HCDR3 comprising the amino acid sequence of the HCDR3 of monoclonal antibody 2419 (e.g., SEQ ID NO: 13) or a 2419-related antibody, and (ii) a VL comprising: an LCDR1 comprising the amino acid sequence of the LCDR1 of monoclonal antibody 2419 (e.g., SEQ ID NO: 14) or a 2419-related antibody; an LCDR2 comprising the amino acid sequence of the LCDR2 of monoclonal antibody 2419 (e.g., SEQ ID NO: 15) or a 2419-related antibody; and an LCDR3 comprising the amino acid sequence of the LCDR3 of monoclonal antibody 2419 (e.g., SEQ ID NO: 16) or a 2419-related antibody.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 2419 (e.g., SEQ ID NO: 17) or a 2419-related antibody; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 2419 (e.g., SEQ ID NO: 18) or a 2419-related antibody; or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 2419 (e.g., SEQ ID NO: 13) or a 2419-related antibody.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 2419 (e.g., SEQ ID NO: 14) or a 2419-related antibody; (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 2419 (e.g., SEQ ID NO: 15) or a 2419-related antibody; or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 2419 (e.g., SEQ ID NO: 16) or a 2419-related antibody.

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 2419 (e.g., SEQ ID NO: 17) or a 2419-related antibody; an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 2419 (e.g., SEQ ID NO: 18) or a 2419-related antibody; or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 2419 (e.g., SEQ ID NO: 13) or a 2419-related antibody, and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 2419 (e.g., SEQ ID NO: 14) or a 2419-related antibody; an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 2419 (e.g., SEQ ID NO: 15) or a 2419-related antibody; or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 2419 (e.g., SEQ ID NO: 16) or a 2419-related antibody.

In an embodiment, the antibody molecule comprises: (i) a VH comprising: an HCDR1 comprising the amino acid sequence of the HCDR1 of monoclonal antibody 2419 (e.g., SEQ ID NO: 17) or a 2419-related antibody; an HCDR2 comprising the amino acid sequence of the HCDR2 of monoclonal antibody 2419 (e.g., SEQ ID NO: 18) or a 2419-related antibody; or an HCDR3 comprising the amino acid sequence of the HCDR3 of monoclonal antibody 2419 (e.g., SEQ ID NO: 13) or a 2419-related antibody, and (ii) a VL comprising: an LCDR1 comprising the amino acid sequence of the LCDR1 of monoclonal antibody 2419 (e.g., SEQ ID NO: 14) or a 2419-related antibody; an LCDR2 comprising the amino acid sequence of the LCDR2 of monoclonal antibody 2419 (e.g., SEQ ID NO: 15) or a 2419-related antibody; and an LCDR3 comprising the amino acid sequence of the LCDR3 of monoclonal antibody 2419 (e.g., SEQ ID NO: 16) or a 2419-related antibody.

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 2419 (e.g., SEQ ID NO: 19) or a 2419-related antibody. In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 2419 (e.g., SEQ ID NO: 20) or a 2419-related antibody.

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 2419 (e.g., SEQ ID NO: 19) or a 2419-related antibody; and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 2419 (e.g., SEQ ID NO: 20) or a 2419-related antibody.

In an embodiment, the antibody molecule comprises: (i) a VH comprising the amino acid sequence of the VH of monoclonal antibody 2419 (e.g., SEQ ID NO: 19) or a 2419-related antibody; and (ii) a VL comprising the amino acid sequence of the VL of monoclonal antibody 2419 (e.g., SEQ ID NO: 20) or a 2419-related antibody.

In an embodiment the antibody molecule is monoclonal antibody 2419. In an embodiment, monoclonal antibody 2419 is a humanized monoclonal antibody 2419. In an embodiment, the antibody molecule is a 2419-related antibody molecule, e.g., any of antibodies 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, or 2419-1406, e.g., as disclosed in Table 1 or 5. In an embodiment, the antibody molecule comprises a VH comprising the amino acid sequence of any of SEQ ID NOS: 209-214, 283, 288, 289, 291, 292, 294, 296, or 317, a VL comprising the amino acid sequence of any of SEQ ID NOS: 215-219, 284, 286, 295, or 316, or both.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 2922 (e.g., SEQ ID NO: 21); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 2922 (e.g., SEQ ID NO: 32); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 2922 (e.g., SEQ ID NO: 33).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 2922 (e.g., SEQ ID NO: 34); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 2922 (e.g., SEQ ID NO: 35); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 2922 (e.g., SEQ ID NO: 36).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 2922 (e.g., SEQ ID NO: 21); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 2922 (e.g., SEQ ID NO: 32); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 2922 (e.g., SEQ ID NO: 33), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 2922 (e.g., SEQ ID NO: 34); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 2922 (e.g., SEQ ID NO: 35); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 2922 (e.g., SEQ ID NO: 36).

In an embodiment, the antibody molecule comprises: (i) a VH comprising: an HCDR1 comprising the amino acid sequence of the HCDR1 of monoclonal antibody 2922 (e.g., SEQ ID NO: 21); an HCDR2 comprising the amino acid sequence of the HCDR2 of monoclonal antibody 2922 (e.g., SEQ ID NO: 32); and an HCDR3 comprising the amino acid sequence of the HCDR3 of monoclonal antibody 2922 (e.g., SEQ ID NO: 33), and (ii) a VL comprising: an LCDR1 comprising the amino acid sequence of the LCDR1 of monoclonal antibody 2922 (e.g., SEQ ID NO: 34); an LCDR2 comprising the amino acid sequence of the LCDR2 of monoclonal antibody 2922 (e.g., SEQ ID NO: 35); and an LCDR3 comprising the amino acid sequence of the LCDR3 of monoclonal antibody 2922 (e.g., SEQ ID NO: 36).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 2922 (e.g., SEQ ID NO: 37); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 2922 (e.g., SEQ ID NO: 38); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 2922 (e.g., SEQ ID NO: 33).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 2922 (e.g., SEQ ID NO: 34); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 2922 (e.g., SEQ ID NO: 35); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 2922 (e.g., SEQ ID NO: 36).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 2922 (e.g., SEQ ID NO: 37); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 2922 (e.g., SEQ ID NO: 38); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 2922 (e.g., SEQ ID NO: 33), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 2922 (e.g., SEQ ID NO: 34); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 2922 (e.g., SEQ ID NO: 35); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 2922 (e.g., SEQ ID NO: 36).

In an embodiment, the antibody molecule comprises: (i) a VH comprising: an HCDR1 comprising the amino acid sequence of the HCDR1 of monoclonal antibody 2922 (e.g., SEQ ID NO: 37); an HCDR2 comprising the amino acid sequence of the HCDR2 of monoclonal antibody 2922 (e.g., SEQ ID NO: 38); and an HCDR3 comprising the amino acid sequence of the HCDR3 of monoclonal antibody 2922 (e.g., SEQ ID NO: 33), and (ii) a VL comprising: an LCDR1 comprising the amino acid sequence of the LCDR1 of monoclonal antibody 2922 (e.g., SEQ ID NO: 34); an LCDR2 comprising the amino acid sequence of the LCDR2 of monoclonal antibody 2922 (e.g., SEQ ID NO: 35); and an LCDR3 comprising the amino acid sequence of the LCDR3 of monoclonal antibody 2922 (e.g., SEQ ID NO: 36).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 2922 (e.g., SEQ ID NO: 39). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 2922 (e.g., SEQ ID NO: 40).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 2922 (e.g., SEQ ID NO: 39); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 2922 (e.g., SEQ ID NO: 40). In an embodiment, the antibody molecule comprises: (i) a VH comprising the amino acid sequence of the VH of monoclonal antibody 2922 (e.g., SEQ ID NO: 39); and (ii) a VL comprising the amino acid sequence of the VL of monoclonal antibody 2922 (e.g., SEQ ID NO: 40).

In an embodiment the antibody molecule is monoclonal antibody 2922. In an embodiment, the antibody molecule is a humanized monoclonal antibody 2922.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3327 (e.g., SEQ ID NO: 51); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3327 (e.g., SEQ ID NO: 52); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3327 (e.g., SEQ ID NO: 53).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3327 (e.g., SEQ ID NO: 54); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3327 (e.g., SEQ ID NO: 55); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3327 (e.g., SEQ ID NO: 56).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3327 (e.g., SEQ ID NO: 51); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3327 (e.g., SEQ ID NO: 52); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3327 (e.g., SEQ ID NO: 53), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3327 (e.g., SEQ ID NO: 54); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3327 (e.g., SEQ ID NO: 55); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3327 (e.g., SEQ ID NO: 56).

In an embodiment, the antibody molecule comprises: (i) a VH comprising: an HCDR1 comprising the amino acid sequence of the HCDR1 of monoclonal antibody 3327 (e.g., SEQ ID NO: 51); an HCDR2 comprising the amino acid sequence of the HCDR2 of monoclonal antibody 3327 (e.g., SEQ ID NO: 52); and an HCDR3 comprising the amino acid sequence of the HCDR3 of monoclonal antibody 3327 (e.g., SEQ ID NO: 53), and (ii) a VL comprising: an LCDR1 comprising the amino acid sequence of the LCDR1 of monoclonal antibody 3327 (e.g., SEQ ID NO: 54); an LCDR2 comprising the amino acid sequence of the LCDR2 of monoclonal antibody 3327 (e.g., SEQ ID NO: 55); and an LCDR3 comprising the amino acid sequence of the LCDR3 of monoclonal antibody 3327 (e.g., SEQ ID NO: 56).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3327 (e.g., SEQ ID NO: 57); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3327 (e.g., SEQ ID NO: 58); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3327 (e.g., SEQ ID NO: 53).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3327 (e.g., SEQ ID NO: 54); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3327 (e.g., SEQ ID NO: 55); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3327 (e.g., SEQ ID NO: 56).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3327 (e.g., SEQ ID NO: 57); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3327 (e.g., SEQ ID NO: 58); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3327 (e.g., SEQ ID NO: 53), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3327 (e.g., SEQ ID NO: 54); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3327 (e.g., SEQ ID NO: 55); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3327 (e.g., SEQ ID NO: 56).

In an embodiment, the antibody molecule comprises: (i) a VH comprising: an HCDR1 comprising the amino acid sequence of the HCDR1 of monoclonal antibody 3327 (e.g., SEQ ID NO: 57); an HCDR2 comprising the amino acid sequence of the HCDR2 of monoclonal antibody 3327 (e.g., SEQ ID NO: 58); and an HCDR3 comprising the amino acid sequence of the HCDR3 of monoclonal antibody 3327 (e.g., SEQ ID NO: 53), and (ii) a VL comprising: an LCDR1 comprising the amino acid sequence of the LCDR1 of monoclonal antibody 3327 (e.g., SEQ ID NO: 54); an LCDR2 comprising the amino acid sequence of the LCDR2 of monoclonal antibody 3327 (e.g., SEQ ID NO: 55); and an LCDR3 comprising the amino acid sequence of the LCDR3 of monoclonal antibody 3327 (e.g., SEQ ID NO: 56).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3327 (e.g., SEQ ID NO: 59). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3327 (e.g., SEQ ID NO: 60).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3327 (e.g., SEQ ID NO: 59); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3327 (e.g., SEQ ID NO: 60). In an embodiment, the antibody molecule comprises: (i) a VH comprising the amino acid sequence of the VH of monoclonal antibody 3327 (e.g., SEQ ID NO: 59); and (ii) a VL comprising the amino acid sequence of the VL of monoclonal antibody 3327 (e.g., SEQ ID NO: 60).

In an embodiment the antibody molecule is monoclonal antibody 3327. In an embodiment, the antibody molecule is a humanized monoclonal antibody 3327.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3530 (e.g., SEQ ID NO: 61); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3530 (e.g., SEQ ID NO: 62); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3530 (e.g., SEQ ID NO: 63).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3530 (e.g., SEQ ID NO: 67); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3530 (e.g., SEQ ID NO: 45); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3530 (e.g., SEQ ID NO: 46).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3530 (e.g., SEQ ID NO: 61); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3530 (e.g., SEQ ID NO: 62); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3530 (e.g., SEQ ID NO: 63), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3530 (e.g., SEQ ID NO: 67); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3530 (e.g., SEQ ID NO: 45); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3530 (e.g., SEQ ID NO: 46).

In an embodiment, the antibody molecule comprises: (i) a VH comprising: an HCDR1 comprising the amino acid sequence of the HCDR1 of monoclonal antibody 3530 (e.g., SEQ ID NO: 61); an HCDR2 comprising the amino acid sequence of the HCDR2 of monoclonal antibody 3530 (e.g., SEQ ID NO: 62); and an HCDR3 comprising the amino acid sequence of the HCDR3 of monoclonal antibody 3530 (e.g., SEQ ID NO: 63), and (ii) a VL comprising: an LCDR1 comprising the amino acid sequence of the LCDR1 of monoclonal antibody 3530 (e.g., SEQ ID NO: 67); an LCDR2 comprising the amino acid sequence of the LCDR2 of monoclonal antibody 3530 (e.g., SEQ ID NO: 45); and an LCDR3 comprising the amino acid sequence of the LCDR3 of monoclonal antibody 3530 (e.g., SEQ ID NO: 46).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3530 (e.g., SEQ ID NO: 64); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3530 (e.g., SEQ ID NO: 65); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3530 (e.g., SEQ ID NO: 63).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3530 (e.g., SEQ ID NO: 67); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3530 (e.g., SEQ ID NO: 45); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3530 (e.g., SEQ ID NO: 46).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3530 (e.g., SEQ ID NO: 64); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3530 (e.g., SEQ ID NO: 65); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3530 (e.g., SEQ ID NO: 63), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3530 (e.g., SEQ ID NO: 67); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3530 (e.g., SEQ ID NO: 45); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3530 (e.g., SEQ ID NO: 46).

In an embodiment, the antibody molecule comprises: (i) a VH comprising: an HCDR1 comprising the amino acid sequence of the HCDR1 of monoclonal antibody 3530 (e.g., SEQ ID NO: 64); an HCDR2 comprising the amino acid sequence of the HCDR2 of monoclonal antibody 3530 (e.g., SEQ ID NO: 65); and an HCDR3 comprising the amino acid sequence of the HCDR3 of monoclonal antibody 3530 (e.g., SEQ ID NO: 63), and (ii) a VL comprising: an LCDR1 comprising the amino acid sequence of the LCDR1 of monoclonal antibody 3530 (e.g., SEQ ID NO: 67); an LCDR2 comprising the amino acid sequence of the LCDR2 of monoclonal antibody 3530 (e.g., SEQ ID NO: 45); and an LCDR3 comprising the amino acid sequence of the LCDR3 of monoclonal antibody 3530 (e.g., SEQ ID NO: 46).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3530 (e.g., SEQ ID NO: 66). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3530 (e.g., SEQ ID NO: 70).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3530 (e.g., SEQ ID NO: 66); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3530 (e.g., SEQ ID NO: 70). In an embodiment, the antibody molecule comprises: (i) a VH comprising the amino acid sequence of the VH of monoclonal antibody 3530 (e.g., SEQ ID NO: 66); and (ii) a VL comprising the amino acid sequence of the VL of monoclonal antibody 3530 (e.g., SEQ ID NO: 70).

In an embodiment the antibody molecule is monoclonal antibody 3530. In an embodiment, the antibody molecule is a humanized monoclonal antibody 3530.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3525 (e.g., SEQ ID NO: 61); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3525 (e.g., SEQ ID NO: 62); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3525 (e.g., SEQ ID NO: 63).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3525 (e.g., SEQ ID NO: 44); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3525 (e.g., SEQ ID NO: 45); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3525 (e.g., SEQ ID NO: 46).

In an embodiment, the antibody molecule comprises:
(i) a VH comprising: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3525 (e.g., SEQ ID NO: 61); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3525 (e.g., SEQ ID NO: 62); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3525 (e.g., SEQ ID NO: 63), and
(ii) a VL comprising: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3525 (e.g., SEQ ID NO: 44); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3525 (e.g., SEQ ID NO: 45); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3525 (e.g., SEQ ID NO: 46).

In an embodiment, the antibody molecule comprises: (i) a VH comprising: an HCDR1 comprising the amino acid sequence of the HCDR1 of monoclonal antibody 3525 (e.g., SEQ ID NO: 61); an HCDR2 comprising the amino acid sequence of the HCDR2 of monoclonal antibody 3525 (e.g., SEQ ID NO: 62); and an HCDR3 comprising the amino acid sequence of the HCDR3 of monoclonal antibody 3525 (e.g., SEQ ID NO: 63), and (ii) a VL comprising: an LCDR1 comprising the amino acid sequence of the LCDR1 of monoclonal antibody 3525 (e.g., SEQ ID NO: 44); an LCDR2 comprising the amino acid sequence of the LCDR2 of monoclonal antibody 3525 (e.g., SEQ ID NO: 45); and an LCDR3 comprising the amino acid sequence of the LCDR3 of monoclonal antibody 3525 (e.g., SEQ ID NO: 46).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3525 (e.g., SEQ ID NO: 64); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3525 (e.g., SEQ ID NO: 65); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3525 (e.g., SEQ ID NO: 63).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3525 (e.g., SEQ ID NO: 44); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3525 (e.g., SEQ ID NO: 45); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3525 (e.g., SEQ ID NO: 46).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3525 (e.g., SEQ ID NO: 64); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3525 (e.g., SEQ ID NO: 65); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3525 (e.g., SEQ ID NO: 63), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3525 (e.g., SEQ ID NO: 44); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3525 (e.g., SEQ ID NO: 45); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3525 (e.g., SEQ ID NO: 46).

In an embodiment, the antibody molecule comprises: (i) a VH comprising: an HCDR1 comprising the amino acid sequence of the HCDR1 of monoclonal antibody 3525 (e.g., SEQ ID NO: 64); an HCDR2 comprising the amino acid sequence of the HCDR2 of monoclonal antibody 3525 (e.g., SEQ ID NO: 65); and an HCDR3 comprising the amino acid sequence of the HCDR3 of monoclonal antibody 3525 (e.g., SEQ ID NO: 63), and (ii) a VL comprising: an LCDR1 comprising the amino acid sequence of the LCDR1 of monoclonal antibody 3525 (e.g., SEQ ID NO: 44); an LCDR2 comprising the amino acid sequence of the LCDR2 of monoclonal antibody 3525 (e.g., SEQ ID NO: 45); and an LCDR3 comprising the amino acid sequence of the LCDR3 of monoclonal antibody 3525 (e.g., SEQ ID NO: 46).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3525 (e.g., SEQ ID NO: 66). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3525 (e.g., SEQ ID NO: 50).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3525 (e.g., SEQ ID NO: 66); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3525 (e.g., SEQ ID NO: 50). In an embodiment, the antibody molecule comprises: (i) a VH comprising the amino acid sequence of the VH of monoclonal antibody 3525 (e.g., SEQ ID NO: 66); and (ii) a VL comprising the amino acid sequence of the VL of monoclonal antibody 3525 (e.g., SEQ ID NO: 50).

In an embodiment the antibody molecule is monoclonal antibody 3525. In an embodiment, the antibody molecule is a humanized monoclonal antibody 3525.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 2621 (e.g., SEQ ID NO: 21); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 2621 (e.g., SEQ ID NO: 22); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 2621 (e.g., SEQ ID NO: 23).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 2621 (e.g., SEQ ID NO: 24); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 2621 (e.g., SEQ ID NO: 25); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 2621 (e.g., SEQ ID NO: 26).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 2621 (e.g., SEQ ID NO: 21); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 2621 (e.g., SEQ ID NO: 22); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 2621 (e.g., SEQ ID NO: 23), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 2621 (e.g., SEQ ID NO: 24); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 2621 (e.g., SEQ ID NO: 25); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 2621 (e.g., SEQ ID NO: 26).

In an embodiment, the antibody molecule comprises: (i) a VH comprising: an HCDR1 comprising the amino acid sequence of the HCDR1 of monoclonal antibody 2621 (e.g., SEQ ID NO: 21); an HCDR2 comprising the amino acid sequence of the HCDR2 of monoclonal antibody 2621 (e.g., SEQ ID NO: 22); and an HCDR3 comprising the amino acid sequence of the HCDR3 of monoclonal antibody 2621 (e.g., SEQ ID NO: 23), and (ii) a VL comprising: an LCDR1 comprising the amino acid sequence of the LCDR1 of monoclonal antibody 2621 (e.g., SEQ ID NO: 24); an LCDR2 comprising the amino acid sequence of the LCDR2 of monoclonal antibody 2621 (e.g., SEQ ID NO: 25); and an LCDR3 comprising the amino acid sequence of the LCDR3 of monoclonal antibody 2621 (e.g., SEQ ID NO: 26).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 2621 (e.g., SEQ ID NO: 27); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 2621 (e.g., SEQ ID NO: 28); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 2621 (e.g., SEQ ID NO: 23).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 2621 (e.g., SEQ ID NO: 24); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 2621 (e.g., SEQ ID NO: 25); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 2621 (e.g., SEQ ID NO: 26).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 2621 (e.g., SEQ ID NO: 27); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 2621 (e.g., SEQ ID NO: 28); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 2621 (e.g., SEQ ID NO: 23), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 2621 (e.g., SEQ ID NO: 24); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 2621 (e.g., SEQ ID NO: 25); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 2621 (e.g., SEQ ID NO: 26).

In an embodiment, the antibody molecule comprises: (i) a VH comprising: an HCDR1 comprising the amino acid sequence of the HCDR1 of monoclonal antibody 2621 (e.g., SEQ ID NO: 27); an HCDR2 comprising the amino acid sequence of the HCDR2 of monoclonal antibody 2621 (e.g., SEQ ID NO: 28); and an HCDR3 comprising the amino acid sequence of the HCDR3 of monoclonal antibody 2621 (e.g., SEQ ID NO: 23), and (ii) a VL comprising: an LCDR1 comprising the amino acid sequence of the LCDR1 of monoclonal antibody 2621 (e.g., SEQ ID NO: 24); an LCDR2 comprising the amino acid sequence of the LCDR2 of monoclonal antibody 2621 (e.g., SEQ ID NO: 25); and an LCDR3 comprising the amino acid sequence of the LCDR3 of monoclonal antibody 2621 (e.g., SEQ ID NO: 26).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 2621 (e.g., SEQ ID NO: 29). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 2621 (e.g., SEQ ID NO: 30).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 2621 (e.g., SEQ ID NO: 29); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 2621 (e.g., SEQ ID NO: 30). In an embodiment, the antibody molecule comprises: (i) a VH comprising the amino acid sequence of the VH of monoclonal antibody 2621 (e.g., SEQ ID NO: 29); and (ii) a VL comprising the amino acid sequence of the VL of monoclonal antibody 2621 (e.g., SEQ ID NO: 30).

In an embodiment the antibody molecule is monoclonal antibody 2621. In an embodiment, the antibody molecule is a humanized monoclonal antibody 2621.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3125 (e.g., SEQ ID NO: 11); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3125 (e.g., SEQ ID NO: 42); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3125 (e.g., SEQ ID NO: 43).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3125 (e.g., SEQ ID NO: 44); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3125 (e.g., SEQ ID NO: 45); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3125 (e.g., SEQ ID NO: 46).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3125 (e.g., SEQ ID NO: 11); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3125 (e.g., SEQ ID NO: 42); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3125 (e.g., SEQ ID NO: 43), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3125 (e.g., SEQ ID NO: 44); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3125 (e.g., SEQ ID NO: 45); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3125 (e.g., SEQ ID NO: 46).

In an embodiment, the antibody molecule comprises: (i) a VH comprising: an HCDR1 comprising the amino acid sequence of the HCDR1 of monoclonal antibody 3125 (e.g., SEQ ID NO: 11); an HCDR2 comprising the amino acid sequence of the HCDR2 of monoclonal antibody 3125 (e.g., SEQ ID NO: 42); and an HCDR3 comprising the amino acid sequence of the HCDR3 of monoclonal antibody 3125 (e.g., SEQ ID NO: 43), and (ii) a VL comprising: an LCDR1 comprising the amino acid sequence of the LCDR1 of monoclonal antibody 3125 (e.g., SEQ ID NO: 44); an LCDR2 comprising the amino acid sequence of the LCDR2 of monoclonal antibody 3125 (e.g., SEQ ID NO: 45); and an LCDR3 comprising the amino acid sequence of the LCDR3 of monoclonal antibody 3125 (e.g., SEQ ID NO: 46).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3125 (e.g., SEQ ID NO: 47); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3125 (e.g., SEQ ID NO: 48); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3125 (e.g., SEQ ID NO: 43).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3125 (e.g., SEQ ID NO: 44); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3125 (e.g., SEQ ID NO: 45); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3125 (e.g., SEQ ID NO: 46).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3125 (e.g., SEQ ID NO: 47); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3125 (e.g., SEQ ID NO: 48); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3125 (e.g., SEQ ID NO: 43), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3125 (e.g., SEQ ID NO: 44); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3125 (e.g., SEQ ID NO:45); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3125 (e.g., SEQ ID NO: 46).

In an embodiment, the antibody molecule comprises: (i) a VH comprising: an HCDR1 comprising the amino acid sequence of the HCDR1 of monoclonal antibody 3125 (e.g., SEQ ID NO: 47); an HCDR2 comprising the amino acid sequence of the HCDR2 of monoclonal antibody 3125 (e.g., SEQ ID NO: 48); and an HCDR3 comprising the amino acid sequence of the HCDR3 of monoclonal antibody 3125 (e.g., SEQ ID NO: 43), and (ii) a VL comprising: an LCDR1 comprising the amino acid sequence of the LCDR1 of monoclonal antibody 3125 (e.g., SEQ ID NO: 44); an LCDR2 comprising the amino acid sequence of the LCDR2 of monoclonal antibody 3125 (e.g., SEQ ID NO: 45); and an LCDR3 comprising the amino acid sequence of the LCDR3 of monoclonal antibody 3125 (e.g., SEQ ID NO: 46).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3125 (e.g., SEQ ID NO: 49). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3125 (e.g., SEQ ID NO: 50).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3125 (e.g., SEQ ID NO: 49); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3125 (e.g., SEQ ID NO: 50). In an embodiment, the antibody molecule comprises: (i) a VH comprising the amino acid sequence of the VH of monoclonal antibody 3125 (e.g., SEQ ID NO: 49); and (ii) a VL comprising the amino acid sequence of the VL of monoclonal antibody 3125 (e.g., SEQ ID NO: 50).

In an embodiment the antibody molecule is monoclonal antibody 3125. In an embodiment, the antibody molecule is a humanized monoclonal antibody 3125.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4035 or 4035-062 (e.g., SEQ ID NO: 93); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4035 or 4035-062 (e.g., SEQ ID NO: 94); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4035 or 4035-062 (e.g., SEQ ID NO: 95).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4035 or 4035-062 (e.g., SEQ ID NO: 96); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4035 or 4035-062 (e.g., SEQ ID NO: 97); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4035 or 4035-062 (e.g., SEQ ID NO: 98).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4035 or 4035-062 (e.g., SEQ ID NO: 93); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4035 or 4035-062 (e.g., SEQ ID NO: 94); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4035 or 4035-062 (e.g., SEQ ID NO: 95), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4035 or 4035-062 (e.g., SEQ ID NO: 96); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4035 or 4035-062 (e.g., SEQ ID NO: 97); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4035 or 4035-062 (e.g., SEQ ID NO: 98).

In an embodiment, the antibody molecule comprises: (i) a VH comprising: an HCDR1 comprising the amino acid sequence of the HCDR1 of monoclonal antibody 4035 or 4035-062 (e.g., SEQ ID NO: 93); an HCDR2 comprising the amino acid sequence of the HCDR2 of monoclonal antibody 4035 or 4035-062 (e.g., SEQ ID NO: 94); and an HCDR3 comprising the amino acid sequence of the HCDR3 of monoclonal antibody 4035 or 4035-062 (e.g., SEQ ID NO: 95), and (ii) a VL comprising: an LCDR1 comprising the amino acid sequence of the LCDR1 of monoclonal antibody 4035 or 4035-062 (e.g., SEQ ID NO: 96); an LCDR2 comprising the amino acid sequence of the LCDR2 of monoclonal antibody 4035 or 4035-062 (e.g., SEQ ID NO: 97); and an LCDR3 comprising the amino acid sequence of the LCDR3 of monoclonal antibody 4035 or 4035-062 (e.g., SEQ ID NO: 98).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4035 or 4035-062 (e.g., SEQ ID NO: 99); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4035 (e.g., SEQ ID NO: 100) or 4035-062 (e.g., SEQ ID NO: 273); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4035 or 4035-062 (e.g., SEQ ID NO: 95).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4035 or 4035-062 (e.g., SEQ ID NO: 96); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4035 or 4035-062 (e.g., SEQ ID NO: 97); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4035 or 4035-062 (e.g., SEQ ID NO: 98).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4035 or 4035-062 (e.g., SEQ ID NO: 99); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4035 (e.g., SEQ ID NO: 100) or 4035-062 (e.g., SEQ ID NO: 273); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4035 or 4035-062 (e.g., SEQ ID NO: 95), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4035 or 4035-062 (e.g., SEQ ID NO: 96); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4035 or 4035-062 (e.g., SEQ ID NO: 97); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4035 or 4035-062 (e.g., SEQ ID NO: 98).

In an embodiment, the antibody molecule comprises: (i) a VH comprising: an HCDR1 comprising the amino acid sequence of the HCDR1 of monoclonal antibody 4035 or 4035-062 (e.g., SEQ ID NO: 99); an HCDR2 comprising the amino acid sequence of the HCDR2 of monoclonal antibody 4035 (e.g., SEQ ID NO: 100) or 4035-062 (e.g., SEQ ID NO: 273); and an HCDR3 comprising the amino acid sequence of the HCDR3 of monoclonal antibody 4035 or 4035-062 (e.g., SEQ ID NO: 95), and (ii) a VL comprising: an LCDR1 comprising the amino acid sequence of the LCDR1 of monoclonal antibody 4035 or 4035-062 (e.g., SEQ ID NO: 96); an LCDR2 comprising the amino acid sequence of the LCDR2 of monoclonal antibody 4035 or 4035-062 (e.g., SEQ ID NO: 97); and an LCDR3 comprising the amino acid sequence of the LCDR3 of monoclonal antibody 4035 or 4035-062 (e.g., SEQ ID NO: 98).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 4035 (e.g., SEQ ID NO: 101) or 4035-062 (e.g., SEQ ID NO: 225). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 4035 (e.g., SEQ ID NO: 102) or 4035-062 (e.g., SEQ ID NO: 229).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 4035 (e.g., SEQ ID NO: 101) or 4035-062 (e.g., SEQ ID NO: 225); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 4035 (e.g., SEQ ID NO: 102) or 4035-062 (e.g., SEQ ID NO: 229). In an embodiment, the antibody molecule comprises: (i) a VH comprising the amino acid sequence of the VH of monoclonal antibody 4035 (e.g., SEQ ID NO: 101) or 4035-062 (e.g., SEQ ID NO: 225); and (ii) a VL comprising the amino acid sequence of the VL of monoclonal antibody 4035 (e.g., SEQ ID NO: 102) or 4035-062 (e.g., SEQ ID NO: 229).

In an embodiment, the antibody molecule is monoclonal antibody 4035. In an embodiment, monoclonal antibody 4035 is a humanized monoclonal antibody 4035 (e.g., antibody 4035-062). In another embodiment, the antibody molecule is antibody 4035-062. In an embodiment, the antibody molecule comprises a VH comprising the amino acid sequence of any of SEQ ID NOS: 220-227 or 262-265, a VL comprising the amino acid sequence of any of SEQ ID NOS: 228-234, or both.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3934 (e.g., SEQ ID NO: 103); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3934 (e.g., SEQ ID NO: 104); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3934 (e.g., SEQ ID NO: 105).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3934 (e.g., SEQ ID NO: 106); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3934 (e.g., SEQ ID NO: 107); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3934 (e.g., SEQ ID NO: 108).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3934 (e.g., SEQ ID NO: 103); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3934 (e.g., SEQ ID NO: 104); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3934 (e.g., SEQ ID NO: 105), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3934 (e.g., SEQ ID NO: 106); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3934 (e.g., SEQ ID NO: 107); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3934 (e.g., SEQ ID NO: 108).

In an embodiment, the antibody molecule comprises: (i) a VH comprising: an HCDR1 comprising the amino acid sequence of the HCDR1 of monoclonal antibody 3934 (e.g., SEQ ID NO: 103); an HCDR2 comprising the amino acid sequence of the HCDR2 of monoclonal antibody 3934 (e.g., SEQ ID NO: 104); and an HCDR3 comprising the amino acid sequence of the HCDR3 of monoclonal antibody 3934 (e.g., SEQ ID NO: 105), and (ii) a VL comprising: an LCDR1 comprising the amino acid sequence of the LCDR1 of monoclonal antibody 3934 (e.g., SEQ ID NO: 106); an LCDR2 comprising the amino acid sequence of the LCDR2 of monoclonal antibody 3934 (e.g., SEQ ID NO: 107); and an LCDR3 comprising the amino acid sequence of the LCDR3 of monoclonal antibody 3934 (e.g., SEQ ID NO: 108).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3934 (e.g., SEQ ID NO: 109); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3934 (e.g., SEQ ID NO: 110); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3934 (e.g., SEQ ID NO: 105).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3934 (e.g., SEQ ID NO: 106); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3934 (e.g., SEQ ID NO: 107); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3934 (e.g., SEQ ID NO: 108).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3934 (e.g., SEQ ID NO: 109); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3934 (e.g., SEQ ID NO: 110); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3934 (e.g., SEQ ID NO: 105), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3934 (e.g., SEQ ID NO: 106); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3934 (e.g., SEQ ID NO: 107); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3934 (e.g., SEQ ID NO: 108).

In an embodiment, the antibody molecule comprises: (i) a VH comprising: an HCDR1 comprising the amino acid sequence of the HCDR1 of monoclonal antibody 3934 (e.g., SEQ ID NO: 109); an HCDR2 comprising the amino acid sequence of the HCDR2 of monoclonal antibody 3934 (e.g., SEQ ID NO: 110); and an HCDR3 comprising the amino acid sequence of the HCDR3 of monoclonal antibody 3934 (e.g., SEQ ID NO: 105), and (ii) a VL comprising: an LCDR1 comprising the amino acid sequence of the LCDR1 of monoclonal antibody 3934 (e.g., SEQ ID NO: 106); an LCDR2 comprising the amino acid sequence of the LCDR2 of monoclonal antibody 3934 (e.g., SEQ ID NO: 107); and an LCDR3 comprising the amino acid sequence of the LCDR3 of monoclonal antibody 3934 (e.g., SEQ ID NO: 108).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3934 (e.g., SEQ ID NO: 111). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3934 (e.g., SEQ ID NO: 112).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3934 (e.g., SEQ ID NO: 111); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3934 (e.g., SEQ ID NO: 112). In an embodiment, the antibody molecule comprises: (i) a VH comprising the amino acid sequence of the VH of monoclonal antibody 3934 (e.g., SEQ ID NO: 111); and (ii) a VL comprising the amino acid sequence of the VL of monoclonal antibody 3934 (e.g., SEQ ID NO: 112).

In an embodiment the antibody molecule is monoclonal antibody 3934. In an embodiment, the antibody molecule is a humanized monoclonal antibody 3934.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3833 (e.g., SEQ ID NO: 112); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3833 (e.g., SEQ ID NO: 113); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3833 (e.g., SEQ ID NO: 114).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3833 (e.g., SEQ ID NO: 115); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3833 (e.g., SEQ ID NO: 116); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3833 (e.g., SEQ ID NO: 117).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3833 (e.g., SEQ ID NO: 113); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3833 (e.g., SEQ ID NO: 114); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3833 (e.g., SEQ ID NO: 115), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3833 (e.g., SEQ ID NO: 116); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3833 (e.g., SEQ ID NO: 117); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3833 (e.g., SEQ ID NO: 118).

In an embodiment, the antibody molecule comprises: (i) a VH comprising: an HCDR1 comprising the amino acid sequence of the HCDR1 of monoclonal antibody 3833 (e.g., SEQ ID NO: 113); an HCDR2 comprising the amino acid sequence of the HCDR2 of monoclonal antibody 3833 (e.g., SEQ ID NO: 114); and an HCDR3 comprising the amino acid sequence of the HCDR3 of monoclonal antibody 3833 (e.g., SEQ ID NO: 115), and (ii) a VL comprising: an LCDR1 comprising the amino acid sequence of the LCDR1 of monoclonal antibody 3833 (e.g., SEQ ID NO: 116); an LCDR2 comprising the amino acid sequence of the LCDR2 of monoclonal antibody 3833 (e.g., SEQ ID NO: 117); and an LCDR3 comprising the amino acid sequence of the LCDR3 of monoclonal antibody 3833 (e.g., SEQ ID NO: 118).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3833 (e.g., SEQ ID NO: 119); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3833 (e.g., SEQ ID NO: 120); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3833 (e.g., SEQ ID NO: 115).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3833 (e.g., SEQ ID NO: 116); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3833 (e.g., SEQ ID NO: 117); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3833 (e.g., SEQ ID NO: 118).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3833 (e.g., SEQ ID NO: 119); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3833 (e.g., SEQ ID NO: 120); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3833 (e.g., SEQ ID NO: 115), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3833 (e.g., SEQ ID NO: 116); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3833 (e.g., SEQ ID NO: 117); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3833 (e.g., SEQ ID NO: 118).

In an embodiment, the antibody molecule comprises: (i) a VH comprising: an HCDR1 comprising the amino acid sequence of the HCDR1 of monoclonal antibody 3833 (e.g., SEQ ID NO: 119); an HCDR2 comprising the amino acid sequence of the HCDR2 of monoclonal antibody 3833 (e.g., SEQ ID NO: 120); and an HCDR3 comprising the amino acid sequence of the HCDR3 of monoclonal antibody 3833 (e.g., SEQ ID NO: 115), and (ii) a VL comprising: an LCDR1 comprising the amino acid sequence of the LCDR1 of monoclonal antibody 3833 (e.g., SEQ ID NO: 116); an LCDR2 comprising the amino acid sequence of the LCDR2 of monoclonal antibody 3833 (e.g., SEQ ID NO: 117); and an LCDR3 comprising the amino acid sequence of the LCDR3 of monoclonal antibody 3833 (e.g., SEQ ID NO: 118).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3833 (e.g., SEQ ID NO: 121). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3833 (e.g., SEQ ID NO: 122).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3833 (e.g., SEQ ID NO: 121); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3833 (e.g., SEQ ID NO: 122). In an embodiment, the antibody molecule comprises: (i) a VH comprising the amino acid sequence of the VH of monoclonal antibody 3833 (e.g., SEQ ID NO: 121); and (ii) a VL comprising the amino acid sequence of the VL of monoclonal antibody 3833 (e.g., SEQ ID NO: 122).

In an embodiment the antibody molecule is monoclonal antibody 3833. In an embodiment, monoclonal antibody 3833 is a humanized monoclonal antibody 3833. In an embodiment, the antibody molecule comprises a VH comprising the amino acid sequence of any of SEQ ID NO: 246-250, a VL comprising the amino acid sequence of any of SEQ ID NO: 251-253, or both.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3631 (e.g., SEQ ID NO: 123); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3631 (e.g., SEQ ID NO: 124); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3631 (e.g., SEQ ID NO: 125).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3631 (e.g., SEQ ID NO: 126); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3631 (e.g., SEQ ID NO: 127); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3631 (e.g., SEQ ID NO: 128).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3631 (e.g., SEQ ID NO: 123); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3631 (e.g., SEQ ID NO: 124); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3631 (e.g., SEQ ID NO: 125), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3631 (e.g., SEQ ID NO: 126); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3631 (e.g., SEQ ID NO: 127); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3631 (e.g., SEQ ID NO: 128).

In an embodiment, the antibody molecule comprises: (i) a VH comprising: an HCDR1 comprising the amino acid sequence of the HCDR1 of monoclonal antibody 3631 (e.g., SEQ ID NO: 123); an HCDR2 comprising the amino acid sequence of the HCDR2 of monoclonal antibody 3631 (e.g., SEQ ID NO: 124); and an HCDR3 comprising the amino acid sequence of the HCDR3 of monoclonal antibody 3631 (e.g., SEQ ID NO: 125), and (ii) a VL comprising: an LCDR1 comprising the amino acid sequence of the LCDR1 of monoclonal antibody 3631 (e.g., SEQ ID NO: 126); an LCDR2 comprising the amino acid sequence of the LCDR2 of monoclonal antibody 3631 (e.g., SEQ ID NO: 127); and an LCDR3 comprising the amino acid sequence of the LCDR3 of monoclonal antibody 3631 (e.g., SEQ ID NO: 128).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3631 (e.g., SEQ ID NO: 129); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3631 (e.g., SEQ ID NO: 130); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3631 (e.g., SEQ ID NO: 125).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3631 (e.g., SEQ ID NO: 126); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3631 (e.g., SEQ ID NO: 127); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3631 (e.g., SEQ ID NO: 128).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3631 (e.g., SEQ ID NO: 129); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3631 (e.g., SEQ ID NO: 130); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3631 (e.g., SEQ ID NO: 125), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3631 (e.g., SEQ ID NO: 126); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3631 (e.g., SEQ ID NO:45); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3631 (e.g., SEQ ID NO: 128).

In an embodiment, the antibody molecule comprises: (i) a VH comprising: an HCDR1 comprising the amino acid sequence of the HCDR1 of monoclonal antibody 3631 (e.g., SEQ ID NO: 129); an HCDR2 comprising the amino acid sequence of the HCDR2 of monoclonal antibody 3631 (e.g., SEQ ID NO: 130); and an HCDR3 comprising the amino acid sequence of the HCDR3 of monoclonal antibody 3631 (e.g., SEQ ID NO: 125), and (ii) a VL comprising: an LCDR1 comprising the amino acid sequence of the LCDR1 of monoclonal antibody 3631 (e.g., SEQ ID NO: 126); an LCDR2 comprising the amino acid sequence of the LCDR2 of monoclonal antibody 3631 (e.g., SEQ ID NO: 127); and an LCDR3 comprising the amino acid sequence of the LCDR3 of monoclonal antibody 3631 (e.g., SEQ ID NO: 128).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3631 (e.g., SEQ ID NO: 131). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3631 (e.g., SEQ ID NO: 132).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3631 (e.g., SEQ ID NO: 131); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3631 (e.g., SEQ ID NO: 132). In an embodiment, the antibody molecule comprises: (i) a VH comprising the amino acid sequence of the VH of monoclonal antibody 3631 (e.g., SEQ ID NO: 131); and (ii) a VL comprising the amino acid sequence of the VL of monoclonal antibody 3631 (e.g., SEQ ID NO: 132).

In an embodiment the antibody molecule is monoclonal antibody 3631. In an embodiment, the antibody molecule is a humanized monoclonal antibody 3631.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3732 (e.g., SEQ ID NO: 133); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3732 (e.g., SEQ ID NO: 134); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3732 (e.g., SEQ ID NO: 135).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3732 (e.g., SEQ ID NO: 136); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3732 (e.g., SEQ ID NO: 127); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3732 (e.g., SEQ ID NO: 137).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3732 (e.g., SEQ ID NO: 133); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3732 (e.g., SEQ ID NO: 134); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3732 (e.g., SEQ ID NO: 135), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3732 (e.g., SEQ ID NO: 136); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3732 (e.g., SEQ ID NO: 127); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3732 (e.g., SEQ ID NO: 137).

In an embodiment, the antibody molecule comprises: (i) a VH comprising: an HCDR1 comprising the amino acid sequence of the HCDR1 of monoclonal antibody 3732 (e.g., SEQ ID NO: 133); an HCDR2 comprising the amino acid sequence of the HCDR2 of monoclonal antibody 3732 (e.g., SEQ ID NO: 134); and an HCDR3 comprising the amino acid sequence of the HCDR3 of monoclonal antibody 3732 (e.g., SEQ ID NO: 135), and (ii) a VL comprising: an LCDR1 comprising the amino acid sequence of the LCDR1 of monoclonal antibody 3732 (e.g., SEQ ID NO: 136); an LCDR2 comprising the amino acid sequence of the LCDR2 of monoclonal antibody 3732 (e.g., SEQ ID NO: 127); and an LCDR3 comprising the amino acid sequence of the LCDR3 of monoclonal antibody 3732 (e.g., SEQ ID NO: 137).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3732 (e.g., SEQ ID NO: 138); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3732 (e.g., SEQ ID NO: 139); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3732 (e.g., SEQ ID NO: 135).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3732 (e.g., SEQ ID NO: 136); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3732 (e.g., SEQ ID NO: 127); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3732 (e.g., SEQ ID NO: 137).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3732 (e.g., SEQ ID NO: 138); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3732 (e.g., SEQ ID NO: 139); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3732 (e.g., SEQ ID NO: 135), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3732 (e.g., SEQ ID NO: 136); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3732 (e.g., SEQ ID NO: 127); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3732 (e.g., SEQ ID NO: 137).

In an embodiment, the antibody molecule comprises: (i) a VH comprising: an HCDR1 comprising the amino acid sequence of the HCDR1 of monoclonal antibody 3732 (e.g., SEQ ID NO: 138); an HCDR2 comprising the amino acid sequence of the HCDR2 of monoclonal antibody 3732 (e.g., SEQ ID NO: 139); and an HCDR3 comprising the amino acid sequence of the HCDR3 of monoclonal antibody 3732 (e.g., SEQ ID NO: 135), and (ii) a VL comprising: an LCDR1 comprising the amino acid sequence of the LCDR1 of monoclonal antibody 3732 (e.g., SEQ ID NO: 136); an LCDR2 comprising the amino acid sequence of the LCDR2 of monoclonal antibody 3732 (e.g., SEQ ID NO: 127); and an LCDR3 comprising the amino acid sequence of the LCDR3 of monoclonal antibody 3732 (e.g., SEQ ID NO: 137).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3732 (e.g., SEQ ID NO: 140). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3732 (e.g., SEQ ID NO: 141).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3732 (e.g., SEQ ID NO: 140); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3732 (e.g., SEQ ID NO: 141). In an embodiment, the antibody molecule comprises: (i) a VH comprising the amino acid sequence of the VH of monoclonal antibody 3732 (e.g., SEQ ID NO: 140); and (ii) a VL comprising the amino acid sequence of the VL of monoclonal antibody 3732 (e.g., SEQ ID NO: 141).

In an embodiment the antibody molecule is monoclonal antibody 3732. In an embodiment, the antibody molecule is a humanized monoclonal antibody 3732.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4338 (e.g., SEQ ID NO: 11); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4338 (e.g., SEQ ID NO: 142); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4338 (e.g., SEQ ID NO: 143).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4338 (e.g., SEQ ID NO: 144 or 146); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4338 (e.g., SEQ ID NO: 107 or 147); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4338 (e.g., SEQ ID NO: 145 or 148).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4338 (e.g., SEQ ID NO: 11); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4338 (e.g., SEQ ID NO: 142); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4338 (e.g., SEQ ID NO: 143), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4338 (e.g., SEQ ID NO: 144 or 146); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4338 (e.g., SEQ ID NO: 107 or 147); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4338 (e.g., SEQ ID NO: 145 or 148).

In an embodiment, the antibody molecule comprises: (i) a VH comprising: an HCDR1 comprising the amino acid sequence of the HCDR1 of monoclonal antibody 4338 (e.g., SEQ ID NO: 11); an HCDR2 comprising the amino acid sequence of the HCDR2 of monoclonal antibody 4338 (e.g., SEQ ID NO: 142); and an HCDR3 comprising the amino acid sequence of the HCDR3 of monoclonal antibody 4338 (e.g., SEQ ID NO: 143), and (ii) a VL comprising: an LCDR1 comprising the amino acid sequence of the LCDR1 of monoclonal antibody 4338 (e.g., SEQ ID NO: 144 or 146); an LCDR2 comprising the amino acid sequence of the LCDR2 of monoclonal antibody 4338 (e.g., SEQ ID NO: 107 or 147); and an LCDR3 comprising the amino acid sequence of the LCDR3 of monoclonal antibody 4338 (e.g., SEQ ID NO: 145 or 148).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4338 (e.g., SEQ ID NO: 149); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4338 (e.g., SEQ ID NO: 150); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4338 (e.g., SEQ ID NO: 143).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4338 (e.g., SEQ ID NO: 144 or 146); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4338 (e.g., SEQ ID NO: 107 or 147); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4338 (e.g., SEQ ID NO: 145 or 148).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4338 (e.g., SEQ ID NO: 149); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4338 (e.g., SEQ ID NO: 150); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4338 (e.g., SEQ ID NO: 143), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4338 (e.g., SEQ ID NO: 144 or 146); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4338 (e.g., SEQ ID NO: 107 or 147); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4338 (e.g., SEQ ID NO: 145 or 148).

In an embodiment, the antibody molecule comprises: (i) a VH comprising: an HCDR1 comprising the amino acid sequence of the HCDR1 of monoclonal antibody 4338 (e.g., SEQ ID NO: 149); an HCDR2 comprising the amino acid sequence of the HCDR2 of monoclonal antibody 4338 (e.g., SEQ ID NO: 150); and an HCDR3 comprising the amino acid sequence of the HCDR3 of monoclonal antibody 4338 (e.g., SEQ ID NO: 143), and (ii) a VL comprising: an LCDR1 comprising the amino acid sequence of the LCDR1 of monoclonal antibody 4338 (e.g., SEQ ID NO: 144 or 146); an LCDR2 comprising the amino acid sequence of the LCDR2 of monoclonal antibody 4338 (e.g., SEQ ID NO: 107 or 147); and an LCDR3 comprising the amino acid sequence of the LCDR3 of monoclonal antibody 4338 (e.g., SEQ ID NO: 145 or 148).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 4338 (e.g., SEQ ID NO: 151). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 4338 (e.g., SEQ ID NO: 152 or 153).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 4338 (e.g., SEQ ID NO: 151); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 4338 (e.g., SEQ ID NO: 152 or 153). In an embodiment, the antibody molecule comprises: (i) a VH comprising the amino acid sequence of the VH of monoclonal antibody 4338 (e.g., SEQ ID NO: 150); and (ii) a VL comprising the amino acid sequence of the VL of monoclonal antibody 4338 (e.g., SEQ ID NO: 152 or 153).

In an embodiment the antibody molecule is monoclonal antibody 4338. In an embodiment, the antibody molecule is a humanized monoclonal antibody 4338.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4540, 4540-063, or 4540-033 (e.g., SEQ ID NO: 154); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4540, 4540-063, or 4540-033 (e.g., SEQ ID NO: 155); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4540, 4540-063, or 4540-033 (e.g., SEQ ID NO: 156).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4540 (e.g., SEQ ID NO: 116), 4540-063 (e.g., SEQ ID NO: 274), or 4540-033 (e.g., SEQ ID NO: 274); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4540 (e.g., SEQ ID NO: 157), 4540-063 (e.g., SEQ ID NO: 275), or 4540-033 (e.g., SEQ ID NO: 275); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4540, 4540-063, or 4540-033 (e.g., SEQ ID NO: 158).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4540, 4540-063, or 4540-033 (e.g., SEQ ID NO: 154); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4540, 4540-063, or 4540-033 (e.g., SEQ ID NO: 155); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4540, 4540-063, or 4540-033 (e.g., SEQ ID NO: 156), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4540 (e.g., SEQ ID NO: 116), 4540-063 (e.g., SEQ ID NO: 274), or 4540-033 (e.g., SEQ ID NO: 274); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4540 (e.g., SEQ ID NO: 157), 4540-063 (e.g., SEQ ID NO: 275), or 4540-033 (e.g., SEQ ID NO: 275); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4540, 4540-063, or 4540-033 (e.g., SEQ ID NO: 158).

In an embodiment, the antibody molecule comprises: (i) a VH comprising three heavy chain complementarity determining regions (HCDR1, HCDR2, and HCDR3), wherein the heavy chain variable region comprises: an HCDR1 comprises the amino acid sequence of the HCDR1 of monoclonal antibody 4540, 4540-063, or 4540-033 (e.g., SEQ ID NO: 154); an HCDR2 comprising the amino acid sequence of the HCDR2 of monoclonal antibody 4540, 4540-063, or 4540-033 (e.g., SEQ ID NO: 155); and an HCDR3 comprising the amino acid sequence of the HCDR3 of monoclonal antibody 4540, 4540-063, or 4540-033 (e.g., SEQ ID NO: 156), and (ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of monoclonal antibody 4540 (e.g., SEQ ID NO: 116), 4540-063 (e.g., SEQ ID NO: 274), or 4540-033 (e.g., SEQ ID NO: 274); an LCDR2 comprising the amino acid sequence of the LCDR2 of monoclonal antibody 4540 (e.g., SEQ ID NO: 157), 4540-063 (e.g., SEQ ID NO: 275), or 4540-033 (e.g., SEQ ID NO: 275); and an LCDR3 comprising the amino acid sequence of the LCDR3 of monoclonal antibody 4540, 4540-063, or 4540-033 (e.g., SEQ ID NO: 158).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4540 (e.g., SEQ ID NO: 159), 4540-063 (e.g., SEQ ID NO: 276), or 4540-033 (e.g., SEQ ID NO: 159); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4540 (e.g., SEQ ID NO: 160), 4540-063 (e.g., SEQ ID NO: 277), or 4540-033 (e.g., SEQ ID NO: 278); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4540, 4540-063, or 4540-033 (e.g., SEQ ID NO: 156).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4540 (e.g., SEQ ID NO: 116), 4540-063 (e.g., SEQ ID NO: 274), or 4540-033 (e.g., SEQ ID NO: 274); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4540 (e.g., SEQ ID NO: 157), 4540-063 (e.g., SEQ ID NO: 275), or 4540-033 (e.g., SEQ ID NO: 275); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4540, 4540-063, or 4540-033 (e.g., SEQ ID NO: 158).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4540 (e.g., SEQ ID NO: 159), 4540-063 (e.g., SEQ ID NO: 276), or 4540-033 (e.g., SEQ ID NO: 159); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4540 (e.g., SEQ ID NO: 160), 4540-063 (e.g., SEQ ID NO: 277), or 4540-033 (e.g., SEQ ID NO: 278); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4540, 4540-063, or 4540-033 (e.g., SEQ ID NO: 156), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4540 (e.g., SEQ ID NO: 116), 4540-063 (e.g., SEQ ID NO: 274), or 4540-033 (e.g., SEQ ID NO: 274); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4540 (e.g., SEQ ID NO: 157), 4540-063 (e.g., SEQ ID NO: 275), or 4540-033 (e.g., SEQ ID NO: 275); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4540, 4540-063, or 4540-033 (e.g., SEQ ID NO: 158).

In an embodiment, the antibody molecule comprises: (i) a VH comprising one, two, or all of the following: an HCDR1 comprising the amino acid sequence of the HCDR1 of monoclonal antibody 4540 (e.g., SEQ ID NO: 159), 4540-063 (e.g., SEQ ID NO: 276), or 4540-033 (e.g., SEQ ID NO: 159); an HCDR2 comprising the amino acid sequence of the HCDR2 of monoclonal antibody 4540 (e.g., SEQ ID NO: 160), 4540-063 (e.g., SEQ ID NO: 277), or 4540-033 (e.g., SEQ ID NO: 278); or an HCDR3 comprising the amino acid sequence of the HCDR3 of monoclonal antibody 4540, 4540-063, or 4540-033 (e.g., SEQ ID NO: 156), and (ii) a VL comprising one, two, or all of the following: an LCDR1 comprising the amino acid sequence of the LCDR1 of monoclonal antibody 4540 (e.g., SEQ ID NO: 116), 4540-063 (e.g., SEQ ID NO: 274), or 4540-033 (e.g., SEQ ID NO: 274); an LCDR2 comprising the amino acid sequence of the LCDR2 of monoclonal antibody 4540 (e.g., SEQ ID NO: 157), 4540-063 (e.g., SEQ ID NO: 275), or 4540-033 (e.g., SEQ ID NO: 275); or an LCDR3 comprising the amino acid sequence of the LCDR3 of monoclonal antibody 4540, 4540-063, or 4540-033 (e.g., SEQ ID NO: 158).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 4540 (e.g., SEQ ID NO: 161), 4540-063 (e.g., SEQ ID NO: 258), or 4540-033 (e.g., SEQ ID NO: 256). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 4540 (e.g., SEQ ID NO: 162), 4540-063 (e.g., SEQ ID NO: 261), or 4540-033 (e.g., SEQ ID NO: 261).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 4540 (e.g., SEQ ID NO: 161), 4540-063 (e.g., SEQ ID NO: 258), or 4540-033 (e.g., SEQ ID NO: 256); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 4540 (e.g., SEQ ID NO: 162), 4540-063 (e.g., SEQ ID NO: 261), or 4540-033 (e.g., SEQ ID NO: 261). In an embodiment, the antibody molecule comprises: (i) a VH comprising the amino acid sequence of the VH of monoclonal antibody 4540 (e.g., SEQ ID NO: 161), 4540-063 (e.g., SEQ ID NO: 258), or 4540-033 (e.g., SEQ ID NO: 256); and (ii) a VL comprising the amino acid sequence of the VL of monoclonal antibody 4540 (e.g., SEQ ID NO: 162), 4540-063 (e.g., SEQ ID NO: 261), or 4540-033 (e.g., SEQ ID NO: 261).

In an embodiment the antibody molecule is monoclonal antibody 4540, 4540-063, or 4540-033.

In an embodiment, monoclonal antibody 4540 is a humanized monoclonal antibody 4540 (e.g., antibodies 4540-063 or 4540-033). In an embodiment, the antibody molecule comprises a VH comprising the amino acid sequence of any of SEQ ID NOS: 254-258, a VL comprising the amino acid sequence of any of SEQ ID NOS: 259-261, or both.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4237 (e.g., SEQ ID NO: 163); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4237 (e.g., SEQ ID NO: 164); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4237 (e.g., SEQ ID NO: 165).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4237 (e.g., SEQ ID NO: 166); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4237 (e.g., SEQ ID NO: 167); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4237 (e.g., SEQ ID NO: 168).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4237 (e.g., SEQ ID NO: 163); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4237 (e.g., SEQ ID NO: 164); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4237 (e.g., SEQ ID NO: 165), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4237 (e.g., SEQ ID NO: 166); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4237 (e.g., SEQ ID NO: 167); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4237 (e.g., SEQ ID NO: 168).

In an embodiment, the antibody molecule comprises: (i) a VH comprising: an HCDR1 comprising the amino acid sequence of the HCDR1 of monoclonal antibody 4237 (e.g., SEQ ID NO: 163); an HCDR2 comprising the amino acid sequence of the HCDR2 of monoclonal antibody 4237 (e.g., SEQ ID NO: 164); and an HCDR3 comprising the amino acid sequence of the HCDR3 of monoclonal antibody 4237 (e.g., SEQ ID NO: 165), and (ii) a VL comprising: an LCDR1 comprising the amino acid sequence of the LCDR1 of monoclonal antibody 4237 (e.g., SEQ ID NO: 166); an LCDR2 comprising the amino acid sequence of the LCDR2 of monoclonal antibody 4237 (e.g., SEQ ID NO: 167); and an LCDR3 comprising the amino acid sequence of the LCDR3 of monoclonal antibody 4237 (e.g., SEQ ID NO: 168).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4237 (e.g., SEQ ID NO: 169); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4237 (e.g., SEQ ID NO: 170); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4237 (e.g., SEQ ID NO: 165).

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4237 (e.g., SEQ ID NO: 166); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4237 (e.g., SEQ ID NO: 167); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4237 (e.g., SEQ ID NO: 168).

In an embodiment, the antibody molecule comprises:
(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4237 (e.g., SEQ ID NO: 169); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4237 (e.g., SEQ ID NO: 170); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4237 (e.g., SEQ ID NO: 165), and
(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4237 (e.g., SEQ ID NO: 166); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4237 (e.g., SEQ ID NO: 167); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4237 (e.g., SEQ ID NO: 168).

In an embodiment, the antibody molecule comprises: (i) a VH comprising: an HCDR1 comprising the amino acid sequence of the HCDR1 of monoclonal antibody 4237 (e.g., SEQ ID NO: 169); an HCDR2 comprising the amino acid sequence of the HCDR2 of monoclonal antibody 4237 (e.g., SEQ ID NO: 170); and an HCDR3 comprising the amino acid sequence of the HCDR3 of monoclonal antibody 4237 (e.g., SEQ ID NO: 165), and (ii) a VL comprising: an LCDR1 comprising the amino acid sequence of the LCDR1 of monoclonal antibody 4237 (e.g., SEQ ID NO: 166); an LCDR2 comprising the amino acid sequence of the LCDR2 of monoclonal antibody 4237 (e.g., SEQ ID NO: 167); and an LCDR3 comprising the amino acid sequence of the LCDR3 of monoclonal antibody 4237 (e.g., SEQ ID NO: 168).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 4237 (e.g., SEQ ID NO: 171). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 4237 (e.g., SEQ ID NO: 172).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 4237 (e.g., SEQ ID NO: 171); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 4237 (e.g., SEQ ID NO: 172). In an embodiment, the antibody molecule comprises: (i) a VH comprising the amino acid sequence of the VH of monoclonal antibody 4237 (e.g., SEQ ID NO: 171); and (ii) a VL comprising the amino acid sequence of the VL of monoclonal antibody 4237 (e.g., SEQ ID NO: 172).

In an embodiment the antibody molecule is monoclonal antibody 4237. In an embodiment, monoclonal antibody 4237 is a humanized monoclonal antibody 4237. In an embodiment, the antibody molecule comprises a VH comprising the amino acid sequence of any of SEQ ID NOS: 235-240, a VL comprising the amino acid sequence of any of SEQ ID NOS: 241-245, or both.

In another embodiment, the anti-APRIL antibody molecule:
(i) binds, or substantially binds, to human APRIL;
(ii) inhibits, or substantially inhibits, binding of APRIL (e.g., human APRIL, mouse APRIL, or both) to TACI (e.g., human TACI, mouse TACI, or both);
(iii) inhibits, or substantially inhibits, binding of APRIL (e.g., human APRIL, mouse APRIL, or both) to BCMA (e.g., human BCMA, mouse BCMA, or both); and
(iv) binds, or substantially binds, to one or more, e.g., 2, 3, 4, 5, 6, 7, 8, 9, 10, 11, 12, 13, 14, 15, 16, 17, 18, 19, 20, 21, 22, 23, 24, 25, or more, residues within a region of human APRIL as defined in any of Tables 3-4 or 7, or Table 8 of International Application Publication No. WO2017/091683.

In an embodiment, the antibody molecule binds, or substantially binds, to human APRIL at an EC₅₀ of 20 nM or less, e.g., 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 20 nM, e.g., between 0.01 nM and 20 nM, between 0.1 nM and 20 nM, between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein.

In an embodiment, the antibody molecule does not bind to mouse APRIL, or binds to mouse APRIL with low affinity, e.g., at an EC₅₀ of 1000 nM or more, e.g., 2000 nM or more, e.g., as determined by a method described herein.

In an embodiment, the antibody molecule inhibits, or substantially inhibits, binding of APRIL (e.g., human APRIL, mouse APRIL, or both) to TACI (e.g., human TACI, mouse TACI, or both), at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein.

In an embodiment, the antibody molecule inhibits, or substantially inhibits, binding of APRIL (e.g., human APRIL, mouse APRIL, or both) to BCMA (e.g., human BCMA, mouse BCMA, or both), e.g., at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein.

In an embodiment, the antibody molecule comprises one or both of:
(i) a heavy chain variable region (VH) comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of a monoclonal antibody chosen from antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3125, 2621, 4035, 4035-062, 3934, 4338, 4439, or 4237; an HCDR2 comprising an amino acid sequence that differs by no more than 1, 2, or 3 amino acid residues from, or has at least 85, 90, 95, 99 or 100% homology with, the amino acid sequence of the HCDR2 of the (same) monoclonal antibody; or an HCDR3 comprising an amino acid sequence that differs by no more than 1, 2, or 3 amino acid residues from, or has at least 85, 90, 95, 99 or 100% homology with, the amino acid sequence of the HCDR3 of the (same) monoclonal antibody, or
(ii) a light chain variable region (VL) comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of the (same) monoclonal antibody; an LCDR2 comprising an amino acid sequence that differs by no more than 1, 2, or 3 amino acid residues from, or has at least 85, 90, 95, 99 or 100% homology with, the amino acid sequence of the LCDR2 of the (same) monoclonal antibody; or an LCDR3 comprising an amino acid sequence that differs by no more than 1, 2, or 3 amino acid residues from, or has at least 85, 90, 95, 99 or 100% homology with, the amino acid sequence of the LCDR3 of the (same) monoclonal antibody.

In an embodiment, the antibody molecule comprises one or both of:
(i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of a monoclonal antibody chosen from antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3125, 2621, 4035, 4035-062, 3934, 4338, 4439, or 4237; or
(ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of the (same) monoclonal antibody.

In an embodiment, the antibody molecule is a synthetic antibody molecule. In an embodiment, the antibody molecule is an isolated antibody molecule. In an embodiment, the antibody molecule is a humanized antibody molecule, e.g., comprising one or more framework regions derived from human framework germline sequence.

In an embodiment, the antibody molecule is an IgG antibody molecule, e.g., comprising a heavy chain constant region of IgG, e.g., chosen from IgG1, IgG2 (e.g., IgG2a), IgG3, or IgG4, e.g., IgG2 or IgG4. In an embodiment, the antibody molecule is an IgG1 antibody molecule. In an embodiment, the antibody molecule is an IgG2 antibody molecule. In an embodiment, the antibody molecule comprises a light chain constant region of kappa or lambda light chain.

In an embodiment, the antibody molecule comprises an Fc region. In an embodiment, the Fc region comprises one or more mutations located at the interface between the CH2 and CH3 domains (e.g., to increase the binding affinity to neonatal receptor FcRn and/or the half-life of the antibody molecule).

In an embodiment, the Fc region comprises one or more mutations, e.g., one or more (e.g., 2, 3, 4, 6 or all) mutations chosen from T250Q, M252Y, S254T, T256E, M428L, H433K, N434F, or any combination thereof, of IgG1. In an embodiment, the Fc region comprises one or more mutations at positions 233-236 or 322 of human IgG1 or IgG2, or one or more substitutions at positions 327, 330 or 331 of human IgG4 (e.g., to reduce complement-dependent cytotoxicity (CDC)). In an embodiment, the Fc region comprises one or more (e.g., 2, 3, 4, 6 7 or all) mutations chosen from E233P, L234V, L235A, G236, K322A, A327G, A330S, P331S, or any combination thereof.

In an embodiment, the antibody molecule comprises two heavy chain variable regions and two light chain variable regions. In an embodiment, the antibody molecule is a Fab, F(ab′)2, Fv, Fd, or a single chain Fv fragment (scFv).

In an embodiment, the antibody molecule is a synthetic antibody molecule. In an embodiment, the antibody molecule is an isolated antibody molecule.

In an embodiment, the antibody molecule that comprises the HCDR1, HCDR2, HCDR3, LCDR1, LCDR2, and LCDR3 of any of monoclonal antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 3934, 3833, 3631, 3732, 4338, 4540, 4439, or 4237 comprises a heavy chain variable region and a light chain variable region of any of monoclonal antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 3934, 3833, 3631, 3732, 4338, 4540, 4439, or 4237.

In an embodiment, the antibody molecule that comprises the HCDR1, HCDR2, HCDR3, LCDR1, LCDR2, and LCDR3 of any of monoclonal antibodies 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 3934, 3833, 3631, 3732, 4338, 4540, 4439, or 4237 is monoclonal antibody 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 3934, 3833, 3631, 3732, 4338, 4540, 4439, or 4237.

In an embodiment, the antibody molecule is a humanized monoclonal antibody 2218, 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, 2419-1406, 2922, 3327, 3530, 3525, 3125, 2621, 4035, 4035-062, 3934, 3833, 3631, 3732, 4338, 4540, 4540-063, 4540-033, 4439, or 4237. In an embodiment, the antibody molecule comprises a heavy chain variable region (VH) having an amino acid sequence described in Table 1 or 5. In an embodiment, the antibody molecule comprises a light chain variable region (VL) having an amino acid sequence described in Table 1 or 5. In antibody molecule comprises a heavy chain variable region (VH) having an amino acid sequence described in Table 1 or 5 and a light chain variable region (VL) having an amino acid sequence described in Table 1 or 5.

In an embodiment, the antibody molecule binds, or substantially binds, to human APRIL at an EC₅₀ of 20 nM or less, e.g., 10 nM or less, 9 nM or less or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 100 nM, e.g., between 0.001 nM and 50 nM, between 0.01 nM and 20 nM, between 0.1 nM and 20 nM, e.g., between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein.

In an embodiment, the antibody molecule binds, or substantially binds, to mouse APRIL at an EC₅₀ of 100 nM or less, e.g., 80 nM or less, 60 nM or less, 40 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 100 nM, e.g., between 0.001 nM and 50 nM, between 0.01 nM and 20 nM, between 0.1 nM and 20 nM, e.g., between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein.

In an embodiment, the antibody molecule does not bind, or binds to mouse APRIL with low affinity, e.g., at an EC₅₀ of 1000 nM or more, e.g., 2000 nM or more, e.g., as determined by a method described herein.

In an embodiment, the antibody molecule inhibits, or substantially inhibits, binding of APRIL (e.g., human APRIL, mouse APRIL, or both) to TACI (e.g., human TACI, mouse TACI, or both), inhibits, or substantially inhibits, binding of APRIL (e.g., human APRIL, mouse APRIL, or both) to BCMA (e.g., human BCMA, mouse BCMA, or both), or both.

In an embodiment, the antibody molecule inhibits, or substantially inhibits, binding of APRIL (e.g., human APRIL, mouse APRIL, or both) to TACI (e.g., human TACI, mouse TACI, or both), at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein.

In an embodiment, binding of the antibody molecule to APRIL (e.g., human APRIL) inhibits, or substantially inhibits, the binding of the CRD2 domain of TACI (e.g., human TACI) to APRIL (e.g., human APRIL).

In an embodiment, binding of the antibody molecule to human APRIL inhibits, or substantially inhibits, the binding of human TACI, to one or more, e.g., 2, 3, 4, 5, 6, 7, 8, 9, 10, 11, 12, 13, 14, 15, 16, 17, 18, 19, 20, 21, 22, 23, 24, 25, or all of the human APRIL residues from Table 3. In an embodiment, binding of the antibody molecule to human APRIL, inhibits, or substantially inhibits, the binding of human TACI to one or more, e.g., 2, 3, 4, 5, 6, 7, 8, 9, 10, or all of the human APRIL residues from Table 4. In an embodiment, binding of the antibody molecule to human APRIL, inhibits, or substantially inhibits, the binding of human TACI to one or more, e.g., 2, 3, 4, 5, 6, 7, 8, 9, 10, 11, 12, 13, 14, 15, 16, 17, or all of the human APRIL residues from Table 7. In an embodiment, binding of the antibody molecule to human APRIL, inhibits, or substantially inhibits, the binding of human TACI to one or more, e.g., 2, 3, 4, 5, 6, 7, 8, 9, 10, 11, 12, 13, 14, 15, 16, 17, 18, 19, 20, 21, 22, 23, 24, 25, or all of the human APRIL residues from Table 8 of International Application Publication No. WO2017/091683.

In an embodiment, the antibody molecule inhibits, or substantially inhibits, binding of APRIL (e.g., human APRIL, mouse APRIL, or both) to BCMA (e.g., human BCMA, mouse BCMA, or both).

In an embodiment, the antibody molecule inhibits, or substantially inhibits, binding of APRIL (e.g., human APRIL, mouse APRIL, or both) to BCMA (e.g., human BCMA, mouse BCMA, or both), e.g., at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein.

In an embodiment, the antibody molecule does not inhibit, or does not substantially inhibit, binding of APRIL (e.g., human APRIL, mouse APRIL, or both) to BCMA (e.g., human BCMA, mouse BCMA, or both).

In an embodiment, the antibody molecule is an IgG antibody molecule, e.g., comprising a heavy chain constant region of IgG, e.g., chosen from IgG1, IgG2 (e.g., IgG2a), IgG3, or IgG4, e.g., IgG2 or IgG4. In an embodiment, the antibody molecule is an IgG1 antibody molecule. In another embodiment, the antibody molecule is an IgG2 antibody molecule. In an embodiment, the antibody molecule comprises a light chain constant region of kappa or lambda light chain.

In an embodiment, the antibody molecule comprises an Fc region. In an embodiment, the Fc region comprises one or more mutations located at the interface between the CH2 and CH3 domains (e.g., to increase the binding affinity to neonatal receptor FcRn and/or the half-life of the antibody molecule).

In an embodiment, the Fc region comprises one or more mutations, e.g., one or more (e.g., 2, 3, 4, 6 or all) mutations chosen from T250Q, M252Y, S254T, T256E, M428L, H433K, N434F, or any combination thereof, of IgG1. In an embodiment, the Fc region comprises one or more mutations at positions 233-236 or 322 of human IgG1 or IgG2, or one or more substitutions at positions 327, 330 or 331 of human IgG4 (e.g., to reduce complement-dependent cytotoxicity (CDC)). In an embodiment, the Fc region comprises one or more (e.g., 2, 3, 4, 6 7 or all) mutations chosen from E233P, L234V, L235A, G236, K322A, A327G, A330S, P331S, or any combination thereof.

In an embodiment, the antibody molecule is a humanized antibody molecule, e.g., comprising one or more framework regions derived from human framework germline sequence. In an embodiment, the antibody molecule comprises two heavy chain variable regions and two light chain variable regions. In an embodiment, the antibody molecule is a Fab, F(ab′)2, Fv, Fd, or a single chain Fv fragment (scFv).

In an embodiment, the anti-APRIL antibody molecule is a synthetic, isolated, or humanized anti-APRIL antibody molecule described herein.

In an embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 2218 (e.g., SEQ ID NO: 1 or 7); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 2218 (e.g., SEQ ID NO: 2 or 8); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 2218 (e.g., SEQ ID NO: 3), and
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 2218 (e.g., SEQ ID NO: 4); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 2218 (e.g., SEQ ID NO: 5); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 2218 (e.g., SEQ ID NO: 6).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 2218 (e.g., SEQ ID NO: 9); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 2218 (e.g., SEQ ID NO: 10).

In an embodiment, the antibody molecule comprises a VH encoded by the nucleotide sequence of SEQ ID NO: 71 (or a nucleotide sequence substantially identical thereto) or a VL encoded by the nucleotide sequence of SEQ ID NO: 72 (or a nucleotide sequence substantially identical thereto), or both.

In an embodiment, the antibody molecule is monoclonal antibody 2218. In an embodiment, monoclonal antibody 2218 is humanized monoclonal antibody 2218. In an embodiment, the antibody molecule comprises a VH comprising the amino acid sequence of any of SEQ ID NO: 190-201, a VL comprising the amino acid sequence of any of SEQ ID NO: 202-208, or both.

In an embodiment, the antibody molecule binds, or substantially binds, to human APRIL. In an embodiment, the antibody molecule binds, or substantially binds, to human APRIL at an EC₅₀ of 20 nM or less, e.g., 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 20 nM, e.g., between 0.01 nM and 20 nM, between 0.1 nM and 20 nM, between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein. In an embodiment, the antibody molecule binds to human APRIL at an EC₅₀ of 1 nM or less, e.g., about 0.6 nM. In an embodiment, the antibody molecule does not bind to mouse APRIL, or binds to mouse APRIL with low affinity, e.g., at an EC₅₀ of 1000 nM or more, e.g., 2000 nM or more, e.g., as determined by a method described herein.

In an embodiment, the antibody molecule inhibits, or substantially inhibits, binding of APRIL (e.g., human APRIL) to TACI (e.g., human TACI), BCMA (e.g., human BCMA), or both.

In an embodiment, the antibody molecule inhibits, or substantially inhibits, binding of human APRIL to human TACI at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human TACI at an IC₅₀ of 1 nM or less, e.g., about 0.74 nM.

In an embodiment, the antibody molecule inhibits, or substantially inhibits, binding of human APRIL to human BCMA at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human BCMA at an IC₅₀ of 0.5 nM or less, e.g., about 0.22 nM.

In an embodiment, the antibody molecule comprises one or both of:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence of G-Y-T-F-T-D-Y (SEQ ID NO: 11); an HCDR2 comprising an amino acid sequence of Y-P-L-R-G-S(SEQ ID NO: 12); or an HCCDR3 comprising an amino acid sequence of H-G-A-Y-Y-S-N-A-F-D-Y (SEQ ID NO: 13), or
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence of X1-X2-S-X4-S-V-D-N-D-G-I-R-F-X14-H (SEQ ID NO: 327), wherein X1 is R or K; X2 is A or S; X4 is E or Q; and X14 is M or L; an LCDR2 comprising an amino acid sequence of R-A-S-X4-X5-X6-X7, wherein X4 is N or T; X5 is L or R; X6 is E or A; and X7 is S or T; or an LCDR3 comprising an amino acid sequence of Q-Q-S-N-K-D-P-Y-T (SEQ ID NO: 16).

In another embodiment, the antibody molecule comprises one or both of:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence of D-Y-T-I-H (SEQ ID NO: 17); an HCDR2 comprising an amino acid sequence of W-I-Y-P-L-R-G-S-I-N-Y-X12-X13-X14-F-X16-X17 (SEQ ID NO: 329), wherein X12 is N, S, or A, X13 is E, P, or Q; X14 is K or S; X16 is K or Q; and X17 is D or G; or an HCCDR3 comprising an amino acid sequence of H-G-A-Y-Y-S-N-A-F-D-Y (SEQ ID NO: 13), or
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence of X1-X2-S-X4-S-V-D-N-D-G-I-R-F-X14-H (SEQ ID NO: 327), wherein X1 is R or K; X2 is A or S; X4 is E or Q; and X14 is M or L; an LCDR2 comprising an amino acid sequence of R-A-S-X4-X5-X6-X7, wherein X4 is N or T; X5 is L or R; X6 is E or A; and X7 is S or T; or an LCDR3 comprising an amino acid sequence of Q-Q-S-N-K-D-P-Y-T (SEQ ID NO: 16).

In an embodiment, the antibody molecule is any of antibodies 2419, 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, or 2419-1406.

In an embodiment, the antibody molecule binds, or substantially binds, to human APRIL at an EC₅₀ of 20 nM or less, e.g., 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 20 nM, e.g., between 0.01 nM and 20 nM, between 0.1 nM and 20 nM, between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein. In an embodiment, the antibody molecule binds to human APRIL at an EC₅₀ of 0.01 nM or less, e.g., about 0.001-0.005 nM or 0.002-0.004 nM, e.g., about 0.001, 0.002, 0.003, 0.004, or 0.005 nM. In an embodiment, the antibody molecule does not bind to mouse APRIL, or binds to mouse APRIL with low affinity, e.g., at an EC₅₀ of 1000 nM or more, e.g., 2000 nM or more, e.g., as determined by a method described herein.

In an embodiment, the antibody molecule inhibits, or substantially inhibits, binding of APRIL (e.g., human APRIL) to TACI (e.g., human TACI), BCMA (e.g., human BCMA), or both.

In an embodiment, the antibody molecule inhibits, or substantially inhibits, binding of human APRIL to human TACI at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human TACI at an IC₅₀ of 0.5 nM or less, e.g., about 0.1-0.5 nM or 0.2-0.4 nM, e.g., about 0.1, 0.2, 0.3, 0.4, or 0.5 nM.

In an embodiment, the antibody molecule inhibits, or substantially inhibits, binding of human APRIL to human BCMA at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human BCMA at an IC₅₀ of 0.5 nM or less, e.g., about 0.1-0.5 nM or 0.2-0.4 nM, e.g., about 0.1, 0.2, 0.3, 0.4, or 0.5 nM.

In another embodiment, the antibody molecule comprises (i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 2419 (e.g., SEQ ID NO: 11 or 17); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 2419 (e.g., SEQ ID NO: 12 or 18); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 2419 (e.g., SEQ ID NO: 13), and
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 2419 (e.g., SEQ ID NO: 14); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 2419 (e.g., SEQ ID NO: 15); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 2419 (e.g., SEQ ID NO: 16).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 2419 (e.g., SEQ ID NO: 19); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 2419 (e.g., SEQ ID NO: 20).

In an embodiment, the antibody molecule comprises a VH encoded by the nucleotide sequence of SEQ ID NO: 73 (or a nucleotide sequence substantially identical thereto) or a VL encoded by the nucleotide sequence of SEQ ID NO: 74 (or a nucleotide sequence substantially identical thereto), or both.

In an embodiment, the antibody molecule is monoclonal antibody 2419. In an embodiment, monoclonal antibody 2419 is humanized monoclonal antibody 2419. In an embodiment, the antibody molecule comprises a VH comprising the amino acid sequence of any of SEQ ID NOS: 209-214, a VL comprising the amino acid sequence of any of SEQ ID NOS: 215-219, or both.

In an embodiment, the antibody molecule binds, or substantially binds, to human APRIL at an EC₅₀ of 20 nM or less, e.g., 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 20 nM, e.g., between 0.01 nM and 20 nM, between 0.1 nM and 20 nM, between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein. In an embodiment, the antibody molecule binds to human APRIL at an EC₅₀ of 1 nM or less, e.g., about 0.8 nM, about 0.003 nM, or about 0.002 nM. In an embodiment, the antibody molecule does not bind, or bind to mouse APRIL with low affinity, e.g., at an EC₅₀ of 500 nM or more.

In an embodiment, the antibody molecule inhibits, or substantially inhibits, binding of APRIL (e.g., human APRIL) to TACI (e.g., human TACI), BCMA (e.g., human BCMA), or both.

In an embodiment, the antibody molecule inhibits, or substantially inhibits, binding of human APRIL to human TACI, at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human TACI at an IC₅₀ of 1 nM or less, e.g., about 0.74 nM, about 0.4 nM, 0.3 nM, or 0.2 nM.

In an embodiment, the antibody molecule inhibits, or substantially inhibits, binding of human APRIL to human BCMA, at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human BCMA at an IC₅₀ of 5 nM or less, e.g., about 4 nM, about 2 nM, or about 1 nM, or 0.5 nM or less, e.g., about 0.22 nM, about 1 nM, about 0.7 nM, about 0.3 nM, about 0.2 nM, or about 0.1 nM.

In another embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of a 2419-related antibody (e.g., SEQ ID NO: 11 or 17); an HCDR2 comprising an amino acid sequence that differs by no more than 1, 2, or 3 amino acid residues from, or has at least 85, 90, 95, 99 or 100% homology with, the amino acid sequence of the HCDR2 of the 2419-related antibody (e.g., SEQ ID NOS: 12, 282, 287, or 290); or an HCDR3 comprising an amino acid sequence that differs by no more than 1, 2, or 3 amino acid residues from, or has at least 85, 90, 95, 99 or 100% homology with, the amino acid sequence of the HCDR3 of the 2419-related antibody (e.g., SEQ ID NO: 13), and
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of the 2419-related antibody (e.g., SEQ ID NOS: 280 or 314); an LCDR2 comprising an amino acid sequence that differs by no more than 1, 2, or 3 amino acid residues from, or has at least 85, 90, 95, 99 or 100% homology with, the amino acid sequence of the LCDR2 of the 2419-related antibody (e.g., SEQ ID NOS: 281, 285, 293, or 315); or an LCDR3 comprising an amino acid sequence that differs by no more than 1, 2, or 3 amino acid residues from, or has at least 85, 90, 95, 99 or 100% homology with, the amino acid sequence of the LCDR3 of the 2419-related antibody (e.g., SEQ ID NO: 16).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of the 2419-related antibody (e.g., SEQ ID NOS: 283, 288, 289, 291, 292, 294, 296, or 317); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of the 2419-related antibody (e.g., SEQ ID NOS: 284, 286, 295, or 316).

In an embodiment, the antibody molecule comprises a VH encoded by the VH nucleotide sequence of the 2419-related antibody (e.g., SEQ ID NOS: 304, 307, 308, 309, 310, 311, 313, or 319) (or a nucleotide sequence substantially identical thereto) or a VL encoded by the VL nucleotide sequence of the 2419-related antibody (e.g., SEQ ID NOS: 305, 306, 312, or 318) (or a nucleotide sequence substantially identical thereto), or both.

In an embodiment, the 2419-related antibody molecule is chosen from antibodies 2419-0105, 2419-0205, 2419-0206, 2419-0406, 2419-0605, 2419-0805, 2419-0806, 2419-1204, 2419-1205, 2419-1210, 2419-1305, 2419-1306, 2419-1310, or 2419-1406. In an embodiment, the 2419-related antibody is humanized antibody molecule. In an embodiment, the antibody molecule comprises a VH comprising the amino acid sequence of any of SEQ ID NOS: 209-214, 283, 288, 289, 291, 292, 294, 296, or 317, a VL comprising the amino acid sequence of any of SEQ ID NOS: 215-219, 284, 286, 295, or 316, or both.

In an embodiment, the antibody molecule binds, or substantially binds, to human APRIL. In an embodiment, the antibody molecule binds, or substantially binds, to human APRIL at an EC₅₀ of 20 nM or less, e.g., 10 nM or less, 9 nM or less or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 20 nM, e.g., between 0.01 nM and 20 nM, between 0.1 nM and 20 nM, e.g., between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein. In an embodiment, the antibody molecule binds to human APRIL at an EC₅₀ of 1 nM or less, e.g., about 0.8 nM, about 0.003 nM, or about 0.002 nM.

In an embodiment, the antibody molecule does not bind, or bind to mouse APRIL with low affinity, e.g., at an EC₅₀ of 500 nM or more.

In an embodiment, the antibody molecule inhibits, or substantially inhibits, binding of APRIL (e.g., human APRIL) to TACI (e.g., human TACI), BCMA (e.g., human BCMA), or both. In an embodiment, the antibody molecule inhibits, or substantially inhibits, binding of human APRIL to human TACI, at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human TACI at an IC₅₀ of 1 nM or less, e.g., about 0.74 nM, about 0.4 nM, 0.3 nM, or 0.2 nM.

In an embodiment, the antibody molecule inhibits, or substantially inhibits, binding of human APRIL to human BCMA, at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human BCMA at an IC₅₀ of 5 nM or less, e.g., about 4 nM, about 2 nM, or about 1 nM, or 0.5 nM or less, e.g., about 0.22 nM, about 1 nM, about 0.7 nM, about 0.3 nM, about 0.2 nM, or about 0.1 nM.

In another embodiment, the antibody molecule comprises (i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 2922 (e.g., SEQ ID NO: 21 or 37); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 2922 (e.g., SEQ ID NO: 32 or 38); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 2922 (e.g., SEQ ID NO: 33), and
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 2922 (e.g., SEQ ID NO: 34); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 2922 (e.g., SEQ ID NO: 35); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 2922 (e.g., SEQ ID NO: 36).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 2922 (e.g., SEQ ID NO: 39); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 2922 (e.g., SEQ ID NO: 40).

In an embodiment, the antibody molecule comprises a VH encoded by the nucleotide sequence of SEQ ID NO: 77 (or a nucleotide sequence substantially identical thereto) or a VL encoded by the nucleotide sequence of SEQ ID NO: 78 (or a nucleotide sequence substantially identical thereto), or both.

In an embodiment, the antibody molecule is monoclonal antibody 2922. In an embodiment, the antibody molecule is humanized monoclonal antibody 2922.

In an embodiment, the antibody molecule binds, or substantially binds, to human APRIL. In an embodiment, the antibody molecule binds, or substantially binds, to human APRIL at an EC₅₀ of 20 nM or less, e.g., 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 20 nM, e.g., between 0.01 nM and 20 nM, between 0.1 nM and 20 nM, between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein. In an embodiment, the antibody molecule binds to human APRIL at an EC₅₀ of 5 nM or less, e.g., about 3.3 nM. In an embodiment, the antibody molecule does not bind, or binds to mouse APRIL with low affinity, e.g., at an EC₅₀ of 1000 nM or more, e.g., 2000 nM or more, e.g., as determined by a method described herein.

In an embodiment, the antibody molecule inhibits, or substantially inhibits, binding of APRIL (e.g., human APRIL) to TACI (e.g., human TACI), BCMA (e.g., human BCMA), or both. In an embodiment, the antibody molecule inhibits, or substantially inhibits, binding of human APRIL to human TACI, at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human TACI at an IC₅₀ of 50 nM or less, e.g., about 31.64 nM.

In an embodiment, the antibody molecule inhibits, or substantially inhibits, binding of human APRIL to human BCMA, at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the IC₅₀ is 50 nM or less. In an embodiment, the antibody molecule inhibits binding of human TACI to human BCMA at an IC₅₀ of 25 nM or less, e.g., about 21.96 nM.

In another embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3327 (e.g., SEQ ID NO: 51 or 57); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3327 (e.g., SEQ ID NO: 52 or 58); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3327 (e.g., SEQ ID NO: 53), and
(ii) a light chain variable region (VH), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3327 (e.g., SEQ ID NO: 54); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3327 (e.g., SEQ ID NO: 55); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3327 (e.g., SEQ ID NO: 56).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3327 (e.g., SEQ ID NO: 59); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3327 (e.g., SEQ ID NO: 60).

In an embodiment, the antibody molecule comprises a VH encoded by the nucleotide sequence of SEQ ID NO: 81 (or a nucleotide sequence substantially identical thereto) or a VL encoded by the nucleotide sequence of SEQ ID NO: 82 (or a nucleotide sequence substantially identical thereto), or both.

In an embodiment, the antibody molecule is monoclonal antibody 3327. In an embodiment, the antibody molecule is humanized antibody 3327.

In an embodiment, the antibody molecule binds, or substantially binds, to human APRIL. In an embodiment, the antibody molecule binds, or substantially binds, to human APRIL at an EC₅₀ of 100 nM or less, e.g., 80 nM or less, 60 nM or less, 40 nM or less, 20 nM or less, 10 nM or less, 9 nM or less 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 100 nM, e.g., between 0.001 nM and 50 nM, between 0.01 nM and 20 nM, between 0.1 nM and 10 nM, between 0.5 nM and 5 nM or between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein. In an embodiment, the antibody molecule does not bind, or binds to mouse APRIL with low affinity, e.g., at an EC₅₀ of 1000 nM or more, e.g., 2000 nM or more, e.g., as determined by a method described herein.

In an embodiment, the antibody molecule inhibits, or substantially inhibits, binding of APRIL (e.g., human APRIL) to TACI (e.g., human TACI), BCMA (e.g., human BCMA), or both. In an embodiment, the antibody molecule inhibits, or substantially inhibits, binding of human APRIL to human TACI, at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human TACI at an IC₅₀ of 5 nM or less, e.g., about 3.16 nM.

In an embodiment, the antibody molecule inhibits, or substantially inhibits, binding of human APRIL to human BCMA, at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the IC₅₀ is 50 nM or less. In an embodiment, the antibody molecule inhibits binding of human APRIL to human BCMA at an IC₅₀ of 5 nM or less, e.g., about 2.35 nM.

In another embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4035 (e.g., SEQ ID NO: 93 or 99); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4035 (e.g., SEQ ID NO: 94 or 100); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4035 (e.g., SEQ ID NO: 95), and
(ii) a light chain variable region (VH), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4035 (e.g., SEQ ID NO: 96); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4035 (e.g., SEQ ID NO: 97); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4035 (e.g., SEQ ID NO: 98).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 4035 (e.g., SEQ ID NO: 101); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 4035 (e.g., SEQ ID NO: 102).

In an embodiment, the antibody molecule comprises a VH encoded by the nucleotide sequence of SEQ ID NO: 173 (or a nucleotide sequence substantially identical thereto) or a VL encoded by the nucleotide sequence of SEQ ID NO: 174 (or a nucleotide sequence substantially identical thereto), or both.

In an embodiment, the antibody molecule is monoclonal antibody 4035. In an embodiment, monoclonal antibody 4035 is humanized monoclonal antibody 4035. In an embodiment, the antibody molecule comprises a VH comprising the amino acid sequence of any of SEQ ID NO: 220-227 or 262-265, a VL comprising the amino acid sequence of any of SEQ ID NO: 228-234, or both.

In an embodiment, the antibody molecule binds, or substantially binds, to human APRIL. In an embodiment, the antibody molecule binds, or substantially binds, to human APRIL at an EC₅₀ of 20 nM or less, e.g., 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 20 nM, e.g., between 0.01 nM and 20 nM, between 0.1 nM and 20 nM, between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein. In an embodiment, the antibody molecule binds to human APRIL at an EC₅₀ of 0.01 nM or less, e.g., about 0.001-0.002 nM. In an embodiment, the antibody molecule does not bind, or binds to mouse APRIL with low affinity, e.g., at an EC₅₀ of 1000 nM or more, e.g., 2000 nM or more, e.g., as determined by a method described herein.

In an embodiment, the antibody molecule inhibits, or substantially inhibits, binding of APRIL (e.g., human APRIL) to TACI (e.g., human TACI), BCMA (e.g., human BCMA), or both. In an embodiment, the antibody molecule inhibits, or substantially inhibits, binding of human APRIL to human TACI, at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human TACI at an IC₅₀ of 5 nM or less, e.g., about 3.16 nM, or about 0.1-0.5 nM or 0.2-0.4 nM.

In an embodiment, the antibody molecule inhibits, or substantially inhibits, binding of human APRIL to human BCMA, at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human BCMA at an IC₅₀ of 5 nM or less, e.g., about 2.35 nM, or about 0.1-0.5 nM or 0.1-0.2 nM.

In another embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4035-062 (e.g., SEQ ID NO: 93 or 99); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4035-062 (e.g., SEQ ID NO: 94 or 273); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4035-062 (e.g., SEQ ID NO: 95), and
(ii) a light chain variable region (VH), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4035-062 (e.g., SEQ ID NO: 96); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4035-062 (e.g., SEQ ID NO: 97); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4035-062 (e.g., SEQ ID NO: 98).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 4035-062 (e.g., SEQ ID NO: 225); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 4035-062 (e.g., SEQ ID NO: 229).

In an embodiment, the antibody molecule comprises a VH encoded by the nucleotide sequence of SEQ ID NO: 299 (or a nucleotide sequence substantially identical thereto) or a VL encoded by the nucleotide sequence of SEQ ID NO: 300 (or a nucleotide sequence substantially identical thereto), or both.

In an embodiment, the antibody molecule is monoclonal antibody 4035-062.

In an embodiment, the antibody molecule binds, or substantially binds, to human APRIL. In an embodiment, the antibody molecule binds, or substantially binds, to human APRIL at an EC₅₀ of 20 nM or less, e.g., 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 20 nM, e.g., between 0.01 nM and 20 nM, between 0.1 nM and 20 nM, between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein. In an embodiment, the antibody molecule binds to human APRIL at an EC₅₀ of 0.01 nM or less, e.g., about 0.001-0.002 nM. In an embodiment, the antibody molecule does not bind, or binds to mouse APRIL with low affinity, e.g., at an EC₅₀ of 1000 nM or more, e.g., 2000 nM or more, e.g., as determined by a method described herein.

In an embodiment, the antibody molecule inhibits, or substantially inhibits, binding of APRIL (e.g., human APRIL) to TACI (e.g., human TACI), BCMA (e.g., human BCMA), or both. In an embodiment, the antibody molecule inhibits, or substantially inhibits, binding of human APRIL to human TACI, at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human TACI at an IC₅₀ of 1 nM or less, e.g., about 0.1-0.5 nM or 0.2-0.4 nM.

In an embodiment, the antibody molecule inhibits, or substantially inhibits, binding of human APRIL to human BCMA, at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human BCMA at an IC₅₀ of 1 nM or less, e.g., about 0.1-0.5 nM or 0.1-0.2 nM.

In another embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising the amino acid sequence of I-Y-D-V-H (SEQ ID NO: 99); an HCDR2 comprising the amino acid sequence of V-I-W-S-D-G-S-T-D-Y-N-X12-X13-X14-X15-S(SEQ ID NO: 342), X12 is A or P, X13 is A or S, X14 is F or L, and X15 is I or K; or an HCDR3 comprising the amino acid sequence of N-W-V-D-Q-A-W-F-A-Y (SEQ ID NO: 95), and
(ii) a light chain variable region (VH), wherein the light chain variable region comprises three light chain complementarity determining regions (LCDR1, LCDR2, and LCDR3), wherein the light chain variable region comprises one, two, or all of the following: an LCDR1 comprising the amino acid sequence of R-A-S-K-N-I-Y-S-Y-L-A (SEQ ID NO: 96); an LCDR2 comprising the amino acid sequence of N-A-K-T-L-P-E (SEQ ID NO: 97); or an LCDR3 comprising the amino acid sequence of Q-H-H-Y-G-T-P-L-T (SEQ ID NO: 98).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of SEQ ID NO: 101 or 225; or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of SEQ ID NO: 102 or 229.

In an embodiment, the antibody molecule is monoclonal antibody 4035. In an embodiment, the antibody molecule is monoclonal antibody 4035-062.

In another embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3934 (e.g., SEQ ID NO: 103 or 109); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3934 (e.g., SEQ ID NO: 104 or 110); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3934 (e.g., SEQ ID NO: 105), and
(ii) a light chain variable region (VH), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3934 (e.g., SEQ ID NO: 106); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3934 (e.g., SEQ ID NO: 107); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3934 (e.g., SEQ ID NO: 108).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3934 (e.g., SEQ ID NO: 111); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3934 (e.g., SEQ ID NO: 112).

In an embodiment, the antibody molecule comprises a VH encoded by the nucleotide sequence of SEQ ID NO: 175 (or a nucleotide sequence substantially identical thereto) or a VL encoded by the nucleotide sequence of SEQ ID NO: 176 (or a nucleotide sequence substantially identical thereto), or both.

In an embodiment, the antibody molecule is monoclonal antibody 3934. In an embodiment, the antibody molecule is humanized monoclonal antibody 3934.

In an embodiment, the antibody molecule binds, or substantially binds, to human APRIL. In an embodiment, the antibody molecule binds, or substantially binds, to human APRIL at an EC₅₀ of 20 nM or less, e.g., 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 20 nM, e.g., between 0.01 nM and 20 nM, between 0.1 nM and 20 nM, between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein. In an embodiment, the antibody molecule does not bind, or binds to mouse APRIL with low affinity, e.g., at an EC₅₀ of 1000 nM or more, e.g., 2000 nM or more, e.g., as determined by a method described herein.

In an embodiment, the antibody molecule inhibits, or substantially inhibits, binding of APRIL (e.g., human APRIL) to TACI (e.g., human TACI), BCMA (e.g., human BCMA), or both. In an embodiment, the antibody molecule inhibits, or substantially inhibits, binding of human APRIL to human TACI, at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human TACI at an IC₅₀ of 5 nM or less, e.g., about 3.16 nM.

In an embodiment, the antibody molecule inhibits, or substantially inhibits, binding of human APRIL to human BCMA, at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human BCMA at an IC₅₀ of 5 nM or less, e.g., about 2.35 nM.

In an embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4338 (e.g., SEQ ID NO: 11 or 149); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4338 (e.g., SEQ ID NO: 142 or 150); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4338 (e.g., SEQ ID NO: 143), and
(ii) a light chain variable region (VH), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4338 (e.g., SEQ ID NO: 144 or 146); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4338 (e.g., SEQ ID NO: 107 or 147); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4338 (e.g., SEQ ID NO: 145 or 148).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 4338 (e.g., SEQ ID NO: 151); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 4338 (e.g., SEQ ID NO: 152 or 153).

In an embodiment, the antibody molecule comprises a VH encoded by the nucleotide sequence of SEQ ID NO: 183 (or a nucleotide sequence substantially identical thereto) or a VL encoded by the nucleotide sequence of SEQ ID NO: 184 or 185 (or a nucleotide sequence substantially identical thereto), or both.

In an embodiment, the antibody molecule is monoclonal antibody 4338. In an embodiment, the antibody molecule is humanized monoclonal antibody 4338.

In an embodiment, the antibody molecule binds, or substantially binds, to human APRIL. In an embodiment, the antibody molecule binds, or substantially binds, to human APRIL at an EC₅₀ of 20 nM or less, e.g., 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 20 nM, e.g., between 0.01 nM and 20 nM, between 0.1 nM and 20 nM, between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein. In an embodiment, the antibody molecule does not bind, or binds to mouse APRIL with low affinity, e.g., at an EC₅₀ of 1000 nM or more, e.g., 2000 nM or more, e.g., as determined by a method described herein.

In an embodiment, the antibody molecule inhibits, or substantially inhibits, binding of APRIL (e.g., human APRIL) to TACI (e.g., human TACI), BCMA (e.g., human BCMA), or both. In an embodiment, the antibody molecule inhibits, or substantially inhibits, binding of human APRIL to human TACI, at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human TACI at an IC₅₀ of 5 nM or less, e.g., about 3.16 nM.

In an embodiment, the antibody molecule inhibits, or substantially inhibits, binding of human APRIL to human BCMA, at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human BCMA at an IC₅₀ of 5 nM or less, e.g., about 2.35 nM.

In another embodiment, the antibody molecule comprises (i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4237 (e.g., SEQ ID NO: 163 or 169); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4237 (e.g., SEQ ID NO: 164 or 170); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4237 (e.g., SEQ ID NO: 165), and
(ii) a light chain variable region (VH), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4237 (e.g., SEQ ID NO: 166); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4237 (e.g., SEQ ID NO: 167); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4237 (e.g., SEQ ID NO: 168).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 4237 (e.g., SEQ ID NO: 171); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 4237 (e.g., SEQ ID NO: 172).

In an embodiment, the antibody molecule comprises a VH encoded by the nucleotide sequence of SEQ ID NO: 188 (or a nucleotide sequence substantially identical thereto) or a VL encoded by the nucleotide sequence of SEQ ID NO: 189 (or a nucleotide sequence substantially identical thereto), or both.

In an embodiment, the antibody molecule is monoclonal antibody 4237. In an embodiment, monoclonal antibody 4237 is humanized monoclonal antibody 4237. In an embodiment, the antibody molecule comprises a VH comprising the amino acid sequence of any of SEQ ID NO: 235-240, a VL comprising the amino acid sequence of any of SEQ ID NO: 241-245, or both.

In an embodiment, the antibody molecule binds, or substantially binds, to human APRIL. In an embodiment, the antibody molecule binds, or substantially binds, to human APRIL at an EC₅₀ of 20 nM or less, e.g., 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 20 nM, e.g., between 0.01 nM and 20 nM, between 0.1 nM and 20 nM, between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein. In an embodiment, the antibody molecule does not bind, or binds to mouse APRIL with low affinity, e.g., at an EC₅₀ of 1000 nM or more, e.g., 2000 nM or more, e.g., as determined by a method described herein.

In an embodiment, the antibody molecule inhibits, or substantially inhibits, binding of APRIL (e.g., human APRIL) to TACI (e.g., human TACI), BCMA (e.g., human BCMA), or both. In an embodiment, the antibody molecule inhibits, or substantially inhibits, binding of human APRIL to human TACI, at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human TACI at an IC₅₀ of 5 nM or less, e.g., about 3.16 nM.

In an embodiment, the antibody molecule inhibits, or substantially inhibits, binding of human APRIL to human BCMA, at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human BCMA at an IC₅₀ of 5 nM or less, e.g., about 2.35 nM.

In another embodiment, the antibody molecule comprises one or both of:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence of G-Y-X3-X4-T-X6-X7-Y (SEQ ID NO: 330), wherein X3 is S or T; X4 is I or F; X6 is S or absent; and X7 is G, D or S; an HCDR2 comprising an amino acid sequence of X3-X4-X5-X6-X7-X8, wherein X3 is absent, N or Y; X4 is S or P, X5 is Y, L or R; X6 is D, N or R; X7 is G or S; and X8 is Y, D or S; or an HCCDR3 comprising an amino acid sequence of X1-X2-X3-X4-Y-X6-X7-X8-X9-F-X11-X12, wherein X1 is Y, E or H; X2 is absent or G; X3 is Y, D or A; X4 is D, G or Y; X6 is E, absent or D; X7 is D, Y, S or K; X8 is W, N or R; X9 is Y, A or G; X1I is G or D; and X12 is V or Y, or (ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence of X1-A-S-X4-S-V-X7-X8-X9-G-X11-X12-X13-X14-X15 (SEQ ID NO: 333), wherein X1 is R or K; X4 is E or Q; X7 is D or S; X8 is N, F, I or N; X9 is Y, A, I or D; X1I is I or T; X12 is S, N or R; X13 is F, L or S; X14 is M or I; and X15 is N or H; an LCDR2 comprising an amino acid sequence of X1-A-S-N-X5-X6-X7, wherein X1 is A, R or H; X5 is Q or L; X6 is G or E; and X7 is S, P or T; or an LCDR3 comprising an amino acid sequence of X1-Q-S-X4-X5-X6-P-X8-T (SEQ ID NO: 335), wherein X1 is Q or L; X4 is K, R or N; X5 is E or K; X6 is V, Y, I or D; and X8 is R, W or Y.

In an embodiment, the antibody molecule is any of monoclonal antibodies 2218, 2419, 2922, or 3327.

In an embodiment, the antibody molecule binds, or substantially binds, to human APRIL. In an embodiment, the antibody molecule binds, or substantially binds, to human APRIL at an EC₅₀ of 20 nM or less, e.g., 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 20 nM, e.g., between 0.01 nM and 20 nM, between 0.1 nM and 20 nM, between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein. In an embodiment, the antibody molecule does not bind, or binds to mouse APRIL with low affinity, e.g., at an EC₅₀ of 1000 nM or more, e.g., 2000 nM or more, e.g., as determined by a method described herein.

In an embodiment, the antibody molecule inhibits, or substantially inhibits, binding of APRIL (e.g., human APRIL) to TACI (e.g., human TACI), BCMA (e.g., human BCMA), or both. In an embodiment, the antibody molecule inhibits, or substantially inhibits, binding of human APRIL to human TACI, at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, or 0.1 nM or less, e.g., between 0.1 and 50 nM, e.g., between 0.1 nM and 25 nM, between 0.1 nM and 10 nM, between 0.5 nM and 5 nM, or between 1 nM and 5 nM, e.g., as determined by a method described herein.

In an embodiment, the antibody molecule inhibits, or substantially inhibits, binding of human APRIL to human BCMA, at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein.

In another embodiment, the antibody molecule comprises one or both of:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence of X6-X7-Y-X9-X10-X11, wherein X6 is S or absent; X7 is G, D or S; X9 is Y, F, T or D; X10 is W, M, I or V; and X1I is N, H or F; an HCDR2 comprising an amino acid sequence of X1-I-X3-X4-X5-X6-X7-X8-X9-X10-Y-N-X13-X14-X15-K-X17 (SEQ ID NO: 337), wherein X1 is Y, R or W; X3 is absent, N or Y; X4 is S or P, X5 is Y, L or R; X6 is D, N or R; X7 is G or S; X8 is Y, D or S; X9 is N, T or I; X10 is N, For K; X13 is P, Q or E; X14 is S or K; X15 is L or F; and X17 is N, G or D; or an HCCDR3 comprising an amino acid sequence of X1-X2-X3-X4-Y-X6-X7-X8-X9-F-X1 1-X12, wherein X1 is Y, E or H; X2 is absent or G; X3 is Y, D or A; X4 is D, G or Y; X6 is E, absent or D; X7 is D, Y, S or K; X8 is W, N or R; X9 is Y, A or G; X1I is G or D; and X12 is V or Y, or
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence of X1-A-S-X4-S-V-X7-X8-X9-G-X11-X12-X13-X14-X15 (SEQ ID NO: 333), wherein X1 is R or K; X4 is E or Q; X7 is D or S; X8 is N, F, I or N; X9 is Y, A, I or D; X1I is I or T; X12 is S, N or R; X13 is F, L or S; X14 is M or I; and X15 is N or H; an LCDR2 comprising an amino acid sequence of X1-A-S-N-X5-X6-X7, wherein X1 is A, R or H; X5 is Q or L; X6 is G or E; and X7 is S, P or T; or an LCDR3 comprising an amino acid sequence of X1-Q-S-X4-X5-X6-P-X8-T (SEQ ID NO: 335), wherein Xi is Q or L; X4 is K, R or N; X5 is E or K; X6 is V, Y, I or D; and X8 is R, W or Y.

In an embodiment, the antibody molecule is any of monoclonal antibodies 2218, 2419, 2922, or 3327. In an embodiment, the antibody molecule is a humanized monoclonal antibody 2218, 2419, 2922, or 3327.

In an embodiment, the antibody molecule binds, or substantially binds, to human APRIL. In an embodiment, the antibody molecule binds, or substantially binds, to human APRIL at an EC₅₀ of 20 nM or less, e.g., 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 20 nM, e.g., between 0.01 nM and 20 nM, between 0.1 nM and 20 nM, between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein. In an embodiment, the antibody molecule does not bind, or binds to mouse APRIL with low affinity, e.g., at an EC₅₀ of 1000 nM or more, e.g., 2000 nM or more, e.g., as determined by a method described herein.

In an embodiment, the antibody molecule inhibits, or substantially inhibits, binding of APRIL (e.g., human APRIL) to TACI (e.g., human TACI), BCMA (e.g., human BCMA), or both. In an embodiment, the antibody molecule inhibits, or substantially inhibits, binding of human APRIL to human TACI, at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein.

In an embodiment, the antibody molecule inhibits, or substantially inhibits, binding of human APRIL to human BCMA, at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein.

In another embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3530 (e.g., SEQ ID NO: 61 or 64); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3530 (e.g., SEQ ID NO: 62 or 65); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3530 (e.g., SEQ ID NO: 63), and
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3530 (e.g., SEQ ID NO: 67); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3530 (e.g., SEQ ID NO: 45); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3530 (e.g., SEQ ID NO: 46).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3530 (e.g., SEQ ID NO: 66); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3530 (e.g., SEQ ID NO: 70).

In an embodiment, the antibody molecule comprises a VH encoded by the nucleotide sequence of SEQ ID NO: 83 (or a nucleotide sequence substantially identical thereto) or a VL encoded by the nucleotide sequence of SEQ ID NO: 84 (or a nucleotide sequence substantially identical thereto), or both. In an embodiment, the antibody molecule is monoclonal antibody 3530. In an embodiment, the antibody molecule is a humanized monoclonal antibody 3530.

In an embodiment, the antibody molecule binds, or substantially binds, to human APRIL, mouse APRIL, or both.

In an embodiment, the antibody molecule binds, or substantially binds, to human APRIL at an EC₅₀ of 20 nM or less, e.g., 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 20 nM, e.g., between 0.01 nM and 20 nM, between 0.1 nM and 20 nM, between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein. In an embodiment, the antibody molecule binds to human APRIL at an EC₅₀ of 5 nM or less, e.g., about 2.7 nM.

In an embodiment, the antibody molecule binds, or substantially binds, to mouse APRIL at an EC₅₀ of 100 nM or less, e.g., 80 nM or less, 60 nM or less, 40 nM or less, 20 nM or less, 10 nM or less, 9 nM or less 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 100 nM, e.g., between 0.001 nM and 50 nM, between 0.01 nM and 20 nM, between 0.1 nM and 10 nM, between 0.5 nM and 5 nM or between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein.

In an embodiment, the antibody molecule inhibits, or substantially inhibits, binding of APRIL (e.g., human APRIL) to TACI (e.g., human TACI), BCMA (e.g., human BCMA), or both. In an embodiment, the antibody molecule inhibits, or substantially inhibits, binding of APRIL (e.g., human APRIL, mouse APRIL, or both) to TACI (e.g., human TACI, mouse TACI, or both), at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human TACI at an IC₅₀ of 5 nM or less, e.g., about 4.95 nM.

In an embodiment, the antibody molecule inhibits, or substantially inhibits, binding of APRIL (e.g., human APRIL, mouse APRIL, or both) to BCMA (e.g., human BCMA, mouse BCMA, or both), at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human BCMA at an IC₅₀ of 1 nM or less, e.g., about 0.68 nM.

In an embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3525 (e.g., SEQ ID NO: 61 or 64); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3525 (e.g., SEQ ID NO: 62 or 65); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3525 (e.g., SEQ ID NO: 63), and
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3525 (e.g., SEQ ID NO: 44); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3525 (e.g., SEQ ID NO: 45); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3525 (e.g., SEQ ID NO: 46).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3525 (e.g., SEQ ID NO: 66); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3525 (e.g., SEQ ID NO: 50).

In an embodiment, the antibody molecule comprises a VH encoded by the nucleotide sequence of SEQ ID NO: 83 (or a nucleotide sequence substantially identical thereto) or a VL encoded by the nucleotide sequence of SEQ ID NO: 80 (or a nucleotide sequence substantially identical thereto), or both.

In an embodiment, the antibody molecule is monoclonal antibody 3525. In an embodiment, the antibody molecule is a humanized monoclonal antibody 3525.

In an embodiment, the antibody molecule binds, or substantially binds, to human APRIL, mouse APRIL, or both.

In an embodiment, the antibody molecule binds, or substantially binds, to human APRIL at an EC₅₀ of 20 nM or less, e.g., 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 20 nM, e.g., between 0.01 nM and 20 nM, between 0.1 nM and 20 nM, between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein. In an embodiment, the antibody molecule binds to human APRIL at an EC₅₀ of 5 nM or less, e.g., about 2.5 nM.

In an embodiment, the antibody molecule binds, or substantially binds, to mouse APRIL at an EC₅₀ of 100 nM or less, e.g., 80 nM or less, 60 nM or less, 40 nM or less, 20 nM or less, 10 nM or less, 9 nM or less 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 100 nM, e.g., between 0.001 nM and 50 nM, between 0.01 nM and 20 nM, between 0.1 nM and 10 nM, between 0.5 nM and 5 nM or between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein.

In an embodiment, the antibody molecule inhibits, or substantially inhibits, binding of APRIL (e.g., human APRIL) to TACI (e.g., human TACI), BCMA (e.g., human BCMA), or both. In an embodiment, the antibody molecule inhibits, or substantially inhibits, binding of APRIL (e.g., human APRIL, mouse APRIL, or both) to TACI (e.g., human TACI, mouse TACI, or both), at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human TACI at an IC₅₀ of 5 nM or less, e.g., about 4.05 nM.

In an embodiment, the antibody molecule inhibits, or substantially inhibits, binding of APRIL (e.g., human APRIL, mouse APRIL, or both) to BCMA (e.g., human BCMA, mouse BCMA, or both), at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human BCMA at an IC₅₀ of 1 nM or less, e.g., about 0.85 nM.

In an embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3833 (e.g., SEQ ID NO: 113 or 119); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3833 (e.g., SEQ ID NO: 114 or 120); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3833 (e.g., SEQ ID NO: 115), and
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3833 (e.g., SEQ ID NO: 116); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3833 (e.g., SEQ ID NO: 117); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3833 (e.g., SEQ ID NO: 118).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3833 (e.g., SEQ ID NO: 121); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3833 (e.g., SEQ ID NO: 122).

In an embodiment, the antibody molecule comprises a VH encoded by the nucleotide sequence of SEQ ID NO: 177 (or a nucleotide sequence substantially identical thereto) or a VL encoded by the nucleotide sequence of SEQ ID NO: 178 (or a nucleotide sequence substantially identical thereto), or both.

In an embodiment, the antibody molecule is monoclonal antibody 3833. In an embodiment, monoclonal antibody 3833 is a humanized monoclonal antibody 3833. In an embodiment, the antibody molecule comprises a VH comprising the amino acid sequence of any of SEQ ID NO: 246-250, a VL comprising the amino acid sequence of any of SEQ ID NO: 251-253, or both.

In an embodiment, the antibody molecule binds, or substantially binds, to human APRIL, mouse APRIL, or both.

In an embodiment, the antibody molecule binds, or substantially binds, to human APRIL at an EC₅₀ of 20 nM or less, e.g., 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 20 nM, e.g., between 0.01 nM and 20 nM, between 0.1 nM and 20 nM, between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein. In an embodiment, the antibody molecule binds to human APRIL at an EC₅₀ of 5 nM or less, e.g., about 2.5 nM.

In an embodiment, the antibody molecule binds, or substantially binds, to mouse APRIL at an EC₅₀ of 100 nM or less, e.g., 80 nM or less, 60 nM or less, 40 nM or less, 20 nM or less, 10 nM or less, 9 nM or less 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 100 nM, e.g., between 0.001 nM and 50 nM, between 0.01 nM and 20 nM, between 0.1 nM and 10 nM, between 0.5 nM and 5 nM or between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein.

In an embodiment, the antibody molecule inhibits, or substantially inhibits, binding of APRIL (e.g., human APRIL) to TACI (e.g., human TACI), BCMA (e.g., human BCMA), or both. In an embodiment, the antibody molecule inhibits, or substantially inhibits, binding of APRIL (e.g., human APRIL, mouse APRIL, or both) to TACI (e.g., human TACI, mouse TACI, or both), at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human TACI at an IC₅₀ of 5 nM or less, e.g., about 4.05 nM.

In an embodiment, the antibody molecule inhibits, or substantially inhibits, binding of APRIL (e.g., human APRIL, mouse APRIL, or both) to BCMA (e.g., human BCMA, mouse BCMA, or both), at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human BCMA at an IC₅₀ of 1 nM or less, e.g., about 0.85 nM.

In an embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3631 (e.g., SEQ ID NO: 123 or 129); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3631 (e.g., SEQ ID NO: 124 or 130); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3631 (e.g., SEQ ID NO: 125), and
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3631 (e.g., SEQ ID NO: 126); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3631 (e.g., SEQ ID NO: 127); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3631 (e.g., SEQ ID NO: 128).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3631 (e.g., SEQ ID NO: 131); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3631 (e.g., SEQ ID NO: 132).

In an embodiment, the antibody molecule comprises a VH encoded by the nucleotide sequence of SEQ ID NO: 179 (or a nucleotide sequence substantially identical thereto) or a VL encoded by the nucleotide sequence of SEQ ID NO: 180 (or a nucleotide sequence substantially identical thereto), or both.

In an embodiment, the antibody molecule is monoclonal antibody 3631. In an embodiment, the antibody molecule is a humanized monoclonal antibody 3631.

In an embodiment, the antibody molecule binds, or substantially binds, to human APRIL, mouse APRIL, or both.

In an embodiment, the antibody molecule binds, or substantially binds, to human APRIL at an EC₅₀ of 20 nM or less, e.g., 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 20 nM, e.g., between 0.01 nM and 20 nM, between 0.1 nM and 20 nM, between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein. In an embodiment, the antibody molecule binds to human APRIL at an EC₅₀ of 5 nM or less, e.g., about 2.5 nM.

In an embodiment, the antibody molecule binds, or substantially binds, to mouse APRIL at an EC₅₀ of 100 nM or less, e.g., 80 nM or less, 60 nM or less, 40 nM or less, 20 nM or less, 10 nM or less, 9 nM or less 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 100 nM, e.g., between 0.001 nM and 50 nM, between 0.01 nM and 20 nM, between 0.1 nM and 10 nM, between 0.5 nM and 5 nM or between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein.

In an embodiment, the antibody molecule inhibits, or substantially inhibits, binding of APRIL (e.g., human APRIL) to TACI (e.g., human TACI), BCMA (e.g., human BCMA), or both. In an embodiment, the antibody molecule inhibits, or substantially inhibits, binding of APRIL (e.g., human APRIL, mouse APRIL, or both) to TACI (e.g., human TACI, mouse TACI, or both), at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human TACI at an IC₅₀ of 5 nM or less, e.g., about 4.05 nM.

In an embodiment, the antibody molecule inhibits, or substantially inhibits, binding of APRIL (e.g., human APRIL, mouse APRIL, or both) to BCMA (e.g., human BCMA, mouse BCMA, or both), at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human BCMA at an IC₅₀ of 1 nM or less, e.g., about 0.85 nM.

In an embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3732 (e.g., SEQ ID NO: 133 or 138); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3732 (e.g., SEQ ID NO: 134 or 139); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3732 (e.g., SEQ ID NO: 135), and
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3732 (e.g., SEQ ID NO: 136); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3732 (e.g., SEQ ID NO: 127); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3732 (e.g., SEQ ID NO: 137).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3732 (e.g., SEQ ID NO: 140); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3732 (e.g., SEQ ID NO: 141).

In an embodiment, the antibody molecule comprises a VH encoded by the nucleotide sequence of SEQ ID NO: 181 (or a nucleotide sequence substantially identical thereto) or a VL encoded by the nucleotide sequence of SEQ ID NO: 182 (or a nucleotide sequence substantially identical thereto), or both.

In an embodiment, the antibody molecule is monoclonal antibody 3732. In an embodiment, the antibody molecule is a humanized monoclonal antibody 3732.

In an embodiment, the antibody molecule binds, or substantially binds, to human APRIL, mouse APRIL, or both.

In an embodiment, the antibody molecule binds, or substantially binds, to human APRIL at an EC₅₀ of 20 nM or less, e.g., 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 20 nM, e.g., between 0.01 nM and 20 nM, between 0.1 nM and 20 nM, between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein. In an embodiment, the antibody molecule binds to human APRIL at an EC₅₀ of 5 nM or less, e.g., about 2.5 nM.

In an embodiment, the antibody molecule binds, or substantially binds, to mouse APRIL at an EC₅₀ of 100 nM or less, e.g., 80 nM or less, 60 nM or less, 40 nM or less, 20 nM or less, 10 nM or less, 9 nM or less 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 100 nM, e.g., between 0.001 nM and 50 nM, between 0.01 nM and 20 nM, between 0.1 nM and 10 nM, between 0.5 nM and 5 nM or between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein.

In an embodiment, the antibody molecule inhibits, or substantially inhibits, binding of APRIL (e.g., human APRIL) to TACI (e.g., human TACI), BCMA (e.g., human BCMA), or both.

In an embodiment, the antibody molecule inhibits, or substantially inhibits, binding of APRIL (e.g., human APRIL, mouse APRIL, or both) to TACI (e.g., human TACI, mouse TACI, or both), at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human TACI at an IC₅₀ of 5 nM or less, e.g., about 4.05 nM.

In an embodiment, the antibody molecule inhibits, or substantially inhibits, binding of APRIL (e.g., human APRIL, mouse APRIL, or both) to BCMA (e.g., human BCMA, mouse BCMA, or both), at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human BCMA at an IC₅₀ of 1 nM or less, e.g., about 0.85 nM.

In an embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4540 (e.g., SEQ ID NO: 154 or 159); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4540 (e.g., SEQ ID NO: 155 or 160); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4540 (e.g., SEQ ID NO: 156), and
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4540 (e.g., SEQ ID NO: 116); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4540 (e.g., SEQ ID NO: 157); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4540 (e.g., SEQ ID NO: 158).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 4540 (e.g., SEQ ID NO: 161); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 4540 (e.g., SEQ ID NO: 162).

In an embodiment, the antibody molecule comprises a VH encoded by the nucleotide sequence of SEQ ID NO: 186 (or a nucleotide sequence substantially identical thereto) or a VL encoded by the nucleotide sequence of SEQ ID NO: 187 (or a nucleotide sequence substantially identical thereto), or both.

In an embodiment, the antibody molecule is monoclonal antibody 4540. In an embodiment, monoclonal antibody 4540 is a humanized monoclonal antibody 4540. In an embodiment, the antibody molecule comprises a VH comprising the amino acid sequence of any of SEQ ID NO: 254-258, a VL comprising the amino acid sequence of any of SEQ ID NO: 259-261, or both.

In an embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4540-063 (e.g., SEQ ID NO: 154 or 276); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4540-063 (e.g., SEQ ID NO: 155 or 277); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4540-063 (e.g., SEQ ID NO: 156), and
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4540-063 (e.g., SEQ ID NO: 274); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4540-063 (e.g., SEQ ID NO: 275); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4540-063 (e.g., SEQ ID NO: 158).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 4540-063 (e.g., SEQ ID NO: 258); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 4540-063 (e.g., SEQ ID NO: 261).

In an embodiment, the antibody molecule comprises a VH encoded by the nucleotide sequence of SEQ ID NO: 301 (or a nucleotide sequence substantially identical thereto) or a VL encoded by the nucleotide sequence of SEQ ID NO: 302 (or a nucleotide sequence substantially identical thereto), or both.

In an embodiment, the antibody molecule is monoclonal antibody 4540-063.

In an embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4540-033 (e.g., SEQ ID NO: 154 or 159); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4540-033 (e.g., SEQ ID NO: 155 or 278); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4540-033 (e.g., SEQ ID NO: 156), and
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4540-033 (e.g., SEQ ID NO: 274); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4540-033 (e.g., SEQ ID NO: 275); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4540-033 (e.g., SEQ ID NO: 158).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 4540-033 (e.g., SEQ ID NO: 256); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 4540-033 (e.g., SEQ ID NO: 261).

In an embodiment, the antibody molecule comprises a VH encoded by the nucleotide sequence of SEQ ID NO: 303 (or a nucleotide sequence substantially identical thereto) or a VL encoded by the nucleotide sequence of SEQ ID NO: 302 (or a nucleotide sequence substantially identical thereto), or both.

In an embodiment, the antibody molecule is monoclonal antibody 4540-033.

In an embodiment, the antibody molecule comprises:
(i) a heavy chain variable region (VH), wherein the heavy chain variable region comprises three heavy chain complementarity determining regions (HCDR1, HCDR2, and HCDR3), wherein the VH comprises one, two, or all of the following: an HCDR1 comprising the amino acid sequence of D-Y-Y-X4-N(SEQ ID NO: 343), where X4 is I or M; an HCDR2 comprising the amino acid sequence of W-I-F-P-G-S-G-S-T-Y-Y-X12-X13-K-X15-X16-G, where X12 is N or A, X13 is E or Q, X15 is F or L, and X16 is K or Q (SEQ ID NO: 344); or an HCDR3 comprising the amino acid sequence of G-D-S-G-R-A-M-D-Y (SEQ ID NO: 156), and
(ii) a light chain variable region (VL), wherein the light chain variable region comprises three light chain complementarity determining regions (LCDR1, LCDR2, and LCDR3), wherein the VL comprises one, two, or all of the following: an LCDR1 comprising the amino acid sequence of X1-A-S-Q-D-I-N-K-Y-I-A, wherein X1 is K or Q (SEQ ID NO: 345); an LCDR2 comprising the amino acid sequence of Y-T-S-T-L-X6-X7, wherein X₆ is Q or E, and X7 is S or T (SEQ ID NO: 346); or an LCDR3 comprising the amino acid sequence of L-Q-Y-D-N-L-L-T (SEQ ID NO: 158).

In another embodiment, the antibody molecule comprises:
(i) a heavy chain variable region (VH), wherein the heavy chain variable region comprises three heavy chain complementarity determining regions (HCDR1, HCDR2, and HCDR3), wherein the VH comprises one, two, or all of the following: an HCDR1 comprising the amino acid sequence of G-Y-T-F-A-D-Y (SEQ ID NO: 154); an HCDR2 comprising the amino acid sequence of F-P-G-S-G-S(SEQ ID NO: 155); or an HCDR3 comprising the amino acid sequence of G-D-S-G-R-A-M-D-Y (SEQ ID NO: 156), and
(ii) a light chain variable region (VL), wherein the light chain variable region comprises three light chain complementarity determining regions (LCDR1, LCDR2, and LCDR3), wherein the VL comprises one, two, or all of the following: an LCDR1 comprising the amino acid sequence of X1-A-S-Q-D-I-N-K-Y-I-A, wherein X1 is K or Q (SEQ ID NO: 345); an LCDR2 comprising the amino acid sequence of Y-T-S-T-L-X6-X7 (SEQ ID NO: 346), wherein X6 is Q or E, and X7 is S or T; or an LCDR3 comprising the amino acid sequence of L-Q-Y-D-N-L-L-T (SEQ ID NO: 158).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of SEQ ID NOS: 161, 256 or 258; or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of SEQ ID NO: 162 or 261.

In an embodiment, the antibody molecule is monoclonal antibody 4540. In another embodiment, the antibody molecule is monoclonal antibody 4540-063. In yet another embodiment, the antibody molecule is monoclonal antibody 4540-033.

In an embodiment, the antibody molecule binds, or substantially binds, to human APRIL, mouse APRIL, or both.

In an embodiment, the antibody molecule binds, or substantially binds, to human APRIL at an EC₅₀ of 20 nM or less, e.g., 10 nM or less, 9 nM or less or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, or 0.1 nM or less, e.g., between 0.1 nM and 20 nM, e.g., between 0.1 nM and 10 nM, between 0.5 nM and 5 nM, or between 1 nM and 5 nM, e.g., as determined by a method described herein. In an embodiment, the antibody molecule binds to human APRIL at an EC₅₀ of 5 nM or less, e.g., about 2.5 nM.

In an embodiment, the antibody molecule binds, or substantially binds, to mouse APRIL at an EC₅₀ of 100 nM or less, e.g., 80 nM or less, 60 nM or less, 40 nM or less, 20 nM or less, 10 nM or less, 9 nM or less or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, or 0.1 nM or less, e.g., between 0.1 nM and 20 nM, e.g., between 0.1 nM and 10 nM, between 0.5 nM and 5 nM, or between 1 nM and 5 nM, e.g., as determined by a method described herein.

In an embodiment, the antibody molecule inhibits, or substantially inhibits, binding of APRIL (e.g., human APRIL) to TACI (e.g., human TACI), BCMA (e.g., human BCMA), or both.

In an embodiment, the antibody molecule inhibits, or substantially inhibits, binding of APRIL (e.g., human APRIL, mouse APRIL, or both) to TACI (e.g., human TACI, mouse TACI, or both), at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human TACI at an IC₅₀ of 5 nM or less, e.g., about 4.05 nM.

In an embodiment, the antibody molecule inhibits, or substantially inhibits, binding of APRIL (e.g., human APRIL, mouse APRIL, or both) to BCMA (e.g., human BCMA, mouse BCMA, or both), at an IC₅₀ of 200 nM or less, 150 nM or less, 100 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human BCMA at an IC₅₀ of 1 nM or less, e.g., about 0.85 nM.

In an embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 2621 (e.g., SEQ ID NO: 21 or 27); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 2621 (e.g., SEQ ID NO: 22 or 28); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 2621 (e.g., SEQ ID NO: 23), and
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 2621 (e.g., SEQ ID NO: 24); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 2621 (e.g., SEQ ID NO: 25); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 2621 (e.g., SEQ ID NO: 26).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 2621 (e.g., SEQ ID NO: 29); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 2621 (e.g., SEQ ID NO: 30).

In an embodiment, the antibody molecule comprises a VH encoded by the nucleotide sequence of SEQ ID NO: 75 (or a nucleotide sequence substantially identical thereto) or a VL encoded by the nucleotide sequence of SEQ ID NO: 76 (or a nucleotide sequence substantially identical thereto), or both.

In an embodiment, the antibody molecule is monoclonal antibody 2621. In an embodiment, the antibody molecule is a humanized monoclonal antibody 2621.

In an embodiment, the antibody molecule binds, or substantially binds, to human APRIL.

In an embodiment, the antibody molecule binds, or substantially binds, to human APRIL at an EC₅₀ of 20 nM or less, e.g., 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 20 nM, e.g., between 0.01 nM and 20 nM, between 0.1 nM and 20 nM, between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein. In an embodiment, the antibody molecule binds to human APRIL at an EC₅₀ of 1 nM or less, e.g., about 0.7 nM. In an embodiment, the antibody molecule does not bind, or binds to mouse APRIL with low affinity, e.g., at an EC₅₀ of 1000 nM or more, e.g., 2000 nM or more, e.g., as determined by a method described herein.

In an embodiment, the antibody molecule inhibits, or substantially inhibits, binding of APRIL (e.g., human APRIL) to TACI (e.g., human TACI). In an embodiment, the antibody molecule inhibits, or substantially inhibits, binding of APRIL (e.g., human APRIL) to TACI (e.g., human TACI), at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human TACI at an IC₅₀ of about 1 nM or less.

In an embodiment, the antibody molecule does not inhibit, or does not substantially inhibit, binding of APRIL (e.g., human APRIL) to BCMA (e.g., human BCMA).

In an embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 3125 (e.g., SEQ ID NO: 11 or 47); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 3125 (e.g., SEQ ID NO: 42 or 48); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 3125 (e.g., SEQ ID NO: 43), and
(ii) a light chain variable region (VH), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 3125 (e.g., SEQ ID NO: 44); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 3125 (e.g., SEQ ID NO: 45); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 3125 (e.g., SEQ ID NO: 46).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 3125 (e.g., SEQ ID NO: 49); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 3125 (e.g., SEQ ID NO: 50).

In an embodiment, the antibody molecule comprises a VH encoded by the nucleotide sequence of SEQ ID NO: 79 (or a nucleotide sequence substantially identical thereto) or a VL encoded by the nucleotide sequence of SEQ ID NO: 80 (or a nucleotide sequence substantially identical thereto), or both.

In an embodiment, the antibody molecule is monoclonal antibody 3125. In an embodiment, the antibody molecule is a humanized monoclonal antibody 3125.

In an embodiment, the antibody molecule binds, or substantially binds, to human APRIL. In an embodiment, the antibody molecule binds, or substantially binds, to human APRIL at an EC₅₀ of 20 nM or less, e.g., 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 20 nM, e.g., between 0.01 nM and 20 nM, between 0.1 nM and 20 nM, between 0.1 nM and 10 nM, between 0.5 nM and 5 nM, between 1 nM and 5 nM, between 0.001 nM and 0.1 nM, between 0.001 nM and 0.01 nM, between 0.001 nM and 0.005 nM, between 0.01 nM and 0.05 nM, or between 0.01 nM and 0.1 nM, e.g., as determined by a method described herein. In an embodiment, the antibody molecule binds to human APRIL at an EC₅₀ of 20 nM or less, e.g., about 13 nM. In an embodiment, the antibody molecule does not bind, or binds to mouse APRIL with low affinity, e.g., at an EC₅₀ of 1000 nM or more, e.g., 2000 nM or more, e.g., as determined by a method described herein.

In an embodiment, the antibody molecule inhibits, or substantially inhibits, binding of APRIL (e.g., human APRIL) to TACI (e.g., human TACI). In an embodiment, the antibody molecule inhibits, or substantially inhibits, binding of APRIL (e.g., human APRIL) to TACI (e.g., human TACI), at an IC₅₀ of 50 nM or less, e.g., 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.8 nM or less, 0.6 nM or less, 0.4 nM or less, 0.2 nM or less, 0.1 nM or less, 0.05 nM or less, 0.02 nM or less, or 0.01 nM or less, e.g., between 0.01 nM and 50 nM, between 0.1 nM and 50 nM, between 0.1 nM and 25 nM, between 0.1 nM and 10 nM, between 0.1 nM and 5 nM, between 0.1 nM and 1 nM, between 0.1 nM and 0.5 nM, between 0.5 nM and 5 nM, or between 1 nM and 5 nM, e.g., as determined by a method described herein. In an embodiment, the antibody molecule inhibits binding of human APRIL to human TACI at an IC₅₀ of 150 nM or less, e.g., about 112.97 nM. In an embodiment, the antibody molecule does not inhibit, or does not substantially inhibit, binding of APRIL (e.g., human APRIL) to BCMA (e.g., human BCMA).

In an embodiment, the antibody molecule comprises:
(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody 4439 (e.g., SEQ ID NO: 266 or 269); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody 4439 (e.g., SEQ ID NO: 267 or 270); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody 4439 (e.g., SEQ ID NO: 268), and
(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody 4439 (e.g., SEQ ID NO: 146); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody 4439 (e.g., SEQ ID NO: 147); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody 4439 (e.g., SEQ ID NO: 148).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody 4439 (e.g., SEQ ID NO: 271); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody 4439 (e.g., SEQ ID NO: 272).

In an embodiment, the antibody molecule comprises a VH encoded by the nucleotide sequence of SEQ ID NO: 297 (or a nucleotide sequence substantially identical thereto) or a VL encoded by the nucleotide sequence of SEQ ID NO: 298 (or a nucleotide sequence substantially identical thereto), or both.

In an embodiment, the antibody molecule is monoclonal antibody 4439. In an embodiment, monoclonal antibody 4439 is humanized monoclonal antibody 4439.

In an embodiment, the antibody molecule is an IgG antibody molecule, e.g., comprising a heavy chain constant region of IgG, e.g., chosen from IgG1, IgG2 (e.g., IgG2a), IgG3, or IgG4, e.g., IgG2 or IgG4. In an embodiment, the antibody molecule is an IgG1 antibody molecule. In another embodiment, the antibody molecule is an IgG2 antibody molecule. In an embodiment, the antibody molecule comprises a light chain constant region of kappa or lambda light chain.

In an embodiment, the antibody molecule comprises an Fc region. In an embodiment, the Fc region comprises one or more mutations located at the interface between the CH2 and CH3 domains (e.g., to increase the binding affinity to neonatal receptor FcRn and/or the half-life of the antibody molecule).

In an embodiment, the Fc region comprises one or more mutations, e.g., one or more (e.g., 2, 3, 4, 6 or all) mutations chosen from T250Q, M252Y, S254T, T256E, M428L, H433K, N434F, or any combination thereof, of IgG1. In an embodiment, the Fc region comprises one or more mutations at positions 233-236 or 322 of human IgG1 or IgG2, or one or more substitutions at positions 327, 330 or 331 of human IgG4 (e.g., to reduce complement-dependent cytotoxicity (CDC)). In an embodiment, the Fc region comprises one or more (e.g., 2, 3, 4, 6 7 or all) mutations chosen from E233P, L234V, L235A, G236, K322A, A327G, A330S, P331S, or any combination thereof.

In an embodiment, the antibody molecule is a humanized antibody molecule, e.g., as described in Table 5, e.g., comprising one or more framework regions derived from human framework germline sequence.

In an embodiment, the antibody molecule comprises two heavy chain variable regions and two light chain variable regions. In an embodiment, the antibody molecule is a Fab, F(ab′)2, Fv, Fd, or a single chain Fv fragment (scFv).

Animal Models The antibody molecules described herein can be evaluated in vivo, e.g., using various animal models. For example, an animal model can be used to test the efficacy of an antibody molecule described herein in inhibiting APRIL and/or in treating or preventing a disorder described herein, e.g., IgA nephropathy. Animal models can also be used, e.g., to investigate for side effects, measure concentrations of antibody molecules in situ, demonstrate correlations between an APRIL function and a disorder described herein (e.g., IgA nephropathy).

Exemplary animal models for IgA nephropathy that can be used for evaluating an antibody molecule described herein include, but are not limited to, a ddY mouse model for spontaneous IgA nephritis (Imai et al. Kidney Int. 1985; 27(5):756-761); a mouse model utilizing inert proteins or a common viral pathogen as the inciting antigen (Emancipator et al. Curr. Protoc. Immunol. 2001 May; Chapter 15: Unit 15.11), a rat model by noninfectious protein antigens (Emancipator et al. Curr. Protoc.

Immunol. 2001 May; Chapter 15: Unit 15.11); a chronic mouse model of IgA immune-complex-associated nephropathy (Montinaro et al. Nephrol. Dial. Transplant. 1995; 10(11): 2035-2042); the Gne M712T mouse as a model for human glomerulopathy (Kakani et al. Am. J. Pathol. 2012; 180(4):1431-1440); a mouse IgA nephropathy model with the MBP-20-peptide fusion protein (Zhang et al. Anat. Rec. (Hoboken). 2010; 293(10): 1729-1737); and a mouse model for IgA immune complex nephritis (Rifai et al. J Exp Med. 1979; 150(5):1161-1173). Other animal models for IgA nephropathy are described, e.g., in Tomino et al. J. Nephrol. 2008; 21(4):463-467; Endo Ren. Fail. 1997; 19(3):347-371; and Rifai Kidney Int. 1987; 31(1):1-7.

Exemplary animal models for other disorders described herein are also known in the art. Exemplary types of animals that can be used to evaluate the antibody molecules described herein include, but are not limited to, mice, rats, rabbits, guinea pigs, and monkeys.

### Pharmaceutical Compositions and Kits

In some aspects, this disclosure provides compositions, e.g., pharmaceutically acceptable compositions, which include an antibody molecule described herein (e.g., a humanized antibody molecule described herein), formulated together with a pharmaceutically acceptable carrier.

As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g., by injection or infusion). In certain embodiments, less than about 5%, e.g., less than about 4%, 3%, 2%, or 1% of the antibody molecules in the pharmaceutical composition are present as aggregates. In other embodiments, at least about 95%, e.g., at least about 96%, 97%, 98%, 98.5%, 99%, 99.5%, 99.8%, or more of the antibody molecules in the pharmaceutical composition are present as monomers. In some embodiments, the level of aggregates or monomers is determined by chromatography, e.g., high performance size exclusion chromatography (HP-SEC).

The compositions set out herein may be in a variety of forms. These include, for example, liquid, semi-solid and solid dosage forms, such as liquid solutions (e.g., injectable and infusible solutions), dispersions or suspensions, liposomes, and suppositories. A suitable form depends on the intended mode of administration and therapeutic application. Typical suitable compositions are in the form of injectable or infusible solutions. One suitable mode of administration is parenteral (e.g., intravenous, subcutaneous, intraperitoneal, intramuscular). In some embodiments, the antibody molecule is administered by intravenous infusion or injection. In certain embodiments, the antibody is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibody molecules described herein can be administered by a variety of methods. Several are known in the art, and for many therapeutic, prophylactic, or diagnostic applications, an appropriate route/mode of administration is intravenous injection or infusion. For example, the antibody molecules can be administered by intravenous infusion at a rate of less than 10 mg/min; preferably less than or equal to 5 mg/min to reach a dose of about 1 to 100 mg/m², preferably about 5 to 50 mg/m², about 7 to 25 mg/m² and more preferably, about 10 mg/m². As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody molecule can be orally administered, for example, with an inert diluent or an assimilable edible carrier. The antibody molecule (and other ingredients, if desired) may also be enclosed in a hard or soft shell gelatin capsule, compressed into tablets, or incorporated directly into the subject's diet. For oral therapeutic administration, the antibody molecule may be incorporated with excipients and used in the form of ingestible tablets, buccal tablets, troches, capsules, elixirs, suspensions, syrups, wafers, and the like. To administer an antibody molecule by other than parenteral administration, it may be necessary to coat the compound with, or co-administer the compound with, a material to prevent its inactivation. Therapeutic, prophylactic, or diagnostic compositions can also be administered with medical devices, and several are known in the art.

Dosage regimens are adjusted to provide the desired response (e.g., a therapeutic, prophylactic, or diagnostic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the antibody molecule and the particular therapeutic, prophylactic, or diagnostic effect to be achieved, and (b) the limitations inherent in the art of compounding such an antibody molecule for the treatment of sensitivity in individuals.

An exemplary, non-limiting range for a therapeutically, prophylactically, or diagnostically effective amount of an antibody molecule is about 0.1-50 mg/kg body weight of a subject, e.g., about 0.1-30 mg/kg, e.g., about 1-30, 1-15, 1-10, 1-5, 5-10, or 1-3 mg/kg, e.g., about 1, 2, 3, 4, 5, 6, 7, 8, 9, 10, 15, 20, 30, 40, or 50 mg/kg. In some embodiments, the antibody molecule is administered in a dose of about 2 mg/kg. In some embodiments, the antibody molecule is administered in a dose of about 4 mg/kg. In some embodiments, the antibody molecule is administered in a dose of about 8 mg/kg. In some embodiments, the antibody molecule is administered in a dose between 2-4 or 4-8 mg/kg. The antibody molecule can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², e.g., about 10 mg/m². It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed compositions.

The pharmaceutical compositions herein may include a “therapeutically effective amount,” “prophylactically effective amount,” or “diagnostically effectively amount” of an antibody molecule described herein.

A “therapeutically effective amount” refers to an amount effective, at dosages and for periods of time necessary, to achieve the desired therapeutic result. A therapeutically effective amount of the antibody molecule may vary according to factors such as the disease state, age, sex, and weight of the individual, and the ability of the antibody or antibody portion to elicit a desired response in the individual.

A therapeutically effective amount is also one in which any toxic or detrimental effect of the antibody molecule is outweighed by the therapeutically beneficial effects. A “therapeutically effective dosage” typically inhibits a measurable parameter by at least about 20%, e.g., by at least about 40%, by at least about 60%, or by at least about 80% relative to untreated subjects. The measurable parameter may be, e.g., hematuria, colored urine, foamy urine, pain, swelling (edema) in the hands and feet, or high blood pressure. The ability of an antibody molecule to inhibit a measurable parameter can be evaluated in an animal model system predictive of efficacy in treating or preventing IgA nephropathy. Alternatively, this property of a composition can be evaluated by examining the ability of the antibody molecule to inhibit APRIL, e.g., by an in vitro assay.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

A “diagnostically effective amount” refers to an amount effective, at dosages and for periods of time necessary, to achieve the desired diagnostic result. Typically, a diagnostically effective amount is one in which a disorder, e.g., a disorder described herein, e.g., IgA nephropathy, can be diagnosed in vitro, ex vivo, or in vivo.

Also within this disclosure is a kit that comprises an antibody molecule, described herein. The kit can include one or more other elements including: instructions for use; other reagents, e.g., a label, a therapeutic agent, or an agent useful for chelating, or otherwise coupling, an antibody molecule to a label or therapeutic agent, or a radioprotective composition; devices or other materials for preparing the antibody molecule for administration; pharmaceutically acceptable carriers; and devices or other materials for administration to a subject.

Nucleic Acids The present disclosure also features nucleic acids comprising nucleotide sequences that encode the antibody molecules (e.g., heavy and light chain variable regions and CDRs of the antibody molecules), as described herein.

For example, the present disclosure features a first and second nucleic acid encoding heavy and light chain variable regions, respectively, of an antibody molecule chosen from one or more of the antibody molecules disclosed herein, e.g., an antibody molecule of Table 1 or 5, or a portion of an antibody molecule, e.g., the variable regions of Table 2. The nucleic acid can comprise a nucleotide sequence encoding any one of the amino acid sequences in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein).

In certain embodiments, the nucleic acid can comprise a nucleotide sequence encoding at least one, two, or three CDRs from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In some embodiments, the nucleic acid can comprise a nucleotide sequence encoding at least one, two, or three CDRs from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In some embodiments, the nucleic acid can comprise a nucleotide sequence encoding at least one, two, three, four, five, or six CDRs from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding at least one, two, or three CDRs from a heavy chain variable region having the nucleotide sequence as set forth in Table 2, a sequence substantially homologous thereto (e.g., a sequence at least about 85%, 90%, 95%, 99% or more identical thereto, and/or capable of hybridizing under the stringency conditions described herein). In some embodiments, the nucleic acid can comprise a nucleotide sequence encoding at least one, two, or three CDRs from a light chain variable region having the nucleotide sequence as set forth in Table 2, or a sequence substantially homologous thereto (e.g., a sequence at least about 85%, 90%, 95%, 99% or more identical thereto, and/or capable of hybridizing under the stringency conditions described herein). In certain embodiments, the nucleic acid can comprise a nucleotide sequence encoding at least one, two, three, four, five, or six CDRs from heavy and light chain variable regions having the nucleotide sequence as set forth in Table 2, or a sequence substantially homologous thereto (e.g., a sequence at least about 85%, 90%, 95%, 99% or more identical thereto, and/or capable of hybridizing under the stringency conditions described herein).

In certain embodiments, the nucleic acid comprises a nucleotide sequence as set forth in Table 2 or a sequence substantially homologous thereto (e.g., a sequence at least about 85%, 90%, 95%, 99% or more identical thereto, and/or capable of hybridizing under the stringency conditions described herein).

In some embodiments, the nucleic acid comprises a portion of a nucleotide sequence as set forth in Table 2 or a sequence substantially homologous thereto (e.g., a sequence at least about 85%, 90%, 95%, 99% or more identical thereto, and/or capable of hybridizing under the stringency conditions described herein).

The portion may encode, for example, a variable region (e.g., VH or VL); one, two, or three or more CDRs; or one, two, three, or four or more framework regions.

The nucleic acids disclosed herein include deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

In some aspects, the application features host cells and vectors containing the nucleic acids described herein. The nucleic acids may be present in a single vector or separate vectors present in the same host cell or separate host cell, as described in more detail below.

### Vectors

Further provided herein are vectors that comprise nucleotide sequences encoding an antibody molecule described herein.

In an embodiment, the vector comprises a nucleotide encoding an antibody molecule described herein, e.g., as described in Table 1 or 5. In another embodiment, the vector comprises a nucleotide sequence described herein, e.g., in Table 2. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

### Cells

The present disclosure also provides cells (e.g., host cells) comprising a nucleic acid encoding an antibody molecule as described herein. For example, the host cells may comprise a nucleic acid molecule having a nucleotide sequence described in Table 2, a sequence substantially homologous thereto (e.g., a sequence at least about 85%, 90%, 95%, 99% or more identical thereto, and/or capable of hybridizing under the stringency conditions described herein), or a portion of one of said nucleic acids. Additionally, the host cells may comprise a nucleic acid molecule encoding an amino acid sequence of Table 1 or 5, a sequence substantially homologous thereto (e.g., a sequence at least about 80%, 85%, 90%, 95%, 99% or more identical thereto), or a portion of one of said sequences.

In some embodiments, the host cells are genetically engineered to comprise nucleic acids encoding the antibody molecule described herein.

In certain embodiments, the host cells are genetically engineered by using an expression cassette.

The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells. In an embodiment, the cell (e.g., host cell) is an isolated cell.

### Uses of Antibody Molecules

The antibody molecules disclosed herein, as well as the pharmaceutical compositions disclosed herein, have in vitro, ex vivo, and in vivo therapeutic, prophylactic, and/or diagnostic utilities.

In an embodiment, the antibody molecule reduces (e.g., inhibits, blocks, or neutralizes) one or more biological activities of APRIL. For example, these antibodies molecules can be administered to cells in culture, in vitro or ex vivo, or to a subject, e.g., a human subject, e.g., in vivo, to reduce (e.g., inhibits, blocks, or neutralizes) one or more biological activities of APRIL. In an embodiment, the antibody molecule inhibits, or substantially inhibit, binding of APRIL, e.g., human APRIL, to TACI, BCMA, or both. Accordingly, in an aspect, the disclosure provides a method of improving kidney function in a subject (e.g., a subject having or at risk of developing a disease, disorder, or condition as described herein), the method comprising administering to the subject an antibody molecule described herein. Improvement in kidney function can include, for example, reduction, reversal, or prevention of progression of decreased kidney function. Improvement in kidney function can, in some instances, include kidney regeneration in the subject. In some embodiments, improvement in kidney function includes increasing eGFR and/or reducing proteinuria in a kidney of the subject. In an aspect, the disclosure provides a method of treating, preventing, or diagnosing a disorder, e.g., a disorder described herein (e.g., IgA nephropathy (IgAN) or disorders associated with IgAN (e.g., an advanced chronic kidney disease (CKD), post-transplant IgAN, pediatric IgAN, Henoch-Schonlein purpura (HSP) or cutaneous vasculitis, IgAN with crescentic glomerulonephritis (GN)), IgA vasculitis, IgA dermatitis (e.g., IgA dermatitis herpetiformis, IgA bullous dermatosis), IgM mediated neuropathy (e.g., anti-MAG peripheral neuropathy or IgM mediated neuropathy associated with anti-GM1 antibodies), Waldenstrom's Macroglobulinemia (WM), or lupus nephritis), in a subject, the method comprising administering to the subject an antibody molecule described herein, such that the disorder is treated, prevented, or diagnosed.

For example, the disclosure provides a method comprising contacting the antibody molecule described herein with cells in culture, e.g. in vitro or ex vivo, or administering the antibody molecule described herein to a subject, e.g., in vivo, to treat, prevent, or diagnose a disorder, e.g., a disorder associated with APRIL (e.g., IgA nephropathy (IgAN) or a disorder associated with IgAN (e.g., an advanced chronic kidney disease (CKD), post-transplant IgAN, pediatric IgAN, Henoch-Schonlein purpura (HSP) or cutaneous vasculitis, IgAN with crescentic glomerulonephritis (GN)), IgA vasculitis, IgA dermatitis (e.g., IgA dermatitis herpetiformis, IgA bullous dermatosis), IgM mediated neuropathy (e.g., anti-MAG peripheral neuropathy or IgM mediated neuropathy associated with anti-GM1 antibodies), Waldenstrom's Macroglobulinemia (WM), or lupus nephritis). In some embodiments, a disease, disorder, or condition treatable or preventable with an antibody molecule as described herein is a disease, disorder, or condition associated with decreased kidney function. In some embodiments, a disease, disorder, or condition treatable or preventable with an antibody molecule as described herein is a disease, disorder, or condition associated with risk of kidney failure. In some embodiments, a disease, disorder, or condition treatable or preventable with an antibody molecule as described herein is an autoimmune disorder (e.g., an autoantibody-related disorder, e.g., an IgM autoantibody-related disorder).

As used herein, the term “subject” is intended to include human and non-human animals. In some embodiments, the subject is a human subject, e.g., a human patient having a disorder described herein (e.g., IgA nephropathy (IgAN) or a disorder associated with IgAN (e.g., an advanced chronic kidney disease (CKD), post-transplant IgAN, pediatric IgAN, Henoch-Schonlein purpura (HSP) or cutaneous vasculitis, IgAN with crescentic glomerulonephritis (GN))), or at risk of having a disorder described herein (e.g., IgA nephropathy (IgAN) or disorders associated with IgAN (e.g., an advanced chronic kidney disease (CKD), post-transplant IgAN, pediatric IgAN, Henoch-Schonlein purpura (HSP) or cutaneous vasculitis, IgAN with crescentic glomerulonephritis (GN)), IgA vasculitis, IgA dermatitis (e.g., IgA dermatitis herpetiformis, IgA bullous dermatosis), IgM mediated neuropathy (e.g., anti-MAG peripheral neuropathy or IgM mediated neuropathy associated with anti-GM1 antibodies), Waldenstrom's Macroglobulinemia (WM), or lupus nephritis). In some embodiments, the subject is a human subject in need of improved kidney function (e.g, reduction or reversal of decreased kidney function and/or kidney regeneration). The term “non-human animals” includes mammals and non-mammals, such as non-human primates. In some embodiments, the subject is a human. The methods and compositions described herein are suitable for treating human patients a disorder described herein (e.g., IgA nephropathy (IgAN) or a disorder associated with IgAN (e.g., an advanced chronic kidney disease (CKD), post-transplant IgAN, pediatric IgAN, Henoch-Schonlein purpura (HSP) or cutaneous vasculitis, IgAN with crescentic glomerulonephritis (GN)), IgA vasculitis, IgA dermatitis (e.g., IgA dermatitis herpetiformis, IgA bullous dermatosis), IgM mediated neuropathy (e.g., anti-MAG peripheral neuropathy or IgM mediated neuropathy associated with anti-GM1 antibodies), Waldenstrom's Macroglobulinemia (WM), or lupus nephritis).

Patients having a disorder described herein (e.g., IgA nephropathy (IgAN) or a disorder associated with IgAN (e.g., an advanced chronic kidney disease (CKD), post-transplant IgAN, pediatric IgAN, Henoch-Schonlein purpura (HSP) or cutaneous vasculitis, IgAN with crescentic glomerulonephritis (GN)), IgA vasculitis, IgA dermatitis (e.g., IgA dermatitis herpetiformis, IgA bullous dermatosis), IgM mediated neuropathy (e.g., anti-MAG peripheral neuropathy or IgM mediated neuropathy associated with anti-GM1 antibodies), Waldenstrom's Macroglobulinemia (WM), or lupus nephritis) include those who have developed the disorder, but are (at least temporarily) asymptomatic, patients who have exhibited a symptom of the disorder, or patients having a disorder related to or associated with the disorder.

### Improving Kidney Function

The antibody molecules, pharmaceutical compositions, and methods described herein can be used to improve kidney function in a subject (e.g., a subject having or at risk of developing a disease, disorder, or condition as described herein). In some embodiments, improvement in kidney function comprises reduction, reversal, or prevention of progression of decreased kidney function. In some embodiments, improvement in kidney function includes kidney regeneration in the subject. In some embodiments, improvement in kidney function includes increasing eGFR and/or reducing proteinuria (e.g., as determined by measuring urine protein/creatinine ratio (uPCR)) in a kidney of the subject.

In some embodiments, the antibody molecule, pharmaceutical composition, or method improves kidney function (e.g., as determined by measuring eGFR and/or uPCR) in the subject within 1, 2, 3, 4, 5, 6,7, 8,9, 10, 11, 12, 13, 14, 15, 16, 17, 18, 19, 20, 21, 22, 23, 24, 25, 26, 27, 28, 29, 30, 31, 32, 33, 34, 35, or 36 months after administration (e.g., after first, second, or third administration) of the antibody molecule. In some embodiments, the antibody molecule, pharmaceutical composition, or method improves kidney function (e.g., as determined by measuring eGFR and/or uPCR) in the subject within 1-5, 5-10, 10-15, 15-20, 20-25, 25-30, 30-35, 35-40, 40-45, or 45-50 months after administration (e.g., after first, second, or third administration) of the antibody molecule. In some embodiments, the antibody molecule, pharmaceutical composition, or method improves kidney function (e.g., as determined by measuring eGFR and/or uPCR) in the subject after 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 administrations of the antibody molecule. In some embodiments, the antibody molecule, pharmaceutical composition, or method improves kidney function (e.g., as determined by measuring eGFR and/or uPCR) in the subject after between 1-5, 5-10, 10-15, 15-20, 20-25, 25-30, 30-35, 35-40, 40-45, or 45-50 administrations of the antibody molecule. In some embodiments, the antibody molecule, pharmaceutical composition, or method improves kidney function (e.g., as determined by measuring eGFR and/or uPCR) for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In some embodiments, the antibody molecule, pharmaceutical composition, or method improves kidney function (e.g., as determined by measuring eGFR and/or uPCR) in the subject for a period of at least 1-5, 5-10, 10-15, 15-20, 20-25, 25-30, 30-35, 35-40, 40-45, or 45-50 months. In some embodiments, the antibody molecule, pharmaceutical composition, or method improves kidney function (e.g., as determined by measuring eGFR and/or uPCR) in the subject after 1, 2, 3, 4, 5, 6, 7, 8,9, 10, 11, 12, 13, 14, 15, 16, 17, 18, 19, 20, 21, 22, 23, 24, 25, 26, 27, 28, 29, 30, 31, 32, 33, 34, 35, or 36 administrations of the antibody molecule.

In some embodiments, administration of an antibody molecule as described herein results in an increase in eGFR in the subject. In an embodiment, administration of the antibody molecule results in an increase in eGFR within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after the administration (e.g., after the first, second, or third administration). In an embodiment, administration of the antibody molecule results in an increase in eGFR of at least 10%, 20%, 30%, 40%, 50%, 60%, 70%, 75%, 80%, 85%, 90%, 95%, 96%, 97%, 98%, or 99%, e.g., within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after the administration (e.g., after the first, second, or third administration).

In some embodiments, administration of an antibody molecule as described herein results in a decrease in uPCR in the subject. In an embodiment, administration of the antibody molecule results in a decrease in uPCR within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after the administration (e.g., after the first, second, or third administration). In an embodiment, administration of the antibody molecule results in a decrease in uPCR of at least 5%, 10%, 20%, 30%, 40%, 50%, 60%, 70%, 75%, 80%, 85%, 90%, 95%, 96%, 97%, 98%, or 99%, e.g., within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after the administration (e.g., after the first, second, or third administration). In an embodiment, the decrease in uPCR is maintained for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months.

### Serum Immunoglobulin

In some embodiments, administration of an antibody molecule as described herein results in a decrease in serum IgA levels (e.g., compared to prior to the administration). In an embodiment, the decrease in serum IgA levels is maintained for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In an embodiment, the serum IgA levels are decreased by at least 40%, 50%, 60%, 70%, 75%, 80%, 85%, 90%, 95%, 96%, 97%, 98%, or 99%, e.g., within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after the administration (e.g., after the first, second, or third administration). In an embodiment, the serum IgA levels are decreased by at least 50, 100, 110, 120, 130, 140, 150, 160, 170, 180, 190, 200, 210, 220, 230, 240, or 250 mg/dL, e.g., within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after the administration (e.g., after the first, second, or third administration).

In some embodiments, administration of an antibody molecule as described herein results in a decrease in serum a-g-IgA levels (e.g., compared to prior to the administration). In an embodiment, the decrease in serum a-g-IgA levels is maintained for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In an embodiment, the serum a-g-IgA levels are decreased by at least 40%, 50%, 60%, 70%, 75%, 80%, 85%, 90%, 95%, 96%, 97%, 98%, or 99%, e.g., within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after the administration (e.g., after the first, second, or third administration). In certain embodiments, the a-g-IgA comprises a-g-IgA1.

In some embodiments, administration of an antibody molecule as described herein results in a decrease in serum IgG levels (e.g., compared to prior to the administration). In an embodiment, the decrease in serum IgG levels is maintained for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In an embodiment, the serum IgG levels are decreased by at least 40%, 50%, 60%, 70%, 75%, 80%, 85%, 90%, 95%, 96%, 97%, 98%, or 99%, e.g., within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after the administration (e.g., after the first, second, or third administration). In an embodiment, the serum IgG levels are decreased by at least 100, 150, 200, 250, 300, 350, 400, 450, 500, 550, 600, 650, 700, 750, or 800 mg/dL, e.g., within 1, 2, 3, 4, 5, 6, 7, 8,9, 10, 11, 12, 13, 14, 15, 16, 17, 18, 19, 20, 21, 22, 23, 24, 25, 26, 27, 28, 29, 30, 31, 32, 33, 34, 35, or 36 months after the administration (e.g., after the first, second, or third administration). In some embodiments, administration of an antibody molecule as described herein results in a decrease in serum IgM levels (e.g., compared to prior to the administration). In an embodiment, the decrease in serum IgM levels is maintained for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In an embodiment, the serum IgM levels are decreased by at least 40%, 50%, 60%, 70%, 75%, 80%, 85%, 90%, 95%, 96%, 97%, 98%, or 99%, e.g., within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after the administration (e.g., after the first, second, or third administration). In an embodiment, the serum IgM levels are decreased by at least 10, 20, 25, 30, 35, 40, 45, 50, 55, 60, 65, 70, 75, 80, 85, 90, or 95 mg/dL, e.g., within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after the administration (e.g., after the first, second, or third administration).

### Methods of Treating or Preventing Disorders

The antibody molecules, pharmaceutical compositions, and methods described herein can be used to treat or prevent disorders associated with APRIL or symptoms thereof in a subject in need thereof. In some embodiments, a disease, disorder, or condition treatable or preventable with an antibody molecule as described herein is a disease, disorder, or condition associated with decreased kidney function. In some embodiments, a disease, disorder, or condition treatable or preventable with an antibody molecule as described herein is a disease, disorder, or condition associated with risk of kidney failure. In certain embodiments, the subject has elevated proteinuria compared to a subject that does not have the disease, disorder, or condition (e.g., a healthy subject). In an embodiment, the subject has proteinuria greater than or equal to a uPCR of 0.75 g/g or urine protein 1 g/day prior to the administration. In an embodiment, the subject has proteinuria lower than 0.1, 0.2, 0.3, 0.4, 0.5, 0.6, or 0.7 g/day after the administration (e.g., within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months of the administration).Exemplary disorders or conditions that can be associated with APRIL include, but are not limited to IgA nephropathy (IgAN) or a disorder associated with IgAN (e.g., an advanced chronic kidney disease (CKD), post-transplant IgAN, pediatric IgAN, Henoch-Schonlein purpura (HSP) or cutaneous vasculitis, IgAN with crescentic glomerulonephritis (GN)), diabetic nephropathy, IgM mediated neuropathy (e.g., anti-MAG peripheral neuropathy or IgM mediated neuropathy associated with anti-GM1 antibodies), cancer (e.g., hematological cancer (e.g., B-cell non-Hodgkin's lymphoma, chronic lymphocytic leukemia, Hodgkin's lymphoma, multiple myeloma, Waldenstrom macroglobulinemia, and lymphoplasmacytic lymphoma) or solid tumors (e.g., colorectal cancer, breast cancer (e.g., breast carcinoma), esophageal cancer (e.g., esophageal adenocarcinoma), brain cancer (e.g., glioblastoma), and kidney cancer (e.g., renal cell carcinoma)), immunoproliferative disorders (e.g., monoclonal IgA hypergammaglobulinemia), vasculitis (e.g., kidney vasculitis, Henoch-Schonlein purpura (IgA associated vasculitis), and post-streptococcal glomerulonephritis), autoimmune disorders (e.g., rheumatoid arthritis, systemic lupus erythematosus, lupus nephritis, IgA dermatitis (e.g., IgA dermatitis herpetiformis, linear IgA bullous disease/linear immunoglobulin A (IgA) dermatosis, and IgA-mediated epidermolysis bullosa acquisita), IgA pemphigus, celiac disease, and alcoholic cirrhosis. In an embodiment, the disorder is associated with aberrant expression of IgA. In an embodiment, the antibody molecule is used to treat a subject having a disorder described herein, or is at risk of developing a disorder described herein.

In an embodiment, the disorder associated with APRIL is IgA nephropathy (IgAN) or a disorder associated with IgAN (e.g., an advanced chronic kidney disease (CKD), post-transplant IgAN, pediatric IgAN, Henoch-Schonlein purpura (HSP) or cutaneous vasculitis, IgAN with crescentic glomerulonephritis (GN)), IgA vasculitis, IgA dermatitis (e.g., IgA dermatitis herpetiformis, IgA bullous dermatosis), IgM mediated neuropathy (e.g., anti-MAG peripheral neuropathy or IgM mediated neuropathy associated with anti-GM1 antibodies), Waldenstrom's Macroglobulinemia (WM), or lupus nephritis. In an embodiment, the disorder is IgA nephropathy (IgAN). In an embodiment, the disorder is IgA nephropathy in a subject with more advanced chronic kidney disease (CKD) (eGFR>30 or 45). In an embodiment, the disorder is a post-transplant IgA nephropathy. In an embodiment, the disorder is a pediatric IgA nephropathy. In an embodiment, the disorder is Henoch-Schonlein purpura or cutaneous vasculitis). In an embodiment, the disorder is IgA nephropathy in a subject with crescentic glomerulonephritis (GN). In an embodiment, the disorder is IgA vasculitis. In an embodiment, the disorder is IgA bullous dermatosis. In an embodiment, the disorder is IgA dermatitis (e.g., IgA dermatitis herpetiformis, IgA bullous dermatosis). In an embodiment, the disorder is IgM mediated neuropathy (e.g., anti-MAG peripheral neuropathy or IgM mediated neuropathy associated with anti-GM1 antibodies). In an embodiment, the disorder is Waldenstrom's Macroglobulinemia (WM). In an embodiment, the disorder is lupus nephritis.

In some embodiments, a disease, disorder, or condition treatable or preventable with an antibody molecule as described herein is an autoimmune disorder (e.g., an autoantibody-related disorder, e.g., an IgM autoantibody-related disorder). In embodiments, the autoantibody-related disorder comprises primary membranous nephropathy, Goodpasture's disease, or cold agglutinin disease. In embodiments, a disease, disorder, or condition treatable or preventable with an antibody molecule as described herein is an IgM-mediated disorder (e.g., an IgM neuropathy). In embodiments, a disease, disorder, or condition treatable or preventable with an antibody molecule as described herein is a glomerulonephritis. In some embodiments, the subject is not immunocompromised. In an embodiment, the subject does not have decreased serum IgG levels relative to an average healthy subject (e.g., wherein the serum IgG levels of the subject are at least 50%, 60%, 70%, 75%, 80%, 85%, 90%, 95%, 96%, 97%, 98%, 99%, or 100% relative to an average healthy subject).

The antibody molecules described herein are typically administered at a frequency that keeps a therapeutically effective level of antibody molecules in the patient's system until the patient recovers. For example, the antibody molecules may be administered at a frequency that achieves a serum concentration sufficient for at least about 1, 2, 5, 10, 20, 30, or 40 antibody molecules to bind each APRIL molecule. In an embodiment, the antibody molecules are administered every 1, 2, 3, 4, 5, 6, or 7 days, every 1, 2, 3, 4, 5, or 6 weeks, or every 1, 2, 3, 4, 5, or 6 months.

Methods of administering various antibody molecules are known in the art and are described below. Suitable dosages of the antibody molecules used will depend on the age and weight of the subject and the particular drug used.

In an embodiment, the antibody molecule is administered to the subject (e.g., a human subject) intravenously. In an embodiment, the antibody molecule is administered to the subject at a dose between 0.1 mg/kg and 50 mg/kg, e.g., between 0.2 mg/kg and 25 mg/kg, between 0.5 mg/kg and 10 mg/kg, between 0.5 mg/kg and 5 mg/kg, between 0.5 mg/kg and 3 mg/kg, between 0.5 mg/kg and 2.5 mg/kg, between 0.5 mg/kg and 2 mg/kg, between 0.5 mg/kg and 1.5 mg/kg, between 0.5 mg/kg and 1 mg/kg, between 1 mg/kg and 1.5 mg/kg, between 1 mg/kg and 2 mg/kg, between 1 mg/kg and 2.5 mg/kg, between 1 mg/kg and 3 mg/kg, between 1 mg/kg and 2.5 mg/kg, or between 1 mg/kg and 5 mg/kg. In an embodiment, the antibody molecule is administered to the subject at a dose of about 1, 2, 3, 4, 5, 6, 7, 8, 9, 10, 11, 12, 13, 14, 15, 16, 17, 18, 19, or 20 mg/kg. In an embodiment, the antibody molecule is administered to the subject at a dose of about 2, 4, or 8 mg/kg. In an embodiment, the antibody molecule is administered to the subject at a fixed dose between 10 mg and 1000 mg, e.g., between 10 mg and 500 mg, between 10 mg and 250 mg, between 10 mg and 150 mg, between 10 mg and 100 mg, between 10 mg and 50 mg, between 250 mg and 500 mg, between 150 mg and 500 mg, between 100 mg and 500 mg, between 50 mg and 500 mg, between 25 mg and 250 mg, between 50 mg and 150 mg, between 50 mg and 100 mg, between 100 mg and 150 mg. between 100 mg and 200 mg, or between 150 mg and 250 mg.

In an embodiment, the antibody molecule is administered to the subject at a dose of about 100 mg, about 200 mg, about 400 mg, about 600 mg, about 800 mg, or about 1000 mg, or about 1200 mg, e.g., subcutaneously.

In an embodiment, the antibody molecule is administered subcutaneously to the subject (e.g., the human subject). In an embodiment, the antibody molecule is administered subcutaneously at a dose of about 200 mg. In an embodiment, the antibody molecule is administered subcutaneously to the subject at a dose of about 400 mg. In an embodiment, the antibody molecule is administered subcutaneously to the subject at a dose of about 600 mg. In an embodiment, the antibody molecule is administered subcutaneously to the subject at a dose of about 800 mg. In an embodiment, the antibody molecule is administered at a unit dose of 100 mg/mL to 300 mg/mL, e.g., 200 mg/mL. In an embodiment, the antibody molecule is administered by a subcutaneous injection of about 0.5 mL to 5 mL, e.g., 1 mL, 1.5 mL, 2 mL, 2.5 mL, 3 mL, 3.5 mL, 4 mL, 4.5 mL, 5 mL, 5.5 mL, or 6 mL. In an embodiment, the antibody molecule is administered at a dose of 200 mg by one 1 mL subcutaneous injection. In an embodiment, the antibody molecule is administered at a dose of 400 mg by one 2 mL subcutaneous injection. In an embodiment, the antibody molecule is administered at a dose of 600 mg by one 2 mL subcutaneous injection and one 1 mL subcutaneous injection.

In some embodiments, the antibody molecule is administered once. In other embodiments, the antibody molecule is administered more than once. In certain embodiments, the antibody molecule is administered at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 times. In an embodiment, the antibody molecule is administered once a week, twice a week, once every two weeks, once every three weeks, once every four weeks, once every eight weeks, once a month, once every two months, or once every three months. In an embodiment, the antibody molecule is administered once a month, e.g., for a period of at least 1, 2, 3, 4, 5,6,7, 8,9, 10, 11, 12, 13, 14, 15, 16, 17, 18, 19, 20, 21, 22, 23, 24, 25, 26, 27, 28, 29, 30, 31, 32, 33, 34, 35, or 36 months. In an embodiment the antibody molecule is administered as a single subcutaneous dose, e.g., in a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 weeks. In an embodiment, the antibody molecule is administered between 0.5 mg/kg and 3 mg/kg or between 50 mg and 150 mg, once a week, twice a week, once every two weeks, or once every four weeks.

The antibody molecules can be used by themselves or conjugated to a second agent, e.g., a bacterial agent, toxin, or protein, e.g., a second anti-APRIL antibody molecule. This method includes: administering the antibody molecule, alone or conjugated to a second agent, to a subject requiring such treatment. The antibody molecules can be used to deliver a variety of therapeutic agents, e.g., a toxin, or mixtures thereof.

### IgA Nephropathy

IgA nephropathy (also known as Berger's disease, Berger disease, Berger's syndrome, Berger syndrome, IgA nephritis, IgAN, or synpharyngitic glomerulonephritis) is the most prevalent, chronic glomerular disease worldwide. Conservative epidemiological estimates cite a global incidence of approximately 5-50 cases/million (children) and 10-40 cases/million (adults). This incidence of disease presents a regional bias with a higher prevalence in Asia and the Americas, with a particularly higher disease burden in Japan and regions of China. Biopsy confirmed cases of IgA nephropathy in Japan are projected at approximately 350,000. In the US, this projection is approximately 100,000-as such, it is the most frequently diagnosed 1° glomerular disease in adults. While a relatively indolent disease, IgA nephropathy leads to end stage renal disease (ESRD), i.e., renal failure in 20-50% of patients within a 20-30 year span. These numbers are likely grossly underreported given the need to confirm the disease by kidney biopsy, a protocol that is variably practiced in various clinical settings. The disease has a complex pathogenesis with genetic, epidemiological, and potentially environmental components to disease etiology, pathology, and progression. It likewise has a variable clinical presentation ranging from asymptomatic to end-stage renal failure (ESRD). There are currently no disease-specific treatments to address primary disease or progression.

The etiology of this disease, as its name implies, has been established. In brief, the disease is caused by the deposition of IgA, typically in the form of immune complexes in the mesangium of the kidney. A molecular characterization of these particular immunoglobulins has been carried out. These IgAs are of the A1 subclass (IgA1 vs. IgA2), predominantly polymeric (with J chain-mediated linkages), and apparently differentially o-glycosylated in the hinge region that is intervening between CH1 and CH2 domains. In particular, these o-glycans are heterogeneously lacking 01,3 galactose linkages and, as such, are commonly referred to as galactose-deficient IgA1 (or gdIgA1). As the pathogenesis of this disease can involve a polygenic, multi-hit mechanism for inducing renal pathology and aberrant physiology, IgA1 may be viewed as the so-called auto-antigen representing this first critical “hit” in a multi-hit model for IgA nephropathy. A set of autoantibodies for this disease has likewise been defined and it relates to immunoglobulins (predominantly IgG) that specifically recognize this differentially glycosylated epitope and promote the formation of immune complexes (representing so-called “hit 2”). It should also be noted that IgA itself is subject to aggregation due to misfolding, conformational changes, and potential changes in the N-glycosylation state of the CH2/CH3 glycans.

Without wishing to be bound by theory, it is believed that in an embodiment, aberrantly glycosylated IgA1 levels correlate with disease and clinical outcomes in IgA nephropathy. Aberrantly glycosylated IgA1 has been characterized directly from kidney biopsies and increased production of aberrantly glycosylated IgA1 was observed in B cells (tonsillar, PBMC) in IgA nephropathy patients. The level of galactose-deficient IgA1 in the sera of patients with IgA nephropathy is associated with disease progression (Zhao et al. Kidney Int. 2012; 82(7):790-6). Differential lectin staining demonstrated elevated levels of aberrantly glycosylated IgA1 in serum and glomeruli of IgA nephropathy patients relative to healthy controls (Allen et al. Kidney Int. 2001; 60(3):969-73).

Based on this evolving disease model, IgA nephropathy may be appropriately viewed as an autoimmune disease with strong and critical extra-renal involvement. The identification and validation of select immune-based targets proposed to play a critical role in disease pathogenesis, namely the production of IgA and subsequent production of autoreactive antibodies to this target, represent a logical therapeutic strategy for treatment. APRIL (TNFSF13) represents particular area of focus for this reason.

Additional rationale for targeting APRIL include emerging genetic data based on multiple, comprehensive genome wide association (GWAS) studies along with IgA related genetic disorders e.g., IgA hypogammaglobulinemia related common variable immunoglobulin deficiency (CVID) whose locus maps to defects in TNFRSF13B (TACI) with direct implications of the role of APRIL-TACI interactions in regulating IgA synthesis.

IgA nephropathy often does not cause symptoms in the early stages. The disease can go unnoticed for years and is sometimes first diagnosed when routine tests reveal protein and red blood cells in urine that cannot be seen without a microscope (microscopic hematuria). Signs and symptoms of IgA nephropathy when kidney function is impaired include, e.g., cola- or tea-colored urine (caused by red blood cells in the urine); repeated episodes of cola- or tea-colored urine, sometimes even visible blood in the urine, usually during or after an upper respiratory or other type of infection; pain in the side(s) of the back below the ribs (flank); foam in the toilet water from protein in the urine; swelling (edema) in the hands and feet; and high blood pressure. In an embodiment, the sign or symptom includes, e.g., one or more of hematuria, proteinuria, albuminuria, hypertension, or an early stage kidney disease (e.g., requiring dialysis or transplantation). In an embodiment, the sign or symptom is associated with, e.g., one or more of aberrantly glycosylated IgA1, auto-antibody formation, deposition of nephritogenic immune complexes in the kidney, or inflammation and loss of kidney function.

The classic presentation (in about 40-50% of the cases, more common in younger adults) of IgA nephropathy is episodic hematuria which usually starts within a day or two of a non-specific upper respiratory tract infection (hence synpharyngitic). Less commonly gastrointestinal or urinary infection can be the inciting agent. All of these infections have in common the activation of mucosal defenses and hence IgA antibody production. These episodes can occur on an irregular basis every few months and in most patients eventually subsides. Renal function usually remains normal, though rarely, acute kidney failure may occur.

A smaller proportion (in about 20-30% of the cases, usually the older population) of IgA nephropathy patients have microscopic hematuria and proteinuria (less than 2 gram/day). These patients may not have any symptoms and are only clinically found if a doctor decides to take a urine sample. Hence, the disease is more commonly diagnosed in situations where screening of urine is compulsory, e.g., school children in Japan.

Some (about 5% each) IgA nephropathy patients have the following disease presentation: nephrotic syndrome (e.g., 3-3.5 grams of protein loss in the urine, associated with a poorer prognosis); acute kidney failure (e.g., either as a complication of the frank hematuria, when it usually recovers, or due to rapidly progressive glomerulonephritis which often leads to chronic kidney failure); chronic kidney failure (e.g., no previous symptoms, presents with anemia, hypertension and other symptoms of kidney failure, in people who probably had longstanding undetected microscopic hematuria and/or proteinuria).

A variety of systemic diseases can be associated with IgA nephropathy such as liver failure, celiac disease, rheumatoid arthritis, reactive arthritis, ankylosing spondylitis and HIV. Diagnosis of IgA nephropathy and a search for any associated disease occasionally reveals such an underlying serious systemic disease. Occasionally, there are simultaneous symptoms of Henoch-Schonlein purpura. Some HLA alleles have been suspected along with complement phenotypes as being genetic factors.

IgA nephropathy can be diagnosed by various tests, e.g., urine test, blood tests (e.g., to show increased blood levels of the waste product creatinine), iothalamate clearance test, kidney imaging (e.g., ultrasound, X-rays, or cystoscopy), kidney biopsy, or a combination thereof.

For an adult patient with isolated hematuria, tests such as ultrasound of the kidney and cystoscopy are usually done first to pinpoint the source of the bleeding. These tests would rule out kidney stones and bladder cancer, two other common urological causes of hematuria. In children and younger adults, the history and association with respiratory infection can raise the suspicion of IgA nephropathy. A kidney biopsy is often necessary to confirm the diagnosis. The biopsy specimen shows proliferation of the mesangium, with IgA deposits on immunofluorescence and electron microscopy. However, patients with isolated microscopic hematuria (i.e., without associated proteinuria and with normal kidney function) are not usually biopsied since this is associated with an excellent prognosis. A urinalysis will show red blood cells, usually as red cell urinary casts. Proteinuria, usually less than 2 grams per day, also may be present. Other renal causes of isolated hematuria include, e.g., thin basement membrane disease and Alport syndrome, the latter being a hereditary disease associated with hearing impairment and eye problems. Other blood tests done to aid in the diagnosis include CRP or ESR, complement levels, ANA, and LDH. Protein electrophoresis and immunoglobulin levels can show increased IgA in 50% of all patients.

Treatment with a number of medications can slow the progress of the disease and help manage symptoms such as high blood pressure, protein in the urine (proteinuria), and swelling (edema) in the hands and feet. Exemplary therapies for IgA nephropathy include, e.g., high blood pressure medications (e.g., angiotensin-converting enzyme (ACE) inhibitors or angiotensin receptor blockers (ARBs)), omega-3 fatty acids, immunosuppressants (e.g., corticosteroid medications, such as prednisone), statin therapy, mycophenolate mofetil, ciclosporin, mizoribine, cyclophosphamide (e.g., in combination with anti-platelet/anticoagulants, or in combination with steroids and azathioprine), kidney dialysis, or kidney transplantation. Exemplary therapies for IgA nephropathy are also described in Floege and Eitner J Am. Soc. Nephrol. 22: 1785-1794, 2011. Other exemplary therapies for IgA nephropathy are described in the section of “Combination Therapies” herein.

Without wishing to be bound by theory, it is believed that in an embodiment, targeting APRIL selectively reduces IgA. APRIL−/− mice have normal T and B lymphocyte development, normal T and B cell proliferation in vitro, but decreased serum IgA levels (Castigli et al. Proc Natl Acad Sci USA. 2004; 101(11):3903-8). Discovery of new risk loci for IgA nephropathy implicates genes involved in immunity against intestinal pathogens (Kiryluk et al. Nat Genet. 2014; 46(11):1187-96). Serum levels and B cell production of APRIL are elevated in patients with IgA nephropathy and correlate with aberrantly glycosylated IgA levels (Zhai et al. Medicine (Baltimore). 2016; 95(11):e3099). Plasma levels of APRIL (TNFSF13) correlate with progression of chronic kidney disease in IgA nephropathy (Han et al. JAm Soc Nephrol. 2016; 27(2):439-53). Treatment with anti-APRIL antibody results in reduction of serum IgA, clearing of kidney mesangium, and reduction of inflammatory cell infiltration and glomerular injury, in mice (Kim et al. PLoS One. 2015; 10(9):e0137044). Anti-APRIL antibody preserves immune cell homeostasis in bone marrow and spleen (Kim et al. PLoS One. 2015; 10(9):e0137044).

APRIL (TNFSF13) represents a logical biological and therapeutic target for the treatment of IgA nephropathy. Without wishing to be bound by theory, it is believed that in an embodiment, the efficacy of the antibody molecules described herein with respect to the targeted modulation of APRIL-mediated immunobiolgocial mechanisms is directly relevant to treatment of IgA nephropathy. The anti-APRIL antibody molecules described herein (e.g., humanized anti-APRIL antibody molecules), e.g., with high biological potency and/or low complement activation, can be used to treat IgA nephropathy. In an embodiment, the antibody molecule has picomolar APRIL binding affinity and sub-nanomolar receptor blocking activity to both TACI and BCMA, e.g., in vitro. In another embodiment, the antibody molecule functionally interfere with APRIL mediated downstream cellular signaling, e.g., through the canonical NFκB activation pathway. In an embodiment, the antibody molecule is engineered, e.g., as an IgG2 subtype, for purposes of clinically mitigating against antibody-dependent exacerbation of complement recruitment, e.g., in the kidneys of IgA nephropathy patients. In an embodiment, an antibody molecule described herein can have an improved safety profile in comparison to more depletive B cell-based therapeutic approaches, e.g., due to a lesser perturbation of B and T cell homeostasis as shown in a murine model (Kim et al. PLoS One. 2015;10(9):e0137044).

The antibody molecules described herein can be used to treat or prevent different stages of IgA nephropathy. In an embodiment, the antibody molecule is used to treat a symptom associated with IgA nephropathy, e.g., hematuria, proteinuria, albuminuria, hypertension, an early stage kidney disease (e.g., requiring dialysis or transplantation), or a combination thereof. In an embodiment, the antibody molecule reduces aberrantly glycosylated IgA1, auto-antibody formation, deposition of nephritogenic immune complexes in the kidney, inflammation and loss of kidney function, or a combination thereof. In an embodiment, the subject is at low risk, e.g., having minor urinary abnormalities (e.g., micro-hematuria), normal glomerular filtration rate (GFR), and/or no hypertension. In another embodiment, the subject is at moderate to high risk, e.g., having proteinuria greater than 0.5-1 g/d and/or GFR reduced (e.g., below 30-50 ml/min) and/or hypertension. In yet another embodiment, the subject has acute or rapid GFR loss, e.g., having nephrotic syndrome or rapidly progressive glomerulonephritis (RPGN), or acute kidney injury (AKI) due to macro-hematuria or other common cause. In an embodiment, the subject has proteinuria greater than 0.5 g/day, e.g., between 0.5-1 g/day or greater than 1 g/day. In an embodiment, the subject treated for IgA nephropathy has glomerular filtration rate (GFR) less than 50 ml/min, e.g., less than 30 ml/min.

The antibody molecules described herein can be used to treat various forms of IgA nephropathy or a disorder or condition associated with IgA nephropathy. In an embodiment, the subject treated for IgA nephropathy has more advanced chronic kidney disease (CKD), wherein the estimated GFR (eGFR)>30 or 45. In an embodiment, the subject treated for IgA nephropathy has crescentic glomerulonephritis (GN). In an embodiment, the antibody molecule is used to treat a pediatric IgA nephropathy. In an embodiment, the antibody molecule is used to treat a post-transplant IgA nephropathy. In an embodiment, the antibody molecule is used to treat Henoch-Schonlein purpura (HSP) or cutaneous vasculitis. In an embodiment, the antibody molecule does not significantly change (e.g., capable of preserving) immune cell homeostasis. In another embodiment, the antibody molecule results in a reduction of IgA not total ablation of IgA.

### Diabetic Nephropathy

The antibody molecule described herein can be used to treat or prevent diabetic nephropathy. Diabetic nephropathy (or known as diabetic kidney disease) is a progressive kidney disease caused, e.g., by damage to the capillaries in the kidneys' glomeruli. It is typically characterized by nephrotic syndrome and diffuse scarring of the glomeruli. It is often due to longstanding diabetes mellitus, and is a prime reason for dialysis. It is classified as a small blood vessel complication of diabetes.

Exemplary symptoms of diabetic nephropathy include, but are not limited to, severe tiredness, headaches, a general feeling of illness, nausea, vomiting, frequent voiding, lack of appetite, itchy skin, or leg swelling. The cause of diabetic nephropathy can include, e.g., high blood sugar, advanced glycation end product formation. Cytokines may be involved in the development of diabetic nephropathy.

Diabetes can cause a number of changes to the body's metabolism and blood circulation, which likely combine to produce excess reactive oxygen species. These changes damage the kidney's glomeruli, which leads to the hallmark feature of albuminuria (Cao and Cooper J Diabetes Investig. 2011; 2(4): 243-247). As diabetic nephropathy progresses, the glomerular filtration barrier (GFB), which is composed of the fenestrated endothelium, glomerular basement membrane, and epithelial podocytes, is increasingly damaged (Mora-Femindez et al. J. Physiol. (Lond.) 2014; 592 (Pt 18): 3997-4012). Damage to the glomerular basement membrane allows proteins in the blood to leak through, leading to accumulation in Bowman's space as distinct periodic-acid schiff positive nodules (Kimmelstiel-Wilson nodules).

Diagnosis of diabetic nephropathy can be based on the measurement of high levels of albumin in the urine or evidence of reduced kidney function (Lewis and Maxwell Practitioner. 2014; 258(1768):13-7, 2). Albumin measurements can be defined as follows: normal albuminuria: urinary albumin excretion<30 mg/24h; microalbuminuria: urinary albumin excretion in the range of 30-299 mg/24h; clinical (overt) albuminuria: urinary albumin excretion>300 mg/24h. To test kidney function, the person's estimated glomerular filtration rate (eGFR) is measured from a blood sample. Normal eGFR ranges from 90 to 120 ml/min/1.73 m².

Other treatments that can be used in combination with the antibody molecule described herein to treat diabetic nephropathy include, e.g., an angiotensin-converting enzyme (ACE) inhibitor (e.g., captopril, enalapril, lisinopril, or ramipril), an angiotensin II receptor blocker (ARB) (e.g., candesartan cilexetil, irbesartan, losartan, or telmisartan), a calcium channel blocker (e.g., amlodipine, diltiazem, or verapamil), a diuretic (e.g., chlorthalidone, hydrochlorothiazide, or spironolactone), a beta-blocker (e.g., atenolol, carvedilol, or metoprolol), and diabetes management (e.g., control of high blood pressure or blood sugar levels, or reduction of dietary salt intake).

### Cancer

The antibody molecule described herein can be used to treat or prevent a cancer. Exemplary cancers that can be treated or prevented by the antibody molecules described herein include, but are not limited to, acute lymphoblastic leukemia (ALL), acute myeloid leukemia (AML), adrenocortical carcinoma, Kaposi sarcoma, an AIDS-related lymphoma, primary central nervous system (CNS) lymphoma, anal cancer, appendix cancer, astrocytoma, atypical teratoid/rhabdoid tumor, basal cell carcinoma, bile duct cancer, bladder cancer, bone cancer (e.g., Ewing sarcoma or osteosarcoma and malignant fibrous histiocytoma), brain tumor (e.g., astrocytomas, brain stem glioma, central nervous system atypical teratoid/rhabdoid tumor, central nervous system embryonal tumor, central nervous system germ cell tumor, craniopharyngioma, or ependymoma), breast cancer, bronchial tumor, Burkitt lymphoma, carcinoid tumor (e.g., gastrointestinal carcinoid tumor), cardiac (heart) tumor, embryonal tumor, germ cell tumor, lymphoma, cervical cancer, cholangiocarcinoma, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma in situ (DCIS), endometrial cancer, ependymoma, esophageal cancer, esthesioneuroblastoma, Ewing sarcoma, extracranial germ cell tumor, extragonadal germ cell tumor, eye cancer (e.g., intraocular melanoma or retinoblastoma), fallopian tube cancer, fibrous histiocytoma of bone, osteosarcoma, gallbladder cancer, gastric (stomach) cancer, gastrointestinal carcinoid tumor, gastrointestinal stromal tumors (GIST), germ cell tumor (e.g., central nervous system tumor, extracranial tumor, extragonadal tumor, ovarian cancer, or testicular cancer), gestational trophoblastic disease, glioma, hairy cell leukemia, head and neck cancer, hepatocellular (liver) cancer, Hodgkin lymphoma, hypopharyngeal cancer, intraocular melanoma, islet cell tumor, pancreatic neuroendocrine tumor, Kaposi sarcoma, kidney cancer (e.g., renal cell cancer or Wilms tumor), Langerhans cell histiocytosis (LCH), laryngeal cancer, leukemia (e.g., acute lymphoblastic leukemia (ALL), acute myeloid leukemia (AML), chronic lymphocytic leukemia (CLL), chronic myelogenous leukemia (CML), or hairy cell leukemia), lip and oral cavity cancer, liver cancer, lung cancer (e.g., non-small cell lung cancer (NSCLC) or small cell lung cancer), lymphoma (e.g., aids-related, Burkitt lymphoma, cutaneous T-cell lymphoma, Hodgkin lymphoma, non-Hodgkin lymphoma, or primary central nervous system (CNS) lymphoma), Waldenstrom macroglobulinemia, male breast cancer, malignant fibrous histiocytoma of bone and osteosarcoma, melanoma (e.g., intraocular (eye) melanoma), Merkel cell carcinoma, mesothelioma, metastatic squamous neck cancer, midline tract carcinoma, mouth cancer, multiple endocrine neoplasia syndrome, multiple myeloma/plasma cell neoplasm, mycosis fungoides, myelodysplastic syndrome, myelodysplastic/myeloproliferative neoplasm, chronic myeloproliferative neoplasm, nasal cavity and paranasal sinus cancer, nasopharyngeal cancer, neuroblastoma, oral cancer, lip and oral cavity cancer, oropharyngeal cancer, osteosarcoma and malignant fibrous histiocytoma of bone, ovarian cancer (e.g., epithelial ovarian cancer or germ cell ovarian tumor), pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, pleuropulmonary blastoma, peritoneal cancer, prostate cancer, rectal cancer, retinoblastoma, rhabdomyosarcoma, salivary gland cancer, sarcoma (e.g., Ewing sarcoma, Kaposi sarcoma, osteosarcoma, rhabdomyosarcoma, soft tissue sarcoma, or uterine sarcoma), Sézary syndrome, skin cancer (e.g., melanoma, Merkel cell carcinoma, or nonmelanoma skin cancer), small intestine cancer, squamous cell carcinoma, testicular cancer, throat cancer, thymoma and thymic carcinoma, thyroid cancer, transitional cell cancer of the renal pelvis and ureter, urethral cancer, endometrial uterine cancer, vaginal cancer, vulvar cancer, or a metastatic lesion thereof.

In an embodiment, the cancer is a hematological cancer, e.g., a lymphoma or leukemia, e.g., chosen from B-cell non-Hodgkin's lymphoma, chronic lymphocytic leukemia (CLL), Hodgkin's lymphoma, multiple myeloma, Waldenstrom macroglobulinemia, or lymphoplasmacytic lymphoma. In an embodiment, the cancer is a multiple myeloma. In another embodiment, the cancer is a solid tumor, e.g., chosen from colorectal cancer, breast cancer (e.g., breast carcinoma), esophageal cancer (e.g., esophageal adenocarcinoma), brain cancer (e.g., glioblastoma), or kidney cancer (e.g., renal cell carcinoma).

In an embodiment, the antibody molecule is used to treat a lymphoma. Other treatments that can be used in combination with the antibody molecule described herein to treat lymphoma include, e.g., chemotherapy, immunotherapy, targeted drug therapy, radiation therapy, and stem cell transplant. Exemplary targeted drug therapy includes a CD20 inhibitor (e.g., rituximab (RITUXAN® or ibritumomab tiuxetan (ZEVALIN®)).

In an embodiment, the antibody molecule is used to treat a leukemia. Other treatments that can be used in combination with the antibody molecule described herein to treat leukemia include, e.g., chemotherapy, immunotherapy, targeted drug therapy, radiation therapy, and stem cell transplant. Exemplary targeted drug therapy includes a tyrosine kinase inhibitor (e.g., imatinib (GLEEVEC®).

In an embodiment, the antibody molecule is used to treat a multiple myeloma. Other treatments that can be used in combination with the antibody molecule described herein to treat multiple myeloma include, e.g., chemotherapy, corticosteroids, immunotherapy, targeted drug therapy, radiation therapy, and stem cell transplant. Exemplary targeted drug therapy includes, e.g., a thalidomide analog (e.g., thalidomide (THALOMID®), lenalidomide (REVLIMID®), or pomalidomide (POMALYST®)).

In an embodiment, the antibody molecule is used to treat Waldenstrom macroglobulinemia.

Other treatments that can be used in combination with the antibody molecule described herein to treat Waldenstrom macroglobulinemia include, e.g., plasma exchange, chemotherapy, immunotherapy, targeted drug therapy, and stem cell transplant.

In an embodiment, the antibody molecule is used to treat a colorectal cancer. Other treatments that can be used in combination with the antibody molecule described herein to treat colorectal cancer include, e.g., surgery, chemotherapy, radiation therapy, immunotherapy, and targeted drug therapy.

Exemplary targeted drug therapy includes, e.g., a VEGF inhibitor (e.g., bevacizumab (AVASTIN®)), an EGFR inhibitor (e.g., cetuximab (ERBITUX®), panitumumab (VECTIBIX®)), and dual VEGFR2-TIE2 tyrosine kinase inhibitor (e.g., regorafenib (STIVARGA®)).

In an embodiment, the antibody molecule is used to treat a breast cancer, e.g., a breast carcinoma. Other treatments that can be used in combination with the antibody molecule described herein to treat breast cancer include, e.g., surgery, chemotherapy, radiation therapy, hormone therapy, immunotherapy, and targeted drug therapy. Exemplary target drug therapy includes, e.g., an HER2 inhibitor (e.g., trastuzumab (HERCEPTIN®), pertuzumab (PERJETA®), ado-trastuzumab (KADCYLA®), or lapatinib (TYKERB®)) or a VEGF inhibitor (e.g., bevacizumab (AVASTIN®)).

In an embodiment, the antibody molecule is used to treat an esophageal cancer, e.g., an esophageal adenocarcinoma. Other treatments that can be used in combination with the antibody molecule described herein to treat esophageal cancer include, e.g., surgery, chemotherapy, radiation therapy, and immunotherapy.

In an embodiment, the antibody molecule is used to treat a brain cancer, e.g., a glioblastoma. Other treatments that can be used in combination with the antibody molecule described herein to treat brain cancer include, e.g., surgery, chemotherapy, radiation therapy, radiosurgery, immunotherapy, and targeted drug therapy. Exemplary targeted drug therapy includes, e.g., a VEGF inhibitor (e.g., bevacizumab (AVASTIN®)).

In an embodiment, the antibody molecule is used to treat a kidney cancer, e.g., a renal cell carcinoma. Other treatments that can be used in combination with the antibody molecule described herein to treat kidney cancer include, e.g., surgery, cryoablation, radiofrequency ablation, radiation therapy, immunotherapy, and targeted drug therapy. Exemplary targeted drug therapy includes, e.g., a VEGF inhibitor (e.g., bevacizumab (AVASTIN®)), a tyrosine kinase inhibitor (e.g., axitinib (INLYTA®), pazopanib (VOTRIENT®), sorafenib (NEXAVAR®), or sunitinib (SUTENT®), or an mTOR inhibitor (e.g., temsirolimus (TORISEL®) or everolimus (AFINITOR®).

Immunoprolferative Disorders The antibody molecule described herein can be used to treat or prevent an immunoproliferative disorder. Immunoproliferative disorders (also known as immunoproliferative diseases or immunoproliferative neoplasms) are disorders of the immune system that are characterized by the abnormal proliferation of the primary cells of the immune system (e.g., B cells, T cells and Natural killer (NK) cells) or by the excessive production of immunoglobulins (e.g., antibodies).

Exemplary immunoproliferative disorders include, but are not limited to, lymphoproliferative disorders (LPDs), hypergammaglobulinemia, and paraproteinemia. Lymphoproliferative disorders include several conditions in which lymphocytes are produced in excessive quantities. They typically occur in patients who have compromised immune systems. Hypergammaglobulinemia is often characterized by increased levels of immunoglobulins in the blood serum. Paraproteinemia or monoclonal gammopathy is the presence of excessive amounts of a single monoclonal gammaglobulin (e.g., a paraprotein) in the blood. In an embodiment, the antibody molecule is used to treat monoclonal IgA hypergammaglobulinemia.

### Vasculitis

The antibody molecule described herein can be used to treat or prevent vasculitis. Vasculitis is a group of disorders that destroy blood vessels by inflammation. Vasculitis is primarily caused by leukocyte migration and resultant damage. Exemplary types of vasculitis include, but are not limited to, microscopic polyarteritis (poly-angiitis), Wegener's granulomatosis, Henoch Schonlein purpura and polyarteritis nodosa.

In an embodiment, the antibody molecule is used to treat IgA vasculitis. In an embodiment, the antibody molecule is used to treat Henoch-Schonlein purpura (IgA associated vasculitis).

Henoch-Schonlein purpura (HSP, also known as anaphylactoid purpura, purpura rheumatica, or Schonlein-Henoch purpura) is a disease of the skin and other organs that most commonly affects children. HSP is a systemic vasculitis (inflammation of blood vessels) and is characterized by deposition of immune complexes of IgA and complement component 3 (C3) on arterioles, capillaries, and venules. In the skin, the disease causes palpable purpura (small hemorrhages); often with joint and abdominal pain. With kidney involvement, there may be a loss of small amounts of blood and protein in the urine; in a small proportion of cases, the kidney involvement proceeds to chronic kidney disease even irreversible kidney damage. HSP is often preceded by an infection, such as a throat infection.

Symptoms of Henoch-Schonlein purpura include, e.g., rash (purpura), swollen or sore joints (arthritis), gastrointestinal symptoms (e.g., abdominal pain, nausea, vomiting or bloody stools), and kidney involvement (e.g., protein or blood in the urine). Serum levels of IgA are high in HSP patients.

Standards for defining Henoch-Schonlein purpura include, e.g., the 1990 American College of Rheumatology (ACR) classification (Mills et al. (1990). Arthritis and Rheumatism 33 (8): 1114-21), the 1994 Chapel Hill Consensus Conference (CHCC) (Jennette et al. (1994) Arthritis and Rheumatism 37 (2): 187-92), and the 2006 European League Against Rheumatism (EULAR) and Pediatric Rheumatology Society (PReS) classification, which includes palpable purpura as a mandatory criterion, together with at least one of the following findings: diffuse abdominal pain, predominant IgA deposition (confirmed on skin biopsy), acute arthritis in any joint, and renal involvement (as evidenced by the presence of blood and/or protein in the urine) (Ozen et al. (2006) Annals of Rheumatic Diseases 65 (7): 936-41).

Other treatments that can be used in combination with the antibody molecule described herein to treat Henoch-Schonlein purpura include, e.g., analgesics for the abdominal and joint pains, steroids (e.g., oral steroids or a combination of intravenous methylprednisolone (steroid), cyclophosphamide and dipyridamole followed by prednisone). Other regimens also include, e.g., steroids/azathioprine, and steroids/cyclophosphamide (with or without heparin and warfarin), or intravenous immunoglobulin (IVIG).

In another embodiment, the antibody molecule is used to treat acute proliferative glomerulonephritis, e.g., post-streptococcal glomerulonephritis.

Acute proliferative glomerulonephritis is a disorder of the glomeruli (glomerulonephritis), or small blood vessels in the kidneys. It is a common complication of bacterial infections, typically skin infection by Streptococcus bacteria types 12, 4 and 1 (impetigo) but also after streptococcal pharyngitis, for which it is also known as postinfectious or poststreptococcal glomerulonephritis. The infection causes blood vessels in the kidneys to develop inflammation, which hampers the renal organs ability to filter urine.

The pathophysiology of this disorder is consistent with an immune complex mediated mechanism. This disorder produces proteins that have different antigenic determinants, which in turn have an affinity for sites in the glomerulus. As soon as binding occurs to the glomerulus, via interaction with properdin, complement is activated. Complement fixation causes the generation of additional inflammatory mediators.

Symptoms of acute proliferative glomerulonephritis include, e.g., hematuria, oliguria, edema, hypertension, fever, headache, malaise, anorexia, and nausea.

Other treatments that can be used in combination with the antibody molecule described herein to treat cute proliferative glomerulonephritis includes, e.g., blood pressure (BP) control and control of the amount of potassium in individuals with oliguric acute kidney injury.

### Autoimmune Disorders

The antibody molecule described herein can be used to treat or prevent an autoimmune disorder. Exemplary autoimmune disorders that can be treated or prevented by the antibody molecule described herein include, but are not limited to, acute Disseminated Encephalomyelitis (ADEM), acute necrotizing hemorrhagic leukoencephalitis, Addison's disease, agammaglobulinemia, alopecia areata, amyloidosis, ankylosing spondylitis, anti-GBM/anti-TBM nephritis, antiphospholipid syndrome (APS), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenic purpura (ATP), autoimmune thyroid disease, autoimmune urticaria, axonal & neuronal neuropathies, Balo disease, Behcet's disease, bullous pemphigoid, cardiomyopathy, Castleman disease, celiac disease, Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogans syndrome, cold agglutinin disease, congenital heart block, coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, Erythema nodosum, experimental allergic encephalomyelitis, Evans syndrome, fibromyalgia, fibrosing alveolitis, giant cell arteritis (temporal arteritis), giant cell myocarditis, glomerulonephritis, Goodpasture's syndrome, granulomatosis with polyangiitis (GPA) (formerly called Wegener's Granulomatosis), Graves' disease, Guillain-Barre syndrome, Hashimoto's encephalitis, Hashimoto's thyroiditis, hemolytic anemia, Henoch-Schonlein purpura, Herpes gestationis, hypogammaglobulinemia, idiopathic thrombocytopenic purpura (ITP), IgA nephropathy, IgG4-related sclerosing disease, immunoregulatory lipoproteins, inclusion body myositis, interstitial cystitis, juvenile arthritis, juvenile diabetes (type 1 diabetes), juvenile myositis, Kawasaki syndrome, Lambert-Eaton syndrome, Leukocytoclastic vasculitis, Lichen planus, Lichen sclerosus, Ligneous conjunctivitis, linear IgA disease (LAD), pupus (SLE), Lyme disease, chronic, Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, Pars planitis (peripheral uveitis), pemphigus, peripheral neuropathy, perivenous encephalomyelitis, pernicious anemia, POEMS syndrome, polyarteritis nodosa, Type I, II, & III autoimmune polyglandular syndromes, polymyalgia rheumatica, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, raynauds phenomenon, reactive arthritis, reflex sympathetic dystrophy, reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, subacute bacterial endocarditis (SBE), Susac's syndrome, sympathetic ophthalmia, Takayasu's arteritis, temporal arteritis/Giant cell arteritis, thrombocytopenic purpura (TTP), Tolosa-Hunt syndrome, transverse myelitis, Type 1 diabetes, ulcerative colitis, undifferentiated connective tissue disease (UCTD), uveitis, vasculitis, vesiculobullous dermatosis, vitiligo, Wegener's granulomatosis (also known as Granulomatosis with Polyangiitis (GPA).

In an embodiment, the autoimmune disorder is rheumatoid arthritis, systemic lupus erythematosus, a linear IgA bullous disease (e.g., linear immunoglobulin A (IgA) dermatosis), or IgA-mediated epidermolysis bullosa acquisita.

In an embodiment, the antibody molecule is used to treat rheumatoid arthritis. Other treatments that can be used in combination with the antibody molecule described herein to treat rheumatoid arthritis includes, e.g., an NSAID, a steroid (e.g., corticosteroid), a disease-modifying antirheumatic drug (DMARD) (e.g., methotrexate (TREXALL®), leflunomide (ARAVA®), hydroxychloroquine (PLAQUENIL®), or sulfasalazine (AZULFIDINE®), a biologic response modifier (e.g., abatacept (ORENCIA®), adalimumab (HUMIRA®), anakinra (KINERET®), certolizumab (CIMZIA®), etanercept (ENBREL®), golimumab (SIMPONI®), infliximab (REMICADE®), rituximab (RITUXAN®) and tocilizumab (ACTEMRA®), or Tofacitinib (XELJANZ®)), or surgery.

In an embodiment, the antibody molecule is used to treat systemic lupus erythematosus (SLE). Other treatments that can be used in combination with the antibody molecule described herein to treat SLE, e.g., an NSAID, an antimalarial drug (e.g., hydroxychloroquine (PLAQUENIL®), corticosteroid (e.g., prednisone), an immunosuppressant (e.g., azathioprine (IMURAN®, AZASAN®), mycophenolate (CELLCEPT®), leflunomide (ARAVA®), or methotrexate (TREXALL®)), or a BAFF inhibitor (e.g., belimumab (BENLYSTA®).

In an embodiment, the antibody molecule is used to treat a linear IgA bullous disease (e.g., linear immunoglobulin A (IgA) dermatosis). Other treatments that can be used in combination with the antibody molecule described herein to treat a linear IgA bullous disease (e.g., linear immunoglobulin A (IgA) dermatosis) include, e.g., corticosteroids (e.g., prednisone orprednisolone), an antibiotic (e.g., tetracycline, erythromycin, sulfapyridine), colchicine, or mycophenolate mofetil.

In an embodiment, the antibody molecule is used to treat IgA-mediated epidermolysis bullosa acquisita. Other treatments that can be used in combination with the antibody molecule described herein to treat IgA-mediated epidermolysis bullosa acquisita includes, e.g., an antibiotic, an anti-inflammatory drug (e.g., corticosteroid), or surgery.

### Linear IgA Bullous Dermatosis

The antibody molecule described herein can be used to treat or prevent IgA dermatitis, e.g., linear IgA bullous disease/linear immunoglobulin A (IgA) dermatosis, and IgA-mediated epidermolysis bullosa acquisita. Linear IgA bullous dermatosis is a mucocutaneous autoimmune disease characterized by linear deposition of IgA and disruption of the dermoepidermal junction. In an embodiment, linear IgA bullous dermatosis is an autoimmune reaction against basement membrane proteins such as the lamina lucida and sublamina densa. The basement membrane anchors the epidermis to the dermis and helps to stabilize the skin. When IgA antibodies target such proteins, the basement membrane destabilizes resulting in tense blister formation. In an embodiment, linear IgA bullous dermatosis is drug-induced (e.g., induced from antibiotics (e.g., vancomycin), antihypertensives, and nonsteroidal anti-inflammatory agents), associated with another disease or disorder (e.g., a lymphoproliferative disorder, infection, ulcerative colitis, or systemic lupus (SLE)). In an embodiment, linear IgA bullous dermatosis can be of idiopathic origin. In children, the lesions of linear IgA bullous dermatosis are generally localized to the lower abdomen, perineal area, and inner thighs. In adults, the lesions are generally localized to the extensor surfaces, trunk, buttocks, and face.

Exemplary symptoms of linear IgA bullous dermatosis include, but are not limited to, prodromal itching, burning and discharge from the eyes, formation of tense blisters on the base of red or normal-appearing skin, clustering of blisters that results in the classic cluster of jewels sign or linearly along the edge of a blister resulting in the string of beads sign, and/or scattered red bumps or plaques at sites of inflammation.

Linear IgA bullous dermatosis can be diagnosed using clinical, immunological, and histopathological tests. A skin biopsy of a blister is typically performed, and immunofluorescence can also be done, to detect the presence of IgA deposits along the basement membrane zone in a linear pattern, which is typically indicative of linear IgA bullous dermatosis.

Other treatments that can be used in combination with the antibody molecule described herein to treat linear IgA bullous dermatosis include, e.g., dapsone, sulfonamides, sulfapyridine, mycophenolate mofetil, corticosteroids (e.g., prednisone or prednisolone), colchicine), an antibiotic (e.g., tetracycline, erythromycin, sulfapyridine), nicotinamide, or surgery.

### IgM Mediated Neuropathy

The antibody molecule described herein can be used to treat or prevent IgM mediated neuropathy, e.g., anti-myelin associated glycoprotein (MAG) peripheral neuropathy or IgM mediated neuropathy associated with anti-GM1 antibodies. In an embodiment, the antibody molecule described herein can be used to treat or prevent anti-MAG. Anti-MAG neuropathy is characterized by the development of autoantibodies to myelin associated glycoprotein (MAG), which is found in the myelin sheath and in Schwann cells. These autoantibodies can impair MAG function and neuronal signaling, leading to loss of nerve function and problems with sensory and motor function. In an embodiment, anti-MAG neuropathy results from monoclonal gammopathy, e.g., IgM monoclonal gammopathy.

Exemplary symptoms of anti-MAG neuropathy include, but are not limited to, sensory loss, e.g., sensory loss in toes and fingers, loss of vibration sensation, unsteady gait, tremors in hands and legs, or weakness.

Anti-MAG neuropathy can be diagnosed using clinical features, electrodiagnostic studies, and measurement of serum IgM protein levels.

Other treatments that can be used in combination with the antibody molecule described herein to treat anti-MAG neuropathy, include cyclophosphamide, rituximab (RITUXAN®), plasma exchange, or intravenous immunoglobulin (IvIg).

In an embodiment, the antibody molecule described herein can be used to treat or prevent IgM mediated neuropathy associated with anti-GM1 ganglioside antibodies, e.g., multifocal motor neuropathy (MMN). MMN is characterized by progressive asymptomatic muscle weakness and atrophy. In an embodiment, a subject treated for MMN has IgM anti GMI1 ganglioside antibodies. Exemplary symptoms include, but are not limited to, functional motor deficit, ganglioside accumulation, increased CSF protein, muscle spasm, reduced tendon reflexes, progressive muscle weakness, weakness in the hands and lower arms, cramping, involuntary contractions or twitching, wrist drop or foot drop, or atrophy of affected muscles. In an embodiment, MMN results from an abnormal immune response.

MMN can be diagnosed using clinical features, electrodiagnostic studies, and measurement of serum IgM protein levels. Other treatments that can be used in combination with the antibody molecule described herein to treat MMN include intravenous immunoglobulin (IvIg), rituximab (RITUXAN®), cyclophosphamide, or physical therapy.

### Waldenstrom's Macroglobulinemia

The antibody molecule described herein can be used to treat or prevent Waldenstrom's macroglobulinemia. Waldenstrom's macroglobulinemia is a hematological cancer typically characterized by an excess of lymphoplasmacytic cells in the bone marrow. In an embodiment, Waldenstrom's macroglobulinemia is classified as a lymphoplasmacytic lymphoma. These abnormal cells generally comprise characteristics of lymphocytes and B cells and demonstrate aberrant expression of IgM, e.g., they produce excess IgM. In an embodiment, the excess IgM can build up in various organs, e.g., the heart and/or the kidneys, resulting in amyloidosis. In an embodiment, accumulation of lymphoplasmacytic cells in different tissues can result in hepatomegaly, splenomegaly, or enlarged lymph nodes.

Waldenstrom's macroglobulinemia can be a slow growing lymphoma. In an embodiment, Waldenstrom's macroglobulinemia can be clinically insignificant and indolent. In an embodiment, Waldenstrom's macroglobulinemia can be clinically significant. In an embodiment, Waldenstrom's macroglobulinemia results from a combination of genetic mutations, e.g., a mutation in the MYD88 gene and/or the CXCR4 gene.

Exemplary symptoms of Waldenstrom's macroglobulinemia include, but are not limited to, easily bruising, nose bleeds, bleeding of the gums, fatigue, weight loss, peripheral neuropathy (numbness in hands and feet), anemia, fever, headache, shortness of breath, changes in vision (e.g., blurring or loss of vision), dizziness, ataxia, cryoglobulinemia, confusion, or night sweats.

Waldenstrom's macroglobulinemia can be diagnosed by a blood test to detect, e.g., blood count, IgM protein levels in the blood, and/or measure organ function, e.g., kidney and/or liver function. Waldenstrom's macroglobulinemia can also be diagnosed and/or prognosed using a bone marrow biopsy and/or imaging tests, e.g., a CT scan or a PET scan.

Other treatments that can be used in combination with the antibody molecule described herein to treat Waldenstrom macroglobulinemia include, e.g., plasma exchange, chemotherapy, immunotherapy, targeted drug therapy, and stem cell transplant.

### Lupus Nephritis

The antibody molecule described herein can be used to treat or prevent lupus nephritis. Lupus nephritis is an autoimmune disorder that is a form of glomerulonephritis that can constitute the most severe organ manifestation of systemic lupus erythematosus (SLE). Lupus nephritis leads to autoantibodies in the kidney which causes inflammation, e.g., inflammation in the nephrons, and impairs kidney function, e.g., waste removal and filtration. It can result in permanent scarring and damage to the kidneys and possibly end-stage renal disease (ESRD). Lupus nephritis often develops in a subject within five years of developing lupus.

Exemplary symptoms of lupus nephritis include, but are not limited to, blood in the urine (hematuria), proteinuria, foamy urine (e.g., foamy urine due to excess protein in the urine), increased urination, edema, joint pain, high blood pressure, swelling in hands, ankles, and feet, excess levels of creatinine in the blood, muscle pain, weight gain, fever of unknown etiology, red rash that is typically localized to the face (e.g., across the nose and face).

Diagnosis of lupus nephritis can be based on urinalysis and the measurement of blood, cell casts (e.g., cell fragments often found in the blood and/or the tubules of the kidneys), and protein levels in the urine. Diagnosis can also be based on a blood test to estimate kidney function, e.g., a creatinine blood test with or without a blood urea nitrogen (BUN) test. Additionally, to test kidney function, the person's estimated glomerular filtration rate (eGFR) can be measured from a blood sample. A kidney biopsy can also be performed, which can be used to stage lupus nephritis. In an embodiment, lupus nephritis is classified as one of six stages under the International Society of Nephrology/Renal Pathology Society (ISN/RPS) classification system, which include, minimal mesangial glomerulonephritis (Class I), mesangial proliferative lupus nephritis (Class II), focal lupus nephritis (<50% glomeruli) (Class III), diffuse segmental or global lupus nephritis (>50% glomeruli) (Class IV), membranous nephritis (Class V), or advanced sclerosing lupus nephritis (Class VI).

Other treatments that can be used in combination with the antibody molecule described herein to treat lupus nephritis, including but not limited to cyclophosphamide, mycophenolate mofetil, calcineurin inhibitors (e.g., azathioprine or tacrolimus), cyclosporine A, hydroxychloroquine, rituximab (RITUXAN®), belimumab, dialysis, kidney transplant, corticosteroids angiotensin-converting enzyme (ACE) inhibitors with angiotensin receptor blockers (ARBs), diuretics, beta blockers, and/or calcium channel blockers.

### Other Disorders

The antibody molecule described herein can be used to treat or prevent other disorders, e.g., IgA pemphigus, celiac disease, or alcoholic cirrhosis.

In an embodiment, the antibody molecule is used to treat or prevent IgA pemphigus. Other treatments that can be used in combination with the antibody molecule described herein to treat IgA pemphigus include, e.g., corticosteroid, an immunosuppressant (e.g., azathioprine (IMURAN®), methotrexate (TREXALL®), or mycophenolate mofetil (CELLCEPT®)), an CD-20 inhibitor (e.g., rituximab (RITUXAN®), an antibiotic, an antiviral agent, or an antifungal agent.

In an embodiment, the antibody molecule is used to treat or prevent celiac disease. Other treatments that can be used in combination with the antibody molecule described herein to treat celiac disease include, e.g., a gluten-free diet, a vitamin or mineral supplement, or a steroid.

In an embodiment, the antibody molecule is used to treat or prevent alcoholic cirrhosis. Other treatments that can be used in combination with the antibody molecule described herein to treat alcoholic cirrhosis include, e.g., an immunosuppressant (e.g., azathioprine, prednisone, azathioprine, cyclosporine, or methotrexate) or liver transplant.

### Combination Therapies

The antibody molecules can be used in combination with other therapies. For example, the combination therapy can include an antibody molecule co-formulated with, and/or co-administered with, one or more additional therapeutic agents, e.g., one or more additional therapeutic agents described herein. In other embodiments, the antibody molecules are administered in combination with other therapeutic treatment modalities, e.g., other therapeutic treatment modalities described herein. Such combination therapies may advantageously utilize lower dosages of the administered therapeutic agents, thus avoiding possible toxicities or complications associated with the various monotherapies.

Administered “in combination”, as used herein, means that two (or more) different treatments are delivered to the subject before, or during the course of the subject's affliction with a disorder. In an embodiment, two or more treatments are delivered prophylactically, e.g., before the subject has the disorder or is diagnosed with the disorder. In another embodiment, the two or more treatments are delivered after the subject has developed or diagnosed with the disorder. In some embodiments, the delivery of one treatment is still occurring when the delivery of the second begins, so that there is overlap. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, the additional agent is a second antibody molecule, e.g., an antibody molecule different from a first antibody molecule. In some embodiments, the second antibody molecule is an anti-APRIL antibody molecule (e.g., a different anti-APRIL antibody molecule from the first antibody molecule). Exemplary antibody molecules that can be used in combination include, but are not limited to, any combination of the antibody molecules listed in Table 1 or 5. In an embodiment, the second antibody molecule is sibeprenlimab, or a functional fragment, variant, or derivative thereof.

In an embodiment, the antibody molecule is administered in combination with a TACI-IgG fusion protein. In an embodiment, the antibody molecule is administered in combination with telitacicept, or a functional fragment, variant, or derivative thereof.

In an embodiment, the antibody molecule is administered in combination with a C1 inhibitor (e.g., a C1s inhibitor). In an embodiment, the antibody molecule is administered in combination with sutimlimab, or a functional fragment, variant, or derivative thereof.

In an embodiment, the antibody molecule is administered in combination with an anti-CD20 antibody molecule. In an embodiment, the antibody molecule is administered in combination with rituximab, or a functional fragment, variant, or derivative thereof.

In an embodiment, the antibody molecule is administered in combination with a second therapy to treat or prevent IgA nephropathy. In an embodiment, the antibody molecule is administered in combination with a second therapy to treat or prevent IgA nephropathy with crescentic glomerulonephritis (GN).

In an embodiment, the antibody molecule is administered in combination with an angiotensin-converting-enzyme (ACE) inhibitor or an angiotensin receptor blocker (ARB).

In an embodiment, the antibody molecule is administered in combination with an Fc decoy receptor, e.g., a soluble Fc receptor. In an embodiment, the soluble Fc receptor is a soluble Fc-gamma receptor IIB. In an embodiment, the soluble Fc receptor is SM101/BAX 1810 (Baxalta). In an embodiment, the soluble Fc receptor is administered at a dose between 1 mg/kg and 50 mg/kg, e.g., between 5 mg/kg and 15 mg/kg, between 12 mg/kg and 24 mg/kg, or between 20 mg/kg and 30 mg/kg.

In an embodiment, the antibody molecule is administered in combination with repository corticotropin (ACTHAR®). Repository corticotropin is an adrenocorticotropic hormone (ACTH) analogue. In an embodiment, repository corticotropin is administered at a dose between 50 U and 150 U, e.g., between 80 U and 120 U, by subcutaneous injection, twice or three times a week. In an embodiment, repository corticotropin is administered at a dose of 120 U, by subcutaneous injection, e.g., once, twice, or three times a week.

In an embodiment, the antibody molecule is administered in combination with mycophenolate mofetil (MMF). Mycophenolate mofetil is the 2-morpholinoethyl ester of mycophenolic acid (MPA), an immunosuppressive agent and inosine monophosphate dehydrogenase (IMPDH) inhibitor. In an embodiment, mycophenolate mofetil is administered at a dose of between 0.5 g and 2 g, e.g., between 1 g and 1.5 g or between 1.5 g and 2 g, orally or intravenously, e.g., once, twice, or three times a day.

In an embodiment, the antibody molecule is administered in combination with bortezomib (VELCADE®). Bortezomib, also known as [(1R)-3-methyl-1-({(2S)-3-phenyl-2-[(pyrazin-2-ylcarbonyl)amino]propanoyl}amino)butyl]boronic acid, is a proteasome inhibitor. In an embodiment, bortezomib is administered at a dose at between 0.5 mg/m² and 2.5 mg/m², e.g., between 1 mg/m² and 1.5 mg/m², e.g. every three days or every week.

In an embodiment, the antibody molecule is administered in combination with allopurinol (ZYLOPRIM®). Allopurinol, also known as 1H-pyrazolo[3,4-d]pyrimidin-4(2H)-one, is a purine analog.

In an embodiment, allopurinol is administered at a dose between about 50 mg and 1000 mg, e.g., between 100 mg and 600 mg or between 200 and 300 mg, orally, e.g., once a day or once two days.

In an embodiment, the antibody molecule is administered in combination with prednisone and/or cyclophosphamide. In an embodiment, prednisone is administered at a dose between 0.2 mg/kg and 2 mg/kg, e.g., between 0.5 mg/kg and 1 mg/kg, e.g., once a day. In an embodiment, cyclophosphamide is administered at a dose between 0.2 g and 2 g, e.g., between 0.5 g and 1 g, e.g., once a day.

In an embodiment, the antibody molecule is administered in combination with rituximab (RITUXAN®). Rituximab is a chimeric anti-CD20 monoclonal antibody. In an embodiment, rituximab is administered at a dose between 100 mg/m² and 500 mg/m², e.g., between 200 mg/m² and 450 mg/m² or between 300 mg/m² and 400 mg/m², intravenously, e.g., once weekly, once every two weeks, once every four weeks, or once every eight weeks.

In an embodiment, the antibody molecule is administered in combination with blisibimod. Blisibimod, also known as A-623 or AMG 623, is a selective antagonist of B-cell activating factor (BAFF, also known as B-lymphocyte stimulator or BLyS).

In an embodiment, the antibody molecule is administered with budesonide. In an embodiment, the budesonide is NEFECON®, an oral formulation that releases budesonide.

In an embodiment, the antibody molecule is administered with valsartan and/or probucol. In an embodiment, valsartan is administered at a dose between 50 mg/day and 200 mg/day, e.g., between 80 mg/day and 160 mg/day. In an embodiment, probucol is administered at a dose between 500 mg/day and 1000 mg/day, e.g., between 700 mg/day and 800 mg/day.

In an embodiment, the antibody molecule is administered in combination with OPL-CCL2-LPM. OPL-CCL2-LPM is a recombinant fusion protein comprised of the human CCL2 (monocyte chemoattractant protein-1) chemokine fused to a truncated form of the enzymatically active A1 domain of Shigella dysenteriae holotoxin (SA1). In an embodiment, OPL-CCL2-LPM is administered at a dose between 0.001 mg/kg and 1 mg/kg, e.g., between 0.01 mg/kg and 0.5 mg/kg or 0.05 mg/kg and 0.1 mg/kg, e.g., intravenously.

In an embodiment, the antibody molecule is administered in combination with methylprednisolone. In an embodiment, methylprednisolone is administered at a dose between 0.1 mg/kg and 2 mg/kg/day, e.g., between 0.2 mg/kg/day and 1.5 mg/kg/day or 0.5 mg/kg/day and 1 mg/kg/day, e.g., orally.

In an embodiment, the antibody molecule is administered in combination with sirolimus. Sirolimus, also known as rapamycin, can inhibit IL-2 and other cytokines receptor-dependent signal transduction mechanisms, via action on mTOR, and thereby block activation of T and B cells. In an embodiment, sirolimus is administered at dose between 0.2 mg/day and 2 mg/day, e.g., between 0.5 mg/day and 1 mg/day.

In an embodiment, the antibody molecule is administered in combination with a renin-angiotensin system (RAS) blocker. For example, the RAS blocker can be an angiotensin-converting enzyme (ACE) inhibitor or an AT1 receptor blocker (ARB). Exemplary ACE inhibitors that can be used in combination with the antibody molecule described herein include, e.g., benazepril (LOTENSIN®), captopril, enalapril (VASOTEC®), fosinopril, lisinopril (ZESTRIL®), moexipril (UNIVASC®), perindopril (ACEON®), quinapril (ACCUPRIL®), ramipril (ALTACE®), or trandolapril (MAVIK®). Exemplary AT1 receptor blockers that can be used in combination with the antibody molecule described herein include, e.g., candesartan (ATACAND®), eprosartan (TEVETEN®), irbesartan (AVAPRO®), losartan (COZAAR®), olmesartan (BENICAR®), telmisartan (MICARDIS®), or valsartan (DIOVAN®).

In an embodiment, the antibody molecule is administered in combination with fostamatinib. Fostamatinib is a prodrug of the active compound tamatinib (R-406), which is an inhibitor of the enzyme spleen tyrosine kinase (Syk). In an embodiment, fostamatinib is administered at a dose between about 50 mg and 200 mg, e.g., between 100 mg and 150 mg, e.g., orally, e.g., every day.

In an embodiment, the antibody molecule is administered in combination with paricalcitol. In an embodiment, paricalcitol is administered at a dose between about 0.2 mg and 2 mg, e.g., between 0.5 mg and 1 mg, e.g., every day.

In an embodiment, the antibody molecule is administered in combination with ramipril. In an embodiment, ramipril is administered at a dose between about 0.5 mg and 5 mg, e.g., between 1 mg and 4 mg or between 2 mg and 3 mg, e.g., every day.

In an embodiment, the antibody molecule is administered in combination with an angiotensin-converting-enzyme (ACE) inhibitor. In an embodiment, the ACE inhibitor is enalapril (VASOTEC®).

In an embodiment, the antibody molecule is administered in combination with an immunosuppressant. In an embodiment, the immunosuppressant is tacrolimus. Tacrolimus, also known as FK-506 or fujimycin, is a macrolide calcineurin inhibitor.

In an embodiment, the antibody molecule is administered in combination with omega-3 fatty acids.

In an embodiment, the antibody molecule is administered in combination with CCX168. CCX168 is an orally administered C5aR inhibitor.

Exemplary therapies that can be used in combination with an antibody molecule or composition described herein to treat or prevent other disorders are also described in the section of “Methods of Treating or Preventing Disorders” herein.

### Methods of Diagnosis

In some aspects, the present disclosure provides a diagnostic method for detecting the presence of APRIL in vitro (e.g., in a biological sample, such as a biopsy or blood sample) or in vivo (e.g., in vivo imaging in a subject). The method includes: (i) contacting the sample with an antibody molecule described herein, or administering to the subject, the antibody molecule; (optionally) (ii) contacting a reference sample, e.g., a control sample (e.g., a control biological sample, such as a biopsy or blood sample) or a control subject with an antibody molecule described herein; and (iii) detecting formation of a complex between the antibody molecule and APRIL in the sample or subject, or the control sample or subject, wherein a change, e.g., a statistically significant change, in the formation of the complex in the sample or subject relative to the control sample or subject is indicative of the presence of APRIL in the sample. The antibody molecule can be directly or indirectly labeled with a detectable substance to facilitate detection of the bound or unbound antibody. Suitable detectable substances include various enzymes, prosthetic groups, fluorescent materials, luminescent materials and radioactive materials, as described above and described in more detail below.

The term “sample,” as it refers to samples used for detecting a polypeptide (e.g., APRIL) or a nucleic acid encoding the polypeptide includes, but is not limited to, cells, cell lysates, proteins or membrane extracts of cells, body fluids such as blood, or tissue samples such as biopsies.

Complex formation between the antibody molecule, and APRIL, can be detected by measuring or visualizing either the antibody molecule bound to APRIL or unbound antibody molecule. Any suitable detection assays can be used, and conventional detection assays include an enzyme-linked immunosorbent assays (ELISA), a radioimmunoassay (RIA) or tissue immunohistochemistry. Alternative to labeling the antibody molecule, the presence of APRIL can be assayed in a sample by a competition immunoassay utilizing standards labeled with a detectable substance and an unlabeled antibody molecule. In this assay, the biological sample, the labeled standards and the antibody molecule are combined and the amount of labeled standard bound to the unlabeled binding molecule is determined. The amount of APRIL in the sample is inversely proportional to the amount of labeled standard bound to the antibody molecule.

The antibody molecules described herein can be used to diagnose disorders that can be treated or prevented by the antibody molecules described herein. The detection or diagnostic methods described herein can be used in combination with other methods described herein to treat or prevent a disorder described herein.

### Other Aspects and Embodiments

In an aspect, the disclosure features a composition, e.g., pharmaceutical composition, comprising an antibody molecule described herein. In an embodiment, the composition further comprises a pharmaceutical acceptable carrier.

In an aspect, the disclosure features a nucleic acid molecule encoding a heavy chain variable region (VH), a light chain variable region (VL), or both, of an antibody molecule described herein. In an embodiment, the nucleic acid molecule encodes a heavy chain (HC), a light chain (LC), or both, of an antibody molecule described herein. In an aspect, the disclosure features a vector comprising a nucleic acid molecule described herein. In an aspect, the disclosure features a cell, e.g., an isolated cell, comprising a nucleic acid molecule described herein or a vector described herein.

In an aspect, the disclosure features a kit comprising an antibody molecule described herein and instructions to use of the antibody molecule.

In an aspect, the disclosure features a container comprising an antibody molecule described herein.

In an aspect, the disclosure features a method of producing an anti-APRIL antibody molecule, the method comprising culturing a cell described herein under conditions that allow production of an antibody molecule, thereby producing the antibody molecule.

In an embodiment, the method further comprises isolating the antibody molecule.

In an aspect, the disclosure features a method of treating IgA nephropathy, the method comprising administering to a subject in need thereof an effective amount of an antibody molecule described herein or a composition described herein, thereby treating IgA nephropathy.

In an embodiment, the antibody molecule is administered to the subject intravenously.

In an embodiment, the antibody molecule is administered to the subject at a dose between 0.1 mg/kg and 50 mg/kg, e.g., between 0.2 mg/kg and 25 mg/kg, between 0.5 mg/kg and 10 mg/kg, between 0.5 mg/kg and 5 mg/kg, between 0.5 mg/kg and 3 mg/kg, between 0.5 mg/kg and 2.5 mg/kg, between 0.5 mg/kg and 2 mg/kg, between 0.5 mg/kg and 1.5 mg/kg, between 0.5 mg/kg and 1 mg/kg, between 1 mg/kg and 1.5 mg/kg, between 1 mg/kg and 2 mg/kg, between 1 mg/kg and 2.5 mg/kg, between 1 mg/kg and 3 mg/kg, between 1 mg/kg and 2.5 mg/kg, or between 1 mg/kg and 5 mg/kg.

In an embodiment, the antibody molecule is administered to the subject at a fixed dose between 10 mg and 1000 mg, e.g., between 10 mg and 500 mg, between 10 mg and 250 mg, between 10 mg and 150 mg, between 10 mg and 100 mg, between 10 mg and 50 mg, between 250 mg and 500 mg, between 150 mg and 500 mg, between 100 mg and 500 mg, between 50 mg and 500 mg, between 25 mg and 250 mg, between 50 mg and 150 mg, between 50 mg and 100 mg, between 100 mg and 150 mg. between 100 mg and 200 mg, or between 150 mg and 250 mg.

In an embodiment, the antibody molecule is administered once a week, twice a week, once every two weeks, once every three weeks, once every four weeks, once every eight weeks, once a month, once every two months, or once every three months.

In an embodiment, administration of the antibody molecule reduces the level of IgA in a peripheral tissue, e.g., in serum, mucosal tissue, bone marrow, or any combination thereof.

In an embodiment, administration of the antibody molecule reduces the level of IgA1. In an embodiment, administration of the antibody molecule reduces the level of IgA1 in polymeric form (pIgA1). In an embodiment, administration of the antibody molecule reduces the level of IgA1 with 0-linked glycosylation variants (e.g., aberrant or reduced composition of galactose in CH1 hinge region).

In an embodiment, the method further comprises determining the level of IgA in a peripheral tissue sample from the subject, e.g., chosen from serum, mucosal tissue, or bone marrow.

In an embodiment, the method further comprises administering to the subject a second therapy for IgA nephropathy. In an embodiment, the second therapy is chosen from an angiotensin-converting enzyme (ACE) inhibitor, an angiotensin receptor blocker (ARB), omega-3 fatty acids, an immunosuppressant (e.g., a corticosteroid, e.g., prednisone), a statin, mycophenolate mofetil, or any combination thereof.

In an aspect, the disclosure features a method of treating diabetic nephropathy, the method comprising administering to a subject in need thereof an effective amount of an antibody molecule described herein or a composition described herein, thereby treating diabetic nephropathy.

In an aspect, the disclosure features a method of treating cancer, the method comprising administering to a subject in need thereof an effective amount of an antibody molecule described herein or a composition described herein, thereby treating cancer.

In an embodiment, the cancer is a hematological cancer. In an embodiment, the hematological cancer is chosen from B-cell non-Hodgkin's lymphoma, chronic lymphocytic leukemia (CLL), Hodgkin's lymphoma, multiple myeloma, Waldenstrom macroglobulinemia, or lymphoplasmacytic lymphoma. In an embodiment, the cancer is a multiple myeloma.

In an aspect, the disclosure features a method of treating an immunoproliferative disorder, the method comprising administering to a subject in need thereof an effective amount of an antibody molecule described herein or a composition described herein, thereby treating the immunoproliferative disorder.

In an embodiment, the immunoproliferative disorder is monoclonal IgA hypergammaglobulinemia.

In an aspect, the disclosure features a method of treating vasculitis, the method comprising administering to a subject in need thereof an effective amount of an antibody molecule described herein or a composition described herein, thereby treating vasculitis.

In an embodiment, the vasculitis is kidney vasculitis. In an embodiment, the vasculitis is an IgA associated vasculitis (e.g., Henoch-Schonlein purpura) or post-streptococcal glomerulonephritis.

In an aspect, the disclosure features a method of treating an autoimmune disorder, the method comprising administering to a subject in need thereof an effective amount of an antibody molecule described herein or a composition described herein, thereby treating the autoimmune disorder.

In an embodiment, the autoimmune disorder is chosen from rheumatoid arthritis, systemic lupus erythematosus, a linear IgA bullous disease (e.g., linear immunoglobulin A (IgA) dermatosis), or IgA-mediated epidermolysis bullosa acquisita (EBA).

In an aspect, the disclosure features a method of treating IgA pemphigus, the method comprising administering to a subject in need thereof an effective amount of an antibody molecule described herein or a composition described herein, thereby treating IgA pemphigus.

In an aspect, the disclosure features a method of treating celiac disease, the method comprising administering to a subject in need thereof an effective amount of an antibody molecule described herein or a composition described herein, thereby treating celiac disease.

In an aspect, the disclosure features a method of treating alcoholic cirrhosis, the method comprising administering to a subject in need thereof an effective amount of an antibody molecule described herein or a composition described herein, thereby treating alcoholic cirrhosis.

In an aspect, the disclosure features a method of reducing the level of IgA in a cell or subject, the method comprising contacting the cell or subject, or administering to a subject in need thereof an effective amount of, an antibody molecule described herein or a composition described herein, thereby reducing the level of IgA.

In an embodiment, the antibody molecule is administered to the subject intravenously.

In an embodiment, the antibody molecule is administered to the subject at a dose between 0.1 mg/kg and 50 mg/kg, e.g., between 0.2 mg/kg and 25 mg/kg, between 0.5 mg/kg and 10 mg/kg, between 0.5 mg/kg and 5 mg/kg, between 0.5 mg/kg and 3 mg/kg, between 0.5 mg/kg and 2.5 mg/kg, between 0.5 mg/kg and 2 mg/kg, between 0.5 mg/kg and 1.5 mg/kg, between 0.5 mg/kg and 1 mg/kg, between 1 mg/kg and 1.5 mg/kg, between 1 mg/kg and 2 mg/kg, between 1 mg/kg and 2.5 mg/kg, between 1 mg/kg and 3 mg/kg, between 1 mg/kg and 2.5 mg/kg, or between 1 mg/kg and 5 mg/kg.

In an embodiment, the antibody molecule is administered to the subject at a fixed dose between 10 mg and 1000 mg, e.g., between 10 mg and 500 mg, between 10 mg and 250 mg, between 10 mg and 150 mg, between 10 mg and 100 mg, between 10 mg and 50 mg, between 250 mg and 500 mg, between 150 mg and 500 mg, between 100 mg and 500 mg, between 50 mg and 500 mg, between 25 mg and 250 mg, between 50 mg and 150 mg, between 50 mg and 100 mg, between 100 mg and 150 mg. between 100 mg and 200 mg, or between 150 mg and 250 mg.

In an embodiment, the antibody molecule is administered once a week, twice a week, once every two weeks, once every three weeks, once every four weeks, once every eight weeks, once a month, once every two months, once every three months.

In an embodiment, administration of the antibody molecule reduces the level of IgA in a peripheral tissue, e.g., in serum, mucosal tissue, bone marrow, or any combination thereof.

In an embodiment, administration of the antibody molecule reduces the level of IgA1. In an embodiment, administration of the antibody molecule reduces the level of IgA1 in polymeric form (pIgA1). In an embodiment, administration of the antibody molecule reduces the level of IgA1 with 0-linked glycosylation variants (e.g., aberrant or reduced composition of galactose in CH1 hinge region).

In an aspect, the disclosure features use of an antibody molecule described herein or a composition described herein in the treatment, or in the manufacture of a medicament for the treatment, of a disorder described herein.

In another aspect, the disclosure features an antibody molecule described herein or a composition described herein for use in the treatment of a disorder described herein.

In an aspect, the disclosure features a method of detecting an APRIL molecule, the method comprising contacting a cell or a sample from a subject with an antibody molecule described herein, thereby detecting the APRIL molecule.

In an embodiment, the antibody molecule is coupled with a detectable label. In an embodiment, the APRIL molecule is detected in vitro or ex vivo. In another embodiment, the APRIL molecule is detected in vivo.

### EXAMPLES

### Example 1: Effect of Anti-APRIL Antibody Molecule on Serum Aberrantly Glycosylated IgA Levels

In this study, upon administration of an exemplary anti-APRIL antibody molecule, mAb 2419-1406, a dose-dependent reduction from baseline in serum aberrantly glycosylated Ig A (a-g IgA) levels was observed, and a dose response was generally observed in time to nadirs.

### Results

Following antibody dosing, a dose-dependent reduction from baseline in serum a-g IgA by timepoint levels was observed. The maximum mean (median) percent reduction in serum a-g IgA ranged from −24.35% (−28.13%) in the 0.5 mg/kg group (corresponding to 3.554 [2.730] g/mL observed) to −71.61% (−68.94%) in the 12.0 mg/kg group (corresponding to 1.920 [1.750] g/mL observed), and a dose response was generally observed in time to nadirs, which occurred at Week 4 for the 0.5 mg/kg group and Week 12 for the 12.0 mg/kg group (Table 10).

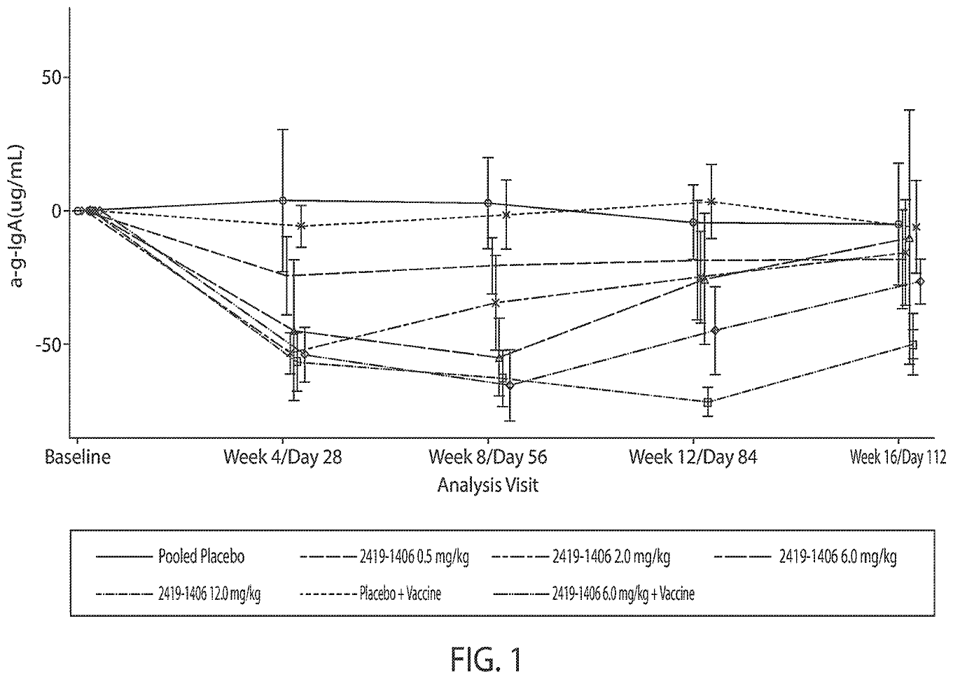

Serum a-g IgA level suppression was reversible, with a dose response in time to recovery during the follow-up period: the 0.5, 2.0, 6.0 and 12.0 mg/kg groups' mean (median) percent change at 16 weeks postdose were −18.16% (−21.43%), −15.41% (−8.25%), −9.93% (−21.21%) and −50.05% (−45.65%), respectively (Table 10). Overall, trends were generally similar for the Japanese and non-Japanese subjects as for the subjects overall (FIG. 1).

TABLE 10
Aberrantly Glycosylated Immunoglobulin A: Summary by Treatment-Any Ethnicity
(Pharmacodynamic Population)

| | | Week 4 | | Week 8 | | Week 12 | | Week 16 | |
|---|---|---|---|---|---|---|---|---|---|
| Statistic | Baseline | Observed | % Change | Observed | % Change | Observed | % Change | Observed | % Change |

a-g IgA (μg/mL): placebo

| | | | | | | | | | |
|---|---|---|---|---|---|---|---|---|---|
| n | 8 | 8 | 8 | 8 | 8 | 8 | 8 | 8 | 8 |
| Mean | 5.658 | 5.909 | 3.80 | 5.806 | 2.95 | 5.289 | −4.29 | 5.355 | −4.94 |
| (Med) | (6.130) | (6.285) | (−1.65) | (5.545) | (0.61) | (5.570) | (−4.76) | (5.570) | (−10.37) |
| SD | 1.751 | 2.177 | 26.839 | 1.894 | 17.083 | 1.375 | 14.028 | 1.982 | 22.678 |
| Min, | 2.750, | 1.960, | −28.7, | 2.460, | −18.4, | 2.840, | −28.3, | 2.370, | −33.2, |
| Max | 8.280 | 8.970 | 51.8 | 7.910 | 32.5 | 6.870 | 16.2 | 8.400 | 42.1 |

a-g IgA (μg/mL): antibody 0.5 mg/kg

| | | | | | | | | | |
|---|---|---|---|---|---|---|---|---|---|
| n | 7 | 7 | 7 | 6 | 6 | 7 | 7 | 7 | 7 |
| Mean | 4.804 | 3.554 | −24.35 | 3.965 | −20.59 | 3.719 | −18.51 | 3.814 | −18.16 |
| (Med) | (4.100) | (2.730) | (−28.13) | (3.265) | (−20.13) | (3.050) | (−27.08) | (2.730) | (−21.43) |
| SD | 2.674 | 1.908 | 14.671 | 2.155 | 10.658 | 1.659 | 22.408 | 1.922 | 18.574 |
| Min, | 2.660, | 2.270, | −44.6, | 2.270, | −36.6, | 2.430, | −38.0, | 2.600, | −38.2, |
| Max | 10.700 | 7.690 | −5.3 | 8.120 | −5.9 | 6.680 | 20.0 | 7.700 | 10.8 |

a-g IgA (μg/mL): antibody 2.0 mg/kg

| | | | | | | | | | |
|---|---|---|---|---|---|---|---|---|---|
| n | 7 | 7 | 7 | 6 | 6 | 5 | 5 | 5 | 5 |
| Mean | 5.363 | 2.517 | −53.46 | 3.615 | −34.44 | 4.310 | −24.66 | 4.836 | −15.41 |
| (Med) | (5.160) | (2.580) | (−50.19) | (3.490) | (−32.16) | (4.170) | (−19.65) | (4.670) | (−8.25) |
| SD | 0.985 | 0.715 | 7.710 | 1.246 | 17.806 | 1.568 | 17.060 | 1.775 | 19.981 |
| Min, | 4.660, | 1.510, | −67.6, | 2.320, | −54.6, | 2.510, | −51.5, | 2.820, | −45.6, |
| Max | 7.540 | 3.870 | −44.5 | 5.310 | −8.9 | 6.740 | −10.6 | 7.560 | 1.7 |

a-g IgA (μg/mL): antibody 6 mg/kg

| | | | | | | | | | |
|---|---|---|---|---|---|---|---|---|---|
| n | 7 | 7 | 7 | 7 | 7 | 6 | 6 | 7 | 7 |
| Mean | 6.270 | 3.435 | −44.80 | 2.756 | −54.93 | 5.293 | −25.73 | 5.771 | −9.93 |
| (Med) | (6.170) | (4.060) | (−49.89) | (2.910) | (−56.52) | (5.495) | (−20.94) | (7.030) | (−21.21) |
| SD | 3.807 | 2.030 | 26.244 | 1.577 | 14.598 | 3.346 | 24.709 | 3.817 | 47.555 |
| Min, | 1.150, | 0.500, | −79.4, | 0.500, | −73.6, | 0.500, | −56.5, | 0.515, | −55.2, |
| Max | 11.800 | 5.280 | 6.0 | 4.620 | −26.1 | 9.830 | 11.0 | 10.700 | 83.6 |

a-g IgA (μg/mL): antibody 12 mg/kg

| | | | | | | | | | |
|---|---|---|---|---|---|---|---|---|---|
| n | 7 | 7 | 7 | 6 | 6 | 7 | 7 | 7 | 7 |
| Mean | 6.960 | 2.913 | −56.70 | 2.518 | −62.95 | 1.920 | −71.61 | 3.374 | −50.05 |
| (Med) | (7.980) | (2.590) | (−53.47) | (2.190) | (−66.72) | (1.750) | (−68.94) | (3.880) | (−45.65) |
| SD | 3.312 | 1.562 | 10.965 | 1.683 | 10.546 | 0.981 | 5.432 | 1.811 | 11.621 |
| Min, | 1.700, | 0.791, | −77.4, | 0.508, | −74.3, | 0.528, | −79.5, | 0.976, | −75.6, |
| Max | 11.600 | 5.610 | −42.1 | 5.600 | −48.1 | 3.670 | −64.1 | 6.350 | −42.5 |

a-g IgA (μg/mL): placebo^{[2]} + vaccine

| | | | | | | | | | |
|---|---|---|---|---|---|---|---|---|---|
| n | 5 | 5 | 5 | 5 | 5 | 5 | 5 | 5 | 5 |
| Mean | 4.910 | 4.510 | −5.62 | 4.684 | −1.38 | 5.008 | 3.23 | 4.510 | −5.79 |
| (Med) | (4.350) | (4.150) | (−4.75) | (4.890) | (−6.10) | (5.300) | (2.71) | (4.600) | (−14.37) |
| SD | 2.232 | 1.684 | 7.965 | 1.670 | 12.980 | 2.052 | 14.190 | 1.775 | 17.310 |
| Min, | 2.950, | 2.810, | −16.2, | 2.770, | −15, | 2.560, | −13.2, | 2.280, | −22.7, |
| Max | 8.280 | 6.940 | 5.9 | 7.040 | −12.4 | 7.680 | 21.8 | 7.090 | 18.6 |

a-g IgA (μg/mL): vaccine + antibody 6.0 mg/kg

| | | | | | | | | | |
|---|---|---|---|---|---|---|---|---|---|
| n | 10 | 9 | 9 | 9 | 9 | 9 | 9 | 9 | 9 |
| Mean | 5.706 | 2.920 | −53.91 | 2.241 | −65.54 | 3.502 | −44.93 | 4.511 | −26.24 |
| (Med) | (4.375) | (2.460) | (−57.87) | (1.510) | (−67.69) | (3.240) | (−41.36) | (3.100) | (−28.41) |
| SD | 3.314 | 1.809 | 10.334 | 1.604 | 13.381 | 2.214 | 16.720 | 2.484 | 8.524 |
| Min, | 1.960, | 1.030, | −63.9, | 0.594, | −80.7, | 0.961, | −76.7, | 2.110, | −34.2, |
| Max | 12.500 | 6.420 | −33.2 | 4.810 | −33.1 | 7.330 | −25.2 | 9.090 | −8.8 |

Max = maximum;
Med = median;
Min = minimum;
N = number of subjects in the PD Population;
n = number of subjects evaluated;
PD = pharmacodynamics;
SD = standard deviation
^{[1]}All subjects who received Placebo within Cohorts 1-4.
^{[2]}All subjects who received Placebo within Cohort 5.
^{[3]}% Change: Percent change from baseline.

### Analysis

In data from a single-ascending dose study of the exemplary antibody molecule in healthy participants, the antibody molecule reduced serum levels of IgA and aberrantly glycosylated IgA1 (a-g IgAB). Aberrantly glycosylated IgA₁was measured during the study with a validated ELISA using KM55 [rat anti-(a-g IgA1)] coated plates with bound standards and samples subsequently detected with anti-human IgA.

At baseline, a-g IgA levels ranged from 5.4 to 7.0.g/mL across cohorts (Table 11). In contrast, baseline total IgA levels ranged from 2076 to 2866 μg/mL. Thus, serum a-g IgA in this healthy population comprised roughly 0.19 to 0.27% o of total serum IgA at Baseline.

Following antibody dosing in the study, a dose-dependent reduction from baseline in serum a-g IgA(by timepoint levels was observed. At peak a-g IgA nadir, the ratio of serum a-g IgA to total IgA ranged from 0.17% to 0.23% of(total IgA at the corresponding timepoint.

These data demonstrate that a-g IgA comprised approximately 0.19% to 0.27% o of total IgA at baseline, at a-g IgA nadir, the ratio of a-g IgA to total IgA ranged from 0.17% to 0.23% o, and in healthy volunteers, 2149-1406 significantly lowers both total IgA and a-g IgA, but does not appreciably alter the ratio of aberrantly glycosylated IgA to total IgA.

TABLE 11
Changes in Serum IgA, a-g IgA, and a-g IgA to total IgA ratios Over Time

| | | | | | Lowest | Timepoint | | |
|---|---|---|---|---|---|---|---|---|
| | | | BL a-g | BL Ratio, | Observed | of Lowest | IgA | Ratio, |
| | BL IgA | BL IgA | IgA | a-g | a-g IgA | Observed | level | a-g |
| | (mg/dL) | (ug/mL) | (ug/mL) | IgA/IgA | (ug/mL) | a-g IgA | (ug/mL) | IgA/IgA |
| Pooled | 231.1 | 2311 | 5.7 | 0.0025 | 5.36 | Week 16 | 2300 | 0.0023 |
| PBO | | | | | | | | |
| (n = 8) | | | | | | | | |
| 0.5 mg/kg | 207.6 | 2076 | 4.8 | 0.0023 | 3.55 | Week 4 | 1843 | 0.0019 |
| antibody | | | | | | | | |
| (n = 7) | | | | | | | | |
| 2.0 mg/kg | 284.4 | 2844 | 5.4 | 0.0019 | 2.52 | Week 4 | 1507 | 0.0017 |
| antibody | | | | | | | | |
| (n = 7) | | | | | | | | |
| 6.0 mg/kg | 286.6 | 2866 | 6.3 | 0.0022 | 2.76 | Week 8 | 1237 | 0.0022 |
| antibody | | | | | | | | |
| (n = 7) | | | | | | | | |
| 12.0 | 261 | 2610 | 7 | 0.0027 | 1.92 | Week 12 | 1154 | 0.0017 |
| mg/kg | | | | | | | | |
| antibody | | | | | | | | |
| (n = 7) | | | | | | | | |
| Placebo + | 220.3 | 2203 | 5.7 | 0.0026 | 4.51 | Week 4 | 2208 | 0.0020 |
| vaccine | | | | | | | | |
| (n = 5) | | | | | | | | |
| antibody + | 226.6 | 2266 | 4.9 | 0.0022 | 2.24 | Week 8 | 1060 | 0.0021 |
| vaccine | | | | | | | | |
| (n = 10) | | | | | | | | |

### Example 2: Study to Assess Pharmacokinetics, Pharmacodynamics, Safety, and Tolerability of Subcutaneously Administered Anti-APRIL Antibody

This phase 1, open-label single ascending single dose (SAD) study assesses the pharmacokinetics (PK), safety and tolerability, and pharmacodynamics (PD) upon subcutaneous (SC) administration of an exemplary anti-APRIL antibody molecule, mAb 2419-1406, in healthy non-Japanese and Japanese male and female participants aged 18 to 55 years. The results can inform the design and dose selection of other studies and provide data relating to the treatment of IgAN.

### Inclusion Criteria:

Healthy participants for this study are chosen from 18-55-year-old males and females based on a pre-study medical evaluation (medical history, physical examination, vital signs, 12 lead electrocardiogram (ECG), and clinical laboratory evaluations). Participants must meet the following criteria at the time of initial screening: white blood cells (3,000 to 12,000/mm³), platelets (>150,000/mm³), Haemoglobin (>13 gm/dL for males and >11 gm/dL for females), estimated glomerular filtration rate (>80 mL/min/1.73 m²), serum creatinine (<1.25×upper limit of normal (ULN)), glucose (<115 mg/dL after 8 hours of fasting), serum IgG (>6.0 g/L for participants 18 years of age or >7.0 g/L for participants>19 years of age), serum IgM (>0.4 g/L), serum IgA (>0.4 g/L for participants 18 years of age or >0.8 g/L for participants>19 years of age) and body mass index (BMI) between 17-32 kg/m².

### Dosage:

The exemplary antibody molecule is administered subcutaneously (SC) at a starting dose of 200 mg in Cohort 1. Doses in Cohorts 2 and 3 may be administered as either one or two SC injections. The doses in Cohort 2 may be adjusted based on data from Cohort 1 and dosage for Cohort 3 may be adjusted based on data from both Cohort 1 and Cohort 2, with the total dose not to exceed 800 mg. The highest potential single dose is 800 mg administered SC (in the optional Cohort 3), which is approximately equivalent to 9.1 mg/kg based on a body weight of 70 kg and 80% bioavailability.

The starting dose was selected upon consideration of the antibody target and mechanism of action, in vitro/in vivo toxicology data, the no observed-adverse-effect level (NOAEL) observed in a toxicology study in cynomolgus monkeys, and data from a previous first in human study with the exemplary antibody molecule.

The exemplary dose levels for each cohort are summarized in Table 12.

TABLE 12
Exemplary Dose Level for Each Cohort

| Cohorts | Treatment |
|---|---|
| Cohort 1 | 200 mg mAb (1 mL injection) |
| Cohort 2 | 400 mg mAb (2 mL injection) |
| Cohort 3 | Not to exceed 800 mg mAb |

Doses in Cohorts 2 and 3 can be administered as either 1 or 2 SC injections with the total dose not to exceed 800 mg mAb. The potential dose of 400 mg mAb 2419-1406 administered SC for Cohort 2 may be further adjusted based on the data from Cohort 1 and a decision for Cohort 3 can be made based on data from Cohorts 1 and 2. Doses can be selected such that they do not exceed a maximum allowed dose of 800 mg. Doses in Cohort 2 and 3 can be administered as either 1 or 2 SC injections.

### Study Design:

The study is conducted in 3 sequential dosing cohorts. On Day 1, a single dose of the exemplary antibody molecule is administered SC in the morning approximately 30 minutes after a light meal. For the participants in Cohort 1, 2 participants are initially dosed and monitored for 24 hours potential unexpected adverse events (AEs). After a successful observation period, the remaining participants of Cohort I are subsequently be dosed. The dosing level of Cohort 2 participants can be modified based on the data from Cohort 1 study. Cohort 3 represents an optimal cohort which may assess an additional dose level. The dosing level of Cohort 3 can be decided based on data obtained from Cohort 1 and 2. The study intervention that is administered for each cohort in this study are outlined in Table 13.

TABLE 13
Identity of Study Interventions

| | Cohort 1 | | Cohort 2 | | Cohort 3 |
|---|---|---|---|---|---|
| Dose Formulation | Solution | | Solution | | Solution |
| Unit Dose Strength | 200 | mg/mL | 200 | mg/mL | 200 mg/mL |
| Dosage level | 200 | mg | 400 | mg | TBD (not to exceed 800 mg) |
| Volume of Injection | 1.0 | mL | 2.0 | mL | TBD |

| | | | |
|---|---|---|---|
| Route of Administration | SC | SC | SC |
| Injection Site | Abdomen | Abdomen | Abdomen |

Pharmacokinetic sampling starts on Day 1 and samples are collected until the final follow up visit on Day 112. Pharmacodynamic samples (total IgA, IgG, and IgM) and samples for APRIL, a-g IgA, anti-drug antibody (ADA), secretory immunoglobulin A (sIgA), and salivary IgA are also collected during the study.

Participants are monitored to detect AEs and SAEs during the study and followed appropriately to ensure resolution of AEs. The following conditions can result in in discontinuation of the study intervention and participant discontinuation/withdrawal: any AE of severe intensity and related causality or any SAE of related causality; symptomatic hypotension (systolic blood pressure<85 mmHg and/or 20 mmHg decrease in systolic blood pressure based on 3 systolic blood pressure measurement), tachycardia (heart rate≥120 beats per minute lasting longer than 30 minutes or with impaired consciousness), ALT≥3×ULN, AST≥3×ULN, serum creatinine>1.5×ULN, a decrease from baseline in hemoglobin concentration>3 g/dL, white blood cell count<1,500/mm³, and/or platelets<50,000/mm³.

### Sample Collection:

Whole blood samples of approximately 5 mL per timepoint (starting from day 1 to the final follow up visit at day 112) are collected for measurement of serum concentrations of the exemplary antibody molecule for pharmacokinetic studies. Pharmacodynamic samples (total immunoglobulin IgA, IgG, and IgM), and samples for APRIL, aberrantly glycosylated immunoglobulin A (a-g IgA), anti-drug antibody (ADA), secretory immunoglobulin A (sIgA), and salivary IgA are also collected during the study. Salivary IgG is also be collected per timepoint for measuring salivary IgG level. In particular, whole blood samples of approximately 3.5 mL for Ig levels and 5 mL for APRIL, a-g IgA, and sIgA are collected at various time points. For assessing the effect of the exemplary antibody molecule on PD parameters, serum is evaluated for Ig levels (total IgA, IgG and IgM). Serum is also be evaluated for APRIL, a-g IgA, and sIgA. Salivary IgA is also evaluated in saliva samples taken at various time points.

### Endpoint Parameters Measured:

For pharmacokinetics (PK), the following parameters are measured: Cmax (primary), AUC_{0-inf}(primary), AUC₀₋ₗₐₛₜ (primary), Cₗₐₛₜ (secondary), tₘₐₓ(secondary), Tₗₐₛₜ(secondary), t_{1/2}(secondary), AUC_{0-28d} (secondary), AUC_{0-L112d}(secondary), % AUCₑₓ(secondary), V/_{d/F}(secondary), and CL/F(secondary).

For safety, the following parameters are measured: physical examination, vital sign (blood pressure, pulse, respiratory rate, and oral temperature), clinical laboratory tests (hematology, clinical chemistry, urianlysis, and Anti-drug antibody response), assessment of injection site, pain visual analog scale and adverse events (AEs).

For pharmacodynamics (PD), the following parameters are measured: Change from baseline in total serum IgG, IgA, and IgM concentrations and time to recovery; changes in serum a-g IgA levels; changes in plasma or serum APRIL levels; changes in the Serum sIgA levels; changes in salivary IgA levels; relationships between PK and PD/exploratory endpoints.

The following additional parameters are also be measured: characterization of ADA levels; assess ADA status impact on mAb 2419-1406 PK parameters.

% AUCex=Percentage of AUC0-inf obtained by extrapolation; AUC=area under the concentration-time curve; AUC_{0-28d}=area under the concentration-curve from time zero to Day 28; AUC_{0-112d}=AUC from time zero to Day 112; AUC_{0 inf}=AUC from time zero extrapolated to infinite time; AUC₀₋ₗₐₛₜ=AUC from time zero to the last quantifiable concentration; CL/F=apparent clearance; Clₐₛₜ=time of last serum concentration; t_{1/2}=apparent terminal elimination half-life; sIgA=secretory immunoglobulin A; tlₐₛₜ=time of last quantifiable observed concentration; tₘₐₓ=time of maximum serum concentration; VAS=visual analog scale; and Vd/F=apparent volume of distribution.

### Analysis:

Serum are evaluated for Ig levels (total IgA, IgG and IgM) for assessing the effect of the exemplary antibody molecule on PD parameters. Serum are also evaluated for APRIL, a-g IgA, and sIgA. Data collected from the study are statistically analysed. Descriptive statistics for continuous variables are summarized by treatment group and include the number of participants, arithmetic mean, standard deviation, median, minimum and maximum; descriptive statistics for categorical data are summarized by treatment group using frequency counts and percentages. Descriptive statistics for PK parameters include number of observations, arithmetic mean, standard deviation, arithmetic percentage coefficient of variation (% CV), and geometric mean, median, geometric % CV, minimum and maximum. Data grouped by cohorts and overall (all cohorts) are analysed separately if deemed appropriate. All safety analyses are performed using the safety population.

Pharmacokinetic analysis is performed using data from all participants assigned to the PK population. Antibody serum concentrations is listed for each participant. Two sets of summary statistics of antibody serum concentrations are presented by dose level; the first combines the ethnicities and the second set stratifies each dose level by ethnicity group. Individual and mean antibody concentration-time profiles are plotted for each dose level in both linear and logarithmic scales. The PK parameters as described under parameters measured section are summarized by dose level for non-Japanese, Japanese, and all participants using descriptive statistics. The noncompartmental PK parameters are calculated using Phoenix WinNonlin® Version 8.0 or higher. Pharmacokinetic/PD modelling can be used for further characterization of data and reported separately.

Pharmacodynamics for total IgA, IgG, and IgM, and other relevant PD applicable markers in serum or appropriate matrix (using the raw data, change from baseline data and percent change from baseline, as appropriate) are summarized using descriptive statistics and plotted overtime. Baseline data are taken as the last measurement prior to dosing or if not available, the screening value is used instead. An exploratory PK/PD analysis between the serum concentrations of the study intervention and PD assessments can be performed, if deemed appropriate.

Serum samples are screened for antibodies binding to the exemplary antibody molecule (ADA) and the titre of confirmed positive samples is reported. A summary table of confirmed positive or negative ADA is presented, by treatment (dose of the antibody molecule), and by study day based on the safety population. In addition, ADA titres are summarized by treatment and study day. The impact of ADA response on PK parameters is also analysed.

### Clinical Laboratory Tests

The tests detailed in Table 14 are performed.

TABLE 14
Clinical Laboratory Tests

| Laboratory | |
|---|---|
| Tests | Parameters |
| Hematology | Basophils (percentage and absolute count) |
| | Eosinophils (percentage and absolute count) |
| | Hematocrit |
| | Hemoglobin |
| | Lymphocytes (percentage and absolute count) |
| | Mean corpuscular hemoglobin |
| | Mean corpuscular hemoglobin concentration |
| | Mean corpuscular volume |
| | Monocytes (percentage and absolute count) |
| | Neutrophils (percentage and absolute count) |
| | Platelet count |
| | Red blood cell count |
| | Red blood cell distribution width |
| | White blood cell count |
| Clinical | Albumin |
| Chemistry | Alkaline phosphatase |
| | ALT |
| | AST |
| | Calcium |
| | Chloride |
| | Total cholesterol |
| | Creatinine |
| | Gamma glutamyl transferase |
| | Glomerular filtration rate (calculated using the |
| | CKD-EPI equation) |
| | Glucose (fasting [8 hours]) |
| | Lactate dehydrogenase |
| | Phosphorus |
| | Potassium |
| | Sodium |
| | Total bilirubin (if out of range, direct and indirect |
| | bilirubin are measured) |
| | Total protein |
| | Triglycerides |
| | Uric acid |
| Urinalysis | Bilirubin |
| | Blood |
| | Creatinine |
| | Glucose |
| | Ketones |
| | Leukocytes |
| | Nitrite |
| | pH and specific gravity |
| | Protein |
| | Urobilinogen |
| | Microscopic (only for abnormal urine stick test findings) |
| Serology | HBsAg |
| | Hepatitis C virus antibody |
| | HBcAb |
| | HIV ELISA |
| Urine drug | Amphetamines |
| screening test | Barbiturates |
| | Benzodiazepines |
| | Cannabinoids |
| | Cocaine |
| | Methylenedioxymethamphetamine |
| | Opiates |
| | Phencyclidine |
| | Propoxyphene |
| Pregnancy | Serum/urine β-hCG (WOCBP only) |
| testing | |
| Other | FSH (screening visit only, limited to female participants |
| | in whom nonchildbearing potential needs validation) |

### Results

The three-cohort study design described in the protocol above were expanded as follows:

The study has enrolled and dosed four sequential cohorts of 12 participants each (48 total, including 9 of Japanese descent). Participants were administered mAb 2419-1406 (200 mg/mL liquid) subcutaneously (SC). Doses for cohorts 1 through 4 were 200 mg (one 1 mL SC injection), 400 mg (two 1 mL SC injections), 400 mg (one 2 mL SC injection), and 600 mg (one 2 mL SC injection+one 1 mL SC injection), respectively. Participants received SC administered study drug on Day 1, were discharged from the institution on Day 2, and are being followed through day 112 on an outpatient basis. Standard safety assessments and blood sampling for PK and PD were performed at regular intervals.

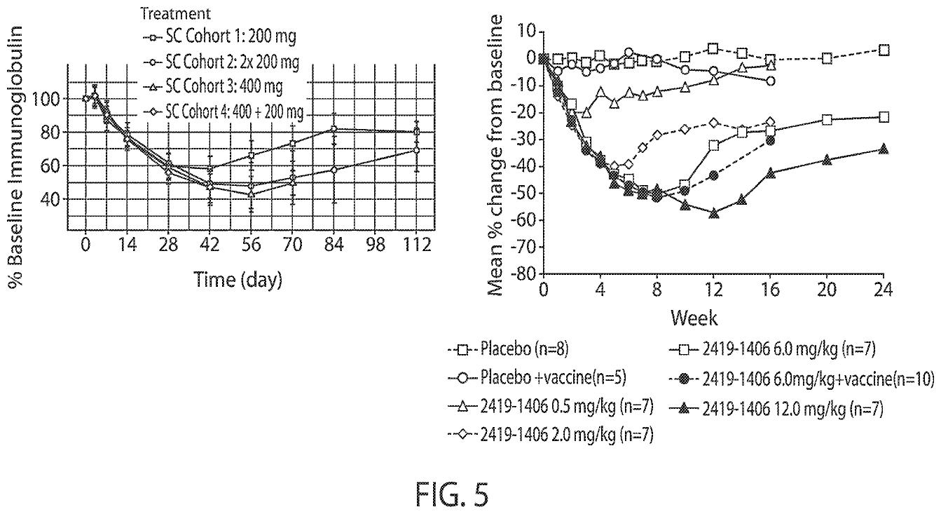

48 participants were enrolled and dosed with study drug. Cohorts 1 and 2 have completed the study visits. Cohorts 3 and 4 have completed dosing, and follow-up visits are currently ongoing. mAb 2419-1406 administered as a SC injection was well tolerated, with no serious adverse events (AEs) or AEs that led to study discontinuation, and no injection site reactions. Treatment Emergent AEs (TEAEs) were all mild and all resolved. There was no clinically relevant effect of treatment on laboratory tests, vital signs, electrocardiogram parameters, or physical examinations. Preliminary PK results showed bioavailability of approximately 75% in comparison to data from intravenous administration of mAb 2419-1406. Single SC doses of either 400 mg or 600 mg suppressed total IgA by up to approximately 60% from baseline values. This indicated a similar degree and trajectory of IgA suppression achieved by the intravenous (IV) formulation of mAb 2419-1406 in the first-in-human Phase 1 study in healthy volunteers (FIG. 5).

To summarize, this ongoing Ph1 study of single dose mAb 2419-1406 administered via SC route has, to date, been safe and well tolerated in healthy adults, demonstrated acceptable bioavailability, and suppressed total IgA by approximately 60% from baseline values at 400 mg and 600 mg doses. Final study data readout will likely support the further clinical development of SC-administered mAb 2419-1406 as a candidate treatment for IgAN.

### Example 3: Safety, Tolerability, Pharmacokinetics and Pharmacodynamics of Anti-APRIL Antibody Molecule in Healthy Volunteers

This example describes an in-human study to evaluate the safety and tolerability of an exemplary anti-APRIL antibody molecule, mAb 2419-1406, in healthy volunteers and to characterize the pharmacokinetics (PK) and pharmacodynamics (PD) of this antibody molecule.

Briefly, a Phase 1, randomized, double-blind, placebo-controlled, single ascending dose study of mAb 2419-1406 (in the form of humanized IgG2) was performed in healthy adult male and female volunteers. The study was conducted in sequential dosing cohorts. The first four cohorts (0.5, 2.0, 6.0, and 12.0 mg/kg, respectively) each enrolled 9 participants (4 of Japanese descent and 5 of non-Japanese descent) who were randomized to mAb 2419-1406 or placebo in a ratio of 7:2. In addition, a fifth cohort enrolled 15 adults randomized to receive 6.0 mg/kg mAb 2419-1406 or placebo (10:5), followed by tetanus/diphtheria vaccine challenge after 28 days (TENIVAC®, Sanofi Pasteur Limited; the effect of APRIL inhibition on vaccine response is described in a companion abstract). Participants received intravenous administration of 2419-1406 on Day 1, were discharged from the institution on Day 2, and were followed for 16 weeks on an outpatient basis. Standard safety assessments and blood sampling for PK and PD were performed at regular intervals.

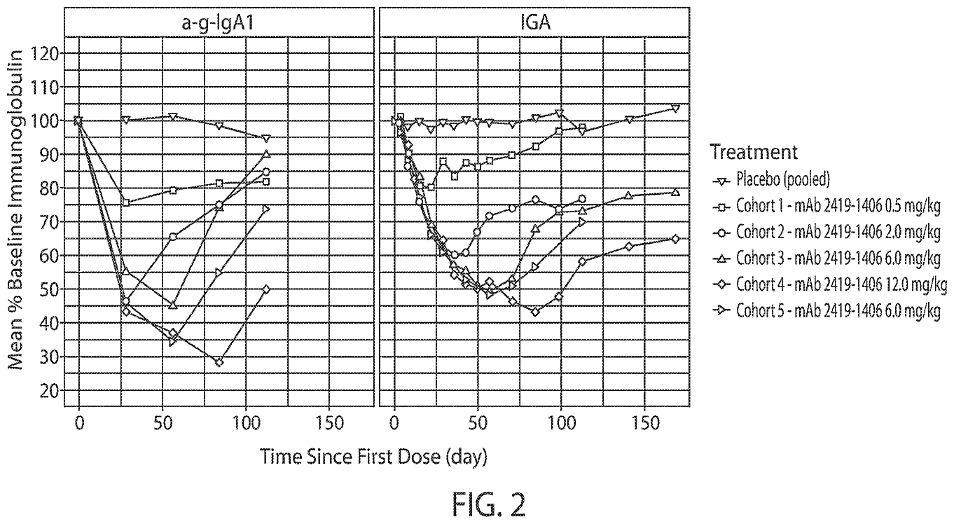

Overall, 51 participants were enrolled, randomized and dosed with mAb 2419-1406, of whom 47 (92.2%) completed the study. 2419-1406 was well tolerated, with no deaths, and no serious adverse events (AEs) or AEs that led to study discontinuation. Most treatment-emergent AEs (TEAEs) were mild; the incidence and severity of TEAEs were not dose dependent. One participant in the 2.0 mg/kg group experienced a severe TEAE of syncope following phlebotomy that the investigator considered unlikely to be related to study drug. There was no clinically relevant effect of treatment on laboratory tests, vital signs, electrocardiogram parameters, or physical examinations. Mab 2419-1406 had non-linear PK: half-life (t_{1/2}) increased with dose, while drug exposure (AUC) increased in a greater than dose proportional manner. Serum immunoglobulins (IgA, a-g IgA1, IgG, and IgM) were reversibly suppressed in a dose-dependent manner following mAb 2419-1406 administration. The mean comparison treatment, by immunoglobulin, is shown in FIG. 2. The maximum mean percentage reductions from baseline occurred at Week 12 for the 12.0 mg/kg dose: IgA, −57.2% (FIG. 2, right panel); a-g IgA1, −71.6% (FIG. 2, left panel); IgG, −33.6%; and IgM, −67.2%. These reductions were reversible, with a dose response in time to recovery. Mean free (non-mAb 2419-1406 bound) serum APRIL levels decreased to the lower limit of quantification (50 μg/mL) for all mAb 2419-1406 doses at Week 1, and also showed a dose response in time to recovery. No depletions in circulating lymphocyte populations were observed. There were no significant PK or PD differences between Japanese and non-Japanese participants.

In summary, mAb 2419-1406 single doses up to 12.0 mg/kg were safe and well tolerated in healthy adults. A single dose of mAb 2419-1406 was able to suppress free serum APRIL to the lower level of quantification. Serum a-g IgA1 decreased in parallel with total serum IgA and recovered in a dose-dependent manner following detection of free APRIL in serum.

### Example 4: Impact of Anti-APRIL Antibody Molecule on Tetanus- and Diphtheria-Toxoid Vaccination-Elicited Immune Responses in Healthy Volunteers

As shown in Example 3, an exemplary anti-APRIL antibody molecule, mAb 2419-1406, was associated with dose-dependent reductions in serum immunoglobulins (IgA, IgG and IgM), which were reversible and had a dose response in time to recovery. This example describes a study to examine whether APRIL suppression by mAb 2419-1406 influences the T-cell dependent antibody responses to tetanus and diphtheria toxoid vaccination was examined.

Briefly, a Phase 1, randomized, double-blind, placebo-controlled, single ascending dose study of mAb 2419-1406 (in the form of humanized IgG2) was conducted in healthy adult male and female volunteers. In one cohort within the study, participants were randomized in a 2:1 ratio to receive intravenous administration of 6.0 mg/kg mAb 2419-1406 or placebo, followed by a vaccine composed of tetanus and diphtheria toxoids (TENIVAC®, Sanofi Pasteur Limited), in order to evaluate the effect of mAb 2419-1406 on recipients' ability to generate a vaccine booster response. Participants received intravenous administration of mAb 2419-1406 on Day 1, were discharged from the institution on Day 2, received a single intramuscular dose of vaccine on Day 28, and were followed for 16 weeks thereafter on an outpatient basis. Blood samples were taken at regular intervals, and anti-tetanus toxoid and anti-diphtheria toxoid IgG, IgM and IgA quantitative ELISA assays were performed. Tetanus and diphtheria anti-toxoid IgG titers≥0.1 IU/mL were generally considered to be protective.

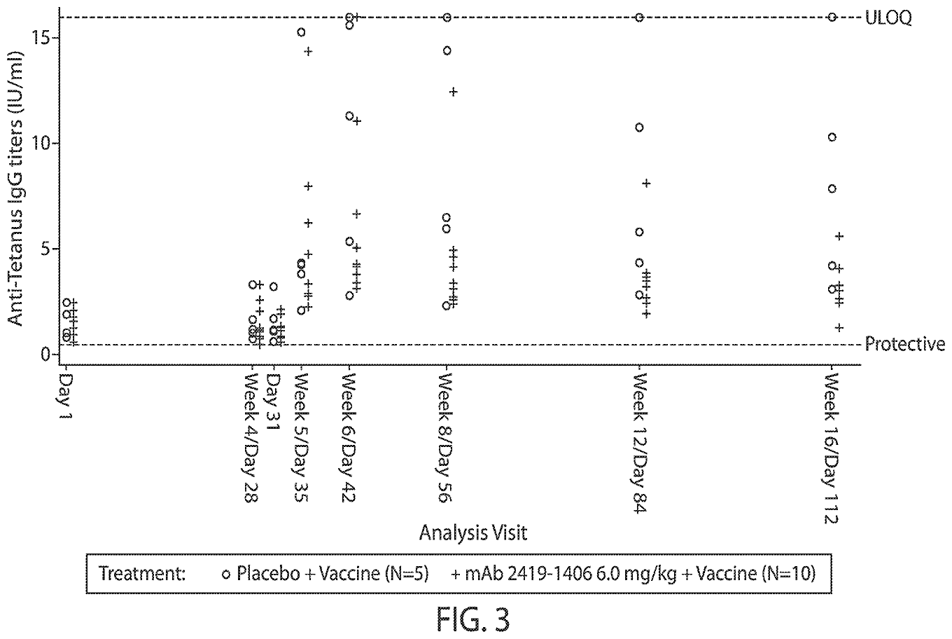

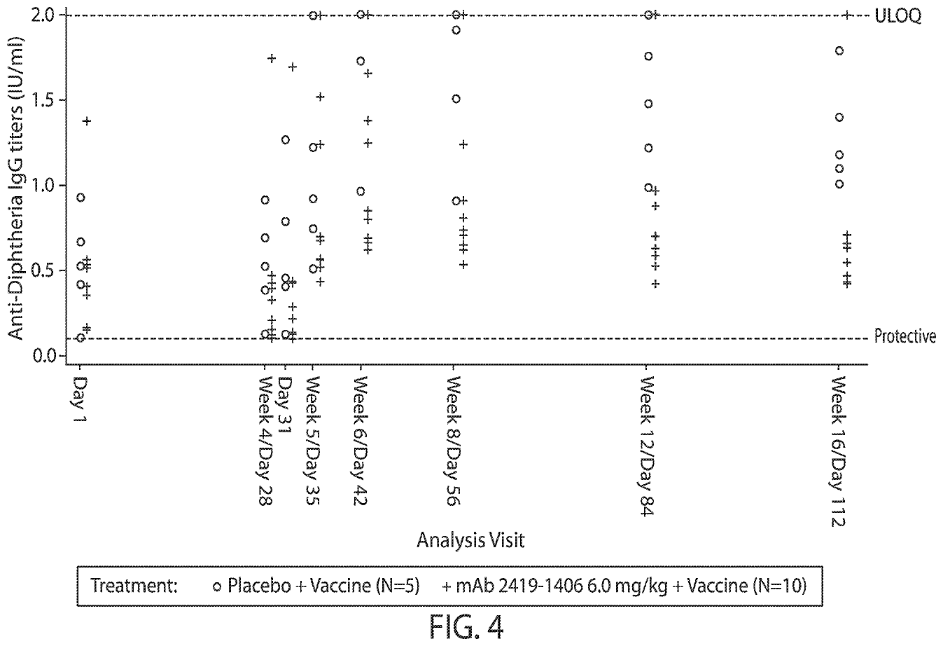

In the vaccination cohort, 15 participants were enrolled, randomized and dosed with mAb 2419-1406, of whom 14 completed the study, and one participant who received mAb 2419-1406 was lost to follow-up prior to receiving the vaccine. Both groups (placebo and mAb 2419-1406) demonstrated increased tetanus anti-toxoid IgG titers following immunization, with a mean 7.9-fold increase in IU/mL at Day 42 for placebo recipients and a mean 6.4-fold increase in IU/mL for mAb 2419-1406 recipients (FIG. 3). Tetanus anti-toxoid IgG titers greater than or equal to 0.1 IU/mL were considered protective. At visits after Day 42, tetanus anti-toxoid IgG titers declined faster in the mAb 2419-1406 group than in the placebo group (consistent with the reduction in total IgG associated with mAb 2419-1406 administration) but remained above the protective threshold of 0.1 IU/mL for all participants throughout the study (FIG. 3). Similar trends were observed for diphtheria anti-toxoid IgG titers, with a mean 5.5-fold increase in IU/mL at the Day 42 visit for placebo recipients and a mean 5.1-fold increase for mAb 2419-1406 recipients (FIG. 4). Diphtheria anti-toxoid IgG titers greater than or equal to 0.1 IU/mL were considered protective. There was no evidence of tetanus- or diphtheria-toxoid elicited IgM responses in either the placebo or mAb 2419-1406 groups, consistent with the recall nature of the vaccination. In a post hoc analysis, pre-existing serum anti-Td IgA titers fell between Day 1 and Day 28 consistent with the overall suppression of total serum IgA, were boosted to similar levels after vaccination in both groups and waned faster in the mAb 2419-1406 recipients thereafter.

In summary, mAb 2419-1406 treatment did not interfere with participants' ability to mount an antigen-specific serum IgG or IgA boost response to tetanus and diphtheria toxoid vaccination. There was no evidence of tetanus- or diphtheria-specific IgM responses in either the placebo or mAb 2419-1406 groups, consistent with recall vaccination exposure. These data indicate that qualitative T-cell dependent antibody responses are preserved during APRIL suppression.

### Example 5: Safety, Tolerability, Pharmacokinetics and Pharmacodynamics of Anti-APRIL Antibody in Healthy Volunteers

This example describes a Phase 1, first-in-human, randomized, double-blind, single ascending dose study aimed to evaluate the safety, pharmacokinetics and pharmacodynamics of an exemplary anti-APRIL antibody molecule, mAb 2419-1406 in healthy adults.

Participants were randomized to mAb 2419-1406 (a humanized IgG2 monoclonal antibody; sequential intravenous dosing cohorts: 0.5, 2.0, 6.0, 12.0 mg/kg) or placebo; a further cohort received MAB 2419-1406 6.0 mg/kg or placebo followed by a tetanus/diphtheria vaccine challenge.

Fifty-one participants were randomized, dosed, and analyzed for safety (7 for each MAB 2419-1406 dose; 8 for placebo; 10 for MAB 2419-1406+vaccine; 5 for placebo+vaccine). There were no serious adverse events or adverse events leading to study discontinuation. MAB 2419-1406 had non-linear pharmacokinetics: half-life increased with dose, and drug exposure increased in a greater than dose-proportional manner. Serum APRIL, IgA, galactose-deficient IgA1, IgG, and IgM were reversibly suppressed in a dose-dependent manner, with a dose-response in time to recovery. Tetanus and diphtheria serum IgG titers increased following recall vaccination.

MAB 2419-1406 was safe, well tolerated, and reversibly suppressed APRIL and various immunoglobulins, without loss of antigen-specific vaccination response. Further clinical development of MAB 2419-1406 for IgA nephropathy is warranted.

### INTRODUCTION

The primary objective of the present first-in-human study was to evaluate the safety and tolerability of mAb 2419-1406 in healthy volunteers. Secondary objectives included characterization of the pharmacokinetics (PK) and pharmacodynamics (PD) of mAb 2419-1406. Exploratory objectives included investigating whether mAb 2419-1406 suppression of APRIL influences antibody responses to tetanus and diphtheria toxoid vaccination.

### Methods

Study Design and Participants

Participants (male or non-pregnant female) were eligible for the study if they were aged 18-55 years, had a body mass index of 18-32 kg/m², were healthy as judged by the principal investigator based on medical evaluation and laboratory tests, and had serum IgG>750 mg/dl, serum immunoglobulin M (IgM)>55 mg/dl, and serum IgA>80 mg/dl. The study enrolled participants of Japanese descent and non-Japanese descent, as established by verbal confirmation (all 4 grandparents were born in Japan or all 4 grandparents were non-Japanese, respectively). Patients were excluded if they had a history or presence of a serious medical condition, including a mental disorder; a history or presence of proteinuria, chronic kidney disease, or were considered to be immunosuppressed; had previously received an antibody or biologic therapy within 30 d or 5 half-lives; had a history of severe allergic reaction or hypersensitivity reaction to tetanus/diphtheria toxoid-containing vaccine; had received a tetanus vaccine in the past 5 years; had known hypoglobulinemia disorder; had a pre-existing latent infection, infection requiring hospitalization or treatment with antivirals or antibiotics, or vaccination within 30 d; were concomitantly using systemic immunosuppressive or immunomodulatory medications; or had a positive urine drug, alcohol or cotinine test.

The study consisted of a≤28-d screening period, an in-house stay of 2-3 d (admission to the study center on Day −1 [baseline], randomization and dosing on Day 1, and discharge on Day 2), and a 16-24-week follow-up period with frequent outpatient visits. The study was conducted in sequential dose escalating cohorts. The first four cohorts (0.5, 2.0, 6.0, and 12.0 mg/kg, respectively) each enrolled 9 participants who were randomized to mAb 2419-1406 or placebo in a 7:2 ratio. In addition, a fifth cohort (cohort 5) enrolled 15 adults randomized in a 2:1 ratio to receive mAb 2419-1406 6.0 mg/kg or placebo on Day 1, followed by a tetanus and diphtheria toxoid vaccine (TENIVAC®, Sanofi Pasteur Limited) on Day 28 to evaluate the effect of APRIL suppression by mAb 2419-1406 on recipients' ability to generate a response to routine vaccine boost.

mAb 2419-1406 doses were calculated on a per participant weight basis and diluted in 100 ml of 0.9% sodium chloride; placebo (manufactured by Baxter Corporation, Deerfield, IL) consisted of 0.9% sodium chloride. mAb 2419-1406 and placebo were administered intravenously using a volumetric pump through a 0.22 m inline intravenous filter over the course of 1 h, in the morning after a light meal. In cohorts 1-4, 2 participants (one receiving mAb 2419-1406 and one receiving placebo) were dosed at least 24 h before the others, in case of unanticipated adverse reactions. Cohorts 1-4 were stratified to include 4 participants of Japanese descent and 5 of non-Japanese descent; no more than 1 Japanese participant per cohort was randomized to receive placebo.

Concomitant medication use was not permitted in the period from 30 d (or 5 half-lives, if longer) preceding baseline until the end of the study, except for acetaminophen, ibuprofen, hormonal contraceptives, topical medications, vitamins, and dietary or herbal remedies. Medication for the treatment of adverse events (AEs) was permitted if agreed by the principal investigator and medical monitor.

Blood samples for PK were collected on Days 1 (pre- and post-dose), 2, and 3, and at the Week 1, 2, 4, 6, 8, 10, and 16 visits. Serum samples were analyzed for mAb 2419-1406 concentrations by Syneos Health (Princeton, NJ) using a validated electrochemiluminescence (ECL) immunoassay: mAb 2419-1406 was captured on a Meso Scale Discovery (MSD) plate coated with mouse anti-mAb 2419-1406 antibody; ruthenylated anti-mAb 2419-1406 and MSD read buffer were added; then plates were read using an MSD ECL plate reader.

Blood samples to measure immunoglobulin levels were collected at baseline, Day 3, and at the Week 1, 2, 3, 4, 5, 6, 7, 8, 10, 12, 14 and 16 visits, and also on additional visits at Week 20 and 24 for participants in cohorts 3 and 4. Serum samples were analyzed for total IgA, IgG, and IgM by GenX Laboratory (Los Angeles, CA) using an AU480 Chemistry Analyzer (Beckman Coulter, Brea, CA). Serum Gd-IgA₁ was analyzed using a validated solid-phase sandwich enzyme-linked immunosorbent assay (ELISA) with the #27600 Gd-IgA₁ Assay Kit (KM55 anti-Gd-IgA₁) (Immuno-Biological Laboratories Co., Ltd., Fujioka-Shi, Gunma, Japan) by Syneos Health. Serum APRIL was analyzed using a validated APRIL ECL immunoassay by Syneos Health: APRIL was captured on an MSD plate coated with anti-recombinant human (rh)APRIL; biotinylated anti-rhAPRIL, SULFO-TAG streptavidin (detection reagent), and MSD read buffer were added; then plates were read using an MSD ECL plate reader. Serum B cell activating factor (BAFF) was analyzed post hoc using human premixed Luminex assay kits (R&D Systems, Minneapolis, MN) and a MAGPIX® (Luminex, Austin, TX), as per the manufacturer's instructions.

Tetanus/diphtheria serology were examined in cohort 5, and anti-tetanus toxoid and anti-diphtheria quantitative ELISA assays were performed by Q2 Solutions LLC (Valencia, CA) (IgG) and the University of Maryland Center for Vaccine Development and Global Health (Baltimore, MD) (IgM and IgA) at Week 4 (pre-vaccination), Day 31, and at the Week 5, 6, 8, 12, and 16 visits. In ELISA assays, tetanus and diphtheria anti-toxoid IgG titers≥0.1 IU/ml are considered to be protective.

### Statistical Analyses

Safety outcomes were summarized using descriptive statistics in the safety sample, defined as all randomized participants who received at least 1 dose of study drug. PK outcomes were summarized using descriptive statistics in the PK sample, defined as all randomized participants with at least one quantifiable mAb 2419-1406 concentration. Values below the lower limit of quantification (LLQ; 0.1 μg/ml for mAb 2419-1406) were imputed as zero, with the exception of the mAb 2419-1406 concentration-time figure, which used a log axis (imputed as the LLQ). PD outcomes were summarized using descriptive statistics in the PD sample, defined as the safety sample subset with at least one PD parameter assessment (IgA, IgG, IgM) post-study-drug dosing. Values below the LLQ (0.5 μg/ml for Gd-IgA₁, 50 μg/ml for APRIL, 0.1 IU/ml for diphtheria IgG) were imputed as the LLQ, and values above the upper limit of quantification (ULQ; 16.0 IU/ml for tetanus IgG, 2.00 IU/ml for diphtheria IgG) were imputed as the ULQ.

Statistical analyses were performed using SAS® Version 9.3 (SAS Institute Inc., Cary, NC) and Phoenix® WinNonlin® Version 8.0 or higher (Certara USA, Inc., Princeton, NJ).

### Results

### Participants

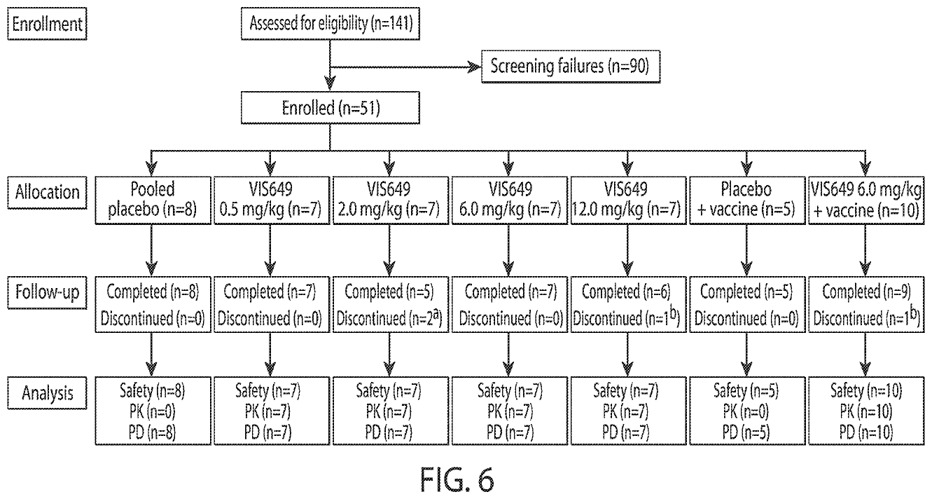

Overall, 51 participants were enrolled, randomized and dosed with study drug, of whom 47 (92.2%) completed the study (FIG. 6). In cohort 5, 14 out of 15 participants received the vaccine; 1 participant in the mAb 2419-1406+vaccine group was lost to follow-up and did not receive the vaccine. Baseline demographic characteristics were generally similar across treatment groups. Cohort 5 (participants who also received vaccine) had a different ethnic mix as there was no requirement for Japanese/non-Japanese descent in this cohort.

### Safety and Tolerability

mAb 2419-1406 was well tolerated, with no serious AEs or AEs that led to study discontinuation. Treatment-emergent AEs (TEAEs) were experienced by 4/8 (50.0%) participants who received placebo, 11/28 (39.3%) who received mAb 2419-1406 (all doses), ⅗ (60.0%) who received placebo+vaccine, and 4/10 (40.0%) who received mAb 2419-1406+vaccine (TABLE 15). Across all cohorts, there was a numerically greater incidence of upper respiratory tract infection among participants receiving placebo ( 2/13; 15.4%) compared with those receiving mAb 2419-1406 (all doses; 2/38; 5.3%). The incidence of TEAEs was not dose dependent.

Most TEAEs were mild. Two moderate TEAEs were reported: back pain by 1 participant who received 6.0 mg/kg (considered not related to study drug), and vomiting and migraine by 1 participant who received 6.0 mg/kg (considered unlikely to be related to study drug). One participant in the 2.0 mg/kg group experienced a severe TEAE of vasovagal syncope following phlebotomy on Day 29 after dosing (considered unlikely to be related to study drug). The severity of TEAEs was not dose dependent, and all TEAEs had resolved or were resolving by the end of the study.

No meaningful differences were noted between the Japanese and non-Japanese participants with regard to TEAEs.
There was no clinically relevant effect of treatment on laboratory tests, vital signs, electrocardiogram parameters, or physical examinations.

TABLE 15
Treatment-emergent adverse events for the clinical trial participants. TEAE, treatment-
emergent adverse event; URTI, upper respiratory tract infection.

| | | | | | mAb | | mAb |
|---|---|---|---|---|---|---|---|
| | | mAb | mAb | mAb | 2419-1406 | | 2419-1406 |
| | Pooled | 2419-1406 | 2419-1406 | 2419-1406 | 12.0 | Placebo + | 6.0 mg/kg + |
| | placebo | 0.5 mg/kg | 2.0 mg/kg | 6.0 mg/kg | mg/kg | vaccine | vaccine |
| | (n = 8) | (n = 7) | (n = 7) | (n = 7) | (n = 7) | (n = 5) | (n = 10) |
| At least 1 | 4 (50.0) | 2 (28.6) | 3 (42.9) | 3 (42.9) | 3 (42.9) | 3 (60.0) | 4 (40.0) |
| TEAE | | | | | | | |

TEAEs occurring in >1 participants who received mAb 2419-1406 (across mAb 2419-1406 groups)

| | | | | | | | |
|---|---|---|---|---|---|---|---|
| URTI | 1 (12.5) | 0 | 1 (14.3) | 1 (14.3) | 0 | 1 (20.0) | 0 |
| Diarrhea | 1 (12.5) | 0 | 1 (14.3) | 0 | 1 (14.3) | 0 | 0 |
| Dizziness | 0 | 0 | 0 | 1 (14.3) | 1 (14.3) | 0 | 0 |

TEAE, treatment-emergent adverse event; URTI, upper respiratory tract infection.
Data are presented as n (%).

### Pharmacokinetics

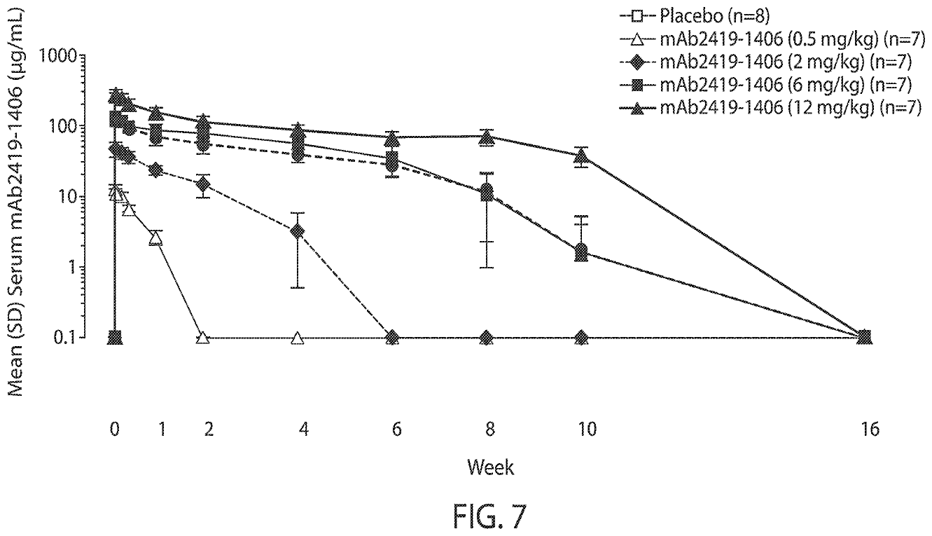

Concentration-time profiles indicated that mAb 2419-1406 pharmacokinetics were non-linear following a single dose (FIG. 7). mAb 2419-1406 exposure increased in a greater than dose-proportional manner. At the higher tested doses (6.0 and 12.0 mg/kg), serum mAb 2419-1406 concentrations were biexponential, with a rapid distribution phase followed by an (initially) slower elimination phase. Across all doses, the rate of mAb 2419-1406 elimination increased at concentrations below approximately 50 μg/ml.

Maximum serum concentration (Cₘₐₓ) increased in an approximately dose-proportional manner (TABLE 16). Other PK parameters indicated non-linear PK: half-life (t,) increased with dose, drug exposure (AUC) increased in a greater than dose-proportional manner, and total clearance (CL) decreased with dose. Apparent volume of distribution was not dose dependent.

Administration of the vaccine had no noteworthy effects on mAb 2419-1406 PK (TABLE 16), and there were no significant PK differences between Japanese and non-Japanese participants.

TABLE 16
Mean pharmacokinetic parameters of mAb 2419-1406 following a single intraveneous
dose (pharmacokinetics sample) AUC_{0-W 16/0-∞}, area under the concentration-
time curve from pre-dose (time 0) to Week 16/infinity; CL, apparent clearance;
Cₘₐₓ, maximum serum concentration; t_{1/2}, terminal elimination half-life;
Vd, apparent volume of distribution.

| | | | | mAb 2419- | |
|---|---|---|---|---|---|
| | mAb 2419- | mAb 2419- | mAb 2419- | 1406 | mAb 2419- |
| | 1406 | 1406 | 1406 | 6.0 mg/kg + | 1406 |
| | 0.5 mg/kg | 2.0 mg/kg | 6.0 mg/kg | vaccine | 12.0 mg/kg |
| | (n = 7) | (n = 7) | (n = 7) | (n = 10) | (n = 7) |
| Cₘₐₓ (μg/ml) | 12.6 | 48.6 | 136 | 131 | 283 |
| AUC_{0-W 16} | 1120 | 11700 | 74500 | 57900 | 176000 |
| (h · μg/ml) | | | | | |
| AUC_{0-∞} (h · μg/ml) | 1120 | 11700 | 74400 | 57900 | 187000 |
| t_{1/2} (h) | 60.7 | 175 | 231 | 342 | 670 |
| CL (ml/h) | 31.5 | 11.7 | 6.13 | 8.72 | 4.51 |
| Vd (ml) | 2750 | 2850 | 2180 | 3910 | 4120 |

AUC_{0-W 16/0-∞}, area under the concentration-time curve from pre-dose (time 0) to Week 16/infinity; CL, apparent clearance; Cₘₐₓ, maximum serum concentration; t_{1/2}, terminal elimination half-life; Vd, apparent volume of distribution.

### Pharmacodynamics

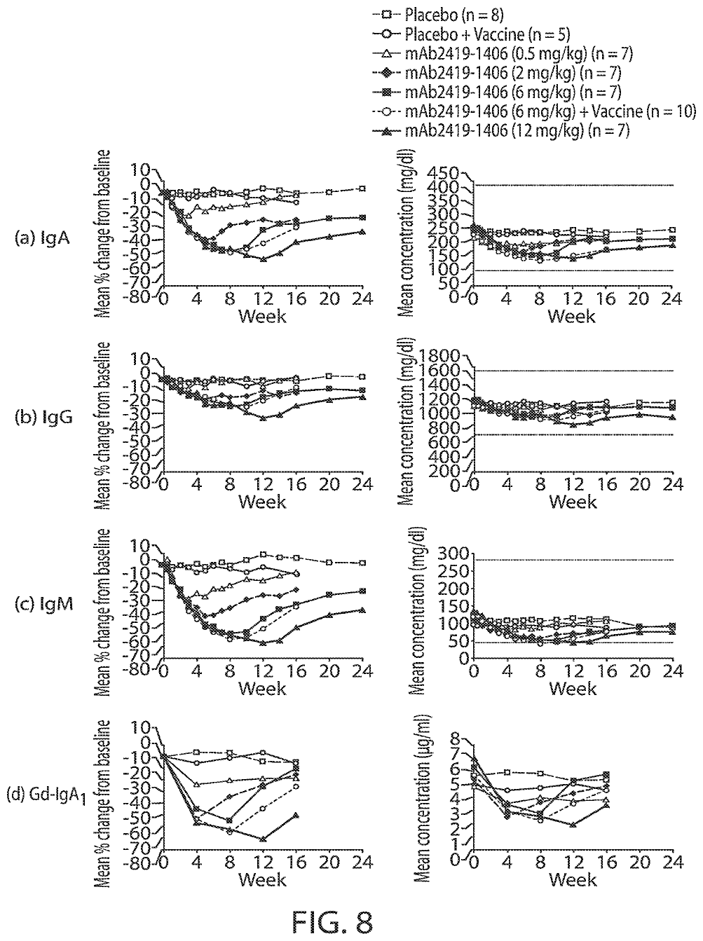

Serum immunoglobulins (IgA, IgG, IgM, and Gd-IgA₁) were suppressed in a dose-dependent manner following mAb 2419-1406 administration (FIG. 8). Maximum mean percentage reductions from baseline were observed at the Week 12 time point with the 12.0 mg/kg dose: IgA, −57.2%; IgG, −33.6%; IgM, −67.2%; and Gd-IgA₁, −71.6%. These reductions appeared to be reversible, with a dose response in time to recovery.

No mean or median values fell below the normal ranges for IgA or IgG; for IgM only, several mean and/or median values fell slightly below the normal range (i.e., <45 mg/dl) between Weeks 5 and 14 in the mAb 2419-1406 groups, but all values recovered and no AEs related to low IgM levels were recorded.

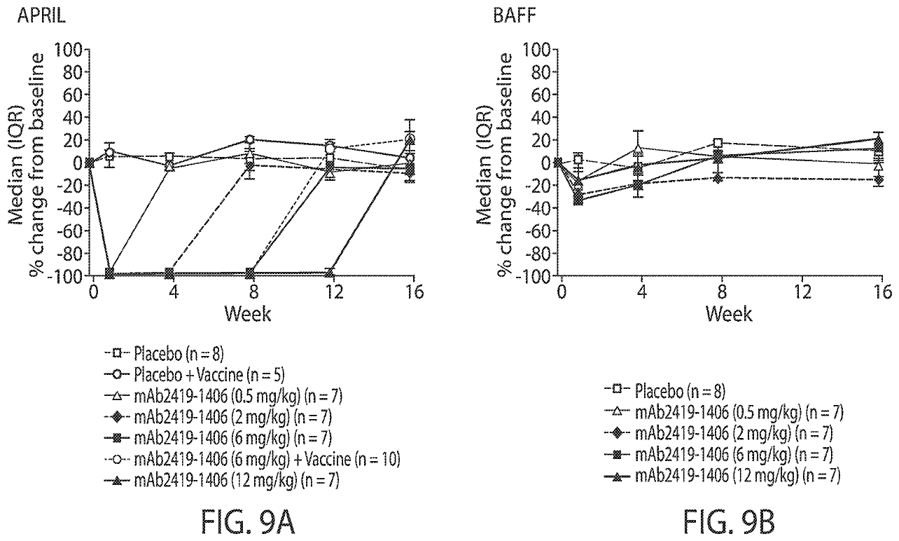

Mean serum free (non-mAb 2419-1406 bound) APRIL levels decreased from baseline to at or below the LLQ (50 μg/ml) for all mAb 2419-1406 doses at Week 1 and showed a dose response in time to recovery. Recovery to pre-dose levels was observed by Week 4 for the 0.5 mg/kg group, Week 8 for the 2.0 mg/kg group, Week 12 for the 6.0 mg/kg group, and Week 16 for the 12.0 mg/kg group (FIG. 9A).

Serum BAFF showed minimal change from baseline, with median changes<35% at all time points for all doses studied (FIG. 9B).

No notable depletions in circulating lymphocyte populations (B cell, T cell, or natural killer [NK]cell types) were observed after mAb 2419-1406 dosing at any of the dose levels, or with placebo (TABLE 17).

There were no notable PD differences between Japanese and non-Japanese participants.

TABLE 17
Summary of lymphocyte subsets following a single intravenous dose of mAb 2419-1406
(pharmacodynamics sample). CD, cluster of differentiation; NK, natural killer. Data are presented
as mean (standard deviation). ^{a}n = 7; ^{b}n = 5; ^{c}n = 6.

| | | | mAb | mAb | mAb | mAb |
|---|---|---|---|---|---|---|
| | | Pooled | 2419-1406 | 2419-1406 | 2419-1406 | 2419-1406 |
| | | placebo | 0.5 mg/kg | 2.0 mg/kg | 6.0 mg/kg | 12.0 mg/kg |
| | | (n = 8) | (n = 7) | (n = 7) | (n = 7) | (n = 7) |
| CD19 | Baseline | 205.4 (128.3) | 194.6 (23.7) | 225.7 (89.7) | 262.6 (80.0) | 175.9 (49.4) |
| (B cells) | Change at | −21.1 (97.3)^{a} | 25.1 (56.1) | −5.4 (26.5)^{b} | 84.6 (81.8) | 23.7 (24.6) |
| (cells/μl) | Week 16 | | | | | |
| CD20+ | Baseline | 209.4 (154.1) | 212.1 (47.6)^{b} | 221.4 (95.7) | 273.2 (66.2)^{c} | 173.0 (66.4) |
| (B cells) | Change at | −19.8 (134.8)^{a} | 12.2 (73.0)^{b} | −5.6 (50.4)^{b} | 133.1 (139.8)^{c} | 16.1 (16.3) |
| (cells/μl) | Week 16 | | | | | |
| CD4 | Baseline | 659.8 (178.0) | 633.6 (156.9) | 803.7 (145.8) | 579.3 (288.7) | 716.1 (166.1) |
| (T cells) | Change at | −14.9 (175.5)^{a} | 14.0 (161.1) | −75.4 (80.7)^{b} | 121.6 (149.2) | 135.6 (204.7) |
| (cells/μl) | Week 16 | | | | | |
| CD8 | Baseline | 432.3 (145.1) | 463.7 (165.3) | 395.0 (103.2) | 332.4 (108.1) | 362.6 (199.7) |
| (T cells) | Change at | −14.0 (113.7)^{a} | -14.9 (135.7) | −49.0 (68.1)^{b} | 86.0 (94.3) | 94.3 (96.8) |
| (cells/μl) | Week 16 | | | | | |
| CD16 + CD56 | Baseline | 175.3 (156.1) | 147.3 (38.3) | 181.9 (88.4) | 113.4 (40.8) | 134.0 (48.3) |
| (NK cells) | Change at | 0.4 (46.2)^{a} | 13.9 (49.9) | 20.2 (91.4)^{b} | 34.9 (51.4) | 15.7 (41.1) |
| (cells/νl) | Week 16 | | | | | |

CD, cluster of differentiation; NK, natural killer.
Data are presented as mean (standard deviation).
^{a}n = 7; ^{b}n = 5; ^{c}n = 6.

### Vaccine Response

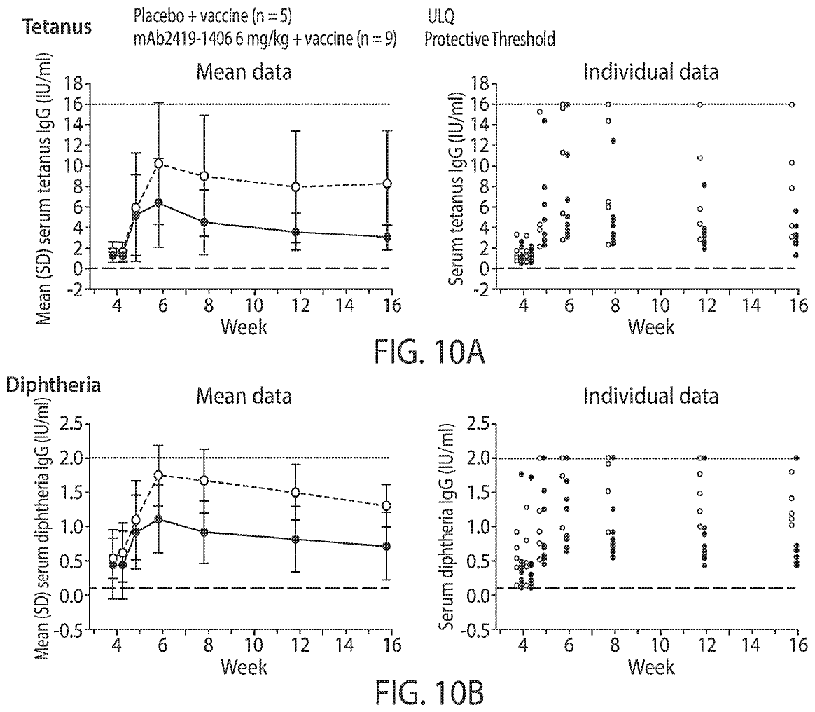

Both groups (placebo and mAb 2419-1406 6.0 mg/kg) demonstrated increased tetanus toxoid IgG titers following immunization, with a maximum mean increase at Week 6 (FIG. 10A). At Week 6 relative to pre-vaccination (Week 4) values, a mean 7.9-fold increase in antibody titers was observed in the placebo group and a mean 6.4-fold increase was observed in the mAb 2419-1406 group. Tetanus toxoid IgG titers declined from Week 6 onwards. Antibody levels in the mAb 2419-1406 group were lower compared with the placebo group (consistent with the reduction in total IgG associated with mAb 2419-1406 administration), but remained above the protective threshold of 0.1 IU/ml for all participants throughout the study (FIG. 10A).

Diphtheria IgG titers were higher in the placebo group than in the mAb 2419-1406 group prior to immunization (Week 4), which translated into higher titers in the placebo group following immunization (FIG. 10B). However, titer fold-change responses were similar between groups following immunization, with a mean 5.5-fold increase in concentration for placebo recipients and a mean 5.1-fold increase for mAb 2419-1406 recipients at the Week 6 visit. Diphtheria IgG titers declined from Week 6 onwards, with lower values in the mAb 2419-1406 group compared with the placebo group (consistent with the reduction in total IgG associated with mAb 2419-1406 administration). All participants maintained titers>0.1 IU/ml throughout the study, except for one participant in the mAb 2419-1406 group who had titers of <0.10 IU/ml (the LLQ) at Week 4 (pre-vaccination) and Day 31 only (FIG. 10B).

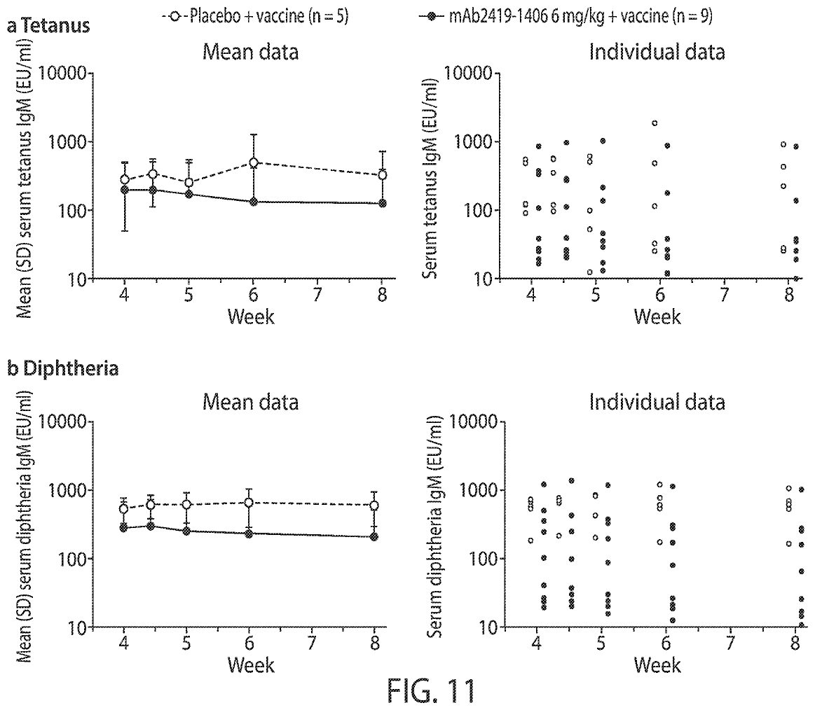

There was no evidence of tetanus- or diphtheria-specific IgM responses in either the placebo or VIS649 groups, consistent with the recall nature of the vaccination (FIG. 11).

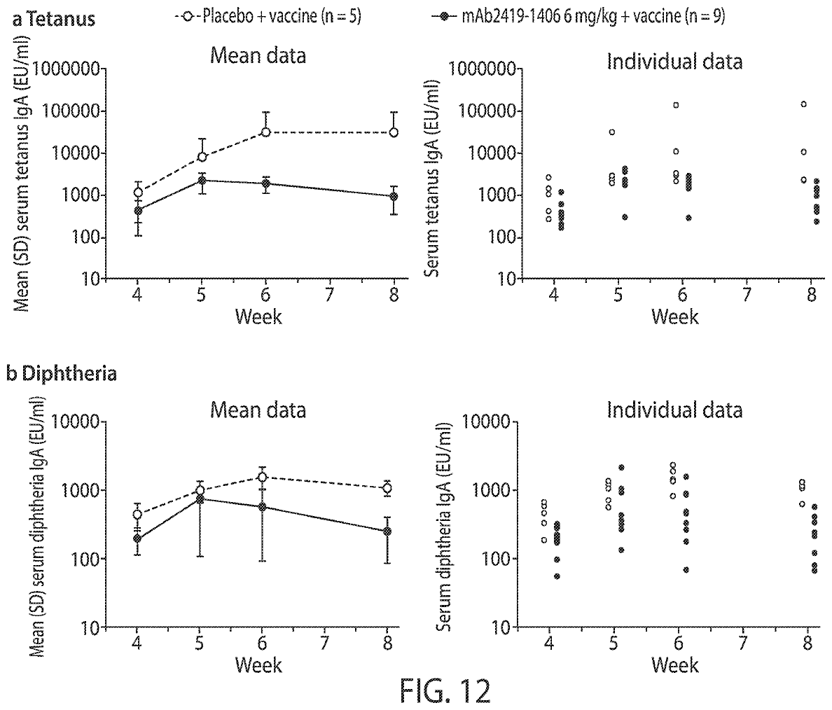

In a post hoc analysis, pre-existing serum tetanus/diphtheria anti-toxoid IgA titers fell between Day 1 and Week 4 in the mAb 2419-1406 group (consistent with the overall suppression of total serum IgA), were boosted after vaccination in both groups (with peak recall response>6-fold for tetanus and >4-fold for diphtheria relative to pre-vaccination values [i.e., Week 4]), and declined faster in mAb 2419-1406 recipients thereafter (FIG. 12).

### DISCUSSION

In this study, a single dose of mAb 2419-1406 (up to 12.0 mg/kg) suppressed serum free APRIL to at or below the LLQ at the first post-dose time point (Week 1). Serum IgA and Gd-IgA₁ decreased in parallel with serum APRIL, and recovered in a dose-dependent manner following reappearance of free APRIL in serum over the following weeks. Serum IgM followed a similar pattern.

Whereas serum free APRIL was suppressed, serum BAFF—which shares receptors with APRIL—was minimally affected. This indicates that VIS649 selectively inhibits APRIL, with intact BAFF signaling,

mAb 2419-1406 treatment did not interfere with participants' ability to mount a substantial antigen-specific serum IgG or IgA booster response to tetanus and diphtheria toxoid vaccination (with fold increases in IgG titers similar to those of placebo recipients), despite an overall reduction in the respective immunoglobulins. There was no evidence of tetanus- or diphtheria-specific IgM responses in either the placebo or mAb 2419-1406 groups 4 weeks post-vaccination, consistent with recall response. These data indicate that qualitative antibody responses to routine vaccine antigens are preserved during APRIL suppression. Overall, mAb 2419-1406 appears to have the ability to suppress pathogenic Gd-IgA₁ (and other immunoglobulins) while allowing for a booster immune response to tetanus and diphtheria vaccines.

With regard to PK, mAb 2419-1406 elimination became more rapid at concentrations below approximately 50 μg/ml. mAb 2419-1406 t, increased with dose, AUC increased in a greater than dose-proportional manner, and CL decreased with dose, indicating non-linear PK in this single-dose range. Regardless of this non-linearity, mAb 2419-1406 suppressed APRIL and serum immunoglobulins at all studied doses.

There were no safety concerns with a single dose of mAb 2419-1406 in healthy participants. Notably, there was no increased risk of respiratory tract infections or other infections versus placebo, and no indication of infusion reactions, delayed allergic reactions, or intravenous infusion site reactions.

### CONCLUSION

Through its anti-APRIL effects, mAb 2419-1406 specifically targets a key underlying disease mechanism in IgAN (i.e., production of Gd-IgA₁), and, together with its therapeutically relevant PK/PD parameters and favorable safety profile, these data support the further clinical development of VIS649 as a potential targeted treatment for IgAN.

### Example 6. Assessment of Pharmacokinetic Properties of mAb 2419-1406

This Example describes an in-human study to characterize the pharmacokinetic properties of mAb 2419-1406 in the one month following administration.

In this example, IgA nephropathy patients were administered a single, weight-adjusted intravenous dose of 2 mg/kg, 4 mg/kg, or 8 mg/kg of mAb 2419-1406, and blood samples were collected at 1, 3, 8, 18, or 30 days post-administration. In brief, levels of mAb 2419-1406 in the serum were determined using an anti-idiotype antibody in an ELISA format assay. Post-baseline below limit of quantification (BLQ) values were set to 0. As shown in FIGS. 13A and 13B, dose-dependent reductions in serum mAb 2419-1406 levels overtime were observed. For all dose groups, declines in serum levels of mAb 2419-1406 were observed between 1 to 8 days post-administration, while slower declines were observed between the day 8 and day 30 time points. Across all time points, the highest mAb 2419-1406 levels were detected in patients that received the 8 mg/kg dose, with approximately 50 μg/mL of mAb 2419-1406 detected in the serum of the 8 mg/kg dose group at 30 days post-administration.

### Example 7. Effects of mAb 2419-1406 on Serum IgA, IgG, and IgM Levels

This Example describes suppression of serum IgA, IgG, and IgM levels of human IgA nephropathy patients following administration of mAb 2419-1406.

In this example, IgA nephropathy patients were randomized to receive a weight-adjusted dose of placebo, 2 mg/kg, 4 mg/kg, or 8 mg/kg of mAb 2419-1406 once a month. Blood samples were collected at multiple time points, with emphasis at later time points for sample collection immediately prior to administration of mAb 2419-1406. Total IgA, IgG, and IgM concentrations in the serum were determined using IgA-, IgG-, or IgM-selective ELISA format assays, respectively. Relative levels of IgA, IgG, and IgM were calculated as percentages compared to baseline levels, and baseline levels were measured at the month 0 day 1 time point prior to administration of mAb 2419-1406. The time points and number of samples collected are indicated in Table 18 and 19. Later time point data were masked to avoid the risk of unblinding.

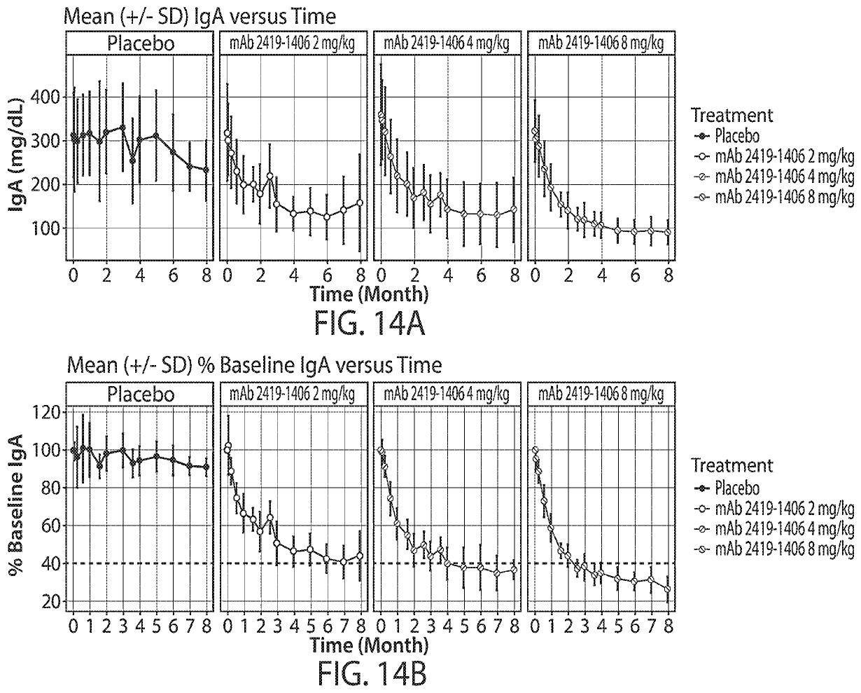

As shown in FIG. 14A and Table 18, levels of serum IgA decreased in a dose-dependent manner following mAb 2419-1406 dosing. When levels of serum IgA were compared to baseline levels, patients receiving placebo exhibited minimal changes in serum IgA levels, with a mean of 91.2% of baseline serum IgA levels observed at the month 8 time point (FIG. 14B, Table 19). In contrast, dose-dependent reductions in IgA levels were observed by 18 days after the first mAb 2419-1406 dosing, with the mean serum levels of IgA ranging from 74.9% for the 2 mg/kg group to 73.0% for the 8 mg/kg group compared to baseline (FIG. 14B, Table 19). These reductions increased over time, and at the month 8 time point, the mean serum IgA levels compared to baseline were 44.3% for the 2 mg/kg group, 36.9% for the 4 mg/kg group, and 26.6% for the 8 mg/kg group (FIG. 14B, Table 19). Across all time points assayed, patients in the 8 mg/kg dose group exhibited the greatest reductions in serum IgG levels compared to the other dose groups, and patients in the 4 mg/kg dose group exhibited greater reductions in serum IgG levels as compared to the 2 mg/kg dose group (FIG. 14B, Table 19).

Administration of mAb 2419-1406 led to an increase in the percentage of patients that achieved>60% reduction in IgA levels from baseline, with a dose-dependent response observed (Table 20). None of the patients receiving placebo achieved>60% reduction in IgA levels over the course of the study (Table 20). In contrast, patients achieved>60% reduction in IgA levels following administration of mAb 2419-1406, with at least one patient from every mAb 2419-1406 dose group achieving this reduction by the month 2 time point (Table 20). At the month 8 time point, ⅓ (33.3%), ⅚ (83.3%), and 5/5 (100%) of the patients who received the 2 mg/kg, 4 mg/kg, and 8 mg/kg mAb 2419-1406 dose, respectively, exhibited a>60% reduction in IgA levels compared to baseline (Table 20).

When assessing the number of patients that achieved>40% reduction in IgA levels from baseline, none of the patients receiving placebo achieved this target across all time points (Table 21). Administration of mAb 2419-1406 resulted in a greater percentage of patients achieving>40% reduction in IgA levels, with 100% of assessed patients from all dose groups achieving the target reduction by approximately the month 4 time point (Table 21).

TABLE 18
Concentration of IgA in Serum by Time and Dose following Administration of mAb 2419-1406

| | IgA (mg/dL) Mean ± SD, n | | | | | | | |
|---|---|---|---|---|---|---|---|---|
| | Month 0 | Month 0 | Month 0 | Month 0 | | Month 1 | | Month 2 |
| Arm | Day 1 | Day 3 | Day 8 | Day 18 | Month 1 | Day 18 | Month 2 | Day 18 |
| Placebo | 314 ± 97, | 303 ± 120, | 300 ± 96, | 313 ± 93, | 317 ± 97, | 299 ± 137, | 320 ± 96, | NA |
| | n = 27 | n = 7 | n = 26 | n = 25 | n = 26 | n = 4 | n = 22 | |
| 2 mg/kg | 319 ± 112, | 303 ± 82, | 271 ± 82, | 229 ± 74, | 199 ± 66, | 200 ± 40, | 178 ± 68, | 219 ± 73, |
| | n = 25 | n = 5 | n = 24 | n = 24 | n = 23 | n = 4 | n = 19 | n = 3 |
| 4 mg/kg | 359 ± 116, | 348 ± 92, | 321 ± 101, | 264 ± 85, | 219 ± 82, | 202 ± 73, | 168 ± 69, | 181 ± 62, |
| | n = 25 | n = 7 | n = 25 | n = 24 | n = 24 | n = 8 | n = 22 | n = 6 |
| 8 mg/kg | 323 ± 71, | 305 ± 47, | 288 ± 71, | 236 ± 64, | 194 ± 53, | 155 ± 28, | 140 ± 41, | 121 ± 26, |
| | n = 25 | n = 6 | n = 25 | n = 25 | n = 23 | n = 5 | n = 21 | n = 6 |
| | | IgA (mg/dL) Mean ± SD, n | | | | | | |
| | | | Month 3 | | | | | |
| | Arm | Month 3 | Day 18 | Month 4 | Month 5 | Month 6 | Month 7 | Month 8 |
| | Placebo | 331 ± 101, | 253 ± 98, | 303 ± 99, | 312 ± 104, | 273 ± 88, | 241 ± 58, | 233 ± 70, |
| | | n = 18 | n = 4 | n = 15 | n = 14 | n = 9 | n = 6 | n = 4 |
| | 2 mg/kg | 154 ± 63, | NA | 133 ± 38, | 139 ± 55, | 126 ± 51, | 142 ± 75, | 158 ± 110, |
| | | n = 18 | | n = 16 | n = 12 | n = 11 | n = 5 | n = 3 |
| | 4 mg/kg | 156 ± 66, | 176 ± 50, | 143 ± 66, | 132 ± 73, | 133 ± 69, | 130 ± 71, | 143 ± 73, |
| | | n = 17 | n = 7 | n = 15 | n = 11 | n = 12 | n = 8 | n = 6 |
| | 8 mg/kg | 119 ± 39, | 110 ± 26, | 107 ± 29, | 95 ± 27, | 92 ± 27, | 94 ± 32, | 91 ± 28, |
| | | n = 18 | n = 5 | n = 14 | n = 12 | n = 10 | n = 8 | n = 5 |

Data are presented as mean ± standard deviation (SD).
n = number of patients.
NA denotes samples where n < 3.

TABLE 19
Levels of IgA in Serum Compared to Baseline by Time and Dose following Administration of mAb 2419-1406

| | IgA (% Baseline) Mean ±SD, n | | | | | | | |
|---|---|---|---|---|---|---|---|---|
| | Month 0 | Month 0 | Month 0 | Month 0 | | Month 1 | | Month 2 |
| Arm | Day 1 | Day 3 | Day 8 | Day 18 | Month 1 | Day 18 | Month 2 | Day 18 |
| Placebo | 100.0 ± 0.0, | 99.4 ± 4.9, | 96.7 ± 6.7, | 100.9 ± 17.6, | 100.5 ± 14.3, | 91.7 ± 5.9, | 98.4 ± 8.8, | NA |
| | n = 27 | n = 7 | n = 26 | n = 25 | n = 26 | n = 4 | n = 22 | |
| 2 mg/kg | 100.0 ± 0.0, | 102.6 ± 15.6, | 89.0 ± 5.4, | 74.9 ± 7.7, | 66.6 ± 10.0, | 63.5 ± 5.9, | 57.1 ± 10.1, | 64.6 ± 8.6, |
| | n = 25 | n = 5 | n = 24 | n = 24 | n = 23 | n = 4 | n = 19 | n = 3 |
| 4 mg/kg | 100.0 ± 0.0, | 99.1 ± 6.4, | 91.5 ± 5.7, | 74.5 ± 8.9, | 61.3 ± 8.2, | 55.2 ± 7.6, | 47.1 ± 8.7, | 49.8 ± 7.3, |
| | n = 25 | n = 7 | n = 25 | n = 24 | n = 24 | n = 8 | n = 22 | n = 6 |
| 8 mg/kg | 100.0 ± 0.0, | 95.5 ± 6.1, | 88.9 ± 6.4, | 73.0 ± 8.5, | 59.2 ± 7.0, | 46.8 ± 4.0, | 44.3 ± 5.8, | 37.6 ± 4.6, |
| | n = 25 | n = 6 | n = 25 | n = 25 | n = 23 | n = 5 | n = 21 | n = 6 |
| | | IgA (% Baseline) Mean ±SD, n | | | | | | |
| | | | Month 3 | | | | | |
| | Arm | Month 3 | Day 18 | Month 4 | Month 5 | Month 6 | Month 7 | Month 8 |
| | Placebo | 99.9 ± 8.9, | 93.2 ± 7.2, | 94.6 ± 7.8, | 96.7 ± 8.2, | 94.7 ± 7.9, | 91.7 ± 4.7, | 91.2 ± 4.8, |
| | | n = 18 | n = 4 | n = 15 | n = 14 | n = 9 | n = 6 | n = 4 |
| | 2 mg/kg | 50.8 ± 11.3, | NA | 46.5 ± 8.2, | 47.4 ± 8.5, | 42.7 ± 7.5, | 40.9 ± 8.6, | 44.3 ± 13.0, |
| | | n = 18 | | n = 16 | n = 12 | n = 11 | n = 5 | n = 3 |
| | 4 mg/kg | 44.2 ± 7.2, | 47.3 ± 6.3, | 40.2 ± 8.3, | 38.0 ± 10.6, | 37.9 ± 11.6, | 35.2 ± 9.1, | 36.9 ± 5.0, |
| | | n = 17 | n = 7 | n = 15 | n = 11 | n = 12 | n = 8 | n = 6 |
| | 8 mg/kg | 38.2 ± 7.0, | 33.9 ± 5.3, | 35.0 ± 5.7, | 32.0 ± 6.2, | 30.5 ± 4.6, | 31.5 ± 6.8, | 26.6 ± 6.9, |
| | | n = 18 | n = 5 | n = 14 | n = 12 | n = 10 | n = 8 | n = 5 |

Data are presented as mean ± standard deviation (SD) percentage compared to baseline (month 0 day 1; prior to administration of mAb 2419-1406), n = number of patients.
NA denotes samples where n < 3.

TABLE 20
Number (%) of Patients with ≥60% IgA Reduction from Baseline by Dose and Time
following Administration of mAb 2419-1406

| | Number (%) of subjects with ≥60% IgA reduction from baseline | | | | | | |
|---|---|---|---|---|---|---|---|
| | Month 0 | Month 0 | Month 0 | | Month 1 | | Month 2 |
| Arm | Day 3 | Day 8 | Day 18 | Month 1 | Day 18 | Month 2 | Day 18 |
| Placebo | 0 out of 7 | 0 out of 26 | 0 out of 25 | 0 out of 26 | 0 out of 4 | 0 out of 22 | NA |
| | (0.0%) | (0.0%) | (0.0%) | (0.0%) | (0.0%) | (0.0%) | |
| 2 mg/kg | 0 out of 5 | 0 out of 24 | 0 out of 24 | 0 out of 23 | 0 out of 4 | 1 out of 19 | 0 out of 3 |
| | (0.0%) | (0.0%) | (0.0%) | (0.0%) | (0.0%) | (5.3%) | (0.0%) |
| 4 mg/kg | 0 out of 7 | 0 out of 25 | 0 out of 24 | 0 out of 24 | 0 out of 8 | 5 out of 22 | 0 out of 6 |
| | (0.0%) | (0.0%) | (0.0%) | (0.0%) | (0.0%) | (22.7%) | (0.0%) |
| 8 mg/kg | 0 out of 6 | 0 out of 25 | 0 out of 25 | 0 out of 23 | 0 out of 5 | 5 out of 21 | 4 out of 6 |
| | (0.0%) | (0.0%) | (0.0%) | (0.0%) | (0.0%) | (23.8%) | (66.7%) |
| | Number (%) of subjects with ≥60% IgA reduction from baseline | | | | | | |
| | | Month 3 | | | | | |
| Arm | Month 3 | Day 18 | Month 4 | Month 5 | Month 6 | Month 7 | Month 8 |
| Placebo | 0 out of 18 | 0 out of 4 | 0 out of 15 | 0 out of 14 | 0 out of 9 | 0 out of 6 | 0 out of 4 |
| | (0.0%) | (0.0%) | (0.0%) | (0.0%) | (0.0%) | (0.0%) | (0.0%) |
| 2 mg/kg | 3 out of 18 | NA | 3 out of 16 | 3 out of 12 | 4 out of 11 | 2 out of 5 | 1 out of 3 |
| | (16.7%) | | (18.8%) | (25.0%) | (36.4%) | (40.0%) | (33.3%) |
| 4 mg/kg | 2 out of 17 | 1 out of 7 | 7 out of 15 | 7 out of 11 | 8 out of 12 | 6 out of 8 | 5 out of 6 |
| | (11.8%) | (14.3%) | (46.7%) | (63.6%) | (66.7%) | (75.0%) | (83.3%) |
| 8 mg/kg | 11 out of 18 | 4 out of 5 | 11 out of 14 | 10 out of 12 | 10 out of 10 | 7 out of 8 | 5 out of 5 |
| | (61.1%) | (80.0%) | (78.6%) | (83.3%) | (100.0%) | (87.5%) | (100.0%) |

NA denotes samples where number of patients n < 3.

TABLE 21
Number (%) of Patients with ≥40% IgA Reduction from Baseline by Dose and Time
following Administration of mAb 2419-1406

| | Number (%) of subjects with ≥40% IgA reduction from baseline | | | | | | |
|---|---|---|---|---|---|---|---|
| | Month 0 | Month 0 | Month 0 | | Month 1 | | Month 2 |
| Arm | Day 3 | Day 8 | Day 18 | Month 1 | Day 18 | Month 2 | Day 18 |
| Placebo | 0 out of 7 | 0 out of 26 | 0 out of 25 | 0 out of 2 | 0 out of 4 | 0 out of 22 | NA |
| | (0.0%) | (0.0%) | (0.0%) | (0.0%) | (0.0%) | (0.0%) | |
| 2 mg/kg | 0 out of 5 | 0 out of 24 | 0 out of 24 | 5 out of 23 | 1 out of 4 | 13 out of 19 | 1 out of 3 |
| | (0.0%) | (0.0%) | (0.0%) | (21.7%) | (25.0%) | (68.4%) | (33.3%) |
| 4 mg/kg | 0 out of 7 | 0 out of 25 | 1 out of 24 | 9 out of 24 | 7 out of 8 | 20 out of 22 | 5 out of 6 |
| | (0.0%) | (0.0%) | (4.2%) | (37.5%) | (87.5%) | (90.9%) | (83.3%) |
| 8 mg/kg | 0 out of 6 | 0 out of 25 | 0 out of 25 | 16 out of 23 | 5 out of 5 | 21 out of 21 | 6 out of 6 |
| | (0.0%) | (0.0%) | (0.0%) | (69.6%) | (100.0%) | (100.0%) | (100.0%) |
| | Number (%) of subjects with ≥40% IgA reduction from baseline | | | | | | |
| | | Month 3 | | | | | |
| Arm | Month 3 | Day 18 | Month 4 | Month 5 | Month 6 | Month 7 | Month 8 |
| Placebo | 0 out of 18 | 0 out of 4 | 0 out of 15 | 0 out of 14 | 0 out of 9 | 0 out of 6 | 0 out of 4 |
| | (0.0%) | (0.0%) | (0.0%) | (0.0%) | (0.0%) | (0.0%) | (0.0%) |
| 2 mg/kg | 16 out of 18 | NA | 16 out of 16 | 11 out of 12 | 11 out of 11 | 5 out of 5 | 3 out of 3 |
| | (88.9%) | | (100.0%) | (91.7%) | (100.0%) | (100.0%) | (100.0%) |
| 4 mg/kg | 17 out of 17 | 7 out of 7 | 15 out of 15 | 11 out of 11 | 12 out of 12 | 8 out of 8 | 6 out of 6 |
| | (100.0%) | (100.0%) | (100.0%) | (100.0%) | (100.0%) | 100.0%) | (100.0%) |
| 8 mg/kg | 18 out of 18 | 5 out of 5 | 14 out of 14 | 12 out of 12 | 10 out of 10 | 8 out of 8 | 5 out of 5 |
| | (100.0%) | (100.0%) | (100.0%) | (100.0%) | (100.0%) | (100.0%) | (100.0%) |

NA denotes samples where number of patients n < 3.

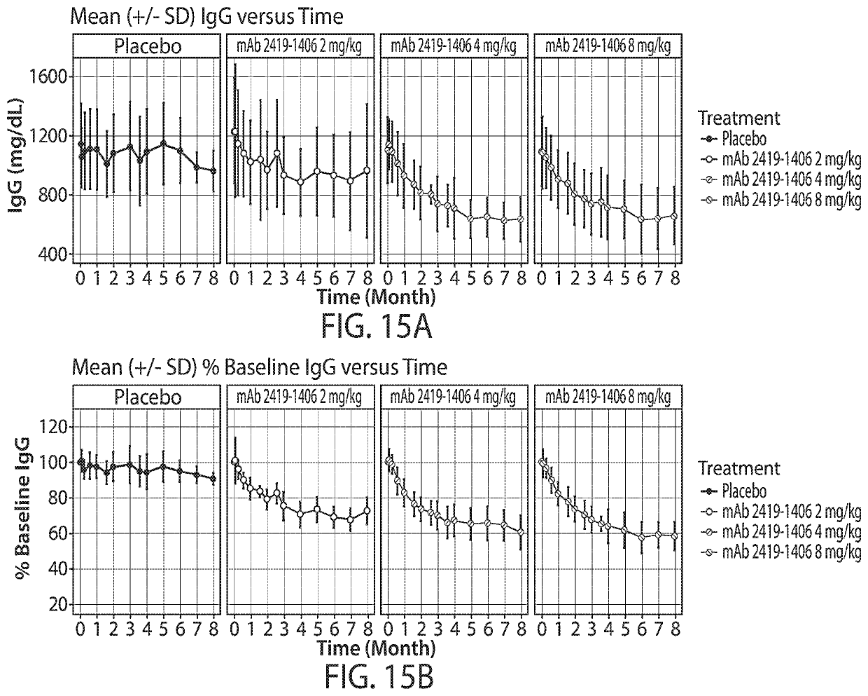

Administration of mAb 2419-1406 also led to suppression of IgG levels. As shown in FIG. 15A and Table 22, levels of serum IgG decreased over time in a dose-dependent manner following mAb 2419-1406 administration. When levels of serum IgG were compared to baseline levels, patients administered with placebo exhibited minimal changes in serum IgG levels, with the largest reduction (approximately 10% reduction from baseline level) observed at the month 8 time point (FIG. 15B and Table 23). In contrast, dose-dependent reductions in IgG levels were observed in patients administered with mAb 2419-1406. By the month 1 time point, mean serum IgG levels were 85.3% for the 2 mg/kg group, 82.9% for the 4 mg/kg group, and 82.3% for the 8 mg/kg group compared to baseline (FIG. 15B and Table 23).

Reductions in serum IgG levels continued over time, and by the month 8 time point, mean IgG levels were 72.7% for the 2 mg/kg group, 60.8% for the 4 mg/kg group, and 58.4% for the 8 mg/kg group compared to baseline (FIG. 15B and Table 23). Across all time points assayed, patients in the 8 mg/kg dose group exhibited the greatest reductions in serum IgG levels compared to the other dose groups, and patients in the 4 mg/kg dose group exhibited greater reductions in serum IgG levels as compared to the 2 mg/kg dose group (FIG. 15B, Table 23). The reductions in IgG levels are of a lower magnitude than drops in IgA levels, with at most approximately 40% reduction compared to baseline; however, the reductions in IgG levels upon mAb 2419-1406 administration are greater than those observed in the placebo group (FIGS. 14B and 15B).

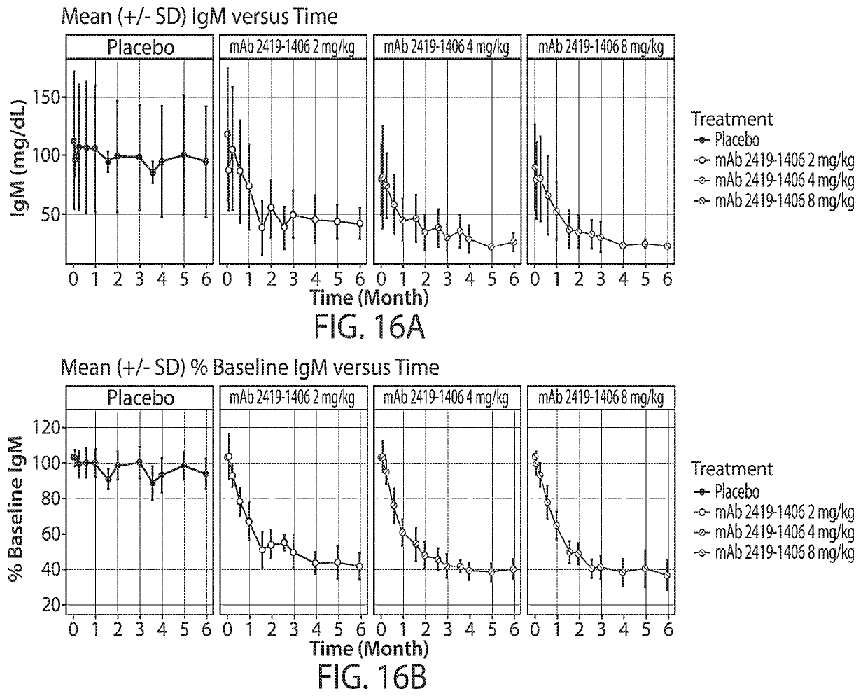

IgM levels were reduced following administration of mAb 2419-1406. As shown in FIG. 16A and Table 24, levels of serum IgM decreased over time in a dose-dependent manner following mAb 2419-1406 administration. When serum IgM levels were compared to baseline levels, patients administered with placebo exhibited minimal changes in serum IgM levels (FIG. 16B and Table 25). In contrast, dose-dependent reductions in IgM levels were observed in patients administered with mAb 2419-1406 (FIG. 16B and Table 25). By the month 1 time point, mean serum IgM levels were 60.6% for the 2 mg/kg group, 53.9% for the 4 mg/kg group, and 58.1% for the 8 mg/kg group compared to baseline (FIG. 16B and Table 25). Reductions in serum IgM levels continued overtime, and by the month 8 time point, mean IgM levels were 32.8% for the 2 mg/kg group, 31.2% for the 4 mg/kg group, and 27.5% for the 8 mg/kg group compared to baseline (FIG. 16B and Table 25). Reductions in serum IgM levels were comparable between the 8 mg/kg and 4 mg/kg mAb 2419-1406 dose groups, and the reductions observed in the 8 mg/kg and 4 mg/kg dose groups were greater than those observed in the 2 mg/kg dose group (FIG. 16B and Table 25). The reductions in IgM are close to, or slightly greater, than the reductions in IgA (FIGS. 14B and 16B).

In summary, administration of mAb 2419-1406 to human IgA neuropathy patients led to dose-dependent reductions of serum IgA, IgG, and IgM levels overtime.

TABLE 22
Concentration of IgG in Serum by Time and Dose following Administration of mAb 2419-1406

| | IgG (mg/dL) Mean ± SD, n | | | | | | | |
|---|---|---|---|---|---|---|---|---|
| | Month 0 | Month 0 | Month 0 | Month 0 | | Month 1 | | Month 2 |
| Arm | Day 1 | Day 3 | Day 8 | Day 18 | Month 1 | Day 18 | Month 2 | Day 18 |
| Placebo | 1144 ± 273, | 1051 ± 204, | 1095 ± 262, | 1110 ± 269, | 1107 ± 271, | 1010 ± 222, | 1082 ± 262, | NA |
| | n = 27 | n = 7 | n = 26 | n = 25 | n = 26 | n = 4 | n = 22 | |
| 2 mg/kg | 1232 ± 356, | 1227 ± 445, | 1149 ± 304, | 1078 ± 294, | 1019 ± 278, | 1034 ± 406, | 966 ± 263, | 1078 ± 363, |
| | n = 25 | n = 5 | n = 24 | n = 24 | n = 23 | n = 4 | n = 19 | n = 3 |
| 4 mg/kg | 1100 ± 226, | 1140 ± 172, | 1090 ± 207, | 1009 ± 212, | 930 ± 216, | 868 ± 166, | 811 ± 182, | 802 ± 64, |
| | n = 25 | n = 7 | n = 25 | n = 24 | n = 24 | n = 8 | n = 22 | n = 6 |
| 8 mg/kg | 1091 ± 229, | 1081 ± 243, | 1049 ± 208, | 984 ± 219, | 905 ± 196, | 874 ± 207, | 805 ± 201, | 773 ± 194, |
| | n = 25 | n = 6 | n = 25 | n = 25 | n = 23 | n = 5 | n = 21 | n = 6 |
| | | IgG (mg/dL) Mean ± SD, n | | | | | | |
| | | | Month 3 | | | | | |
| | Arm | Month 3 | Day 18 | Month 4 | Month 5 | Month 6 | Month 7 | Month 8 |
| | Placebo | 1127 ± 299, | 1028 ± 302, | 1092 ± 293, | 1146 ± 277, | 1099 ± 221, | 981 ± 105, | 960 ± 140, |
| | | n = 18 | n = 4 | n = 15 | n = 14 | n = 9 | n = 6 | n = 4 |
| | 2 mg/kg | 931 ± 264, | NA | 885 ± 229, | 957 ± 302, | 930 ± 279, | 893 ± 333, | 962 ± 450, |
| | | n = 18 | | n = 16 | n = 12 | n = 10 | n = 5 | n = 3 |
| | 4 mg/kg | 740 ± 185, | 725 ± 141, | 709 ± 205, | 638 ± 128, | 650 ± 133, | 627 ± 123, | 636 ± 148, |
| | | n = 17 | n = 7 | n = 15 | n = 11 | n = 12 | n = 8 | n = 6 |
| | 8 mg/kg | 740 ± 202, | 749 ± 230, | 713 ± 214, | 702 ± 195, | 634 ± 234, | 640 ± 201, | 659 ± 195, |
| | | n = 18 | n = 5 | n = 14 | n = 12 | n = 10 | n = 8 | n = 5 |

Data are presented as mean ± standard deviation (SD).
n = number of patients.
NA denotes samples where size n < 3.

TABLE 23
Levels of IgG in Serum Compared to Baseline by Time and Dose following Administration of mAb 2419-1406

| | IgG (% Baseline) Mean ± SD, n | | | | | | | |
|---|---|---|---|---|---|---|---|---|
| | Month 0 | Month 0 | Month 0 | Month 0 | | Month 1 | | Month 2 |
| Arm | Day 1 | Day 3 | Day 8 | Day 18 | Month 1 | Day 18 | Month 2 | Day 18 |
| Placebo | 100.0 ± 0.0, | 100.9 ± 6.1, | 95.9 ± 5.5, | 97.8 ± 7.7, | 97.4 ± 6.3, | 94.1 ± 6.7, | 97.4 ± 7.8, | NA |
| | n = 27 | n = 7 | n = 26 | n = 25 | n = 26 | n = 4 | n = 22 | |
| 2 mg/kg | 100.0 ± 0.0, | 101.0 ± 12.6, | 96.2 ± 5.7, | 90.1 ± 4.7, | 85.3 ± 6.6, | 83.6 ± 3.1, | 79.3 ± 5.6, | 82.8 ± 5.6, |
| | n = 25 | n = 5 | n = 24 | n = 24 | n = 23 | n = 4 | n = 19 | n = 3 |
| 4 mg/kg | 100.0 ± 0.0, | 101.4 ± 6.5, | 98.6 ± 5.5, | 89.7 ± 7.5, | 82.9 ± 7.3, | 76.6 ± 6.7, | 73.8 ± 6.9, | 71.6 ± 7.0, |
| | n = 25 | n = 7 | n = 25 | n = 24 | n = 24 | n = 8 | n = 22 | n = 6 |
| 8 mg/kg | 100.0 ± 0.0, | 99.3 ± 8.3, | 96.4 ± 5.7, | 90.0 ± 7.2, | 82.3 ± 6.4, | 77.8 ± 8.1, | 73.8 ± 7.1, | 70.6 ± 7.8, |
| | n = 25 | n = 6 | n = 25 | n = 25 | n = 23 | n = 5 | n = 21 | n = 6 |
| | | IgG (% Baseline) Mean ± SD, n | | | | | | |
| | | | Month 3 | | | | | |
| | Arm | Month 3 | Day 18 | Month 4 | Month 5 | Month 6 | Month 7 | Month 8 |
| | Placebo | 98.6 ± 10.4, | 94.8 ± 8.5, | 94.3 ± 9.9, | 97.5 ± 8.3, | 95.0 ± 6.2, | 93.0 ± 4.3, | 90.8 ± 3.3, |
| | | n = 18 | n = 4 | n = 15 | n = 14 | n = 9 | n = 6 | n = 4 |
| | 2 mg/kg | 75.7 ± 7.9, | NA | 71.0 ± 6.9, | 73.5 ± 7.4, | 69.2 ± 5.7, | 67.8 ± 6.3, | 72.7 ± 7.7, |
| | | n = 18 | | n = 16 | n = 12 | n = 10 | n = 5 | n = 3 |
| | 4 mg/kg | 70.2 ± 7.8, | 66.2 ± 8.6, | 67.3 ± 8.8, | 65.4 ± 9.1, | 65.8 ± 9.5, | 64.8 ± 8.3, | 60.8 ± 9.4, |
| | | n = 17 | n = 7 | n = 15 | n = 11 | n = 12 | n = 8 | n = 6 |
| | 8 mg/kg | 67.8 ± 6.9, | 65.3 ± 4.0, | 64.2 ± 9.4, | 62.0 ± 9.9, | 57.9 ± 8.6, | 59.4 ± 7.2, | 58.4 ± 8.3, |
| | | n = 18 | n = 5 | n = 14 | n = 12 | n = 10 | n = 8 | n = 5 |

Data are presented as mean ± standard deviation (SD) percentage compared to baseline (month 0 day 1; prior to administration of mAb 2419-1406), n = number of patients.
NA denotes samples where n < 3.

TABLE 24
Concentration of IgM in Serum by Time and Dose following Administration of mAb 2419-1406

| | IgM (mg/dL) Mean ± SD, n | | | | | | |
|---|---|---|---|---|---|---|---|
| | Month 0 | Month 0 | Month 0 | Month 0 | | Month 1 | |
| Arm | Day 1 | Day 3 | Day 8 | Day 18 | Month 1 | Day 18 | Month 2 |
| Placebo | 113 ± 59, | 97 ± 15, | 108 ± 54, | 107 ± 57, | 106 ± 54, | 96 ± 9, | 100 ± 48, |
| | n = 27 | n = 7 | n = 26 | n = 25 | n = 26 | n = 4 | n = 22 |
| 2 mg/kg | 118 ± 57, | 87 ± 34, | 106 ± 53, | 87 ± 44, | 74 ± 36, | 39 ± 23, | 56 ± 25, |
| | n = 25 | n = 5 | n = 24 | n = 24 | n = 23 | n = 4 | n = 19 |
| 4 mg/kg | 80 ± 30, | 82 ± 44, | 74 ± 27, | 59 ± 25, | 45 ± 18, | 47 ± 20, | 35 ± 15, |
| | n = 25 | n = 7 | n = 25 | n = 24 | n = 23 | n = 6 | n = 18 |
| 8 mg/kg | 90 ± 37, | 79 ± 34, | 81 ± 36, | 67 ± 33, | 53 ± 25, | 37 ± 16, | 35 ± 14, |
| | n = 25 | n = 6 | n = 25 | n = 25 | n = 23 | n = 5 | n = 19 |
| | | IgM (mg/dL) Mean ±S D, n | | | | | |
| | | Month 2 | | Month 3 | | | |
| | Arm | Day 18 | Month 3 | Day 18 | Month 4 | Month 5 | Month 6 |
| | Placebo | NA | 99 ± 46, | 86 ± 9, | 95 ± 48, | 101 ± 51, | 95 ± 47, |
| | | | n = 18 | n = 4 | n = 15 | n = 14 | n = 9 |
| | 2 mg/kg | 39 ± 18, | 50 ± 20, | NA | 45 ± 20, | 44 ± 14, | 42 ± 13, |
| | | n = 3 | n = 18 | | n = 15 | n = 11 | n = 9 |
| | 4 mg/kg | 39 ± 16, | 30 ± 11, | 36 ± 13, | 29 ± 12, | 22 ± 2, | 27 ± 8, |
| | | n = 5 | n = 13 | n = 5 | n = 9 | n = 6 | n = 7 |
| | 8 mg/kg | 33 ± 12, | 31 ± 13, | NA | 24 ± 2, | 25 ± 4, | 23 ± 1, |
| | | n = 3 | n = 12 | | n = 8 | n = 6 | n = 4 |

Data are presented as mean ± standard deviation (SD).
n = number of patients.
NA denotes samples where n < 3.

TABLE 25
Levels of IgM in Serum Compared to Baseline by Time and Dose following Administration of mAb 2419-1406

| | IgM (% Baseline) Mean ± SD, n | | | | | | |
|---|---|---|---|---|---|---|---|
| | Month 0 | Month 0 | Month 0 | Month 0 | | Month 1 | |
| Arm | Day 1 | Day 3 | Day 8 | Day 18 | Month 1 | Day 18 | Month 2 |
| Placebo | 100.0 ± 0.0, | 99.7 ± 5.0, | 95.9 ± 8.3, | 96.8 ± 9.0, | 96.5 ± 8.6, | 86.4 ± 6.3, | 94.9 ± 8.8, |
| | n = 27 | n = 7 | n = 26 | n = 25 | n = 26 | n = 4 | n = 22 |
| 2 mg/kg | 100.0 ± 0.0, | 100.9 ± 14.0, | 88.9 ± 6.9, | 72.8 ± 8.5, | 60.6 ± 11.5, | 43.1 ± 10.9, | 46.2 ± 8.8, |
| | n = 25 | n = 5 | n = 24 | n = 24 | n = 23 | n = 4 | n = 19 |
| 4 mg/kg | 100.0 ± 0.0, | 100.0 ± 9.4, | 91.1 ± 7.2, | 70.3 ± 11.1, | 53.9 ± 8.3, | 46.5 ± 10.4, | 39.5 ± 8.1, |
| | n = 25 | n = 7 | n = 25 | n = 24 | n = 23 | n = 6 | n = 18 |
| 8 mg/kg | 100.0 ± 0.0, | 96.3 ± 7.3, | 89.1 ± 7.4, | 72.5 ± 10.2, | 58.1 ± 8.6, | 41.8 ± 6.7, | 40.6 ± 6.8, |
| | n = 25 | n = 6 | n = 25 | n = 25 | n = 23 | n = 5 | n = 19 |
| | | IgM (% Baseline) Mean ± SD, n | | | | | |
| | | Month 2 | | Month 3 | | | |
| | Arm | Day 18 | Month 3 | Day 18 | Month 4 | Month 5 | Month 6 |
| | Placebo | NA | 96.8 ± 9.7, | 84.2 ± 10.8, | 89.2 ± 10.8, | 94.8 ± 8.6, | 89.9 ± 9.7, |
| | | | n = 18 | n = 4 | n = 15 | n = 14 | n = 9 |
| | 2 mg/kg | 47.4 ± 4.6, | 41.8 ± 10.0, | NA | 34.9 ± 6.9, | 35.3 ± 10.0, | 32.8 ± 8.3, |
| | | n = 3 | n = 18 | | n = 15 | n = 11 | n = 9 |
| | 4 mg/kg | 37.3 ± 6.7, | 33.1 ± 7.6, | 32.9 ± 3.9, | 30.1 ± 5.6, | 29.3 ± 5.6, | 31.2 ± 6.1, |
| | | n = 5 | n = 13 | n = 5 | n = 9 | n = 6 | n = 7 |
| | 8 mg/kg | 31.6 ± 5.4, | 32.2 ± 6.9, | NA | 29.2 ± 8.4, | 31.6 ± 11.4, | 27.5 ± 9.4, |
| | | n = 3 | n = 12 | | n = 8 | n = 6 | n = 4 |

Data are presented as mean ± standard deviation (SD) percentage compared to baseline (month 0 day 1; prior to administration of mAb 2419-1406), n = number of patients.
NA denotes samples where n < 3.

### Example 8. Effects of mAb 2419-1406 on Proteinuria

This Example describes that administration of mAb 2419-1406 to IgA neuropathy patients resulted in a reduction of protein excretion in urine.

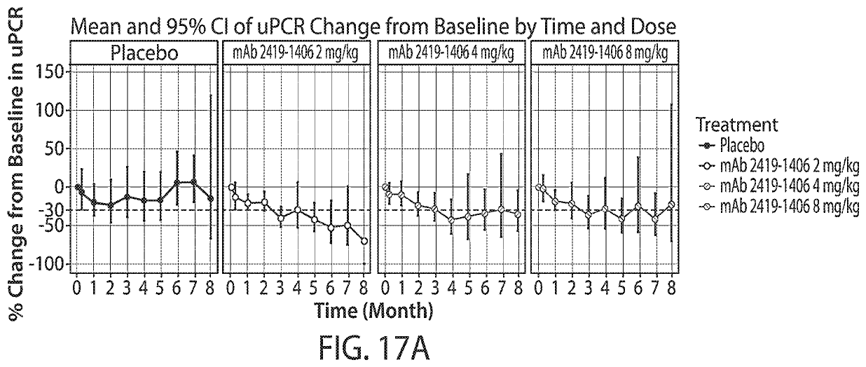

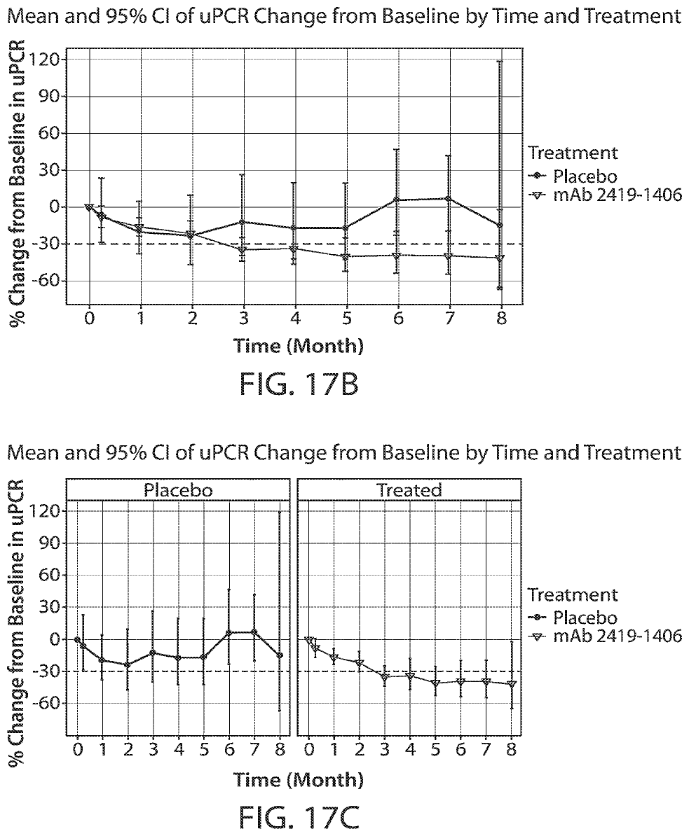

In this example, IgA nephropathy patients were randomized to receive a weight-adjusted dose of placebo, 2 mg/kg, 4 mg/kg, or 8 mg/kg of mAb 2419-1406 once a month. At the time points indicated in FIG. 17, spot urine protein:creatinine ratios (uPCR) were measured. Percent change from baseline (PCFB) within each dose group was calculated using the following formula: PCFB=100*[exp(mean(log(t₁/t₀)))-1], where to is the uPCR at baseline (t=0) and tᵢ is the uPCR at time points after t=0. The PCFB at baseline (t=0) is set to 0% within every dose group. Mean and 95% confidence intervals of PCFB were calculated. Later time point data were masked to avoid the risk of unblinding.

As shown in FIG. 17A, the PCFB in uPCR of patients on placebo stayed close to 0 over the course of the study. On the other hand, patients in the 2 mg/kg, 4 mg/kg, and 8 mg/kg mAb 2419-1406 dose groups showed approximately 30% or greater decrease in uPCR when the dose groups were examined individually (FIG. 17A) or pooled together (FIGS. 17B and 17C). While only 25.0% of the patients at placebo exhibited>40% reduction in uPCR from baseline at the month 8 time point, 33.3%, 33.3%, and 40.0% of the patients in the 2 mg/kg, 4 mg/kg, and 8 mg/kg dose groups, respectively, exhibited>40% uPCR reduction at the same time point (Table 26). When the threshold was set to >30% uPCR reduction from baseline, 33.3%, 50.0%, and 40.0% of the patients in the 2 mg/kg, 4 mg/kg, and 8 mg/kg dose groups, respectively, achieved the threshold at the month 8 time point, while only 25.0% of the patients with placebo achieved the threshold (Table 27). When the threshold was set to >20% uPCR reduction from baseline, 100%, 66.7%, and 40.0% of the patients in the 2 mg/kg, 4 mg/kg, and 8 mg/kg dose groups, respectively, achieved the threshold at the month 8 time point, while only 25.0% of the patients with placebo achieved the threshold (Table 28). When the threshold was set to >10% uPCR reduction from baseline, 100%, 83.3%, and 40.0% of the patients in the 2 mg/kg, 4 mg/kg, and 8 mg/kg dose groups, respectively, achieved the threshold at the month 8 time point, while only 25.0% of the patients with placebo achieved the threshold (Table 29).

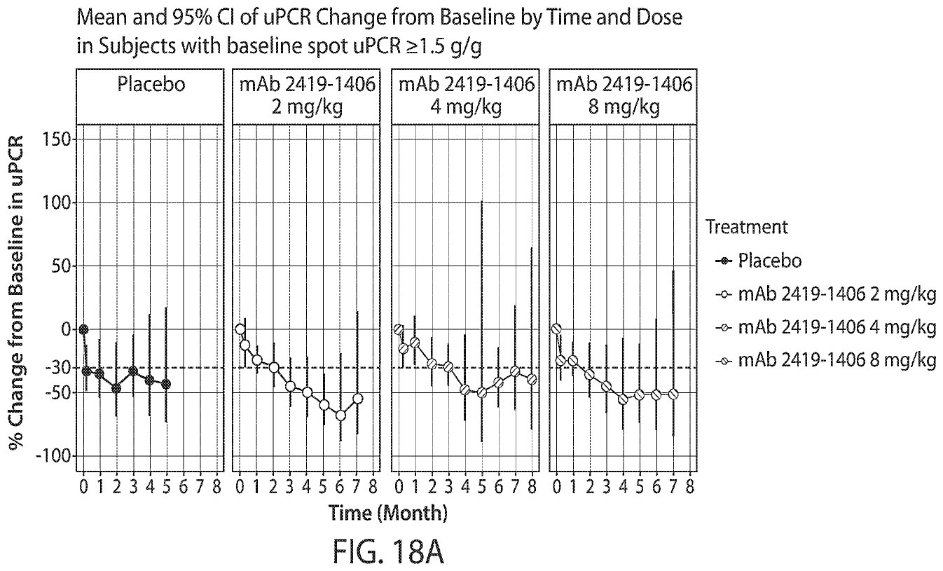

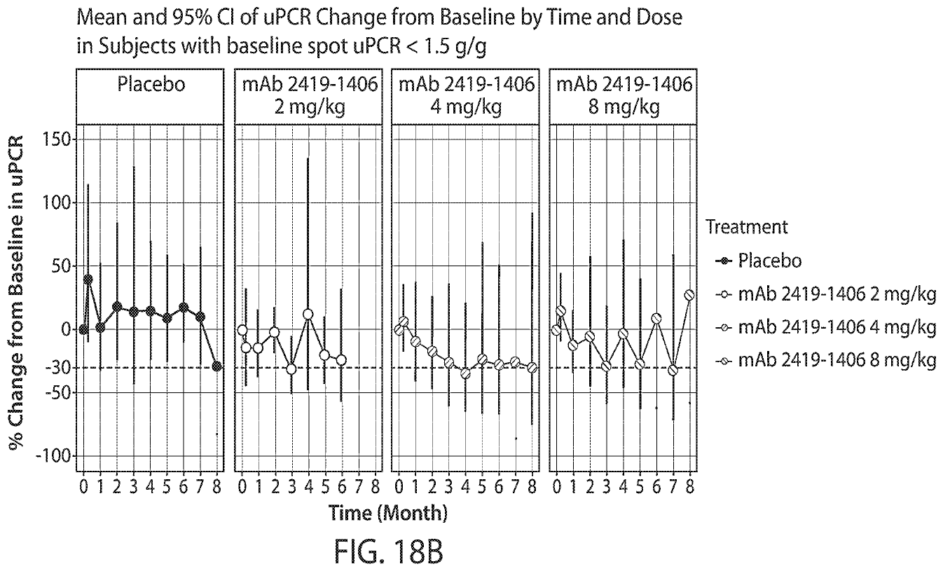

Preliminary subgroup analyses were conducted based upon patient proteinuria levels prior to administration of mAb 2419-1406. Patients with heavy proteinuria (defined as >1.5 g/g; FIG. 18A) or less proteinuria (defined as <1.5 g/g; FIG. 18B) prior to receiving mAb 2419-1406 administration were observed to benefit from receiving mAb 2419-1406 as assessed by spot uPCR.

In summary, administration of mAb 2419-1406 to human IgA neuropathy patients led to decreased protein excretion in urine and reduction in proteinuria as assessed by spot uPCR.

TABLE 26
Number (%) of Subjects with ≥40% uPCR Reduction from Baseline by Dose and Time

| | Number (%) of subjects with ≥40% uPCR reduction from baseline | | | | | | | | | |
|---|---|---|---|---|---|---|---|---|---|---|
| | Month 0 | Month 0 | | | | | | | | |
| Arm | Day 1 | Day 8 | Month 1 | Month 2 | Month 3 | Month 4 | Month 5 | Month 6 | Month 7 | Month 8 |
| Placebo | 0 out of 25 | 5 out of 22 | 5 out of 23 | 6 out of 20 | 4 out of 16 | 3 out of 14 | 2 out of 12 | 1 out of 8 | 0 out of 5 | 1 out of 4 |
| | (0.0%) | (22.7%) | (21.7%) | (30.0%) | (25.0%) | (21.4%) | (16.7%) | (12.5%) | (0.0%) | (25.0%) |
| 2 mg/kg | 0 out of 25 | 3 out of 22 | 4 out of 22 | 2 out of 19 | 8 out of 18 | 6 out of 14 | 6 out of 13 | 5 out of 11 | 2 out of 5 | 1 out of 3 |
| | (0.0%) | (13.6%) | (18.2%) | (10.5%) | (44.4%) | (42.9%) | (46.2%) | (45.5%) | (40.0%) | (33.3%) |
| 4 mg/kg | 0 out of 25 | 2 out of 24 | 5 out of 23 | 7 out of 21 | 5 out of 16 | 8 out of 14 | 4 out of 10 | 4 out of 10 | 2 out of 7 | 2 out of 6 |
| | (0.0%) | (8.3%) | (21.7%) | (33.3%) | (31.2%) | (57.1%) | (40.0%) | (40.0%) | (28.6%) | (33.3%) |
| 8 mg/kg | 0 out of 24 | 2 out of 23 | 4 out of 22 | 4 out of 19 | 8 out of 17 | 4 out of 13 | 5 out of 10 | 3 out of 9 | 4 out of 7 | 2 out of 5 |
| | (0.0%) | (8.7%) | (18.2%) | (21.1%) | (47.1%) | (30.8%) | (50.0%) | (33.3%) | (57.1%) | (40.0%) |

Data are presented as the number (and percentage) of patients that achieved ≥40% spot urine protein:creatinine ratios (uPCR) reduction from baseline out of total observations.

TABLE 27
Number (%) of Subjects with ≥30% uPCR Reduction from Baseline by Dose and Time

| | Number (%) of subjects with ≥30% uPCR reduction from baseline | | | | | | | | | |
|---|---|---|---|---|---|---|---|---|---|---|
| | Month 0 | Month 0 | | | | | | | | |
| Arm | Day 1 | Day 8 | Month 1 | Month 2 | Month 3 | Month 4 | Month 5 | Month 6 | Month 7 | Month 8 |
| Placebo | 0 out of 25 | 8 out of 22 | 7 out of 23 | 9 out of 20 | 7 out of 16 | 4 out of 14 | 4 out of 12 | 1 out of 8 | 0 out of 5 | 1 out of 4 |
| | (0.0%) | (36.4%) | (30.4%) | (45.0%) | (43.8%) | (28.6%) | (33.3%) | (12.5%) | (0.0%) | (25.0%) |
| 2 mg/kg | 0 out of 25 | 6 out of 22 | 6 out of 22 | 6 out of 19 | 9 out of 18 | 7 out of 14 | 8 out of 13 | 6 out of 11 | 3 out of 5 | 1 out of 3 |
| | (0.0%) | (27.3%) | (27.3%) | (31.6%) | (50.0%) | (50.0%) | (61.5%) | (54.5%) | (60.0%) | (33.3%) |
| 4 mg/kg | 0 out of 25 | 6 out of 24 | 8 out of 23 | 9 out of 21 | 5 out of 16 | 8 out of 14 | 4 out of 10 | 5 out of 10 | 2 out of 7 | 3 out of 6 |
| | (0.0%) | (25.0%) | (34.8%) | (42.9%) | (31.2%) | (57.1%) | (40.0%) | (50.0%) | (28.6%) | (50.0%) |
| 8 mg/kg | 0 out of 24 | 5 out of 23 | 6 out of 22 | 10 out of 19 | 9 out of 17 | 6 out of 13 | 7 out of 10 | 6 out of 9 | 5 out of 7 | 2 out of 5 |
| | (0.0%) | (21.7%) | (27.3%) | (52.6%) | (52.9%) | (46.2%) | (70.0%) | (66.7%) | (71.4%) | (40.0%) |

Data are presented as the number (and percentage) of patients that achieved ≥30% spot urine protein:creatinine ratios (uPCR) reduction from baseline out of total observations.

TABLE 28
Number (%) of Subjects with ≥20% uPCR Reduction from Baseline by Dose and Time

| | Number (%) of subjects with ≥20% uPCR reduction from baseline | | | | | | | | | |
|---|---|---|---|---|---|---|---|---|---|---|
| | Month 0 | Month 0 | | | | | | | | |
| Arm | Day 1 | Day 8 | Month 1 | Month 2 | Month 3 | Month 4 | Month 5 | Month 6 | Month 7 | Month 8 |
| Placebo | 0 out of 25 | 9 out of 22 | 11 out of 23 | 11 out of 20 | 8 out of 16 | 5 out of 14 | 5 out of 12 | 1 out of 8 | 1 out of 5 | 1 out of 4 |
| | (0.0%) | (40.9%) | (47.8%) | (55.0%) | (50.0%) | (35.7%) | (41.7%) | (12.5%) | (20.0%) | (25.0%) |
| 2 mg/kg | 0 out of 25 | 6 out of 22 | 12 out of 22 | 8 out of 19 | 11 out of 18 | 7 out of 14 | 9 out of 13 | 6 out of 11 | 4 out of 5 | 3 out of 3 |
| | (0.0%) | (27.3%) | (54.5%) | (42.1%) | (61.1%) | (50.0%) | (69.2%) | (54.5%) | (80.0%) | (100.0%) |
| 4 mg/kg | 0 out of 25 | 8 out of 24 | 9 out of 23 | 10 out of 21 | 10 out of 16 | 9 out of 14 | 5 out of 10 | 8 out of 10 | 4 out of 7 | 4 out of 6 |
| | (0.0%) | (33.3%) | (39.1%) | (47.6%) | (62.5%) | (64.3%) | (50.0%) | (80.0%) | (57.1%) | (66.7%) |
| 8 mg/kg | 0 out of 24 | 6 out of 23 | 10 out of 22 | 12 out of 19 | 13 out of 17 | 9 out of 13 | 8 out of 10 | 6 out of 9 | 6 out of 7 | 2 out of 5 |
| | (0.0%) | (26.1%) | (45.5%) | (63.2%) | (76.5%) | (69.2%) | (80.0%) | (66.7%) | (85.7%) | (40.0%) |

Data are presented as the number (and percentage) of patients that achieved ≥20% spot urine protein:creatinine ratios (uPCR) reduction from baseline out of total observations.

TABLE 29
Number (%) of Subjects with ≥10% uPCR Reduction from Baseline by Dose and Time

| | Number (%) of subjects with ≥10% uPCR reduction from baseline | | | | | | | | | |
|---|---|---|---|---|---|---|---|---|---|---|
| | Month 0 | Month 0 | | | | | | | | |
| Arm | Day 1 | Day 8 | Month 1 | Month 2 | Month 3 | Month 4 | Month 5 | Month 6 | Month 7 | Month 8 |
| Placebo | 0 out of 25 | 11 out of 22 | 11 out of 23 | 11 out of 20 | 9 out of 16 | 5 out of 14 | 6 out of 12 | 1 out of 8 | 1 out of 5 | 1 out of 4 |
| | (0.0%) | (50.0%) | (47.8%) | (55.0%) | (56.2%) | (35.7%) | (50.0%) | (12.5%) | (20.0%) | (25.0%) |
| 2 mg/kg | 0 out of 25 | 10 out of 22 | 17 out of 22 | 11 out of 19 | 16 out of 18 | 7 out of 14 | 11 out of 13 | 9 out of 11 | 5 out of 5 | 3 out of 3 |
| | (0.0%) | (45.5%) | (77.3%) | (57.9%) | (88.9%) | (50.0%) | (84.6%) | (81.8%) | (100.0%) | (100.0%) |
| 4 mg/kg | 0 out of 25 | 12 out of 24 | 10 out of 23 | 11 out of 21 | 12 out of 16 | 9 out of 14 | 5 out of 10 | 9 out of 10 | 5 out of 7 | 5 out of 6 |
| | (0.0%) | (50.0%) | (43.5%) | (52.4%) | (75.0%) | (64.3%) | (50.0%) | (90.0%) | (71.4%) | (83.3%) |
| 8 mg/kg | 0 out of 24 | 10 out of 23 | 15 out of 22 | 12 out of 19 | 13 out of 17 | 11 out of 13 | 9 out of 10 | 6 out of 9 | 6 out of 7 | 2 out of 5 |
| | (0.0%) | (43.5%) | (68.2%) | (63.2%) | (76.5%) | (84.6%) | (90.0%) | (66.7%) | (85.7%) | (40.0%) |

Data are presented as the number (and percentage) of patients that achieved ≥10% spot urine protein:creatinine ratios (uPCR) reduction from baseline out of total observations.

### Example 9. Effects of mAb 2419-1406 on Kidney Function

This Example describes that administration of mAb 2149-1406 to IgA neuropathy patients resulted in an improvement of kidney function and induction of kidney recovery.

In this example, IgA nephropathy patients were randomized 1:1:1:1 to receive either placebo: 2 mg/kg mAb 2419-1406:4 mg/kg mAb 2419-1406: or 8 mg/kg mAb 2419-1406 once per month, and the estimated glomerular filtration rate (eGFR) was measured once a month over 8 months. The median, mean, standard error of eGFR expressed as ml/min per body surface area of 1.73 m² were determined. Later time point data were masked to avoid the risk of unblinding.

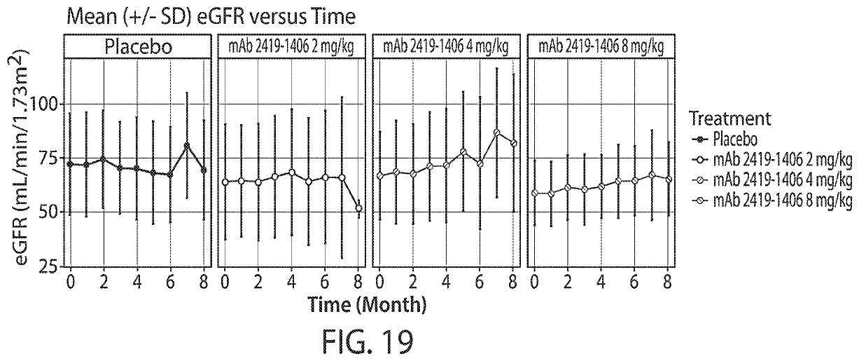

As shown in FIG. 19 and Table 30, patients on placebo demonstrated a gradual decrease in eGFR over time, indicative of progression of IgA neuropathy. Patients receiving 2 mg/kg mAb 2419-1406 monthly demonstrated stabilization of eGFR, whereas patients in the 4 mg/kg or 8 mg/kg mAb 2419-1406 dose groups demonstrated an increase in eGFR (i.e. positive slope vs. time) from baseline (t=0). Given the role of mAb 2419-1406 in removal of autoantigen (gd-IgA), autoantibodies, and immune complexes of IgA/IgM, this increase in eGFR slope upon mAb 2419-1406 administration is indicative of restoration of kidney function and induction of kidney recovery.

In summary, administration of mAb 2419-1406 to IgA neuropathy patients increased eGFR, improved kidney function, and induced kidney recovery.

TABLE 30
Estimated Glomerular Filtration Rate (eGFR) by mAb 2419-1406 Dose and Time

| | eGFR (mL/min/1.73 m²) Median (Mean ± SD) | | | | | | | | |
|---|---|---|---|---|---|---|---|---|---|
| Arm | Month 0 | Month 1 | Month 2 | Month 3 | Month 4 | Month 5 | Month 6 | Month 7 | Month 8 |
| Placebo | 70 (72 ± 23), | 68 (72 ± 24)., | 74 (74 ± 23), | 64 (70 ± 21), | 63 (70 ± 23), | 70 (68 ± 24), | 66 (67 ± 22), | 77 (80 ± 25), | 63 (69 ± 23), |
| | n = 27 | n = 26 | n = 22 | n = 18 | n = 15 | n = 14 | n = 8 | n = 6 | n = 4 |
| 2 mg/kg | 58 (64 ± 27), | 58 (64 ± 26), | 56 (64 ± 27), | 58 (66 ± 28), | 62 (68 ± 29), | 51 (64 ± 29), | 52 (66 ± 30), | 58 (66 ± 37), | 53 (51 ± 4), |
| | n = 25 | n = 22 | n = 19 | n = 18 | n = 16 | n = 12 | n = 11 | n = 5 | n = 3 |
| 4 mg/kg | 64 (67 ± 20), | 60 (68 ± 24), | 64 (68 ± 23), | 68 (71 ± 25), | 61 (71 ± 26), | 71 (78 ± 28), | 58 (72 ± 31), | 84 (87 ± 30), | 79 (82 ± 23), |
| | n = 25 | n = 24 | n = 22 | n = 17 | n = 15 | n = 11 | n = 12 | n = 8 | n = 6 |
| 8 mg/kg | 56 (59 ± 15), | 56 (58 ± 15), | 55 (61 ± 15), | 55 (60 ± 16), | 62 (62 ± 15), | 62 (64 ± 17), | 62 (64 ± 16), | 62 (67 ± 20), | 61 (65 ± 17), |
| | n = 25 | n = 23 | n = 21 | n = 18 | n = 14 | n = 12 | n = 10 | n = 8 | n = 5 |

Data are presented as median (mean ± standard deviation (SD)).
n = number of patients.

### Example 10. Study Assessing Efficacy and Safety of mAb 2419-1406 in IgA Nephrophathy Patients

This Example describes interim analyses of a Phase 2 study in which IgA nephropathy patients were administered mAb 2419-1406, a humanized IgG2 monoclonal antibody that binds APRIL.

Participants with IgA nephropathy were screened and enrolled in the study based on the following criteria: stable and maximally tolerated ACEI/ARBs for at least 3 months, eGFR>30 ml/min/1.73 m², and proteinuria>1.0 g/d or uPCR>0.75 g/g. Eligible participants (approximately 155 participants) were randomized to receive 12 monthly doses of placebo, 2.0 mg/kg, 4.0 mg/kg, or 8.0 mg/kg of mAb 2419-1406. The following data presented in this Example are from interim analyses in which 72 participants (approximately 50% of the total participants) completed at least 9 months of the 12-month treatment course.

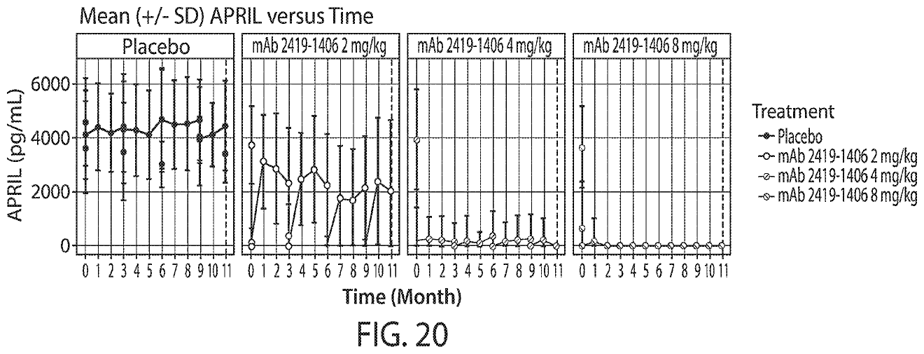

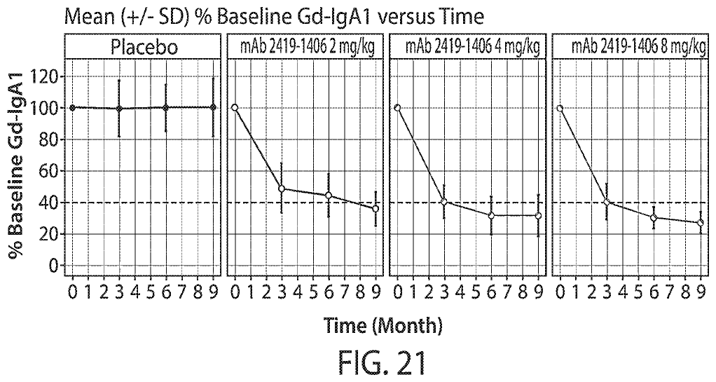

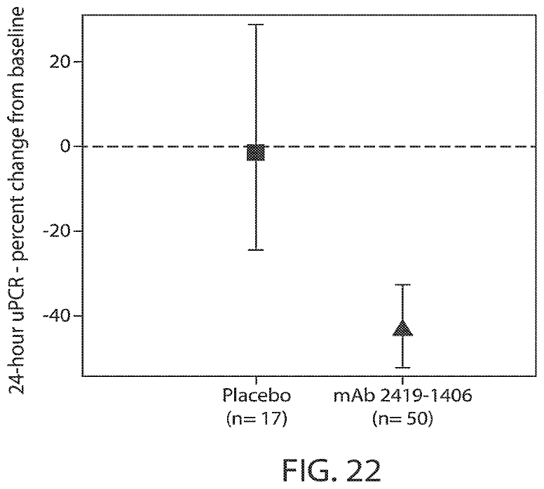

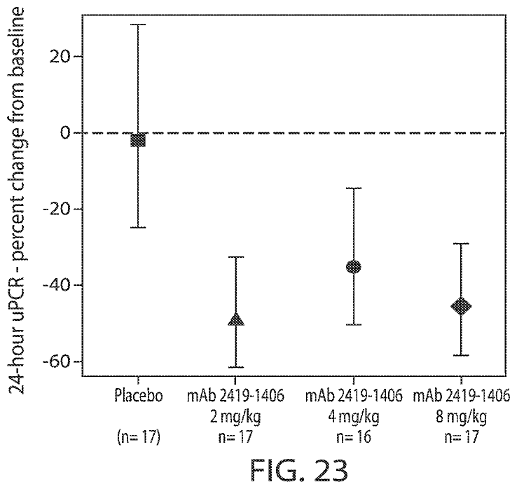

Administration of 4 mg/kg or 8 mg/kg of mAb 2419-1406 induced immediate and near-complete suppression of APRIL levels, while APRIL levels remained near baseline in placebo-treated participants. In the 2 mg/kg dose arm, APRIL levels were lower than those in the placebo arm, with suppression following dosing and an increase prior to the next dose (FIG. 20). Group mean levels of galactose-deficient IgA1 (Gd-IgA1) were suppressed by at least 60% in mAb 2419-1406 recipients (FIG. 21). Therefore, both APRIL and Gd-IgA1 decreased in a dose-dependent manner. Among pooled mAb 2419-1406 recipients, there was a 43% placebo-adjusted reduction from baseline in 24-hour uPCR values at Month 9 (FIG. 22). When each mAb 2419-1406 dose arm was examined separately, 24-hour uPCR levels were reduced by 34-48% as compared to baseline at Month 9 (FIG. 23).

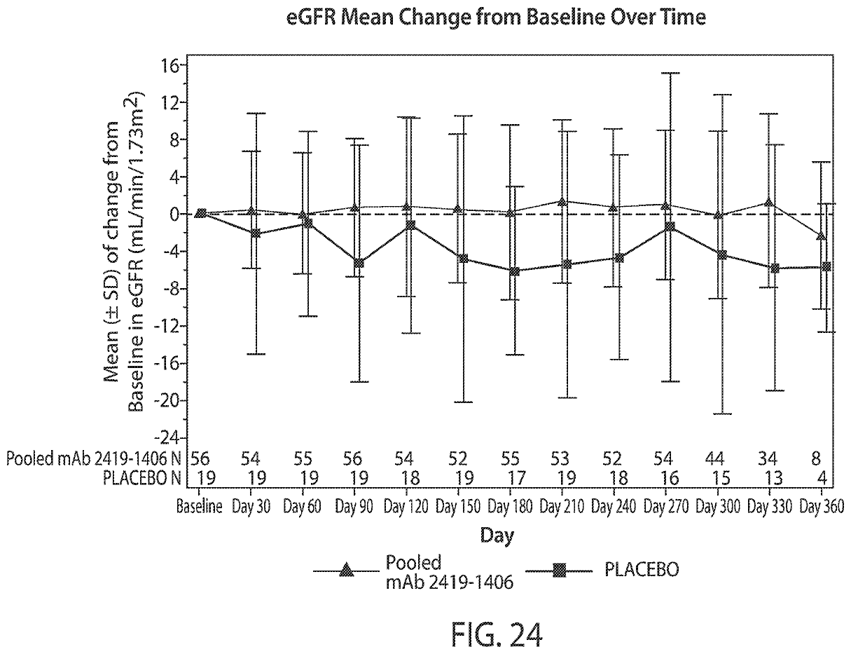

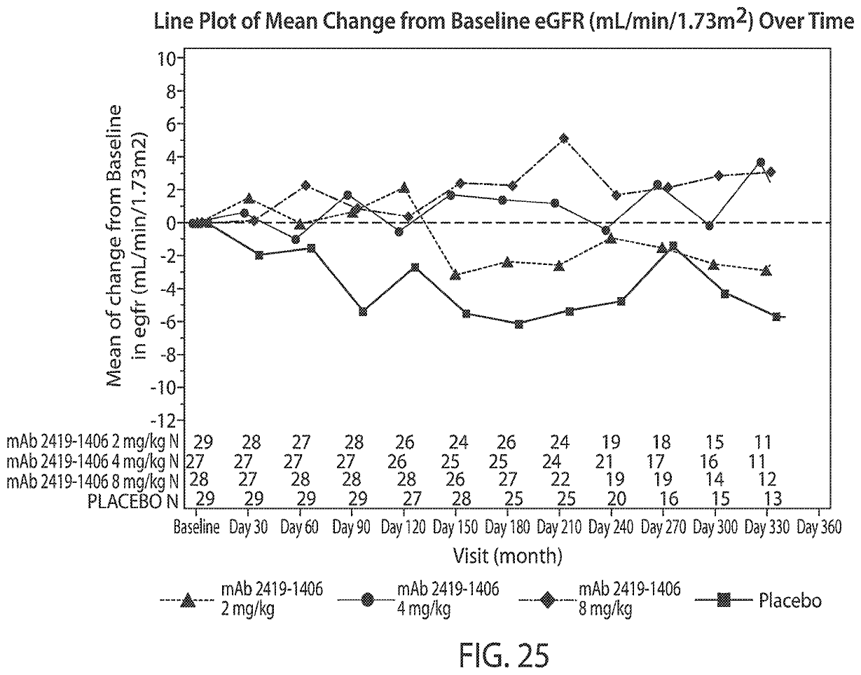

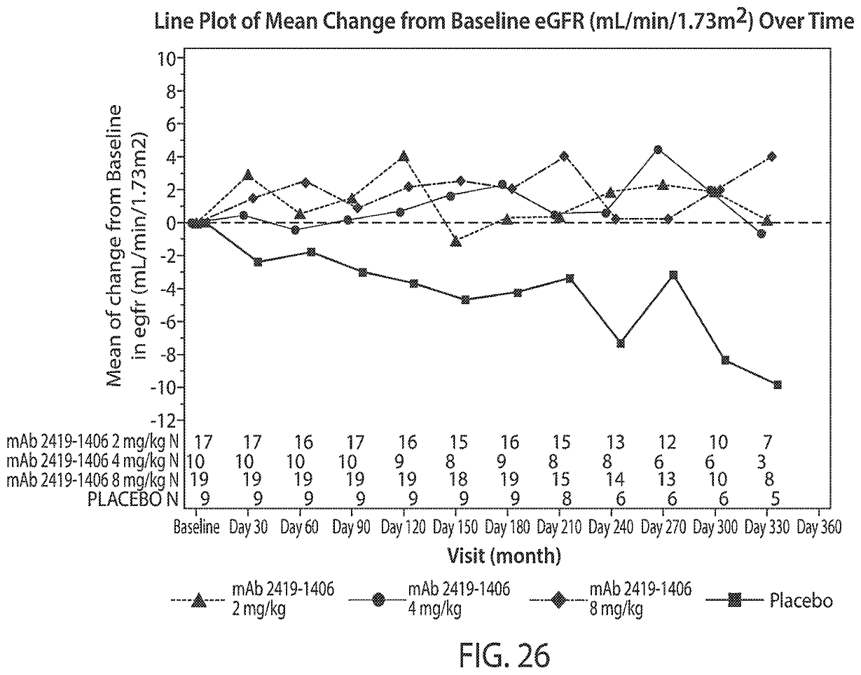

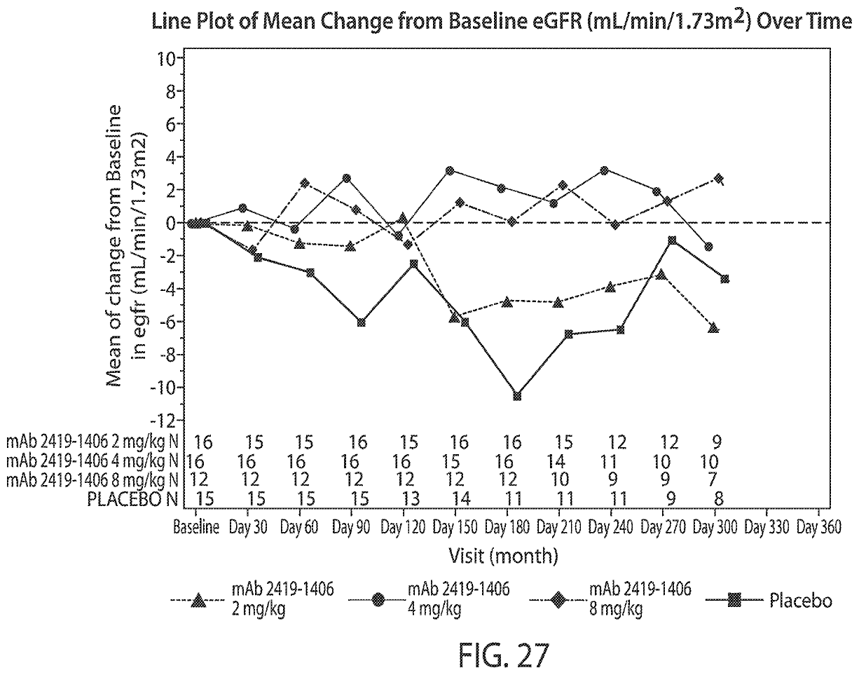

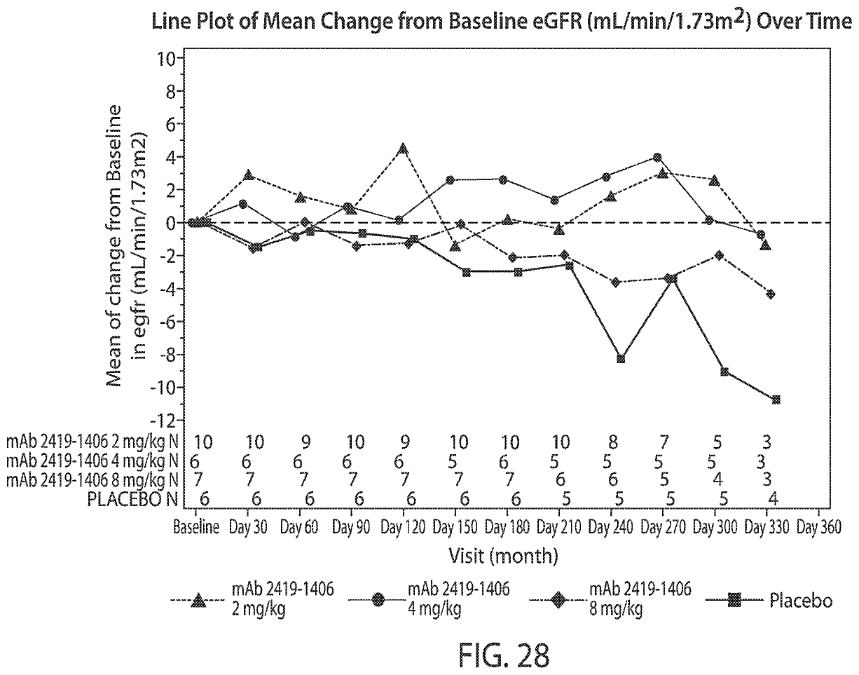

Estimated glomerular filtrate rates (eGFR) and slopes were measured in participants receiving placebo or 2.0 mg/kg, 4.0 mg/kg, or 8.0 mg/kg of mAb 2419-1406. Among participants with >9 months on study, the estimated annualized eGFR slope was stable (+1.2 mL/min/1.73 m²) in pooled mAb 2419-1406 recipients versus declining (−6.5 mL/min/1.73 m²) in the placebo group, with a slope difference of +7.7 mL/min/1.73 m² (FIG. 24). When the dose arms were examined separately in participants with >9 months on study, eGFR decreased relative to baseline in placebo-treated participants over the course of the study, while eGFR was preserved in the mAb 2419-1406 dose arms, with increased eGFR observed in participants receiving 4 mg/kg or 8 mg/kg mAb 2419-1406 (FIG. 25; Table 31). The 95% confidence interval (CI) around the slope difference excludes 0 for the 4 mg/kg and 8 mg/kg dose arms. In subgroup analyses of patients with (1) baseline eGFR≤60 mL/min/1.73 m², (2) baseline proteinuria>2.0 g/day (in which 24-hour urine protein>2.0 g/day or 24-hour uPCR>1.5 g/g). or (3) baseline proteinuria>2.0 g/day and baseline eGFR≤60 mL/min/1.73 m², eGFR declined in placebo-treated participants, while eGFR was stabilized or improved in the mAb 2419-1406 dose arms (FIGS. 26-28, Tables 32-34).

TABLE 31
Analysis of linear mixed model with random effects
for annualized eGFR slope estimated over 12 Months

| | | Difference vs | |
|---|---|---|---|
| Arm | Slope mL/min/1.73 m² | Placebo | 95% CI (difference) |
| Placebo | −7.4 | | |
| 2 mg/kg | −5.0 | +2.4 | −4.1 to +8.9 |
| 4 mg/kg | +1.7 | +9.2 | +3.1 to +15.2 |
| 8 mg/kg | +3.7 | +11.1 | +5.1 to +17.1 |

TABLE 34
Analysis of Linear Mixed Model with Random Effects
for Annualized eGFR Slope Estimated over 12 Months
- Subgroup with baseline proteinuria > 2.0 g/day*
and baseline eGFR ≤ 60 mL/min/1.73 m²

| | | Difference vs | |
|---|---|---|---|
| Arm | Slope mL/min/1.73 m² | Placebo | 95% CI (difference) |
| Placebo | −9.9 | | |
| 2 mg/kg | −3.1 | +6.8 | −3.6 to +17.2 |
| 4 mg/kg | +3.2 | +13.1 | 1.5 to +24.8 |
| 8 mg/kg | −4.3 | +5.6 | −4.4 to +15.7 |

*24-hour urine protein > 2.0 g/day or 24-hour uPCR > 1.5 g/g

TABLE 32
Analysis of Linear Mixed Model with Random Effects
for Annualized eGFR Slope Estimated over 12 Months
- Subgroup with baseline eGFR ≥ 60 mL/min/1.73 m²

| | | Difference vs | |
|---|---|---|---|
| Arm | Slope mL/min/1.73 m² | Placebo | 95% CI (difference) |
| Placebo | −7.8 | | |
| 2 mg/kg | −3.0 | +4.8 | −3.0 to +12.6 |
| 4 mg/kg | +1.4 | +9.2 | −0.5 to +18.9 |
| 8 mg/kg | +2.8 | +10.6 | +2.5 to +18.6 |

TABLE 33
Analysis of Linear Mixed Model with Random Effects
for Annualized eGFR Slope Estimated over 12 Months
- Subgroup with baseline proteinuria >2.0 g/day*

| | | Difference vs | |
|---|---|---|---|
| Arm | Slope mL/min/1.73 m² | Placebo | 95% CI (difference) |
| Placebo | −8.9 | | |
| 2 mg/kg | −7.4 | +1.5 | −7.4 to +10.3 |
| 4 mg/kg | +2.3 | +11.2 | +2.8 to +19.6 |
| 8 mg/kg | −0.7 | +8.2 | −0.3 to +16.6 |

*24-hour urine protein > 2.0 g/day or 24-hour uPCR > 1.5 g/g

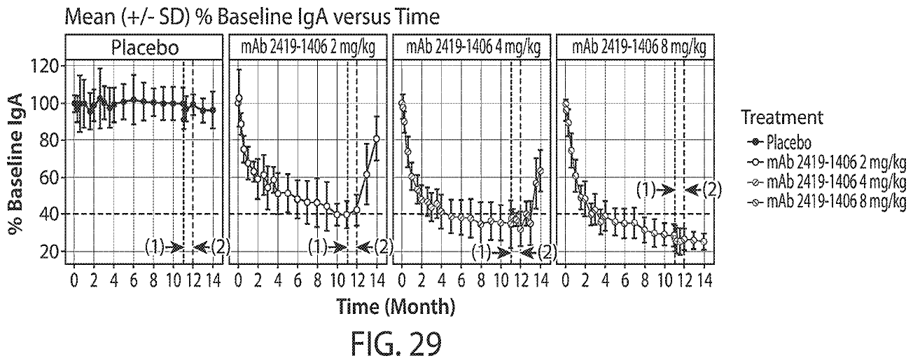

Total IgA levels were assessed following administration of placebo or mAb 2419-1406. While minimal changes in total IgA levels were observed in placebo-treated patients, the levels of total IgA decreased in a dose-dependent manner following mAb 2419-1406 dosing (FIG. 29).

When treatment-emergent adverse events (TEAEs) were assessed, no serious adverse events were considered study-drug related, and most adverse effects were limited to mild or moderate severity.

In summary, this Phase 2 interim analysis of mAb 2419-1406 demonstrated acceptable safety and tolerability with robust uPCR reduction associated with eGFR stability, versus persistent proteinuria and progressive eGFR decline in placebo recipients. Administration of mAb 2419-1406 to IgA nephropathy patients resulted in rapid and complete suppression of APRIL and >60% reduction in mean total IgA and Gd-IgA1 at all dose levels (2 mg/kg, 4 mg/kg, and 8 mg/kg of mAb 2419-1406). Robust and clinically meaningful reduction in proteinuria was observed, with 43% reduction in uPCR in pooled recipients of mAb 2419-1406 vs. placebo recipients. Increased eGFR trend was observed in patients treated with mAb 2419-1406 at the 4 mg/kg and 8 mg/kg dose levels, suggesting a reduction in glomerular inflammation. Such results were consistently observed across high-risk patient subgroups, including patients at very high risk of progression (e.g., baseline proteinuria>2.0 g/day and baseline eGFR≤60 mL/min/1.73 m²).

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of improving kidney function, the method comprising administering to a human subject in need thereof an anti-APRIL antibody molecule,
wherein the antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3),
wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or
wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; and
wherein the method reverses or prevents progression of decreased kidney function in the subject,
thereby improving kidney function.

2-5. (canceled)

6. The method of claim 1, wherein the method improves kidney function in the subject
(i) within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after administration of the antibody molecule;
(ii) after 1,2,3,4,5,6,7,8,9, 10, 11, 12, 13, 14, 15, 16, 17, 18, 19, 20, 21, 22, 23, 24, 25, 26, 27, 28, 29, 30, 31, 32, 33, 34, 35, or 36 administrations of the antibody molecule; and/or
(iii) for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months.

7-8. (canceled)

9. The method of claim 1, wherein the method preserves estimated glomerular filtrate rate (eGFR) in the kidney.

10. The method of claim 1, wherein the method reduces proteinuria in the kidney.

11-20. (canceled)

21. The method of claim 1, wherein the method induces kidney recovery.

22. The method of claim 1, wherein the method induces kidney regeneration.

23. The method of claim 1, wherein the method reduces an autoantibody response.

24. The method of claim 1, wherein the antibody molecule comprises:
(a) a VH comprising the amino acid sequence of SEQ ID NO: 296, an amino acid sequence having at least 85%, 90%, 95%, 96%, 97%, 98%, or 99% identity thereto, or an amino acid sequence differing by no more than 1, 2, 3, 4, 5, 6, 7, 8, 9, or 10 amino acids therefrom; and/or
(b) a VL comprising the amino acid sequence of SEQ ID NO: 286, or an amino acid sequence having at least 85%, 90%, 95%, 96%, 97%, 98%, or 99% identity thereto, or an amino acid sequence differing by no more than 1, 2, 3, 4, 5, 6, 7, 8, 9, or 10 amino acids therefrom.

25-26. (canceled)

27. The method of claim 1, wherein the antibody molecule comprises a heavy chain constant region of IgG2 and a light chain constant region of kappa.

28. The method of claim 1, wherein the antibody molecule is sibeprenlimab.

29. The method of claim 1 wherein the antibody molecule is administered:
(i) no more than once a month, once a month, once every two months, once every three months, once every four weeks, or once every eight weeks;
(ii) at a dose of 2 mg/kg, 4 mg/kg, 8 mg/kg, 200 mg, 400 mg, 600 mg, or 800 mg;
(iii) at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 times; and/or
(iv) intravenously or subcutaneously.

30-34. (canceled)

35. The method of claim 1, wherein the antibody molecule is administered at a dose of 4 mg/kg or 8 mg/kg, once a month intravenously, or 400 mg once every four weeks subcutaneously, and the administration results in an increase in eGFR within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after the administration, wherein eGFR is increased by at least 10%, 20%, 30%, 40%, 50%, 60%, 70%, 75%, 80%, 85%, 90%, 95%, 96%, 97%, 98%, or 99%.

36-37. (canceled)

38. The method of claim 1, wherein administration of the antibody molecule results in a decrease in the level of serum IgA, serum a-g-IgA, serum IgG, serum IgM, and/or uPCR compared to the level of serum IgA, serum a-g-IPA, serum IgG, serum IgM, and/or uPCR prior to the administration.

39. The method of claim 38, wherein the decrease in the level of serum IgA serum a-g-IgA, serum IgG, serum IgM, and/or uPCR is maintained for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months; and/or wherein the level of serum IgA, serum a-g-IgA, serum IgG, serum IgM, and/or uPCR is decreased by at least 40%, 50%, 60%, 70%, 75%, 80%, 85%, 90%, 95%, 96%, 97%, 98%, or 99%, within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after the administration.

40-56. (canceled)

57. The method of claim 1, wherein the subject has a disorder, or is at risk of having a disorder, wherein the disorder is a chronic kidney disease (CKD), an autoantibody-related disorder, an IgM-mediated disorder, a glomerulonephritis, IgG nephropathy (IgAN), lupus nephritis, Henoch-Schönlein purpura (HSP), vasculitis, lupus, atypical hemolytic uremic syndrome (aHUS), membranoproliferative glomerulonephritis (MPGN), primary membranous nephropathy, Goodpasture's disease, cold agglutinin disease, anti-MAG neuropathy, anti-GM1 neuropathy, Sjogren's syndrome, post transplant IgA nephropathy, post transplant recurrence of glomerular disease, or post transplant IgA vasculitis.

58-59. (canceled)

60. The method of claim 57, wherein the subject
(i) is at risk of kidney failure;
(ii) is a pediatric patient;
(iii) is not immunocompromised; and/or
(iv) has proteinuria greater than or equal to a uPCR of 0.75 g/g or urine protein 1 g/day prior to the administration.

61-77. (canceled)

78. The method of claim 1, further comprising administering a second therapeutic agent or modality to the subject.

79. The method of claim 78, wherein the second therapeutic agent comprises a TACI-IgG fusion protein, a C1 inhibitor, or an anti-CD20 antibody.

80-81. (canceled)

82. A method of treating IgA nephropathy, the method comprising:
responsive to an identification of a human subject who will benefit from administration of an anti-APRIL antibody molecule, administering to the subject an effective amount of the anti-APRIL antibody molecule,
wherein the anti-APRIL antibody molecule is administered no more than once a month,
wherein the benefit comprises one two, three, or all of the following:
(i) reduction of the level of APRIL by 90% or more, within one-month administration of the anti-APRIL antibody molecule, compared to the subject's baseline level of APRIL;
(ii) reduction of the level of galactose-deficient IgA1 (Gd-IgA1) by 60% or more, within nine-month administration of the anti-APRIL antibody molecule, compared to the subject's baseline level of Gd-IgA;
(iii) reduction of 24-hour urine protein creatinine ratio (uPCR) by 30% or more, within nine-month administration of the anti-APRIL antibody molecule, compared to the subject's baseline uPCR; or
(iv) preserving average eGFR over a period of at least 12 months after administration of the anti-APRIL antibody molecule, compared to the subject's baseline eGFR;
wherein the anti-APRIL antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3), and
wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or
wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16.

83-96. (canceled)

97. A method of treating IgA nephropathy, the method comprising administering to a human subject in need thereof an effective amount of an anti-APRIL antibody molecule,
wherein the subject has, or is identified to have, (a) a proteinuria level greater than 2.0 g/day, (b) an estimated glomerular filtration rate (eGFR) less than 60 mL/min/1.73 m², or (c) both (a) and (b),
wherein the antibody molecule comprises a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3), and
wherein the VH comprises an HCDR1 comprising the amino acid sequence of SEQ ID NO: 11; an HCDR2 comprising the amino acid sequence of SEQ ID NO: 12, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16; or
wherein the VH comprises an HCDR1 comprising the amino acid sequence of SEQ ID NO: 17; an HCDR2 comprising the amino acid sequence of SEQ ID NO: 282, and an HCDR3 comprising the amino acid sequence of SEQ ID NO: 13; and the VL comprises an LCDR1 comprising the amino acid sequence of SEQ ID NO: 280; an LCDR2 comprising the amino acid sequence of SEQ ID NO: 285, and an LCDR3 comprising the amino acid sequence of SEQ ID NO: 16,
wherein the anti-APRIL antibody molecule is administered no more than once a month,
thereby treating IgA nephropathy.

98-165. (canceled)
